(19) United States
(12) Patent Application Publication
(10) Pub. No.: US 2021/0253652 A1
(43) Pub. Date: Aug. 19, 2021

(54) EXPRESSION OF HUMAN FOXP3 IN GENE EDITED T CELLS

(71) Applicant: Seattle Children's Hospital (dba Seattle Children's Research Institute), Seattle, WA (US)

(72) Inventors: Andrew M. Scharenberg, Seattle, WA (US); David J. Rawlings, Seattle, WA (US); Karen Sommer, Seattle, WA (US); Yuchi Chiang Honaker, Seattle, WA (US); Iram F. Khan, Seattle, WA (US); Troy Torgerson, Seattle, WA (US)

(21) Appl. No.: 16/981,213

(22) PCT Filed: Apr. 25, 2019
(86) PCT No.: PCT/US2019/029159

§ 371 Date: Sep. 15, 2020

Related U.S. Application Data
(60) Provisional application No. 62/663,561, filed on Apr. 27, 2018.
(60) Provisional application No. 62/773,414, filed on Nov. 30, 2018.

(51) Int. Cl. C07K 14/47; C12N 15/86; C12N 15/90; A61K 35/17; C12N 5/0783; A61P 37/06

(52) U.S. Cl. CPC C07K 14/4702; C12N 15/86; C12N 15/907; A61K 35/17; C12N 5/0636; C12N 2501/60; C12N 2800/80; C12N 2310/20; C12N 2750/14143; C12N 2510/00; A61P 37/06

(57) ABSTRACT

Aspects of the invention described herein concern targeting of a FOXP3 cDNA, e.g., full-length human-codon optimized, into a FOXP3 locus or a non-FOXP3 locus so as to provide constitutive or regulated FOXP3 expression in a primary human lymphocyte. The compositions and materials described herein provide specificity for CRISPR/Cas-mediated gene regulation of murine, non-human primates or human FOXP3. Guide RNA sequences are used to target the FOXP3, AAVS1, and other candidate loci for CRISPR/Cas-mediated gene regulation, and gene delivery cassettes for HDR based gene-modification are provided. The alternative compositions described herein can be delivered in the form of Ribonucleoprotein (RNP) and may be used to target human and/or non-human primate FOXP3. Reagents are comprised of novel guide RNA sequences and can generate high frequency of on-target cleavage in combination with a Cas protein and novel gene delivery cassettes including FOXP3 cDNA+/−other cis linked gene products.

### CROSS-REFERENCE TO RELATED APPLICATIONS

This application claims priority to U.S. Prov. App. No. 62/663,561 filed Apr. 27, 2018 entitled “EXPRESSION OF MRNA ENCODING HUMAN FOXP3 FROM A NON-FOXP3 OR A FOXP3 GENETIC LOCI IN GENE EDITED T CELLS”, and U.S. Prov. App. No. 62/773,414 filed Nov. 30, 2018 entitled “EXPRESSION OF HUMAN FOXP3 IN GENE EDITED T CELLS”, which are each incorporated by reference in its entireties for all purposes.

### REFERENCE TO SEQUENCE LISTING

The present application is being filed along with a Sequence Listing in electronic format. The Sequence Listing is provided as a file entitled SCRI187WOSEQLIST, created Apr. 25, 2019, which is approximately 496 Kb in size. The information in the electronic format of the Sequence Listing is incorporated herein by reference in its entirety.

### FIELD

Aspects of the invention described herein concern the incorporation of a FOXP3 coding sequence into a FOXP3 locus or a non-FOXP3 locus in lymphocytic cells to provide constitutive or regulated FOXP3 expression in the edited lymphocytic cells, such as T cells.

### BACKGROUND

Lentiviral gene transfer of FOXP3 (also known as forkhead box protein P3, forkhead box P3, AAID, DIETER, IPEX, JM2, PIDX, XPID, or scurfin) has been previously described by Chen, C. et al. (2011). Transplant. Proc. 43(5):2031-2048, Passerini, L. et al. (2013). Sci. Transl. Med., 5(215):215ra174, and Passerini, L. et al. (2017). Front. Immunol. 8:1282; each of which is hereby expressly incorporated by reference in its entirety. Passerini et al. (2017) had previously reported the development of methods to restore T_{reg} function in T lymphocytes from patients carrying mutations in FOXP3. As described by Passerini et al. (2017), lentiviral mediated gene transfer was used in CD4+ T cells and effector T cells which were converted into regulatory T cells, which exhibited characteristics of T_{reg}-like cells and endowed the cells with potent in vitro and in vivo suppressive activity. Passerini et al. (2013) also demonstrated conversion of CD4+ T cells into T_{reg} cells after lentiviral mediated FOXP3 gene transfer, in which the cells were shown to be stable in inflammatory conditions. Chen et al. (2011) also describes the adoptive transfer of engineered T cells, in which the T cells were infected with a lentiviral vector encoding a FOXP3-IRES-GFP fragment. These cells were shown to protect recipients from GVHD in a murine model. The need for new approaches to express and regulate FOXP3 in a primary human lymphocytes is manifest.

Many investigators are interested in treating auto-immune diseases with regulatory T cells, due to the possibility for these cells to induce antigen specific tolerance. There are many forms of regulatory T cells (“T_{regs}”), with current nomenclature dividing T_{regs} into those which are generated in the thymus in the course of T cell development, denoted as thymic regulatory T cells or “tT_{regs}”, and peripherally induced regulatory T cells, denoted as peripheral regulatory T cells or “pT_{regs}.”

A key aspect of regulatory T cell biology is the expression of the transcription factor FOXP3 (also known as forkhead box protein P3, forkhead box P3, AAID, DIETER, IPEX, JM2, PIDX, XPID, and scurfin). FOXP3 is thought to be required to specify the regulatory T cell lineage. This concept is based on the observation that humans who lack FOXP3 develop severe autoimmune disease starting in the neonatal period. The use of either tT_{regs} or pT_{regs} for therapy of autoimmune disease may not be optimal because FOXP3 expression is believed to be subject to epigenetic regulation. In tT_{regs}, an upstream region in the FOXP3 gene known as the “thymus specific demethylated region” is completely demethylated, a state which is thought to result in stable FOXP3 expression. Generally, full demethylation is not observed in pT_{regs}. Under inflammatory conditions, FOXP3 may be silenced epigenetically in pT_{regs}, and possibly tT_{regs}, potentially resulting in conversion of pT_{regs} to pro-inflammatory CD4+ T cells. The lack of stability of pT_{regs} is a significant concern, as the use of infusion of pT_{regs} that revert to an inflammatory phenotype could exacerbate auto-immune symptoms.

However, many approaches utilizing lentiviral constructs result in random integration into a cell's genome, which could potentially disrupt a tumor suppressor gene or activate a proto-oncogene. In addition, the integration site could be in a genomic region characterized by poor expression, and thus fail to result in stable expression of FOXP3.

### SUMMARY

An aspect of the invention is a system comprising: a deoxyribonucleic acid (DNA) endonuclease or nucleic acid encoding the DNA endonuclease; a guide RNA (gRNA) comprising a spacer sequence that is complementary to a sequence within a FOXP3 locus, AAVS1 locus, or a TCRa (TRAC) locus in a lymphocytic cell (e.g., a T cell), or a nucleic acid encoding the gRNA; and a donor template comprising a nucleic acid sequence encoding a FOXP3 protein or a functional derivative thereof. In some embodiments, the gRNA comprises: i) a spacer sequence from any one of SEQ ID NOs: 1-7, 15-20, 27-29, 33, and 34, or a variant thereof having no more than 3 mismatches compared to any one of SEQ ID NOs: 1-7, 15-20, 27-29, 33, and 34; ii) a spacer sequence from any one of SEQ ID NOs: 1-7 or a variant thereof having no more than 3 mismatches compared to any one of SEQ ID NOs: 1-7; or iii) a spacer sequence from any one of SEQ ID NOs: 2, 3, and 5 or a variant thereof having no more than 3 mismatches compared to any one of SEQ ID NOs: 2, 3, and 5. In some embodiments, the FOXP3 or functional derivative thereof is a wild-type human FOXP3. In some embodiments, the DNA endonuclease is a Cas endonuclease. In some embodiments, the DNA endonuclease is a Cas9. In some embodiments, the nucleic acid encoding the DNA endonuclease is an mRNA. In some embodiments, the donor template is encoded in an adeno-associated virus (AAV) vector. In some embodiments, the DNA endonuclease or nucleic acid encoding the DNA endonuclease is formulated in a liposome or lipid nanoparticle.

Also described herein is a method of editing a genome in a lymphocytic cell, the method comprising providing any one of the systems described herein to the cell. In some embodiments, the cell is not a germ cell.

The present disclosure also describes a genetically modified lymphocytic cell, and a composition comprising a genetically modified lymphocytic cell, in which the genome of the cell is edited by any one of the methods described herein.

Further described is a method of treating a disease or condition associated with FOXP3 in a subject, comprising providing any one of the systems described herein to a lymphocytic cell in the subject. The disease or condition can be an inflammatory disease or an autoimmune disease, such as IPEX syndrome or Graft-versus-Host disease (GVHD). Some embodiments include a medicament for use in treating a disease or condition associated with FOXP3 in a subject. More embodiments concern a genetically modified lymphocytic cell in which the genome of the cell is edited by one of the methods described herein for use in inhibiting or treating a disease or condition associated with FOXP3, such as an inflammatory disease or an autoimmune disease such as IPEX syndrome or Graft-versus-Host disease (GVHD). Additional embodiments concern use of a genetically modified lymphocytic cell in which the genome of the cell is edited by any one of the methods herein as a medicament.

### BRIEF DESCRIPTION OF THE DRAWINGS

FIG. 1 shows the design of AAV5 donor templates with varying promoter elements with GFP coding sequence in frame.

FIG. 2 shows the design of AAV5 donor templates with an MND, sEF1a, or PGK promoter element, with LNFGR and P2A coding sequences in frame.

FIG. 3 shows a bar graph depicting FOXP3 MFI in each experiment.

FIG. 4 shows results of the gene editing of T cells derived from a non-human Primate: RhesusCD4+electroporation.

FIG. 5 shows results of the gene editing of T cells derived from a non-human Primate: Rhesus CD4+AAV Serotyping. Two different guide RNAs and their variants were designed to target the last exon of a human TRAC gene. The guide RNAs were tested in the absence or presence of 3 different gene-trap(GT) AAV donor templates described in FIG. 6 to determine the editing (NHEJ and HDR) efficiency.

FIG. 6 shows exemplary TCRa gene trap constructs.

FIG. 7 shows compilation of intracellular flow cytometry results to determine expression levels of inflammatory cytokines IL-2, IFNγ and TNFα. P values were determined using Student's unpaired T test.

FIG. 8 shows a Kaplan-Meier curve showing the percent survival of each cohort over time in days. The number of animals in each cohort is indicated in the legend, and represents data from two experiments using two different healthy T cell donors. P values for the mock-edited and edT_{reg} cohorts are relative to the T_{eff} only group.

FIG. 9 is a schematic of AAV donor templates #1303, FWD 07UCOE, RVS 07UCOE, and no 07UCOE control.

FIG. 10 show GVHD scores of mice treated with different edT_{reg} preparations in the in vivo mouse xenoGVHD experiment of Example 19.

FIG. 11 shows immunophenotyping analysis of animals in the mouse xenoGVHD study of Example 19, showing the percentage of cells in either LNGFR− or LNGFR+ cell populations.

FIG. 12 shows data for the in vivo xenoGVHD experiment of Example 19. Percent survival of mouse cohorts treated with T_{eff} only, T_{eff}+mock edited T cells, and T_{eff}+edT_{reg}, administered intraperitoneally (IP) or intravenously (IV), are shown.

FIG. 13 shows the results of an experiment to edit CD4+ T cells derived from an IPEX subject according to Example 20, using Cas9/gRNA-T9 (1:2.5 ratio) RNP and AAV donor template #3066. Bar graphs depicting % HDR efficacy and cytokine profile are shown.

FIG. 14 shows the results of an experiment to edit the CD4+ T cells derived from an IPEX subject according to Example 20, using Cas9/gRNA-T9 (1:2.5 ratio) RNP and AAV donor template #3080 as shown in the figure. Bar graphs depicting % HDR efficacy and cytokine profile are shown.

FIGS. 15-17 show in vitro and in vivo results of edT_{reg}-mediated suppression assays from three different batches of edT_{regs}. FIG. 15 depicts the in vitro suppression under the Method 1 assay protocol of mock edited CD4+ cells, CD4+ cells edited according to Example 10 with AAV donor template #3066 (“3066”), and CD4+ cells edited according to Example 10 with AAV donor template #3080 (“3080”) (left and middle graph). Irradiation and T_{reg}:T_{eff} ratios were used as indicated on the x-axis. Also depicted are results from an in vivo experiment in the murine CATI model described in Example 13 using the same batch of edT_{regs} (right graph). FIG. 16 depicts in vitro suppression under the Method 2 assay protocol of mock edited CD4+ cells, and Batch #2 of CD4+ cells edited according to Example 10 with AAV donor template #3066 (left and middle graph). T_{reg}:T_{eff} ratios were used as indicated on the x-axis. Also depicted are results from an in vivo experiment in the murine CATI model described in Example 13 using Batch #2 of edT_{regs} (right graph). FIG. 17 depicts in vitro suppression under the Method 2 assay protocol of mock edited CD4+ cells and Batch #3 of CD4+ cells edited according to Example 10 with AAV donor template #3066 (left graph). T_{reg}:T_{eff} ratios were used as indicated on the x-axis. Also depicted are results from an in vivo experiment in the murine CATI model described in Example 13 using Batch #3 of edT_{regs} (right graph).

### DETAILED DESCRIPTION

Expression of FOXP3 from a DNA sequence (e.g., a codon-optimized DNA sequence, such as for expression in human cells) that is integrated in a FOXP3 locus or a non-FOXP3 locus is described herein. Guide RNAs are used to target a FOXP3 locus (e.g., murine, human, and nonhuman primate) or a non-FOXP3 locus for CRISPR/Cas-mediated genome editing. Accordingly, aspects of the invention concern the utilization of novel guide RNAs in combination with Cas proteins to create DNA breaks at FOXP3 or non-FOXP3 loci to facilitate integration of a FOXP3 coding sequence. In some embodiments, the integration is by non-homologous end joining (NHEJ) or homology directed repair (HDR) in association with a donor template containing the FOXP3 coding sequence. Embodiments described herein can be used in combination with a broad range of selection markers such as LNGFR, RQR8, CISC/DISC/μDISC, or others, and can be multiplexed with editing of other loci or co-expression of other gene products, including cytokines.

As described in greater detail below, Applicant has identified guide RNAs which, in combination with a Cas protein and novel AAV donor templates containing gene delivery cassettes, generate a high frequency of on-target cleavage and integration of the gene delivery cassette into a FOXP3 locus in T cells, e.g., human T cells, to generate genome edited T cells that have the phenotype of T_{reg} cells, also referred to herein as “edT_{reg} cells”, “edT_{reg}”, or “edT_{regs}.” This approach to generate edT_{reg} cells was successfully used to effect an immunosuppressive phenotype in CD4+ T cells derived from a subject suffering from IPEX syndrome. In addition, sustained engraftment of the edT_{reg} cells in NSG recipient mice was achieved, resulting in a higher survival rate in the treated animals. These findings demonstrate that the genome editing systems such as the CRISPR/Cas systems described herein are capable efficient editing to effect expression of a human wild-type FOXP3 in human hematopoietic stem cells and sustained engraftment at levels that are predicted to provide a clinical benefit in diseases or disorders having aberrant FOXP3 function, e.g., following autologous adoptive cell therapy in IPEX subjects.

The use of CRISPR/Cas systems including gRNAs and donor templates configured to insert the FOXP3 coding sequences at an endogenous FOXP3 locus or non-FOXP3 locus offers a promising therapy for IPEX syndrome. Since IPEX syndrome can be caused by a diversity of mutations spread over the entire gene, inserting the entire FOXP3 cDNA (e.g., human codon optimized) at the start codon may be desired. Utilizing the endogenous FOXP3 promoter is expected to provide the necessary transcriptional signals required for acceptable levels of FOXP3 expression in the edited lymphocytes.

Previous techniques for expressing FOXP3 relied on expression via the endogenous FOXP3 gene or lentiviral gene transfer of FOXP3. Specifically, FOXP3 expression has been achieved by using lentiviral vector delivery or expression from the endogenous FOXP3 locus following gene editing. Existing lentiviral delivery methods for FOXP3 expression are problematic as expression is dependent upon random viral integration, leading to challenges with limited ability to regulate expression levels and viral silencing resulting in loss of expression. As disclosed in some of the embodiments described herein, site-specific gene-editing techniques, e.g., using TALEN or CRISPR/Cas systems, generated DNA breaks at an endogenous FOXP3 locus in lymphocytes. Thus, the gene-editing methods provided in the embodiments described herein provide for site-specific targeting and integration of FOXP3 coding sequences, which is believed to be a safer and more controlled approach.

As compared to TALEN- or Cas mRNA-based approaches, systems using ribonucleoprotein (RNP) complexes comprising a Cas polypeptide associated with a guide RNA (gRNA) are capable of higher targeted integration efficiencies, as RNPs may be immediately functional once delivered into cells. In some of the embodiments described herein, components of a CRISPR/Cas system are delivered to cells in the form of RNPs and used to target a human and/or non-human primate FOXP3 locus or other genetic loci, including AAVS1 (adeno-associated virus integration site 1) and TCRa (TRAC).

The embodiments herein may be used to express full-length and functional FOXP3 in human T cells and lead to acquisition of a regulatory or a suppressive phenotype. These cell products may be useful for treatment in a broad range of conditions, including without limitation IPEX, autoimmunity, graft-vs.-host disease and solid organ transplant. Other applications that are contemplated include, for example, FOXP3 gene disruption and/or site-specific gene integration in a mouse, human or non-human primate FOXP3 locus or a non-FOXP3 locus, constitutive or regulated expression of a gene-of-interest through mono-allellic or bi-allelic gene integration at an AAVS1 site or another locus, use of any of the above approaches in patient therapy with IPEX, and use of any of the above approaches to generate T_{reg} cell populations from CD34 cells for treatment or amelioration of autoimmune conditions.

The embodiments described herein can also be used to generate human T cells that have FOXP3 expression so as to modify the phenotype of the T cell, e.g., by endowing the T cell with a regulatory or suppressive phenotype. One of the benefits of this approach is that FOXP3 can be linked to the expression of an endogenous gene. Another benefit is that FOXP3 expression can be linked to co-expression of gene products that permit enrichment of gene edited cells or that mediate expansion using CISC/DISC in vitro or in vivo. Further, changes achieved using biallelic gene-editing can be used to enrich or enhance the function of these cell products.

Transcription of FOXP3 mRNA from a human codon-optimized DNA sequence that is integrated in a FOXP locus or a non-FOXP3 genetic locus is described herein. Guide RNA sequences are used to target FOXP3 of murine, human and nonhuman primate FOXP3 gene for CRISPR/Cas-mediated gene regulation. Accordingly, aspects of the invention concern the utilization of novel guide RNA sequences in combination with a Cas protein to create DNA breaks at human and non-human primate FOXP3 loci, and human AAVS1 locus to facilitate nonhomologus end joining (NHEJ)-mediated gene disruption or homology-derived recombination(HDR)-mediated gene integration in the absence or presence of repair donor template respectively. Several embodiments described herein can be used in combination with a broad range of selection markers such as LNGFR, RQR8, CISC/DISC/uDISC or others, and can be multiplexed with editing of other loci or co-expression of other gene products, including cytokines.

As described in greater detail below, Ribonucleoprotein (RNP) can be used to deliver these reagents so as to target human and/or non-human primate FOXP3. In some embodiments, the reagents comprise unique guide RNA sequences, which generate high frequency of on-target cleavage in combination with a Cas protein and novel gene delivery cassettes including FOXP3 cDNA+/−other cis linked gene products.

Previously, a lentiviral gene transfer of FOXP3 has been described. Lentiviral constructs are randomly integrated into the genome, and could potentially disrupt a tumor suppressor gene or activate a proto-oncogene. In addition, the integration site could be silenced, and thus fail to stably express FOXP3. By contrast, gene editing provides site-specific targeting and integration. Thus, gene editing may be a safer and better controlled approach. Compared to TALEN or Cas mRNA, RNP has higher efficiency as it is immediately functional once delivered into cells.

Also contemplated are methods to design AAV constructs in which homology arms are shortened in order to be efficiently packaged into AAV. The editing efficiency may be slightly reduced, but edited cells can be enriched by a selection marker such as LNGFR, or other approaches to overcome the editing efficiency.

The cells generated are engineered regulatory T cells using a CRISPR system in combination with a repair donor DNA template for adoptive immunotherapy across a broad range of clinical conditions, including cancer, autoimmunity, and organ transplant, or for treatment of the genetic immune disorder, IPEX. Also described herein are methods of disrupting the endogenous FOXP3 gene expression using a CRISPR system.

Evidence is provided herein that an engineering approach that stabilizes FOXP3 expression in T cells may allow for the generation of expanded populations of potentially suppressive T cells that are no longer susceptible to epigenetic modification of their suppressive function. As a result, such cells may have improved properties for therapeutic application.

In the embodiments described herein, the cells for therapeutic application are engineered to have stable FOXP3 expression through the use of a gene editing nuclease to modify the regulatory elements of the FOXP3 locus to provide for stable FOXP3 expression. In the exemplary data provided, a promoter was placed upstream of the FOXP3 coding exons (examples of constitutive promoters include EF1 alpha promoter, the PGK promoter, and/or the MND promoter, among many others) to drive FOXP3 expression. However, a variety of approaches are envisioned to modify the regulatory elements to allow for stable FOXP3 expression. By several approaches used to modify the endogenous regulatory elements, the claimed therapeutic cell exhibited constitutive expression of the native FOXP3 gene, such that it was no longer susceptible to regulation that could result in FOXP3 gene silencing and reversion to a non-suppressive cell phenotype. Accordingly, in the methods described herein, the problem of loss of FOXP3 expression due to epigenetic influences on the native regulatory sequences and promoter has been solved.

In some embodiments, a method of enforcing FOXP3 expression in a bulk population of CD34 cells is contemplated. In subjects with auto-immune disease or who are rejecting an organ graft, the endogenous TCR repertoire in the inflammatory T cell population includes TCR's that have the correct binding specificity to recognize the inflamed tissue or the foreign tissue in the organ. These T cells are thought to mediate the auto-inflammatory reaction or organ rejection. By converting a portion of the bulk T cell population to a regulatory phenotype, the TCR specificities present in the pro-inflammatory population will be represented in the therapeutic cell population. This is an improvement over therapies based on thymic regulatory T cells, which are thought to have a distinct and non-overlapping TCR repertoire from inflammatory T cells. In addition, presumably in patients with auto-immune disease or organ rejection, the existing tT_{reg} population has failed to produce the tolerance necessary to avoid inflammation. The methods described herein can be used for therapy of auto-immune disease and for induction of tolerance to transplanted organs.

A significant disadvantage is the need to use gene editing tools that can efficiently carry out the recombination at the FOXP3 locus. As such, the methods provided show that the use of either TALEN or CAS/CRISPR nucleases can carry this reaction out efficiently, but in principle, any nuclease platform would serve equally well.

The regulatory T cell therapies can be used for tolerance applications in transplantation and in auto-immunity. Currently, T_{reg} infusions are expanded ex vivo. Phase I studies have shown marginal, if any, efficacy in T1D, and in some cases there have been benefits in post-transplant GVHD. For next generation engineered regulatory T cells, in some embodiments, these can be chimeric antigen receptor (CAR) directed natural T_{regs}. Effector T cells can also be converted to T_{regs} by FOXP3 expression.

However, there may also be differences between engineered versus natural T_{regs} for methods of treatment. Natural T_{reg} therapy has been considered safe, however too few natural T_{regs} causes autoimmunity. T_{reg} are believed to play a critical role in multiple autoimmune diseases, such as IPEX syndrome, Type 1 diabetes, systemic lupus erythematosus, and rheumatoid arthritis. Approaches to augment human T_{reg} number or function are in current trials, including low-dose IL-2 and adoptive transfer of autologous expanded T_{reg}. The efficacy of IL-2 therapy is limited due to its pleotropic activity and potential “off target” effects that may increase inflammation. Adoptive T_{reg} therapy is likely limited by in vivo stability and viability of expanded T_{regs}, and their lack of relevant antigen specificity.

There are also potential flaws with the use of natural T_{regs}. For example, autoimmune patients are genetically predisposed to T_{reg} instability. For example, it is plausible for a CAR-bearing nT_{reg} to convert to a CAR T effector cell. nT_{reg} cells also retain the potential for epigenetic regulation of FOXP3, which could lead to the down regulation of FOXP3 induction, which means that the function of an nT_{reg} population may never be fully predictable. Also, natural T_{regs} may not include the correct TCR (T cell receptor) specificities. The T_{reg} function may also be linked to a selectable marker in which the expanded native T_{reg} cell population may always have contaminating inflammatory cells. Thus, the methods provided herein are an improvement over using the transfer of natural T_{regs} by using engineered cells, as there is potential for linking CAR expression to regulatory T cell function to avoid potential engraftment of CAR T_{regs} that have the potential to convert to pro inflammatory CAR T cells.

Thymus-derived regulatory T cells (tT_{reg} or nT_{reg}) stably express FOXP3 which plays a crucial role in the suppressive function of T_{reg}. In the exemplary studies described herein, it was shown that stable expression of FOXP3 through knocking in a constitutive promoter upstream of FOXP3 gene acquires CD4+T_{conv} cells suppressive function that is similar to tT_{reg}. This has also been described PCT/US2016/059729 (included by reference in its entirety herein).

The approach to drive endogenous FOXP3 expression restricts the editing to FOXP3 locus, and may not be suitable for donors that carry FOXP3 mutations (see, e.g., Example 1). To further broaden the applications of this technique, mRNA of FOXP3 was expressed by introducing a promoter and a codon-optimized FOXP3 cDNA sequence in either a FOXP3 or non-FOXP3 locus. Using selection markers, for example, LNGFR and DISC/pDISC, can enable enrichment of the cell products.

### Definitions

As used herein, “nucleic acid” or “nucleic acid molecule” includes but is not limited to, for example, polynucleotides or oligonucleotides such as deoxyribonucleic acid (DNA) or ribonucleic acid (RNA), oligonucleotides, fragments generated by the polymerase chain reaction (PCR), and fragments generated by any of ligation, scission, endonuclease action, exonuclease action, and by synthetic generation. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, or phosphoramidate. The term “nucleic acid molecule” also includes so-called “peptide nucleic acids,” which comprise naturally-occurring or modified nucleic acid bases attached to a polyamide backbone. Nucleic acids can be either single stranded or double stranded.

“Coding strand” includes but is not limited to, for example, the DNA strand which has the same base sequence as the RNA transcript produced (although with thymine replaced by uracil). It is this strand, which contains codons, while the non-coding strand contains anti-codons.

“Regulatory element” includes but is not limited to, for example, a segment of a nucleic acid molecule, which is capable of increasing or decreasing the expression of specific genes within an organism, e.g., one that has the ability to affect the transcription and/or translation of an operably linked transcribable DNA molecule. Regulatory elements such as promoters (e.g. an MND promoter), leaders, introns, and transcription termination regions are DNA molecules that have gene regulatory activity and play an integral part in the overall expression of genes in living cells. Isolated regulatory elements, such as promoters, that function in plants are therefore useful for modifying plant phenotypes through the methods of genetic engineering. Regulation of gene expression is an essential feature of all living organisms and viruses. Without limitation, examples of regulatory elements can include, CAAT box, CCAAT box, Pribnow box, TATA box, SECIS element, mRNA Polyadenylation signals, A-box, Z-box, C-box, E-box, G-box, hormone responsive elements, such as insulin gene regulatory sequences, DNA binding domains, activation domains, and/or enhancer domains.

In some embodiments, a guide RNA includes an additional segment at either the 5′ or 3′ end that provides for any of the features described above. For example, a suitable third segment can include a 5′ cap (e.g. a 7-methylguanylate cap (m7G)); a 3′ polyadenylated tail (e.g., a 3′ poly(A) tail); a riboswitch sequence (e.g. to allow for regulated stability and/or regulated accessibility by proteins and protein complexes); a stability control sequence; a sequence that forms a dsRNA duplex (e.g., a hairpin)); a sequence that targets the RNA to a subcellular location (e.g., nucleus, mitochondria, chloroplasts, and the like); a modification or sequence that provides for tracking (e.g. direct conjugation to a fluorescent molecule, conjugation to a moiety that facilitates fluorescent detection, a sequence that allows for fluorescent detection, etc.); a modification or sequence that provides a binding site for proteins (e.g., proteins that act on DNA. including transcriptional activators, transcriptional repressors, DNA methyltransferases, DNA demethylases, histone acetyltransferases, histone deacetylases, and the like); and combinations thereof.

A guide RNA and a Cas protein may form a ribonucleoprotein complex (e.g., bind via non-covalent interactions). The guide RNA provides target specificity to the complex by comprising a nucleotide sequence that is complementary to a sequence of a target DNA. The site-specific modifying enzyme of the complex provides the endonuclease activity. In other words, the site-specific modifying enzyme is guided to a target DNA sequence (e.g. a target sequence in a chromosomal nucleic acid; a target sequence in an extrachromosomal nucleic acid, e.g. an episomal nucleic acid, a minicircle, etc.; a target sequence in a mitochondrial nucleic acid; a target sequence in a chloroplast nucleic acid; a target sequence in a plasmid; etc.) by virtue of its association with the protein-binding segment of the guide RNA.

“FOXP3” as used herein includes but is not limited to, for example, a protein that is involved in immune system responses. The FOXP3 gene contains 11 coding exons. FOXP3 is a specific marker of natural T regulatory cells (nT_{regs}, a lineage of T cells) and adaptive/induced T regulatory cells (a/iT_{regs}). Induction or administration of FOXP3 positive T cells in animal studies was shown to lead to marked reductions in (autoimmune) disease severity in models of diabetes, multiple sclerosis, asthma, inflammatory bowel disease, thyroiditis and renal disease. However, T cells have been able to show plasticity. Thus, the use of regulatory T cells in therapy can be complicated, as the T regulatory cell transferred to the subject may change into T helper 17 (Th17) cells, which are pro-inflammatory, rather than regulatory cells. As such, methods are provided herein to avoid the complications that may arise from regulatory cells changing into pro-inflammatory cells. For example, FOXP3 expressed from an iT_{reg} is used as a master regulator of the immune system, and is used for tolerance and immune suppression. T_{reg} are believed to play a critical role in multiple autoimmune diseases, such as IPEX syndrome, Type 1 diabetes, systemic lupus erythematosus, and rheumatoid arthritis. Approaches to augment human T_{reg} number or function are in current trials, including low-dose IL-2 and adoptive transfer of autologous expanded T_{reg}. The efficacy of IL-2 therapy is limited due to its pleotropic activity and potential “off target” effects that may increase inflammation. Adoptive T_{reg} therapy is likely limited by in vivo stability and viability of expanded T_{regs}, and their lack of relevant antigen specificity.

“Nuclease” includes but is not limited to, for example, a protein or an enzyme capable of cleaving the phosphodiester bonds between the nucleotide subunits of nucleic acids. The nuclease described herein is used for “gene editing”, which is a type of genetic engineering in which DNA is inserted, deleted or replaced in the genome of a living organism, using a nuclease or an engineered nuclease or nucleases. Without limitation, the nuclease can be of the CRISPR/CAS system, a zinc finger nuclease, or a TALEN nuclease. The nuclease can be used to target a locus, or a specific nucleic acid sequence.

“Coding exon” includes but is not limited to, for example, any part of a gene that will encode a part of the final mature RNA produced by that gene after introns have been removed by RNA splicing. The term “exon” refers to both the DNA sequence within a gene and to the corresponding sequence in RNA transcripts. In RNA splicing, introns are removed and exons are covalently joined to one another as part of generating the mature messenger RNA.

“Cas endonuclease” or “Cas nuclease” as used herein includes without limitation, for example, an RNA-guided DNA endonuclease enzyme associated with a CRISPR (Clustered Regularly Interspaced Short Palindromic Repeats) adaptive immunity system. Herein, “Cas endonuclease” refers to both naturally-occurring and recombinant Cas endonucleases. “Cas9” includes but is not limited to, for example, an RNA-guided DNA endonuclease enzyme associated with the CRISPR (Clustered Regularly Interspaced Short Palindromic Repeats) adaptive immunity system.

“Zinc finger nuclease” as used herein includes but is not limited to, for example, an artificial restriction enzymes generated by fusing a zinc finger DNA-binding domain to a DNA-cleavage domain. Zinc finger domains can be engineered to target specific desired DNA sequences and this enables zinc-finger nucleases to target unique sequences within complex genomes.

“TALEN” or “Transcription activator-like effector nuclease” as used herein include, but are not limited to, for example, restriction enzymes that can be engineered to cut specific sequences of DNA. They are made by fusing a TAL effector DNA-binding domain to a DNA cleavage domain (a nuclease which cuts DNA strands). Transcription activator-like effectors (TALEs) can be engineered to bind practically any desired DNA sequence, so when combined with a nuclease, DNA can be cut at specific locations. The restriction enzymes can be introduced into cells, for use in gene editing or for genome editing in situ, a technique known as genome editing with engineered nucleases. Alongside zinc finger nucleases and CRISPR/Cas9, TALEN is a prominent tool in the field of genome editing.

“Knock-in” includes but is not limited to, for example, a genetic engineering method that involves the one-for-one substitution of DNA sequence information with a different copy in a genetic locus or the insertion of sequence information not found within the locus.

A “promoter” includes but is not limited to, for example, a nucleotide sequence that directs the transcription of a structural gene. In some embodiments, a promoter is located in the 5′ non-coding region of a gene, proximal to the transcriptional start site of a structural gene. Sequence elements within promoters that function in the initiation of transcription are often characterized by consensus nucleotide sequences. It is a region of DNA that initiates transcription of a particular gene. Promoters are located near the transcription start sites of genes, on the same strand and upstream on the DNA (towards the 5′ region of the sense strand). Promoters can be at or about 100, 200, 300, 400, 500, 600, 700, 800, or 1000 base pairs long, or within a range defined by any two of the aforementioned lengths. As used herein, a promoter can be constitutively active, repressible or inducible. If a promoter is an inducible promoter, then the rate of transcription increases in response to an inducing agent. In contrast, the rate of transcription is not regulated by an inducing agent if the promoter is a constitutive promoter. Repressible promoters are also known. Without limitation, examples of promoters can include a constitutive promoter, a heterologous weak promoter (e.g., a promoter that generates less expression than the endogenous promoter and/or a constitutive promoter), and inducible promoters. Examples can include an EF1 alpha promoter, a PGK promoter, an MND promoter, a KI promoter, a Ki-67 gene promoter, and/or a promoter inducible by a drug such as tamoxifen and/or its metabolites. Commonly used constitutive promoters can include but are not limited to SV40, CMV, UBC, EF1A, PGK, and/or CAGG for mammalian systems.

A weak promoter produces less mRNA expression than a stronger promoter, if both are driving expression of the same coding sequences. This can be compared by analyzing, for example, an agarose gel. An example of promoters subject to regulation by proximal chromatin is the EF1alpha short promoter, which is highly active in some loci, but nearly inactive in other loci (Eyquem, J. et al. (2013). Biotechnol. Bioeng., 110(8):2225-2235).

“Transcriptional enhancer domain” includes but is not limited to, for example, a short (50-1500 bp) region of DNA that can be bound by proteins (activators) to increase or promote or enhance the likelihood that transcription of a particular gene will occur or the level of transcription that takes place. These activator proteins are usually referred to as transcription factors. Enhancers are generally cis-acting, located up to 1 Mbp (1,000,000 bp) away from the gene, and can be upstream or downstream from the start site, and either in the forward or backward direction. An enhancer may be located upstream or downstream of the gene it regulates. A plurality of enhancer domains may be used in some embodiments to generate greater transcription, e.g., multimerized activation binding domains can be used to further enhance or increase the level of transcription. Furthermore, an enhancer does not need to be located near the transcription initiation site to affect transcription, as some have been found located several hundred thousand base pairs upstream or downstream of the start site. Enhancers do not act on the promoter region itself, but are bound by activator proteins. These activator proteins interact with the mediator complex, which recruits polymerase II and the general transcription factors, which then begin transcribing the genes. Enhancers can also be found within introns. An enhancer's orientation may even be reversed without affecting its function. Additionally, an enhancer may be excised and inserted elsewhere in the chromosome, and still affect gene transcription. In some embodiments, enhancers are used to silence the inhibition mechanisms that prevent transcription of the FOXP3 gene. An example of an enhancer binding domain is the TCR alpha enhancer. In some embodiments, the enhancer domain in the embodiments described herein is a TCR alpha enhancer. In some embodiments, the enhancer binding domain is placed upstream from a promoter such that it activates the promoter to increase transcription of the protein. In some embodiments, the enhancer binding domain is placed upstream of a promoter to activate the promoter to increase transcription of the FOXP3 gene.

“Transcriptional activation domain” includes but is not limited to, for example, specific DNA sequences that can be bound by a transcription factor, in which the transcription factor can thereby control the rate of transcription of genetic information from DNA to messenger RNA. Specific transcription factors can include but are not limited to SP1, AP1, C/EBP, heat shock factor, ATF/CREB, c-Myc, Oct-1 and/or NF-1. In some embodiments, the activator domains are used to silence the inhibition mechanisms that prevent transcription of the FOXP3 gene.

“Ubiquitous chromatin opening element” (UCOE) includes but is not limited to, for example, elements that are characterized by unmethylated CpG islands spanning dual, divergently transcribed promoters of housekeeping genes. The UCOE represent promising tools to avoid silencing and sustain transgene expression in a wide variety of cellular models including cell lines, multipotent hematopoietic stem cells, as well as PSCs and their differentiated progeny. “Operably linked” includes but is not limited to, for example, functional linkage between a regulatory sequence and a heterologous nucleic acid sequence resulting in expression of the latter. In some embodiments, the first molecule is joined to a second molecule, wherein the molecules are so arranged that the first molecule affects the function of the second molecule. The two molecules may be part of a single contiguous molecule and may be adjacent. For example, a promoter is operably linked to a transcribable DNA molecule if the promoter modulates transcription of the transcribable DNA molecule of interest in a cell.

The term “concentration” used in the context of a molecule such as peptide fragment refers to an amount of molecule, e.g., the number of moles of the molecule, present in a given volume of solution.

The terms “individual,” “subject”, and “host” are used interchangeably herein and refer to any subject for whom diagnosis, treatment, or therapy is desired. In some aspects, the subject is a mammal. In some aspects, the subject is a human being. In some aspects, the subject is a human patient. In some aspects, the subject can have or is suspected of having a disorder or health condition associated with FOXP3. In some aspects, the subject is a human who is diagnosed with a risk of disorder or health condition associated with FOXP3 at the time of diagnosis or later. In some cases, the diagnosis with a risk of disorder or health condition associated with FOXP3 can be determined based on the presence of one or more mutations in an endogenous gene encoding the FOXP3 or nearby genomic sequence that may affect the expression of FOXP3. For example, in some aspects, the subject can have or is suspected of having an autoimmune disorder and/or has one or more symptoms of an autoimmune disorder. In some aspects, the subject is a human who is diagnosed with a risk of an autoimmune disorder at the time of diagnosis or later. In some cases, the diagnosis with a risk of an autoimmune disorder can be determined based on the presence of one or more mutations in an endogenous FOXP3 gene or genomic sequence near the FOXP3 gene in the genome that may affect the expression of the FOXP3 gene.

The term “treatment,” when used in referring to a disease or condition, means that at least an amelioration of the symptoms associated with the condition afflicting an individual is achieved, where amelioration is used in a broad sense to refer to at least a reduction in the magnitude of a parameter, e.g., a symptom, associated with the condition (e.g., an autoimmune disorder) being treated. As such, treatment also includes situations where the pathological condition, or at least symptoms associated therewith, are completely inhibited, e.g., prevented from happening, or eliminated entirely such that the host no longer suffers from the condition, or at least the symptoms that characterize the condition. Thus, treatment includes: (i) prevention, that is, reducing the risk of development of clinical symptoms, including causing the clinical symptoms not to develop, e.g., preventing disease progression; and (ii) inhibition, that is, arresting the development or further development of clinical symptoms, e.g., mitigating or completely inhibiting an active disease.

The terms “effective amount,” “pharmaceutically effective amount,” and “therapeutically effective amount”, as used herein mean a sufficient amount of the composition to provide the desired utility when administered to a subject having a particular condition. In the context of ex vivo treatment of an autoimmune disorder, the term “effective amount” refers to the amount of a population of therapeutic cells or their progeny needed to prevent or alleviate at least one or more signs or symptoms of an autoimmune disorder, and relates to a sufficient amount of a composition having the therapeutic cells or their progeny to provide the desired effect, e.g., to treat symptoms of an autoimmune disorder of a subject. The term “therapeutically effective amount” therefore refers to a number of therapeutic cells, or a composition having therapeutic cells, that is sufficient to promote a particular effect when administered to a subject in need of treatment, such as one who has or is at risk for an autoimmune disorder. An effective amount would also include an amount sufficient to prevent or delay the development of a symptom of the disease, alter the course of a symptom of the disease (for example but not limited to, slow the progression of a symptom of the disease), or reverse a symptom of the disease. In the context of in vivo treatment of an autoimmune disorder in a subject (e.g., a patient) or genome edition in a cell cultured in vitro, an effective amount refers to an amount of components used for genome edition such as gRNA, donor template and/or a site-directed polypeptide (e.g. DNA endonuclease) needed to edit the genome of the cell in the subject or the cell cultured in vitro. It is understood that for any given case, an appropriate “effective amount” can be determined by one of ordinary skill in the art using routine experimentation.

“Autoimmune disorder” includes but is not limited to, for example, abnormally low activity or overactivity of the immune system. In cases of immune system overactivity, the body attacks and damages its own tissues (autoimmune diseases). Immune deficiency diseases decrease the body's ability to fight invaders, causing vulnerability to infections. Without being limiting, examples of autoimmune disorders or autoimmune diseases can include, for example, systemic lupus, scleroderma, hemolytic anemia, vasculitis, type I diabetes, Graves disease, rheumatoid arthritis, multiple sclerosis, Goodpasture's syndrome, myopathy, severe combined immunodeficiency, DiGeorge syndrome, Hyperimmunoglobulin E syndrome, Common variable immunodeficiency, Chronic granulomatous disease, Wiskott-Aldrich syndrome, Autoimmune lymphoproliferative syndrome, Hyper IgM syndrome, Leukocyte adhesion deficiency, NF-κB Essential Modifier (NEMO) Mutations, Selective immunoglobulin A deficiency, X-linked agammaglobulinemia, X-linked lymphoproliferative disease, IPEX, Immune dysregulation, polyendocrinopathy, enteropathy, X-linked (IPEX) syndrome and/or Ataxia-telangiectasia. Immune disorders can be analyzed, for example, by examination of the profile of neural-specific autoantibodies or other biomarkers when detected in serum or cerebrospinal fluid in subjects. In some embodiment methods provided herein, the methods are for treatment, amelioration, or inhibition of autoimmune disorders. In some embodiments, the autoimmune disorder is systemic lupus, scleroderma, hemolytic anemia, vasculitis, type I diabetes, Graves disease, rheumatoid arthritis, multiple sclerosis, Goodpasture's syndrome, myopathy, severe combined immunodeficiency, DiGeorge syndrome, Hyperimmunoglobulin E syndrome, Common variable immunodeficiency, Chronic granulomatous disease, Wiskott-Aldrich syndrome, Autoimmune lymphoproliferative syndrome, Hyper IgM syndrome, Leukocyte adhesion deficiency, NF-κB Essential Modifier (NEMO) Mutations, Selective immunoglobulin A deficiency, X-linked agammaglobulinemia, X-linked lymphoproliferative disease, IPEX, Immune dysregulation, polyendocrinopathy, enteropathy, X-linked (IPEX) syndrome, and/or Ataxia-telangiectasia.

“IPEX syndrome” refers to immunodysregulation polyendocrinopathy enteropathy X-linked syndrome, a rare disease linked to dysfunction of FOXP3, widely considered to be a master regulator of the regulatory T cell lineage. Subjects suffering from IPEX syndrome may have symptoms such as autoimmune enteropathy, psoriasiform or eczematous dermatitis, nail dystrophy, autoimmune endocrinopathies, and/or autoimmune skin conditions such as alopecia universalis and/or bullous pemphigoid. IPEX is an autoimmune disease in which the immune system attacks the body's own tissues and organs. The syndrome leads to loss of CD4+CD25+T regulatory cells, and loss of the expression of transcription factor FOXP3. FOXP3 decrease is believed to be a consequence of unchecked T cell activation, which is secondary to loss of regulatory T cells.

“Organ transplantation” includes but is not limited to, for example, the moving of an organ from one body to another or from a donor site to another location on the person's own body, to replace the recipient's damaged or absent organ. Organs and/or tissues that are transplanted within the same person's body are called autografts. Transplants that are recently performed between two subjects of the same species are called allografts. Allografts can either be from a living or cadaveric source. In some embodiments described herein, a method of treating, inhibiting, or ameliorating side effects of organ transplantation in a subject, such as organ rejection is provided.

Organs that can be transplanted, for example, are the heart, kidneys, liver, lungs, pancreas, intestine, and/or thymus. Tissues for transplant can include, for example, bones, tendons (both referred to as musculoskeletal grafts), cornea, skin, heart valves, nerves and/or veins. Kidneys, liver and the heart are the most commonly transplanted organs. Cornea and musculoskeletal grafts are the most commonly transplanted tissues.

In some embodiments described herein, a method of treating, inhibiting, or ameliorating side effects of organ transplantation in a subject, such as organ rejection is provided. In some embodiments, the subject is also selected or identified to receive one or more anti-rejection medications. In some embodiments, the anti-rejection medications comprise Prednisone, Imuran (azathioprine), Collect (mycophenolate mofetil, or MMF), Myfortic (mycophenolic acid), Rapamune (sirolimus), Neoral (cyclosporine), and/or Prograf (tacrolimus).

In some embodiments, the subject is selected for inhibition, amelioration, or treatment with the engineered cells of the embodiments herein. In some embodiments, the subject has experienced one or more side effects to anti-inflammatory drugs or anti-rejection drugs. As such, the selected subjects are provided with the exemplary cells or compositions provided herein. Side effects from anti-rejection drugs can include interactions with other medications that can raise or lower tacrolimus levels in the blood, kidney toxicity, high blood pressure, neurotoxicity (tremor, headache, tingling, and insomnia), Diabetes mellitis (high blood sugar), diarrhea, nausea, hair loss and/or high potassium. As such, the subjects are selected for the methods of treatment, inhibition, or amelioration described herein by clinical or diagnostic evaluation.

“Organ rejection” or “transplant rejection” as used herein includes but is not limited to, for example, transplanted tissue rejected by the recipient's immune system, which destroys the transplanted tissue.

“Graft-versus-host disease” (GVHD) includes but is not limited to, for example, a medical complication following the receipt of transplanted tissue from a genetically different person. GVHD is commonly associated with stem cell or bone marrow transplant but the term also applies to other forms of tissue graft. Immune cells in the donated tissue recognize the recipient as foreign and not “self.” In some embodiments herein, the methods provided can be used for preventing or ameliorating the complications that can arise from GVHD.

“Pharmaceutical excipient” includes but is not limited to, for example, the inert substance that the cells in the composition are provided in.

A “chimeric antigen receptor” (CAR) described herein, also known as chimeric T cell receptor, includes but is not limited to, for example, an artificial T cell receptor or a genetically engineered receptor, which grafts a desired specificity onto an immune effector cell. A CAR may be a synthetically designed receptor comprising a ligand binding domain of an antibody or other protein sequence that binds to a molecule associated with the disease or disorder and is linked via a spacer domain to one or more intracellular signaling domains of a T cell or other receptors, such as a costimulatory domain. In some embodiments, a cell, such as a mammalian cell, is manufactured wherein the cell comprises a nucleic acid encoding a fusion protein and wherein the cell comprises a chimeric antigen receptor. These receptors can be used to graft the specificity of a monoclonal antibody or a binding portion thereof onto a T cell, for example. In some embodiments herein, the genetically engineered cell further comprises a sequence that encodes a chimeric antigen receptor. In some embodiments, the chimeric antigen receptor is specific for a molecule on a tumor cell. A chimeric antigen receptor or an engineered cell expressing a T cell receptor can be used to target a specific tissue in need for FOXP3. In some embodiments herein comprise methods for targeting specific tissues for providing and delivering FOXP3. In some embodiments, the tissue is a transplanted tissue. In some embodiments, the chimeric antigen receptor is specific for a target molecule on the transplanted tissue.

As described herein, the genetically-engineered cells are engineered to express FOXP3, and as such, they are also described in the embodiments herein as “T_{reg}-phenotype” cells.

As used herein, “protein sequence” includes but is not limited to, for example, a polypeptide sequence of amino acids that is the primary structure of a protein. As used herein “upstream” refers to positions 5′ of a location on a polynucleotide, and positions toward the N-terminus of a location on a polypeptide. As used herein “downstream” refers to positions 3′ of a location on nucleotide, and positions toward the C-terminus of a location on a polypeptide. Thus, the term “N-terminal” refers to the position of an element or location on a polynucleotide toward the N-terminus of a location on a polypeptide.

The functional equivalent or fragment of the functional equivalent, in the context of a protein, may have one or more conservative amino acid substitutions. The term “conservative amino acid substitution” refers to substitution of an amino acid for another amino acid that has similar properties as the original amino acid. The groups of conservative amino acids are as follows:

| Group | Name of the amino acids |
|---|---|
| Aliphatic | Gly, Ala, Val, Leu, Ile |
| Hydroxyl or Sulfhydryl/Selenium-containing | Ser, Cys, Thr, Met |
| Cyclic | Pro |
| Aromatic | Phe, Tyr, Trp |
| Basic | His, Lys, Arg |
| Acidic and their Amide | Asp, Glu, Asn, Gln |

Conservative substitutions may be introduced in any position of a predetermined peptide or fragment thereof. It may however also be desirable to introduce non-conservative substitutions, particularly, but not limited to, a non-conservative substitution in any one or more positions. A non-conservative substitution leading to the formation of a functionally equivalent fragment of the peptide would for example differ substantially in polarity, in electric charge, and/or in steric bulk while maintaining the functionality of the derivative or variant fragment.

“Percentage of sequence identity” is determined by comparing two optimally aligned sequences over a comparison window, wherein the portion of the polynucleotide or polypeptide sequence in the comparison window may have additions or deletions (such as gaps) as compared to the reference sequence (which does not have additions or deletions) for optimal alignment of the two sequences. In some cases, the percentage can be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity” in the context of two or more nucleic acid or polypeptide sequences, refer to two or more sequences or subsequences that are the same or have a specified percentage of amino acid residues or nucleotides that are the same (e.g., 60%, 65%, 70%, 75%, 80%, 85%, 90%, 95%, 98%, or 99% identity over a specified region, e.g., the entire polypeptide sequences or individual domains of the polypeptides), when compared and aligned for maximum correspondence over a comparison window or designated region as measured using one of the following sequence comparison algorithms or by manual alignment and visual inspection. Such sequences are then said to be “substantially identical.” This definition also refers to the complement of a test sequence.

The term “complementary” or “substantially complementary,” interchangeably used herein, means that a nucleic acid (e.g., DNA or RNA) has a sequence of nucleotides that enables it to non-covalently bind, such as form Watson-Crick base pairs and/or G/U base pairs, to another nucleic acid in a sequence-specific, antiparallel, manner (such as a nucleic acid specifically binds to a complementary nucleic acid). As is known in the art, standard Watson-Crick base-pairing includes: adenine (A) pairing with thymidine (T), adenine (A) pairing with uracil (U), and guanine (G) pairing with cytosine (C).

A DNA sequence that “encodes” a particular RNA is a DNA nucleic acid sequence that can be transcribed into RNA. A DNA polynucleotide may encode an RNA (mRNA) that is translated into protein, or a DNA polynucleotide may encode an RNA that is not translated into protein (e.g., tRNA, rRNA, or a guide RNA; also referred to herein as “non-coding” RNA or “ncRNA”). A “protein coding sequence or a sequence that encodes a particular protein or polypeptide, is a nucleic acid sequence that is transcribed into mRNA (in the case of DNA) and is translated (in the case of mRNA) into a polypeptide in vitro or in vivo when placed under the control of appropriate regulatory sequences.

As used herein, “codon” refers to a sequence of three nucleotides that together form a unit of genetic code in a DNA or RNA molecule. As used herein the term “codon degeneracy” refers to the nature in the genetic code permitting variation of the nucleotide sequence without affecting the amino acid sequence of an encoded polypeptide.

The term “codon-optimized” or “codon optimization” refers to genes or coding regions of nucleic acid molecules for transformation of various hosts, refers to the alteration of codons in the gene or coding regions of the nucleic acid molecules to reflect the typical codon usage of the host organism without altering the polypeptide encoded by the DNA. Such optimization includes replacing at least one, or more than one, or a significant number, of codons with one or more codons that are more frequently used in the genes of that organism. Codon usage tables are readily available, for example, at the “Codon Usage Database” available at www.kazusa.or.jp/codon/(visited Mar. 20, 2019). By utilizing the knowledge on codon usage or codon preference in each organism, one of ordinary skill in the art can apply the frequencies to any given polypeptide sequence and produce a nucleic acid fragment of a codon-optimized coding region which encodes the polypeptide, but which uses codons optimal for a given species. Codon-optimized coding regions can be designed by various methods known to those skilled in the art.

The term “recombinant” or “engineered” when used with reference, for example, to a cell, a nucleic acid, a protein, or a vector, indicates that the cell, nucleic acid, protein, or vector has been modified by or is the result of laboratory methods. Thus, for example, recombinant or engineered proteins include proteins produced by laboratory methods. Recombinant or engineered proteins can include amino acid residues not found within the native (non-recombinant or wild-type) form of the protein or can be include amino acid residues that have been modified, e.g., labeled. The term can include any modifications to the peptide, protein, or nucleic acid sequence. Such modifications may include the following: any chemical modifications of the peptide, protein, or nucleic acid sequence, including of one or more amino acids, deoxyribonucleotides, or ribonucleotides; addition, deletion, and/or substitution of one or more of amino acids in the peptide or protein; and addition, deletion, and/or substitution of one or more of nucleic acids in the nucleic acid sequence.

The term “genomic DNA” or “genomic sequence” refers to the DNA of a genome of an organism including, but not limited to, the DNA of the genome of a bacterium, fungus, archaeon, plant, or animal.

As used herein, “transgene,” “exogenous gene” or “exogenous sequence,” in the context of nucleic acid, refers to a nucleic acid sequence or gene that was not present in the genome of a cell but artificially introduced into the genome, e.g., via genome-edition.

As used herein, “endogenous gene” or “endogenous sequence,” in the context of nucleic acid, refers to a nucleic acid sequence or gene that is naturally present in the genome of a cell, without being introduced via any artificial means.

As used herein, the term “expression,” or “protein expression” refers to the translation of a transcribed RNA molecule into a protein molecule. Protein expression may be characterized by its temporal, spatial, developmental, or morphological qualities as well as by quantitative or qualitative indications. In some embodiments, the protein or proteins are expressed such that the proteins are positioned for dimerization in the presence of a ligand.

As used herein, “fusion proteins” or “chimeric proteins” are proteins created through the joining of two or more genes that originally coded for separate proteins or portions of proteins. The fusion proteins can also be made up of specific protein domains from two or more separate proteins. Translation of this fusion gene can result in a single or multiple polypeptides with functional properties derived from each of the original proteins. Recombinant fusion proteins can be created artificially by recombinant DNA technology for use in biological research or therapeutics. Such methods for creating fusion proteins are known to those skilled in the art. Some fusion proteins combine whole peptides and therefore can contain all domains, especially functional domains, of the original proteins. However, other fusion proteins, especially those that are non-naturally occurring, combine only portions of coding sequences and therefore do not maintain the original functions of the parental genes that formed them.

“Vector,” “expression vector,” or “construct” is a nucleic acid used to introduce heterologous nucleic acids into a cell that has regulatory elements to provide expression of the heterologous nucleic acids in the cell. Vectors include but are not limited to plasmid, minicircles, yeast, and viral genomes. In some embodiments, the vectors are plasmid, minicircles, yeast, or viral genomes. In some embodiments, the vector is a viral vector. In some embodiments, the viral vector is a lentivirus. In some embodiments, the vector is an adeno-associated viral (AAV) vector. In some embodiments, the vector is for protein expression in a bacterial system such as E. coli. As used herein, the term “expression,” or “protein expression” refers to refers to the translation of a transcribed RNA molecule into a protein molecule. Protein expression may be characterized by its temporal, spatial, developmental, or morphological qualities, as well as, by quantitative or qualitative indications. In some embodiments, the protein or proteins are expressed such that the proteins are positioned for dimerization in the presence of a ligand. In some embodiments, the vector is a viral vector. In some embodiments, the viral vector is a lentivirus. In some embodiments, the vector is an adeno-associated viral (AAV) vector (such as, without limitation, AAV1, AAV2, AAV3, AAV4, AAV5, AAV6, AAV7, AAV8, AAV9, AAV10, or AAV11).

As used herein, “fusion proteins” or “chimeric proteins” includes but is not limited to, for example, proteins created through the joining of two or more genes that originally coded for separate proteins or portions of proteins. The fusion proteins can also be made up of specific protein domains from two or more separate proteins. Translation of this fusion gene can result in a single or multiple polypeptides with functional properties derived from each of the original proteins. Recombinant fusion proteins can be created artificially by recombinant DNA technology for use in biological research or therapeutics. Such methods for creating fusion proteins are known to those skilled in the art. Some fusion proteins combine whole peptides and therefore can contain all domains, especially functional domains, of the original proteins. However, other fusion proteins, especially those that are non-naturally occurring, combine only portions of coding sequences and therefore do not maintain the original functions of the parental genes that formed them. In some embodiments, a fusion protein is provided, wherein the fusion protein comprises an interferon and/or a PD-1 protein.

“Conditional” or “inducible” promoter includes but is not limited to, for example, a nucleic acid construct that comprises a promoter that provides for gene expression in the presence of an inducer and does not substantially provide for gene expression in the absence of the inducer.

“Constitutive” as used herein refer to the nucleic acid construct that comprises a promoter that is constitutive, and thus provides for expression of a polypeptide that is continuously produced.

In some embodiments, the inducible promoter has a low level of basal activity. In some embodiments, wherein a lentiviral vector is used, the level of basal activity in uninduced cells is 20%, 15%, 10%, 5%, 4%, 3%, 2%, 1% or less (but not zero) or within a range defined by any two of the aforementioned values, as compared to when cells are induced to express the gene. The level of basal activity can be determined by measuring the amount of the expression of the transgene (e.g. marker gene) in the absence of the inducer (e.g. drug) using flow cytometry. In some embodiments described herein a marker protein such as Akt is used for determination of expression.

In some embodiments, the inducible promoter provides for a high level of induced activity, as compared to uninduced or basal activity. In some embodiments, the level of activity in the induced state is 2, 4, 6, 8, 9 or 10 fold or greater than the activity level in the uninduced state or within a range defined by any two of the aforementioned values. In some embodiments, transgene expression under control of the inducible promoter is turned off in the absence of a transactivator in less than 10, 8, 6, 4, 2, or 1 days excluding 0 days or within a range defined by any two of the aforementioned time periods.

In some embodiments, an inducible promoter is designed and/or modified to provide for a low level of basal activity, a high level of inducibility, and/or a short time for reversibility.

“Dimeric chemical-induced signaling complex,” “dimeric CISC,” or “dimer” as used herein refers to two components of a CISC, which may or may not be fusion protein complexes that join together. “Dimerization” refers to the process of the joining together of two separate entities into a single entity. In some embodiments, a ligand or agent stimulates dimerization. In some embodiments, dimerization refers to homodimerization, or the joining of two identical entities, such as two identical CISC components. In some embodiments, dimerization refers to heterodimerization, of the joining of two different entities, such as two different and distinct CISC components. In some embodiments, the dimerization of the CISC components results in a cellular signaling pathway. In some embodiments, the dimerization of the CISC components allows for the selective expansion of a cell or a population of cells. Additional CISC systems can include a CISC gibberellin CISC dimerization system, or a SLF-TMP CISC dimerization system. Other chemically inducible dimerization (CID) systems and component parts may be used.

As used herein, “chemical-induced signaling complex” or “CISC” refers to an engineered complex that initiates a signal into the interior of a cell as a direct outcome of ligand-induced dimerization. A CISC may be a homodimer (dimerization of two identical components) or a heterodimer (dimerization of two distinct components). Thus, as used herein the term “homodimer” refers to a dimer of two protein components described herein with identical amino acid sequences. The term “heterodimer” refers to a dimer of two protein components described herein with non-identical amino acid sequences.

The CISC may be a synthetic complex as described herein in greater detail. “Synthetic” as used herein refers to a complex, protein, dimer, or composition, as described herein, which is not natural, or that is not found in nature. In some embodiments, an IL2R-CISC refers to a signaling complex that involves interleukin-2 receptor components. In some embodiments, an IL2/15-CISC refers to a signaling complex that involves receptor signaling subunits that are shared by interleukin-2 and/or interleukin-15. In some embodiments, an IL7-CISC refers to a signaling complex that involves an interleukin-7 receptor components. A CISC may thus be termed according to the component parts that make up the components of a given CISC. One of skill in the art will recognize that the component parts of the chemical-induced signaling complex may be composed of a natural or a synthetic component useful for incorporation into a CISC. Thus, the examples provided herein are not intended to be limiting.

The CISC (chemically induced signaling complex) is a multicomponent synthetic protein complex configured for co-expression in a host cell as two chimeric proteins as described in International Patent Application No. PCT/US2017/065746, the disclosure of which is incorporated by reference herein in its entirety. Each chimeric protein component of the CISC has one half of a rapamycin binding complex as an extracellular domain, fused to one half of an intracellular signaling complex. Delivery of nucleic acids encoding the CISC to host cells permits intracellular signaling in the cells that can be controlled by the presence of rapamycin or a rapamycin-related chemical compound.

As used herein, “cytokine receptor” refers to receptor molecules that recognize and bind to cytokines. In some embodiments, cytokine receptor encompasses modified cytokine receptor molecules (e.g., “variant cytokine receptors”), comprising those with substitutions, deletions, and/or additions to the cytokine receptor amino acid and/or nucleic acid sequence. Thus, it is intended that the term encompass wild-type, as well as, recombinant, synthetically-produced, and variant cytokine receptors. In some embodiments, the cytokine receptor is a fusion protein, comprising an extracellular binding domain, a hinge domain, a transmembrane domain, and a signaling domain. In some embodiments, the components of the receptor (that is, the domains of the receptor) are natural or synthetic. In some embodiments, the domains are human derived domains.

“FKBP” as used herein, is a FK506 binding protein domain. FKBP refers to a family of proteins that have prolyl isomerase activity and are related to the cyclophilins in function, though not in amino acid sequence. FKBPs have been identified in many eukaryotes from yeast to humans and function as protein folding chaperones for proteins containing proline residues. Along with cyclophilin, FKBPs belong to the immunophilin family. The term FKBP comprises, for example, FKBP12 as well as, proteins encoded by the genes AIP; AIPL1; FKBP1A; FKBP1B; FKBP2; FKBP3; FKBP5; FKBP6; FKBP7; FKBP8; FKBP9; FKBP9L; FKBP10; FKBP11; FKBP14; FKBP15; FKBP52; and/or L00541473; comprising homologs thereof and functional protein fragments thereof.

“FRB” as used herein, as a FKBP rapamycin binding domain. FRB domains are polypeptide regions (protein “domains”) that are configured to form a tripartite complex with an FKBP protein and rapamycin or rapalog thereof. FRB domains are present in a number of naturally occurring proteins, comprising mTOR proteins (also referred to in the literature as FRAP, RAPT 1, or RAFT) from human and other species; yeast proteins comprising Tor1 and/or Tor2; and/or a Candida FRAP homolog. Both FKBP and FRB are major constituents in the mammalian target of rapamycin (mTOR) signaling.

A “naked FKBP rapamycin binding domain polypeptide” or a “naked FRB domain polypeptide” refers to a polypeptide comprising only the amino acids of an FRB domain or a protein wherein at or about 90%, 91%, 92%, 93%, 94%, 95%, 96%, 97%, 98%, 99%, or 100% of the amino acids of the protein are amino acids of an FRB domain. The FRB domain can be expressed as a 12 kDa soluble protein (Chen, J. et al. (1995). Proc. Natl. Acad. Sci. U.S.A., 92(11):4947-4951). The FRB domain forms a four helix bundle, a common structural motif in globular proteins. Its overall dimensions are 30 Å by 45 Å by 30 Å, and all four helices) have short underhand connections similar to the cytochrome b562 fold (Choi, J. et al. (1996). Science, 273(5272):239-242). In some embodiments, the naked FRB domain comprises the amino acids of SEQ ID NO: 70 or SEQ ID NO: 71.

Cereblon interacts with damaged DNA binding protein 1 and forms an E3 ubiquitin ligase complex with Cullin 4 where it functions as a substrate receptor in which the proteins recognized by cereblon may be ubiquitinated and degraded by proteasomes. Proteasome-mediated degradation of unneeded or damaged proteins plays a very important role in maintaining regular function of a cell, such as cell survival, proliferation and/or growth. The binding of immunomodulatory imide drugs (IMIDs), e.g. thalidomide, to cereblon has been associated with teratogenicity and also the cytotoxicity of IMIDs, including lenalidomide. Cereblon is a key player in the binding, ubiquitination, and degradation of factors involved in maintaining function of myeloma cells.

“Cereblon thalidomide binding domain” refers to a binding domain that is an extracellular binding domain that interacts with an IMID, comprising, for example, thalidomide, pomalidomide, lenalidomide, apremilast, or related analogues. Some embodiments provided herein utilize cereblon thalidomide binding domain analogues or mutants thereof. In some embodiments, these extracellular binding domains are configured to simultaneously bind to an IMID ligand.

In some embodiments, the immunomodulatory imide drug used in the approaches described herein may comprise: thalidomide (including analogues, derivatives, and/or including pharmaceutically acceptable salts thereof. Thalidomide may include Immunoprin, Thalomid, Talidex, Talizer, Neurosedyn, α-(N-Phthalimido)glutarimide, 2-(2,6-dioxopiperidin-3-yl)-2,3-dihydro-1H-isoindole-1,3-dione); or pomalidomide (including analogues, derivatives, and/or including pharmaceutically acceptable salts thereof. Pomalidomide may include Pomalyst, Imnovid, (RS)-4-Amino-2-(2,6-dioxopiperidin-3-yl)isoindole-1,3-dione); or lenalidomide (including analogues, derivatives, and/or including pharmaceutically acceptable salts thereof. Lenalidomide may include Revlimid, (RS)-3-(4-Amino-1-oxo-1,3-dihydro-2H-isoindol-2-yl)piperidine-2,6-dione); or apremilast (including analogues, derivatives, and/or including pharmaceutically acceptable salts thereof. Apremilast may include Otezla, CC-10004, N-{2-[(1 S)-1-(3-Ethoxy-4-methoxyphenyl)-2-(methylsulfonyl) ethyl]-1,3-dioxo-2,3-dihydro-1H-isoindol-4-yl}acetamide); or any combinations thereof.

As used herein, the term “extracellular binding domain” refers to a domain of a complex that is outside of the cell, and which is configured to bind to a specific atom or molecule. In some embodiments, the extracellular binding domain of a CISC is a FKBP domain or a portion thereof. In some embodiments, the extracellular binding domain is an FRB domain or a portion thereof. In some embodiments, the extracellular binding domain is configured to bind a ligand or agent, thereby stimulating dimerization of two CISC components. In some embodiments, the extracellular binding domain is configured to bind to a cytokine receptor modulator.

As used herein, the term “cytokine receptor modulator” refers to an agent, which modulates the phosphorylation of a downstream target of a cytokine receptor, the activation of a signal transduction pathway associated with a cytokine receptor, and/or the expression of a particular protein such as a cytokine. Such an agent may directly or indirectly modulate the phosphorylation of a downstream target of a cytokine receptor, the activation of a signal transduction pathway associated with a cytokine receptor, and/or the expression of a particular protein such as a cytokine. Thus, examples of cytokine receptor modulators include, but are not limited to, cytokines, fragments of cytokines, fusion proteins and/or antibodies or binding portions thereof that immunospecifically bind to a cytokine receptor or a fragment thereof. Further, examples of cytokine receptor modulators include, but are not limited to, peptides, polypeptides (e.g., soluble cytokine receptors), fusion proteins and/or antibodies or binding portions thereof that immunospecifically bind to a cytokine or a fragment thereof.

As used herein, the term “activate” refers to an increase in at least one biological activity of a protein of interest. Similarly, the term “activation” refers to a state of a protein of interest being in a state of increased activity. The term “activatable” refers to the ability of a protein of interest to become activated in the presence of a signal, an agent, a ligand, a compound, or a stimulus. In some embodiments, a dimer, as described herein, is activated in the presence of a signal, an agent, a ligand, a compound, or a stimulus, and becomes a signaling competent dimer. As used herein, the term “signaling competent” refers to the ability or configuration of the dimer so as to be capable of initiating or sustaining a downstream signaling pathway.

As used herein, the term “hinge domain” refers to a domain that links the extracellular binding domain to the transmembrane domain, and may confer flexibility to the extracellular binding domain. In some embodiments, the hinge domain positions the extracellular domain close to the plasma membrane to minimize the potential for recognition by antibodies or binding fragments thereof. In some embodiments, the extracellular binding domain is located N-terminal to the hinge domain. In some embodiments, the hinge domain may be natural or synthetic.

As used herein, the term “transmembrane domain” or “TM domain” refers to a domain that is stable in a membrane, such as in a cell membrane. The terms “transmembrane span,” “integral protein,” and “integral domain” are also used herein. In some embodiments, the hinge domain and the extracellular domain is located N-terminal to the transmembrane domain. In some embodiments, the transmembrane domain is a natural or a synthetic domain. In some embodiments, the transmembrane domain is an IL-2 transmembrane domain.

As used herein, the term “signaling domain” refers to a domain of the fusion protein or CISC component that is involved in a signaling cascade inside the cell, such as a mammalian cell. A signaling domain refers to a signaling moiety that provides to cells, such as T cells, a signal which, in addition to the primary signal provided by for instance the CD3 zeta chain of the TCR/CD3 complex, mediates a cellular response, such as a T cell response, comprising, but not limited to, activation, proliferation, differentiation, and/or cytokine secretion. In some embodiments, the signaling domain is N-terminal to the transmembrane domain, the hinge domain, and the extracellular domain. In some embodiments, the signaling domain is a synthetic or a natural domain. In some embodiments, the signaling domain is a concatenated cytoplasmic signaling domain. In some embodiments, the signaling domain is a cytokine signaling domain. In some embodiments, the signaling domain is an antigen signaling domain. In some embodiments, the signaling domain is an interleukin-2 receptor subunit gamma (IL2Rγ or IL2Rg) domain. In some embodiments, the signaling domain is an interleukin-2 receptor subunit beta (IL2Rβ or IL2Rb) domain. In some embodiments, binding of an agent or ligand to the extracellular binding domain causes a signal transduction through the signaling domain by the activation of a signaling pathway, as a result of dimerization of the CISC components. As used herein, the term “signal transduction” refers to the activation of a signaling pathway by a ligand or an agent binding to the extracellular domain. Activation of a signal is a result of the binding of the extracellular domain to the ligand or agent, resulting in CISC dimerization.

As used herein, the term “IL2Rb” or “IL2Rβ” refers to an interleukin-2 receptor subunit beta. Similarly, the term “IL2Rg” or IL2Rγ” refers to an interleukin-2 receptor subunit gamma, and the term “IL2Ra” or “IL2Rα” refers to an interleukin-2 receptor subunit alpha. The IL-2 receptor has three forms, or chains, alpha, beta, and gamma, which are also subunits for receptors for other cytokines. IL2Rβ and IL2Rγ are members of the type I cytokine receptor family. “IL2R” as used herein refers to interleukin-2 receptor, which is involved in T cell-mediated immune responses. IL2R is involved in receptor-mediated endocytosis and transduction of mitogenic signals from interleukin 2. Similarly, the term “IL-2/15R” refers to a receptor signaling subunit that is shared by IL-2 and IL-15, and may include a subunit alpha (IL2/15Ra or IL2/15Rα), beta (IL2/15Rb or IL2/15Rβ, or gamma (IL2/15Rg or IL2/15Rγ).

In some embodiments, a chemical-induced signaling complex is a heterodimerization activated signaling complex comprising two components. In some embodiments, the first component comprises an extracellular binding domain that is one part of a heterodimerization pair, an optional hinge domain, a transmembrane domain, and one or more concatenated cytoplasmic signaling domains. In some embodiments, the second component comprises an extracellular binding domain that is the other part of a heterodimizeration pair, an optional hinge domain, a transmembrane domain, and one or more concatenated cytoplasmic signaling domains. Thus, in some embodiments, there are two distinct modification events. In some embodiments, the two CISC components are expressed in a cell, such as a mammalian cell. In some embodiments, the cell, such as a mammalian cell, or a population of cells, such as a population of mammalian cells, is contacted with a ligand or agent that causes heterodimerization, thereby initiating a signal. In some embodiments, a homodimerization pair dimerize, whereby a single CISC component is expressed in a cell, such as a mammalian cell, and the CISC components homodimerize to initiate a signal.

As used herein, the term “ligand” or “agent” refers to a molecule that has a desired biological effect. In some embodiments, a ligand is recognized by and bound by an extracellular binding domain, forming a tripartite complex comprising the ligand and two binding CISC components. Ligands include, but are not limited to, proteinaceous molecules, comprising, but not limited to, peptides, polypeptides, proteins, post-translationally modified proteins, antibodies, binding portions thereof; small molecules (less than 1000 Daltons), inorganic or organic compounds; and nucleic acid molecules comprising, but not limited to, double-stranded or single-stranded DNA, or double-stranded or single-stranded RNA (e.g., antisense, RNAi, etc.), aptamers, as well as, triple helix nucleic acid molecules. Ligands can be derived or obtained from any known organism (comprising, but not limited to, animals (e.g., mammals (human and non-human mammals)), plants, bacteria, fungi, and/or protista, or viruses) or from a library of synthetic molecules. In some embodiments, the ligand is a protein, an antibody or portion thereof, a small molecule, or a drug. In some embodiments, the ligand is rapamycin or a rapamycin analog (rapalogs). In some embodiments, the rapalog comprises variants of rapamycin having one or more of the following modifications relative to rapamycin: demethylation, elimination or replacement of the methoxy at C7, C42 and/or C29; elimination, derivatization or replacement of the hydroxy at C13, C43 and/or C28; reduction, elimination or derivatization of the ketone at C14, C24 and/or C30; replacement of the 6-membered pipecolate ring with a 5-membered prolyl ring; and embodiment substitution on the cyclohexyl ring or replacement of the cyclohexyl ring with a substituted cyclopentyl ring. Thus, in some embodiments, the rapalog is everolimus, merilimus, novolimus, pimecrolimus, ridaforolimus, tacrolimus, temsirolimus, umirolimus, zotarolimus, CCI-779, C20-methallylrapamycin, C16-(S)-3-methylindolerapamycin, C16-iRap, AP21967, sodium mycophenolic acid, benidipine hydrochloride, AP23573, or AP1903, or metabolites, derivatives, and/or combinations thereof. In some embodiments, the ligand is an IMID-class drug (e.g. thalidomide, pomalidomide, lenalidomide or related analogues).

As used herein, the term “simultaneous binding” refers to the binding of the ligand by two or more CISC components at the same time or, in some cases, at substantially the same time, to form a multicomponent complex, comprising the CISC components and the ligand component, and resulting in subsequent signal activation. Simultaneous binding requires that the CISC components are configured spatially to bind a single ligand, and also that both CISC components are configured to bind to the same ligand, including to different moieties on the same ligand.

As used herein, the term “selective expansion” refers to an ability of a desired cell, such as a mammalian cell, or a desired population of cells, such as a population of mammalian cells, to expand. In some embodiments, selective expansion refers to the generation or expansion of a pure population of cells, such as mammalian cells, that have undergone two genetic modification events. One component of a dimerization CISC is part of one modification and the other component is the other modification. Thus, one component of the heterodimerizing CISC is associated with each genetic modification. Exposure of the cells to a ligand allows for selective expansion of only the cells, such as mammalian cells, having both desired modifications. Thus, in some embodiments, the only cells, such as mammalian cells, that will be able to respond to contact with a ligand are those that express both components of the heterodimerization CISC.

Accordingly, in some embodiments, the ligand or agent used in the approaches described herein for chemical induction of the signaling complex may comprise: rapamycin (including analogues, derivatives, and including pharmaceutically acceptable salts thereof. Rapamycin may include Sirolimus, Rapamune, (3 S,6R,7E,9R,10R,12R,14S,15E,17E,19E,21S,23 S,26R,27R,34aS)-9,10,12,13,14,21,22,23,24,25,26,27,32,33,34,34a-hexadecahydro-9,27-dihydroxy-3-[(1R)-2-[(1 S,3R,4R)-4-hydroxy-3-methoxycyclohexyl]-1-methylethyl]-10,21-dimethoxy-6,8,12,14,20,26-hexamethyl-23,27-epoxy-3H-pyrido[2,1-c][1,4] oxaazacyclohentriacontine-1,5,11,28,29 (4H,6H,31H)-pentone); or everolimus (including analogues, derivatives, and including pharmaceutically acceptable salts thereof. Everolimus may include RAD001, Zortress, Certican, Afinitor, Votubia, 42-O-(2-hydroxyethyl)rapamycin, (1R,9S,12S,15R,16E,18R,19R,21R,23S,24E,26E,28E,30S,32S,35R)-1,18-dihydroxy-12-[(2R)-1-[(1 S,3R,4R)-4-(2-hydroxyethoxy)-3-methoxycyclohexyl]propan-2-yl]-19,30-dimethoxy-15,17,21,23,29,35-hexamethyl-11,36-di oxa-4-azatricyclo[30.3.1.0^{4,9}]hexatriaconta-16,24,26,28-tetraene-2,3,10,14,20-pentone); or merilimus (including analogues, derivatives, and including pharmaceutically acceptable salts thereof. Merilimus may include SAR943, 42-O-(tetrahydrofuran-3-yl)rapamycin (Merilimus-1); 42-O-(oxetan-3-yl)rapamycin (Merilimus-2), 42-O-(tetrahydropyran-3-yl)rapamycin (Merilimus-3), 42-O-(4-methyl, tetrahydrofuran-3-yl)rapamycin, 42-O-(2,5,5-trimethyl, tetrahydrofuran-3-yl) rapamycin, 42-O-(2,5-diethyl-2-methyl, tetrahydrofuran-3-yl)rapamycin, 42-O-(2H-Pyran-3-yl, tetrahydro-6-methoxy-2-methyl)rapamycin, or 42-O-(2H-Pyran-3-yl, tetrahydro-2,2-dimethyl-6-phenyl)rapamycin); novolimus (including analogues, derivatives, and including pharmaceutically acceptable salts thereof. Novolimus may include 16-O-Demethyl Rapamycin); or pimecrolimus (including analogues, derivatives, and including pharmaceutically acceptable salts thereof. Pimecrolimus may include Elidel, (3S,4R,5S,8R,9E,12S,14S,15R,16S,18R,19R,26aS)-3-((E)-2-((1R,3R,4S)-4-chloro-3 methoxycyclohexyl)-1-methylvinyl)-8-ethyl 5,6,8,11,12,13,14,15,16,17,18,19,24,26,26ahexadecahydro-5,19-epoxy-3H-pyrido(2,1-c)(1,4)oxaazacyclotricosine-1,17,20,21 (4H,23H)-tetrone 33-epi-Chloro-33-desoxyascomycin); or ridaforolimus (including analogues, derivatives, and including pharmaceutically acceptable salts thereof. Ridaforolimus may include AP23573, MK-8669, deforolimus, (1R,9S,12S,15R,16E,18R,19R,21R,23S,24E,26E,28E,30S,32S,35R)-12-((1R)-2-((1S,3R,4R)-4-((Dimethylphosphinoyl)oxy)-3-methoxycyclohexyl)-1-methylethyl)-1,18-dihydroxy-19,30-dimethoxy15,17,21,23,29,35-hexamethyl-11,36-dioxa-4-azatricyclo(30.3.1.04,9)hexatriaconta-16,24,26,28-tetraene-2,3,10,14,20-pentone); or tacrolimus (including analogues, derivatives, and including pharmaceutically acceptable salts thereof. Tacrolimus may include FK-506, fujimycin, Prograf, Advagraf, protopic, 3S-[3R*[E(1S*,3S*,4S*)],4S*,5R*,8S*,9E,12R*,14R*,15S*,16R*,18S*,19S*,26aR*5,6,8,11,12,13,14,15,16,17,18,19,24,25,26,26a- hexadecahydro-5,19-dihydroxy-3-[2-(4-hydroxy-3-methoxycyclohexyl)-1-methylethenyl]-14,16-dimethoxy-4,10,12,18-tetramethyl-8-(2-propenyl)-15,19-epoxy-3H-pyrido[2,1-c] [1,4] oxaazacyclotricosine-1,7,20,21(4H,23H)-tetrone, monohydrate); or temsirolimus (including analogues, derivatives, and including pharmaceutically acceptable salts thereof. Temsirolimus may include CCI-779, CCL-779, Torisel, (1R,2R,4S)-4-{(2R)-2-[(3S,6R,7E,9R,10R,12R,14S,15E,17E,19E,21S,23 S,26R,27R,34aS)-9,27-dihydroxy-10,21-dimethoxy-6,8,12,14,20,26-hexamethyl-1,5,11,28,29-pentaoxo-1,4,5,6,9,10,11,12,13,14,21,22,23,24,25,26,27,28,29,31,32,33,34,34a-tetracosahydro-3H-23,27-epoxypyrido[2,1-c][1,4]oxazacyclohentriacontin-3-yl]propyl}-2-methoxycyclohexyl 3-hydroxy-2-(hydroxymethyl)-2-methylpropanoate); or umirolimus (including analogues, derivatives, and including pharmaceutically acceptable salts thereof. Umirolimus may include Biolimus, Biolimus A9, BA9, TRM-986, 42-O-(2-ethoxyethyl) Rapamycin); or zotarolimus (including analogues, derivatives, and including pharmaceutically acceptable salts thereof. Zotarolimus may include ABT-578, (42S)-42-Deoxy-42-(1H-tetrazol-1-yl)-rapamycin); C20-methallylrapamycin (including analogues, derivatives, and including pharmaceutically acceptable salts thereof. C20-methallylrapamycin may include C20-Marap); or C16-(S)-3-methylindolerapamycin (including analogues, derivatives, and including pharmaceutically acceptable salts thereof. C16-(S)-3-methylindolerapamycin may include C16-iRap); or AP21967 (including analogues, derivatives, and including pharmaceutically acceptable salts thereof. AP21967 may include C-16-(S)-7-methylindolerapamycin); or sodium mycophenolic acid (including analogues, derivatives, and including pharmaceutically acceptable salts thereof. Sodium mycophenolic acid may include CellCept, Myfortic, (4E)-6-(4-Hydroxy-6-methoxy-7-methyl-3-oxo-1,3-dihydro-2-benzofuran-5-yl)-4-methylhex-4-enoic acid); or benidipine hydrochloride (including analogues, derivatives, and including pharmaceutically acceptable salts thereof. Benidipine hydrochloride may include Benidipinum, Coniel); or AP1903 (including analogues, derivatives, and including pharmaceutically acceptable salts thereof. AP1903 may include Rimiducid, [(1R)-3-(3,4-dimethoxyphenyl)-1-[3-[2-[2-[[2-[3-[(1R)-3-(3,4-dimethoxyphenyl)-1-[(2S)-1-[(2S)-2-(3,4,5-trimethoxyphenyl)butanoyl]piperidine-2 carbonyl]oxypropyl]phenoxy]acetyl]amino]ethylamino]-2-oxoethoxy]phenyl]propyl] (2S)-1-[(2S)-2-(3,4,5-trimethoxyphenyl)butanoyl]piperidine-2-carboxylate); or any combinations thereof.

As used herein, the term “gibberellin” refers to a synthetic or naturally occurring form of the diterpenoid acids that are synthesized by the terpenoid pathway in plastids and then modified in the endoplasmic reticulum and cytosol until they reach their biologically-active form. Gibberellin may be a natural gibberellin or an analogue thereof, including, for example, gibberellins derived from the ent-gibberellane skeleton, or synthesized via ent-kauren, including gibberellin 1 (GA1), GA2, GA3 . . . GA136, and analogues and derivatives thereof. In some embodiments, gibberellin or an analogue or derivative thereof is utilized for CISC dimerization.

As used herein, “SLF-TMP” or “synthetic ligand of FKBP linked to trimethoprim” refers to a dimerizer for CISC dimerization. In some embodiments, the SLF moiety binds to a first CISC component and the TMP moiety binds to a second CISC component, causing CISC dimerization. In some embodiments, SLF can bind, for example, to FKBP and TMP can bind to E. coli dihydrofolate reductase (eDHFR).

As used herein, the term “simultaneous binding” refers to the binding of the ligand by two or more CISC components at the same time or, in some cases, at substantially the same time, to form a multicomponent complex, comprising the CISC components and the ligand component, and resulting in subsequent signal activation. Simultaneous binding requires that the CISC components are configured spatially to bind a single ligand, and also that both CISC components are configured to bind to the same ligand, including to different moieties on the same ligand.

As used herein, the term “selective expansion” refers to an ability of a desired cell, such as a mammalian cell, or a desired population of cells, such as a population of mammalian cells, to expand. In some embodiments, selective expansion refers to the generation or expansion of a pure population of cells, such as mammalian cells, that have undergone two genetic modification events. One component of a dimerization CISC is part of one modification and the other component is the other modification. Thus, one component of the heterodimerizing CISC is associated with each genetic modification. Exposure of the cells to a ligand allows for selective expansion of only the cells, such as mammalian cells, having both desired modifications. Thus, in some embodiments, the only cells, such as mammalian cells, that will be able to respond to contact with a ligand are those that express both components of the heterodimerization CISC.

As used herein, “host cell” comprises any cell type, such as a mammalian cell, that is susceptible to transformation, transfection, or transduction, with a nucleic acid construct or vector. In some embodiments, the host cell, such as a mammalian cell, is a T cell or a T regulatory cell (T_{reg}). In some embodiments, the host cell, such as a mammalian cell, is a hematopoietic stem cell. In some embodiments, the host cell is a CD34+, CD8+, or a CD4+ cell. In some embodiments, the host cell is a CD8+T cytotoxic lymphocyte cell selected from the group consisting of naïve CD8+ T cells, central memory CD8+ T cells, effector memory CD8+ T cells, and bulk CD8+ T cells. In some embodiments, the host cell is a CD4+T helper lymphocyte cell selected from the group consisting of naïve CD4+ T cells, central memory CD4+ T cells, effector memory CD4+ T cells, and bulk CD4+ T cells. As used herein, the term “population of cells” refers to a group of cells, such as mammalian cells, comprising more than one cell. In some embodiments, a cell, such as a mammalian cell, is manufactured, wherein the cell comprises the protein sequence as described herein or an expression vector that encodes the protein sequence as described herein.

As used herein, the term “transformed” or “transfected” refers to a cell, such as a mammalian cell, tissue, organ, or organism into which a foreign polynucleotide molecule, such as a construct, has been introduced. The introduced polynucleotide molecule may be integrated into the genomic DNA of the recipient cell, such as a mammalian cell, tissue, organ, or organism such that the introduced polynucleotide molecule is inherited by subsequent progeny. A “transgenic” or “transfected” cell, such as a mammalian cell, or organism also comprises progeny of the cell or organism and progeny produced from a breeding program employing such a transgenic organism as a parent in a cross and exhibiting an altered phenotype resulting from the presence of a foreign polynucleotide molecule. The term “transgenic” refers to a bacteria, fungi, or plant containing one or more heterologous polynucleic acid molecules. “Transduction” refers to virus-mediated gene transfer into cells, such as mammalian cells.

As used herein, a “subject” refers to an animal that is the object of treatment, observation or experiment. “Animal” comprises cold- and warm-blooded vertebrates and invertebrates such as fish, shellfish, reptiles and, in particular, mammals. “Mammal” comprises, without limitation, mice, rats, rabbits, guinea pigs, dogs, cats, sheep, goats, cows, horses, primates, such as monkeys, chimpanzees, and apes, and, in particular, humans. In some embodiment, the subject is human.

In some embodiments, an effective amount of a ligand used for inducing dimerization is an amount of 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 20, 25, 30, 35, 40, 45, 50, 55, 60, 65, 70, 75, 80, 85, 90, 95, or 100 nM or a concentration within a range defined by any two of the aforementioned values.

A “marker sequence,” as described herein, encodes a protein that is used for selecting or tracking a protein or cell, such as a mammalian cell, that has a protein of interest. In the embodiments described herein, the fusion protein provided can comprise a marker sequence that can be selected in experiments, such as flow cytometry.

“Cytotoxic T lymphocyte” (CTL), as used herein, refers to a T lymphocyte that expresses CD8 on the surface thereof (e.g., a CD8⁺ T cell). In some embodiments, such cells are preferably “memory” T cells (T_{M} cells) that are antigen-experienced. In some embodiments, a cell for fusion protein secretion is provided. In some embodiments, the cell is a cytotoxic T lymphocyte. “Central memory” T cell (or “T_{CM}”) as used herein, refers to an antigen experienced CTL that expresses CD62L, CCR-7 and/or CD45R0 on the surface thereof, and does not express or has decreased expression of CD45RA, as compared to naive cells. In some embodiments, a cell for fusion protein secretion is provided. In some embodiments, the cell is a central memory T cell (T_{CM}). In some embodiments, the central memory cells are positive for expression of CD62L, CCR7, CD28, CD127, CD45RO, and/or CD95, and may have decreased expression of CD54RA, as compared to naïve cells. “Effector memory” T cell (or “T_{EM}”) as used herein refers to an antigen experienced T cell that does not express or has decreased expression of CD62L on the surface thereof, as compared to central memory cells, and does not express or has a decreased expression of CD45RA, as compared to naïve cell. In some embodiments, a cell for fusion protein secretion is provided. In some embodiments, the cell is an effector memory T cell. In some embodiments, effector memory cells are negative for expression of CD62L and/or CCR7, as compared to naïve cells or central memory cells, and may have variable expression of CD28 and/or CD45RA.

“Naïve T cells” as used herein, refers to a non-antigen experienced T lymphocyte that expresses CD62L and/or CD45RA, and does not express CD45RO−, as compared to central or effector memory cells. In some embodiments, a cell, such as a mammalian cell, for fusion protein secretion is provided. In some embodiments, the cell, such as a mammalian cell, is a naïve T cell. In some embodiments, naïve CD8+T lymphocytes are characterized by the expression of phenotypic markers of naïve T cells comprising CD62L, CCR7, CD28, CD127, and/or CD45RA.

“Effector” T cells as used herein, refers to antigen experienced cytotoxic T lymphocyte cells that do not express or have decreased expression of CD62L, CCR7, and/or CD28, and are positive for granzyme B and/or perforin, as compared to central memory or naïve T cells. In some embodiments, a cell, such as a mammalian cell, for fusion protein secretion is provided. In some embodiments, the cell, such as a mammalian cell, is an effector T cell. In some embodiments, the cell, such as a mammalian cell, does not express or have decreased expression of CD62L, CCR7, and/or CD28, and are positive for granzyme B and/or perforin, as compared to central memory or naïve T cells.

“Epitope” as used herein, refers to a part of an antigen or molecule that is recognized by the immune system comprising antibodies, T cells, and/or B-cells. Epitopes usually have at least 7 amino acids and can be a linear or a conformational epitope. In some embodiments, a cell, such as a mammalian cell, expressing a fusion protein is provided, wherein the cell further comprises a chimeric antigen receptor. In some embodiments, the chimeric antigen receptor comprises a scFv that can recognize an epitope on a cancer cell. “Isolating,” or “purifying” when used to describe the various polypeptides or nucleic acids disclosed herein, refers to a polypeptide or nucleic acid that has been identified and separated and/or recovered from a component of its natural environment. Preferably, the isolated polypeptide or nucleic acid is free of association with all components with which it is naturally associated. Contaminant components of its natural environment are materials that would typically interfere with diagnostic or therapeutic uses for the polypeptide or nucleic acid, and can include enzymes, hormones, and other proteinaceous or non-proteinaceous solutes. In some embodiments, a method is provided wherein the method comprises delivering the nucleic acid of any one of the embodiments described herein or the expression vector of any one of the embodiments described herein to a bacterial cell, mammalian cell or insect cell, growing the cell up in a culture, inducing expression of the fusion protein and purifying the fusion protein for treatment.

“Percent (%) amino acid sequence identity” with respect to the sequences identified herein, e.g., a CISC sequence, is defined as the percentage of amino acid residues in a candidate sequence that are identical with the amino acid residues in the reference sequence for each of the extracellular binding domain, hinge domain, transmembrane domain, and/or the signaling domain, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, comprising any algorithms needed to achieve maximal alignment over the full-length of the sequences being compared. For example, % amino acid sequence identity values generated using the WU-BLAST-2 computer program (Altschul, S. F. et al. (1996). Methods Enzymol., 266:460-480) uses several search parameters, most of which are set to the default values. Those that are not set to default values (e.g., the adjustable parameters) are set with the following values: overlap span=1, overlap fraction=0.125, word threshold (T)=11 and scoring matrix=BLOSUM62. In some embodiments of the CISC, the CISC comprises an extracellular binding domain, a hinge domain, a transmembrane domain, and a signaling domain, wherein each domain comprises a natural, synthetic, or a mutated or truncated form of the native domain. In some embodiments, a mutated or truncated form of any given domain comprises an amino acid sequence with 100%, 95%, 90%, 85% sequence identity, or a percent sequence identity that is within a range defined by any two of the aforementioned percentages to a sequence set forth in a sequence provided herein.

“CISC variant polypeptide sequence” or “CISC variant amino acid sequence” as used herein refers to a protein sequence as defined below having at least 80%, 85%, 90%, 95%, 98% or 99% amino acid sequence identity (or a percentage amino acid sequence identity within a range defined by any two of the aforementioned percentages) with the protein sequences provided herein, or a specifically derived fragment thereof, such as protein sequence for an extracellular binding domain, a hinge domain, a transmembrane domain and/or a signaling domain. Ordinarily, a CISC variant polypeptide or fragment thereof will have at least 80% amino acid sequence identity, more preferably at least 81% amino acid sequence identity, more preferably at least 82% amino acid sequence identity, more preferably at least 83% amino acid sequence identity, more preferably at least 84% amino acid sequence identity, more preferably at least 85% amino acid sequence identity, more preferably at least 86% amino acid sequence identity, more preferably at least 87% amino acid sequence identity, more preferably at least 88% amino acid sequence identity, more preferably at least 89% amino acid sequence identity, more preferably at least 90% amino acid sequence identity, more preferably at least 91% amino acid sequence identity, more preferably at least 92% amino acid sequence identity, more preferably at least 93% amino acid sequence identity, more preferably at least 94% amino acid sequence identity, more preferably at least 95% amino acid sequence identity, more preferably at least 96% amino acid sequence identity, more preferably at least 97% amino acid sequence identity, more preferably at least 98% amino acid sequence identity and yet more preferably at least 99% amino acid sequence identity with the amino acid sequence or a derived fragment thereof. Variants do not encompass the native protein sequence.

“T cells” or “T lymphocytes” as used herein can be from any mammalian, preferably primate, species, comprising monkeys, dogs, and humans. In some embodiments, the T cells are allogeneic (from the same species but different donor) as the recipient subject; in some embodiments the T cells are autologous (the donor and the recipient are the same); in some embodiments the T cells arc syngeneic (the donor and the recipients are different but are identical twins).

As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “comprising at least.” When used in the context of a process, the term “comprising” means that the process comprises at least the recited steps, but may include additional steps. When used in the context of a compound, composition or device, the term “comprising” means that the compound, composition or device comprises at least the recited features or components, but may also include additional features or components.

### Genome Editing Systems

Provided herein are systems for genome editing in a cell, e.g., a lymphocytic cell, to modulate the expression, function, and/or activity of a FOXP3, such as by targeted integration of a nucleic acid encoding a FOXP3 or a functional derivative thereof into the genome of the cell. The disclosures also provide, inter alia, systems for treating a subject having or suspected of having a disorder or health condition associated with FOXP3, employing ex vivo and/or in vivo genome editing. In some embodiments, the subject has or is suspected of having an autoimmune disease (e.g., IPEX syndrome) or a disorder that results from organ transplant (e.g., Graft-versus Host Disease (GVHD)).

In some embodiments, provided herein is a system comprising (a) a DNA endonuclease or nucleic acid encoding the DNA endonuclease; (b) a gRNA (e.g., an sgRNA) or nucleic acid encoding the gRNA, wherein the gRNA is capable of targeting the DNA endonuclease to a FOXP3 locus or a non-FOXP3 locus (e.g., AAVS1 (such as adeno-associated virus integration site in the genome of a cell, and (c) a donor template comprising a FOXP3 coding sequence. In some embodiments, the DNA endonuclease is selected from the group consisting of a Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cpf1 endonuclease, or a functional derivative thereof. In some embodiments, the DNA endonuclease is a Cas endonuclease, such as a Cas9 endonuclease (e.g., a Cas9 endonuclease from Streptococcus pyogenes). In some embodiments, the gRNA comprises a spacer sequence complementary to a target sequence in a FOXP3 locus. In some embodiments, the gRNA comprises a spacer sequence complementary to a target sequence in exon 1 of a FOXP3 locus. In some embodiments, the gRNA comprises a spacer sequence complementary to a target sequence in exon 1 of a FOXP3 locus. In some embodiments, the gRNA comprises a spacer sequence from any one of SEQ ID NOs: 1-7 and 27-29, or a variant thereof having no more than 3 mismatches compared to any one of SEQ ID NOs: 1-7 and 27-29. In some embodiments, the gRNA comprises a spacer sequence from any one of SEQ ID NOs: 1-7 or a variant thereof having no more than 3 mismatches compared to any one of SEQ ID NOs: 1-7. In some embodiments, the gRNA comprises a spacer sequence from any one of SEQ ID NOs: 2 and 5, or a variant thereof having no more than 3 mismatches compared to any one of SEQ ID NOs: 2 and 5. In some embodiments, the gRNA comprises a spacer sequence complementary to a target sequence in a non-FOXP3 locus (e.g., AAVS1 or TRAC). In some embodiments, the gRNA comprises a spacer sequence from any one of SEQ ID NOs: 15-20 or a variant thereof having no more than 3 mismatches compared to any one of SEQ ID NOs: 15-20. In some embodiments, the gRNA comprises a spacer sequence from any one of SEQ ID NOs: 33 and 34 or a variant thereof having no more than 3 mismatches compared to any one of SEQ ID NOs: 33 and 34. In some embodiments, the FOXP3 coding sequence encodes FOXP3 or a functional derivative thereof. In some embodiments, the FOXP3 coding sequence is a FOXP3 cDNA. In some embodiments, the nucleic acid sequence encoding a FOXP3 or a functional derivative thereof has at least at or about 70% sequence identity, e.g., at least at or about 75%, 80%, 85%, 90%, 92%, 93%, 94%, 95%, 96%, 97%, 98%, 99% or greater sequence identity, to a sequence according to SEQ ID NO: 68 or 69. In some embodiments, the system comprises the Cas DNA endonuclease. In some embodiments, the system comprises nucleic acid encoding the Cas DNA endonuclease. In some embodiments, the system comprises the gRNA. In some embodiments, the gRNA is an sgRNA. In some embodiments, the system comprises nucleic acid encoding the gRNA. In some embodiments, the system further comprises one or more additional gRNAs or nucleic acid encoding the one or more additional gRNAs.

In some embodiments, according to any of the systems described herein, the gRNA comprises a spacer sequence from any one of SEQ ID NOs: 1-7, 15-20, 27-29, 33, and 34, or a variant thereof having no more than 3 mismatches compared to any one of SEQ ID NOs: 1-7, 15-20, 27-29, 33, and 34. In some embodiments, the gRNA comprises a spacer sequence from any one of SEQ ID NOs: 1-7 or a variant thereof having no more than 3 mismatches compared to any one of SEQ ID NOs: 1-7. In some embodiments, the gRNA comprises a spacer sequence from any one of SEQ ID NOs: 2, 3, and 5 or a variant thereof having no more than 3 mismatches compared to any one of SEQ ID NOs: 2, 3, and 5. In some embodiments, the gRNA comprises a spacer sequence from SEQ ID NO: 2 or a variant thereof having no more than 3 mismatches compared to SEQ ID NO: 2. In some embodiments, the gRNA comprises a spacer sequence from SEQ ID NO: 3 or a variant thereof having no more than 3 mismatches compared to SEQ ID NO: 3. In some embodiments, the gRNA comprises a spacer sequence from SEQ ID NO: 5 or a variant thereof having no more than 3 mismatches compared to SEQ ID NO: 5.

In some embodiments, according to any of the systems described herein, the Cas DNA endonuclease is a Cas9 endonuclease. In some embodiments, the Cas9 endonuclease is from Streptococcus pyogenes (spCas9). In some embodiments, the Cas9 is from Staphylococcus lugdunensis (SluCas9).

In some embodiments, according to any of the systems described herein, the nucleic acid sequence encoding a FOXP3 or a functional derivative thereof is codon-optimized for expression in a host cell. In some embodiments, the nucleic acid sequence encoding a a FOXP3 or a functional derivative thereof has at least at or about 70% sequence identity, e.g., at least at or about 75%, 80%, 85%, 90%, 92%, 93%, 94%, 95%, 96%, 97%, 98%, 99% or greater sequence identity, to a sequence according to SEQ ID NO: 68 or 69. In some embodiments, the nucleic acid sequence encoding the FOXP3 or a functional derivative thereof is codon-optimized for expression in a human cell.

In some embodiments, according to any of the systems described herein, the system comprises a nucleic acid encoding the DNA endonuclease. In some embodiments, the nucleic acid encoding the DNA endonuclease is codon-optimized for expression in a host cell. In some embodiments, the nucleic acid encoding the DNA endonuclease is codon-optimized for expression in a human cell. In some embodiments, the nucleic acid encoding the DNA endonuclease is DNA, such as a DNA plasmid. In some embodiments, the nucleic acid encoding the DNA endonuclease is RNA, such as mRNA.

In some embodiments, according to any of the systems described herein, the donor template comprises a donor cassette comprising the nucleic acid sequence encoding a FOXP3 or a functional derivative thereof, and a promoter configured to express the FOXP3 or functional derivative thereof. Exemplary promoters include the MND promoter, PGK promoter, and EF1 promoter. In some embodiments, the promoter has a sequence of any one of SEQ ID NOs: 113-115 or a variant having at least 85% identity to any one of SEQ ID NOs: 113-115. In some embodiments, the donor template is encoded in an Adeno Associated Virus (AAV) vector. In some embodiments, the AAV vector is an AAV6 vector.

In some embodiments, according to any of the systems described herein, the donor template comprises a donor cassette comprising the nucleic acid sequence encoding a FOXP3 or a functional derivative thereof, and the donor template is configured such that the donor cassette is capable of being integrated into a genomic locus targeted by a gRNA in the system by homology directed repair (HDR). In some embodiments, the donor cassette is flanked on both sides by homology arms corresponding to sequences in the targeted genomic locus. In some embodiments, the homology arms are at least at or about 0.2 kb (such as at least at or about any of 0.3 kb, 0.4 kb, 0.5 kb, 0.6 kb, 0.7 kb, 0.8 kb, 0.9 kb, 1 kb, or greater) in length. In some embodiments, the homology arms are at least at or about 0.4 kb, e.g., 0.45 kb, 0.6 kb, or 0.8 kb, in length. Exemplary homology arms include 5′-homology arms having the sequence of any one of SEQ ID NOs: 90-97 and 106-107, and 3′-homology arms having the sequence of any one of SEQ ID NOs: 98-105 and 108-109. Exemplary homology arms further include homology arms from a donor template having the sequence of SEQ ID NO: 37 or 38. Exemplary donor templates include donor templates having the sequence of SEQ ID NO: 37 or 38. In some embodiments, the donor template is encoded in an Adeno Associated Virus (AAV) vector. In some embodiments, the AAV vector is an AAV2, AAV5, or AAV6 vector. In some embodiments, the AAV vector is an AAV6 vector.

In some embodiments, according to any of the systems described herein, the donor template comprises a donor cassette comprising the nucleic acid sequence encoding a FOXP3 or a functional derivative thereof, and the donor template is configured such that the donor cassette is capable of being integrated into a genomic locus targeted by a gRNA in the system by non-homologous end joining (NHEJ). In some embodiments, the donor cassette is flanked on one or both sides by a gRNA target site. In some embodiments, the donor cassette is flanked on both sides by a gRNA target site. In some embodiments, the gRNA target site is a target site for a gRNA in the system. In some embodiments, the gRNA target site of the donor template is the reverse complement of a cell genome gRNA target site for a gRNA in the system. In some embodiments, the donor template is encoded in an Adeno Associated Virus (AAV) vector. In some embodiments, the AAV vector is an AAV2, AAV5, or AAV6 vector. In some embodiments, the AAV vector is an AAV6 vector.

In some embodiments, according to any of the systems described herein comprising a donor template comprising a donor cassette comprising the nucleic acid sequence encoding a FOXP3 or a functional derivative thereof, the donor cassette comprises a woodchuck hepatitis virus (WHIP) posttranscriptional regulatory element (WPRE). In some embodiments, the WPRE is a full-length WPRE. In some embodiments, the WPRE is a truncated WPRE. Exemplary WPREs include WPREs from a donor template having the sequence of any one of SEQ ID NOs: 135-147. Exemplary donor templates having a WPRE include donor templates having the sequence of any one of SEQ ID NOs: 135-147.

In some embodiments, according to any of the systems described herein comprising a donor template comprising a donor cassette comprising the nucleic acid sequence encoding a FOXP3 or a functional derivative thereof, the donor cassette comprises a ubiquitous chromatin opening element (UCOE). Exemplary UCOEs include UCOEs from a donor template having the sequence of any one of SEQ ID NOs: 158, 159, or 162. Exemplary donor templates having a UCOE include donor templates having the sequence of any one of SEQ ID NOs: 158, 159, or 162.

In some embodiments, according to any of the systems described herein comprising a donor template comprising a donor cassette comprising the nucleic acid sequence encoding a FOXP3 or a functional derivative thereof, the donor cassette comprises a low affinity nerve growth factor receptor (LNGFR) coding sequence. In some embodiments, the LNGFR coding sequence is upstream of the nucleic acid sequence encoding a FOXP3 or a functional derivative thereof. In some embodiments, the LNGFR coding sequence is downstream of the nucleic acid sequence encoding a FOXP3 or a functional derivative thereof. Exemplary LNGFR coding sequences include LNGFR coding sequences from a donor template having the sequence of any one of SEQ ID NOs: 37, 38, 40, 42, 46, 47, 74, 76, 80, and 81. Exemplary LNGFR coding sequences include the sequence of any one of SEQ ID NOs: 88 and 118, or a variant having at least 85% identity to any one of SEQ ID NOs: 88 and 118.

In some embodiments, according to any of the systems described herein comprising a donor template comprising a donor cassette comprising the nucleic acid sequence encoding a FOXP3 or a functional derivative thereof, the donor cassette comprises a 3′ untranslated region (UTR) linked to the 3′ end of the nucleic acid sequence encoding a FOXP3 or a functional derivative thereof. In some embodiments, the 3′ UTR comprises an SV40-polyA signal. Exemplary 3′UTRs comprising an SV40-polyA signal include the 3′UTR having the sequence of SEQ ID NO: 116. In some embodiments, the 3′ UTR comprises a 3′ UTR derived from a human FOXP3 gene. Exemplary 3′UTRs derived from a human FOXP3 gene include the 3′UTR having the sequence of SEQ ID NO: 117.

In some embodiments, according to any of the systems described herein, the donor template comprises a donor cassette comprising the nucleic acid sequence encoding a FOXP3 or a functional derivative thereof, and the donor template further comprises a nucleic acid encoding a 2A self-cleaving peptide between adjacent system component-encoding nucleic acids. In some embodiments, the donor template comprise nucleic acid encoding a 2A self-cleaving peptide between each of the adjacent system component-encoding nucleic acids. In some embodiments, each of the 2A self-cleaving peptides is, independently, a T2A self-cleaving peptide or a P2A self-cleaving peptide. For example, in some embodiments, the donor template comprises, in order from 5′ to 3′, a promoter, a nucleic acid encoding expression of a FOXP3 or functional variant thereof, nucleic acid encoding a 2A self-cleaving peptide, and a nucleic acid encoding a selectable marker. In some embodiments, the donor template comprises a nucleic acid of SEQ ID NO: 89, or a variant of a nucleic acid having at least 85% identity to SEQ ID NO: 89. In some embodiments, the donor template is encoded in an Adeno Associated Virus (AAV) vector. In some embodiments, the AAV vector is an AAV6 vector.

In some embodiments, according to any of the systems described herein, the DNA endonuclease or nucleic acid encoding the DNA endonuclease is formulated in a liposome or lipid nanoparticle. In some embodiments, the liposome or lipid nanoparticle also comprises the gRNA. In some embodiments, the liposome or lipid nanoparticle is a lipid nanoparticle. In some embodiments, the system comprises a lipid nanoparticle comprising nucleic acid encoding the DNA endonuclease and the gRNA. In some embodiments, the nucleic acid encoding the DNA endonuclease is an mRNA encoding the DNA endonuclease.

In some embodiments, according to any of the systems described herein, the DNA endonuclease is complexed with the gRNA, forming a ribonucleoprotein (RNP) complex.

### Nucleic Acids

Genome-Targeting Nucleic Acid or Guide RNA

The present disclosure provides a genome-targeting nucleic acid that can direct the activities of an associated polypeptide (e.g., a site-directed polypeptide or DNA endonuclease) to a specific target sequence within a target nucleic acid. In some embodiments, the genome-targeting nucleic acid is an RNA. A genome-targeting RNA is referred to as a “guide RNA” or “gRNA” herein. A guide RNA has at least a spacer sequence that can hybridize to a target nucleic acid sequence of interest and a CRISPR repeat sequence. In Type II systems, the gRNA also has a second RNA referred to as a tracrRNA sequence. In the Type II guide RNA (gRNA), the CRISPR repeat sequence and tracrRNA sequence hybridize to each other to form a duplex. In the Type V guide RNA (gRNA), the crRNA forms a duplex. In both systems, the duplex binds a site-directed polypeptide such that the guide RNA and site-direct polypeptide form a complex. The genome-targeting nucleic acid provides target specificity to the complex by virtue of its association with the site-directed polypeptide. The genome-targeting nucleic acid thus directs the activity of the site-directed polypeptide.

In some embodiments, the genome-targeting nucleic acid is a double-molecule guide RNA. In some embodiments, the genome-targeting nucleic acid is a single-molecule guide RNA. A double-molecule guide RNA has two strands of RNA. The first strand has in the 5′ to 3′ direction, an optional spacer extension sequence, a spacer sequence and a minimum CRISPR repeat sequence. The second strand has a minimum tracrRNA sequence (complementary to the minimum CRISPR repeat sequence), a 3′ tracrRNA sequence and an optional tracrRNA extension sequence. A single-molecule guide RNA (sgRNA) in a Type II system has, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence and an optional tracrRNA extension sequence. The optional tracrRNA extension may have elements that contribute additional functionality (e.g., stability) to the guide RNA. The single-molecule guide linker links the minimum CRISPR repeat and the minimum tracrRNA sequence to form a hairpin structure. The optional tracrRNA extension has one or more hairpins. A single-molecule guide RNA (sgRNA) in a Type V system has, in the 5′ to 3′ direction, a minimum CRISPR repeat sequence and a spacer sequence.

By way of illustration, guide RNAs used in the CRISPR/Cas/Cpf1 system, or other smaller RNAs can be readily synthesized by chemical means as illustrated below and described in the art. While chemical synthetic procedures are continually expanding, purifications of such RNAs by procedures such as high performance liquid chromatography (HPLC, which avoids the use of gels such as PAGE) tends to become more challenging as polynucleotide lengths increase significantly beyond a hundred or so nucleotides. One approach used for generating RNAs of greater length is to produce two or more molecules that are ligated together. Much longer RNAs, such as those encoding a Cas9 or Cpf1 endonuclease, are more readily generated enzymatically. Various types of RNA modifications can be introduced during or after chemical synthesis and/or enzymatic generation of RNAs, e.g., modifications that enhance stability, reduce the likelihood or degree of innate immune response, and/or enhance other attributes, as described in the art.

In some embodiments, provided herein is a guide RNA (gRNA) comprising a spacer sequence that is complementary to a genomic sequence within or near a FOXP3 locus in a cell. In some embodiments, the gRNA comprises a spacer sequence from any one of SEQ ID NOs: 1-7 and 27-29 or a variant thereof having no more than 3 mismatches compared to any one of SEQ ID NOs: 1-7 and 27-29. In some embodiments, the gRNA comprises a spacer sequence from any one of SEQ ID NOs: 1-7 or a variant thereof having no more than 3 mismatches compared to any one of SEQ ID NOs: 1-7. In some embodiments, the gRNA comprises a spacer sequence from any one of SEQ ID NOs: 2, 3, and 5 or a variant thereof having no more than 3 mismatches compared to any one of SEQ ID NOs: 2, 3, and 5.

In some embodiments, provided herein is a guide RNA (gRNA) comprising a spacer sequence that is complementary to a genomic sequence within or near an AAVS1 locus in a cell. In some embodiments, the gRNA comprises a spacer sequence from any one of SEQ ID NOs: 15-20 or a variant thereof having no more than 3 mismatches compared to any one of SEQ ID NOs: 15-20.

Guide RNA made by in vitro transcription may contain mixtures of full length and partial guide RNA molecules. Chemically synthesized guide RNA molecules are generally composed of >75% full length guide molecules and in addition may contain chemically modified bases, such as those that make the guide RNA more resistant to cleavage by nucleases in the cell.

Spacer Extension Sequence

In some embodiments of genome-targeting nucleic acids, a spacer extension sequence can modify activity, provide stability and/or provide a location for modifications of a genome-targeting nucleic acid. A spacer extension sequence can modify on- or off-target activity or specificity. In some embodiments, a spacer extension sequence is provided. A spacer extension sequence can have a length of more than 1, 5, 10, 15, 20, 25, 30, 35, 40, 45, 50, 60, 70, 80, 90, 100, 120, 140, 160, 180, 200, 220, 240, 260, 280, 300, 320, 340, 360, 380, 400, 1000, 2000, 3000, 4000, 5000, 6000, or 7000 or more nucleotides. A spacer extension sequence can have a length of at or about 1, 5, 10, 15, 20, 25, 30, 35, 40, 45, 50, 60, 70, 80, 90, 100, 120, 140, 160, 180, 200, 220, 240, 260, 280, 300, 320, 340, 360, 380, 400, 1000, 2000, 3000, 4000, 5000, 6000, or 7000 or more nucleotides. A spacer extension sequence can have a length of less than 1, 5, 10, 15, 20, 25, 30, 35, 40, 45, 50, 60, 70, 80, 90, 100, 120, 140, 160, 180, 200, 220, 240, 260, 280, 300, 320, 340, 360, 380, 400, 1000, 2000, 3000, 4000, 5000, 6000, 7000, or more nucleotides. In some embodiments, a spacer extension sequence is less than 10 nucleotides in length. In some embodiments, a spacer extension sequence is between 10-30 nucleotides in length. In some embodiments, a spacer extension sequence is between 30-70 nucleotides in length.

In some embodiments, the spacer extension sequence has another moiety (e.g., a stability control sequence, an endoribonuclease binding sequence, a ribozyme). In some embodiments, the moiety decreases or increases the stability of a nucleic acid targeting nucleic acid. In some embodiments, the moiety is a transcriptional terminator segment (such as a transcription termination sequence). In some embodiments, the moiety functions in a eukaryotic cell. In some embodiments, the moiety functions in a prokaryotic cell. In some embodiments, the moiety functions in both eukaryotic and prokaryotic cells. Non-limiting examples of suitable moieties include: a 5′ cap (e.g., a 7-methylguanylate cap (m7 G)), a riboswitch sequence (e.g., to allow for regulated stability and/or regulated accessibility by proteins and protein complexes), a sequence that forms a dsRNA duplex (such as a hairpin), a sequence that targets the RNA to a subcellular location (e.g., nucleus, mitochondria, chloroplasts, and the like), a modification or sequence that provides for tracking (e.g., direct conjugation to a fluorescent molecule, conjugation to a moiety that facilitates fluorescent detection, a sequence that allows for fluorescent detection, etc.), and/or a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like).

Spacer Sequence

The spacer sequence hybridizes to a sequence in a target nucleic acid of interest. The spacer of a genome-targeting nucleic acid interacts with a target nucleic acid in a sequence-specific manner via hybridization (such as base pairing). The nucleotide sequence of the spacer thus varies depending on the sequence of the target nucleic acid of interest.

In a CRISPR/Cas system herein, the spacer sequence is designed to hybridize to a target nucleic acid that is located 5′ of a PAM of the Cas9 enzyme used in the system. The spacer can perfectly match the target sequence or can have mismatches. Each Cas9 enzyme has a particular PAM sequence that it recognizes in a target DNA. For example, S. pyogenes recognizes in a target nucleic acid a PAM that has the sequence 5′-NRG-3′, where R has either A or G, where N is any nucleotide and N is immediately 3′ of the target nucleic acid sequence targeted by the spacer sequence.

In some embodiments, the target nucleic acid sequence has 20 nucleotides. In some embodiments, the target nucleic acid has less than 20 nucleotides. In some embodiments, the target nucleic acid has more than 20 nucleotides. In some embodiments, the target nucleic acid has at least: 5, 10, 15, 16, 17, 18, 19, 20, 21, 22, 23, 24, 25, 30, or more nucleotides. In some embodiments, the target nucleic acid has at most: 5, 10, 15, 16, 17, 18, 19, 20, 21, 22, 23, 24, 25, 30, or more nucleotides. In some embodiments, the target nucleic acid sequence has 20 bases immediately 5′ of the first nucleotide of the PAM. In some embodiments, the PAM sequence used in the compositions and methods of the present disclosure as a sequence recognized by S.p. Cas9 is NGG.

In some embodiments, the spacer sequence that hybridizes to the target nucleic acid has a length of at least at or about 6 nucleotides (nt). The spacer sequence can be at least at or about 6 nt, about 10 nt, about 15 nt, about 18 nt, about 19 nt, about 20 nt, about 25 nt, about 30 nt, about 35 nt or about 40 nt, from about 6 nt to about 80 nt, from about 6 nt to about 50 nt, from about 6 nt to about 45 nt, from about 6 nt to about 40 nt, from about 6 nt to about 35 nt, from about 6 nt to about 30 nt, from about 6 nt to about 25 nt, from about 6 nt to about 20 nt, from about 6 nt to about 19 nt, from about 10 nt to about 50 nt, from about 10 nt to about 45 nt, from about 10 nt to about 40 nt, from about 10 nt to about 35 nt, from about 10 nt to about 30 nt, from about 10 nt to about 25 nt, from about 10 nt to about 20 nt, from about 10 nt to about 19 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, or from about 20 nt to about 60 nt. In some embodiments, the spacer sequence has 20 nucleotides. In some embodiments, the spacer has 19 nucleotides. In some embodiments, the spacer has 18 nucleotides. In some embodiments, the spacer has 17 nucleotides. In some embodiments, the spacer has 16 nucleotides. In some embodiments, the spacer has 15 nucleotides.

In some embodiments, the percent complementarity between the spacer sequence and the target nucleic acid is at least about 30%, at least about 40%, at least about 50%, at least about 60%, at least about 65%, at least about 70%, at least about 75%, at least about 80%, at least about 85%, at least about 90%, at least about 95%, at least about 97%, at least about 98%, at least about 99%, or 100%. In some embodiments, the percent complementarity between the spacer sequence and the target nucleic acid is at most about 30%, at most about 40%, at most about 50%, at most about 60%, at most about 65%, at most about 70%, at most about 75%, at most about 80%, at most about 85%, at most about 90%, at most about 95%, at most about 97%, at most about 98%, at most about 99%, or 100%. In some embodiments, the percent complementarity between the spacer sequence and the target nucleic acid is 100% over the six contiguous 5′-most nucleotides of the target sequence of the complementary strand of the target nucleic acid. In some embodiments, the percent complementarity between the spacer sequence and the target nucleic acid is at least 60% over about 20 contiguous nucleotides. In some embodiments, the length of the spacer sequence and the target nucleic acid can differ by 1 to 6 nucleotides, which can be thought of as a bulge or bulges.

In some embodiments, the spacer sequence is designed or chosen using a computer program. The computer program can use variables, such as predicted melting temperature, secondary structure formation, predicted annealing temperature, sequence identity, genomic context, chromatin accessibility, % GC, frequency of genomic occurrence (e.g., of sequences that are identical or are similar but vary in one or more spots as a result of mismatch, insertion, or deletion), methylation status, presence of SNPs, and the like.

Minimum CRISPR Repeat Sequence

In some embodiments, a minimum CRISPR repeat sequence is a sequence with at least at or about 30%, about 40%, about 50%, about 60%, about 65%, about 70%, about 75%, about 80%, about 85%, about 90%, about 95%, or 100% sequence identity to a reference CRISPR repeat sequence (e.g., crRNA from S. pyogenes).

In some embodiments, a minimum CRISPR repeat sequence has nucleotides that can hybridize to a minimum tracrRNA sequence in a cell. The minimum CRISPR repeat sequence and a minimum tracrRNA sequence form a duplex, such as a base-paired double-stranded structure. Together, the minimum CRISPR repeat sequence and the minimum tracrRNA sequence bind to the site-directed polypeptide. At least a part of the minimum CRISPR repeat sequence hybridizes to the minimum tracrRNA sequence. In some embodiments, at least a part of the minimum CRISPR repeat sequence has at least about 30%, about 40%, about 50%, about 60%, about 65%, about 70%, about 75%, about 80%, about 85%, about 90%, about 95%, or 100% complementarity to the minimum tracrRNA sequence. In some embodiments, at least a part of the minimum CRISPR repeat sequence has at most about 30%, about 40%, about 50%, about 60%, about 65%, about 70%, about 75%, about 80%, about 85%, about 90%, about 95%, or 100% complementarity to the minimum tracrRNA sequence.

The minimum CRISPR repeat sequence can have a length from about 7 nucleotides to about 100 nucleotides. For example, the length of the minimum CRISPR repeat sequence is from at or about 7 nucleotides (nt) to about 50 nt, from about 7 nt to about 40 nt, from about 7 nt to about 30 nt, from about 7 nt to about 25 nt, from about 7 nt to about 20 nt, from about 7 nt to about 15 nt, from about 8 nt to about 40 nt, from about 8 nt to about 30 nt, from about 8 nt to about 25 nt, from about 8 nt to about 20 nt, from about 8 nt to about 15 nt, from about 15 nt to about 100 nt, from about 15 nt to about 80 nt, from about 15 nt to about 50 nt, from about 15 nt to about 40 nt, from about 15 nt to about 30 nt, or from about 15 nt to about 25 nt. In some embodiments, the minimum CRISPR repeat sequence is approximately 9 nucleotides in length. In some embodiments, the minimum CRISPR repeat sequence is approximately 12 nucleotides in length.

In some embodiments, the minimum CRISPR repeat sequence is at least about 60% identical to a reference minimum CRISPR repeat sequence (e.g., wild-type crRNA from S. pyogenes) over a stretch of at least 6, 7, or 8 contiguous nucleotides. For example, the minimum CRISPR repeat sequence is at least at or about 65% identical, at least about 70% identical, at least about 75% identical, at least about 80% identical, at least about 85% identical, at least about 90% identical, at least about 95% identical, at least about 98% identical, at least about 99% identical or 100% identical to a reference minimum CRISPR repeat sequence over a stretch of at least 6, 7, or 8 contiguous nucleotides.

Minimum tracrRNA Sequence

In some embodiments, a minimum tracrRNA sequence is a sequence with at least at or about 30%, about 40%, about 50%, about 60%, about 65%, about 70%, about 75%, about 80%, about 85%, about 90%, about 95%, or 100% sequence identity to a reference tracrRNA sequence (e.g., wild type tracrRNA from S. pyogenes).

In some embodiments, a minimum tracrRNA sequence has nucleotides that hybridize to a minimum CRISPR repeat sequence in a cell. A minimum tracrRNA sequence and a minimum CRISPR repeat sequence form a duplex, such as a base-paired double-stranded structure. Together, the minimum tracrRNA sequence and the minimum CRISPR repeat bind to a site-directed polypeptide. At least a part of the minimum tracrRNA sequence can hybridize to the minimum CRISPR repeat sequence. In some embodiments, the minimum tracrRNA sequence is at least about 30%, about 40%, about 50%, about 60%, about 65%, about 70%, about 75%, about 80%, about 85%, about 90%, about 95%, or 100% complementarity to the minimum CRISPR repeat sequence.

The minimum tracrRNA sequence can have a length from about 7 nucleotides to about 100 nucleotides. For example, the minimum tracrRNA sequence can be from about 7 nucleotides (nt) to about 50 nt, from about 7 nt to about 40 nt, from about 7 nt to about 30 nt, from about 7 nt to about 25 nt, from about 7 nt to about 20 nt, from about 7 nt to about 15 nt, from about 8 nt to about 40 nt, from about 8 nt to about 30 nt, from about 8 nt to about 25 nt, from about 8 nt to about 20 nt, from about 8 nt to about 15 nt, from about 15 nt to about 100 nt, from about 15 nt to about 80 nt, from about 15 nt to about 50 nt, from about 15 nt to about 40 nt, from about 15 nt to about 30 nt or from about 15 nt to about 25 nt long. In some embodiments, the minimum tracrRNA sequence is approximately 9 nucleotides in length. In some embodiments, the minimum tracrRNA sequence is approximately 12 nucleotides. In some embodiments, the minimum tracrRNA consists of tracrRNA nt 23-48 described in Jinek, M. et al. (2012). Science, 337(6096):816-821.

In some embodiments, the minimum tracrRNA sequence is at least about 60% identical to a reference minimum tracrRNA (e.g., wild type, tracrRNA from S. pyogenes) sequence over a stretch of at least 6, 7, or 8 contiguous nucleotides. For example, the minimum tracrRNA sequence is at least at or about 65% identical, about 70% identical, about 75% identical, about 80% identical, about 85% identical, about 90% identical, about 95% identical, about 98% identical, about 99% identical or 100% identical to a reference minimum tracrRNA sequence over a stretch of at least 6, 7, or 8 contiguous nucleotides.

In some embodiments, the duplex between the minimum CRISPR RNA and the minimum tracrRNA has a double helix. In some embodiments, the duplex between the minimum CRISPR RNA and the minimum tracrRNA has at least about 1, 2, 3, 4, 5, 6, 7, 8, 9, or 10 or more nucleotides. In some embodiments, the duplex between the minimum CRISPR RNA and the minimum tracrRNA has at most about 1, 2, 3, 4, 5, 6, 7, 8, 9, or 10 or more nucleotides.

In some embodiments, the duplex has a mismatch (such as the two strands of the duplex are not 100% complementary). In some embodiments, the duplex has at least about 1, 2, 3, 4, or 5 or mismatches. In some embodiments, the duplex has at most about 1, 2, 3, 4, or 5 or mismatches. In some embodiments, the duplex has no more than 2 mismatches.

Bulges

In some embodiments, there is a “bulge” in the duplex between the minimum CRISPR RNA and the minimum tracrRNA. The bulge is an unpaired region of nucleotides within the duplex. In some embodiments, the bulge contributes to the binding of the duplex to the site-directed polypeptide. A bulge has, on one side of the duplex, an unpaired 5′-XXXY-3′ where X is any purine and Y has a nucleotide that can form a wobble pair with a nucleotide on the opposite strand, and an unpaired nucleotide region on the other side of the duplex. The number of unpaired nucleotides on the two sides of the duplex can be different.

In one example, the bulge has an unpaired purine (e.g., adenine) on the minimum CRISPR repeat strand of the bulge. In some embodiments, a bulge has an unpaired 5′-AAGY-3′ of the minimum tracrRNA sequence strand of the bulge, where Y has a nucleotide that can form a wobble pairing with a nucleotide on the minimum CRISPR repeat strand.

In some embodiments, a bulge on the minimum CRISPR repeat side of the duplex has at least 1, 2, 3, 4, or 5 or more unpaired nucleotides. In some embodiments, a bulge on the minimum CRISPR repeat side of the duplex has at most 1, 2, 3, 4, or 5 or more unpaired nucleotides. In some embodiments, a bulge on the minimum CRISPR repeat side of the duplex has 1 unpaired nucleotide.

In some embodiments, a bulge on the minimum tracrRNA sequence side of the duplex has at least 1, 2, 3, 4, 5, 6, 7, 8, 9, or 10 or more unpaired nucleotides. In some embodiments, a bulge on the minimum tracrRNA sequence side of the duplex has at most 1, 2, 3, 4, 5, 6, 7, 8, 9, or 10 or more unpaired nucleotides. In some embodiments, a bulge on a second side of the duplex (e.g., the minimum tracrRNA sequence side of the duplex) has 4 unpaired nucleotides.

In some embodiments, a bulge has at least one wobble pairing. In some embodiments, a bulge has at most one wobble pairing. In some embodiments, a bulge has at least one purine nucleotide. In some embodiments, a bulge has at least 3 purine nucleotides. In some embodiments, a bulge sequence has at least 5 purine nucleotides. In some embodiments, a bulge sequence has at least one guanine nucleotide. In some embodiments, a bulge sequence has at least one adenine nucleotide.

Hairpins

In various embodiments, one or more hairpins are located 3′ to the minimum tracrRNA in the 3′ tracrRNA sequence.

In some embodiments, the hairpin starts at least about 1, 2, 3, 4, 5, 6, 7, 8, 9, 10, 15, or 20 or more nucleotides 3′ from the last paired nucleotide in the minimum CRISPR repeat and minimum tracrRNA sequence duplex. In some embodiments, the hairpin can start at most about 1, 2, 3, 4, 5, 6, 7, 8, 9, or 10 or more nucleotides 3′ of the last paired nucleotide in the minimum CRISPR repeat and minimum tracrRNA sequence duplex.

In some embodiments, a hairpin has at least at or about 1, 2, 3, 4, 5, 6, 7, 8, 9, 10, 15, or 20 or more consecutive nucleotides. In some embodiments, a hairpin has at most about 1, 2, 3, 4, 5, 6, 7, 8, 9, 10, 15, or more consecutive nucleotides.

In some embodiments, a hairpin has a CC di-nucleotide (such as two consecutive cytosine nucleotides).

In some embodiments, a hairpin has duplexed nucleotides (e.g., nucleotides in a hairpin, hybridized together). For example, a hairpin has a CC di-nucleotide that is hybridized to a GG di-nucleotide in a hairpin duplex of the 3′ tracrRNA sequence.

One or more of the hairpins can interact with guide RNA-interacting regions of a site-directed polypeptide.

In some embodiments there are two or more hairpins, and in some embodiments there are three or more hairpins.

3′ tracrRNA Sequence

In some embodiments, a 3′ tracrRNA sequence has a sequence with at least about 30%, about 40%, about 50%, about 60%, about 65%, about 70%, about 75%, about 80%, about 85%, about 90%, about 95%, or 100% sequence identity to a reference tracrRNA sequence (e.g., a tracrRNA from S. pyogenes).

In some embodiments, the 3′ tracrRNA sequence has a length from at or about 6 nucleotides to about 100 nucleotides. For example, the 3′ tracrRNA sequence can have a length from about 6 nucleotides (nt) to about 50 nt, from about 6 nt to about 40 nt, from about 6 nt to about 30 nt, from about 6 nt to about 25 nt, from about 6 nt to about 20 nt, from about 6 nt to about 15 nt, from about 8 nt to about 40 nt, from about 8 nt to about 30 nt, from about 8 nt to about 25 nt, from about 8 nt to about 20 nt, from about 8 nt to about 15 nt, from about 15 nt to about 100 nt, from about 15 nt to about 80 nt, from about 15 nt to about 50 nt, from about 15 nt to about 40 nt, from about 15 nt to about 30 nt, or from about 15 nt to about 25 nt. In some embodiments, the 3′ tracrRNA sequence has a length of approximately 14 nucleotides.

In some embodiments, the 3′ tracrRNA sequence is at least about 60% identical to a reference 3′ tracrRNA sequence (e.g., wild type 3′ tracrRNA sequence from S. pyogenes) over a stretch of at least 6, 7, or 8 contiguous nucleotides. For example, the 3′ tracrRNA sequence is at least about 60% identical, about 65% identical, about 70% identical, about 75% identical, about 80% identical, about 85% identical, about 90% identical, about 95% identical, about 98% identical, about 99% identical, or 100% identical, to a reference 3′ tracrRNA sequence (e.g., wild type 3′ tracrRNA sequence from S. pyogenes) over a stretch of at least 6, 7, or 8 contiguous nucleotides.

In some embodiments, a 3′ tracrRNA sequence has more than one duplexed region (e.g., hairpin, hybridized region). In some embodiments, a 3′ tracrRNA sequence has two duplexed regions.

In some embodiments, the 3′ tracrRNA sequence has a stem loop structure. In some embodiments, a stem loop structure in the 3′ tracrRNA has at least 1, 2, 3, 4, 5, 6, 7, 8, 9, 10, 15, or 20 or more nucleotides. In some embodiments, the stem loop structure in the 3′ tracrRNA has at most 1, 2, 3, 4, 5, 6, 7, 8, 9, or 10 or more nucleotides. In some embodiments, the stem loop structure has a functional moiety. For example, the stem loop structure can have an aptamer, a ribozyme, a protein-interacting hairpin, a CRISPR array, an intron, or an exon. In some embodiments, the stem loop structure has at least about 1, 2, 3, 4, or 5 or more functional moieties. In some embodiments, the stem loop structure has at most about 1, 2, 3, 4, or 5 or more functional moieties.

In some embodiments, the hairpin in the 3′ tracrRNA sequence has a P-domain. In some embodiments, the P-domain has a double-stranded region in the hairpin.

tracrRNA Extension Sequence

In some embodiments, a tracrRNA extension sequence can be provided whether the tracrRNA is in the context of single-molecule guides or double-molecule guides. In some embodiments, a tracrRNA extension sequence has a length from about 1 nucleotide to about 400 nucleotides. In some embodiments, a tracrRNA extension sequence has a length of more than 1, 5, 10, 15, 20, 25, 30, 35, 40, 45, 50, 60, 70, 80, 90, 100, 120, 140, 160, 180, 200, 220, 240, 260, 280, 300, 320, 340, 360, 380, or 400 nucleotides. In some embodiments, a tracrRNA extension sequence has a length from about 20 to about 5000 or more nucleotides. In some embodiments, a tracrRNA extension sequence has a length of more than 1000 nucleotides. In some embodiments, a tracrRNA extension sequence has a length of less than 1, 5, 10, 15, 20, 25, 30, 35, 40, 45, 50, 60, 70, 80, 90, 100, 120, 140, 160, 180, 200, 220, 240, 260, 280, 300, 320, 340, 360, 380, 400, or more nucleotides. In some embodiments, a tracrRNA extension sequence can have a length of less than 1000 nucleotides. In some embodiments, a tracrRNA extension sequence has less than 10 nucleotides in length. In some embodiments, a tracrRNA extension sequence is 10-30 nucleotides in length. In some embodiments, tracrRNA extension sequence is 30-70 nucleotides in length.

In some embodiments, the tracrRNA extension sequence has a functional moiety (e.g., a stability control sequence, ribozyme, endoribonuclease binding sequence). In some embodiments, the functional moiety has a transcriptional terminator segment (such as a transcription termination sequence). In some embodiments, the functional moiety has a total length from about 10 nucleotides (nt) to about 100 nucleotides, from about 10 nt to about 20 nt, from about 20 nt to about 30 nt, from about 30 nt to about 40 nt, from about 40 nt to about 50 nt, from about 50 nt to about 60 nt, from about 60 nt to about 70 nt, from about 70 nt to about 80 nt, from about 80 nt to about 90 nt, or from about 90 nt to about 100 nt, from about 15 nt to about 80 nt, from about 15 nt to about 50 nt, from about 15 nt to about 40 nt, from about 15 nt to about 30 nt, or from about 15 nt to about 25 nt. In some embodiments, the functional moiety functions in a eukaryotic cell. In some embodiments, the functional moiety functions in a prokaryotic cell. In some embodiments, the functional moiety functions in both eukaryotic and prokaryotic cells.

Non-limiting examples of suitable tracrRNA extension functional moieties include a 3′ poly-adenylated tail, a riboswitch sequence (e.g., to allow for regulated stability and/or regulated accessibility by proteins and protein complexes), a sequence that forms a dsRNA duplex (such as a hairpin), a sequence that targets the RNA to a subcellular location (e.g., nucleus, mitochondria, chloroplasts, and the like), a modification or sequence that provides for tracking (e.g., direct conjugation to a fluorescent molecule, conjugation to a moiety that facilitates fluorescent detection, a sequence that allows for fluorescent detection, etc.), and/or a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like). In some embodiments, a tracrRNA extension sequence has a primer binding site or a molecular index (e.g., barcode sequence). In some embodiments, the tracrRNA extension sequence has one or more affinity tags.

Single-Molecule Guide Linker Sequence

In some embodiments, the linker sequence of a single-molecule guide nucleic acid has a length from about 3 nucleotides to about 100 nucleotides. In Jinek, M. et al. (2012). Science, 337(6096):816-821, for example, a simple 4 nucleotide “tetraloop” (-GAAA-) was used. An illustrative linker has a length from about 3 nucleotides (nt) to about 90 nt, from about 3 nt to about 80 nt, from about 3 nt to about 70 nt, from about 3 nt to about 60 nt, from about 3 nt to about 50 nt, from about 3 nt to about 40 nt, from about 3 nt to about 30 nt, from about 3 nt to about 20 nt, from about 3 nt to about 10 nt. For example, the linker can have a length from about 3 nt to about 5 nt, from about 5 nt to about 10 nt, from about 10 nt to about 15 nt, from about 15 nt to about 20 nt, from about 20 nt to about 25 nt, from about 25 nt to about 30 nt, from about 30 nt to about 35 nt, from about 35 nt to about 40 nt, from about 40 nt to about 50 nt, from about 50 nt to about 60 nt, from about 60 nt to about 70 nt, from about 70 nt to about 80 nt, from about 80 nt to about 90 nt, or from about 90 nt to about 100 nt. In some embodiments, the linker of a single-molecule guide nucleic acid is between 4 and 40 nucleotides. In some embodiments, a linker is at least about 100, 500, 1000, 1500, 2000, 2500, 3000, 3500, 4000, 4500, 5000, 5500, 6000, 6500, or 7000 or more nucleotides. In some embodiments, a linker is at most about 100, 500, 1000, 1500, 2000, 2500, 3000, 3500, 4000, 4500, 5000, 5500, 6000, 6500, or 7000 or more nucleotides.

Linkers can have any of a variety of sequences, although in some embodiments, the linker will not have sequences that have extensive regions of homology with other portions of the guide RNA, which might cause intramolecular binding that could interfere with other functional regions of the guide. In Jinek, M. et al. (2012). Science, 337(6096):816-821, a simple 4 nucleotide sequence -GAAA- was used, but numerous other sequences, including longer sequences can likewise be used.

In some embodiments, the linker sequence has a functional moiety. For example, the linker sequence can have one or more features, including an aptamer, a ribozyme, a protein-interacting hairpin, a protein binding site, a CRISPR array, an intron, or an exon. In some embodiments, the linker sequence has at least about 1, 2, 3, 4, or 5 or more functional moieties. In some embodiments, the linker sequence has at most about 1, 2, 3, 4, or 5 or more functional moieties.

In some embodiments, a genomic location targeted by gRNAs in accordance with the preset disclosure can be at, within, or near the FOXP3 locus in a genome, e.g., a human genome. Exemplary guide RNAs targeting such locations include the spacer sequences of SEQ ID NOs: 1-7, 15-20, and 27-29. For example, a gRNA including a spacer sequence from SEQ ID NO: 1 can have a spacer sequence including i) the sequence of SEQ ID NO: 1, ii) the sequence from position 2 to position 20 of SEQ ID NO: 1, iii) the sequence from position 3 to position 20 of SEQ ID NO: 1, iv) the sequence from position 4 to position 20 of SEQ ID NO: 1, and so forth. As is understood by the person of ordinary skill in the art, each guide RNA is designed to include a spacer sequence complementary to its genomic target sequence. For example, each of the spacer sequences of SEQ ID NOs: 1-7, 15-20, and 27-29 can be put into a single RNA chimera or a crRNA (along with a corresponding tracrRNA). See Jinek, M. et al. (2012) Science, 337(6096):816-821, and Deltcheva, E. et al. (2011) Nature, 471:602-607.

Donor DNA or Donor Template

Site-directed polypeptides, such as a DNA endonuclease, can introduce double-strand breaks or single-strand breaks in nucleic acids, e.g., genomic DNA. The double-strand break can stimulate a cell's endogenous DNA-repair pathways (e.g., homology-dependent repair (HDR) or non-homologous end joining or alternative non-homologous end joining (A-NHEJ) or microhomology-mediated end joining (MMEJ). NHEJ can repair cleaved target nucleic acid without the need for a homologous template. This can sometimes result in small deletions or insertions (indels) in the target nucleic acid at the site of cleavage and can lead to disruption or alteration of gene expression. HDR, which is also known as homologous recombination (HR) can occur when a homologous repair template, or donor, is available.

The homologous donor template has sequences that are homologous to sequences flanking the target nucleic acid cleavage site. The sister chromatid is generally used by the cell as the repair template. However, for the purposes of genome editing, the repair template is often supplied as an exogenous nucleic acid, such as a plasmid, duplex oligonucleotide, single-strand oligonucleotide, double-stranded oligonucleotide, or viral nucleic acid. With exogenous donor templates, it is common to introduce an additional nucleic acid sequence (such as a transgene) or modification (such as a single or multiple base change or a deletion) between the flanking regions of homology so that the additional or altered nucleic acid sequence also becomes incorporated into the target locus. MMEJ results in a genetic outcome that is similar to NHEJ in that small deletions and insertions can occur at the cleavage site. MMEJ makes use of homologous sequences of a few base pairs flanking the cleavage site to drive a favored end-joining DNA repair outcome. In some instances, it can be possible to predict likely repair outcomes based on analysis of potential microhomologies in the nuclease target regions.

Thus, in some cases, homologous recombination is used to insert an exogenous polynucleotide sequence into the target nucleic acid cleavage site. An exogenous polynucleotide sequence is termed a donor polynucleotide (or donor or donor sequence or polynucleotide donor template) herein. In some embodiments, the donor polynucleotide, a portion of the donor polynucleotide, a copy of the donor polynucleotide, or a portion of a copy of the donor polynucleotide is inserted into the target nucleic acid cleavage site. In some embodiments, the donor polynucleotide is an exogenous polynucleotide sequence, such as a sequence that does not naturally occur at the target nucleic acid cleavage site.

When an exogenous DNA molecule is supplied in sufficient concentration inside the nucleus of a cell in which the double-strand break occurs, the exogenous DNA can be inserted at the double-strand break during the NHEJ repair process and thus become a permanent addition to the genome. These exogenous DNA molecules are referred to as donor templates in some embodiments. If the donor template contains a coding sequence for a gene of interest such as a FOXP3 gene optionally together with relevant regulatory sequences such as promoters, enhancers, polyA sequences and/or splice acceptor sequences (also referred to herein as a “donor cassette”), the gene of interest can be expressed from the integrated copy in the genome resulting in permanent expression for the life of the cell. Moreover, the integrated copy of the donor DNA template can be transmitted to the daughter cells when the cell divides.

In the presence of sufficient concentrations of a donor DNA template that contains flanking DNA sequences with homology to the DNA sequence either side of the double-strand break (referred to as homology arms), the donor DNA template can be integrated via the HDR pathway. The homology arms act as substrates for homologous recombination between the donor template and the sequences either side of the double-strand break. This can result in an error-free insertion of the donor template in which the sequences either side of the double-strand break are not altered from that in the unmodified genome.

Supplied donors for editing by HDR vary markedly but generally contain the intended sequence with small or large flanking homology arms to allow annealing to the genomic DNA. The homology regions flanking the introduced genetic changes can be 30 bp or smaller, or as large as a multi-kilobase cassette that can contain promoters, cDNAs, etc. Both single-stranded and double-stranded oligonucleotide donors can be used. These oligonucleotides range in size from less than 100 nt to over many kb, though longer ssDNA can also be generated and used. Double-stranded donors are often used, including PCR amplicons, plasmids, and mini-circles. In general, it has been found that an AAV vector is a very effective means of delivery of a donor template, though the packaging limits for individual donors is <5 kb. Active transcription of the donor increased HDR three-fold, indicating the inclusion of promoter can increase conversion. Conversely, CpG methylation of the donor can decrease gene expression and HDR.

In some embodiments, the donor DNA can be supplied with the nuclease or independently by a variety of different methods, for example by transfection, nanoparticle, micro-injection, or viral transduction. A range of tethering options can be used to increase the availability of the donors for HDR in some embodiments. Examples include attaching the donor to the nuclease, attaching to DNA binding proteins that bind nearby, or attaching to proteins that are involved in DNA end binding or repair.

In addition to genome editing by MEI or HDR, site-specific gene insertions can be conducted that use both the MEI pathway and HR. A combination approach can be applicable in certain settings, possibly including intron/exon borders. MEI can prove effective for ligation in the intron, while the error-free HDR can be better suited in the coding region.

In some embodiments, an exogenous sequence that is intended to be inserted into a genome is a nucleotide sequence encoding a FOXP3 or a functional derivative thereof. The functional derivative of a FOXP3 can include a derivative of the FOXP3 that has a substantial activity of a wild-type FOXP3, such as the wild-type human FOXP3, e.g., at least at or about 30%, about 40%, about 50%, about 60%, about 70%, about 80%, about 90%, about 95% or about 100% of the activity that the wild-type FOXP3 exhibits. In some embodiments, the functional derivative of a FOXP3 can have at least about 30%, about 40%, about 50%, about 60%, about 70%, about 80%, about 85%, about 90%, about 95%, about 96%, about 97%, about 98% or about 99% amino acid sequence identity to the FOXP3, e.g., the wild-type FOXP3. In some embodiments, one having ordinary skill in the art can use a number of methods known in the field to test the functionality or activity of a compound, e.g., a peptide or protein. The functional derivative of the FOXP3 can also include any fragment of the wild-type FOXP3 or fragment of a modified FOXP3 that has conservative modification on one or more of amino acid residues in the full length, wild-type FOXP3. Thus, in some embodiments, a nucleic acid sequence encoding a functional derivative of a FOXP3 can have at least about 30%, about 40%, about 50%, about 60%, about 70%, about 80%, about 85%, about 90%, about 95%, about 96%, about 97%, about 98% or about 99% nucleic acid sequence identity to a nucleic acid sequence encoding the FOXP3, e.g., the wild-type FOXP3. In some embodiments, the FOXP3 is a human wild-type FOXP3.

In some embodiments where the insertion of a nucleic acid encoding a FOXP3 or a functional derivative thereof is concerned, a cDNA of the FOXP3gene or a functional derivative thereof can be inserted into a genome of a subject having a defective FOXP3 gene or its regulatory sequences. In such a case, a donor DNA or donor template can be an expression cassette or vector construct having a sequence encoding the FOXP3 or a functional derivative thereof, e.g., a cDNA sequence.

In some embodiments, according to any of the donor templates described herein comprising a donor cassette, the donor cassette is flanked on one or both sides by a gRNA target site. For example, such a donor template may comprise a donor cassette with a gRNA target site 5′ of the donor cassette and/or a gRNA target site 3′ of the donor cassette. In some embodiments, the donor template comprises a donor cassette with a gRNA target site 5′ of the donor cassette. In some embodiments, the donor template comprises a donor cassette with a gRNA target site 3′ of the donor cassette. In some embodiments, the donor template comprises a donor cassette with a gRNA target site 5′ of the donor cassette and a gRNA target site 3′ of the donor cassette. In some embodiments, the donor template comprises a donor cassette with a gRNA target site 5′ of the donor cassette and a gRNA target site 3′ of the donor cassette, and the two gRNA target sites comprise the same sequence. In some embodiments, the donor template comprises at least one gRNA target site, and the at least one gRNA target site in the donor template comprises the same sequence as a gRNA target site in a target locus into which the donor cassette of the donor template is to be integrated. In some embodiments, the donor template comprises at least one gRNA target site, and the at least one gRNA target site in the donor template comprises the reverse complement of a gRNA target site in a target locus into which the donor cassette of the donor template is to be integrated. In some embodiments, the donor template comprises a donor cassette with a gRNA target site 5′ of the donor cassette and a gRNA target site 3′ of the donor cassette, and the two gRNA target sites in the donor template comprises the same sequence as a gRNA target site in a target locus into which the donor cassette of the donor template is to be integrated. In some embodiments, the donor template comprises a donor cassette with a gRNA target site 5′ of the donor cassette and a gRNA target site 3′ of the donor cassette, and the two gRNA target sites in the donor template comprises the reverse complement of a gRNA target site in a target locus into which the donor cassette of the donor template is to be integrated.

In some embodiments, provided herein is a donor template comprising a nucleotide sequence encoding a FOXP3 or a functional derivative thereof for targeted integration into a FOXP3 locus, wherein the donor template comprises, from 5′ to 3′, i) a first gRNA target site; ii) a splice acceptor; iii) the nucleotide sequence encoding a FOXP3 or a functional derivative thereof; and iv) a polyadenylation signal. In some embodiments, the donor template further comprises a second gRNA target site downstream of the iv) polyadenylation signal. In some embodiments, the first gRNA target site and the second gRNA target site are the same. In some embodiments, the donor template further comprises a polynucleotide spacer between the i) first gRNA target site and the ii) splice acceptor. In some embodiments, the polynucleotide spacer is 18 nucleotides in length. In some embodiments, the donor template is flanked on one side by a first AAV ITR and/or flanked on the other side by a second AAV ITR. In some embodiments, the first AAV ITR is an AAV2 ITR and/or the second AAV ITR is an AAV2 ITR. In some embodiments, the FOXP3 is a human wild-type FOXP3.

Nucleic Acid Encoding a Site-Directed Polypeptide or DNA Endonuclease

In some embodiments, the methods of genome edition and compositions therefore can use a nucleic acid sequence (or oligonucleotide) encoding a site-directed polypeptide or DNA endonuclease. The nucleic acid sequence encoding the site-directed polypeptide can be DNA or RNA. If the nucleic acid sequence encoding the site-directed polypeptide is RNA, it can be covalently linked to a gRNA sequence or exist as a separate sequence. In some embodiments, a peptide sequence of the site-directed polypeptide or DNA endonuclease can be used instead of the nucleic acid sequence thereof.

Vectors

In another aspect, the present disclosure provides a nucleic acid having a nucleotide sequence encoding a genome-targeting nucleic acid of the disclosure, a site-directed polypeptide of the disclosure, and/or any nucleic acid or proteinaceous molecule necessary to carry out the embodiments of the methods of the disclosure. In some embodiments, such a nucleic acid is a vector (e.g., a recombinant expression vector).

Expression vectors contemplated include, but are not limited to, viral vectors based on vaccinia virus, poliovirus, adenovirus, adeno-associated virus, SV40, herpes simplex virus, human immunodeficiency virus, retrovirus (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus) and other recombinant vectors. Other vectors contemplated for eukaryotic target cells include, but are not limited to, the vectors pXT1, pSG5, pSVK3, pBPV, pMSG, and pSVLSV40 (Pharmacia). Additional vectors contemplated for eukaryotic target cells include, but are not limited to, the vectors pCTx-1, pCTx-2, and pCTx-3. Other vectors can be used so long as they are compatible with the host cell.

In some embodiments, a vector has one or more transcription and/or translation control elements. Depending on the host/vector system utilized, any of a number of suitable transcription and translation control elements, including constitutive and inducible promoters, transcription enhancer elements, transcription terminators, etc. can be used in the expression vector. In some embodiments, the vector is a self-inactivating vector that either inactivates the viral sequences or the components of the CRISPR machinery or other elements.

Non-limiting examples of suitable eukaryotic promoters (such as promoters functional in a eukaryotic cell) include those from cytomegalovirus (CMV) immediate early, herpes simplex virus (HSV) thymidine kinase, early and late SV40, long terminal repeats (LTRs) from retrovirus, human elongation factor-1 promoter (EF1), a hybrid construct having the cytomegalovirus (CMV) enhancer fused to the chicken beta-actin promoter (CAG), murine stem cell virus promoter (MSCV), phosphoglycerate kinase-1 locus promoter (PGK), and mouse metallothionein-I.

For expressing small RNAs, including guide RNAs used in connection with Cas endonuclease, various promoters such as RNA polymerase III promoters, including for example U6 and H1, can be advantageous. Descriptions of and parameters for enhancing the use of such promoters are known in art, and additional information and approaches are regularly being described; see, e.g., Ma, H. et al. (2014). Molecular Therapy—Nucleic Acids 3:e161, doi:10.1038/mtna.2014.12.

The expression vector can also contain a ribosome binding site for translation initiation and a transcription terminator. The expression vector can also include appropriate sequences for amplifying expression. The expression vector can also include nucleotide sequences encoding non-native tags (e.g., histidine tag, hemagglutinin tag, green fluorescent protein, etc.) that are fused to the site-directed polypeptide, thus resulting in a fusion protein.

In some embodiments, a promoter is an inducible promoter (e.g., a heat shock promoter, tetracycline-regulated promoter, steroid-regulated promoter, metal-regulated promoter, estrogen receptor-regulated promoter, etc.). In some embodiments, a promoter is a constitutive promoter (e.g., CMV promoter, UBC promoter). In some embodiments, the promoter is a spatially restricted and/or temporally restricted promoter (e.g., a tissue specific promoter, a cell type specific promoter, etc.). In some embodiments, a vector does not have a promoter for at least one gene to be expressed in a host cell if the gene is going to be expressed, after it is inserted into a genome, under an endogenous promoter present in the genome.

In some embodiments, a first vector can encode a first CISC component comprising a first extracellular binding domain or portion thereof, a hinge domain, a transmembrane domain, and a signaling domain or portion thereof while a second vector can encode a second CISC component comprising a second extracellular binding domain or a portion thereof, a hinge domain, a transmembrane domain, and a signaling domain or portions thereof.

In some embodiments, the expression vector comprises a nucleic acid encoding the protein sequence of any one of SEQ ID NOs: 48-61. In some embodiments, the expression vector comprises a nucleic acid sequence as set forth in SEQ ID NO: 67. SEQ ID NO: 67 encodes the protein sequences as set forth in SEQ ID NO: 54.

In some embodiments, the expression vector is a variant of SEQ ID NO: 67 as set forth in SEQ ID NO: 65. SEQ ID NO: 65 encodes the protein sequences as set forth in SEQ ID NOs: 50 and 51.

In some embodiments, the expression vector is a variant of SEQ ID NO: 67 as set forth in SEQ ID NO: 66. SEQ ID NO: 66 encodes the protein sequences as set forth in SEQ ID NOs: 52 and 53.

In some embodiments, the expression vector includes a nucleic acid having at least 80%, 85%, 90%, 95%, 98% or 99% nucleic acid sequence identity (or a percentage nucleic acid sequence identity within a range defined by any two of the aforementioned percentages) with the nucleotide sequences provided herein, or a specifically derived fragment thereof. In some embodiments, the expression vector comprises a promoter. In some embodiments, the expression vector comprises the nucleic acid encoding a fusion protein. In some embodiments, the vector is RNA or DNA.

### Site-Directed Polypeptide or DNA Endonuclease

Modifications of a target DNA due to NHEJ and/or HDR can lead to, for example, mutations, deletions, alterations, integrations, gene correction, gene replacement, gene tagging, transgene insertion, nucleotide deletion, gene disruption, translocations, and/or gene mutation. The process of integrating non-native nucleic acid into genomic DNA is an example of genome editing.

A site-directed polypeptide is a nuclease used in genome editing to cleave DNA. The site-directed polypeptide can be administered to a cell or a subject as either: one or more polypeptides, or one or more mRNAs encoding the polypeptide.

In the context of a CRISPR/Cas or CRISPR/Cpf1 system, the site-directed polypeptide can bind to a guide RNA that, in turn, specifies the site in the target DNA to which the polypeptide is directed. In embodiments of CRISPR/Cas or CRISPR/Cpf1 systems herein, the site-directed polypeptide is an endonuclease, such as a DNA endonuclease.

In some embodiments, a site-directed polypeptide has a plurality of nucleic acid-cleaving (such as nuclease) domains. Two or more nucleic acid-cleaving domains can be linked together via a linker. In some embodiments, the linker has a flexible linker. Linkers can have 1, 2, 3, 4, 5, 6, 7, 8, 9, 10, 11, 12, 13, 14, 15, 16, 17, 18, 19, 20, 21, 22, 23, 24, 25, 30, 35, 40, or more amino acids in length.

Naturally-occurring wild-type Cas9 enzymes have two nuclease domains, an HNH nuclease domain and a RuvC domain. Cas9 enzymes contemplated herein have an HNH or HNH-like nuclease domain, and/or a RuvC or RuvC-like nuclease domain.

HNH or HNH-like domains have a McrA-like fold. HNH or HNH-like domains has two antiparallel β-strands and an α-helix. HNH or HNH-like domains has a metal binding site (e.g., a divalent cation binding site). HNH or HNH-like domains can cleave one strand of a target nucleic acid (e.g., the complementary strand of the crRNA targeted strand).

RuvC or RuvC-like domains have an RNaseH or RNaseH-like fold. RuvC/RNaseH domains are involved in a diverse set of nucleic acid-based functions including acting on both RNA and DNA. The RNaseH domain has 5 β-strands surrounded by a plurality of α-helices. RuvC/RNaseH or RuvC/RNaseH-like domains have a metal binding site (e.g., a divalent cation binding site). RuvC/RNaseH or RuvC/RNaseH-like domains can cleave one strand of a target nucleic acid (e.g., the non-complementary strand of a double-stranded target DNA).

In some embodiments, the site-directed polypeptide has an amino acid sequence having at least 10%, at least 15%, at least 20%, at least 30%, at least 40%, at least 50%, at least 60%, at least 70%, at least 75%, at least 80%, at least 85%, at least 90%, at least 95%, at least 99%, or 100% amino acid sequence identity to a wild-type exemplary site-directed polypeptide [e.g., Cas9 from S. pyogenes, US 2014/0068797 Sequence ID No. 8 or Sapranauskas, R. et al. (2011). Nucleic Acids Res, 39(21):9275-9282], and various other site-directed polypeptides).

In some embodiments, the site-directed polypeptide has an amino acid sequence having at least 10%, at least 15%, at least 20%, at least 30%, at least 40%, at least 50%, at least 60%, at least 70%, at least 75%, at least 80%, at least 85%, at least 90%, at least 95%, at least 99%, or 100% amino acid sequence identity to the nuclease domain of a wild-type exemplary site-directed polypeptide (e.g., Cas9 from S. pyogenes, supra).

In some embodiments, a site-directed polypeptide has at least 70, 75, 80, 85, 90, 95, 97, 99, or 100% identity to a wild-type site-directed polypeptide (e.g., Cas9 from S. pyogenes, supra) over 10 contiguous amino acids. In some embodiments, a site-directed polypeptide has at most: 70, 75, 80, 85, 90, 95, 97, 99, or 100% identity to a wild-type site-directed polypeptide (e.g., Cas9 from S. pyogenes, supra) over 10 contiguous amino acids. In some embodiments, a site-directed polypeptide has at least: 70, 75, 80, 85, 90, 95, 97, 99, or 100% identity to a wild-type site-directed polypeptide (e.g., Cas9 from S. pyogenes, supra) over 10 contiguous amino acids in an HNH nuclease domain of the site-directed polypeptide. In some embodiments, a site-directed polypeptide has at most: 70, 75, 80, 85, 90, 95, 97, 99, or 100% identity to a wild-type site-directed polypeptide (e.g., Cas9 from S. pyogenes, supra) over 10 contiguous amino acids in an HNH nuclease domain of the site-directed polypeptide. In some embodiments, a site-directed polypeptide has at least: 70, 75, 80, 85, 90, 95, 97, 99, or 100% identity to a wild-type site-directed polypeptide (e.g., Cas9 from S. pyogenes, supra) over 10 contiguous amino acids in a RuvC nuclease domain of the site-directed polypeptide. In some embodiments, a site-directed polypeptide has at most: 70, 75, 80, 85, 90, 95, 97, 99, or 100% identity to a wild-type site-directed polypeptide (e.g., Cas9 from S. pyogenes, supra) over 10 contiguous amino acids in a RuvC nuclease domain of the site-directed polypeptide.

In some embodiments, the site-directed polypeptide has a modified form of a wild-type exemplary site-directed polypeptide. The modified form of the wild-type exemplary site-directed polypeptide has a mutation that reduces the nucleic acid-cleaving activity of the site-directed polypeptide. In some embodiments, the modified form of the wild-type exemplary site-directed polypeptide has less than 90%, less than 80%, less than 70%, less than 60%, less than 50%, less than 40%, less than 30%, less than 20%, less than 10%, less than 5%, or less than 1% of the nucleic acid-cleaving activity of the wild-type exemplary site-directed polypeptide (e.g., Cas9 from S. pyogenes, supra). The modified form of the site-directed polypeptide can have no substantial nucleic acid-cleaving activity. When a site-directed polypeptide is a modified form that has no substantial nucleic acid-cleaving activity, it is referred to herein as “enzymatically inactive.”

In some embodiments, the modified form of the site-directed polypeptide has a mutation such that it can induce a single-strand break (SSB) on a target nucleic acid (e.g., by cutting only one of the sugar-phosphate backbones of a double-strand target nucleic acid). In some embodiments, the mutation results in less than 90%, less than 80%, less than 70%, less than 60%, less than 50%, less than 40%, less than 30%, less than 20%, less than 10%, less than 5%, or less than 1% of the nucleic acid-cleaving activity in one or more of the plurality of nucleic acid-cleaving domains of the wild-type site directed polypeptide (e.g., Cas9 from S. pyogenes, supra). In some embodiments, the mutation results in one or more of the plurality of nucleic acid-cleaving domains retaining the ability to cleave the complementary strand of the target nucleic acid, but reducing its ability to cleave the non-complementary strand of the target nucleic acid. In some embodiments, the mutation results in one or more of the plurality of nucleic acid-cleaving domains retaining the ability to cleave the non-complementary strand of the target nucleic acid, but reducing its ability to cleave the complementary strand of the target nucleic acid. For example, residues in the wild-type exemplary S. pyogenes Cas9 polypeptide, such as Asp10, His840, Asn854, and Asn856, are mutated to inactivate one or more of the plurality of nucleic acid-cleaving domains (e.g., nuclease domains). In some embodiments, the residues to be mutated correspond to residues Asp10, His840, Asn854, and Asn856 in the wild-type exemplary S. pyogenes Cas9 polypeptide (e.g., as determined by sequence and/or structural alignment). Non-limiting examples of mutations include D10A, H840A, N854A, or N856A. One skilled in the art will recognize that mutations other than alanine substitutions are suitable.

In some embodiments, a D10A mutation is combined with one or more of H840A, N854A, or N856A mutations to produce a site-directed polypeptide substantially lacking DNA cleavage activity. In some embodiments, a H840A mutation is combined with one or more of D10A, N854A, or N856A mutations to produce a site-directed polypeptide substantially lacking DNA cleavage activity. In some embodiments, a N854A mutation is combined with one or more of H840A, D10A, or N856A mutations to produce a site-directed polypeptide substantially lacking DNA cleavage activity. In some embodiments, a N856A mutation is combined with one or more of H840A, N854A, or D10A mutations to produce a site-directed polypeptide substantially lacking DNA cleavage activity. Site-directed polypeptides that have one substantially inactive nuclease domain are referred to as “nickases”.

In some embodiments, variants of RNA-guided endonucleases, for example Cas9, can be used to increase the specificity of CRISPR-mediated genome editing. Wild type Cas9 is generally guided by a single guide RNA designed to hybridize with a specified ˜20 nucleotide sequence in the target sequence (such as an endogenous genomic locus). However, several mismatches can be tolerated between the guide RNA and the target locus, effectively reducing the length of required homology in the target site to, for example, as little as 13 nt of homology, and thereby resulting in elevated potential for binding and double-strand nucleic acid cleavage by the CRISPR/Cas9 complex elsewhere in the target genome—also known as off-target cleavage. Because nickase variants of Cas9 each only cut one strand, to create a double-strand break it is necessary for a pair of nickases to bind in close proximity and on opposite strands of the target nucleic acid, thereby creating a pair of nicks, which is the equivalent of a double-strand break. This requires that two separate guide RNAs—one for each nickase—must bind in close proximity and on opposite strands of the target nucleic acid. This requirement essentially doubles the minimum length of homology needed for the double-strand break to occur, thereby reducing the likelihood that a double-strand cleavage event will occur elsewhere in the genome, where the two guide RNA sites—if they exist—are unlikely to be sufficiently close to each other to enable the double-strand break to form. As described in the art, nickases can also be used to promote HDR versus NHEJ. HDR can be used to introduce selected changes into target sites in the genome through the use of specific donor sequences that effectively mediate the desired changes. Descriptions of various CRISPR/Cas systems for use in gene editing can be found, e.g., in International Patent Application no. WO 2013/176772, and in Sander, J. D. et al. (2014). Nature Biotechnology 32(4):347-355, and references cited therein.

In some embodiments, the site-directed polypeptide (e.g., variant, mutated, enzymatically inactive and/or conditionally enzymatically inactive site-directed polypeptide) targets nucleic acid. In some embodiments, the site-directed polypeptide (e.g., variant, mutated, enzymatically inactive and/or conditionally enzymatically inactive endoribonuclease) targets DNA. In some embodiments, the site-directed polypeptide (e.g., variant, mutated, enzymatically inactive and/or conditionally enzymatically inactive endoribonuclease) targets RNA.

In some embodiments, the site-directed polypeptide has one or more non-native sequences (e.g., the site-directed polypeptide is a fusion protein).

In some embodiments, the site-directed polypeptide has an amino acid sequence having at least 15% amino acid identity to a Cas9 from a bacterium (e.g., S. pyogenes), a nucleic acid binding domain, and two nucleic acid cleaving domains (such as an HNH domain and a RuvC domain).

In some embodiments, the site-directed polypeptide has an amino acid sequence having at least 15% amino acid identity to a Cas9 from a bacterium (e.g., S. pyogenes), and two nucleic acid cleaving domains (such as an HNH domain and a RuvC domain).

In some embodiments, the site-directed polypeptide has an amino acid sequence having at least 15% amino acid identity to a Cas9 from a bacterium (e.g., S. pyogenes), and two nucleic acid cleaving domains, wherein one or both of the nucleic acid cleaving domains have at least 50% amino acid identity to a nuclease domain from Cas9 from a bacterium (e.g., S. pyogenes).

In some embodiments, the site-directed polypeptide has an amino acid sequence having at least 15% amino acid identity to a Cas9 from a bacterium (e.g., S. pyogenes), two nucleic acid cleaving domains (such as an HNH domain and a RuvC domain), and non-native sequence (for example, a nuclear localization signal) or a linker linking the site-directed polypeptide to a non-native sequence.

In some embodiments, the site-directed polypeptide has an amino acid sequence having at least 15% amino acid identity to a Cas9 from a bacterium (e.g., S. pyogenes), two nucleic acid cleaving domains (such as an HNH domain and a RuvC domain), wherein the site-directed polypeptide has a mutation in one or both of the nucleic acid cleaving domains that reduces the cleaving activity of the nuclease domains by at least 50%.

In some embodiments, the site-directed polypeptide has an amino acid sequence having at least 15% amino acid identity to a Cas9 from a bacterium (e.g., S. pyogenes), and two nucleic acid cleaving domains (such as an HNH domain and a RuvC domain), wherein one of the nuclease domains has mutation of aspartic acid 10, and/or wherein one of the nuclease domains has mutation of histidine 840, and wherein the mutation reduces the cleaving activity of the nuclease domain(s) by at least 50%.

In some embodiments, the one or more site-directed polypeptides, e.g., DNA endonucleases, include two nickases that together effect one double-strand break at a specific locus in the genome, or four nickases that together effect two double-strand breaks at specific loci in the genome. Alternatively, one site-directed polypeptide, e.g., DNA endonuclease, affects one double-strand break at a specific locus in the genome.

In some embodiments, a polynucleotide encoding a site-directed polypeptide can be used to edit genome. In some of such embodiments, the polynucleotide encoding a site-directed polypeptide is codon-optimized according to methods known in the art for expression in the cell containing the target DNA of interest. For example, if the intended target nucleic acid is in a human cell, a human codon-optimized polynucleotide encoding Cas9 is contemplated for use for producing the Cas9 polypeptide.

The following provides some examples of site-directed polypeptides that can be used in various embodiments of the disclosures.

CRISPR Endonuclease System

A CRISPR (Clustered Regularly Interspaced Short Palindromic Repeats) genomic locus can be found in the genomes of many prokaryotes (e.g., bacteria and archaea). In prokaryotes, the CRISPR locus encodes products that function as a type of immune system to help defend the prokaryotes against foreign invaders, such as virus and phage. There are three stages of CRISPR locus function: integration of new sequences into the CRISPR locus, expression of CRISPR RNA (crRNA), and silencing of foreign invader nucleic acid. Five types of CRISPR systems (e.g., Type I, Type II, Type III, Type U, and Type V) have been identified.

A CRISPR locus includes a number of short repeating sequences referred to as “repeats.” When expressed, the repeats can form secondary hairpin structures (e.g., hairpins) and/or unstructured single-stranded sequences. The repeats usually occur in clusters and frequently diverge between species. The repeats are regularly interspaced with unique intervening sequences referred to as “spacers,” resulting in a repeat-spacer-repeat locus architecture. The spacers are identical to or have high homology with known foreign invader sequences. A spacer-repeat unit encodes a crisprRNA (crRNA), which is processed into a mature form of the spacer-repeat unit. A crRNA has a “seed” or spacer sequence that is involved in targeting a target nucleic acid (in the naturally occurring form in prokaryotes, the spacer sequence targets the foreign invader nucleic acid). A spacer sequence is located at the 5′ or 3′ end of the crRNA.

A CRISPR locus also has polynucleotide sequences encoding CRISPR Associated (Cas) genes. Cas genes encode endonucleases involved in the biogenesis and the interference stages of crRNA function in prokaryotes. Some Cas genes have homologous secondary and/or tertiary structures.

Type II CRISPR Systems

crRNA biogenesis in a Type II CRISPR system in nature requires a trans-activating CRISPR RNA (tracrRNA). The tracrRNA is modified by endogenous RNaseIII, and then hybridizes to a crRNA repeat in the pre-crRNA array. Endogenous RNaseIII is recruited to cleave the pre-crRNA. Cleaved crRNAs are subjected to exoribonuclease trimming to produce the mature crRNA form (e.g., 5′ trimming). The tracrRNA remains hybridized to the crRNA, and the tracrRNA and the crRNA associate with a site-directed polypeptide (e.g., Cas9). The crRNA of the crRNA-tracrRNA-Cas9 complex guides the complex to a target nucleic acid to which the crRNA can hybridize. Hybridization of the crRNA to the target nucleic acid activates Cas9 for targeted nucleic acid cleavage. The target nucleic acid in a Type II CRISPR system is referred to as a protospacer adjacent motif (PAM). In nature, the PAM is essential to facilitate binding of a site-directed polypeptide (e.g., Cas9) to the target nucleic acid. Type II systems (also referred to as Nmeni or CASS4) are further subdivided into Type II-A (CASS4) and II-B (CASS4a). Jinek, M. et al. (2012). Science, 337(6096):816-821 showed that the CRISPR/Cas9 system is useful for RNA-programmable genome editing, and International Patent Application no. WO 2013/176772 provides numerous examples and applications of the CRISPR/Cas endonuclease system for site-specific gene editing.

Type V CRISPR Systems

Type V CRISPR systems have several important differences from Type II systems. For example, Cpf1 is a single RNA-guided endonuclease that, in contrast to Type II systems, lacks tracrRNA. In fact, Cpf1-associated CRISPR arrays are processed into mature crRNAs without the requirement of an additional trans-activating tracrRNA. The Type V CRISPR array is processed into short mature crRNAs of 42-44 nucleotides in length, with each mature crRNA beginning with 19 nucleotides of direct repeat followed by 23-25 nucleotides of spacer sequence. In contrast, mature crRNAs in Type II systems start with 20-24 nucleotides of spacer sequence followed by at or about 22 nucleotides of direct repeat. Also, Cpf1 utilizes a T-rich protospacer-adjacent motif such that Cpf1-crRNA complexes efficiently cleave target DNA preceded by a short T-rich PAM, which is in contrast to the G-rich PAM following the target DNA for Type II systems. Thus, Type V systems cleave at a point that is distant from the PAM, while Type II systems cleave at a point that is adjacent to the PAM. In addition, in contrast to Type II systems, Cpf1 cleaves DNA via a staggered DNA double-stranded break with a 4 or 5 nucleotide 5′ overhang. Type II systems cleave via a blunt double-stranded break. Similar to Type II systems, Cpf1 contains a predicted RuvC-like endonuclease domain, but lacks a second HNH endonuclease domain, which is in contrast to Type II systems.

Cas Genes/Polypeptides and Protospacer Adjacent Motifs

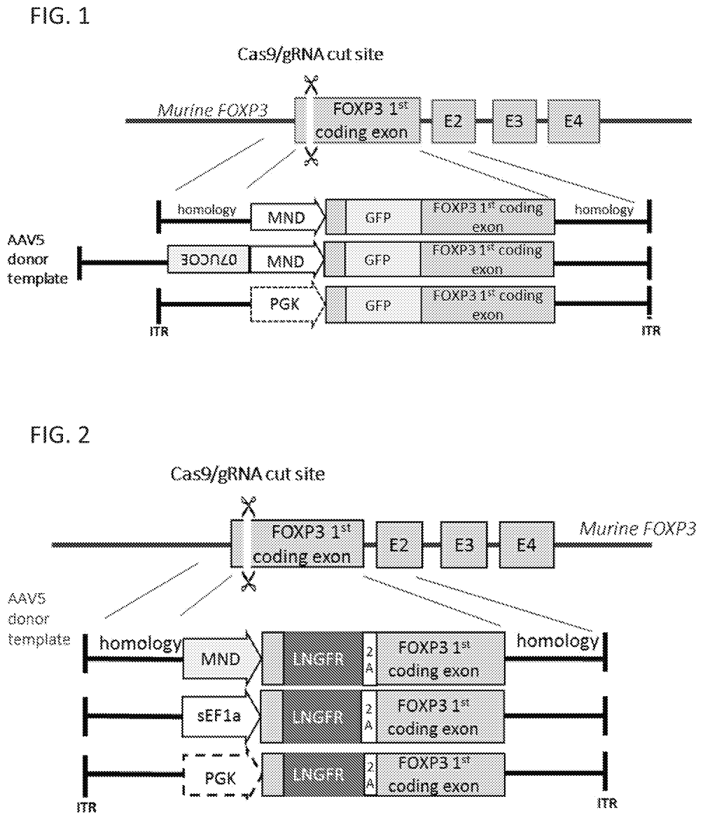

Exemplary CRISPR/Cas polypeptides include the Cas9 polypeptides in FIG. 1 of Fonfara, I. et al. (2014). Nucleic Acids Research, 42(4):2577-2590. The CRISPR/Cas gene naming system has undergone extensive rewriting since the Cas genes were discovered. FIG. 5 of Fonfara et al. (2014) provides PAM sequences for the Cas9 polypeptides from various species.

Complexes of a Genome-Targeting Nucleic acid and a Site-Directed Polypeptide

A genome-targeting nucleic acid interacts with a site-directed polypeptide (e.g., a nucleic acid-guided nuclease such as Cas9), thereby forming a complex. The genome-targeting nucleic acid (e.g., gRNA) guides the site-directed polypeptide to a target nucleic acid.

As stated previously, in some embodiments the site-directed polypeptide and genome-targeting nucleic acid can each be administered separately to a cell or a subject. On the other hand, in some other embodiments the site-directed polypeptide can be pre-complexed with one or more guide RNAs, or one or more crRNA together with a tracrRNA. The pre-complexed material can then be administered to a cell or a subject. Such pre-complexed material is known as a ribonucleoprotein particle (RNP).

CISC Components

As described herein, in some embodiments, one or more protein sequences encoding a dimeric CISC component is provided. The one or more protein sequence can have a first and a second sequence. In some embodiments, a first sequence encodes a first CISC component that can comprise a first extracellular binding domain or portion thereof, a hinge domain, a transmembrane domain, and a signaling domain or portion thereof. In some embodiments, a second sequence encodes a second CISC component that can comprise a second extracellular binding domain or a portion thereof, a hinge domain, a transmembrane domain, and a signaling domain or portions thereof. In some embodiments, the first and second CISC components may be positioned such that when expressed, they dimerize in the presence of a ligand, preferably simultaneously.

In some embodiments, a protein sequence or sequences for heterodimeric two component CISC are provided. In some embodiments, the first CISC component is an IL2Rγ-CISC complex.

In some embodiments, the IL2Rγ-CISC comprises an amino acid sequence as set forth in SEQ ID NO: 48. Embodiments also comprise a nucleic acid sequence encoding the protein sequence of SEQ ID NO: 48.

In some embodiments, the IL2Rγ-CISC comprises an amino acid sequence as set forth in SEQ ID NO: 50. Embodiments also comprise a nucleic acid sequence encoding the protein sequence of SEQ ID NO: 50.

In some embodiments, the IL2Rγ-CISC comprises an amino acid sequence as set forth in SEQ ID NO: 52. Embodiments also comprise a nucleic acid sequence encoding the protein sequence of SEQ ID NO: 52.

In some embodiments, the IL2Rγ-CISC comprises an amino acid sequence as set forth in SEQ ID NO: 54. Embodiments also comprise a nucleic acid sequence encoding the protein sequence of SEQ ID NO: 54.

In some embodiments, the protein sequence for the first CISC component includes a protein sequence encoding an extracellular binding domain, a hinge domain, a transmembrane domain, or a signaling domain. Embodiments also comprise a nucleic acid sequence encoding the extracellular binding domain, the hinge domain, the transmembrane domain, or the signaling domain. In some embodiments, the protein sequence of the first CISC component, comprising the first extracellular binding domain, the hinge domain, the transmembrane domain, and/or the signaling domain comprises an amino acid sequence that comprises a 100%, 99%, 98%, 95%, 90%, 85%, or 80% sequence identity to the sequence set forth in SEQ ID NOs: 48, 50, 52, or 54, or has a sequence identity that is within a range defined by any two of the aforementioned percentages.

In some embodiments, the second CISC component is an IL2Rβ complex. In some embodiments, the IL2Rβ-CISC comprises an amino acid sequence as set forth in SEQ ID NO: 49. Embodiments also comprise a nucleic acid sequence encoding the protein sequence of SEQ ID NO: 49.

In some embodiments, the IL2Rβ-CISC comprises an amino acid sequence as set forth in SEQ ID NO: 51. Embodiments also comprise a nucleic acid sequence encoding the protein sequence of SEQ ID NO: 51.

In some embodiments, the IL2Rβ-CISC comprises an amino acid sequence as set forth in SEQ ID NO: 53. Embodiments also comprise a nucleic acid sequence encoding the protein sequence of SEQ ID NO: 53.

In some embodiments, the IL2Rβ-CISC comprises an amino acid sequence as set forth in SEQ ID NO: 55. Embodiments also comprise a nucleic acid sequence encoding the protein sequence of SEQ ID NO: 55.

In some embodiments, the second CISC component is an IL7Ra complex. In some embodiments, the IL7Rα-CISC comprises an amino acid sequence as set forth in SEQ ID NO: 56. Embodiments also comprise a nucleic acid sequence encoding the protein sequence of SEQ ID NO: 56.

In some embodiments, the protein sequence for the second CISC component includes a protein sequence encoding an extracellular binding domain, a hinge domain, a transmembrane domain, or a signaling domain. Embodiments also comprise a nucleic acid sequence encoding the extracellular binding domain, the hinge domain, the transmembrane domain, or the signaling domain of the second CISC component. In some embodiments, the protein sequence of the second CISC component, comprising the second extracellular binding domain, the hinge domain, the transmembrane domain, and/or the signaling domain comprises an amino acid sequence that comprises a 100%, 99%, 98%, 95%, 90%, 85%, or 80% sequence identity to the sequence set forth in SEQ ID NOs: 49, 51, 53, 55, or 56, or has a sequence identity that is within a range defined by any two of the aforementioned percentages.

In some embodiments, the protein sequence may include a linker. In some embodiments, the linker comprises 1, 2, 3, 4, 5, 6, 7, 8, 9 or 10 amino acids, such as glycines, or a number of amino acids, such as glycine, within a range defined by any two of the aforementioned numbers. In some embodiments, the glycine spacer comprises at least 3 glycines. In some embodiments, the glycine spacer comprises a sequence set forth in SEQ ID NO: 62, SEQ ID NO: 63 or SEQ ID NO: 64. Embodiments also comprise a nucleic acid sequence encoding SEQ ID NOs: 62-64. In some embodiments, the transmembrane domain is located N-terminal to the signaling domain, the hinge domain is located N-terminal to the transmembrane domain, the linker is located N-terminal to the hinge domain, and the extracellular binding domain is located N-terminal to the linker.

In some embodiments, a protein sequence or sequences for homodimeric two component CISC are provided. In some embodiments, the first CISC component is an IL2Rγ-CISC complex. In some embodiments, the IL2Rγ-CISC comprises an amino acid sequence as set forth in SEQ ID NO: 58. Embodiments also comprise a nucleic acid sequence encoding the protein sequence of SEQ ID NO: 58.

In some embodiments, the protein sequence for the first CISC component includes a protein sequence encoding an extracellular binding domain, a hinge domain, a transmembrane domain, or a signaling domain. Embodiments also comprise a nucleic acid sequence encoding the extracellular binding domain, the hinge domain, the transmembrane domain, or the signaling domain. In some embodiments, the protein sequence of the first CISC component, comprising the first extracellular binding domain, the hinge domain, the transmembrane domain, and/or the signaling domain comprises an amino acid sequence that comprises a 100%, 99%, 98%, 95%, 90%, 85%, or 80% sequence identity to the sequence set forth in SEQ ID NOs: 58 or has a sequence identity that is within a range defined by any two of the aforementioned percentages.

In some embodiments, the second CISC component is an IL2Rβ complex or an IL2Ra complex. In some embodiments, the IL2Rβ-CISC comprises an amino acid sequence as set forth in SEQ ID NO: 57. Embodiments also comprise a nucleic acid sequence encoding the protein sequence of SEQ ID NO: 57.

In some embodiments, the IL2Rα-CISC comprises an amino acid sequence as set forth in SEQ ID NO: 59. Embodiments also comprise a nucleic acid sequence encoding the protein sequence of SEQ ID NO: 59.

In some embodiments, the protein sequence for the second CISC component includes a protein sequence encoding an extracellular binding domain, a hinge domain, a transmembrane domain, or a signaling domain. Embodiments also comprise a nucleic acid sequence encoding the extracellular binding domain, the hinge domain, the transmembrane domain, or the signaling domain of the second CISC component. In some embodiments, the protein sequence of the second CISC component, comprising the second extracellular binding domain, the hinge domain, the transmembrane domain, and/or the signaling domain comprises an amino acid sequence that comprises a 100%, 99%, 98%, 95%, 90%, 85%, or 80% sequence identity to the sequence set forth in SEQ ID NO: 57 or SEQ ID NO: 59, or has a sequence identity that is within a range defined by any two of the aforementioned percentages.

In some embodiments, the protein sequence may include a linker. In some alternatives, the linker comprises 1, 2, 3, 4, 5, 6, 7, 8, 9 or 10 amino acids, such as glycines, or a number of amino acids, such as glycine, within a range defined by any two of the aforementioned numbers. In some embodiments, the glycine spacer comprises at least 3 glycines. In some embodiments, the glycine spacer comprises a sequence set forth in SEQ ID NO: 62, SEQ ID NO: 63 or SEQ ID NO: 64. Embodiments also comprise a nucleic acid sequence encoding SEQ ID NOs: 62-64. In some embodiments, the transmembrane domain is located N-terminal to the signaling domain, the hinge domain is located N-terminal to the transmembrane domain, the linker is located N-terminal to the hinge domain, and the extracellular binding domain is located N-terminal to the linker.

In some embodiments, the sequences for the homodimerizing two component CISC incorporate FKBP F36V domain for homodimerization with the ligand AP1903.

In some embodiments is provided a protein sequence or sequences for single component homodimerization CISC. In some embodiments, the single component CISC is an IL7Rα-CISC complex. In some embodiments, the IL7Rα-CISC comprises an amino acid sequence as set forth in SEQ ID NO: 60. Embodiments also comprise a nucleic acid sequence encoding the protein sequence of SEQ ID NO: 60.

In some embodiments, the single component CISC is an MPL-CISC complex. In some embodiments, the MPL-CISC comprises an amino acid sequence as set forth in SEQ ID NO: 61. Embodiments also comprise a nucleic acid sequence encoding the protein sequence of SEQ ID NO: 61.

In some embodiments, the protein sequence for the single component CISC includes a protein sequence encoding an extracellular binding domain, a hinge domain, a transmembrane domain, or a signaling domain. Embodiments also comprise a nucleic acid sequence encoding the extracellular binding domain, the hinge domain, the transmembrane domain, or the signaling domain. In some embodiments, the protein sequence of the first CISC component, comprising the first extracellular binding domain, the hinge domain, the transmembrane domain, and/or the signaling domain comprises an amino acid sequence that comprises a 100%, 99%, 98%, 95%, 90%, 85%, or 80% sequence identity to the sequence set forth in SEQ ID NO: 60 or SEQ ID NO: 61 or has a sequence identity that is within a range defined by any two of the aforementioned percentages.

In some embodiments, the protein sequence may include a linker. In some embodiments, the linker comprises 1, 2, 3, 4, 5, 6, 7, 8, 9 or 10 amino acids, such as glycines, or a number of amino acids, such as glycine, within a range defined by any two of the aforementioned numbers. In some embodiments, the glycine spacer comprises at least 3 glycines. In some embodiments, the glycine spacer comprises a sequence set forth in SEQ ID NO: 62, SEQ ID NO: 63 or SEQ ID NO: 64. Embodiments also comprise a nucleic acid sequence encoding SEQ ID NOs: 62-64. In some embodiments, the transmembrane domain is located N-terminal to the signaling domain, the hinge domain is located N-terminal to the transmembrane domain, the linker is located N-terminal to the hinge domain, and the extracellular binding domain is located N-terminal to the linker.

In some embodiments, the sequences for the homodimerizing single component CISC incorporate FKBP F36V domain for homodimerization with the ligand AP1903.

### Methods of Editing Genome

One approach to express a FOXP3 protein or functional derivative thereof in an organism in need thereof is to use genome editing to target the integration of a nucleic acid comprising a coding sequence encoding the FOXP3 protein into an endogenous FOXP3 gene or a non-FOXP3 gene that is sufficiently expressed in a relevant cell type (e.g., T cell) in such a way that expression of the integrated coding sequence is driven by the endogenous promoter of the endogenous FOXP3 gene or non-FOXP3 gene. In some embodiments, where a non-FOXP3 gene is targeted, it is desirable that the expression of the non-FOXP3 gene be specific to the targeted cell type, e.g., lymphocytic cells, e.g., CD4+ cells such as T cells, or cells derived therefrom (e.g., T_{reg} cells) to avoid expression in non-relevant cell types.

In some embodiments, a knock-in strategy involves knocking-in a sequence encoding a FOXP3 or a functional derivative thereof, such as a wild-type FOXP3 gene (e.g., a wild-type human FOXP3 gene), a FOXP3 cDNA, or a FOXP3 minigene (having natural or synthetic enhancer and promoter, one or more exons, and natural or synthetic introns, and natural or synthetic 3′UTR and polyadenylation signal) into a genomic sequence. In some embodiments, the genomic sequence where the FOXP3-encoding sequence is inserted is at, within, or near the FOXP3 locus. In some embodiments, the genomic sequence where the FOXP3-encoding sequence is inserted is at, within, or near exon 1 of the FOXP3 locus.

In some embodiments, provided herein are methods to knock-in a sequence encoding a FOXP3 or a functional derivative thereof into a genome. In one aspect, the present disclosure provides insertion of a nucleic acid comprising a sequence encoding a FOXP3 or a functional derivative thereof into a genome of a cell. In some embodiments, the FOXP3-encoding sequence encodes a wild-type FOXP3. The functional derivative of a FOXP3 can include a derivative of the FOXP3 that has a substantial activity of a wild-type FOXP3, such as a wild-type human FOXP3, e.g., at least at or about 30%, about 40%, about 50%, about 60%, about 70%, about 80%, about 90%, about 95% or about 100% of the activity that the wild-type FOXP3 exhibits. In some embodiments, the functional derivative of a FOXP3 has at least about 30%, about 40%, about 50%, about 60%, about 70%, about 80%, about 85%, about 90%, about 95%, about 96%, about 97%, about 98% or about 99% amino acid sequence identity to FOXP3, e.g., the wild-type FOXP3. In some embodiments, the FOXP3 is encoded by a nucleotide sequence that lacks introns (e.g., a FOXP3 cDNA). One having ordinary skill in the art can use methods known in the art to test the functionality or activity of a FOXP derivative. The functional derivative of a FOXP3 can also include any fragment of the wild-type FOXP3 that has conservative modifications on one or more amino acid residues in the full length, wild-type FOXP3. Thus, in some embodiments, a nucleic acid sequence encoding a functional derivative of a FOXP3 can have at least about 30%, about 40%, about 50%, about 60%, about 70%, about 80%, about 85%, about 90%, about 95%, about 96%, about 97%, about 98% or about 99% nucleic acid sequence identity to a nucleic acid sequence encoding the FOXP3, e.g., the wild-type FOXP3. In some embodiments, the FOXP3 or a functional variant thereof is a human wild-type FOXP3.

In some embodiments, the genome editing methods utilize a DNA endonuclease such as a CRISPR/Cas endonuclease to genetically introduce (knock-in) a sequence encoding a FOXP3 or a functional derivative thereof. In some embodiments, the DNA endonuclease is a Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, or Cpf1 endonuclease, a homolog thereof, a recombinant of the naturally occurring molecule, a codon-optimized, or modified version thereof, or a combination of any of the foregoing. In some embodiments, the DNA endonuclease is a Cas9. In some embodiments, the Cas9 is from Streptococcus pyogenes (spCas9). In some embodiments, the Cas9 is from Staphylococcus lugdunensis (SluCas9).

In some embodiments, the cell subject to the genome-edition has one or more mutation(s) in the genome which results in a decrease of the expression of an endogenous FOXP3 gene as compared to the expression in a normal cell that does not have such mutation(s). The normal cell can be a healthy or control cell that is originated (or isolated) from a different subject who does not have FOXP3 gene defects. In some embodiments, the cell subject to the genome-edition can be originated (or isolated) from a subject who is in need of treatment of a FOXP3 gene related condition or disorder, e.g. a subject suffering from an autoimmune disorder (e.g., IPEX syndrome). Therefore, in some embodiments the expression of an endogenous FOXP3 gene in such cell is about 10%, about 20%, about 30%, about 40%, about 50%, about 60%, about 70%, about 80%, about 90% or about 100% decreased as compared to the expression of an endogenous FOXP3 gene in the normal cell.

In some embodiments, provided herein is a method of editing a genome in a lymphocytic cell, the method comprising providing the following to the lymphocytic cell: (a) a Cas DNA endonuclease (e.g., a Cas9 endonuclease) or nucleic acid encoding the Cas DNA endonuclease; (b) a gRNA (e.g., an sgRNA) or nucleic acid encoding the gRNA, wherein the gRNA is capable of targeting the Cas DNA endonuclease to a FOXP3 locus or a non-FOXP3 locus (e.g., AAVS1) in the genome of a cell, and (c) a donor template comprising a FOXP3 coding sequence. In some embodiments, the Cas DNA endonuclease is a Cas9 endonuclease (e.g., a Cas9 endonuclease from Streptococcus pyogenes). In some embodiments, the gRNA comprises a spacer sequence complementary to a target sequence in a FOXP3 locus. In some embodiments, the gRNA comprises a spacer sequence complementary to a target sequence in exon 1 of a FOXP3 locus. In some embodiments, the gRNA comprises a spacer sequence from any one of SEQ ID NOs: 1-7 and 27-29 or a variant thereof having no more than 3 mismatches compared to any one of SEQ ID NOs: 1-7 and 27-29. In some embodiments, the gRNA comprises a spacer sequence from any one of SEQ ID NOs: 1-7 or a variant thereof having no more than 3 mismatches compared to any one of SEQ ID NOs: 1-7. In some embodiments, the gRNA comprises a spacer sequence from any one of SEQ ID NOs: 2, 3, and 5, or a variant thereof having no more than 3 mismatches compared to any one of SEQ ID NOs: 2, 3, and 5. In some embodiments, the gRNA comprises a spacer sequence complementary to a target sequence in a non-FOXP3 locus (e.g., AAVS1). In some embodiments, the gRNA comprises a spacer sequence from any one of SEQ ID NOs: 15-20 or a variant thereof having no more than 3 mismatches compared to any one of SEQ ID NOs: 15-20. In some embodiments, the FOXP3 coding sequence encodes FOXP3 or a functional derivative thereof. In some embodiments, the FOXP3 coding sequence is a FOXP3 cDNA. An exemplary FOXP3 cDNA sequence can be found in the AAV donor template having the nucleotide sequence of SEQ ID NO: 34. In some embodiments, the method comprises providing to the lymphocytic cell the Cas DNA endonuclease. In some embodiments, the method comprises providing to the lymphocytic cell nucleic acid encoding the Cas DNA endonuclease. In some embodiments, the method comprises providing to the lymphocytic cell the gRNA. In some embodiments, the gRNA is an sgRNA. In some embodiments, the method comprises providing to the lymphocytic cell nucleic acid encoding the gRNA. In some embodiments, the method further comprises providing to the lymphocytic cell one or more additional gRNAs or nucleic acid encoding the one or more additional gRNAs.

In some embodiments, according to any of the methods of editing a genome in a cell described herein, the DNA endonuclease is a Cas9. In some embodiments, the Cas9 is from Streptococcus pyogenes (spCas9). In some embodiments, the Cas9 is from Staphylococcus lugdunensis (SluCas9).

In some embodiments, according to any of the methods of editing a genome in a cell described herein, the nucleic acid sequence encoding a FOXP3 or a functional derivative thereof is codon-optimized for expression in the cell. In some embodiments, the nucleic acid sequence encoding a FOXP3 or a functional derivative thereof has at least about 70% sequence identity, e.g., at least about 75%, 80%, 85%, 90%, 92%, 93%, 94%, 95%, 96%, 97%, 98%, 99% or greater sequence identity, to a sequence according to SEQ ID NO: 68. In some embodiments, the cell is a human cell.

In some embodiments, according to any of the methods of editing a genome in a cell described herein, the method employs a nucleic acid encoding the DNA endonuclease. In some embodiments, the nucleic acid encoding the DNA endonuclease is codon-optimized for expression in the cell. In some embodiments, the cell is a human cell, e.g., a human CD4+ T cell. In some embodiments, the nucleic acid encoding the DNA endonuclease is DNA, such as a DNA plasmid. In some embodiments, the nucleic acid encoding the DNA endonuclease is RNA, such as mRNA.

In some embodiments, according to any of the methods of editing a genome in a cell described herein, the donor template comprises a donor cassette comprising the nucleic acid sequence encoding a FOXP3 or a functional derivative thereof, and the donor template is configured such that the donor cassette is capable of being integrated into the genomic locus targeted by the gRNA of (b) by homology directed repair (HDR). In some embodiments, the donor cassette is flanked on both sides by homology arms corresponding to sequences in the targeted genomic locus. In some embodiments, the homology arms are at least about 0.2 kb (such as at least about any of 0.3 kb, 0.4 kb, 0.5 kb, 0.6 kb, 0.7 kb, 0.8 kb, 0.9 kb, 1 kb, or greater) in length. In some embodiments, the homology arms are at least about 0.4 kb in length. Exemplary homology arms include 5′-homology arms having the sequence of any one of SEQ ID NOs: 90-97 and 106-107, and 3′-homology arms having the sequence of any one of SEQ ID NOs: 98-105 and 108-109. In some embodiments, the homology arms at the 5′- and 3′-ends of the donor template are the same. In some embodiments, the homology arms at the 5′- and 3′-ends of the donor template are different.

In some embodiments, the donor template is encoded in an Adeno Associated Virus (AAV) vector. In some embodiments, the AAV vector is an AAV6 vector.

In some embodiments, according to any of the methods of editing a genome in a cell described herein, the donor template comprises a donor cassette comprising the nucleic acid sequence encoding a FOXP3 or a functional derivative thereof, and the donor template is configured such that the donor cassette is capable of being integrated into the genomic locus targeted by the gRNA of (b) by non-homologous end joining (NE-IEJ). In some embodiments, the donor cassette is flanked on one or both sides by a gRNA target site. In some embodiments, the donor cassette is flanked on both sides by a gRNA target site. In some embodiments, the gRNA target site is a target site for a gRNA in the system. In some embodiments, the gRNA target site of the donor template is the reverse complement of a cell genome gRNA target site for a gRNA in the system. In some embodiments, the donor template is encoded in an Adeno Associated Virus (AAV) vector. In some embodiments, the AAV vector is an AAV6 vector.

In some embodiments, according to any of the methods of editing a genome in a cell described herein, the DNA endonuclease or nucleic acid encoding the DNA endonuclease is formulated in a liposome or lipid nanoparticle. In some embodiments, the liposome or lipid nanoparticle also comprises the gRNA. In some embodiments, the liposome or lipid nanoparticle is a lipid nanoparticle. In some embodiments, the method employs a lipid nanoparticle comprising nucleic acid encoding the DNA endonuclease and the gRNA. In some embodiments, the nucleic acid encoding the DNA endonuclease is an mRNA encoding the DNA endonuclease.

In some embodiments, according to any of the methods of editing a genome in a cell described herein, the DNA endonuclease is pre-complexed with the gRNA, forming a ribonucleoprotein (RNP) complex. In some embodiments, the RNP complex is provided to the cell by electroporation. In some embodiments, the donor template is an AAV donor template encoded in an AAV vector (e.g., an AAV6 vector). In some embodiments, the AAV donor template is provided to the cell at or around the same time that the RNP complex is provided to the cell. For example, in some embodiments, the cell is electroporated with the RNP complex and transduced with the AAV donor template on the same day. In some embodiments, the cell is electroporated with the RNP complex and transduced with the AAV donor template, wherein the electroporation and transduction are carried out no greater than about 12 hours (such as no greater than about any of 11 hours, 10 hours, 9 hours, 8 hours, 7 hours, 6 hours, 5 hours, 4 hours, 3 hours, 2 hours, 1 hour, or less) apart. In some embodiments, the cell is electroporated with the RNP complex, plated, and transduced with the AAV donor template. In some embodiments, the cell is pre-stimulated in the presence of factors capable of activating and expanding the cell (e.g., anti-CD3 and/or anti-CD28 antibodies, such as anti-CD3/anti-CD28 beads) prior to providing the RNP and AAV donor template to the cell. In some embodiments, the pre-stimulation is carried out for at least about 12 hours (such as at least about any of 16 hours, 20 hours, 24 hours, 36 hours, 48 hours, 60 hours, 72 hours, or more). In some embodiments, the pre-stimulation is carried out for at least about 72 hours. In some embodiments, the pre-stimulation is carried out in a cell composition comprising between about 1×10⁵ and 1×10⁷ (such about any of 2.5×10⁵, 5×10⁵, 7.5×10⁵, 1×10⁶, 2.5×10⁶, 5×10⁶, and 7.5×10⁶, including any ranges between these values) cells/ml. In some embodiments, the concentration of cells in the cell composition is about 5×10⁵ cells/ml.

In some embodiments, according to any of the methods of editing a genome in a cell described herein, the frequency of targeted integration of the donor template into a FOXP3 locus in the cell genome is from about 0.1% to about 99%. In some embodiments, the frequency of targeted integration is from about 2% to about 70% (such as from about 2% to about 65%, from about 2% to about 55%, from about 3% to about 70%, from about 5% to about 70%, from about 5% to about 60%, from about 5% to about 50%, or from about 10% to about 50%). In some embodiments, the cell is a cell in a subject, such as a human subject.

In some embodiments, according to any of the methods of editing a genome in a cell described herein, the cell is cryopreserved following editing.

### Target Sequence Selection

In some embodiments, shifts in the location of the 5′ boundary and/or the 3′ boundary relative to particular reference loci are used to facilitate or enhance particular applications of gene editing, which depend in part on the endonuclease system selected for the editing, as further described and illustrated herein.

In a first, non-limiting aspect of such target sequence selection, many endonuclease systems have rules or criteria that guide the initial selection of potential target sites for cleavage, such as the requirement of a PAM sequence motif in a particular position adjacent to the DNA cleavage sites in the case of CRISPR Type II or Type V endonucleases.

In another, non-limiting aspect of target sequence selection or optimization, the frequency of “off-target” activity for a particular combination of target sequence and gene editing endonuclease (such as the frequency of DSBs occurring at sites other than the selected target sequence) is assessed relative to the frequency of on-target activity. In some cases, cells that have been correctly edited at the desired locus can have a selective advantage relative to other cells. Illustrative, but non-limiting, examples of a selective advantage include the acquisition of attributes such as enhanced rates of replication, persistence, resistance to certain conditions, enhanced rates of successful engraftment or persistence in vivo following introduction into a subject, and other attributes associated with the maintenance or increased numbers or viability of such cells. In other cases, cells that have been correctly edited at the desired locus can be positively selected for by one or more screening methods used to identify, sort, or otherwise select for cells that have been correctly edited. Both selective advantage and directed selection methods can take advantage of the phenotype associated with the correction. In some embodiments, cells can be edited two or more times to create a second modification that creates a new phenotype that is used to select or purify the intended population of cells. Such a second modification could be created by adding a second gRNA for a selectable or screenable marker. In some cases, cells can be correctly edited at the desired locus using a DNA fragment that contains the cDNA and also a selectable marker.

In embodiments, whether any selective advantage is applicable or any directed selection is to be applied in a particular case, target sequence selection is also guided by consideration of off-target frequencies to enhance the effectiveness of the application and/or reduce the potential for undesired alterations at sites other than the desired target. As described further and illustrated herein and in the art, the occurrence of off-target activity is influenced by a number of factors including similarities and dissimilarities between the target site and various off-target sites, as well as the particular endonuclease used. Bioinformatics tools are available that assist in the prediction of off-target activity, and frequently such tools can also be used to identify the most likely sites of off-target activity, which can then be assessed in experimental settings to evaluate relative frequencies of off-target to on-target activity, thereby allowing the selection of sequences that have higher relative on-target activities. Illustrative examples of such techniques are provided herein, and others are known in the art.

Another aspect of target sequence selection relates to homologous recombination events. Sequences sharing regions of homology can serve as focal points for homologous recombination events that result in deletion of intervening sequences. Such recombination events occur during the normal course of replication of chromosomes and other DNA sequences, and also at other times when DNA sequences are being synthesized, such as in the case of repairs of double-strand breaks (DSBs), which occur on a regular basis during the normal cell replication cycle but can also be enhanced by the occurrence of various events (such as UV light and other inducers of DNA breakage) or the presence of certain agents (such as various chemical inducers). Many such inducers cause DSBs to occur indiscriminately in the genome, and DSBs are regularly being induced and repaired in normal cells. During repair, the original sequence can be reconstructed with complete fidelity, however, in some cases, small insertions or deletions (referred to as “indels”) are introduced at the DSB site.

DSBs can also be specifically induced at particular locations, as in the case of the endonucleases systems described herein, which can be used to cause directed or preferential gene modification events at selected chromosomal locations. The tendency for homologous sequences to be subject to recombination in the context of DNA repair (as well as replication) can be taken advantage of in a number of circumstances, and is the basis for one application of gene editing systems, such as CRISPR, in which homology directed repair is used to insert a sequence of interest, provided through use of a “donor” polynucleotide, into a desired chromosomal location.

Regions of homology between particular sequences, which can be small regions of “microhomology” that can have as few as ten base pairs or less, can also be used to bring about desired deletions. For example, a single DSB is introduced at a site that exhibits microhomology with a nearby sequence. During the normal course of repair of such DSB, a result that occurs with high frequency is the deletion of the intervening sequence as a result of recombination being facilitated by the DSB and concomitant cellular repair process.

In some circumstances, however, selecting target sequences within regions of homology can also give rise to much larger deletions, including gene fusions (when the deletions are in coding regions), which can or cannot be desired given the particular circumstances.

The examples provided herein further illustrate the selection of various target regions for the creation of DSBs designed to insert a FOXP3-encoding gene, as well as the selection of specific target sequences within such regions that are designed to minimize off-target events relative to on-target events. In some embodiments, the target locus is selected from a FOXP3 locus, an AAVS1 locus, and a TCRa (TRAC) locus.

### Nucleic Acid Modifications

In some embodiments, polynucleotides introduced into cells have one or more modifications that can be used individually or in combination, for example, to enhance activity, stability, or specificity, alter delivery, reduce innate immune responses in host cells, or for other enhancements, as further described herein and known in the art.

In certain embodiments, modified polynucleotides are used in the CRISPR/Cas9 system, in which case the guide RNAs (either single-molecule guides or double-molecule guides) and/or a DNA or an RNA encoding a Cas endonuclease introduced into a cell can be modified, as described and illustrated below. Such modified polynucleotides can be used in the CRISPR/Cas9 system to edit any one or more genomic loci.

Using the CRISPR/Cas9 system for purposes of non-limiting illustrations of such uses, modifications of guide RNAs can be used to enhance the formation or stability of the CRISPR/Cas9 genome editing complex having guide RNAs, which can be single-molecule guides or double-molecule, and a Cas endonuclease. Modifications of guide RNAs can also or alternatively be used to enhance the initiation, stability, or kinetics of interactions between the genome editing complex with the target sequence in the genome, which can be used, for example, to enhance on-target activity. Modifications of guide RNAs can also or alternatively be used to enhance specificity, e.g., the relative rates of genome editing at the on-target site as compared to effects at other (off-target) sites.

Modifications can also or alternatively be used to increase the stability of a guide RNA, e.g., by increasing its resistance to degradation by ribonucleases (RNases) present in a cell, thereby causing its half-life in the cell to be increased. Modifications enhancing guide RNA half-life can be particularly useful in embodiments in which a Cas endonuclease is introduced into the cell to be edited via an RNA that needs to be translated to generate endonuclease, because increasing the half-life of guide RNAs introduced at the same time as the RNA encoding the endonuclease can be used to increase the time that the guide RNAs and the encoded Cas or Cpf1 endonuclease co-exist in the cell.

Modifications can also or alternatively be used to decrease the likelihood or degree to which RNAs introduced into cells elicit innate immune responses. Such responses, which have been well characterized in the context of RNA interference (RNAi), including small-interfering RNAs (siRNAs), as described below and in the art, tend to be associated with reduced half-life of the RNA and/or the elicitation of cytokines or other factors associated with immune responses.

One or more types of modifications can also be made to RNAs encoding an endonuclease that are introduced into a cell, including, without limitation, modifications that enhance the stability of the RNA (such as by increasing its degradation by RNAses present in the cell), modifications that enhance translation of the resulting product (such as the endonuclease), and/or modifications that decrease the likelihood or degree to which the RNAs introduced into cells elicit innate immune responses.

Combinations of modifications, such as the foregoing and others, can likewise be used. In the case of CRISPR/Cas9, for example, one or more types of modifications can be made to guide RNAs (including those exemplified above), and/or one or more types of modifications can be made to RNAs encoding Cas endonuclease (including those exemplified above).

### Delivery

In some embodiments, any nucleic acid molecules used in the methods provided herein, e.g., a nucleic acid encoding a genome-targeting nucleic acid of the disclosure and/or a site-directed polypeptide, are packaged into or on the surface of delivery vehicles for delivery to cells. Delivery vehicles contemplated include, but are not limited to, nanospheres, liposomes, quantum dots, nanoparticles, polyethylene glycol particles, hydrogels, and micelles. As described in the art, a variety of targeting moieties can be used to enhance the preferential interaction of such vehicles with desired cell types or locations.

Introduction of the complexes, polypeptides, and nucleic acids of the disclosure into cells can occur by viral or bacteriophage infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, calcium phosphate precipitation, direct micro-injection, nanoparticle-mediated nucleic acid delivery, and the like.

In embodiments, guide RNA polynucleotides (RNA or DNA) and/or endonuclease polynucleotide(s) (RNA or DNA) can be delivered by viral or non-viral delivery vehicles known in the art. Alternatively, endonuclease polypeptide(s) can be delivered by viral or non-viral delivery vehicles known in the art, such as electroporation or lipid nanoparticles. In some embodiments, the DNA endonuclease can be delivered as one or more polypeptides, either alone or pre-complexed with one or more guide RNAs, or one or more crRNA together with a tracrRNA.

In embodiments, polynucleotides can be delivered by non-viral delivery vehicles including, but not limited to, nanoparticles, liposomes, ribonucleoproteins, positively charged peptides, small molecule RNA-conjugates, aptamer-RNA chimeras, and RNA-fusion protein complexes. Some exemplary non-viral delivery vehicles are described in Peer, D. et al. (2011). Gene Therapy, 18:1127-1133 (which focuses on non-viral delivery vehicles for siRNA that are also useful for delivery of other polynucleotides).

In embodiments, polynucleotides, such as guide RNA, sgRNA, and mRNA encoding an endonuclease, can be delivered to a cell or a subject by a lipid nanoparticle (LNP).

While several non-viral delivery methods for nucleic acids have been tested both in animal models and in humans the most well developed system is lipid nanoparticles. Lipid nanoparticles (LNP) are generally composed of an ionizable cationic lipid and 3 or more additional components, generally cholesterol, DOPE, and a polyethylene glycol (PEG) containing lipid, see, e.g. Example 2. The cationic lipid can bind to the positively charged nucleic acid forming a dense complex that protects the nucleic from degradation. During passage through a micro fluidics system the components self-assemble to form particles in the size range of 50 to 150 nM in which the nucleic acid is encapsulated in the core complexed with the cationic lipid and surrounded by a lipid bilayer like structure. After injection into the circulation of a subject these particles can bind to apolipoprotein E (apoE). ApoE is a ligand for the LDL receptor and mediates uptake into the hepatocytes of the liver via receptor mediated endocytosis. LNP of this type have been shown to efficiently deliver mRNA and siRNA to the hepatocytes of the liver of rodents, primates, and humans. After endocytosis, the LNP are present in endosomes. The encapsulated nucleic acid undergoes a process of endosomal escape mediate by the ionizable nature of the cationic lipid. This delivers the nucleic acid into the cytoplasm where mRNA can be translated into the encoded protein. After endosomal escape the Cas9 mRNA is translated into Cas9 protein and can form a complex with the gRNA. In some embodiments, inclusion of a nuclear localization signal into the Cas9 protein sequence promotes translocation of the Cas9 protein/gRNA complex to the nucleus. Alternatively, the small gRNA crosses the nuclear pore complex and form complexes with Cas9 protein in the nucleus. Once in the nucleus the gRNA/Cas9 complex scan the genome for homologous target sites and generate double-strand breaks preferentially at the desired target site in the genome. The half-life of RNA molecules in vivo is generally short, on the order of hours to days. Similarly, the half-life of proteins tends to be short, on the order of hours to days. Thus, in some embodiments, delivery of the gRNA and Cas9 mRNA using an LNP can result in only transient expression and activity of the gRNA/Cas9 complex. This can provide the advantage of reducing the frequency of off-target cleavage and thus minimize the risk of genotoxicity in some embodiments. LNP are generally less immunogenic than viral particles. While many humans have preexisting immunity to AAV there is no pre-existing immunity to LNP. In additional and adaptive immune response against LNP is unlikely to occur which enables repeat dosing of LNP.

Several different ionizable cationic lipids have been developed for use in LNP. These include C12-200 (Love, K. T. et al. (2010). Proc. Natl. Acad. Sci. U.S.A., 107(5):1864-1869), MC3, LN16, MD1 among others. In one type of LNP a GalNac moiety is attached to the outside of the LNP and acts as a ligand for uptake into the liver via the asialyloglycoprotein receptor. Any of these cationic lipids are used to formulate LNP for delivery of gRNA and Cas9 mRNA to the liver.

In some embodiments, an LNP refers to any particle having a diameter of less than 1000 nm, 500 nm, 250 nm, 200 nm, 150 nm, 100 nm, 75 nm, 50 nm, or 25 nm. Alternatively, a nanoparticle can range in size from 1-1000 nm, 1-500 nm, 1-250 nm, 25-200 nm, 25-100 nm, 35-75 nm, or 25-60 nm.

LNPs can be made from cationic, anionic, or neutral lipids. Neutral lipids, such as the fusogenic phospholipid DOPE or the membrane component cholesterol, can be included in LNPs as ‘helper lipids’ to enhance transfection activity and nanoparticle stability. Limitations of cationic lipids include low efficacy owing to poor stability and rapid clearance, as well as the generation of inflammatory or anti-inflammatory responses. LNPs can also have hydrophobic lipids, hydrophilic lipids, or both hydrophobic and hydrophilic lipids.

Any lipid or combination of lipids that are known in the art can be used to produce an LNP. Examples of lipids used to produce LNPs are: DOTMA, DOSPA, DOTAP, DMRIE, DC-cholesterol, DOTAP-cholesterol, GAP-DMORIE-DPyPE, and GL67A-DOPE-DMPE-polyethylene glycol (PEG). Examples of cationic lipids are: 98N12-5, C12-200, DLin-KC2-DMA (KC2), DLin-MC3-DMA (MC3), XTC, MD1, and 7C1. Examples of neutral lipids are: DPSC, DPPC, POPC, DOPE, and SM. Examples of PEG-modified lipids are: PEG-DMG, PEG-CerC14, and PEG-CerC20.

In embodiments, the lipids can be combined in any number of molar ratios to produce an LNP. In addition, the polynucleotide(s) can be combined with lipid(s) in a wide range of molar ratios to produce an LNP.

In embodiments, the site-directed polypeptide and genome-targeting nucleic acid can each be administered separately to a cell or a subject. On the other hand, the site-directed polypeptide can be pre-complexed with one or more guide RNAs, or one or more crRNA together with a tracrRNA. The pre-complexed material can then be administered to a cell or a subject. Such pre-complexed material is known as a ribonucleoprotein particle (RNP).

RNA can form specific interactions with RNA or DNA. While this property is exploited in many biological processes, it also comes with the risk of promiscuous interactions in a nucleic acid-rich cellular environment. One solution to this problem is the formation of ribonucleoprotein particles (RNPs), in which the RNA is pre-complexed with an endonuclease. Another benefit of the RNP is protection of the RNA from degradation.

In some embodiments, the endonuclease in the RNP can be modified or unmodified. Likewise, the gRNA, crRNA, tracrRNA, or sgRNA can be modified or unmodified. Numerous modifications are known in the art and can be used.

The endonuclease and sgRNA can be generally combined in a 1:1 molar ratio.

Alternatively, the endonuclease, crRNA, and tracrRNA can be generally combined in a 1:1:1 molar ratio. However, a wide range of molar ratios can be used to produce an RNP.

In some embodiments, a recombinant adeno-associated virus (AAV) vector can be used for delivery. Techniques to produce rAAV particles, in which an AAV genome to be packaged that includes the polynucleotide to be delivered, rep, and cap genes, and helper virus functions are provided to a cell are known in the art. Production of rAAV requires that the following components are present within a single cell (denoted herein as a packaging cell): a rAAV genome, AAV rep and cap genes separate from (such as not in) the rAAV genome, and helper virus functions. The AAV rep and cap genes can be from any AAV serotype for which recombinant virus can be derived, and can be from a different AAV serotype than the rAAV genome ITRs, including, but not limited to, AAV serotypes AAV-1, AAV-2, AAV-3, AAV-4, AAV-5, AAV-6, AAV-7, AAV-8, AAV-9, AAV-10, AAV-11, AAV-12, AAV-13, and AAV rh.74. Production of pseudotyped rAAV is disclosed in, for example, International Patent Application no. WO 01/83692. See Table 1. Table 1 shows AAV serotype and Genbank Accession No. of some selected AAVs.

TABLE 1

| AAV Serotype | Genbank Accession No. |
|---|---|
| AAV-1 | NC_002077.1 |
| AAV-2 | NC_001401.2 |
| AAV-3 | NC_001729.1 |
| AAV-3B | AF028705.1 |
| AAV-4 | NC_001829.1 |
| AAV-5 | NC_006152.1 |
| AAV-6 | AF028704.1 |
| AAV-7 | NC_006260.1 |
| AAV-8 | NC_006261.1 |
| AAV-9 | AX753250.1 |
| AAV-10 | AY631965.1 |
| AAV-11 | AY631966.1 |
| AAV-12 | DQ813647.1 |
| AAV-13 | EU285562.1 |

In some embodiments, a method of generating a packaging cell involves creating a cell line that stably expresses all of the necessary components for AAV particle production. For example, a plasmid (or multiple plasmids) having a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing (Samulski, R. J. et al. (1982). Proc. Natl. Acad. Sci. U.S.A., 79(6):2077-2081), addition of synthetic linkers containing restriction endonuclease cleavage sites (Laughlin, C. A. et al. (1983). Gene, 23(1):65-73) or by direct, blunt-end ligation (Senapathy, P. et al. (1984). J. Biol. Chem., 259:4661-4666). The packaging cell line is then infected with a helper virus, such as adenovirus. The advantages of this method are that the cells are selectable and are suitable for large-scale production of rAAV. Other examples of suitable methods employ adenovirus or baculovirus, rather than plasmids, to introduce rAAV genomes and/or rep and cap genes into packaging cells.

General principles of rAAV production are reviewed in, for example, Carter, B. J. (1992). Curr. Opin. Biotechnol., 3(5): 533-539; and Muzyczka, M. (1992). Curr. Top. Microbiol. Immunol., 158:97-129). Various approaches are described in Tratschin, J. D. et al. (1984). Mol. Cell. Biol., 4(10):2072-2081; Hermonat, P. L. et al. (1984). Proc. Natl. Acad. Sci. U.S.A., 81(20):6466-6470; Tratschin, J. D. et al. (1985). Mol. Cell. Biol. 5(11):3251-3260; McLaughlin, S. K. et al. (1988). J. Virol., 62(6):1963-1973; and Lebkowski, J. S. et al. (1988). Mol. Cell. Biol., 8(10):3988-3996. Samulski, R. J. et al. (1989), J. Virol., 63(9):3822-3828; U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin, P. et al. (1995). Vaccine, 13(13):1244-1250; Paul, R. W. et al. (1993). Hum. Gene Ther., 4(5):609-615; Clark, K. R. et al. (1996). Gene Ther. 3(12):1124-1132; U.S. Pat. Nos. 5,786,211; 5,871,982; and 6,258,595.

AAV vector serotypes can be matched to target cell types. For example, the following exemplary cell types can be transduced by the indicated AAV serotypes among others. For instance, the serotypes of AAV vectors suitable to hematopoietic stem cell include, but not limited to, AAV2 and AAV6. In some embodiments, the AAV vector serotype is AAV6.

In some embodiments, the AAV vector comprises a nucleic acid sequence having at least at or about 90% sequence identity (e.g., at least 92%, 93%, 94%, 95%, 96%, 97%, 98%, 99%, 99.2%, 99.5%, 99.6%, 99.7%, 99.8%, 99.9%, or greater) to any one of SEQ ID NOs: 33-36 and 161. In some embodiments, the AAV vector comprises a nucleic acid sequence having at least at or about 90% sequence identity (e.g., at least 92%, 93%, 94%, 95%, 96%, 97%, 98%, 99%, 99.2%, 99.5%, 99.6%, 99.7%, 99.8%, 99.9%, or greater) to SEQ ID NO: 33. In some embodiments, the AAV vector comprises a nucleic acid sequence having at least at or about 90% sequence identity (e.g., at least 92%, 93%, 94%, 95%, 96%, 97%, 98%, 99%, 99.2%, 99.5%, 99.6%, 99.7%, 99.8%, 99.9%, or greater) to SEQ ID NO: 34. In some embodiments, the AAV vector comprises a nucleic acid sequence having at least at or about 90% sequence identity (e.g., at least 92%, 93%, 94%, 95%, 96%, 97%, 98%, 99%, 99.2%, 99.5%, 99.6%, 99.7%, 99.8%, 99.9%, or greater) to SEQ ID NO: 35. In some embodiments, the AAV vector comprises a nucleic acid sequence having at least at or about 90% sequence identity (e.g., at least 92%, 93%, 94%, 95%, 96%, 97%, 98%, 99%, 99.2%, 99.5%, 99.6%, 99.7%, 99.8%, 99.9%, or greater) to SEQ ID NO: 36. In some embodiments, the AAV vector comprises a nucleic acid sequence having at least at or about 90% sequence identity (e.g., at least 92%, 93%, 94%, 95%, 96%, 97%, 98%, 99%, 99.2%, 99.5%, 99.6%, 99.7%, 99.8%, 99.9%, or greater) to SEQ ID NO: 161.

In addition to adeno-associated viral vectors, other viral vectors can be used. Such viral vectors include, but are not limited to, lentivirus, alphavirus, enterovirus, pestivirus, baculovirus, herpesvirus, Epstein Barr virus, papovavirus, poxvirus, vaccinia virus, and herpes simplex virus.

In some embodiments, Cas9 mRNA, sgRNA targeting one or two loci in FOXP3 genes, and donor DNA are each separately formulated into lipid nanoparticles, or are all co-formulated into one lipid nanoparticle, or co-formulated into two or more lipid nanoparticles.

In some embodiments, Cas9 mRNA is formulated in a lipid nanoparticle, while sgRNA and donor DNA are delivered in an AAV vector. In some embodiments, Cas9 mRNA and sgRNA are co-formulated in a lipid nanoparticle, while donor DNA is delivered in an AAV vector.

Options are available to deliver the Cas9 nuclease as a DNA plasmid, as mRNA or as a protein. The guide RNA can be expressed from the same DNA, or can be delivered as an RNA. The RNA can be chemically modified to alter or improve its half-life and/or decrease the likelihood or degree of immune response. The endonuclease protein can be complexed with the gRNA prior to delivery. Viral vectors allow efficient delivery; split versions of Cas9 and smaller orthologs of Cas9 can be packaged in AAV, as can donors for HDR. A range of non-viral delivery methods also exist that can deliver each of these components, or non-viral and viral methods can be employed in tandem. For example, nanoparticles can be used to deliver the protein and guide RNA, while AAV can be used to deliver a donor DNA.

In some embodiments that are related to deliver genome-editing components for therapeutic treatments, at least two components are delivered into the nucleus of a cell to be transformed, e.g., lymphocytic cells; a sequence-specific nuclease and a DNA donor template. In some embodiments, the AAV is selected from the serotypes AAV2 and AAV6. In some embodiments, the AAV packaged DNA donor template is administered to a subject, e.g., a human subject, first by peripheral IV injection followed by the sequence-specific nuclease. The advantage of delivering an AAV packaged donor DNA template first is that the delivered donor DNA template will be stably maintained in the nucleus of the transduced lymphocytic cells which allows for the subsequent administration of the sequence-specific nuclease which will create a double-strand break in the genome with subsequent integration of the DNA donor by HDR or NHEJ. It is desirable in some embodiments that the sequence-specific nuclease remain active in the target cell only for the time required to promote targeted integration of the transgene at sufficient levels for the desired therapeutic effect. If the sequence-specific nuclease remains active in the cell for an extended duration this will result in an increased frequency of double-strand breaks at off-target sites. Specifically, the frequency of off-target cleavage is a function of the off-target cutting efficiency multiplied by the time over which the nuclease is active. Delivery of a sequence-specific nuclease in the form of a mRNA results in a short duration of nuclease activity in the range of hours to a few days because the mRNA and the translated protein are short lived in the cell. Thus, delivery of the sequence-specific nuclease into cells that already contain the donor template is expected to result in the highest possible ratio of targeted integration relative to off-target integration.

In some embodiments, the sequence-specific nuclease is CRISPR-Cas9 which is composed of a sgRNA directed to a FOXP3 locus together with a Cas9 nuclease. In some embodiments, the Cas9 nuclease is delivered as a mRNA encoding the Cas9 protein operably fused to one or more nuclear localization signals (NLS). In some embodiments, the sgRNA and the Cas9 mRNA are delivered to the lymphocytic cell, e.g., a CD4+ T cell, by packaging into a lipid nanoparticle.

In some embodiments, to promote nuclear localization of a donor template, DNA sequence that can promote nuclear localization of plasmids, e.g., a 366 bp region of the simian virus 40 (SV40) origin of replication and early promoter, can be added to the donor template. Other DNA sequences that bind to cellular proteins can also be used to improve nuclear entry of DNA.

### Genetically Modified Cells and Cell Populations

In one aspect, the disclosures herewith provide a method of editing a genome in a cell, thereby creating a genetically modified cell. In some aspects, a population of genetically modified cells are provided. The genetically modified cell therefore refers to a cell that has at least one genetic modification introduced by genome editing (e.g., using the CRISPR/Cas9 system). In some embodiments, the genetically modified cell is a genetically modified lymphocytic cell, e.g. a T cell such as a human CD4+ T cell. In some embodiments, the T cell is a human T cell from an IPEX subject. A genetically modified cell having an integrated FOXP3 coding sequence is contemplated herein.

The compositions described herein provide for genetically modified cells, such as mammalian cells, which include the protein sequences or the expression vectors as set forth and described herein. Accordingly, provided herein are cells, such as mammalian cells, for dimeric CISC secretion, wherein the cell comprises the protein sequences of any one of the embodiments described herein or the expression vector of any one of the embodiments described herein. In some embodiments, the cell is a mammalian cell, such as a lymphocyte. In some embodiments, the cell is a lymphocytic cell, such as a lymphocyte.

In some embodiments, the cells are precursor T cells or T regulatory cells. In some embodiments, the cells stem cells, such as hematopoietic stem cells. In some embodiments, the cell is a NK cell. In some embodiments, the cells are CD34+, CD8+, and/or CD4+T lymphocytes. In some embodiments, the cell is a B cell. In some embodiments, the cell is a neuronal stem cell.

In some embodiments, the cells are CD8+T cytotoxic lymphocyte cells, which may include naïve CD8+ T cells, central memory CD8+ T cells, effector memory CD8+ T cells, or bulk CD8+ T cells. In some embodiments, the cells are CD4+T helper lymphocyte cells, which may include naïve CD4+ T cells, central memory CD4+ T cells, effector memory CD4+ T cells, or bulk CD4+ T cells.

The lymphocytes (T lymphocytes) can be collected in accordance with known techniques and enriched or depleted by known techniques such as affinity binding to antibodies such as flow cytometry and/or immunomagnetic selection. After enrichment and/or depletion steps, in vitro expansion of the desired T lymphocytes can be carried out in accordance with known techniques or variations thereof that will be apparent to those skilled in the art. In some embodiments, the T cells are autologous T cells obtained from a patient.

For example, the desired T cell population or subpopulation can be expanded by adding an initial T lymphocyte population to a culture medium in vitro, and then adding to the culture medium feeder cells, such as non-dividing peripheral blood mononuclear cells (PBMC), (e.g., such that the resulting population of cells contains at least 5, 10, 20, or 40 or more PBMC feeder cells for each T lymphocyte in the initial population to be expanded); and incubating the culture (e.g. for a time sufficient to expand the numbers of T cells). The non-dividing feeder cells can comprise gamma-irradiated PBMC feeder cells. In some embodiments, the PBMC are irradiated with gamma rays in the range of 3000 to 3600 rads to prevent cell division. In some embodiments, the PBMC are irradiated with gamma rays of 3000, 3100, 3200, 3300, 3400, 3500 or 3600 rads or any value of rads between any two endpoints of any of the listed values to prevent cell division. The order of addition of the T cells and feeder cells to the culture media can be reversed if desired. The culture can typically be incubated under conditions of temperature and the like that are suitable for the growth of T lymphocytes. For the growth of human T lymphocytes, for example, the temperature will generally be at least 25° C., preferably at least 30° C., more preferably 37° C. In some embodiments, the temperature for the growth of human T lymphocytes is 22, 24, 26, 28, 30, 32, 34, 36, 37° C., or any other temperature between any two endpoints of any of the listed values.

After isolation of T lymphocytes both cytotoxic and helper T lymphocytes can be sorted into naïve, memory, and effector T cell subpopulations either before or after expansion.

CD8+ cells can be obtained by using standard methods. In some embodiments, CD8+ cells are further sorted into naïve, central memory, and effector memory cells by identifying cell surface antigens that are associated with each of those types of CD8+ cells. In some embodiments, memory T cells are present in both CD62L+ and CD62L− subsets of CD8+ peripheral blood lymphocytes. PBMC are sorted into CD62L-CD8+ and CD62L+CD8+ fractions after staining with anti-CD8 and anti-CD62L antibodies. In some embodiments, the expression of phenotypic markers of central memory T_{CM} include CD45RO, CD62L, CCR7, CD28, CD3, and/or CD127 and are negative or low for granzyme B. In some embodiments, central memory T cells are CD45R0+, CD62L+, and/or CD8+ T cells. In some embodiments, effector T_{E} are negative for CD62L, CCR7, CD28, and/or CD127, and positive for granzyme B and/or perforin. In some embodiments, naïve CD8+T lymphocytes are characterized by the expression of phenotypic markers of naïve T cells comprising CD62L, CCR7, CD28, CD3, CD127, and/or CD45RA.

CD4+T helper cells are sorted into naïve, central memory, and effector cells by identifying cell populations that have cell surface antigens. CD4+ lymphocytes can be obtained by standard methods. In some embodiments, naïve CD4+T lymphocytes are CD45R0-, CD45RA+, CD62L+, and/or CD4+ T cells. In some embodiments, central memory CD4+ cells are CD62L+ and/or CD45RO+. In some embodiments, effector CD4+ cells are CD62L− and/or CD45RO−.

Whether a cell, such as a mammalian cell, or cell population, such as a population of mammalian cells, is selected for expansion depends upon whether the cell or population of cells has undergone two distinct genetic modification events. If a cell, such as a mammalian cell, or a population of cells, such as a population of mammalian cells, has undergone one or fewer genetic modification events, then the addition of a ligand will result in no dimerization. However, if the cell, such as a mammalian cell, or the population of cells, such as a population of mammalian cells, has undergone two genetic modification events, then the addition of the ligand will result in dimerization of the CISC component, and subsequent signaling cascade. Thus, a cell, such as a mammalian cell, or a population of cells, such as a population of mammalian cells, may be selected based on its response to contact with the ligand. In some embodiments, the ligand may be added in an amount of 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 20, 25, 30, 35, 40, 45, 50, 55, 60, 65, 70, 75, 80, 85, 90, 95, or 100 nM or a concentration within a range defined by any two of the aforementioned values.

In some embodiments, a cell, such as a mammalian cell, or a population of cells, such as a population of mammalian cells, may be positive for the dimeric CISC based on the expression of a marker as a result of a signaling pathway. Thus, a cell population positive for the dimeric CISC may be determined by flow cytometry using staining with a specific antibody for the surface marker and an isotype matched control antibody.

In some embodiments, the genetically modified cells comprising the protein sequences of any one of the embodiments described herein or the expression vector of any one of the embodiments described herein comprises a phenotype similar to a naturally occurring thymic T_{reg} (tT_{reg}). Such a genetically modified cell is also referred to herein as an “edT_{reg}.” In some embodiments, the edT_{regs} are characterized by i) high levels of one or more (such as any of 2, 3, 4, or 5) of FOXP3, CD25, CTLA4, ICOS, and LAG3, and/or ii) low levels of CD127. In some embodiments, the edT_{regs} are characterized by high levels of FOXP3, CD25, CTLA4, ICOS, and LAG3, and low levels of CD127. In some embodiments, the edT_{regs} have a memory phenotype. In some embodiments, the edT_{regs} are characterized by high levels of CD45RO. In some embodiments, the edT_{regs} are characterized by low levels of Helios. In some embodiments, the edT_{regs} are characterized in that they have a reduced inflammatory cytokine response to stimulation as compared to corresponding cells that have not been genetically modified. In some embodiments, the edT_{regs} are characterized in that they have a reduced IL-2, IFNγ, and/or TNFα response to stimulation as compared to corresponding cells that have not been genetically modified. In some embodiments, the edT_{regs} are characterized in that they have a reduced IL-2, IFNγ, and TNFα response to stimulation as compared to corresponding cells that have not been genetically modified.

In some embodiments, the genetically modified cells comprising the protein sequences of any one of the embodiments described herein or the expression vector of any one of the embodiments described herein can be enriched by known techniques, such as affinity binding. For example, genetically modified cells expressing LNGFR can be enriched by affinity binding to an LNGFR-selective material, such as beads conjugated with an anti-LNGFR antibody or a binding fragment thereof.

In some embodiments, the genetically modified cells are edT_{regs}, and are characterized in that administration of the edT_{regs} to a mouse model of graft vs. host disease (GVHD) results in delay of onset of GVHD in the mouse model and/or increased survival of the mouse model as compared to a corresponding mouse model administered corresponding cells that were not genetically modified. In some embodiments, the edT_{regs} are administered to the mouse model by intraperitoneal route or intravenous route. In some embodiments, the mouse model is administered a cell composition comprising at least at or about 60% (such as at least at or about any of 65%, 70%, 75%, 80%, 85%, 90%, 95%, 96%, 97%, 98%, 99%, or greater) edT_{regs}. In some embodiments, the mouse model is administered a cell composition comprising at at or about 70% edT_{regs}. In some embodiments, the mouse model is administered a cell composition comprising at at or about 90% edTregs.

In some embodiments, the cell is not a germ cell.

### Methods of Making

A method of making a genetically engineered cell is provided. The method comprises the steps: providing a cell, wherein the cell comprises a first nucleic acid comprising at least one targeted locus, providing a CAS9 protein or a second nucleic acid encoding a CAS9 protein, introducing the CAS9 protein or the second nucleic acid into the cell, introducing a third nucleic acid encoding at least one CRISPR guide sequence or a set of nucleic acids encoding at least one CRISPR guide sequence, wherein the at least one CRISPR guide sequence is configured to hybridize to the at least one targeted locus and introducing a fourth nucleic acid into the cell, wherein the fourth nucleic acid comprises a gene delivery cassette.

In some embodiments, the method further comprises activating the cell, wherein the activating is performed before the introducing of the second nucleic acid into the cell. Activating may performed by contacting the cell with CD3 and/or CD28. The CD3 and/or CD28 may be comprised on a solid support such as a bed.

In some embodiments, the at least one targeted locus is a FOXP3 locus, AAVS1 locus or a TCRa (TRAC) locus. In some embodiments, the second nucleic acid, third nucleic acid, the set of nucleic acids and/or the fourth nucleic acid is provided in one or more vectors.

In some embodiments, the one or more vectors is a viral vector. In some embodiments, the viral vector is an Adeno-associated virus (AAV) vector. In some embodiments, the AAV vector is a self-complementary vector. In some embodiments, the AAV vector is a single stranded vector. In some embodiments, the AAV vector is a combination of a self-complementary vector and a single stranded vector.

In some embodiments, the second nucleic acid encoding the CAS9 protein is an mRNA. In some embodiments, the at least one guide sequence comprises a sequence set forth in any one of SEQ ID NOs: 1-7, 15-20, 27-29, 33 and/or 34. In some embodiments, the second nucleic acid, the third nucleic acid, the set of nucleic acids and/or the fourth nucleic acid are codon optimized for expression in a eukaryotic cell, such as a human cell. Codon optimization, is understood by those skilled in the art, and nucleic acids may be optimized by computational methods.

In some embodiments, the fourth nucleic acid comprises a sequence encoding a human codon optimized FOXP3 cDNA sequence.

In some embodiments, the fourth nucleic acid sequence comprises a sequence set forth in SEQ ID NO: 68 or 69. In some embodiments, the fourth nucleic acid further comprises a promoter. In some embodiments, the promoter is a MND promoter, PGK promoter or an E2F promoter.

In some embodiments, the fourth nucleic acid further comprises a sequence encoding a low affinity nerve growth factor receptor coding sequence (LNGFR), μCISC, CISCγ, FRB and/or LNGFRe (LNGFR epitope coding sequence). The LNGFR may be used as a marker for enrichment of cells.

The cells having μCISC, CISCγ, FRB may be used in compositions and methods, which would allow the use of rapamycin-mediated CISC intracellular signaling but which remediates the negative effects that rapamycin or rapamycin-related compounds have on the growth and viability of host cells carrying the FOXP3 gene.

In some embodiments, the method further comprises introducing a fifth nucleic into the cell, wherein the fifth nucleic acid comprises a second gene delivery cassette. In some embodiments, the fifth nucleic acid is provided in a vector. In some embodiments, the vector is an AAV vector. In some embodiments, the fifth nucleic acid comprises a sequence encoding CISC, FRB, a marker protein, μCISC, and/or βCISC.

In some embodiments, the fourth and or fifth sequence further comprises a sequence encoding a P2A self-cleaving peptide. In some embodiments, the fourth and or fifth sequence further comprises a sequence encoding a polyA sequence. In some embodiments, the polyA sequence comprises a SV40polyA or 3′UTR of FOXP3. In some embodiments, the fourth sequence comprises a sequence as set forth in any one of SEQ ID NO: 37-42. In some embodiments, a fourth sequence and a fifth sequence are introduced into the cell, wherein the fourth and fifth sequence comprise a sequence as set forth in SEQ ID NO: 37 and 43, SEQ ID NO: 37 and 44, SEQ ID NO: 38 and 43, SEQ ID NO: 38 and 44, SEQ ID NO: 45 and 46, or SEQ ID NO: 45 and 47, respectively.

In some embodiments, the cell is a primary human lymphocyte.

In some embodiments, the fourth nucleic acid comprises at least one homology arm with a locus specific sequence and, wherein the homology arm length is configured for efficient packaging into an AAV vector. The homology arm may be configured to add additional genes to the construct.

In some embodiments, the at least one homology arm comprises a length of 0.25, 0.3, 0.45, 0.6 or 0.8 kb or any length in between a range defined by any two aforementioned values. In some embodiments, the marker is LNGF, RQR8 or EGFRt.

In some embodiments, the method further comprises introducing into the cell a sixth nucleic acid encoding a protein or cytokine for co-expression with FOXP3. In some embodiments, the method further comprises selecting the cells by enrichment of the marker.

In some embodiments, the method is carried out on an input population of cells to generate an output population of cells, wherein one or more cells in the output cell population are modified. In some embodiments, the modified cells in the output cell population express a surface marker (e.g., LNGFR) that is not expressed in the unmodified cells in the output cell population. In some embodiments, the method further comprises enriching the output cell population for the modified cells. The modified cells can be enriched by known techniques, such as affinity binding. For example, modified cells expressing LNGFR can be enriched by affinity binding to an LNGFR-selective material, such as beads conjugated with an anti-LNGFR antibody. Enriching for the modified cells allows for obtaining a higher yield and purity of the modified cells following subsequent expansion. In some embodiments, enriching the output cell population for the modified cells results in an enriched population of cells comprising at least at or about 90% (such as at least at or about any of 91%, 92%, 93%, 94%, 95%, 96%, 97%, 98%, 99%, or greater) modified cells (e.g., LNGFR+modified cells).

A cell for expression of FOXP3 is also provided, wherein the cell is manufactured by the method of any one of the embodiments described herein. In some embodiments, the cell is a primary human lymphocyte. In some embodiments, FOXP3 is expressed constitutively or the expression is regulated.

In some embodiments, a cell for expression of FOXP3 is provided, the cell comprising: a nucleic acid encoding a gene encoding a FOXP3. In some embodiments, the gene encoding a FOXP3 is integrated at a FOXP3 or a non-FOXP3 locus. In some embodiments, the non-FOXP3 locus is an AAVS1 locus or a TCRa (TRAC) locus. In some embodiments, the cell is a primary human lymphocyte. In some embodiments, the cell expresses CISCβ: FRB-IL2Rβ, DISC, CISC-FRB, μDISC, μCISC-FRB, FRB, LNGFR and/or LNGFRe. In some embodiments, the cell comprises a T_{reg} phenotype.

In some embodiments, a composition comprising the cell of any one of the embodiments herein is provided. In some embodiments, the composition comprises a pharmaceutical excipient.

In some embodiments, a method for treating, ameliorating, and/or inhibiting a disease and/or a condition in a subject is provided, the method comprises providing to a subject having a disease and/or a condition the cells or the composition of any one of embodiments herein. In some embodiments, providing the cells to the subject suppresses or inhibits an immune response in the subject. In some embodiments, the immune response that is suppressed or inhibited is a T cell-mediated inflammatory response.

In some embodiments, the disease is an autoimmune disease. In some embodiments, the disease is X-linked (IPEX) syndrome. In some embodiments, the condition is Graft-versus Host Disease (GVHD). In some embodiments, the condition is one associated with a solid organ transplant.

In some embodiments, a method of making a genetically engineered cell is provided, the method comprising: providing a cell, wherein the cell comprises a first nucleic acid comprising at least one targeted locus; providing a CAS9 protein or a second nucleic acid encoding a CAS9 protein; introducing the CAS9 protein or the second nucleic acid into the cell; introducing a third nucleic acid encoding at least one CRISPR guide sequence or a set of nucleic acids encoding at least one CRISPR guide sequence, wherein the at least one CRISPR guide sequence is configured to hybridize to the at least one targeted locus; and introducing a fourth nucleic acid into the cell, wherein the fourth nucleic acid comprises a gene delivery cassette. In some embodiments, the method further comprises activating the cell, wherein the activating is performed before the introducing of the second nucleic acid into the cell. In some embodiments, the activating is performed by contacting the cell with CD3 and/or CD28. In some embodiments, the at least one targeted locus is a FOXP3 locus, AAVS1 locus or a TCRa (TRAC) locus. In some embodiments, the second nucleic acid, third nucleic acid, the set of nucleic acids and/or the fourth nucleic acid is provided in one or more vectors. In some embodiments, the one or more vectors is a viral vector. In some embodiments, the viral vector is an Adeno-associated virus (AAV) vector. In some embodiments, the AAV vector is a self-complementary vector. In some embodiments, the AAV vector is a single stranded vector. In some embodiments, the AAV vector is a combination of a self-complementary vector and a single stranded vector. In some embodiments, the second nucleic acid encoding the CAS9 protein is an mRNA. In some embodiments, the at least one guide sequence comprises a sequence set forth in any one of SEQ ID NOs: 1-7, 15-20, 27-29, 33 and/or 34. In some embodiments, the second nucleic acid, the third nucleic acid, the set of nucleic acids and/or the fourth nucleic acid are codon optimized for expression in a eukaryotic cell, such as a human cell. In some embodiments, the fourth nucleic acid comprises a sequence encoding a human codon optimized FOXP3 cDNA sequence. In some embodiments, the fourth nucleic acid sequence comprises a sequence set forth in SEQ ID NO: 68 or 69. In some embodiments, the fourth nucleic acid further comprises a promoter. In some embodiments, the promoter is a MND promoter, PGK promoter or an E2F promoter. In some embodiments, the fourth nucleic acid further comprises a sequence encoding a low affinity nerve growth factor receptor coding sequence (LNGFR), μCISC, CISCγ, FRB and/or LNGFRe (LNGFR epitope coding sequence). In some embodiments, the method further comprises introducing a fifth nucleic into the cell, wherein the fifth nucleic acid comprises a second gene delivery cassette. In some embodiments, the fifth nucleic acid is provided in a vector. In some embodiments, the vector is an AAV vector. In some embodiments, the fifth nucleic acid comprises a sequence encoding CISC, FRB, a marker protein, μCISC, and/or μCISC. In some embodiments, the fourth and or fifth nucleic acid further comprises a sequence encoding a P2A self cleaving peptide. In some embodiments, the fourth and or fifth sequence further comprises a sequence encoding a polyA sequence. In some embodiments, the polyA sequence comprises a SV40polyA or 3′UTR of FOXP3. In some embodiments, the fourth sequence comprises a sequence as set forth in any one of SEQ ID NO: 37-42. In some embodiments, a fourth a fifth nucleic acid are introduced into the cell, wherein the fourth and fifth nucleic acid comprises a sequence as set forth in SEQ ID NO: 37 and 43, SEQ ID NO: 37 and 44, SEQ ID NO: 38 and 43, SEQ ID NO: 38 and 44, SEQ ID NO: 45 and 46, or SEQ ID NO: 45 and 47, respectively. In some embodiments, the cell is a primary human lymphocyte. In some embodiments, the fourth nucleic acid comprises at least one homology arm with a locus specific sequence and, wherein the homology arm length is configured for efficient packaging into an AAV vector. In some embodiments, the at least one homology arm comprises a length of 0.25, 0.3, 0.45, 0.6 or 0.8 kb or any length in between a range defined by any two aforementioned values. In some embodiments, the marker is LNGF, RQR8 or EGFRt. In some embodiments, the method further comprises introducing into the cell a sixth nucleic acid encoding a protein or cytokine for co-expression with FOXP3. In some embodiments, the protein of cytokine is a T cell receptor, a chimeric antigen receptor or IL-10. In some embodiments, the method further comprises selecting the cells by enrichment of the marker.

In some embodiments, a cell for expression of FOXP3 is provided, manufactured by the method of any one of the embodiments herein. In some embodiments, the method comprises providing a cell, wherein the cell comprises a first nucleic acid comprising at least one targeted locus; providing a CAS9 protein or a second nucleic acid encoding a CAS9 protein; introducing the CAS9 protein or the second nucleic acid into the cell; introducing a third nucleic acid encoding at least one CRISPR guide sequence or a set of nucleic acids encoding at least one CRISPR guide sequence, wherein the at least one CRISPR guide sequence is configured to hybridize to the at least one targeted locus; and introducing a fourth nucleic acid into the cell, wherein the fourth nucleic acid comprises a gene delivery cassette. In some embodiments, the method further comprises activating the cell, wherein the activating is performed before the introducing of the second nucleic acid into the cell. In some embodiments, the activating is performed by contacting the cell with CD3 and/or CD28. In some embodiments, the at least one targeted locus is a FOXP3 locus, AAVS1 locus or a TCRa (TRAC) locus. In some embodiments, the second nucleic acid, third nucleic acid, the set of nucleic acids and/or the fourth nucleic acid is provided in one or more vectors. In some embodiments, the one or more vectors is a viral vector. In some embodiments, the viral vector is an Adeno-associated virus (AAV) vector. In some embodiments, the AAV vector is a self-complementary vector. In some embodiments, the AAV vector is a single stranded vector. In some embodiments, the AAV vector is a combination of a self-complementary vector and a single stranded vector. In some embodiments, the second nucleic acid encoding the CAS9 protein is an mRNA. In some embodiments, the at least one guide sequence comprises a sequence set forth in any one of SEQ ID NOs: 1-7, 15-20, 27-29, 33 and/or 34. In some embodiments, the second nucleic acid, the third nucleic acid, the set of nucleic acids and/or the fourth nucleic acid are codon optimized for expression in a eukaryotic cell, such as a human cell. In some embodiments, the fourth nucleic acid comprises a sequence encoding a human codon optimized FOXP3 cDNA sequence. In some embodiments, the fourth nucleic acid sequence comprises a sequence set forth in SEQ ID NO: 68 or 69. In some embodiments, the fourth nucleic acid further comprises a promoter. In some embodiments, the promoter is a MND promoter, PGK promoter or an E2F promoter. In some embodiments, the fourth nucleic acid further comprises a sequence encoding a low affinity nerve growth factor receptor coding sequence (LNGFR), μCISC, CISCγ, FRB and/or LNGFRe (LNGFR epitope coding sequence). In some embodiments, the method further comprises introducing a fifth nucleic into the cell, wherein the fifth nucleic acid comprises a second gene delivery cassette. In some embodiments, the fifth nucleic acid is provided in a vector. In some embodiments, the vector is an AAV vector. In some embodiments, the fifth nucleic acid comprises a sequence encoding CISC, FRB, a marker protein, μCISC, and/or βCISC. In some embodiments, the fourth and or fifth nucleic acid further comprises a sequence encoding a P2A self cleaving peptide. In some embodiments, the fourth and or fifth sequence further comprises a sequence encoding a polyA sequence. In some embodiments, the polyA sequence comprises a SV40polyA or 3′UTR of FOXP3. In some embodiments, the fourth sequence comprises a sequence as set forth in any one of SEQ ID NO: 37-42. In some embodiments, a fourth a fifth nucleic acid are introduced into the cell, wherein the fourth and fifth nucleic acid comprises a sequence as set forth in SEQ ID NO: 37 and 43, SEQ ID NO: 37 and 44, SEQ ID NO: 38 and 43, SEQ ID NO: 38 and 44, SEQ ID NO: 45 and 46, or SEQ ID NO: 45 and 47, respectively. In some embodiments, the cell is a primary human lymphocyte. In some embodiments, the fourth nucleic acid comprises at least one homology arm with a locus specific sequence and, wherein the homology arm length is configured for efficient packaging into an AAV vector. In some embodiments, the at least one homology arm comprises a length of 0.25, 0.3, 0.45, 0.6 or 0.8 kb or any length in between a range defined by any two aforementioned values. In some embodiments, the marker is LNGF, RQR8 or EGFRt. In some embodiments, the method further comprises introducing into the cell a sixth nucleic acid encoding a protein or cytokine for co-expression with FOXP3. In some embodiments, the protein of cytokine is a T cell receptor, a chimeric antigen receptor or IL-10. In some embodiments, the method further comprises selecting the cells by enrichment of the marker. In some embodiments, the cell is a primary human lymphocyte. In some embodiments, FOXP3 is expressed constitutively or the expression is regulated.

In some embodiments, a cell for expression of FOXP3 is provided, the cell comprising: a nucleic acid encoding a gene encoding a FOXP3. In some embodiments, the gene encoding a FOXP3 is integrated at a FOXP3 or a non-FOXP3 locus. In some embodiments, the non-FOXP3 locus is an AAVS1 locus or a TCRa (TRAC) locus. In some embodiments, the cell is a primary human lymphocyte. In some embodiments, the cell expresses CISCβ: FRB-IL2Rβ, DISC, CISC-FRB, μDISC, μCISC-FRB, FRB, LNGFR and/or LNGFRe. In some embodiments, the cell comprises a T_{reg} phenotype.

In some embodiments, a composition comprising the cell of any one of the embodiments herein is provided. In some embodiments, the cell is manufactured by the method of any one of the embodiments herein. In some embodiments, the method comprises providing a cell, wherein the cell comprises a first nucleic acid comprising at least one targeted locus; providing a CAS9 protein or a second nucleic acid encoding a CAS9 protein; introducing the CAS9 protein or the second nucleic acid into the cell; introducing a third nucleic acid encoding at least one CRISPR guide sequence or a set of nucleic acids encoding at least one CRISPR guide sequence, wherein the at least one CRISPR guide sequence is configured to hybridize to the at least one targeted locus; and introducing a fourth nucleic acid into the cell, wherein the fourth nucleic acid comprises a gene delivery cassette. In some embodiments, the method further comprises activating the cell, wherein the activating is performed before the introducing of the second nucleic acid into the cell. In some embodiments, the activating is performed by contacting the cell with CD3 and/or CD28. In some embodiments, the at least one targeted locus is a FOXP3 locus, AAVS1 locus or a TCRa (TRAC) locus. In some embodiments, the second nucleic acid, third nucleic acid, the set of nucleic acids and/or the fourth nucleic acid is provided in one or more vectors. In some embodiments, the one or more vectors is a viral vector. In some embodiments, the viral vector is an Adeno-associated virus (AAV) vector. In some embodiments, the AAV vector is a self-complementary vector. In some embodiments, the AAV vector is a single stranded vector. In some embodiments, the AAV vector is a combination of a self-complementary vector and a single stranded vector. In some embodiments, the second nucleic acid encoding the CAS9 protein is an mRNA. In some embodiments, the at least one guide sequence comprises a sequence set forth in any one of SEQ ID NOs: 1-7, 15-20, 27-29, 33 and/or 34. In some embodiments, the second nucleic acid, the third nucleic acid, the set of nucleic acids and/or the fourth nucleic acid are codon optimized for expression in a eukaryotic cell, such as a human cell. In some embodiments, the fourth nucleic acid comprises a sequence encoding a human codon optimized FOXP3 cDNA sequence. In some embodiments, the fourth nucleic acid sequence comprises a sequence set forth in SEQ ID NO: 68 or 69. In some embodiments, the fourth nucleic acid further comprises a promoter. In some embodiments, the promoter is a MND promoter, PGK promoter or an E2F promoter. In some embodiments, the fourth nucleic acid further comprises a sequence encoding a low affinity nerve growth factor receptor coding sequence (LNGFR), μCISC, CISCγ, FRB and/or LNGFRe (LNGFR epitope coding sequence). In some embodiments, the method further comprises introducing a fifth nucleic into the cell, wherein the fifth nucleic acid comprises a second gene delivery cassette. In some embodiments, the fifth nucleic acid is provided in a vector. In some embodiments, the vector is an AAV vector. In some embodiments, the fifth nucleic acid comprises a sequence encoding CISC, FRB, a marker protein, μCISC, and/or βCISC. In some embodiments, the fourth and or fifth nucleic acid further comprises a sequence encoding a P2A self cleaving peptide. In some embodiments, the fourth and or fifth sequence further comprises a sequence encoding a polyA sequence. In some embodiments, the polyA sequence comprises a SV40polyA or 3′UTR of FOXP3. In some embodiments, the fourth sequence comprises a sequence as set forth in any one of SEQ ID NO: 37-42. In some embodiments, a fourth a fifth nucleic acid are introduced into the cell, wherein the fourth and fifth nucleic acid comprises a sequence as set forth in SEQ ID NO: 37 and 43, SEQ ID NO: 37 and 44, SEQ ID NO: 38 and 43, SEQ ID NO: 38 and 44, SEQ ID NO: 45 and 46, or SEQ ID NO: 45 and 47, respectively. In some embodiments, the cell is a primary human lymphocyte. In some embodiments, the fourth nucleic acid comprises at least one homology arm with a locus specific sequence and, wherein the homology arm length is configured for efficient packaging into an AAV vector. In some embodiments, the at least one homology arm comprises a length of 0.25, 0.3, 0.45, 0.6 or 0.8 kb or any length in between a range defined by any two aforementioned values. In some embodiments, the marker is LNGF, RQR8 or EGFRt. In some embodiments, the method further comprises introducing into the cell a sixth nucleic acid encoding a protein or cytokine for co-expression with FOXP3. In some embodiments, the protein of cytokine is a T cell receptor, a chimeric antigen receptor or IL-10. In some embodiments, the method further comprises selecting the cells by enrichment of the marker. In some embodiments, the cell is a primary human lymphocyte. In some embodiments, FOXP3 is expressed constitutively or the expression is regulated.

In some embodiments, a method for treating, ameliorating, and/or inhibiting a disease and/or a condition in a subject is provided, the method comprising: providing to a subject having a disease and/or a condition the cell or the composition of any of the embodiments herein. In some embodiments, the cell is manufactured by the method of any one of the embodiments herein. In some embodiments, the method comprises providing a cell, wherein the cell comprises a first nucleic acid comprising at least one targeted locus; providing a CAS9 protein or a second nucleic acid encoding a CAS9 protein; introducing the CAS9 protein or the second nucleic acid into the cell; introducing a third nucleic acid encoding at least one CRISPR guide sequence or a set of nucleic acids encoding at least one CRISPR guide sequence, wherein the at least one CRISPR guide sequence is configured to hybridize to the at least one targeted locus; and introducing a fourth nucleic acid into the cell, wherein the fourth nucleic acid comprises a gene delivery cassette. In some embodiments, the method further comprises activating the cell, wherein the activating is performed before the introducing of the second nucleic acid into the cell. In some embodiments, the activating is performed by contacting the cell with CD3 and/or CD28. In some embodiments, the at least one targeted locus is a FOXP3 locus, AAVS1 locus or a TCRa (TRAC) locus. In some embodiments, the second nucleic acid, third nucleic acid, the set of nucleic acids and/or the fourth nucleic acid is provided in one or more vectors. In some embodiments, the one or more vectors is a viral vector. In some embodiments, the viral vector is an Adeno-associated virus (AAV) vector. In some embodiments, the AAV vector is a self-complementary vector. In some embodiments, the AAV vector is a single stranded vector. In some embodiments, the AAV vector is a combination of a self-complementary vector and a single stranded vector. In some embodiments, the second nucleic acid encoding the CAS9 protein is an mRNA. In some embodiments, the at least one guide sequence comprises a sequence set forth in any one of SEQ ID NOs: 1-7, 15-20, 27-29, 33 and/or 34. In some embodiments, the second nucleic acid, the third nucleic acid, the set of nucleic acids and/or the fourth nucleic acid are codon optimized for expression in a eukaryotic cell, such as a human cell. In some embodiments, the fourth nucleic acid comprises a sequence encoding a human codon optimized FOXP3 cDNA sequence. In some embodiments, the fourth nucleic acid sequence comprises a sequence set forth in SEQ ID NO: 68 or 69. In some embodiments, the fourth nucleic acid further comprises a promoter. In some embodiments, the promoter is a MND promoter, PGK promoter or an E2F promoter. In some embodiments, the fourth nucleic acid further comprises a sequence encoding a low affinity nerve growth factor receptor coding sequence (LNGFR), μCISC, CISCγ, FRB and/or LNGFRe (LNGFR epitope coding sequence). In some embodiments, the method further comprises introducing a fifth nucleic into the cell, wherein the fifth nucleic acid comprises a second gene delivery cassette. In some embodiments, the fifth nucleic acid is provided in a vector. In some embodiments, the vector is an AAV vector. In some embodiments, the fifth nucleic acid comprises a sequence encoding CISC, FRB, a marker protein, μCISC, and/or βCISC. In some embodiments, the fourth and or fifth nucleic acid further comprises a sequence encoding a P2A self cleaving peptide. In some embodiments, the fourth and or fifth sequence further comprises a sequence encoding a polyA sequence. In some embodiments, the polyA sequence comprises a SV40polyA or 3′UTR of FOXP3. In some embodiments, the fourth sequence comprises a sequence as set forth in any one of SEQ ID NO: 37-42. In some embodiments, a fourth a fifth nucleic acid are introduced into the cell, wherein the fourth and fifth nucleic acid comprises a sequence as set forth in SEQ ID NO: 37 and 43, SEQ ID NO: 37 and 44, SEQ ID NO: 38 and 43, SEQ ID NO: 38 and 44, SEQ ID NO: 45 and 46, or SEQ ID NO: 45 and 47, respectively. In some embodiments, the cell is a primary human lymphocyte. In some embodiments, the fourth nucleic acid comprises at least one homology arm with a locus specific sequence and, wherein the homology arm length is configured for efficient packaging into an AAV vector. In some embodiments, the at least one homology arm comprises a length of 0.25, 0.3, 0.45, 0.6 or 0.8 kb or any length in between a range defined by any two aforementioned values. In some embodiments, the marker is LNGF, RQR8 or EGFRt. In some embodiments, the method further comprises introducing into the cell a sixth nucleic acid encoding a protein or cytokine for co-expression with FOXP3. In some embodiments, the protein of cytokine is a T cell receptor, a chimeric antigen receptor or IL-10. In some embodiments, the method further comprises selecting the cells by enrichment of the marker. In some embodiments, the cell is a primary human lymphocyte. In some embodiments, FOXP3 is expressed constitutively or the expression is regulated. In some embodiments, providing the cells to the subject suppresses or inhibits an immune response in the subject. In some embodiments, the immune response that is suppressed or inhibited is a T cell-mediated inflammatory response. In some embodiments, the disease is an autoimmune disease. In some embodiments, the disease is X-linked (IPEX) syndrome. In some embodiments, the condition is Graft-versus Host Disease (GVHD). In some embodiments, the subject has a solid organ transplant.

Method of Making a Cell that Expresses a Dimeric CISC Component

In some embodiments described herein, it may be desired to introduce a protein sequence or an expression vector into a host cell, such as a mammalian cell, e.g., a lymphocyte, to be used for drug regulated cytokine signaling and/or for the selective expansion of cells that express the dimeric CISC components. For example, the dimeric CISC can allow for cytokine signaling in cells that have the introduced CISC components for transmitting signals to the interior of a cell, such as a mammalian cell, upon contact with a ligand. In addition, the selective expansion of cells, such as mammalian cells, can be controlled to select for only those cells that have undergone two specific genetic modification events, as described herein. Preparation of these cells can be carried out in accordance with known techniques that will be apparent to those skilled in the art based upon the present disclosure.

In some embodiments, a method of making a CISC-bearing cell, such as a mammalian cell, is provided, wherein the cell expresses a dimeric CISC. The method can include delivering to a cell, such as a mammalian cell, the protein sequence of any one of the embodiments or the expression vector of the embodiments described herein and delivering to the cell, such as a mammalian cell. In some embodiments, the protein sequence comprises a first and a second sequence. In some embodiments, the first sequence encodes for a first CISC component comprising a first extracellular binding domain, a hinge domain, a linker of a specified length, wherein the length is preferably optimized, a transmembrane domain, and a signaling domain. In some embodiments, the second sequence encodes for a second CISC component comprising a second extracellular binding domain, a hinge domain, a linker of a specified length, wherein the length is preferably optimized, a transmembrane domain, and a signaling domain. In some embodiments, the spacer is 1, 2, 3, 4, 5, 6, 7, 8, 9, 10, 11, 12, 13, 14 or 15 amino acids in length or a length within a range defined by any two of the aforementioned lengths. In some embodiments, the signaling domain comprises an interleukin-2 signaling domain, such as an IL2Rb or an IL2Rg domain. In some embodiments, the extracellular binding domain is a binding domain that binds to rapamycin or a rapalog, comprising FKBP or FRB or a portion thereof. In some embodiments, the cell is a CD8+ or a CD4+ cell. In some embodiments, the cell is a CD8+T cytotoxic lymphocyte cell selected from the group consisting of naïve CD8+ T cells, central memory CD8+ T cells, effector memory CD8+ T cells and bulk CD8+ T cells. In some embodiments, the cell is a CD4+T helper lymphocyte cell that is selected from the group consisting of naïve CD4+ T cells, central memory CD4+ T cells, effector memory CD4+ T cells, and bulk CD4+ T cells. In some embodiments, the cell is a precursor T cell. In some embodiments, the cell is a stem cell. In some embodiments, the cell is a hematopoietic stem cell. In some embodiments, the cell is a B cell. In some embodiments, the cell is a neuronal stem cell. In some embodiments, the cell is an NK cell.

### Method of Activating a Signal in the Interior of a Cell

In some embodiments, a method of activating a signal in the interior of a cell, such as a mammalian cell, is provided. The method can include providing a cell, such as a mammalian cell, as described herein, wherein the cell comprises a protein sequence as set forth herein or an expression vector as set forth herein. In some embodiments, the method further comprises expressing the protein sequence encoding a dimeric CISC as described herein, or expression the vector as described herein. In some embodiments, the method comprises contacting the cell, such as a mammalian cell, with a ligand, which causes the first and second CISC components to dimerize, which transduces a signal into the interior of the cell. In some embodiments, the ligand is rapamycin or rapalog. In some embodiments, the ligand is an IMID-class drug (e.g. thalidomide, pomalidomide, or lenalidomide or related analogues). In some embodiments an effective amount of a ligand for inducing dimerization is provided an amount of 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 20, 25, 30, 35, 40, 45, 50, 55, 60, 65, 70, 75, 80, 85, 90, 95, or 100 nM or a concentration within a range defined by any two of the aforementioned values.

In some embodiments, the ligand used in these approaches is rapamycin or a rapalog, comprising, for example, everolimus, CCI-779, C20-methallylrapamycin, C16-(S)-3-methylindolerapamycin, C16-iRap, AP21967, sodium mycophenolic acid, benidipine hydrochloride, AP23573, or AP1903, or metabolites, derivatives, and/or combinations thereof. Additional useful rapalogs may include, for example, variants of rapamycin having one or more of the following modifications relative to rapamycin: demethylation, elimination or replacement of the methoxy at C7, C42 and/or C29; elimination, derivatization or replacement of the hydroxy at C13, C43 and/or C28; reduction, elimination or derivatization of the ketone at C14, C24 and/or C30; replacement of the 6-membered pipecolate ring with a 5-membered prolyl ring; and/or other substitution on the cyclohexyl ring or replacement of the cyclohexyl ring with a substituted cyclopentyl ring. Additional useful rapalogs may include novolimus, pimecrolimus, ridaforolimus, tacrolimus, temsirolimus, umirolimus, or zotarolimus, or metabolites, derivatives, and/or combinations thereof. In some embodiments, the ligand is an IMID-class drug (e.g. thalidomide, pomalidomide, lenalidomide or related analogues).

In some embodiments, detecting a signal in the interior of the cell, such as a mammalian cell, can be achieved by a method of detecting a marker that is the result of a signaling pathway. Thus, for example, a signal may be detected by determining the levels of Akt or other signaling marker in a cell, such as a mammalian cell, through a process of Western blot, flow cytometry, or other protein detection and quantification method. Markers for detection may include, for example, JAK, Akt, STAT, NF-κ, MAPK, PI3K, JNK, ERK, or Ras, or other cellular signaling markers that are indicative of a cellular signaling event.

In some embodiments, transduction of a signal affects cytokine signaling. In some embodiments, transduction of the signal affects IL2R signaling. In some embodiments, transduction of the signal affects phosphorylation of a downstream target of a cytokine receptor. In some embodiments, the method of activating a signal induces proliferation in CISC-expressing cells, such as mammalian cells, and a concomitant anti-proliferation in non-CISC expressing cells.

For cellular signaling to take place, not only must cytokine receptors dimerize or heterodimerize, but they must be in the proper configuration for a conformational change to take place (Kim, M. J. et al. (2007). NMR Structural Studies of Interactions of a Small, Nonpeptidyl Tpo Mimic with the Thrombopoietin Receptor Extracellular Juxtamembrane and Transmembrane Domains, J. Biol. Chem., 282(19):14253-14261). Thus, dimerization in conjunction with the correct conformational positioning of signaling domains are desired processes for appropriate signaling, because receptor dimerization or heterodimerization alone is insufficient to drive receptor activation. The chemical-induced signaling complexes described herein are preferably in the correct orientation for downstream signaling events to occur.

### Method of Selective Expansion of Cell Populations

In some embodiments, a method of selectively expanding a population of cells, such as mammalian cells, is provided. In some embodiments, the method comprises providing a cell, such as a mammalian cell, as described herein, wherein the cell comprises a protein sequence as set forth herein or an expression vector as set forth herein. In some embodiments, the method further comprises expressing the protein sequence encoding a dimeric CISC as described herein, or expression the vector as described herein.

In some embodiments, the method comprises contacting the cell, such as a mammalian cell, with a ligand, which causes the first and second CISC components to dimerize, which transduces a signal into the interior of the cell. In some embodiments, the ligand is rapamycin or rapalog.

In some embodiments an effective amount of a ligand provided for inducing dimerization is an amount of 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 20, 25, 30, 35, 40, 45, 50, 55, 60, 65, 70, 75, 80, 85, 90, 95, or 100 nM or a concentration within a range defined by any two of the aforementioned values.

In some embodiments, the ligand used is rapamycin or a rapalog, comprising, for example, everolimus, CCI-779, C20-methallylrapamycin, C16-(S)-3-methylindolerapamycin, C16-iRap, AP21967, sodium mycophenolic acid, benidipine hydrochloride, or AP23573, AP1903, or metabolites, derivatives, and/or combinations thereof. Additional useful rapalogs may include, for example, variants of rapamycin having one or more of the following modifications relative to rapamycin: demethylation, elimination or replacement of the methoxy at C7, C42 and/or C29; elimination, derivatization or replacement of the hydroxy at C13, C43 and/or C28; reduction, elimination or derivatization of the ketone at C14, C24 and/or C30; replacement of the 6-membered pipecolate ring with a 5-membered prolyl ring; and/or other substitution on the cyclohexyl ring or replacement of the cyclohexyl ring with a substituted cyclopentyl ring. Additional useful rapalogs may include novolimus, pimecrolimus, ridaforolimus, tacrolimus, temsirolimus, umirolimus, or zotarolimus, or metabolites, derivatives, and/or combinations thereof. In some embodiments, the ligand is an IMID-class drug (e.g. thalidomide, pomalidomide, lenalidomide or related analogues).

In some embodiments, the selective expansion of a population of cells, such as mammalian cells, takes place only when two distinct genetic modification events have taken place. One genetic modification event is one component of the dimeric chemical-induced signaling complex, and the other genetic modification event is the other component of the dimeric chemical-induced signaling complex. When both events take place within the population of cells, such as a population of mammalian cells, the chemical-induced signaling complex components dimerize in the presence of a ligand, resulting in an active chemical-induced signaling complex and generation of a signal into the interior of the cells. Other signaling markers may also be detected, but only achievement of these events in conjunction with Akt activation is able to achieve sufficient cellular expansion to allow for selective expansion of a modified cell population in which both genetic modification events have taken place in a given population of cells, such as a population of mammalian cells.

Lentiviral particles from each IL2R-CISC architecture were generated and used to transduce primary human T cells. CD4+ T cells were activated for 60 hours. The cells were then plated in a 24-well dish by plating 1 million cells per well in 1 mL medium with IL2/7/15. Lentivirus was transduced with or without beads, using 15 μL of IL2R-CISC and 3 μL of MND-GFP control with protamine sulfate at 4 μg/mL (0.5 mL medium) in a 24-well dish. The cells were then spinoculated at 800 g for 30 minutes at 33° C. followed by the addition of 1.5 mL medium after 4 hours of incubation. The transduced T cells were incubated at 37° C. for 48 hours with cytokines, including 50 ng/mL IL2, 5 ng/mL of ILS, and 5 ng/mL of IL17. The GFP signal was determined and the IL2R-CISC level of transduced T cells was determined. The transduction efficiency was from 10-30% for IL2R-CISC and at or about 80% for MND-GFP.

Following transduction, the cells were grown for 2 days in IL2, and then divided in half, with half grown in IL2 alone and half in rapamycin alone, as indicated. T cells were treated with rapamycin (1 nM) or IL2 for 2 days, and cells were plated at 1 million cells/well in a 24-well dish with 2 mL medium. The T cell viability was determined and the expression of GFP+ population and IL2R-CISC expression was determined by using anti-FRB antibody and a secondary APC antibody.

Similar methods as described herein may be performed using additional rapamycin analogues. For example, the methods described herein were performed using AP21967.

The IL2-CISC induced signaling pathways may be analyzed to determine whether the magnitude of the signaling pathway is sufficient to produce clinically relevant activity.

It is to be understood by those of skill in the art that the architectures and/or constructs described herein are not intended to be limiting. Thus, in addition to the V1, V2, and V3 constructs described herein, and other architectures and/or constructs described herein, additional architectures and/or may be used. Briefly, the method includes thawing a PBMC3 feeder cells, and CD4+ cells were isolated in the presence of anti-CD3/CD28 beads. The beads were removed, and spinoculated with one of V4, V5, V6, or V7 at 800×g in 500 μL. Following spinoculation, 1.5 mL TCM+cytokines were added. Each construct was then treated with various conditions, including: no treatment, 100 nM AP21967, 1 nM rapamycin, or 50 ng/mL IL-2. The expansion of the cells having each construct was then measured.

In addition, the targeted knock-in of MND promoter and CISC may be tested to enrich and/or expand gene targeted T cells. Briefly, PBMC feeder cells were thawed and CD4+ cells were isolated in the presence of anti-CD3/CD28 beads. The beads were removed and Cas9/gRNA ribonucleoproteins (RNPs) were added. The construct was then treated with various conditions, including: no treatment, 10 nM AP21967, 10 nM rapamycin, or 10 nM rapamycin+5 ng/mL IL-2.

### Therapeutic Approach

In one aspect, provided herein is a gene therapy approach for treating a subject having or suspected of having a disorder or health condition associated with a FOXP3 protein by editing the genome of the subject. For example, in some embodiments, the disorder or health condition is an autoimmune disease (e.g., IPEX syndrome) or a disorder that results from organ transplant (e.g., GVHD). In some embodiments, the gene therapy approach integrates a nucleic acid comprising a sequence encoding a functional FOXP3 gene into the genome of a relevant cell type in subjects and this can provide a permanent cure for the disorder or health condition. In some embodiments, a cell type subject to the gene therapy approach in which to integrate the FOXP3-encoding sequence is a lymphocytic cell, e.g., a CD4+ T cell, because these cells can efficiently adopt a T_{reg} phenotype in the subject.

In another aspect, provided herein are cellular, ex vivo and in vivo methods for using genome engineering tools to create permanent changes to a cell genome by knocking-in a coding sequence encoding a FOXP3 or a functional derivative thereof into a gene locus in the cell genome and restoring FOXP3 activity. Such methods use endonucleases, such as CRISPR-associated (CRISPR/Cas9, Cpf1, and the like) nucleases, to permanently delete, insert, edit, correct, or replace any sequences from the cell genome or insert an exogenous sequence, e.g., a FOXP3-encoding sequence, in a genomic locus in the cell. In this way, the examples set forth in the present disclosure restore the activity of FOXP3 with a single treatment (rather than requiring the delivery of alternative therapies for the lifetime of the subject).

In some embodiments, an ex vivo cell-based therapy is performed using a lymphocytic cell that is isolated from a subject, e.g., an autologous CD4+ T cell derived from cord blood. Next, the chromosomal DNA of these cells is edited using the systems, compositions, and methods described herein. Finally, the edited cells are implanted into the subject.

One advantage of an ex vivo cell therapy approach is the ability to conduct a comprehensive analysis of the therapeutic prior to administration. All nuclease-based therapeutics have some level of off-target effects. Performing gene correction ex vivo allows one to fully characterize the corrected cell population prior to implantation. Aspects of the disclosure include sequencing the entire genome of the corrected cells to ensure that the off-target cuts, if any, are in genomic locations associated with minimal risk to the subject. Furthermore, populations of specific cells, including clonal populations, can be isolated prior to implantation.

Another embodiment of such methods is an in vivo based therapy. In this method, the chromosomal DNA of the cells in the subject is corrected using the systems, compositions, and methods described herein. In some embodiments, the cells are lymphocytic cells, e.g., CD4+ cells, such as T cells.

An advantage of in vivo gene therapy is the ease of therapeutic production and administration. The same therapeutic approach and therapy can be used to treat more than one subject, for example a number of subjects who share the same or similar genotype or allele. In contrast, ex vivo cell therapy generally uses a subject's own cells, which are isolated, manipulated, and returned to the same subject.

In some embodiments, the subject who is in need of the treatment method accordance with the disclosures is a subject having symptoms of a disease or condition associated with a FOXP3. For example, in some embodiments, the subject has symptoms of an autoimmune disease (e.g., IPEX syndrome) or a disorder that results from organ transplant (e.g., GVHD). In some embodiments, the subject can be a human suspected of having the disease or condition. Alternatively, the subject can be a human diagnosed with a risk of the disease or condition. In some embodiments, the subject who is in need of the treatment can have one or more genetic defects (e.g., deletion, insertion, and/or mutation) in the endogenous FOXP3 gene or its regulatory sequences such that the activity including the expression level or functionality of the FOXP3 is substantially reduced compared to a normal, healthy subject.

In some embodiments, provided herein is a method of treating a disease or condition associated with a FOXP3 (e.g., an autoimmune disease) in a subject, the method comprising providing the following to a cell in the subject: (a) a guide RNA (gRNA) targeting the FOXP3 locus in the cell genome; (b) a DNA endonuclease or nucleic acid encoding said DNA endonuclease; and (c) a donor template comprising a nucleic acid sequence encoding a FOXP3 or a functional derivative thereof. In some embodiments, the gRNA targets a FOXP3 locus, AAVS1 locus or a TCRa (TRAC) locus. In some embodiments, the gRNA comprises a spacer sequence from any one of SEQ ID NOs: 1-7, 15-20, 27-29, 33, and 34.

In some embodiments, provided herein is a method of treating a disease or condition associated with FOXP3 (e.g., an autoimmune disease such as IPEX syndrome) in a subject, the method comprising providing the following to a cell in the subject: (a) a gRNA comprising a spacer sequence that is complementary to a genomic sequence within or near an endogenous FOXP3 locus in the cell; (b) a DNA endonuclease or nucleic acid encoding said DNA endonuclease; and (c) a donor template comprising a nucleic acid sequence encoding the FOXP3 or a functional derivative thereof. In some embodiments, the gRNA comprises a spacer sequence from any one of SEQ ID NOs: 1-7 and 27-29 or a variant thereof having no more than 3 mismatches compared to any one of SEQ ID NOs: 1-7 and 27-29. In some embodiments, the gRNA comprises a spacer sequence from any one of SEQ ID NOs: 1-7 or a variant thereof having no more than 3 mismatches compared to any one of SEQ ID NOs: 1-7. In some embodiments, the gRNA comprises a spacer sequence from any one of SEQ ID NOs: 2, 3, and 5 or a variant thereof having no more than 3 mismatches compared to any one of SEQ ID NOs: 2, 3, and 5. In some embodiments, the gRNA comprises a spacer sequence from SEQ ID NO: 2 or a variant thereof having no more than 3 mismatches compared to SEQ ID NO: 2. In some embodiments, the gRNA comprises a spacer sequence from SEQ ID NO: 5 or a variant thereof having no more than 3 mismatches compared to SEQ ID NO: 5. In some embodiments, the cell is a human cell, e.g., a human lymphocytic cell, for example a human CD4+ T cell. In some embodiments, the subject is a patient having or suspected of having an autoimmune disease, e.g., IPEX syndrome or Graft-versus-Host disease. In some embodiments, the subject is diagnosed with a risk of an autoimmune disease, e.g., IPEX syndrome or Graft-versus-Host disease.

In some embodiments, provided herein is a method of treating a disease or condition associated with FOXP3 (e.g., an autoimmune disease) in a subject, the method comprising providing to the subject a genetically modified cell prepared by any of the methods of editing a genome in a cell described herein. In some embodiments, the nucleic acid sequence encoding a FOXP3 or a functional derivative thereof is expressed under the control of the endogenous FOXP3 promoter. In some embodiments, the nucleic acid sequence encoding a FOXP3 or a functional derivative thereof is codon-optimized for expression in the cell. In some embodiments, the nucleic acid sequence encoding a FOXP3 or a functional derivative thereof has at least at or about 70% sequence identity, e.g., at least at or about 75%, 80%, 85%, 90%, 92%, 93%, 94%, 95%, 96%, 97%, 98%, 99% or greater sequence identity, to a sequence according to SEQ ID NO: 68. In some embodiments, the cell is a lymphocytic cell. In some embodiments, the genetically modified cell is autologous to the subject. In some embodiments, the method further comprises obtaining a biological sample from the subject, wherein the biological sample comprises an input cell, and wherein the genetically modified cell is prepared from the input cell. In some embodiments, the input cell is a lymphocytic cell.

Implanting Cells into a Subject

In some embodiments, the ex vivo methods of the disclosure involve implanting the genome-edited cells into a subject who is in need of such method. This implanting step can be accomplished using any method of implantation known in the art. For example, the genetically modified cells can be injected directly in the subject's blood or otherwise administered to the subject.

In some embodiments, the methods disclosed herein include administering, which can be interchangeably used with “introducing” and “transplanting,” genetically modified, therapeutic cells into a subject, by a method or route that results in at least partial localization of the introduced cells at a desired site such that a desired effect(s) is produced. The therapeutic cells or their differentiated progeny can be administered by any appropriate route that results in delivery to a desired location in the subject where at least a portion of the implanted cells or components of the cells remain viable. The period of viability of the cells after administration to a subject can be as short as a few hours, e.g., twenty-four hours, to a few days, to as long as several years, or even the life time of the subject, such as long-term engraftment.

When provided prophylactically, the therapeutic cells described herein can be administered to a subject in advance of any symptom of a disease or condition associated with a FOXP3 (e.g., an autoimmune disease, such as IPEX syndrome). Accordingly, in some embodiments the prophylactic administration of a genetically modified stem cell population serves to prevent the occurrence of symptoms of the disease or condition.

When provided therapeutically in some embodiments, genetically modified stem cells are provided at (or after) the onset of a symptom or indication of a disease or condition associated with a FOXP3 (e.g., an autoimmune disease, such as IPEX syndrome), e.g., upon the onset of disease or condition.

For use in the various embodiments described herein, an effective amount of therapeutic cells, e.g., genome-edited stem cells, can be at least 10² cells, at least 5×10² cells, at least 10³ cells, at least 5×10³ cells, at least 10⁴ cells, at least 5×10⁴ cells, at least 10⁵ cells, at least 2×10⁵ cells, at least 3×10⁵ cells, at least 4×10⁵ cells, at least 5×10⁵ cells, at least 6×10⁵ cells, at least 7×10⁵ cells, at least 8×10⁵ cells, at least 9×10⁵ cells, at least 1×10⁶ cells, at least 2×10⁶ cells, at least 3×10⁶ cells, at least 4×10⁶ cells, at least 5×10⁶ cells, at least 6×10⁶ cells, at least 7×10⁶ cells, at least 8×10⁶ cells, at least 9×10⁶ cells, or multiples thereof. The therapeutic cells can be derived from one or more donors or can be obtained from an autologous source. In some embodiments described herein, the therapeutic cells are expanded in culture prior to administration to a subject in need thereof.

In some embodiments, modest and incremental increases in the levels of functional FOXP3 expressed in cells of subjects having a disease or condition associated with the FOXP3 (e.g., IPEX syndrome) can be beneficial for ameliorating one or more symptoms of the disease or condition, for increasing long-term survival, and/or for reducing side effects associated with other treatments. Upon administration of such cells to human subjects, the presence of therapeutic cells that are producing increased levels of functional FOXP3 is beneficial. In some embodiments, effective treatment of a subject gives rise to at least at or about 1%, 3%, 5%, or 7% functional FOXP3 relative to total FOXP3 in the treated subject. In some embodiments, functional FOXP3 is at least at or about 10% of total FOXP3. In some embodiments, functional FOXP3 is at least, at or about, or at most 20%, 30%, 40%, 50%, 60%, 70%, 80%, 90%, or 100% of total FOXP3. Similarly, the introduction of even relatively limited subpopulations of cells having significantly elevated levels of functional FOXP3 can be beneficial in various subjects because in some situations normalized cells will have a selective advantage relative to diseased cells. However, even modest levels of therapeutic cells with elevated levels of functional FOXP3 can be beneficial for ameliorating one or more aspects of the disease or condition in subjects. In some embodiments, at or about 10%, at or about 20%, at or about 30%, at or about 40%, at or about 50%, at or about 60%, at or about 70%, at or about 80%, at or about 90% or more of the therapeutic in subjects to whom such cells are administered are producing increased levels of functional FOXP3.

In embodiments, the delivery of a therapeutic cell composition (e.g., a composition comprising a plurality of cells according to any of the cells described herein) into a subject by a method or route results in at least partial localization of the cell composition at a desired site. A cell composition can be administered by any appropriate route that results in effective treatment in the subject, e.g., administration results in delivery to a desired location in the subject where at least a portion of the composition delivered, e.g., at least 1×10⁴ cells, is delivered to the desired site for a period of time. Modes of administration include injection, infusion, instillation, or ingestion. “Injection” includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, subcapsular, subarachnoid, intraspinal, intracerebrospinal, and intrasternal injection and infusion. In some embodiments, the route is intravenous. For the delivery of cells, administration by injection or infusion can be made.

In one embodiment, the cells are administered systemically, in other words a population of therapeutic cells are administered other than directly into a target site, tissue, or organ, such that it enters, instead, the subject's circulatory system and, thus, is subject to metabolism and other like processes.

The efficacy of a treatment having a composition for the treatment of a disease or condition associated with a FOXP3 (e.g., IPEX syndrome) can be determined by the skilled clinician. However, a treatment is considered effective treatment if any one or all of the signs or symptoms of, as but one example, levels of functional FOXP3 are altered in a beneficial manner (e.g., increased by at least 10%), or other clinically accepted symptoms or markers of disease are improved or ameliorated. Efficacy can also be measured by failure of an individual to worsen as assessed by hospitalization or need for medical interventions (e.g., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing the progression of symptoms; or (2) relieving the disease, e.g., causing regression of symptoms; and (3) preventing or reducing the likelihood of the development of symptoms.

### Compositions

In one aspect, the present disclosure provides compositions for carrying out the methods disclosed herein. A composition can include one or more of the following: a genome-targeting nucleic acid (e.g., a gRNA); a site-directed polypeptide (e.g., a DNA endonuclease) or a nucleotide sequence encoding the site-directed polypeptide; and a polynucleotide to be inserted (e.g., a donor template) to effect the desired genetic modification of the methods disclosed herein.

In some embodiments, a composition has a nucleotide sequence encoding a genome-targeting nucleic acid (e.g., a gRNA).

In some embodiments, a composition has a site-directed polypeptide (e.g. DNA endonuclease). In some embodiments, a composition has a nucleotide sequence encoding the site-directed polypeptide.

In some embodiments, a composition has a polynucleotide (e.g., a donor template) to be inserted into a genome.

In some embodiments, a composition has (i) a nucleotide sequence encoding a genome-targeting nucleic acid (e.g., a gRNA) and (ii) a site-directed polypeptide (e.g., a DNA endonuclease) or a nucleotide sequence encoding the site-directed polypeptide.

In some embodiments, a composition has (i) a nucleotide sequence encoding a genome-targeting nucleic acid (e.g., a gRNA) and (ii) a polynucleotide (e.g., a donor template) to be inserted into a genome.

In some embodiments, a composition has (i) a site-directed polypeptide (e.g., a DNA endonuclease) or a nucleotide sequence encoding the site-directed polypeptide and (ii) a polynucleotide (e.g., a donor template) to be inserted into a genome.

In some embodiments, a composition has (i) a nucleotide sequence encoding a genome-targeting nucleic acid (e.g., a gRNA), (ii) a site-directed polypeptide (e.g., a DNA endonuclease) or a nucleotide sequence encoding the site-directed polypeptide and (iii) a polynucleotide (e.g., a donor template) to be inserted into a genome.

In some embodiments of any of the above compositions, the composition has a single-molecule guide genome-targeting nucleic acid. In some embodiments of any of the above compositions, the composition has a double-molecule genome-targeting nucleic acid. In some embodiments of any of the above compositions, the composition has two or more double-molecule guides or single-molecule guides. In some embodiments, the composition has a vector that encodes the nucleic acid targeting nucleic acid. In some embodiments, the genome-targeting nucleic acid is a DNA endonuclease, in particular, a Cas9.

In some embodiments, a composition can include one or more gRNAs that can be used for genome-edition, in particular, insertion of a sequence encoding a FOXP3 or a derivative thereof into a genome of a cell. The one or more gRNAs can target a genomic site at, within, or near the endogenous FOXP3 gene. Therefore, in some embodiments, the one or more gRNAs can have a spacer sequence complementary to a genomic sequence at, within, or near a FOXP3 gene.

In some embodiments, a gRNA for a composition comprises a spacer sequence selected from any one of SEQ ID NOs: 1-7, 15-20, and 27-29, and variants thereof having at least at or about 50%, at or about 55%, at or about 60%, at or about 65%, at or about 70%, at or about 75%, at or about 80%, at or about 85%, at or about 90% or at or about 95% identity or homology to any one of SEQ ID NOs: 1-7, 15-20, and 27-29. In some embodiments, the variants of gRNA for the kit comprise a spacer sequence having at least at or about 85% homology to any one of SEQ ID NOs: 1-7, 15-20, and 27-29.

In some embodiments, a gRNA for a composition has a spacer sequence that is complementary to a target site in the genome. In some embodiments, the spacer sequence is 15 bases to 20 bases in length. In some embodiments, a complementarity between the spacer sequence to the genomic sequence is at least 80%, at least 85%, at least 90%, at least 95%, at least 96%, at least 97%, at least 98%, at least 99% or at least 100%.

In some embodiments, a composition can have a DNA endonuclease or a nucleic acid encoding the DNA endonuclease and/or a donor template having a nucleic acid sequence encoding a FOXP3 or a functional derivative thereof. In some embodiments, the nucleic acid sequence encoding a FOXP3 or a functional derivative thereof has at least at or about 70% sequence identity, e.g., at least at or about 75%, 80%, 85%, 90%, 92%, 93%, 94%, 95%, 96%, 97%, 98%, 99% or greater sequence identity, to a sequence according to SEQ ID NO: 68. In some embodiments, the DNA endonuclease is a Cas9. In some embodiments, the nucleic acid encoding the DNA endonuclease is DNA or RNA.

In some embodiments, one or more of any nucleic acids for the kit can be encoded in an Adeno Associated Virus (AAV) vector. Therefore, in some embodiments, a gRNA can be encoded in an AAV vector. In some embodiments, a nucleic acid encoding a DNA endonuclease can be encoded in an AAV vector. In some embodiments, a donor template can be encoded in an AAV vector. In some embodiments, two or more nucleic acids can be encoded in a single AAV vector. Thus, in some embodiments, a gRNA sequence and a DNA endonuclease-encoding nucleic acid can be encoded in a single AAV vector.

In some embodiments, a composition can have a liposome or a lipid nanoparticle. Therefore, in some embodiments, any compounds (e.g., a DNA endonuclease or a nucleic acid encoding thereof, gRNA, and donor template) of the composition can be formulated in a liposome or lipid nanoparticle. In some embodiments, one or more such compounds are associated with a liposome or lipid nanoparticle via a covalent bond or non-covalent bond. In some embodiments, any of the compounds can be separately or together contained in a liposome or lipid nanoparticle. Therefore, in some embodiments, each of a DNA endonuclease or a nucleic acid encoding thereof, gRNA, and donor template is separately formulated in a liposome or lipid nanoparticle. In some embodiments, a DNA endonuclease is formulated in a liposome or lipid nanoparticle with gRNA. In some embodiments, a DNA endonuclease or a nucleic acid encoding thereof, gRNA, and donor template are formulated in a liposome or lipid nanoparticle together.

In some embodiments, a composition described above further has one or more additional reagents, where such additional reagents are selected from a buffer, a buffer for introducing a polypeptide or polynucleotide into a cell, a wash buffer, a control reagent, a control vector, a control RNA polynucleotide, a reagent for in vitro production of the polypeptide from DNA, adaptors for sequencing and the like. A buffer can be a stabilization buffer, a reconstituting buffer, a diluting buffer, or the like. In some embodiments, a composition can also include one or more components that can be used to facilitate or enhance the on-target binding or the cleavage of DNA by the endonuclease, or improve the specificity of targeting.

In some embodiments, any components of a composition are formulated with pharmaceutically acceptable excipients such as carriers, solvents, stabilizers, adjuvants, diluents, etc., depending upon the particular mode of administration and dosage form. In embodiments, guide RNA compositions are generally formulated to achieve a physiologically compatible pH, and range from a pH of at or about 3 to a pH of at or about 11, at or about pH 3 to at or about pH 7, depending on the formulation and route of administration. In some embodiments, the pH is adjusted to a range from at or about pH 5.0 to at or about pH 8. In some embodiments, the composition has a therapeutically effective amount of at least one compound as described herein, together with one or more pharmaceutically acceptable excipients. Optionally, the composition can have a combination of the compounds described herein, or can include a second active ingredient useful in the treatment or prevention of bacterial growth (for example and without limitation, anti-bacterial or anti-microbial agents), or can include a combination of reagents of the disclosure. In some embodiments, gRNAs are formulated with other one or more nucleic acids, e.g., nucleic acid encoding a DNA endonuclease and/or a donor template. Alternatively, a nucleic acid encoding a DNA endonuclease and a donor template, separately or in combination with other nucleic acids, are formulated with the method described above for gRNA formulation.

Suitable excipients can include, for example, carrier molecules that include large, slowly metabolized macromolecules such as proteins, polysaccharides, polylactic acids, polyglycolic acids, polymeric amino acids, amino acid copolymers, and inactive virus particles. Other exemplary excipients include antioxidants (for example and without limitation, ascorbic acid), chelating agents (for example and without limitation, EDTA), carbohydrates (for example and without limitation, dextrin, hydroxyalkylcellulose, and hydroxyalkylmethylcellulose), stearic acid, liquids (for example and without limitation, oils, water, saline, glycerol, and ethanol), wetting or emulsifying agents, pH buffering substances, and the like.

In some embodiments, any compounds (e.g., a DNA endonuclease or a nucleic acid encoding thereof, gRNA, and donor template) of a composition can be delivered into a cell via transfection, such as chemical transfection (e.g., lipofection) or electroporation. In some embodiments, a DNA endonuclease can be pre-complexed with a gRNA, forming a ribonucleoprotein (RNP) complex, prior to the provision to the cell. In some embodiments, the RNP complex is delivered into the cell via transfection. In such embodiments, the donor template is delivered into the cell via transfection.

In some embodiments, a composition refers to a therapeutic composition having therapeutic cells that are used in an ex vivo treatment method.

In embodiments, therapeutic compositions contain a physiologically tolerable carrier together with the cell composition, and optionally at least one additional bioactive agent as described herein, dissolved or dispersed therein as an active ingredient. In some embodiments, the therapeutic composition is not substantially immunogenic when administered to a mammal or human subject for therapeutic purposes, unless so desired.

In general, the genetically modified, therapeutic cells described herein are administered as a suspension with a pharmaceutically acceptable carrier. One of skill in the art will recognize that a pharmaceutically acceptable carrier to be used in a cell composition will not include buffers, compounds, cryopreservation agents, preservatives, or other agents in amounts that substantially interfere with the viability of the cells to be delivered to the subject. A formulation having cells can include e.g., osmotic buffers that permit cell membrane integrity to be maintained, and optionally, nutrients to maintain cell viability or enhance engraftment upon administration. Such formulations and suspensions are known to those of skill in the art and/or can be adapted for use with the progenitor cells, as described herein, using routine experimentation.

In some embodiments, a cell composition can also be emulsified or presented as a liposome composition, provided that the emulsification procedure does not adversely affect cell viability. The cells and any other active ingredient can be mixed with one or more excipients that are pharmaceutically acceptable and compatible with the active ingredient, and in amounts suitable for use in the therapeutic methods described herein.

Additional agents included in a cell composition can include pharmaceutically acceptable salts of the components therein. Pharmaceutically acceptable salts include the acid addition salts (formed with the free amino groups of the polypeptide) that are formed with inorganic acids, such as, for example, hydrochloric or phosphoric acids, or such organic acids as acetic, tartaric, mandelic, and the like. Salts formed with the free carboxyl groups can also be derived from inorganic bases, such as, for example, sodium, potassium, ammonium, calcium, or ferric hydroxides, and such organic bases as isopropylamine, trimethylamine, 2-ethylamino ethanol, histidine, procaine, and the like.

Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active compound used in the cell compositions that is effective in the treatment of a particular disorder or condition will depend on the nature of the disorder or condition, and can be determined by known clinical techniques.

In some embodiments, the cells, such as mammalian cells, include the protein sequences as described in the embodiments herein. In some embodiments, the compositions include CD4+ T cells that have a CISC comprising an extracellular binding domain, a hinge domain, a transmembrane domain, and signaling domain. In some embodiments, the CISC is an IL2R-CISC. In some embodiments, the composition further comprises a cell, such as a mammalian cell, preparation comprising CD8+ T cells that have a CISC comprising an extracellular binding domain, a hinge domain, a transmembrane domain, and a signaling domain. In some embodiments, the CISC components dimerize in the presence of a ligand, preferably simultaneously. In some embodiments, each of these populations can be combined with one another or other cell types to provide a composition.

In some embodiments, the cells of the composition are CD4+ cells. The CD4+ cell can be T helper lymphocyte cells, naïve CD4+ T cells, central memory CD4+ T cells, effector memory CD4+ T cells, or bulk CD4+ T cells. In some embodiments, the CD4+ helper lymphocyte cell is a naïve CD4+ T cell, wherein the naïve CD4+ T cell comprises a CD45RO−, CD45RA+, and/or is a CD62L+CD4+ T cell.

In some embodiments, the cells of the composition are CD8+ cells. The CD8+ cell can be a T cytotoxic lymphocyte cell, a naïve CD8+ T cell, central memory CD8+ T cell, effector memory CD8+ T cell and/or bulk CD8+ T cell. In some embodiments, the CD8+ cytotoxic T lymphocyte cell is a central memory T cell, wherein the central memory T cell comprises a CD45RO+, CD62L+, and/or CD8+ T cell. In some embodiments, the CD8+ cytotoxic T lymphocyte cell is a central memory T cell and the CD4+ helper T lymphocyte cell is a naïve or central memory CD4+ T cell.

In some embodiments, the compositions comprise T cell precursors. In some embodiments, the compositions comprise hematopoietic stem cells. In some embodiments, the composition comprises a host cell wherein the host cell is a CD8+T cytotoxic lymphocyte cell selected from the group consisting of naïve CD8+ T cells, central memory CD8+ T cells, effector memory CD8+ T cells and bulk CD8+ T cells or a CD4+T helper lymphocyte cell that is selected from the group consisting of naïve CD4+ T cells, central memory CD4+ T cells, effector memory CD4+ T cells, and bulk CD4+ T cells and a second host cell, wherein the second host cell is a precursor T cell. In some embodiments, the precursor T cell is a hematopoietic stem cell.

In some compositions, the cells are NK cells.

In some embodiments, the cell is CD8+ or a CD4+ cell. In some embodiments, the cell is a CD8+T cytotoxic lymphocyte cell selected from the group consisting of naïve CD8+ T cells, central memory CD8+ T cells, effector memory CD8+ T cells and bulk CD8+ T cells. In some embodiments, the cell is a CD4+T helper lymphocyte cell that is selected from the group consisting of naïve CD4+ T cells, central memory CD4+ T cells, effector memory CD4+ T cells, and bulk CD4+ T cells. In some embodiments, the cell is a precursor T cell. In some embodiments, the cell is a stem cell. In some embodiments, the cell is a hematopoietic stem cell or NK cell. In some embodiments, the cell is a B cell. In some embodiments, the cell is a neuronal stem cell. In some embodiments, the cell further comprises a chimeric antigen receptor.

### Kits

Some embodiments provide a kit that contains any of the above-described compositions, e.g., a composition for genome edition or a cell composition (e.g., a therapeutic cell composition), and one or more additional components.

In some embodiments, kits and systems including the cells, expression vectors, and protein sequences are provided and described herein. Thus, for example, provided herein is a kit comprising one or more of: a protein sequence as described herein; an expression vector as described herein; and/or a cell as described herein. Also provided is a system for selectively activation a signal into an interior of a cell, the system comprising a cell as described herein, wherein the cell comprises an expression vector as described herein comprising a nucleic acid encoding a protein sequence as described herein.

In some embodiments, a kit can have one or more additional therapeutic agents that can be administered simultaneously or in sequence with the composition for a desired purpose, e.g., genome edition or cell therapy.

In some embodiments, a kit can further include instructions for using the components of the kit to practice the methods. The instructions for practicing the methods are generally recorded on a suitable recording medium. For example, the instructions can be printed on a substrate, such as paper or plastic, etc. The instructions can be present in the kits as a package insert, in the labeling of the container of the kit or components thereof (such as associated with the packaging or subpackaging), etc. The instructions can be present as an electronic storage data file present on a suitable computer readable storage medium, e.g. CD-ROM, diskette, flash drive, etc. In some instances, the actual instructions are not present in the kit, but means for obtaining the instructions from a remote source (e.g., via the internet), can be provided. An example of this embodiment is a kit that includes a web address where the instructions can be viewed and/or from which the instructions can be downloaded. As with the instructions, this means for obtaining the instructions can be recorded on a suitable substrate.

### Exemplary Embodiments

In some embodiments, a method of making a genetically engineered cell is provided, wherein the method comprises: providing a cell, wherein the cell comprises a first nucleic acid comprising at least one targeted locus; providing a CAS9 protein or a second nucleic acid encoding a CAS9 protein; introducing the CAS9 protein or the second nucleic acid into the cell; introducing a third nucleic acid encoding at least one CRISPR guide sequence or a set of nucleic acids encoding at least one CRISPR guide sequence, wherein the at least one CRISPR guide sequence is configured to hybridize to the at least one targeted locus; and introducing a fourth nucleic acid into the cell, wherein the fourth nucleic acid comprises a gene delivery cassette. In some embodiments, the method further comprises activating the cell, wherein the activating is performed before the introducing of the second nucleic acid into the cell. In some embodiments, the activating is performed by contacting the cell with CD3 and/or CD28. In some embodiments, the at least one targeted locus is a FOXP3 locus, AAVS1 locus or a TCRa (TRAC) locus. In some embodiments, the second nucleic acid, third nucleic acid, the set of nucleic acids and/or the fourth nucleic acid is provided in one or more vectors. In some embodiments, the one or more vectors is a viral vector. In some embodiments, the viral vector is an Adeno-associated virus (AAV) vector. In some embodiments, the AAV vector is a self-complementary vector. In some embodiments, the AAV vector is a single stranded vector. In some embodiments, the AAV vector is a combination of a self-complementary vector and a single stranded vector. In some embodiments, the second nucleic acid encoding the CAS9 protein is an mRNA. In some embodiments, the at least one guide sequence comprises a sequence set forth in any one of SEQ ID NOs: 1-7, 15-20, 27-29, 33 and/or 34. In some embodiments, the second nucleic acid, the third nucleic acid, the set of nucleic acids and/or the fourth nucleic acid are codon optimized for expression in a eukaryotic cell, such as a human cell. In some embodiments, the fourth nucleic acid comprises a sequence encoding a human codon optimized FOXP3 cDNA sequence. In some embodiments, the fourth nucleic acid sequence comprises a sequence set forth in SEQ ID NO: 68 or 69. In some embodiments, the fourth nucleic acid further comprises a promoter. In some embodiments, the promoter is a MND promoter, PGK promoter or an E2F promoter. In some embodiments, the fourth nucleic acid further comprises a sequence encoding a low affinity nerve growth factor receptor coding sequence (LNGFR), μCISC, CISCγ, FRB and/or LNGFRe (LNGFR epitope coding sequence). In some embodiments, the method further comprises introducing a fifth nucleic into the cell, wherein the fifth nucleic acid comprises a second gene delivery cassette. In some embodiments, the fifth nucleic acid is provided in a vector. In some embodiments, the vector is an AAV vector. In some embodiments, the fifth nucleic acid comprises a sequence encoding CISC, FRB, a marker protein, μCISC, and/or βCISC. In some embodiments, the fourth and or fifth nucleic acid further comprises a sequence encoding a P2A self-cleaving peptide, e.g., a sequence according to SEQ ID NO: 89. In some embodiments, the fourth and or fifth sequence further comprises a sequence encoding a polyA sequence. In some embodiments, the polyA sequence comprises a SV40polyA or 3′UTR of FOXP3. In some embodiments, the fourth sequence comprises a sequence as set forth in any one of SEQ ID NO: 37-42. In some embodiments, a fourth a fifth nucleic acid are introduced into the cell, wherein the fourth and fifth nucleic acid comprises a sequence as set forth in SEQ ID NO: 37 and 43, SEQ ID NO: 37 and 44, SEQ ID NO: 38 and 43, SEQ ID NO: 38 and 44, SEQ ID NO: 45 and 46, or SEQ ID NO: 45 and 47, respectively. In some embodiments, the cell is a primary human lymphocyte. In some embodiments, the fourth nucleic acid comprises at least one homology arm with a locus specific sequence and, wherein the homology arm length is configured for efficient packaging into an AAV vector. In some embodiments, the at least one homology arm comprises a length of 0.25, 0.3, 0.45, 0.6 or 0.8 kb or any length in between a range defined by any two aforementioned values. In some embodiments, the marker is LNGF, RQR8 or EGFRt. In some embodiments, the method further comprises introducing into the cell a sixth nucleic acid encoding a protein or cytokine for co-expression with FOXP3. In some embodiments, the protein of cytokine is a T cell receptor, a chimeric antigen receptor or IL-10. In some embodiments, the method further comprises selecting the cells by enrichment of the marker.

In some embodiments, a cell for expression of FOXP3 is provided, manufactured by the method of any one of the embodiments herein. In some embodiments, the method comprises providing a cell, wherein the cell comprises a first nucleic acid comprising at least one targeted locus; providing a CAS9 protein or a second nucleic acid encoding a CAS9 protein; introducing the CAS9 protein or the second nucleic acid into the cell; introducing a third nucleic acid encoding at least one CRISPR guide sequence or a set of nucleic acids encoding at least one CRISPR guide sequence, wherein the at least one CRISPR guide sequence is configured to hybridize to the at least one targeted locus; and introducing a fourth nucleic acid into the cell, wherein the fourth nucleic acid comprises a gene delivery cassette. In some embodiments, the method further comprises activating the cell, wherein the activating is performed before the introducing of the second nucleic acid into the cell. In some embodiments, the activating is performed by contacting the cell with CD3 and/or CD28. In some embodiments, the at least one targeted locus is a FOXP3 locus, AAVS1 locus or a TCRa (TRAC) locus. In some embodiments, the second nucleic acid, third nucleic acid, the set of nucleic acids and/or the fourth nucleic acid is provided in one or more vectors. In some embodiments, the one or more vectors is a viral vector. In some embodiments, the viral vector is an Adeno-associated virus (AAV) vector. In some embodiments, the AAV vector is a self-complementary vector. In some embodiments, the AAV vector is a single stranded vector. In some embodiments, the AAV vector is a combination of a self-complementary vector and a single stranded vector. In some embodiments, the second nucleic acid encoding the CAS9 protein is an mRNA. In some embodiments, the at least one guide sequence comprises a sequence set forth in any one of SEQ ID NOs: 1-7, 15-20, 27-29, 33 and/or 34. In some embodiments, the second nucleic acid, the third nucleic acid, the set of nucleic acids and/or the fourth nucleic acid are codon optimized for expression in a eukaryotic cell, such as a human cell. In some embodiments, the fourth nucleic acid comprises a sequence encoding a human codon optimized FOXP3 cDNA sequence. In some embodiments, the fourth nucleic acid sequence comprises a sequence set forth in SEQ ID NO: 68 or 69. In some embodiments, the fourth nucleic acid further comprises a promoter. In some embodiments, the promoter is a MND promoter, PGK promoter or an E2F promoter. In some embodiments, the fourth nucleic acid further comprises a sequence encoding a low affinity nerve growth factor receptor coding sequence (LNGFR), μCISC, CISCγ, FRB and/or LNGFRe (LNGFR epitope coding sequence). In some embodiments, the method further comprises introducing a fifth nucleic into the cell, wherein the fifth nucleic acid comprises a second gene delivery cassette. In some embodiments, the fifth nucleic acid is provided in a vector. In some embodiments, the vector is an AAV vector. In some embodiments, the fifth nucleic acid comprises a sequence encoding CISC, FRB, a marker protein, μCISC, and/or βCISC. In some embodiments, the fourth and or fifth nucleic acid further comprises a sequence encoding a P2A self-cleaving peptide, e.g., a sequence according to SEQ ID NO: 89. In some embodiments, the fourth and or fifth sequence further comprises a sequence encoding a polyA sequence. In some embodiments, the polyA sequence comprises a SV40polyA or 3′UTR of FOXP3. In some embodiments, the fourth sequence comprises a sequence as set forth in any one of SEQ ID NO: 37-42. In some embodiments, a fourth a fifth nucleic acid are introduced into the cell, wherein the fourth and fifth nucleic acid comprises a sequence as set forth in SEQ ID NO: 37 and 43, SEQ ID NO: 37 and 44, SEQ ID NO: 38 and 43, SEQ ID NO: 38 and 44, SEQ ID NO: 45 and 46, or SEQ ID NO: 45 and 47, respectively. In some embodiments, the cell is a primary human lymphocyte. In some embodiments, the fourth nucleic acid comprises at least one homology arm with a locus specific sequence and, wherein the homology arm length is configured for efficient packaging into an AAV vector. In some embodiments, the at least one homology arm comprises a length of 0.25, 0.3, 0.45, 0.6 or 0.8 kb or any length in between a range defined by any two aforementioned values. In some embodiments, the marker is LNGF, RQR8 or EGFRt. In some embodiments, the method further comprises introducing into the cell a sixth nucleic acid encoding a protein or cytokine for co-expression with FOXP3. In some embodiments, the protein of cytokine is a T cell receptor, a chimeric antigen receptor or IL-10. In some embodiments, the method further comprises selecting the cells by enrichment of the marker. In some embodiments, the cell is a primary human lymphocyte. In some embodiments, FOXP3 is expressed constitutively or the expression is regulated.

In some embodiments, a cell for expression of FOXP3 is provided, the cell comprising: a nucleic acid encoding a gene encoding a FOXP3. In some embodiments, the gene encoding a FOXP3 is integrated at a FOXP3 or a non-FOXP3 locus. In some embodiments, the non-FOXP3 locus is an AAVS1 locus or a TCRa (TRAC) locus. In some embodiments, the cell is a primary human lymphocyte. In some embodiments, the cell expresses CISCβ: FRB-IL2Rβ, DISC, CISC-FRB, μDISC, μCISC-FRB, FRB, LNGFR and/or LNGFRe. In some embodiments, the cell comprises a T_{reg} phenotype.

In some embodiments, a composition comprising the cell of any one of the embodiments herein is provided. In some embodiments, the cell is manufactured by the method of any one of the embodiments herein. In some embodiments, the method comprises providing a cell, wherein the cell comprises a first nucleic acid comprising at least one targeted locus; providing a CAS9 protein or a second nucleic acid encoding a CAS9 protein; introducing the CAS9 protein or the second nucleic acid into the cell; introducing a third nucleic acid encoding at least one CRISPR guide sequence or a set of nucleic acids encoding at least one CRISPR guide sequence, wherein the at least one CRISPR guide sequence is configured to hybridize to the at least one targeted locus; and introducing a fourth nucleic acid into the cell, wherein the fourth nucleic acid comprises a gene delivery cassette. In some embodiments, the method further comprises activating the cell, wherein the activating is performed before the introducing of the second nucleic acid into the cell. In some embodiments, the activating is performed by contacting the cell with CD3 and/or CD28. In some embodiments, the at least one targeted locus is a FOXP3 locus, AAVS1 locus or a TCRa (TRAC) locus. In some embodiments, the second nucleic acid, third nucleic acid, the set of nucleic acids and/or the fourth nucleic acid is provided in one or more vectors. In some embodiments, the one or more vectors is a viral vector. In some embodiments, the viral vector is an Adeno-associated virus (AAV) vector. In some embodiments, the AAV vector is a self-complementary vector. In some embodiments, the AAV vector is a single stranded vector. In some embodiments, the AAV vector is a combination of a self-complementary vector and a single stranded vector. In some embodiments, the second nucleic acid encoding the CAS9 protein is an mRNA. In some embodiments, the at least one guide sequence comprises a sequence set forth in any one of SEQ ID NOs: 1-7, 15-20, 27-29, 33 and/or 34. In some embodiments, the second nucleic acid, the third nucleic acid, the set of nucleic acids and/or the fourth nucleic acid are codon optimized for expression in a eukaryotic cell, such as a human cell. In some embodiments, the fourth nucleic acid comprises a sequence encoding a human codon optimized FOXP3 cDNA sequence. In some embodiments, the fourth nucleic acid sequence comprises a sequence set forth in SEQ ID NO: 68 or 69. In some embodiments, the fourth nucleic acid further comprises a promoter. In some embodiments, the promoter is a MND promoter, PGK promoter or an E2F promoter. In some embodiments, the fourth nucleic acid further comprises a sequence encoding a low affinity nerve growth factor receptor coding sequence (LNGFR), μCISC, CISCγ, FRB and/or LNGFRe (LNGFR epitope coding sequence). In some embodiments, the method further comprises introducing a fifth nucleic into the cell, wherein the fifth nucleic acid comprises a second gene delivery cassette. In some embodiments, the fifth nucleic acid is provided in a vector. In some embodiments, the vector is an AAV vector. In some embodiments, the fifth nucleic acid comprises a sequence encoding CISC, FRB, a marker protein, μCISC, and/or βCISC. In some embodiments, the fourth and or fifth nucleic acid further comprises a sequence encoding a P2A self-cleaving peptide, e.g., a sequence according to SEQ ID NO: 89. In some embodiments, the fourth and or fifth sequence further comprises a sequence encoding a polyA sequence. In some embodiments, the polyA sequence comprises a SV40polyA or 3′UTR of FOXP3. In some embodiments, the fourth sequence comprises a sequence as set forth in any one of SEQ ID NO: 37-42. In some embodiments, a fourth a fifth nucleic acid are introduced into the cell, wherein the fourth and fifth nucleic acid comprises a sequence as set forth in SEQ ID NO: 37 and 43, SEQ ID NO: 37 and 44, SEQ ID NO: 38 and 43, SEQ ID NO: 38 and 44, SEQ ID NO: 45 and 46, or SEQ ID NO: 45 and 47, respectively. In some embodiments, the cell is a primary human lymphocyte. In some embodiments, the fourth nucleic acid comprises at least one homology arm with a locus specific sequence and, wherein the homology arm length is configured for efficient packaging into an AAV vector. In some embodiments, the at least one homology arm comprises a length of 0.25, 0.3, 0.45, 0.6 or 0.8 kb or any length in between a range defined by any two aforementioned values. In some embodiments, the marker is LNGF, RQR8 or EGFRt. In some embodiments, the method further comprises introducing into the cell a sixth nucleic acid encoding a protein or cytokine for co-expression with FOXP3. In some embodiments, the protein of cytokine is a T cell receptor, a chimeric antigen receptor or IL-10. In some embodiments, the method further comprises selecting the cells by enrichment of the marker. In some embodiments, the cell is a primary human lymphocyte. In some embodiments, FOXP3 is expressed constitutively or the expression is regulated. In some embodiments, the cell comprises a nucleic acid encoding a gene encoding a FOXP3. In some embodiments, the gene encoding a FOXP3 is integrated at a FOXP3 or a non-FOXP3 locus. In some embodiments, the non-FOXP3 locus is an AAVS1 locus or a TCRa (TRAC) locus. In some embodiments, the cell is a primary human lymphocyte. In some embodiments, the cell expresses CISCβ: FRB-IL2Rβ, DISC, CISC-FRB, μDISC, μCISC-FRB, FRB, LNGFR and/or LNGFRe. In some embodiments, the cell comprises a T_{reg} phenotype.

In some embodiments, a method for treating, ameliorating, and/or inhibiting a disease and/or a condition in a subject is provided, the method comprising: providing to a subject having a disease and/or a condition the cell or the composition of any of the embodiments herein. In some embodiments, the cell is manufactured by the method of any one of the embodiments herein. In some embodiments, the method comprises providing a cell, wherein the cell comprises a first nucleic acid comprising at least one targeted locus; providing a CAS9 protein or a second nucleic acid encoding a CAS9 protein; introducing the CAS9 protein or the second nucleic acid into the cell; introducing a third nucleic acid encoding at least one CRISPR guide sequence or a set of nucleic acids encoding at least one CRISPR guide sequence, wherein the at least one CRISPR guide sequence is configured to hybridize to the at least one targeted locus; and introducing a fourth nucleic acid into the cell, wherein the fourth nucleic acid comprises a gene delivery cassette. In some embodiments, the method further comprises activating the cell, wherein the activating is performed before the introducing of the second nucleic acid into the cell. In some embodiments, the activating is performed by contacting the cell with CD3 and/or CD28. In some embodiments, the at least one targeted locus is a FOXP3 locus, AAVS1 locus or a TCRa (TRAC) locus. In some embodiments, the second nucleic acid, third nucleic acid, the set of nucleic acids and/or the fourth nucleic acid is provided in one or more vectors. In some embodiments, the one or more vectors is a viral vector. In some embodiments, the viral vector is an Adeno-associated virus (AAV) vector. In some embodiments, the AAV vector is a self-complementary vector. In some embodiments, the AAV vector is a single stranded vector. In some embodiments, the AAV vector is a combination of a self-complementary vector and a single stranded vector. In some embodiments, the second nucleic acid encoding the CAS9 protein is an mRNA. In some embodiments, the at least one guide sequence comprises a sequence set forth in any one of SEQ ID NOs: 1-7, 15-20, 27-29, 33 and/or 34. In some embodiments, the second nucleic acid, the third nucleic acid, the set of nucleic acids and/or the fourth nucleic acid are codon optimized for expression in a eukaryotic cell, such as a human cell. In some embodiments, the fourth nucleic acid comprises a sequence encoding a human codon optimized FOXP3 cDNA sequence. In some embodiments, the fourth nucleic acid sequence comprises a sequence set forth in SEQ ID NO: 68 or 69. In some embodiments, the fourth nucleic acid further comprises a promoter. In some embodiments, the promoter is a MND promoter, PGK promoter or an E2F promoter. In some embodiments, the fourth nucleic acid further comprises a sequence encoding a low affinity nerve growth factor receptor coding sequence (LNGFR), nCISC, CISCγ, FRB and/or LNGFRe (LNGFR epitope coding sequence). In some embodiments, the method further comprises introducing a fifth nucleic into the cell, wherein the fifth nucleic acid comprises a second gene delivery cassette. In some embodiments, the fifth nucleic acid is provided in a vector. In some embodiments, the vector is an AAV vector. In some embodiments, the fifth nucleic acid comprises a sequence encoding CISC, FRB, a marker protein, μCISC, and/or βCISC. In some embodiments, the fourth and or fifth nucleic acid further comprises a sequence encoding a P2A self-cleaving peptide, e.g., a sequence according to SEQ ID NO: 89. In some embodiments, the fourth and or fifth sequence further comprises a sequence encoding a polyA sequence. In some embodiments, the polyA sequence comprises a SV40polyA or 3′UTR of FOXP3. In some embodiments, the fourth sequence comprises a sequence as set forth in any one of SEQ ID NO: 37-42. In some embodiments, a fourth a fifth nucleic acid are introduced into the cell, wherein the fourth and fifth nucleic acid comprises a sequence as set forth in SEQ ID NO: 37 and 43, SEQ ID NO: 37 and 44, SEQ ID NO: 38 and 43, SEQ ID NO: 38 and 44, SEQ ID NO: 45 and 46, or SEQ ID NO: 45 and 47, respectively. In some embodiments, the cell is a primary human lymphocyte. In some embodiments, the fourth nucleic acid comprises at least one homology arm with a locus specific sequence and, wherein the homology arm length is configured for efficient packaging into an AAV vector. In some embodiments, the at least one homology arm comprises a length of 0.25, 0.3, 0.45, 0.6 or 0.8 kb or any length in between a range defined by any two aforementioned values. In some embodiments, the marker is LNGF, RQR8 or EGFRt. In some embodiments, the method further comprises introducing into the cell a sixth nucleic acid encoding a protein or cytokine for co-expression with FOXP3. In some embodiments, the protein of cytokine is a T cell receptor, a chimeric antigen receptor or IL-10. In some embodiments, the method further comprises selecting the cells by enrichment of the marker. In some embodiments, the cell is a primary human lymphocyte. In some embodiments, FOXP3 is expressed constitutively or the expression is regulated. In some embodiments, the disease is an autoimmune disease. In some embodiments, the disease is X-linked (IPEX) syndrome. In some embodiments, the condition is Graft-versus Host Disease (GVHD). In some embodiments, the subject has a solid organ transplant.

Some embodiments include a medicament for use in treating, ameliorating, and/or inhibiting a disease and/or a condition in a subject. More embodiments concern a genetically modified cells in which the genome of the cell is edited by one of the methods described herein for use in inhibiting or treating a disease or condition associated with FOXP3, such as an inflammatory disease or an autoimmune disease. Additional embodiments concern use of a genetically modified cells in which the genome of the cell is edited by any one of the methods herein as a medicament.

In some embodiments, the cell is not a germ cell.

### EXAMPLES

### Example 1: Expression of Endogenous FOXP3 from Healthy Donors but not IPEX Donors Acquires Suppressive Function In Vitro

This experiment demonstrates that providing a constitutive promoter for FOXP3 results in suppressive function in CD4+T_{conv} cells only if FOXP3 is functional. Cells from IPEX patients were engineered using TALEN mRNA and AAV donor template containing MND-GFP flanked by FOXP3 homology arms. This gene editing approach resulted in the introduction of the MND promoter and GFP coding sequence at the FOXP3 locus with GFP coding sequence in-frame with the endogenous FOXP3 coding sequence.

The constitutive MND promoter in the engineered cells expressed GFP infused with the endogenous FOXP3 with down-stream mutations. Due to the loss-of-function mutation of FOXP3, knocking in a constitutive promoter upstream of FOXP3 gene failed to acquire CD4+T_{conv} cells suppressive function. Expression of functional FOXP3 cDNA was required to acquire suppressive function.

Cells were assayed using a FACS assay. The cells used for the test included T cells that expressed endogenous FOXP3 from a healthy donor and two donors suffering from IPEX. As shown in the table below, flow cytometry of T_{eff} cells and mock treatment (“T_{eff}+mock”) showed a reduced percentage of cells expressing endogenous FOXP3 as compared to after editing T cells to express endogenous FOXP3 (“T_{eff}+edT_{reg}”). While in each case, the endogenous FOXP3 expression increased, only in the healthy subject did T_{eff} function decrease.

| | % Endogenous FOXP3+ cells | | Reduction in T_{eff} |
|---|---|---|---|
| Sample | T_{eff} + mock | T_{eff} + edT_{reg} | function with edT_{reg}? |
| Healthy control | 39 | 64 | Yes |
| IPEX subject #1 | 28 | 42 | No |
| IPEX subject #2 | 32 | 57 | No |

The edT_{reg} cells generated separately from T cells originating from IPEX subjects with down-stream mutations in the FOXP3 gene expressed GFP due to expression of the mutated, non-functional FOXP3 protein, but did not suppress T_{eff} proliferation, in contrast to the edT_{reg} cells generated from the healthy donor T cells. This indicates that restoration of FOXP3 activity is also required for treatment of IPEX.

### Example 2: Generation of FOXP3-Expressing Engineered Regulatory T Cells

FOXP3-expressing engineered regulatory T cells were generated via gene editing using CRISPR/Cas9-sgRNA RNP and AAV-delivered donor templates that offer promise for treatment and suppression of Graft-versus Host Disease (GVHD) and autoimmune diseases. Regulatory T cells were obtained from subjects for gene editing. AAV vectors were used to deliver donor templates for treatment and suppression of Graft versus Host Disease (GVHD) and autoimmune disease. The targeted locus was selected from the locus for FOXP3 (single AAV construct), AAVS1 (single or dual AAV constructs), and TCR (single or dual AAV constructs). AAV donor template constructs were used to engineer T_{reg} with a single AAV template (Constructs A, B, C, D and F in the table below). AAV donor template constructs were also used to engineer T_{reg} with a dual AAV templates (see Constructs A+G, A+H, B+G, B+H, I+J, and I+K).

| | Expression cassettes in | |
|---|---|---|
| ID | AAV donor template | Construct diagram |
| A | FOXP3cDNA-LNGFR | ITR-HA-MND-FOXP3cDNA-2A-LNGFR-pA-HA-ITR |
| B | LNGFR-FOXP3cDNA | ITR-HA-MND- LNGFR-2A-FOXP3cDNA-pA-HA-ITR |
| C | FOXP3cDNA-μDISC | ITR-HA-MND-FOXP3cDNA-2A-μDISC-2A-FRB-pA- |
| | | HA-ITR |
| D | FOXP3cDNA- | ITR-HA-MND-FOXP3cDNA-2A-LNGFRe-2A-μDISC- |
| | LNGFRe-μDISC | 2A-FRB-pA-HA-ITR |
| E | pDISC-FOXP3cDNA | ITR-HA-MND-μDISC-2A-FRB-2A-FOXP3cDNA-pA- |
| | | HA-ITR |
| F | LNGFRe-μDISC- | ITR-HA-MND-LNGFRe-2A-μDISC-2A-FRB-2A- |
| | FOXP3cDNA | FOXP3cDNA-pA-HA-ITR |
| G | DISC | ITR-HA-MND-DISC-2A-FRB-2A-marker-pA-HA-ITR |
| H | μDISC | ITR-HA-MND-μDISC-2A-FRB-marker-pA-HA-ITR |
| I | CISCβ-DN | ITR-HA-MND-CISCβ-2A-FRB-2A-marker-pA-HA-ITR |
| J | CISCγ-FOXP3cDNA- | ITR-HA-MND-CISCγ-2A-FOXP3cDNA-2A-LNGFR- |
| | LNGFR | pA-HA-ITR |
| K | CISCγ-LNGFR- | ITR-HA-MND-CISCγ-2A-LNGFR-2A-FOXP3cDNA- |
| | FOXP3cDNA | pA-HA-ITR |

In the foregoing table, FOXP3cDNA is a nucleic acid sequence, such as a codon-optimized sequence, encoding expression of a FOXP3 mRNA; CISCβ is FRB-IL2Rβ; CISCγ is FKBP-IL2Rγ; DISC is CISC-FRB; μDISC is μCISC-FRB; FRB is expressed intracellularly to function as a decoy for rapamycin; LNGFR is a low affinity nerve growth factor receptor coding sequenc; LNGFRe is an LNGFR epitope coding sequence; and 2A represents a nucleic acid encoding P2A self-cleaving peptide.

Construct variants included locus-specific homology arm sequences with varying lengths (e.g., 0.25, 0.3, 0.45, 0.6, or 0.8 kb), selection markers such as LNGFR, RQR8, or EGFRt, promoters such as MND, PGK, or E2F, and polyA (pA) sequence such as an SV40polyA sequence or 3′UTR of a FOXP3.

On-Target and Off-Target Cutting Efficiency of RNP Targeting Human FOXP3

CRISPR-Cas9/sgRNA RNP comprised novel spacer sequences. The spacer sequences T1, T3, T4, T7, T9, and T18, were designed to target human FOXP3 locus in exon 1. To perform on-target and off-target cutting analysis, genomic DNA was extracted from CD4+ T cells transfected with CRISPR-Cas9/gRNA RNP comprising a spacer sequence as described herein. Genomic DNA from mock-transfected CD4+ T cells was also extracted as a reference control.

The on-target cutting efficiency was determined by colony sequencing and presented as % MET (Non-homologous end joining). High % MET indicated high cutting efficiency. Briefly, forward and reverse PCR primers were designed approximately 250 to 300 bp upstream and downstream of the cut site. PCR reactions were set up using the designed primer pair to amplify DNA fragments from the genomic DNA. PCR amplicons were resolved on agarose gel, extracted, and subjected to pJET PCR cloning. The resulting bacteria colonies were used for direct colony sequencing to obtain sequences of the cloned PCR fragments. All sequencing reads were compared with reference sequence to determine the presence of insertion or deletion due to NHEJ of DNA double strand breaks. The percentage of clones that had NHEJ was calculated.

Shown in the table below is the percentage of successful non-homologous end joining following treatment with the CRISPR-CAS9/gRNA system with the guides sequences for the FOXP3 locus T1, T3, T4, T7, T9 and T18. The RNPs comprising spacer sequences T1, T3, T4, T7, T9, and T18 targeting human FOXP3 locus have a high on-target cutting efficiency, of from 71% to 100%. In particular, the RNPs comprising T3, T4, T7, T9, and T18 exhibited about 90%-100% on-target cutting efficiency. As shown in the table below, the RNPs comprising the guides targeting human FOXP3 locus have high cutting efficiency and the proteins were shown to be expressed after the donor nucleic acid was integrated into the locus.

| RNP Cas9/gRNA | |
|---|---|
| with indicated spacer sequence | % NHEJ |
| T1 | 71 |
| T3 | 100 |
| T4 | 90 |
| T7 | 100 |
| T9 | 89 |
| T18 | 91 |

Off target analysis (OTA) of CRISPR-Cas9/gRNA RNP comprising T3, T4, T9 and T18 spacer sequences was determined. For each guide, the top 5 to 7 off-targets predicted by CRISPR-Cas9 target online predictor (CCTop) were analyzed for the presence of indels (insertions or deletions). PCR primer pairs for each target were designed using a similar strategy used for on-target analysis. After PCR amplification and purification, the amplicons were subjected to sequencing reactions. Sequencing reads were analyzed by Tracking Indels by DEcomposition(TIDE) or Inference of CRISPR Edits (ICE).

RNPs comprising Cas9/gRNA having T3 or T9 spacer sequence exhibited 4% or less cutting efficiency on predicted off-target cutting sites (for T3: DACT2, SLC2A6, FOXA1, EXTL1, CFAPa9, or intergenic region on chr10; for T9: PPP2R3B, TMCO4, RND1, chr11: 11θr, THNCL1, or COL5A1).

On-Target Cutting Efficiency of RNPs Targeting Human AAVS1

Human CD4+ T cells from healthy donors were then used for assaying the on-target cutting efficiency of RNPs comprising Cas9/gRNA (1:2.5 ratio) targeting AAVS1 in human CD4+ T cells.

The guides were designed to target an AAVS1 locus within the PPP1R12C (protein phosphatase 1 regulatory subunit 12C) gene in human chromosome 19. On-target cutting efficiency of each guide was determined by colony sequencing. The table below shows the number of clones with indels and the total number of analyzed clones, as well as the percentage of NHEJ for each guide assayed in colony sequencing. The various guides of a CRISPR-Cas9/gRNA can target the human AAVS1 locus with a high cutting efficiency. Targeting of the human AAVS1 site resulted in high on-target cutting efficiency and homology-directed Repair (HDR) in the presence of AAV donor template.

| Cas9/gRNA RNP | | |
|---|---|---|
| (Cas9:gRNA ratio) | Indel clones/total clones | % NHEJ |
| P1 (1:1) | 71/73 | 97.3 |
| P1 (1:2.5) | 81/83 | 97.6 |
| P3 (1:2.5) | 45/48 | 93.8 |
| P4 (1:2.5) | 64/66 | 97.0 |
| N1 (1:2.5) | 62/68 | 91.2 |
| N2 (1:2.5) | 61/61 | 100 |
| N3 (1:2.5) | 42/44 | 95.5 |

On-Target Cutting Efficiency of RNPs TargEting Murine FOXP3 in Mouse CD4+ T Cells

Murine CD4+ T cells were isolated from spleens and lymph nodes of C57BL/6 male mice. Isolated cells were then activated using CD3/CD28 Dynabeads followed by Cas9/gRNA RNP electroporation. The molar ratio of Cas9 and guide RNA was 1:2.5. Immediately after electroporation, cells were plated in the wells containing culture media followed by AAV transduction. The murine mT20, mT22, or mT23 spacer sequences targeting murine FOXP3 exon 4 were each used to form gRNA RNP complexes with Cas9 protein. AAVS donor templates containing MND-GFP and homology arm sequences were used for transduction.

Mouse FOXP3 guide RNP on-target cutting efficiency was determined by colony sequencing or ICE analysis in murine CD4 T cells electroporated with ribonuclear protein (RNP) complexed containing mT20, mT22 or mT23. PCR reactions were performed with genomic DNA extracted from each sample to amplify FOXP3 sequences around the expected cut site. Insertion and deletion (INDEL) frequency relative to mock editing was determined using colony sequencing or ICE analysis (Inference of CRISPR Edits). The average of % INDEL was determined from three independent editing experiments. The mean cutting efficiency for RNPs comprising mT20 (92.2%), mT22 (95.3%) or mT23 (93.3%) was greater than 90%.

Murine CD4 T cells were electroporated with FOXP3-specific TALEN targeting a murine FOXP3 exon 4 or Cas9/gRNA RNP as described above, followed by AAV transduction. The AAV donor template contains the MND-GFP and homology arm sequences to upstream and downstream of the nuclease cut site. Homology-directed repair (HDR) using each of the three RNPs resulted in MND-driven GFP expression as measured by flow cytometry. FACS analysis was performed to detect GFP expression as a result of successful editing. As shown in the table below, treatment of RNP targeting murine FOXP3 using mT20 or mT23 and AAV resulted in a higher editing efficiency than treatment of TALEN mRNA and AAV. Blue fluorescent protein (BFP) was used as negative control as compared to green fluorescent protein (GFP) signal.

| Construct | % BFP+ | % GFP+ |
|---|---|---|
| Mock | 0.026 | 0 |
| Talen + AAV | 0 | 14.3 |
| Cas9/mT20 + AAV MND- | 0 | 20.0 |
| GFPki | | |
| Cas9/mT22 + AAV MND- | 0 | 14.7 |
| GFPki | | |
| Cas9/mT23 + AAV MND- | 0 | 23.1 |
| GFPki | | |

Other Embodiments of Murine FOXP3-Directed AAV Donor Templates

A series of murine FOXP3-specific AAV donor templates were prepared containing alternative promoter elements including MND, 0.7UCOE.MND, or PGK promoter followed by GFP coding sequences in-frame with endogenous murine FOXP3 sequences (FIG. 1). AAV donor templates were delivered into murine CD4⁺T cells after Cas9/gRNA-mT23 RNP (Cas9:gRNA in 1:2.5 ratio) electroporation. GFP and FOXP3 levels were determined by flow cytometry at day 2 post editing. nT_{reg} isolated from mouse splenocytes were used to compare FOXP3 expression levels in edT_{reg} vs endogenous FOXP3 levels in natural T_{reg}.

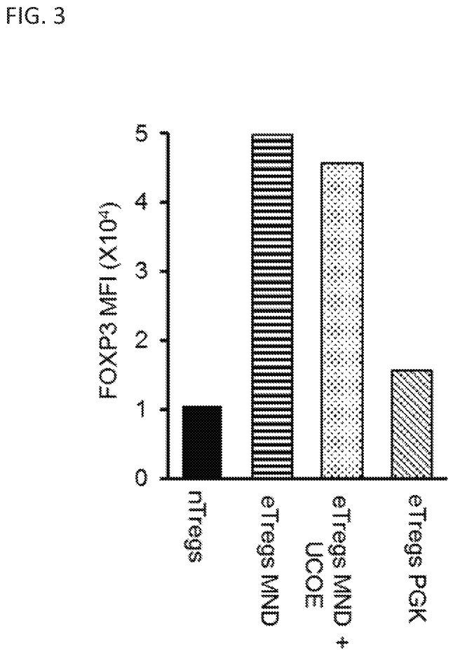

Murine FOXP3 expression was effected with use of the above promoter constructs, but the expression levels varied (FIG. 3).

| | Live CD45+CD4+ gated |
|---|---|
| Experiment | % cells FOXP3+ GFP+ |
| B/6 splenic cells | 0 |
| Mock | 0 |
| AAV #1331 MND promoter | 8.7 |
| AAV #3213 MND with UCOE | 5.0 |
| AAV #3209 PGK | 7.4 |

| Cell Type | FOXP3 MFI (×10⁴) |
|---|---|
| nT_{reg} | 1.0483 |
| eT_{reg} MND | 4.9808 |
| eT_{reg} MND + UCOE | 4.5654 |
| eT_{reg} PGK | 1.5653 |

A series of murine FOXP3-specific AAV donor templates were prepared containing alternative promoter elements including MND, sEFla, or PGK promoter followed by LNFGR and P2A coding sequences in-frame with endogenous murine FOXP3 sequences (FIG. 5H). AAV donor templates were delivered into murine CD4⁺T cells after Cas9/gRNA-mT23 RNP (Cas9:gRNA in 1:2.5 ratio) electroporation. LNGFR and FOXP3 levels were determined by flow cytometry at day 2 post editing.

These data demonstrate that a number of promoters were successfully introduced into an endogenous FOXP3 locus, leading to varying overall levels of FOXP3 in edT_{reg} products.

On-Target Cutting Efficiency of RNPs Targeting FOXP3 in Non-Human Primate CD4+ T Cells

CD4+ T cells from rhesus monkey were isolated from peripheral blood or apheresis products using non-human primate CD4+ T Cell Isolation Kit (Miltenyi). T cell activation was performed by incubating cells with in-house conjugated CD3/CD28 beads for 60 h before electroporation and/or AAV transduction. To test electroporation parameters, BFP mRNA was electroporated and expression of BFP was determined at day 2 post electroporation. To determine AAV serotypes, the constructs containing MND-GFP expression cassette were packaged into various AAV serotypes and then transduced activated CD4+ T cells. GFP expression was analyzed by FACS to determine the transduction efficiency.

The RNPs targeting FOXP3 were tested for their efficiency in editing non-human primate CD4+ T cells. CD4+ T cells were obtained from non-human primate rhesus monkeys. The Cas9/gRNA RNPs comprised T3 (SEQ ID NO: 3), T9 (SEQ ID NO: 5), or R1 (SEQ ID NO: 7) spacer sequence. The Cas9/sgRNA RNP complexes targeted exon 3 in a rhesus FOXP3 locus. Accordingly, each of the RNPs demonstrated high on-target cutting efficiency in rhesus monkey CD4+ T cells, showing from about 70% to about 90% NHEJ by TIDE (Tracking Indels by Decomposition), ICE (Interference of CRISPR Edit), or colony sequencing. This suggested that the human FOXP3-targeting guides could be used in non-human primates due to the species FOXP3 homology.

| | rhFOXP3 cutting efficiency % NHEJ | | |
|---|---|---|---|
| Cas9/gRNA RNP | TIDE | ICE | Colony sequencing |
| T3 | 90.6 ± 0.3 | 89 ± 0.0 | 94.5 ± 0.0 |
| T9 | 69.95 ± 1.55 | 77 ± 3.0 | 89.0 ± 0.0 |
| R1 | 70.75 ± 0.35 | 69 ± 1.0 | 89.47 ± 0.0 |

### Example 3: Expression of a Codon-Optimized cDNA Encoding a FOXP3

TALEN-Mediated Editing to Incorporate FOXP3 Expression

In order to demonstrate that FOXP3 activity can be provided, CD4+ cells were obtained from healthy human subjects and were transfected with (i) a nucleic acid encoding a TALEN, (ii) a donor template encoding a FOXP3 and an AAV vector for expression of a nucleic acid encoding AAV-MND-LNGFR-2A KI (control), or (iii) AAV-MND-FOXP3cDNA-2LNGFR (ID: B in Example 1). Cells expressing the human codon-optimized FOXP3 cDNA showed expression of both FOXP3 and LNGFR as shown in the below table.

| Experiment | % total cells LNGFR+ FOXP3+ |
|---|---|
| TALEN only | 0.01 |
| MND-LNGFR-2A KI and | 28.5 |
| FOXP3 donor template | |
| MND-LNGFR-2A-FOXP3 cDNA | 6.98 |

Comparison Between TALEN-Mediated and Cas9/sgRNA RNP-Mediated Editing

CD4+ cells were obtained from healthy human subjects and were transfected with a nucleic acid encoding a TALEN mRNA, Cas9/gRNA (T3) RNP or Cas9/gRNA (T9) RNP. Cells were then transfected with a viral vector expressing either MND-GFP-KI (described in PCT/US2016/059729, herein expressly incorporated by reference in its entirety) or MND-GFP-FOXP3cDNA (shown in Table 2).

MND-GFP KI was cleavable by the Cas9/gRNA comprising T3 RNP and the Cas9/gRNA comprising T9 RNP, and therefore were not tested in the editing.

The results show that a similar HDR rate was achieved between TALEN and Cas9 mediated editing. However, the data suggested that the homology arm sequences were distant from both TALEN and Cas9 cleavage sites, thus leading to reduced HDR efficiency compared with the positive control. Accordingly, we proceeded to generate modified homology arms. This demonstrated that FOXP3 activity can successfully be provided.

| Experiment | % total cells FOXP3+ GFP+ |
|---|---|
| TALEN mRNA only | 0.13 |
| TALEN mRNA + | 38.1 |
| MND-GFP-KI (positive control) | |
| TALEN mRNA + MND-GFP-FOXP3 | 6.71 |
| cDNA | |
| Cas9/gRNA (T3) RNP + | 9.38 |
| MND-GFP-FOXP3 cDNA | |
| Cas9/gRNA (T9) RNP + | 8.46 |
| MND-GFP-FOXP3 cDNA | |

### Example 4: Modification of Homology Arms of Cas9/sgRNA RNPs

Comparing Editing Rate Between RNPs Comprising T3 and T9 sgRNA Using AAV Donor Templates with Modified Homology Arms Specific for the Respective Guide

CD4+ cells were obtained from healthy human subjects and were transfected with a nucleic acid encoding either a Cas9/sgRNA-T3 RNP or a Cas9/sgRNA-T9 RNP. The AAV donor templates #3063 and #3066 tested included construct A of Example 1, which were FOXP3cDNA-LNGFR derivatives having a 5′- to 3′-coding sequence of:

ITR-HA-MND promoter-FOXP3cDNA-2A-LNGFR-SV40polyA-HA-ITR.

| AAV | | |
|---|---|---|
| donor | Homology | |
| template | arm (HA) | Description |
| #3063 | 0.6 kb | AAV_FOXP3.06_MND.forP3geneartCDS.P2A.LNGFR.pA_06 |
| | homology | for T3 |
| | arm | |
| | sequence | |
| | for T3 | |
| #3066 | 0.6 kb | AAV_FOXP3.06_MND.forP3geneartCDS.P2A.LNGFR.pA_06 |
| | homology | for T9 |
| | arm | |
| | sequence | |
| | for T9 | |

Because AAV donor templates #3063 and #3066 were tailored to be paired specifically with Cas9/gRNA-T3 and Cas9/gRNA-T9, respectively, the editing efficiency was compared between Cas9/gRNA-T3+#3063 and Cas9/gRNA-T9+#3066.

As shown below, the DNA cleavage directed by RNPs comprising T3 and T9 gRNA and 0.6 kb homology arm sequences showed similar HDR efficiency.

| Treatment | % LNGFR+ cells |
|---|---|
| Mock | 0.04 |
| AAV only | 1 |
| Cas9/gRNA-T3 + AAV #3063 | 23 |
| Cas9/gRNA-T9 + AAV #3066 | 27 |

| | Experiment #1 | |
|---|---|---|
| | | Cas9/gRNA (T9) + |
| Homology arm | FOXP3 cDNA-LNGFR | FOXP3 cDNA-LNGFR |
| length (kb) | AAV only | AAV |
| 0.3 | 1.5 | 30.0 |
| 0.45 | 2.2 | 44.7 |
| 0.6 | 2.1 | 43.3 |

| | Experiment #2 | |
|---|---|---|
| | | Cas9/gRNA (T9) + |
| Homology arm | FOXP3 cDNA-LNGFR | FOXP3 cDNA-LNGFR |
| length (kb) | AAV only | AAV |
| 0.3 | 2.5 | 22 |
| 0.45 | 6.5 | 44 |
| 0.6 | 5.4 | 44 |
| 0.8 | 4.3 | 45 |

### Example 5: Phenotyping of Engineered T Cells

Treg_{reg}-Associated Markers

Levels of T_{reg}-associated markers in mock and edited T cell 3 days post editing were determined. The CD4+ cells were obtained from healthy human subjects and were either (i) subjected to mock editing or (ii) subjected to Cas9/sgRNA-T9 RNP and transfected with the AAV donor template FOXP3 cDNA-LNGFR construct with 0.6 kb homology arms as shown in the figures (construct A in Example 1, FOXP3cDNA-LNGFR).

As shown in the table below, the mock control cell did not express the low affinity nerve growth factor receptor (LNGFR) at significant levels. In contrast, LNGFR was expressed with FOXP3 as well as other T_{reg}-associated markers, including ICOS, C1)25, CD45RO, LAG3, and CTLA-4, upon editing with Cas9/sgRNA-T9 RNP+AAV donor template Construct A having 0.6 kb homology arms.

% LNGFR+ Marker+ cells

| | | Cas9/sgRNA-T9 RNP + |
|---|---|---|
| | | AAV construct A (0.6 kb |
| Marker | mock | homology arms) |
| FOXP3 | 0.22 | 40.1 |
| ICOS | 0.086 | 46.8 |
| CD25 | 0.074 | 40.3 |
| CD45RO | 0.15 | 41.6 |
| CD127 | 0.074 | 3.7 |
| LAG3 | 0.11 | 10.9 |
| CTLA-4 | 0.15 | 31.4 |
| Helios | 0.095 | 2.9 |

Cytokine Production Upon PMA/Inomycin Stimulation

Edited T cells were then phenotyped. Cells carrying Construct A were able to produce cytokines upon PMA/Inomycin stimulation.

### Example 6. Evaluation of AAV Donor Templates with Various Expression Cassettes

Experiments were performed to test AAV donor templates with various expression cassettes. P2A (porcine teschovirus-1 2A) or IRES (internal ribosome entry site) were compared for multi-cistronic expression using vectors comprising FOXP3 cDNA-P2A-GFP vs. FOXP3 cDNA-IRES-GFP. Also compared were the relative orientations of FOXP3 cDNA and selection marker (FOXP3-P2A-LNGFR vs LNGFR-P2A-FOXP3) as well as the FOXP3 staining reagents and protocols to finalize the methods.

The following constructs (in the 5′- to 3′-direction) were evaluated. HA indicated homology arms.

### (0.25 kb HA)-MND-FOXP3cDNA-2A-GFP-WPRE-pA-(0.25 kb HA)

### (0.25 kb HA)-MND-FOXP3cDNA-IRES-GFP-WPRE-pA-(0.25 kb HA)

(0.45 kb HA)-MND-LNGFR-2A-FOXP3 cDNA-WPRE-pA-(0.6 kb HA)
(0.45 kb HA)-MND-FOXP3 cDNA-2A-LNGFR-WPRE-pA-(0.6 kb HA).

T cells were collected from the PBMC of healthy human donors and were edited with Cas9/sgRNA-T9 (1:2.5 Cas9:gRNA) RNP and AAV donor templates: FOXP3 cDNA-IRES-EGFP, FOXP3 cDNA-P2A-EGFP, LNGFR-P2A-FOXP3 cDNA, or FOXP3 cDNA-P2A-LNGFR. The cells were stimulated with Phorbol 12-myristate 13-acetate (PMA), Inomycin and GolgiStop for five hours. Cell fixation and permeabilization was performed overnight using True-Nuclear Transcription Factor Buffer Set (Biolegend, San Diego, Calif. USA). FACs analysis was performed to analyze eGFP expression (FOXP3 cDNA-IRES-EGFP, FOXP3 cDNA-P2A-EGFP) and LNGFR+ expression in the cells (LNGFR-P2A-FOXP3 cDNA and FOXP3 cDNA-P2A-LNGFR). The cells were also analyzed for CD127+, CD25+, and FOXP3 expression at 7 and 15 days. The tables below summarize the results of these studies.

| | % GFP+ Marker+ cells | |
|---|---|---|
| Marker | FOXP3 cDNA-IRES-EGFP | FOXP3 CDNA-P2A-EGFP |
| FOXP3 | 7.0 | 9.1 |
| CD25 | 23.7 | 43.2 |
| CD127 | 0.009 | 0.1 |

| | % LNGFR+ Marker+ cells | |
|---|---|---|
| Marker | LNGFR-P2A-FOXP3 cDNA | FOXP3 CDNA-P2A-LNGFR |
| FOXP3 | 8.5 | 9.5 |
| CD25 | 32.9 | 40.5 |
| CD127 | 0.01 | 0.01 |

True Nuclear 1 Hour Fixation/Permeabilization

| | % GFP+ Marker+ cells | | % LNGFR+ Marker+ cells | |
|---|---|---|---|---|
| | FOXP3 cDNA- | FOXP3 cDNA- | LNGFR-P2A- | FOXP3 cDNA- |
| Marker | IRES-EGFP | P2A-EGFP | FOXP3 cDNA | P2A-LNGFR |

FOXP3
CD25
CD127

True Nuclear Overnight Fixation/Permeabilization:

| | % LNGFR+ Marker+ cells |
|---|---|
| Marker | FOXP3 cDNA-P2A-LNGFR |
| FOXP3 | 10 |
| CD25 | 25 |
| CD127 | 5 |

eBioscience 1 Hour Fixation/Permeabilization:

% LNGFR+ Marker+ cells

| | LNGFR-P2A- | FOXP3 cDNA- |
|---|---|---|
| Marker | FOXP3 cDNA | P2A-LNGFR |
| FOXP3 | 15.4 | 12.7 |
| CD25 | 17.3 | 26 |
| CD127 | 4.6 | 1.0 |

At day 7 and 14, post enrichment, the cells were further analyzed for viability and analysis of GFP expression (FOXP3 cDNA-IRES-EGFP, FOXP3 cDNA-P2A-EGFP) as summarized in the tables below.

| | FOXP3 | FOXP3 | FOXP3 | FOXP3 |
|---|---|---|---|---|
| | cDNA-IRES- | cDNA-IRES- | cDNA-P2A- | cDNA-P2A- |
| % cells | (EGFP−) | (EGFP+) | (EGFP−) | (EGFP+) |
| lympho- | 22.9 | 30.1 | 33.3 | 33.9 |
| cytes | | | | |
| SSC-A, | 89.5 | 83.1 | 87.6 | 83.6 |
| FSC-W | | | | |
| subset | | | | |
| GFP+ | 0.34 | 16.6 | 0.27 | 21.0 |

| | LNGFR-P2A- | FOXP3 cDNA- |
|---|---|---|
| % cells | FOXP3 cDNA | P2A-LNGFR |
| lymphocytes | 14.6 | 9.2 |
| SSC-A, FSC-W | 93.0 | 92.3 |
| subset | | |
| GFP+ | 10.8 | 31.3 |

As shown from the above results, the construct comprising P2A performed better than IRES, because the AAV donor template FOXP3 cDNA-P2A-LNGFR resulted in a higher MFI of LNGFR than FOXP3 cDNA-IRES-LNGFR when used for transfection in conjunction with Cas9/sgRNA-T9 RNP in editing CD4+ T cells from healthy human donors.

As for LNGFR/FOXP3 staining, the eBioscience buffer set afforded better fixation/permeabilization results than True Nuclear buffer set.

There is a difference between beads/column-based and cell sorting-based enrichment. Beads/column can be used to select all positive population (mid and high). A sorter can also specifically select population with high level, which can contribute to difference in expansion, purity, and phenotypes, etc. This can then also be used to compare LNGFR+ sorted vs beads-enriched in the next experiment.

PMA stimulation for cytokine analysis was also shown to induce endocytosis of CD4. The next step was to test different stimulation protocols and cytokine staining for LNGFR+ cells.

### Example 7: Gene Editing to Integrate MND-GFP-Murine FOXP3 cDNA at Murine FOXP3 Locus

Gene editing was performed with TALEN to integrate MND GFP-murineFOXP3cDNA at a murineFOXP3 Locus. The next step was to perform phenotyping 2 days post cell sorting. For the experiments, mock cells, and cells expressing MND-GFPki and MND-GFPmFOXP3CDS were used for the phenotyping analysis. Gene-editing mediated integration of MND-GFP-murineFOXP3cDNA at murine FOXP3 Locus resulted in expression of murine FOXP3cDNA and T_{reg}-like phenotype, high CD25, and high CLTA-4.

| | % Marker+ FOXP3+ Cells | | |
|---|---|---|---|
| | % | % CD25+/ | %CTLA-4+/ |
| Cell Type | GFP+FOXP3+ | FOXP3+ | FOXP3+ |
| Mock-edited | 0.35 | 1.13 | 1.53 |
| MND-GFPki edited | 66.3 | 66.7 | 57.6 |
| MNDGFPmFOXP3CDS | 46.5 | 49.0 | 43.5 |

### Example 8: Gene Editing of Non-Human Primate Cells

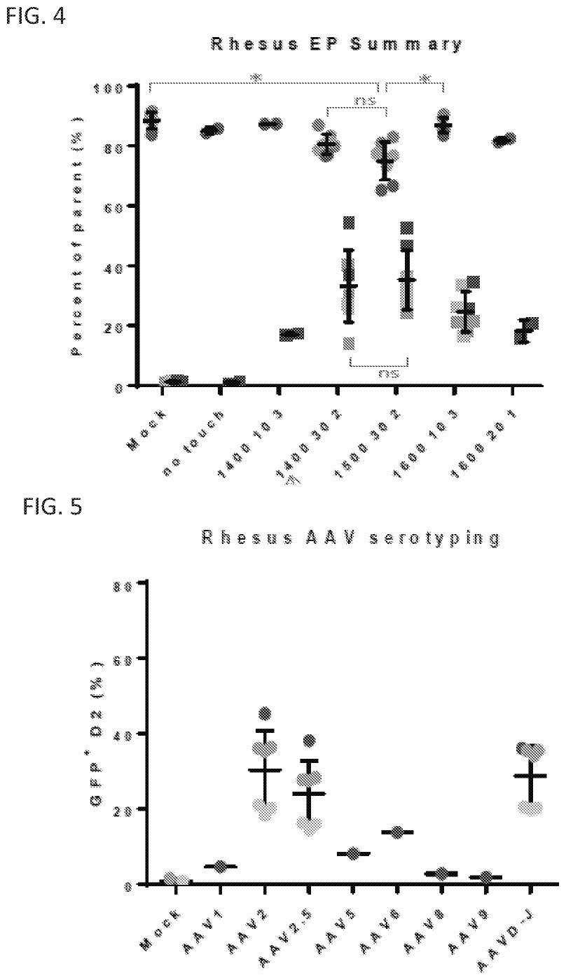

Gene editing was performed on non-human primate cells using Rhesus CD4+ cell electroporation. Shown in FIG. 4 is a Rhesus monkey electroporation summary for CD4+ cells from three rhesus monkeys, showing the viability of cells after electroporation and their ability to express BFP (blue fluorescent protein). BFP mRNA was used to test electroporation conditions. % BFP+indicated the electroporation efficiency. The electroporation condition−1400 V, 20 ms pulse, 2 pulses total−afforded about 20-50% BFP+ cells without significant loss of cell viability compared with control.

The table below shows data of efficiency of transduction using different AAV subtypes in the T cells derived from non-human primate rhesus monkey. A MND-GFP construct was packaged into different AAV serotypes (AAV-2, AAV-2.5 and AAV-DJ) and used to transduce non-human primate cells isolated from rhesus monkeys #1 and #2. Flow plots show GFP expression observed at day 2 post transduction.

Mock Editing

| | % lymphocytes (as | |
|---|---|---|
| | determined from SSC-A | % GFP+ (as determined |
| Donor | vs. FSC-A) | from SSC-A vs. GFP) |
| #1 | 88.7 | 0.71 |
| #2 | 92.6 | 1.41 |

Editing with Cas9/sgRNA-T9 RNP and AAV as Indicated

| | % lymphocytes (as | |
|---|---|---|
| | determined from SSC-A | % GFP+ (as determined |
| Donor/AAV subtype | vs. FSC-A) | from SSC-A vs. GFP) |
| #1/AAV-DJ | 88.4 | 20.2 |
| #2/AAV-2 | 92.8 | 35.4 |
| #2/AAV-2.5 | 87.2 | 27.6 |

### Example 9: Expression of mRNA Encoding a FOXP3 from Non-FOXP3 Genetic Locus

AAV Donor Template Design for TCRa

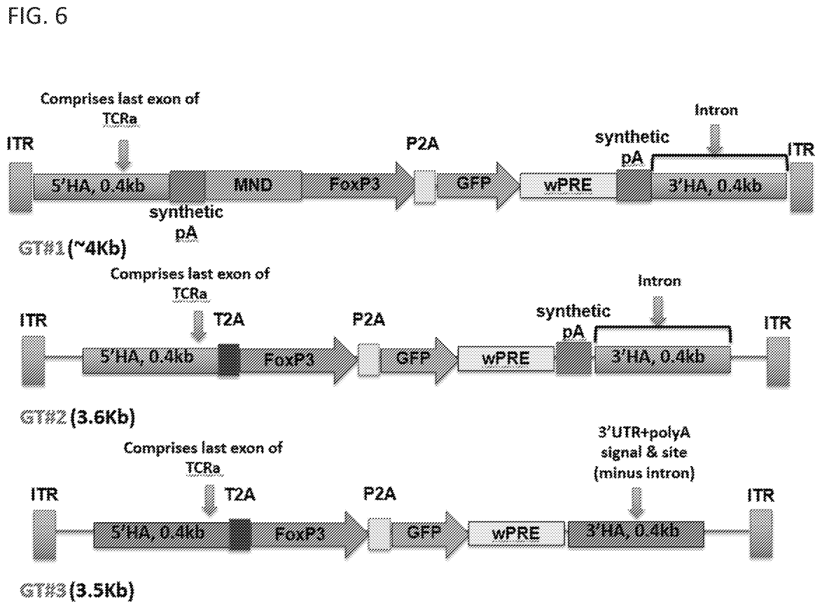

FIG. 6 shows the design of the TCRa gene trap constructs used. The TCRa spacer sequences (“Guide #1” through “Guide #4”, SEQ ID NOs: 125-128, respectively) targeted the last exon (exon 6) of TCRa and were checked using COSMID.

| Guide | Sequence | SEQ ID NO | PAM sequence |
|---|---|---|---|
| #1 | ATGCAAGCCCATAACCGCTG | 125 | TGG |
| #2 | CAAGAGGCCACAGCGGTTAT | 126 | GGG |
| #3 | CCAAGAGGCCACAGCGGTTA | 127 | TGG |
| #4 | TTCGGAACCCAATCACTGAC | 128 | AGG |

Within a Cas9/gRNA RNP, Guide #1 (SEQ ID NO:125) utilized the MND promoter to drive the expression of FOXP3 cDNA and the selection marker GFP. Guide #2 (SEQ ID NO: 126) and Guide #3 (SEQ ID NO: 127) each used the endogenous TCRa (TRAC) promoter to express FOXP3 cDNA and the GFP marker. These three constructs were designed for mRNA expression of FOXP3 from a non-FOXP3 genetic locus, specifically, TCRa. The constructs were TCRa gene trap constructs:
1) 5′HA (0.4 kb)-pA-P2A-MND-FOXP3-GFP-wPRE-synthetic PA-3′HA (0.4 kb) (construct is 4 kb),
2) 5′HA (0.4 kb)-T2A-FOXP3-P2A-GFP-wPRE-syntheticPA-3′HA (0.4 kb) (construct is 3.6 kb) and
3) 5′HA (0.4 kb)-T2A-FOXP3-P2A-GFP-wPRE-3′HA (0.4 kb) (without intron) (construct is 3.5 kb).

Cell Editing with TCRa Site Targeting

The TCRa targeting samples that used a 63 h T cell bead stimulation layout (NHEJ/HR). The samples were tested for editing efficiency from cells that are stimulated with CD3/CD28 Dynabeads for 63 h prior to editing.

Edited cells were analyzed at day 7 post editing from CD4+ cells from healthy human donors that were activated for 63 h prior to editing. The results of the genome editing using Cas9/gRNA (1:1) and indicated AAV donor template are summarized in the table below. In each case, expression of the GFP marker was effectively introduced.

| Guide sequence in gRNA | % GFP+ cells after transduction |
|---|---|
| Control | <0.1 |
| Guide #1 | 17 |
| Guide #2 | 16.5 |
| Guide #3 | 4.7 |

AAV Donor Templates for AAVS1 Site Editing

AAV donor templates for AAVS1 site editing were used. The following general structures of the donor templates included the following (HA=homology arm):

to determine bi-allelic editing efficiency:

### ITR-HA-MND-GFP-WPRE3-pA-HA-ITR,

### ITR-HA-MND-BFP-WPRE3-pA-HA-ITR,

to edit with FOXP3cDNA AAV template:
ITR-HA-MND-FOXP3 cDNA-2A-LNGFR-pA-HA-ITR,
and to use bi-allelic editing in order to express FOXP3cDNA and DISC:

### ITR-HA-MND-CISCβ-2A-FRB-2A-marker-pA-HA-ITR, and

### ITR-HA-MND-CISCγ-2A-FOXP3cDNA-2A-LNGFR-pA-HA-ITR

The AAVS1 site editing efficiency using Cas9/gRNA RNP with P1 and N2 guides with the AAV donor template—ITR-HA-MND-GFP-WPRE3-pA-HA-ITR—showed that the % GFP^{high} population after Day 8 post-editing with RNP and AAV donor template treatment ranged from 58-72%.

### Example 10: Exemplary T Cell Gene Editing Protocol with Cas9/gRNA RNP and an AAV Donor Template

Frozen human PBMCs were rapidly thawed and washed, and CD4+ T cells were collected using a negative selection kit (STEMCELLTech EasySep CD4+ Enrichment Kit). CD4+ cells (supernatant after negative selection on beads) were resuspended in T Cell Culture Media (RPMI 1640 with 20% FBS, 1×Glutamax (2 mM L-alanyl-L-glutamine dipeptide), 55 μM 2-mercaptoethanol and 50 ng/mL human IL-2) at 0.5 million cells/mL, and activated with T Expander CD3/CD28 Dynabeads at a 3:1 bead-to-cell ratio. The cells were cultured 3 days in 5% CO₂ at 37° C. 72 hours after CD3/CD28 bead addition, beads were removed and cells were cultured overnight as above.

After washing, cells were resuspended in electroporation Buffer P3 (Lonza), Buffer T (Neon), or Maxcyte electroporation buffer according to the manufacturer's recommendations, and the appropriate RNP mix was added (SpyFI Cas9 (Aldevron, Fargo, N. Dak. USA) mixed with CAS9 RNP/T9 at 1:2.5 molar ratio in the appropriate electroporation buffer). Electroporation or nucleofection was performed using Lonza 4D with program code DN-102 or EO-115, Neon with 1420V/10 ms/3pulse, or Maxycte with the expanded T cell 1-OC program. Cells were then collected in pre-warmed T Cell Culture Media along with the addition of 20% (v/v) AAV6 donor template and incubated at 37° C. for 24 h before adding 1 volume of media to dilute the AAV. HDR efficiency was analyzed approximately 48 h after editing by flow cytometry. LNGFR microbeads-mediated magnetic column selection was performed approximately 72 h post editing. Enriched cells were then transferred to appropriately sized G-Rex® flasks (according to manufacturer's protocol, WilsonWolf, St. Paul, Minn.) and cultured for an additional 7 days with the T cell Culture Media containing 100 ng/mL IL2. In addition, cells were treated with 100 nM rapamycin at time of seeding into G-Rex® flasks and half volume of culture media was changed every 2-3 days during the 7-day expansion in G-Rex® flasks. Cells were analyzed, then viably frozen or used immediately.

### Example 11. Characteristics of Cells Edited Using Exemplary Gene Editing Protocol

Evaluating Editing Rate Using the Exemplary Protocol

The efficiency of editing using the exemplary protocol described in Example 10 with the AAV donor template that had 0.6 kb arms of FOXP3 homology at both the 5′ and 3′ ends was evaluated in 13 different experiments, using T cells from 6 different donors. The average HDR rate, as assessed by flow cytometry on day 2, was at or about 34% (see table below).

| Conditions | % LNGFR+ cells |
|---|---|
| Mock | <1 |
| AAV only | 2 |
| AAV + SpyFi Cas9/gRNA-T9 (1:2.5) RNP | 34 |

Cell Surface Expression of Canonical Thymic T_{reg} Markers in Edited Cells

Immunophenotyping was performed on cells edited using the exemplary editing protocol described in Example 10 at 3 days post-editing using flow cytometry. Staining of CD4, LNGFR, CD25, CD127, LAG3, CTLA-4, and CD45R0 was performed following a standard surface staining procedure. Subsequently, cells were fixed and permeabilized using the True-Nuclear Transcription Factor kit (Biolegend) before staining with antibodies against FOXP3, and Helios. LNGFR⁺ cells (signifying successfully edited cells) were phenotypically similar to naturally occurring thymic T_{reg} (tT_{reg}), with high FOXP3, CD25, CTLA4, ICOS, and LAG3, and low CD127 levels. CD45RO staining showed that the edited cells were consistent with a memory phenotype. Helios levels were not up-regulated in the edited cells.

| Marker | % LNGFR+ Marker+ cells by FACS |
|---|---|
| FOXP3 | 40.1 |
| ICOS | 46.8 |
| CD25 | 40.3 |
| CD45RO | 41.6 |
| CD127 | 3.66 |
| LAG3 | 10.9 |
| CTLA-4 | 31.4 |
| Helios | 2.86 |

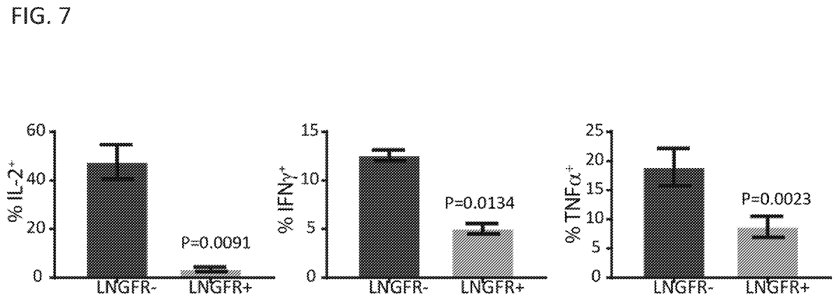

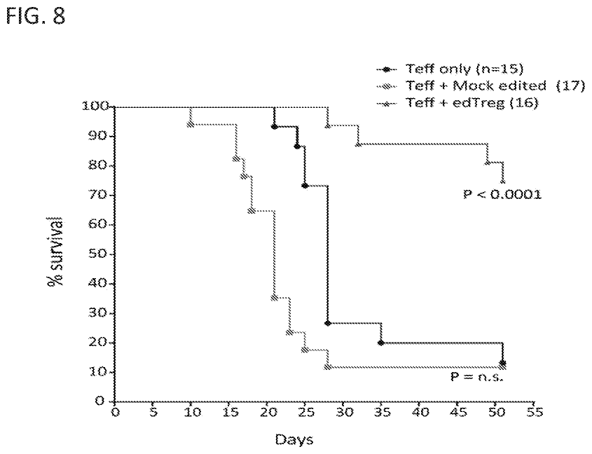

An intracellular cytokine labeling assay was also performed, wherein cells were activated with PMA/Ionomycin to mimic an antigen signal, then fixed and permeabilized to detect cytokines. Inflammatory cytokines that would normally be highly upregulated in effector T cells were not upregulated in LNGFR⁺ cells, but were upregulated in LNGFR− cells (FIG. 7), consistent with LNGFR+ cells exhibiting a tT_{reg}-like phenotype.

To confirm that the cytokine suppression observed was due to FOXP3, and not other aspects of the editing procedure, a corresponding editing procedure was performed in parallel but using an AAV donor template that had a point mutation in the coding sequence for FOXP3. This mutation, which was found in an IPEX subject, resulted in an R397W amino acid substitution that rendered FOXP3 non-functional. The FOXP3 R397W mutant protein was expressed at a comparable level to wild-type FOXP3 under the gene editing conditions of the exemplary protocol of Example 10. For instance, the % LNGFR+FOXP3+ cells were comparable by FACS (49.2% wild-type; 64.9% R397W mutant).

However, there was no suppression of the inflammatory cytokines tested (IL-2 and TNFα, see table below) in the edited T cells expressing FOXP3 R397W mutant, in contrast to the behavior of the edited T cells expressing wild-type FOXP3.

| Cell Cytokine Characteristics | | WT FOXP3 | R397W FOXP3 |
|---|---|---|---|
| % IL-2+ | LNGFR− | 70 | 71 |
| | LNGFR+ | 21 | 73 |
| % TNFα+ | LNGFR− | 43 | 44 |
| | LNGFR+ | 36 | 47 |

### Example 12: LNGFR Enrichment and Expansion of LNGFR-Enriched Cells in Culture

For certain applications (e.g., clinical applications), the ability to select edited cells using a cell surface tag could be useful to reduce the fraction of non-edited cells (that have a proliferation advantage in culture). To test this, on Day 3 after editing using the exemplary editing protocol described in Example 10, (CD271) LNGFR microbeads (Miltenyi) or MACSelect LNGFR microbeads (Miltenyi) were used to enrich successfully edited cells following manufacturer's suggested protocol.

Flow cytometry was used to monitor LNGFR+ cell enrichment before and after enrichment, as well as after further expansion in G-Rex® flasks (see table below). At the end of the 7-day expansion, LNGFR⁺ cells had expanded an average of at or about 42-fold, and LNGFR⁺ cells represented at or about 91% of the final cell preparation.

| Conditions | % LNGFR+ cells |
|---|---|
| Day 2: mock edited | 7.7 |
| Day 2: edited | 32 |
| Day 3: LNGFR enriched | 99 |
| Day 10: LNGFR enriched cells culture 1 week | 98 |

### Example 13: Testing Immunosuppression in a CD4⁺T Cell Adoptive Transfer Inflammatory (CATI) Mouse Model

NOD-scid-IL2Rg^{Null} (NSG) mice are immunologically incompetent and can be engrafted with human T cells. When delivered after a dose of total body irradiation, human CD4 T cells have been reported to promote an inflammatory response dependent on murine MHC-II (Covassin, L. et al. (2011). Clin. Exp. Immunol. 166(2):269-280). Inflammatory responses included the activation and expansion of the human CD4 T cell population, up-regulation and release of pro-inflammatory human cytokines such as IL-2 and IFN-γ, and resulted in damage to tissues where the cells localized, including the gut, lung and skin. It has been shown that autologous thymic regulatory T cells (tT_{reg}) can suppress the activation of the CD4 T_{eff} cells in this model, providing a model system for testing the immunosuppressive properties of the edited regulatory T cells described herein.

The CD4 adoptive transfer inflammation (CATI) model was used to evaluate the edT_{regs}. Mice were irradiated with 200 rads each. NSG mice were engrafted with 4×10⁶ autologous CD4+T effector (T_{eff}) cells containing: i) T_{eff} only (n=15), ii) T_{eff}+mock-edited cells (n=17), or iii) T_{eff}+edT_{reg} (n=16) edited using the exemplary editing protocol described in Example 10. After 14 days post-infusion, peripheral blood was collected from a subset of four mice from each of the mock and edT_{reg} cohorts, that were sacrificed and subjected to analysis for the presence of human T cells. Mice were euthanized at humane endpoints, such as >20% loss of body weight. There were increased proportions and numbers of human CD4⁺ CD45RO+ cells in the mock cell-treated group vs. those mice treated with edT_{reg} (65% cells in mock vs. about 20% in edT_{reg}) (p=0.0034). In the edT_{reg} group, about 40% of these CD4+CD45RO+ cells were LNGFR⁺ edT_{reg} as compared to 0.08% of mock edited cells (p=0.0037). Most of the mice within the two negative control groups (T_{eff} only, or T_{eff}+mock-edited cells) were euthanized within the first 3 weeks post-transfer due to pre-determined humane endpoints, generally, excessive weight loss.

The treatment with edT_{reg} significantly delayed onset and severity of inflammatory T cell morbidity in the NSG mice as compared with no or mock treatment (FIG. 8). For instance, 75% of the mice (12/16 of the cohort) engrafted with T_{eff}+edT_{reg} cells survived for 50 days, while only about 10% of the mice in the other cohorts (T_{eff} only or T_{eff}+mock) survived.

### Example 14. Enhancing Efficiency of AAV Donor Templates to Generate edT_{reg} Cell Preparations

### Evaluation of the Effect of WPRE Element on FOXP3, GFP, and LNGFR Expression Levels.

Results

We evaluated the effect of full-length and truncated WPRE on FOXP3 expression using the FOXP3cDNA-P2A-GFP and FOXP3cDNA-P2A-LNGFR donor templates.

First, to determine whether WPRE increases expression levels of FOXP3 cDNA transgene, tests were first performed in cells edited with the FOXP3cDNA-P2A-GFP donor templates. Woodchuck Hepatitis Virus (WHP) Posttranscriptional Regulatory Element (WPRE)-mediated enhancement of protein expression has been shown for many transgenes and was therefore included in AAV donor design to generate edT_{reg}. The AAV donor templates contained either the full length or truncated WPRE (WPRE6, WPRE3, or WPREr3). In these studies, we used FOXP3-specific TALENs to generate DNA double-strand breaks followed by AAV-mediated donor template delivery.

The AAV donor templates used for this evaluation, with various versions of WPRE and the corresponding virus identification number (ID), are shown below. The construct used was:

ITR-(5′-HA)-MND-FOXP3cDNA-2A-GFP-WPRE-pA-(3′-HA)-ITR.

HA as used above indicated the 5′- or 3′-homology arm. With regard to the specific WPRE element used, the following notation is indicated: “WPRE6”=full length WPRE (˜600 bp), “WPRE3”=truncated WPRE (˜300 bp), “WPREr3”=reverse complement of WPRE3 (˜300 bp).

| AAV donor | |
|---|---|
| template | Construct label |
| 3017 | pAAV_FOXP3.025_MND.FOXP3cDNA.P2A.GFP.WPREr3.pA_025 |
| 3018 | pAAV_FOXP3.025_MND.FOXP3cDNA.P2A.GFP.WPRE6.pA_025 |
| 3019 | pAAV_FOXP3.025_MND.FOXP3cDNA.P2A.GFP.WPRE3.pA_025 |

AAV donor template #3018 comprised the MND promoter at the 5′-end of FOXP3cDNA-P2A-GFP cDNA, and the full length sequence of WPRE (WPRE6, ˜600 bp) followed by SV40-polyA signal at the 3′ end of the FOXP3cDNA-P2A-GFP cDNA. AAV donor templates #3017 and #3019 were similar, except that the WPRE6 was replaced by the truncated WPRE sequence (WPRE3, ˜300 bp) in #3017, and the reversed complement of WPRE3 (WPREc3) was used in #3019. All three AAV donor templates were flanked at both 3′- and 5′-ends with homology arms.

All three conditions led to FOXP3+ and/or GFP+ cells. The table below shows editing efficiency at day 4 post editing, as assessed by flow cytometry. FOXP3 expression was detected with all three constructs.

Percentage of FOXP3+ and/or GFP+ cells at Day 4 after AAV treatment.

| AAV donor | % GFP+/ | % GFP−/ | % GFP+/ |
|---|---|---|---|
| template | FOXP3− | FOXP3+ | FOXP3+ |
| #3017 | 0.42 | 7.37 | 1.29 |
| #3018 | 1.00 | 13.1 | 1.70 |
| #3019 | 0.28 | 8.50 | 1.94 |

All cell populations after treatment with the corresponding AAV donor template shown above exhibited levels of T_{reg} associated markers (CD25, CD127, and CTLA-4) consistent with a T_{reg} phenotype for the HDR edited cell population (GFP+FOXP3+). Thus, inclusion of WPRE in the donor template afforded expression of the encoded FOXP3.

Then, to determine the degree to which the WPRE element influenced the expression levels of FOXP3 cDNA transgene, tests were performed in cells edited with the FOXP3cDNA-P2A-LNGFR donor templates, using FOXP3-specific TALENs to generate DNA DSB followed by AAV-mediated donor template delivery.

The AAV donor templates used for this evaluation, with various versions of WPRE and the corresponding virus identification number (ID), are shown in the table below. The construct used was:

ITR-(5′-HA)-MND-FOXP3cDNA-2A-LNGFR-WPRE-pA-(3′-HA)-ITR.

HA as used above indicated the 5′- or 3′-homology arm. With regard to the specific WPRE element used, the following notation is indicated: “WPRE6”=full length WPRE (˜600 bp), “WPRE3”=truncated WPRE (˜300 bp), “WPREr3”=reverse complement of WPRE3 (˜300 bp).
Description of the AAV donor templates comprising WPRE sequences, or no WPRE.

| AAV | Homology arm (kb) 5′_3′ | Description | Label |
|---|---|---|---|
| 3020 | 0.45_0.6 | truncated WPRE, reverse | 045_WPREr3 |
| 3021 | 0.25_0.25 | complement orientation | 025_WPREr3 |
| 3023 | 0.45_0.6 | truncated WPRE | 045_WPREC3 |
| 3024 | 0.45_0.6 | Full length WPRE | 045_WPRE6 |
| 3045 | 0.45_0.6 | No WPRE | 0.45_No WPRE |

Generally, and as shown above, AAV donor templates #3020, 3021, 3023, 3024, and 3045 comprised the MND promoter at the 5′-end of FOXP3cDNA-P2A-LNGFR, and a version of WPRE or WPRE absent, followed by SV40-polyA signal, at the 3′ end of the FOXP3-GFP cDNA. The length of the homology arms on the 5′ and 3′ ends of each AAV donor template are shown in the table.

All of the evaluated AAV donor templates led to comparable levels of FOXP3 expression, as summarized in the table below.

Percentage of cells at Day 4 after
treatment with AAV donor template

| AAV donor | % LNGFR+/ | % LNFGR−/ | % LNGFR+/ |
|---|---|---|---|
| template | FOXP3− | FOXP3+ | FOXP3+ |
| 3020 | 0.49 | 7.15 | 3.35 |
| 3021 | 0.17 | 6.67 | 1.22 |
| 3023 | 1.94 | 6.19 | 5.33 |
| 3024 | 0.15 | 7.65 | 0.83 |
| 3045 | 0.88 | 6.80 | 3.40 |

These results indicated that inclusion of a WPRE element was not required for high-level FOXP3 expression. The AAV donor template #3045, lacking a WPRE, induced the expression of FOXP3 in a total of 10.2% cells (LNGFR−/FOXP3+ and LNGFR+/FOXP3+ cells combined), which is a similar result when compared to the other AAV donor templates that included a WPRE sequence, e.g. AAV donor templates #3020, #3021 and #3023 that induced FOXP3 expression in a total of 10.5%, 7.89%, and 11.52% of the cells, respectively. Accordingly, WPRE elements were not included in the subsequent AAV donor templates used in subsequent figures.

Methods

We tested the ability of Ubiquitous Chromatin Opening Element (UCOE) to stabilize MND-driven FOXP3cDNA expression. This element can function to reduce silencing and limit negative impact of promoter elements. Ubiquitous Chromatin Opening Element (UCOE) is generally used to create a transcriptionally active chromatin structure around integrated transgenes and can function to reduce silencing and limit negative impact of promoter elements.

To determine the stability of FOXP3 expression in edited cells, FOXP3-specific MND.GFP knock-in AAV donor templates with or without UCOE variants were used in human FOXP3 gene editing, in combination with FOXP3-targeting TALENs. A successful editing would therefore lead to GFP in-frame fused with endogenous FOXP3, as the donor templates, as described on FIG. 44A, were designed to create an in-frame fusion of the GFP cassette to part of the Exon of FOXP3 where the TALEN cut site was located. The GFP cassette on the donor templates was located downstream of the MND promoter, itself downstream, or not at all, of the forward or reverse 07UCOE sequence. After gene editing, GFP expression was tracked for 21 days by flow cytometry to determine whether silencing occured in the edited cells in vitro and whether the presence of UCOE variants stabilized the MND-driven GFP.FOXP3 fusion protein expression. We observed that GFP/FOXP3 was stable for a period of 21 days with or without the UCOE, as shown on FIG. 44B, suggesting that UCOE shielded donor works effectively and may be useful in future production of select preparations. These results demonstrated stable expression of GFP/FOXP3 over time in vitro, with or without inclusion of the UCOE element. These findings indicated that UCOE shielded donor worked effectively

### Evaluation of Ubiquitous Chromatin Opening Element (UCOE) in the Stabilization of MND-Driven FOXP3cDNA Expression.

Results

We tested the ability of Ubiquitous Chromatin Opening Element (UCOE) to stabilize MND-driven FOXP3cDNA expression. This element can function to reduce silencing and limit negative impact of promoter elements. Ubiquitous Chromatin Opening Element (UCOE) is generally used to create a transcriptionally active chromatin structure around integrated transgenes and can function to reduce silencing and limit negative impact of promoter elements.

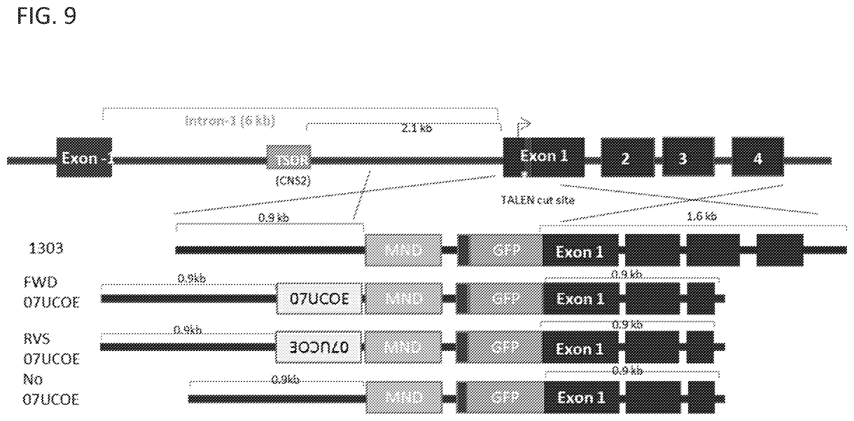

To determine the stability of FOXP3 expression in edited cells, FOXP3-specific MND.GFP knock-in AAV donor templates with or without UCOE variants were used in human FOXP3 gene editing, in combination with FOXP3-targeting TALENs. A successful editing would therefore lead to GFP in-frame fused with endogenous FOXP3, as the donor templates, as described on FIG. 9, were designed to create an in-frame fusion of the GFP cassette to part of the Exon of FOXP3 where the TALEN cut site was located. The GFP cassette on the donor templates was located downstream of the MND promoter, itself downstream, or not at all, of the forward or reverse 07UCOE sequence.

After gene editing, GFP expression was tracked for 21 days by flow cytometry to determine whether silencing occurred in the edited cells in vitro and whether the presence of UCOE variants stabilized the MND-driven GFP.FOXP3 fusion protein expression. We observed that GFP/FOXP3 was stable for a period of 21 days with or without the UCOE, suggesting that the UCOE regulatory element worked effectively and may be useful in future production of select preparations. These results demonstrated stable expression of GFP/FOXP3 over time in vitro, with or without inclusion of the UCOE element. These findings indicated that a UCOE regulatory element may be useful to stabilize expression of a FOXP3.

Methods

CD4+ T cells isolated from adult healthy donors were activated with anti-CD3/CD28 beads for 48 h at cell concentration between 0.5-1 milion/ml. After an overnight rest post beads removal, cells were electroporated with human FOXP3-specific TALEN mRNAs using Neon transfection system. AAV donor templates containing FOXP3cDNA-P2A-GFP and FOXP3cDNA-P2A-LNGFR with or without WPRE varients were then added to cell culture 2 h after transfection followed by a 24-hour incubation time at 30 C. After incubation, fresh media were added into culture to dilute AAV to reduce AAV-related toxcicity. HDR efficiency was analyzed assessed by flow cytometry by % GFP+ or % LNGFR+ at day2 post editing. FACS analysis was performed for LNGFR and FOXP3 expression at day 4 post editing.

Evaluation of FOXP3 and Other T_{reg}-Associated Markers in edT_{reg} Cells Expressing a LNGFR Selectable Marker.

Results

We then studied the introduction of a cis-linked surface marker, LNGFR, for potential use of anti-LNGFR antibodies for purification of edT_{reg} preparations expressing this marker.

In this experiment, we tested different AAV donor templates designed to achieve this goal, AAV #3066, #3098, and #3117, as further described below. The AAV donor templates contained a cis-linked LNGFR marker, either at the 3′-end of FOXP3cDNA (AAV #3066 and 3098), or its 5′-end (AAV #3117). The AAV donor templates were either Construct A or Construct B as described above and summarized below, where HA=homology arm:

(A) ITR-HA-MND-FOXP3 cDNA-2A-LNGFR-pA-HA-ITR,

(B) ITR-HA-LNGFR-2A-FOXP3cDNA-pA-HA-ITR.

| AAV donor | Construct | |
|---|---|---|
| template | type | Description (5′- and 3′-homology arms omitted) |
| #3066 | A | MND-FOXP3cDNA-P2A-LNGFR-pA |
| #3098 | A | MND-FOXP3cDNA.R397W-P2A-LNGFR-pA |
| #3117 | B | MND-LNGFR-P2A-FOXP3cDNA-pA |

These AAV donor templates were cotransfected with mock or RNPs targeting endogenous FOXP3 in CD4+ cells. The cells were collected and analyzed by immunostaining and flow cytometry 6 days after editing.

The percentage of cells expressing LNGFR (LNGFR+) after being transfected with the one of the three constructs (AAV #3066, 3098, or 3117) with or without RNPs directed against endogenous FOXP3, are summarized in the table below, which shows the percentage of LNGFR+ cells at day 6 after transfection.

| | AAV Donor Template | | | | | |
|---|---|---|---|---|---|---|
| | #3066 | | #3098 | | #3117 | |
| | AAV | RNP + | AAV | RNP + | AAV | RNP + |
| | only | AAV | only | AAV | only | AAV |
| % LNGFR+ | 2.08 | 31.2 | 1.88 | 28.9 | 0.29 | 9.43 |

Then, we studied the levels of T_{reg} associated markers in edT_{reg} cells that derived from the transfection of CD4+ cells with RNPs and one of the three AAV donor templates #3066, #3098, or #3117 (“3066edTreg,” “3098edTreg,” or “3117edTreg,” respectively). As summarized in the table below, we evaluated FOXP3 and other T_{reg} associated markers in edT_{reg} cell preparations that expressed a LNGFR selectable marker: CD4, CD25, CD127, CTLA-4, LAG3, and ICOS.

Evaluation of T_{reg} associated markers in edT_{reg} expressing LNGFR selectable marked (percentage of total cells) at day 6 after transfection.

| AAV construct | % LNGFR+/CD4− | % LNGFR−/CD4+ | % LNGFR+/CD4+ |
|---|---|---|---|
| 3066 | 0.056 | 82.2 | 17.7 |
| 3098 | 0.010 | 76.7 | 23.2 |
| 3117 | 0.021 | 90.2 | 9.69 |
| AAV construct | % LNGFR+/FOXP3− | % LNGFR−/FOXP3+ | % LNGFR+/FOXP3+ |
| 3066 | 11.4 | 3.35 | 8.98 |
| 3098 | 14.9 | 2.54 | 11.2 |
| 3117 | 4.54 | 3.61 | 7.47 |
| AAV construct | % LNGFR+/CD25− | % LNGFR−/CD25+ | % LNGFR+/CD25+ |
| 3066 | 0.25 | 72.8 | 19.1 |
| 3098 | 1.78 | 69.0 | 23.1 |
| 3117 | 0.18 | 80.0 | 10.9 |
| AAV construct | % LNGFR+/CD127− | % LNGFR−/CD127+ | % LNGFR+/CD127+ |
| 3066 | 17.7 | 0.070 | 0.39 |
| 3098 | 23.4 | 0.024 | 0.18 |
| 3117 | 9.90 | 0.094 | 0.13 |
| AAV construct | % LNGFR+/CTLA-4− | % LNGFR−/CTLA-4+ | % LNGFR+/CTLA-4+ |
| 3066 | 0.010 | 79.9 | 19.8 |
| 3098 | 0.034 | 74.4 | 25.4 |
| 3117 | 0.00521 | 88.2 | 11.5 |
| AAV construct | % LNGFR+/LAG3− | % LNGFR−/LAG3+ | % LNGFR+/LAG3+ |
| 3066 | 17.5 | 0.79 | 0.32 |
| 3098 | 22.8 | 1.07 | 0.40 |
| 3117 | 9.50 | 0.97 | 0.21 |
| AAV construct | % LNGFR+/ICOS− | % LNGFR−/ICOS+ | % LNGFR+/ICOS+ |
| 3066 | 0 | 99.8 | 0.090 |
| 3098 | 0.016 | 90.3 | 9.51 |
| 3117 | 0.038 | 82.3 | 17.5 |

These results demonstrated our ability to introduce a cis-linked clinically relevant marker for use in purification of edT_{reg} cell preparations, comprising efficient expression of LNGFR, FOXP3 and T_{reg}-associated markers for both AAV donor templates #3066 and #3117. Accordingly, either of the gene cassettes was used to introduce a cis-linked surface marker (LNGFR) for use in purification of edT_{reg} preparations.

Methods

CD4+ T cells isolated from adult healthy donors were activated with anti-CD3/CD28 beads for 48 h at cell concentration between 0.5-1 milion/ml. After an overnight rest post beads removal, cells were electroporated with human FOXP3-specific TALEN mRNAs using Neon transfection system. AAV donor templates containing MND.GFP.Knock-in with or without UCOE variants were then added to cell culture 2 h after transfection followed by a 24-hour incubation time at 30° C. After incubation, fresh media were added into culture to dilute AAV to reduce AAV-related toxicity. HDR efficiency and initial GFP expression levels was assessed by flow cytometry at day2 post editing. Cells were continued to be cultured and culture media were replenished every 2˜3 days. Aliquots of cultured cells were sampled at multiple time points during the duration of 21 days. At each time point, flow cytometry analysis was performed to examine the percentage and expression level of GFP transgene as the indication of promoter activity.

Evaluation of IL2 Cytokine Production in edT_{reg} Cells Expressing FOXP3 cDNA Cassette Either Before or after the P2A Self-Cleavage Peptide.

Results

We next studied edT_{reg} preparations to see whether they had functional activity in vitro and whether the position of a P2A self-cleavage peptide in the FOXP3 cDNA cassette had an impact on function.

We evaluated edT_{reg} cells (derived from the transfection of CD4+ cells with RNPs and one of the three AAV donor construct #3066, #3098, or #3117 (respectfully edTreg3066, edTreg3098, and edTreg3117)) expressing FOXP3 cDNA cassette either before or after the P2A self-cleavage peptide, for IL-2 cytokine activity. The intracellular IL-2 cytokine was assessed at day 3 post editing by immunostaining and flow cytometry following treated mock or edT_{reg} cells with Phorbol myristate acetate (PMA), ionomycin, and monensin (Golgi-Stop, BD Biosciences), for 5 hours at 37° C.

As shown in the results presented in the table below, we observed a reduction of IL-2 cytokine in LNGFR+ cells in edT_{reg} cells. For instance, at or about 80% of edT_{reg} cells generated using AAV donor template #3066 (“3066edTreg”) that were LNGFR− expressed IL-2, whereas only at or about 50% of the LNFGR+3066edTreg cells expressed IL-2. A similar difference was observed for the edT_{reg} cells generated using AAV donor template #3117 (“3117edTreg”), with at or about 80% of the LNGFR− 3117edT_{reg} cells expressing IL-2, and at or about 50% of the LNGFR+3117edT_{reg} cells expressing IL-2.

| Conditions | | % IL-2+ cells |
|---|---|---|
| mock | LNGFR− | 80 |
| 3066 edT_{reg} | LNGFR− | 80 |
| | LNGFR+ | 50 |
| 3117 edT_{reg} | LNGFR− | 79 |
| | LNGFR+ | 60 |
| 3098 edT_{reg} | LNGFR− | 80 |
| | LNGFR+ | 80 |

By contrast, the edT_{reg} cells generated using AAV donor template #3098 (“3098edTreg”), comprising the loss-of-function R397W mutation in FOXP3, showed no difference between both populations of LNGFR- or LNGFR+ cells, with a percentage of at or about 80% for both expressing IL-2.

Methods

Cells were plated and cultured in culture media added with 20 ng/mlPMA/DMSO (MilliporeSigma), 1 μg/ml Ionomycin (MilliporeSigma), and 1 ng/ml Monensin GolgiStop (Lifetechnologies) for 5 h at 37° C. Treated cells were then stained with surface markers including CD4 and LNGFR followed by fixation and permeabilization using BD Cytofix/Cytoperm™ Fixation/Permeabilization Solution Kit (BDB554714, BD Biosciences). Intracellular cytokines were stained with fluorochrome-conjugated anti-cytokine antibodies and analzied by FACS.

### Evaluation of SV40-Poly A and 3′-UTR Elements in AAV FOXP3 Donor Template

Results

We then compared the ability of SV40-polyA signal (“pA”) or 3 ′UTR element derived from human FOXP3 to facilitate expression of FOXP3 cDNA in edT_{regs}. The AAV donor templates #3117 and #3118 having the general structure shown below (5′- to 3′-direction) were used for this comparison. AAV #3117 comprised the MND promoter at the 5′-end of LNGFR-P2A-FOXP3 cDNA with SV40-polyA signal, while AAV #3118 comprised the MND promoter at the 5′-end of LNGFR-P2A-FOXP3 cDNA with 3′-UTR. Both AAV #3117 and AAV #3118 were flanked at both the 3′- and 5′-ends with 0.45 kb homology arms (HA):

#3117: ITR-HA-MND-LNGFR-2A-FOXP3cDNA-pA-HA-ITR,

#3118: ITR-HA-MND-LNGFR-2A-FOXP3cDNA-3′UTR-HA-ITR.

Both editing conditions led to LNGFR+ cells at comparable rates. The below table shows editing efficiency measured at Day 2 post editing based upon cis-linked LNGFR expression as assessed by flow cytometry.

| AAV donor template | % LNGFR+ cells |
|---|---|
| AAV only #1 | 0.54 |
| AAV #3117 | 19.6 |
| AAV only #2 | 0.019 |
| AAV #3118 | 22.2 |

We found that SV40-polyA achieved more stable expression of FOXP3cDNA as exemplified by the higher overall percentage of LNGFR+ cells that also were positive for a T_{reg} marker. LNGFR and the T_{reg} markers CD4, CD25, CD127, CTLA-4, LAG3, and ICOS were all expressed at comparable levels within the cell populations treated with either AAV #3117 or AAV #3118. However, the FOXP3+/LNGFR+ cells as a percentage of the total cell population was greater with AAV #3117 treatment as compared with AAV #3118 (7.47% vs. 1.27%, respectively). Intracellular cytokine staining was performed after a 5-hour treatment with PMA/Ionomycin/Golgi-Stop.

Further, intracellular IL-2 was analyzed at Day 6 post-editing. Both T cells treated with AAV donor template #3117 and those treated with AAV donor template #3118 exhibited IL-2 suppression in LNGFR+ cells. However, AAV #3117 showed a greater reduction of % IL-2+ cells within the population of LNGFR+ cells vs. the population of LNGFR− cells as compared with AAV #3118. The SV40-polyA in AAV #3117 was able to maintain stable expression of FOXP3 cDNA in edT_{reg} cells at a higher level than AAV #3118 comprising 3′-UTR under the same conditions.

| Treatment | LNGFR level | %IL-2+ cells |
|---|---|---|
| 3117edTreg | LNGFR− | 60 |
| | LNGFR+ | 78.7 |
| 3118edTreg | LNGFR− | 70.5 |
| | LNGFR+ | 75.4 |

Percentage of LNGFR+ and T_{reg} Marker Positive Cells at Day 6 after AAV Treatment

| | % LNGFR+ cells | |
|---|---|---|
| Treg marker | AAV #3117 (SV40-polyA) | AAV #3118 (3′-UTR) |
| CD4+ | 9.69 | 8.42 |
| FOXP3+ | 7.47 | 1.27 |
| CD25+ | 10.9 | 11.8 |
| CD127+ | 0.13 | 0.11 |
| CTLA-4+ | 11.5 | 13.2 |
| LAG3+ | 0.21 | 0.29 |
| ICOS+ | 9.51 | 7.59 |

Methods

RNP comprised of Cas9/T9 (1:2.5 ratio) were transfected to cells followed by deliverely of indicated AAV donor templates by AAV transduction. FACS analysis described above.

The results of these studies indicated that AAV donor template #3066 (MND-FOXP3cDNA-P2A-LNGFR flanked by 0.6 kb homology arms to FOXP3 gene) and AAV donor template #3080(MND-LNGFR-P2A flanked by 0.6 kb homology arms to FOXP3 gene) as two effective targeted donor templates in combination with Cas9/gRNA-T9 (1:2.5 ratio) RNP for edT_{reg} cell preparation and subsequent in vivo functional assessment.

### Example 15. Exemplary edT_{reg} Cell Preparation

Development of Pre-Editing CD4+ T Cell Activation and Pre-Editing Expansion Protocol

We sought to identify acceptable conditions to edit CD4+ T cells to generate edT_{reg}. Conditions tested included various activation methods: CD3/CD28 T activator beads, soluble CD3/CD28 antibodies and CD3/CD28 T expander beads, different cell concentrations (0.5 and 1 million/ml), different activation time (48, 60, 72, or 84 h), and different rest time between beads removal and editing.

Cell viability (% Live, determined by Live vs. Dead cell staining), cell activation (% CD25+), and cell numbers fold change before editing were measured for each test condition. Editing efficiency measured by % HDR shown as % GFP+ were measured at day 2 post editing.

The improved conditions for all the previously mentioned factors were identified as using the Expander beads at 3:1 bead-to-cell ratio, with a cell density of 0.5 millon cells per ml, a stimulation time of 72h and an overnight rest time before editing. These conditions led to acceptable levels of GFP expression at day 2 post editing, affording 86% cell viability, 95% CD25+ cells, and 2.3-fold cell expansion.

Culture Media Test During AAV Transduction.

Results

We then sought to identify acceptable cell media for the AAV transduction step in editing T cells to generate edT_{reg}. We tested culture media containing 5%, 10%, or 20% FBS during AAV transduction. Cells were activated and expanded in 20% FBS containing media, after editing, cells were cultured in either 5%, 10%, or 20% media before adding AAV. AAV made from multiple batches were used in the experiment.

| | | % cell viability | |
|---|---|---|---|
| Conditions | | post-editing | % LNGFR+ |
| 20% FBS | Mock | 78.5 | 6.85 |
| | 3066edTreg batch #1 | 67.2 | 28.7 |
| | 3066edTreg batch #2 | 52.1 | 28.3 |
| | 3066edTreg batch #3 | 68.8 | 35.1 |
| 10% FBS | Mock | 67.2 | 6.26 |
| | 3066edTreg batch #1 | 60.1 | 37.1 |
| | 3066edTreg batch #2 | 53.5 | 38.7 |
| | 3066edTreg batch #3 | 48.4 | 47.5 |
| 5% FBS | Mock | 44.8 | 5.35 |
| | 3066edTreg batch #1 | 41.1 | 44.6 |
| | 3066edTreg batch #2 | 35.9 | 44.7 |
| | 3066edTreg batch #3 | 38.5 | 50.5 |

Then, AAV transduction was performed in media containing either 12.5% or 20% FBS, where the SpyFi Cas9/gRNA-T9 (1:2.5) RNP was delivered into human CD4+ T cells using either Lonza nucleofector or Maxcyte followed by transduction with AAV6 donor template #3066. At 24 h post editing, media containing 20% FBS were used to dilute cell culture. Cell viability (Live vs. Dead cell staining) and HDR efficiency (LNGFR staining) were determined by flow cytometry at day 2 post editing. The results of these experiments are shown in below. The use of 12.5% FBS during AAV transduction enhanced editing efficiency without compromising viability and also demonstrated the similar editing efficiency using both the Lonza and Maxcyte electroporation instruments.

Reduction of FBS during AAV transduction led to enhanced editing efficiency. However, low levels of FBS had a negative impact on cell viability. Based on these studies, the use of 12.5% FBS during AAV transduction enhanced editing efficiency without compromising viability post-editing leading to acceptable levels of edT_{reg} production.

% Cell Viability after Varying Electroporation/Nucleofection Conditions

| | Electroporation/nucleofection | | | |
|---|---|---|---|---|
| | % FBS | mock | edT_{reg} prep #1 | edT_{reg} prep #2 |
| Lonza | 12.5 | 91.4, 79.1 | 83.7 | 61.6 |
| | 20.0 | N/A | 82.2 | N/A |
| Maxcyte | 12.5 | 91.4, 82.2 | 78.5 | 61.3 |
| | 20.0 | N/A | 73.5 | 66.3 |

% LNGFR+ after Varying Electroporation/Nucleofection Conditions

| | Electroporation/nucleofection | | | |
|---|---|---|---|---|
| | % FBS | mock | edT_{reg} prep #1 | edT_{reg} prep #2 |
| Lonza | 12.5 | 4.4, 3.9 | 29.1 | 26.2 |
| | 20.0 | N/A | 20 | N/A |
| Maxcyte | 12.5 | 7.3, 5.1 | 31.9 | 33.4 |
| | 20.0 | N/A | 23.8 | 26.3 |

Tests of Different Electroporation Conditions for the Generation of edT_{reg} with Lonza Nucleofection.

We then performed extensive analysis of alternative nucleofection programs for CD4+ T cell editing to generate edT_{reg}. Use of either the Lonza EO-115 or DN-102 programs achieved high rates of HDR editing efficiency while maintaining post editing viability. AAV donor templates 3066 and 3080 were used in the experiment.

Exemplary edT_{reg} Cell Production Protocol.

We established an exemplary protocol for edT_{reg} production. The list of reagents and detailed culture conditions for this protocol are shown in the table below.

| Action | Details |
|---|---|
| CD4 isolation | Freshly isolated CD4+ T cells from frozen PBMC (Easysep CD4 |
| | negative isolation #19052) |
| CD4 activation | 0.5 million/ml with 3:1 bead-to-cell using Dynabeads ™ Human T- |
| | Expander CD3/CD28 (ThermoFisher 11141D) |
| Culture media | RPMI1640 with 20% FBS, HEPES, GLUTAMAX, β-mercaptoethanol, |
| | IL-2 (50 ng/ml each) |
| nuclease | Aldevron SpyFi Cas9 (research grade):Biospring T9 guide at 1:2.5 |
| | molar ratio (20 pmol:50 pmol) |
| RNP delivery | Maxcyte, Lonza, or Neon transfection systems (Mock: treated with Cas9 |
| | only and AAV) |
| AAV | AAV6, add at 20% v/v of cell culture media after RNP delivery |
| Culture media | RPMI1640 with 12.5% FBS, HEPES, GLUTAMAX, β- |
| during AAV | mercaptoethanol, IL-2 (50 ng/ml each) |
| transfection | |
| enrichment | CD271 Microbead (Miltenyi #130-099-023), LS column (Miltenyi |
| | #130-042-401) |
| expansion | Expand mock and enriched edT_{reg} cells with 3:1 T-expander beads in G- |
| | Rex ® 6-well or G-Rex ® 10 |
| Expansion | RPMI1640 with 20% FBS, HEPES, GLUTAMAX, β-mercaptoethanol, |
| media | IL-2 (100 ng/ml each), rapamycin (100 nM, one-time treatment when |
| | expanding); half media change 3 days and 5 days after culture; no |
| | additional beads or rapamycin added during the 7-day expansion |
| cryopreservation | Remove beads and freeze mock or enriched edT_{reg} in Cryostor CS10 at |
| | end of expansion |

The 14-day production timeline and protocol are shown in the table below.

| Day | Action |
|---|---|
| 0 | Thaw PBMC, CD4+ isolation, beads stimulation |
| 3 | Remove beads |
| 4 | Genome editing with RNP and AAV delivery |
| 5 | AAV dilution (1x volume media) |
| 6 | Check editing rate |
| 7 | Enrich, expand in G-Rex with beads and rapamycin |
| 10 | Change ½ media |
| 12 | Change ½ media |
| 14 | Remove beads, cryopreservation, phenotyping |

Efficient Enrichment for HDR Gene-Edited edT_{reg} Using LNGFR (CD271) Microbeads and magnetic column separation.

Results

We then sought to find an efficient enrichment method for HDR gene-edited edT_{reg}, expressing LNGFR.

We edited CD4+ T cells with the AAV #3066 construct, following the protocol presented in the previous section. The resulting 3066edTreg, expressing the LNGFR marker (i.e. LNGFR+ cells), were purified (enriched) using LNGFR (CD271) microbeads and magnetic column separation 3 days after cell editing, and subjected to a cell expansion period of 7 days post-enrichment.

A LNGFR staining experiment on edT_{reg} cells, post-microbead purification, showed 99.2% of the purified cells were expressing LNGFR, as compared with 0.07% of mock edited cells. The average purity of the LNGFR+edT_{reg} cell preparation from 6 experiments was 98.6%. The average number of 3066edTegs in expanded cell composition from 6 experiments showed expansion at an average of 60-fold during the 7-day culture in G-Rex® (WilsonWolf, St. Paul, Minn. USA).

Moreover, these LNGFR+ cells expressed FOXP3 and other T_{reg} markers including CD4, CD25, CTLA-4, ICOS, and LAG3, and showed reduced of IL-2, TNFa and IFNg compared to LNGFR− cells as shown in the table below.

| | | % Cytokine-Positive Cells | | |
|---|---|---|---|---|
| Conditions | | IL2+ | TNFa+ | IFNg+ |
| Mock edited | LNGFR− | 36.1 | 39.6 | 10.5 |
| 3066 edT_{reg} | LNGFR+ | 6.7 | 10.0 | 4.4 |
| | LNGFR− | 50.1 | 33.7 | 8.6 |

We were able to generate large numbers of highly purified edT_{reg} cell preparations using our editing and cell expansion protocols based upon the methods developed. Our purified and expanded edT_{reg} preparations expressed high levels of FOXP3 and LNGFR as well as CD25, CTLA-4 and ICOS and low levels of CD127, which were consistent with a T_{reg}-like phenotype. Expression of FOXP3cDNA also led to reduced expression of pro-inflammatory cytokines (IL-2, TNFalpha, and IFNgamma) as assessed by the response to PMA/ionomycin stimulation.

Methods

Human primary CD4+ T cells were enriched at 3 days after gene editing with the AAV #3066 donor construct, using LNGFR (CD271) microbeads (Miltenyi #130-099-023) and magnetic column separation (Miltenyi #130-042-401). Enriched cells were mixed with CD3/CD28 T-expander beads at 3:1 bead-to cell ratio in the T cell expansion media (RPMI1640, 20% FBS, HEPES, GLUTAMAX, β-Mercaptoethanol, IL-2 (100 ng/ml), and 100 nM rapamycin. Cell cultures were placed in G-Rex® (6 well, WilsonWolf, St. Paul, Minn. USA) plated at 1.5˜2 million per well for 7 days. At days 3 and 5 of culture in G-Rex®, one-half volume of media was replenished. At the end of the 7-day expansion in G-Rex®, cell count, purity, and phenotypes were analyzed.

### Example 16. Generation of Expanded tT_{reg} for Comparison Studies

In evaluation of edT_{reg} preparations, we used tT_{reg} (thymic T_{reg}), also known as nT_{reg} (natural T_{reg}), as a control in our experiments. There were several published protocols for ex vivo tT_{reg} expansion, however, they were significantly different from our edT_{reg} protocol in terms of isolation, expansion condition, and duration.

tT_{reg} Expansion Protocol

We developed a tT_{reg} expansion protocol, described below, that closely matched the in vitro culture and handling of edT_{reg}. The reagents and conditions used in tT_{reg} expansion are summarized in the following table.

| Action | Details |
|---|---|
| tT_{reg} isolation | EasySep ™ Human CD4+CD127lowCD25+ Regulatory T Cell isolation |
| | kit (#18063) |
| Initial expansion | 0.5 million/ml in plate, with 3:1 bead-to-cell using Dynabeads ™ Human |
| | T-Expander CD3/CD28 (ThermoFisher 11141D), beads removed at day |
| | 3 |
| G-Rex ® | Plate in G-Rex ® at day 7 with Dynabeads ™ Human T-Expander beads |
| expansion | (3:1) |
| Expansion | RPMI1640 with 20% FBS, HEPES, GLUTAMAX, β-mercaptoethanol, |
| media | IL-2 (100 ng/ml each), rapamycin (100 nM, one-time treatment when |
| | expanding); half media change 2-3 days; G-Rex ® expand for 7 days |
| cryopreservation | freeze mock or enriched tT_{reg} in Cryostor CS10 |

The 14-day production timeline and protocol for tT_{regs} are shown in the table below.

| Day | Action |
|---|---|
| 0 | Thaw PBMC, tT_{reg} isolation, beads stimulation/expansion |
| 3 | Remove beads, add 1x volume media |
| 5 | Add 1x volume media |
| 7 | expand in G-Rex ® with beads and rapamycin |
| 9-12 | Change ½ media every 2-3 days, count cells every other |
| | media change |
| 13 | Phenotyping, determine purity |
| 14 | Remove beads, cryopreservation |

In Vitro Characterization of tT_{reg} Generated with edT_{reg}-Matching Protocol.

Results

Using this edT_{reg}-matching protocol for the generation of tT_{reg} cells, we first assessed the FOXP3 expression in tT_{reg} derived from 4 different donors, using conventional CD4 cells (“Tconv” or “cony CD4”), which are CD4+CD25− cells that were expanded and stained/analyzed in parallel as positive control.

| | % CD4+/FOXP3+ cells |
|---|---|
| Conventional CD4 | 78 |
| tT_{reg} | 9 |

Average of Four Experiments Each

The FOXP3 expression in the tT_{reg} cells and the Tconv cells were evaluated. The table below summarizes purity and cell expansion from an average of four experiments, which shows an average of 77.5% of the total cells expressing FOXP3 and therefore being tT_{reg} cells, whereas only 10% of Tconv cells expressed FOXP3.

| Total tT_{reg} cells | % FOXP3+ | Total FOXP3+ cells |
|---|---|---|
| 1.82 × 108 | 77.5 | 1.40 × 108 |

Methods

Natural T_{reg} cells were isolasted from healthy donor PBMC using a T_{reg} isolation kit (Stemcell), then activated with CD3/CD28 T-expander beads at 3:1 bead-to-cell ratio for initial expansion. T_{reg} cells were cultured at 0.5 million/ml for 72 hours in the presence of beads, and additional 96 hours culture in the absence of beads. tT_{reg} cells were subsequently plated into Grex 6-well culture vessel CD3/CD28 T-expander beads at a 3:1 bead-to-cell ratio with expansion media. Expansion media was replenished every 2˜3 days during the 7-day culture in Grex. Cells were separated from beads 7 days after culture by magnetic separation and cryopreserved in the cyrostor CS10 media in liquid nitrogen cabinet.

### Example 17. Generation of edT_{reg} Following Editing of Alternative Target Loci in Human T Cells

Results

To determine if we can achieve the generation of edT_{reg} following editing of alternative target loci in human primary T cells, we compared expression levels of FOXP3 following editing at FOXP3 vs. AAVS1 loci. Similar, high-levels of HDR editing were achieved at both FOXP3 and AAVS1 locus, two days after editing, when compared to unedited cells and cells transfect with donor templates only. The percentage of cells expressing the transgenic markers (GFP or LNGFR) is summarized in the table below.

Percentage of cells expressing GFP or LNGFR
after editing the FOXP3 or AAVS1 locus.

| Locus | Donor template | Marker | AAV only | AAV and RNP |
|---|---|---|---|---|
| FOXP3 | AAV-MND.GFP.polyA | GFP | 6.89% | 77.5% |
| FOXP3 | AAV-MND.FOXP3cDNA.LNGFR.polyA | LNGFR | 1.43% | 37.3% |
| AAVS1 | AAV-MND.GFP.polyA | GFP | 2.80% | 74.8% |
| AAVS1 | AAV-MND.FOXP3cDNA.LNGFR.polyA | LNGFR | 0.58% | 28.7% |

Moreover, our data suggested that AAVS1 could be used as the alternate locus to express the FOXP3 transgene to generate edT_{reg}. Notably, MND-mediated, transgene expression at the edited FOXP3 locus was higher than expression observed for the AAVS1 locus, for both donor templates tested. This difference may facilitate distinct levels of FOXP3 expression in edT_{reg} preparations permitting alternative uses for cells edited at these loci.

Methods

Cas9/gRNA-T9 or Cas9/gRNA-N2 RNP complex was electroporated into activated CD4+ T cells to generate DNA double-strand break at FOXP3 or AAVS1 locus, respectively, followed by AAV-mediated donor template delivery for homology directed repair. The donor templates contained either MND-GFP.polyA or MND-FOXP3cDNA.P2A.LNGFR.polyA gene expression cassettes flanked by locus-specific homology arms. At day 2, editing efficiency was measured by flow cytometry to determine percentage GFP+ or LNGFR+. The methods described in Example 15 for CD4 cell activation, editing and FACS were used.

### Example 18. In Vitro Functional Characterization of edT_{reg} Preparations

Results

To quantify on-target integration of donor HDR cassettes following edTreg productions, we sought to develop a droplet digital PCR (ddPCR) assay. We designed ddPCR primers along with HEX or FAM probes to quantify the presence of the LNGFR, or HDR editing rate, in edT_{reg} generated using the AAV donor template #3066. The HDR editing rate as measured by ddPCR would then be compared to the HDR editing level as previously measured by cell staining and flow-cytometry.

The ddPCR data correlates directly with HDR editing rates as determined using flow cytometry to track protein expression. This assay should permit molecular characterization of edT_{reg} preparations, including those that lack relevant protein markers and/or eliminate the need to track FOXP3 expression in edT_{reg} preparations using intracellular staining. This assay also provides a potential useful release criteria for edT_{reg} preparations.

Methods

Edited cells were enriched using LNGFR-antibody column separation (Miltenyi). Both enriched and flow through preparations were then expanded separately for 7-day in G-Rex® (WilsonWolf, St. Paul, Minn. USA). The LNGFR-enriched cells were at or about 90% LNGFR+ and the expanded flow through cells were at or about 1% LNGFR+. Portion of enriched cells and flow through were then mixed to generate cell preparation with 70% LNGFR+ purity. Cell samples were analyzed by flow cytometry as well as ddPCR to detect percentage of LNGFR+ and on-target gene integration.

For ddPCR, genomic DNA isolated from each sample was set up to generate droplet and then PCR amplified in reactions containing primer mixture indicated in the tables below. Data analysis is performed using Quant soft. Percent HDR is the ratio of the insert concentration to the control concentration.

| Primer mix | | |
|---|---|---|
| for insert | 974 bp | FAM |
| Forward | GGCACCTCCAGAACAAGACC (SEQ ID NO: 129) | |
| Reverse | TCCTGATCCTCACTGTTCTGTGTC (SEQ ID NO: 130) | |
| Probe-FAM | AGACCCACAACCACAGCAGC (SEQ ID NO: 131) | |
| Primer mix for | | |
| control | 976 bp | HEX |

| | |
|---|---|
| Forward | GTTCACACGCATGTTTGCCT (SEQ ID NO: 132) |
| Reverse | ATCCTGAGGGTACTGACGCT (SEQ ID NO: 133) |
| Probe-Hex | TGGCGGTGACTGGGATGGC (SEQ ID NO: 134) |

### Example 19. In Vivo Functional Characterization of edT_{reg} Preparations

We next evaluated the in vivo functional activity of the edT_{reg} preparations derived from our production protocol.

Evaluation of In Vivo Function of edT_{reg} Processed with Different Approaches.

Results

We found that edT_{reg} was purified efficiently using a clinically relevant surface marker, and that they were effectively expanded and cryopreserved. The resulting cell preparations functioned efficiently in vivo to block Xenogeneic Graft-versus-Host-Disease (xenoGVHD) in mice (also known as the CD4 Adoptive Transfer Inflammation (CATI) mouse model.) Similar results were observed when using: cryopreserved compared with freshly generated edT_{reg}; LNGFR+ cells enriched by either FACS or column separation; and both LNGFR knock-in (KI) and GFP knock-in edT_{reg}, thus demonstrating that the method of editing a genome of a lymphocytic cell was robust and did not depend on specific protocols for preparing effective cell compositions.

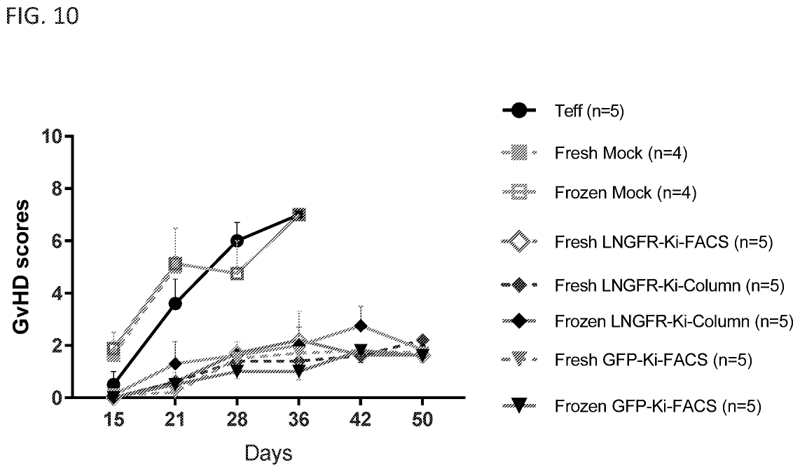

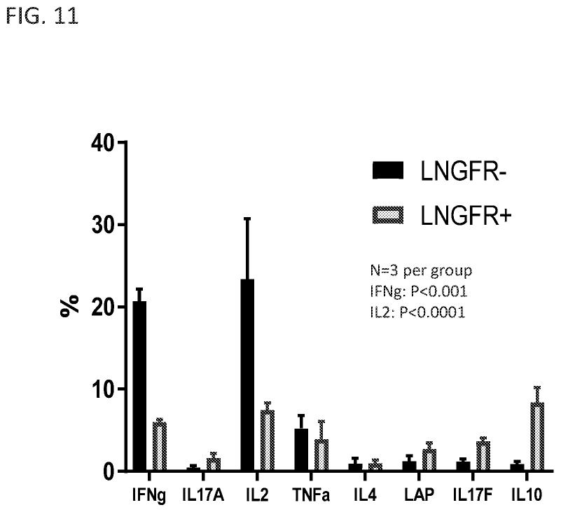

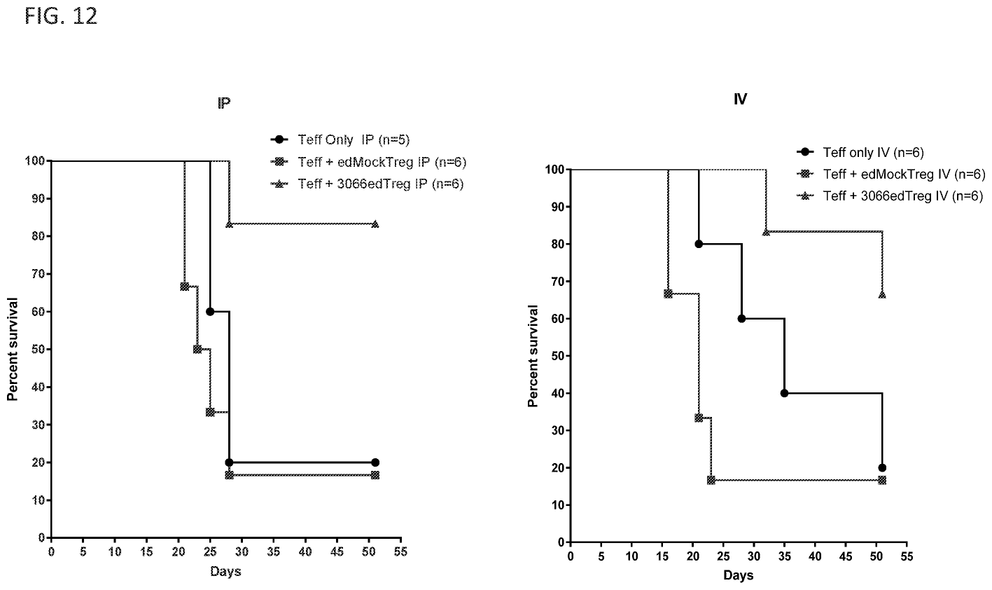

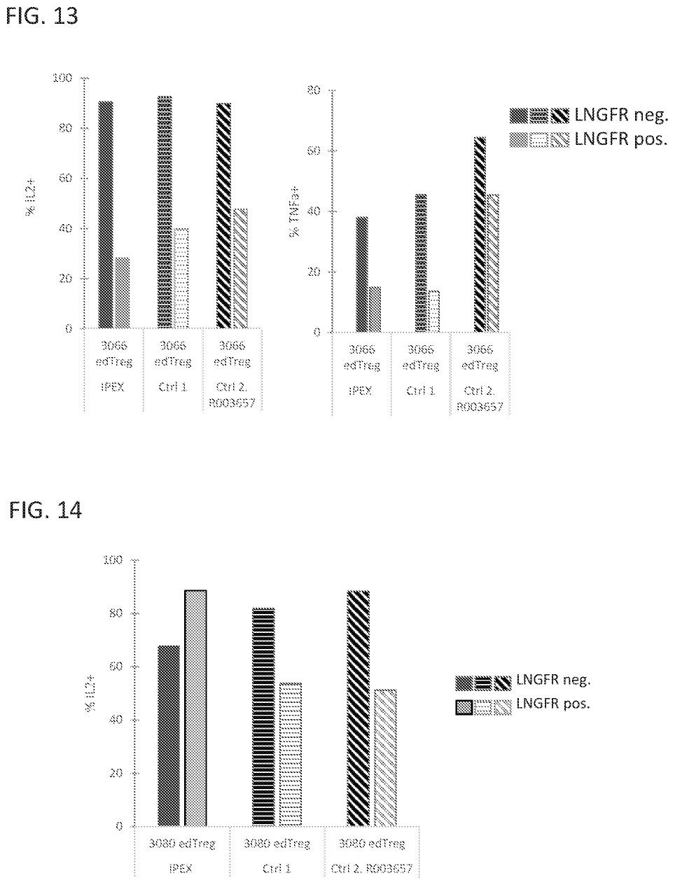

For the in vivo xenoGVHD study, the edT_{reg} cells used either expressed GFP or LNGFR marker, and endogenous FOXP3, and the GFP or LNGFR expression appeared similar for fresh versus freeze/thaw preparations. Survival rates of 60% to 100% after 50 days were observed for both frozen and fresh cell preparations, with no significant difference between the frozen and the fresh cell preparations. FIG. 10 shows GVHD scores over the course of 50 days.

Therefore, the cryopreserved edT_{reg} cell preparations showed similar capability in suppressing xenoGVHD compared with freshly generated edT_{reg}. The results suggested that the enrichment of LNGFR+ edT_{reg} by either FACS or column separation performed similarly to FACS-enriched GFP+ edT_{reg}, and that both LNGFR knock-in (KI) and GFP knock-in edT_{reg} effectively suppressed xenoGVHD, as shown in FIG. 10. Thus, edT_{reg} generated using our approach could be purified efficiently using a clinically relevant surface marker, expanded and cryopreserved. Preparations handled in this manner proved to function efficiently in vivo to inhibit clinical aspects of xenoGVHD in this mouse model.

Methods

In the condition where cryopreserved cells need to bed used, cells were resuspended in Cryostor CS10 (BioLife Solutions) freezing media at 5˜100 million cells/ml and aliquoted to cryovials at 1 mL per vial. Vials placed in a cryocontainer such as CoolCell (BioCision) or Mr. Frosty (Thermo Fisher) were transferred from room temperature to a −80° C. freezer to allow temperature reduction rate to be approximately 1° C./min. approximately 4˜96 h in −80° C. freezers, cryovials were then transferred to liquid nitrogen cabinet for storage. 8-10 weeks old male NSG (NOD-scid IL2Rgamma-nul, Jackson Laboratory) mice were exposed to whole body irradiation at 200cgy prior to I.V. infusion of edT_{reg}, mock-edited or in some case, tT_{reg} at 8×10⁶ cells/mouse. In some study groups, mice were only treated with irradation. Bodyweight of each study subject was measured and recorded as initial bodyweight. Three days after infusion, each mouse in the study chort were adminstered with 4×10⁶ autologous CD4 effector T cells freshly isolated from cryopreserved PBMC through tail vein I.V. injection. Change in bodyweight was monitored 2˜3 times each week and GvHD socores were assessed weekly for approximately 50˜65 days after effector T cells injection. GvHD scores were assessed according to bodyweight change, posture, activity, fur texture, and skin integrity. A score between 0-2 was given for each category at the interval of 0.5 and the total scores were recored. When bodyweigt loss is great than 20% of the initial bodyweight, the mouse is humanly euthanized as study end point.

Persistence of edT_{reg} In Vivo in the xenoGVHD Model.

Results

edT_{reg} in vivo showed persistence in the xenoGVHD mouse model. LNGFR+FOXP3+ edT_{reg} were detected in mice at 90 days post-adoptive transfer and upon stimulation, the LNGFR+ cells produced a lower level of inflammatory cytokines (IL-2, TNFalpha, IFNgamma) than the LNGFR− T cells as shown below. These results demonstrated long-term maintenance of edT_{reg} function and phenotype in vivo. Results of FOXP3, CTLA-4, CD25, and CD127, along with LNGFR staining are shown in the table below.

| | % CD4+ | % LNGFR+ | % LNGFR+ | % LNGFR+ | % LNGFR+ |
|---|---|---|---|---|---|
| Mouse | CD45RO+ | FOXP3+ | CTLA4+ | CD25+ | CD127+ |
| #1 | 47.1 | 0.53 | 0.26 | 0.20 | 0.045 |
| #2 | 53.9 | 1.2 | 0.44 | 0.49 | 0.14 |
| #3 | 61.0 | 0.44 | 0.15 | 0.18 | 0.012 |

LNGFR⁺FOXP3⁺ edT_{reg} were detected in mice at 90 days post-adoptive transfer indicating that edT_{reg} persisted and maintained a regulatory T cell phenotype. Upon stimulation, the LNGFR⁺ cells produced a lower level of inflammatory cytokines (IL-2, TNFα, IFNg) than the LNGFR⁻ T cells.

Methods

At 90 days post cell transfer into the xenoGVHD model, spleens from 3 mice that received human edT_{reg} and T_{eff} were collected to examine for the presence and immunophenotypes of long-term engrafted edT_{reg}. Human CD4+T populations identified as hCD45RO+CD4+ or hCD3+CD4+ were analyzed for LNGFR, T_{reg}-markers and intracellular cytokines by flow cytometry. For cytokine production in response to stimulation, cells were treated with PMA/ionomycin and Golgi-stop for 5h at 37 C before staining for the indicated cytokines. Spleens collected from mice were gently meshed in PBS buffer to obtain cell suspension. Splenic cells were treated with ACK (Ammonium-Chloride-Potassium) lysis buffer to remove red blood cells before immunostaining. Intracellular markes were stained using True Nuclear transcription factor staining buffer set. For cytokine production analysis, cells were cultured in culture media supplemented with PMA/Ionomycin and Golgi-stop for 5 h at 37° C. before immuno-staining using BD cytofix/cytoperm Fixation/Permeabilization Solution Kit

### Example 20: Editing Genome of T Cells from IPEX Subjects

Results

To evaluate the potential for edT_{regs} as a T cell therapy for IPEX subjects, we edited CD4+ T cells from an IPEX subject having I363V FOXP3 mutation, or control cells derived either from healthy donor cord blood or healthy donor PBMC, with SpyFiCas9/gRNA T9 (1:2.5 ratio) RNP prepared according to Example 15 in combination with AAV donor template #3080 or AAV donor template #3066. As indicated in previous sections, AAV donor template #3066 had the following construct structure:

ITR-(0.6 kb HA for T9)-MND-FOXP3cDNA-P2A-LNGFR-pA-(0.6 kb HA for T9)-ITR,

while AAV donor template #3080 had the following construct structure:

ITR-(0.6 kb HA for T9)-MND-LNGFR-P2A-FOXP3exon1-pA-(0.6 kb HA for T9)-ITR.

Expression of full length FOXP3cDNA via HDR-editing restored a T_{reg} phenotype to T cells derived from an IPEX subject, demonstrating the potential of this approach as a T cell therapy for IPEX.

Expression of functional WT FOXP3 cDNA either from the endogenous WT locus or via introduction of a WT FOXP3 cDNA was required to effect the T_{reg}-like phenotype in T cells derived from the healthy donor. The control edT_{reg} cells generated from CD4+ T cells from either healthy donor cord blood or PBMC afforded decreased levels of inflammatory cytokines IL2 and TNFα in the LNGFR+ cells. These results were effected with AAV donor template #3066 encoding full length wild-type FOXP3 and with AAV donor template #3080 comprising only the FOXP3 1^{st} coding exon.

In the case of T cells derived from an IPEX subject, inclusion of a WT FOXP3cDNA in the donor template was required to restore a T_{reg} phenotype. AAV donor template #3066 encoding full length wild-type FOXP3 effected reduction in the percentage of IL2+LNGFR+ cells, but AAV donor template #3080 did not achieve a comparable result.

In addition, IPEX edT_{reg} cells were enriched to a highly pure population using LNGFR selection marker (see tables below) and the LNGFR-enriched IPEX edT_{reg} expressing WT FOXP3 cDNA displayed a phenotype and cytokine profile similar to that of control edTreg cells.

% LNGFR+

| Cell source | Edited Cells | Enriched | Flow through |
|---|---|---|---|
| IPEX | 3066 edTreg | 99 | 2 |
| | 3080 edTreg | 98.9 | 4.6 |
| Healthy control | 3066 edTreg | 98.7 | 3.3 |
| #1 (cord blood) | 3080 edTreg | 99.6 | 6.0 |
| Healthy control | 3066 edTreg | 99.2 | 3.1 |
| #2 (PBMC) | 3080 edTreg | 99.3 | 4.3 |

Percentage of cells with high indicated
cytokine levels in IPEX edT_{reg}

| | % IL2+ | % TNFα+ | % IFNγ+ |
|---|---|---|---|
| Mock (LNGFR−) | 30.4 | 28.2 | 32.1 |
| 3066edTreg | 2.5 | 2.2 | 5.9 |
| (LNGFR+) | | | |
| 3066edTreg | 40.8 | 40.6 | 39.7 |
| (LNGFR−) | | | |

Percentage of cells with high indicated
cytokine levels in control edT_{reg}

| | % IL2+ | % TNFα+ | % IFNγ+ |
|---|---|---|---|
| Mock (LNGFR−) | 45.3 | 50.1 | 40.3 |
| 3066edTreg | 3.2 | 8.0 | 10 |
| (LNGFR+) | | | |
| 3066edTreg | 40.9 | 23 | 24.5 |
| (LNGFR−) | | | |

Methods

For each of the evaluated AAV donor templates, T cells were isolated from cord blood of an IPEX subject having I363V mutation. In parallel, control 1 (Ctrl 1) T cells were isolated from healthy cord blood and control 2 (Ctrl 2) T cells were from healthy adult PBMC. The T cells were each treated according to the protocol described in Example 15 using SpyFiCas9/gRNA-T9 (1:2.5 ratio) RNP and AAV donor template #3066. FACS analysis of each T cell preparation was performed at day 2 post-editing.

| | % LNGFR+ cells after indicated treatment | | |
|---|---|---|---|
| Cell source | mock | 3066edTreg | 3088edTreg |
| IPEX | 3.4 | 27.6 | 39.5 |
| Control #1 | 2.9 | 26.3 | 45.9 |
| Control #2 | 1.7 | 20.1 | 38.3 |

### Example 21: In Vitro Suppression Assay

We used two alternative proliferation dye-based in vitro suppression assays to determine whether the edT_{reg} generated using the exemplary editing protocol of Example 10 were able to suppress proliferation of CD4 T_{eff} in response to CD3/CD28 stimulation.

In Method 1, edT_{reg} or mock edited T cells were mock-irradiated or irradiated with 3000 rad. Separately, Teff, bulk CD4, derived from autologous CD4+ cells isolated from PBMCs, were prepared, with Teff labelled with CellTrace proliferation dye. The Teff cells and edited T cells were mixed at different ratios, and stimulated with anti-CD3/CD28 beads at 1:32 ratio. The remaining CellTrace dye in Teff was analyzed by flow cytometry after the 96 hour-incubation to evaluate the proliferation of Teff. Negative control was Tcon only with no beads. Positive control was Tcon only with 1:32 beads.

Method 2 was similar in protocol to Method 1, only proliferation was determined 72 hours post incubation using dye dilution. Further, in Method 2, no irradiation of input edTreg or mock cells was performed.

Percent suppression for both methods was calculated using the following formula:

% suppression=(% proliferation_{w/o suppressor}−% proliferation_{w/ suppressor})/(% proliferation_{w/o suppressor})×100.

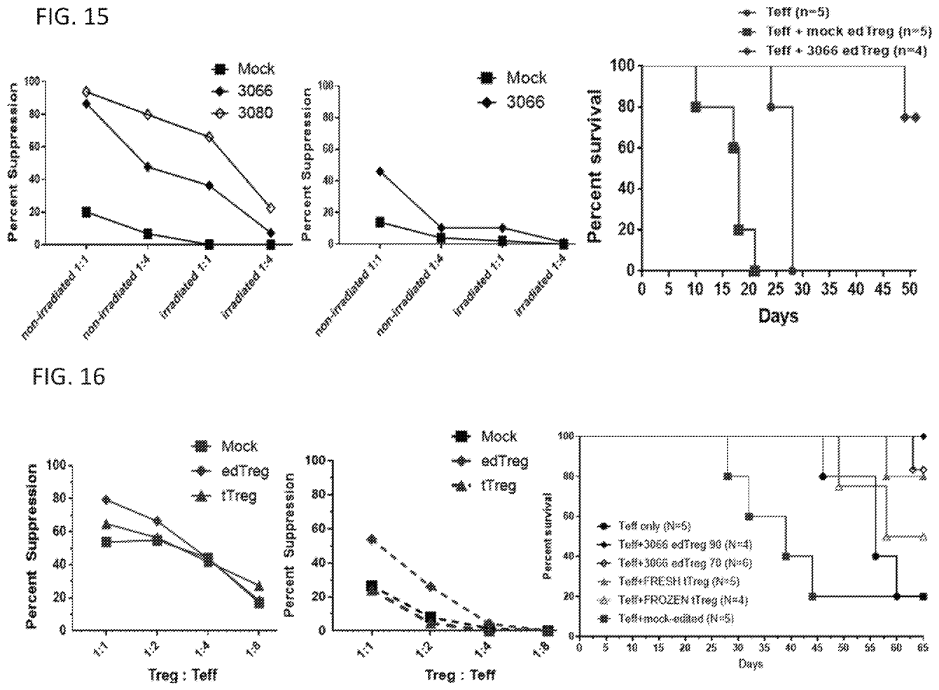

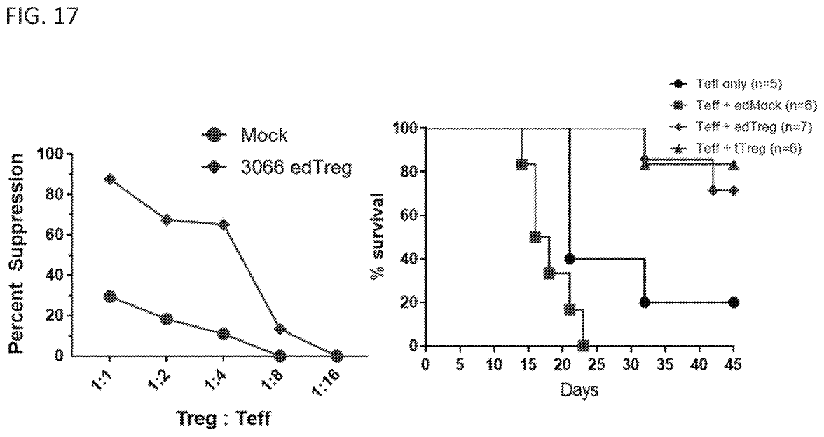

Our results indicated that the edT_{reg} generated from SpyFi Cas9/gRNA-T9 and AAV donor template #3066 (MND-FOXP3 cDNA-P2A-LNGFR flanked by 0.6 kb homology arms to FOXP3) or #3080 (MND-LNGFR-P2A-FOXP3 cDNA flanked by 0.6 kb homology arms to FOXP3) were able to suppress Teff proliferation in vitro (FIGS. 15-17). An additional key negative control—inclusion of mock edited cells—was used in our assays. This control may be important as these cells can compete for IL2 and potentially exhibit suppressive activity. Our data demonstrated that the edT_{reg} exhibited suppressive activity that is significantly greater than mock cells. FIGS. 15-17 show in vitro and in vivo results of edT_{reg}-mediated suppression assays from three different batches of edT_{regs}. The in vitro results of Method 1 protocol evaluating T_{eff} proliferation suppression by edT_{regs} corresponded to the in vivo results from the same edT_{reg} batch generated from #3066. FIGS. 16-17 show in vitro results of Method 2 protocol evaluating T_{eff} proliferation suppression by edT_{regs}, and corresponding in vivo results from the same edT_{reg} batch generated from #3066.

The corresponding in vivo results from the murine CATI model described in Example 13 are summarized below. Each of the three batches of edT_{reg} arising from AAV donor template #3066 afforded inhibition of T_{eff} suppression in the mouse model, thus leading to an increased survival of the mouse cohort treated with edT_{reg}. The three edT_{reg} compositions exhibited immunosuppressive function in vitro and in vivo, and the functional immunosuppressive activity was comparable to natural T_{reg} evaluated in parallel (see, FIGS. 16-17).

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications within the true scope and spirit of the invention.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

### Sequences

In addition to sequences disclosed elsewhere in the present disclosures, the following sequences are provided as they are mentioned or used in various exemplary embodiments of the disclosures, which are provided for the purpose of illustration. SEQ ID NOS:141-162 include AAV donor template sequences.

| SEQ ID | | |
|---|---|---|
| NO | Feature | Sequence |
| 1 | T1 spacer | TTCCAGGGCCGAGATCTTCG |
| | targeting human | |
| | FOXP3 | |
| 2 | T3 spacer | CGCCTCGAAGATCTCGGCCC |
| | targeting human | |
| | FOXP3 | |
| 3 | T4 spacer | TCGAAGATCTCGGCCCTGGA |
| | targeting human | |
| | FOXP3 | |
| 4 | T7 spacer | GGCCCTGGAAGGTTCCCCCT |
| | targeting human | |
| | FOXP3 | |
| 5 | T9 spacer | TCCAGCTGGGCGAGGCTCCT |
| | targeting human | |
| | FOXP3 | |
| 6 | T18 spacer | TCAGACCTGCTGGGGGCCCG |
| | targeting human | |
| | FOXP3 | |
| 7 | R1 spacer | GAGCCCCGCCTCGAAGATCT |
| | targeting human | |
| | FOXP3 | |
| 8 | PAM sequence | AGG |
| 9 | PAM sequence | TGG |
| 10 | PAM sequence | AGG |
| 11 | PAM sequence | GGG |
| 12 | PAM sequence | GGG |
| 13 | PAM sequence | GGG |
| 14 | PAM sequence | CGG |
| 15 | P1 spacer | ATTCCCAGGGCCGGTTAATG |
| | targeting human | |
| | AAVS1 | |
| 16 | P3 spacer | GTCCCCTCCACCCCACAGTG |
| | targeting human | |
| | AAVS1 | |
| 17 | P4 spacer | ACCCCACAGTGGGGCCACTA |
| | targeting human | |
| | AAVS1 | |
| 18 | N1 spacer | CCTCTAAGGTTTGCTTACGA |
| | targeting human | |
| | AAVS1 | |
| 19 | N2 spacer | TATAAGGTGGTCCCAGCTCG |
| | targeting human | |
| | AAVS1 | |
| 20 | N3 spacer | CCATCGTAAGCAAACCTTAG |
| | targeting human | |
| | AAVS1 | |
| 21 | PAM sequence | TGG |
| 22 | PAM sequence | GGG |
| 23 | PAM sequence | GGG |
| 24 | PAM sequence | TGG |
| 25 | PAM sequence | GGG |
| 26 | PAM sequence | AGG |
| 27 | mT20 spacer | GACTCCTGGGGATGGGCCAA |
| | target murine | |
| | FOXP3 | |
| 28 | mT22 spacer | TTGGCCCTTGGCCCATCCCC |
| | target murine | |
| | FOXP3 | |
| 29 | mT23 spacer | CCAGCTTGGCAAGACTCCTG |
| | target murine | |
| | FOXP3 | |
| 30 | PAM sequence | GGG |
| 31 | PAM sequence | AGG |
| 32 | PAM sequence | GGG |
| 33 | human TRAC | ACAAAACTGTGCTAGACATG |
| | spacer sequence | |
| | G2 | |
| 34 | human TRAC | TCAAGAGCAACAGTGCTG |
| | spacer sequence | |
| | G4 | |
| 35 | PAM sequence | AGG |
| 36 | PAM sequence | TGG |
| 37 | FOXP3cDNA- | GCCACCATGCCTAATCCTCGGCCTGGAAAGCCTAGCGCTCCTTCTCTTGCT |
| | P2A-LNGFR | CTGGGACCTTCTCCTGGCGCCTCTCCATCTTGGAGAGCCGCTCCTAAAGC |
| | | CAGCGATCTGCTGGGAGCTAGAGGACCTGGCGGCACATTTCAGGGCAGA |
| | | GATCTTAGAGGCGGAGCCCACGCTAGCTCCTCCAGCCTTAATCCTATGCC |
| | | TCCTAGCCAGCTCCAGCTGCCTACACTGCCTCTGGTTATGGTGGCTCCTAG |
| | | CGGAGCTAGACTGGGCCCTCTGCCTCATCTGCAAGCTCTGCTGCAGGACA |
| | | GACCCCACTTCATGCACCAGCTGAGCACCGTGGATGCCCACGCAAGAAC |
| | | ACCTGTGCTGCAGGTTCACCCTCTGGAATCCCCAGCCATGATCAGCCTGA |
| | | CACCTCCAACAACAGCCACCGGCGTGTTCAGCCTGAAAGCCAGACCTGG |
| | | ACTGCCTCCTGGCATCAATGTGGCCAGCCTGGAATGGGTGTCCAGAGAAC |
| | | CTGCTCTGCTGTGCACATTCCCCAATCCAAGCGCTCCCAGAAAGGACAGC |
| | | ACACTGTCTGCCGTGCCTCAGAGCAGCTATCCCCTGCTTGCTAACGGCGT |
| | | GTGCAAGTGGCCTGGATGCGAGAAGGTGTTCGAGGAACCCGAGGACTTC |
| | | CTGAAGCACTGCCAGGCCGATCATCTGCTGGACGAGAAAGGCAGAGCCC |
| | | AGTGTCTGCTCCAGCGCGAGATGGTGCAGTCTCTGGAACAGCAGCTGGTC |
| | | CTGGAAAAAGAAAAGCTGAGCGCCATGCAGGCCCACCTGGCCGGAAAAA |
| | | TGGCCCTGACAAAGGCCAGCAGCGTGGCCTCTTCTGATAAGGGCAGCTGC |
| | | TGCATTGTGGCCGCTGGATCTCAGGGACCTGTGGTTCCTGCTTGGAGCGG |
| | | ACCTAGAGAGGCCCCTGATTCTCTGTTTGCCGTGCGGAGACACCTGTGGG |
| | | GCTCTCACGGCAACTCTACTTTCCCCGAGTTCCTGCACAACATGGACTACT |
| | | TCAAGTTCCACAACATGCGGCCTCCATTCACCTACGCCACACTGATCAGA |
| | | TGGGCCATTCTGGAAGCCCCTGAGAAGCAGAGAACCCTGAACGAGATCT |
| | | ACCACTGGTTTACCCGGATGTTCGCCTTCTTCCGGAATCACCCTGCCACCT |
| | | GGAAGAACGCCATCCGGCACAATCTGAGCCTGCACAAGTGCTTCGTGCGC |
| | | GTGGAATCTGAGAAAGGCGCCGTGTGGACAGTGGACGAGCTGGAATTCA |
| | | GAAAGAAGAGAAGCCAGCGGCCTAGCCGGTGCAGCAATCCTACACCTGG |
| | | ACCTGGAAGCGGAGCGACTAACTTCAGCCTGCTGAAGCAGGCCGGAGAT |
| | | GTGGAGGAAAACCCTGGACCGATGGGGGCAGGTGCCACCGGACGAGCCA |
| | | TGGACGGGCCGCGCCTGCTGCTGTTGCTGCTTCTGGGGGTGTCCCTTGGA |
| | | GGTGCCAAGGAGGCATGCCCCACAGGCCTGTACACACACAGCGGTGAGT |
| | | GCTGCAAAGCCTGCAACCTGGGCGAGGGTGTGGCCCAGCCTTGTGGAGC |
| | | CAACCAGACCGTGTGTGAGCCCTGCCTGGACAGCGTGACGTTCTCCGACG |
| | | TGGTGAGCGCGACCGAGCCGTGCAAGCCGTGCACCGAGTGCGTGGGGCT |
| | | CCAGAGCATGTCGGCGCCGTGCGTGGAGGCCGACGACGCCGTGTGCCGC |
| | | TGCGCCTACGGCTACTACCAGGATGAGACGACTGGGCGCTGCGAGGCGT |
| | | GCCGCGTGTGCGAGGCGGGCTCGGGCCTCGTGTTCTCCTGCCAGGACAAG |
| | | CAGAACACCGTGTGCGAGGAGTGCCCCGACGGCACGTATTCCGACGAGG |
| | | CCAACCACGTGGACCCGTGCCTGCCCTGCACCGTGTGCGAGGACACCGAG |
| | | CGCCAGCTCCGCGAGTGCACACGCTGGGCCGACGCCGAGTGCGAGGAGA |
| | | TCCCTGGCCGTTGGATTACACGGTCCACACCCCCAGAGGGCTCGGACAGC |
| | | ACAGCCCCCAGCACCCAGGAGCCTGAGGCACCTCCAGAACAAGACCTCA |
| | | TAGCCAGCACGGTGGCAGGTGTGGTGACCACAGTGATGGGCAGCTCCCA |
| | | GCCCGTGGTGACCCGAGGCACCACCGACAACCTCATCCCTGTCTATTGCT |
| | | CCATCCTGGCTGCTGTGGTTGTGGGTCTTGTGGCCTACATAGCCTTCAAGA |
| | | GGTGA |
| 38 | LNGFR-P2A- | GCCACCATGGGGGCAGGTGCCACCGGACGAGCCATGGACGGGCCGCGCC |
| | FOXP3cDNA | TGCTGCTGTTGCTGCTTCTGGGGGTGTCCCTTGGAGGTGCCAAGGAGGCA |
| | | TGCCCCACAGGCCTGTACACACACAGCGGTGAGTGCTGCAAAGCCTGCA |
| | | ACCTGGGCGAGGGTGTGGCCCAGCCTTGTGGAGCCAACCAGACCGTGTGT |
| | | GAGCCCTGCCTGGACAGCGTGACGTTCTCCGACGTGGTGAGCGCGACCGA |
| | | GCCGTGCAAGCCGTGCACCGAGTGCGTGGGGCTCCAGAGCATGTCGGCG |
| | | CCGTGCGTGGAGGCCGACGACGCCGTGTGCCGCTGCGCCTACGGCTACTA |
| | | CCAGGATGAGACGACTGGGCGCTGCGAGGCGTGCCGCGTGTGCGAGGCG |
| | | GGCTCGGGCCTCGTGTTCTCCTGCCAGGACAAGCAGAACACCGTGTGCGA |
| | | GGAGTGCCCCGACGGCACGTATTCCGACGAGGCCAACCACGTGGACCCG |
| | | TGCCTGCCCTGCACCGTGTGCGAGGACACCGAGCGCCAGCTCCGCGAGTG |
| | | CACACGCTGGGCCGACGCCGAGTGCGAGGAGATCCCTGGCCGTTGGATT |
| | | ACACGGTCCACACCCCCAGAGGGCTCGGACAGCACAGCCCCCAGCCCC |
| | | AGGAGCCTGAGGCACCTCCAGAACAAGACCTCATAGCCAGCACGGTGGC |
| | | AGGTGTGGTGACCACAGTGATGGGCAGCTCCCAGCCCGTGGTGACCCGA |
| | | GGCACCACCGACAACCTCATCCCTGTTATTGCTCCATCCTGGCTGCTGTG |
| | | GTTGTGGGTCTTGTGGCCTACATAGCCTTCAAGAGGGGAAGCGGAGCGAC |
| | | TAACTTCAGCCTGCTGAAGCAGGCCGGAGATGTGGAGGAAAACCCTGGA |
| | | CCGATGCCTAATCCTCGGCCTGGAAAGCCTAGCGCTCCTTCTCTTGCTCTG |
| | | GGACCTTCTCCTGGCGCCTCTCCATCTTGGAGAGCCGCTCCTAAAGCCAG |
| | | CGATCTGCTGGGAGCTAGAGGACCTGGCGGCACATTTCAGGGCAGAGAT |
| | | CTTAGAGGCGGAGCCCACGCTAGCTCCTCCAGCCTTAATCCTATGCCTCC |
| | | TAGCCAGCTCCAGCTGCCTACACTGCCTCTGGTTATGGTGGCTCCTAGCG |
| | | GAGCTAGACTGGGCCCTCTGCCTCATCTGCAAGCTCTGCTGCAGGACAGA |
| | | CCCCACTTCATGCACCAGCTGAGCACCGTGGATGCCCACGCAAGAACACC |
| | | TGTGCTGCAGGTTCACCCTCTGGAATCCCCAGCCATGATCAGCCTGACAC |
| | | CTCCAACAACAGCCACCGGCGTGTTCAGCCTGAAAGCCAGACCTGGACTG |
| | | CCTCCTGGCATCAATGTGGCCAGCCTGGAATGGGTGTCCAGAGAACCTGC |
| | | TCTGCTGTGCACATTCCCCAATCCAAGCGCTCCCAGAAAGGACAGCACAC |
| | | TGTCTGCCGTGCCTCAGAGCAGCTATCCCCTGCCTTGCTAACGGCGTGTGC |
| | | AAGTGGCCTGGATGCGAGAAGGTGTTCGAGGAACCCGAGGACTTCCTGA |
| | | AGCACTGCCAGGCCGATCATCTGCTGGACGAGAAAGGCAGAGCCCAGTG |
| | | TCTGCTCCAGCGCGAGATGGTGCAGTCTCTGGAACAGCAGCTGGTCCTGG |
| | | AAAAAGAAAAGCTGAGCGCCATGCAGGCCCACCTGGCCGGAAAAATGGC |
| | | CCTGACAAAGGCCAGCAGCGTGGCCTCTTCTGATAAGGGCAGCTGCTGCA |
| | | TTGTGGCCGCTGGATCTCAGGGACCTGTGGTTCCTGCTTGGAGCGGACCT |
| | | AGAGAGGCCCCTGATTCTCTGTTTGCCGTGCGGAGACACCTGTGGGGCTC |
| | | TCACGGCAACTCTACTTTCCCCGAGTTCCTGCACAACATGGACTACTTCA |
| | | AGTTCCACAACATGCGGCCTCCATTCACCTACGCCACACTGATCAGATGG |
| | | GCCATTCTGGAAGCCCCTGAGAAGCAGAGAACCCTGAACGAGATCTACC |
| | | ACTGGTTTACCCGGATGTTCGCCTTCCTTCCGGAATCACCCTGCCACCTGGA |
| | | AGAACGCCATCCGGCACAATCTGAGCCTGCACAAGTGCTTCGTGCGCGTG |
| | | GAATCTGAGAAAGGCGCCGTGTGGACAGTGGACGAGCTGGAATTCAGAA |
| | | AGAAGAGAAGCCAGCGGCCTAGCCGGTGGAGCAATCCTACACCTGGACC |
| | | TTGA |
| 39 | FOXP3cDNA- | ATGCCTAATCCTCGGCCTGGAAAGCCTAGCGCTCCTTCTCTTGCTCTGGGA |
| | μDISC | CCTTCTCCTGGCGCCTCTCCATCTTGGAGAGCCGCTCCTAAAGCCAGCGA |
| | nucleotide | TCTGCTGGGAGCTAGAGGACCTGGCGGCACATTTCAGGGCAGAGATCTTA |
| | sequence | GAGGCGGAGCCCACGCTAGCTCCTCCAGCCTTAATCCTATGCCTCCTAGC |
| | | CAGCTCCAGCTGCCTACACTGCCTCTGGTTATGGTGGCTCCTAGCGGAGC |
| | | TAGACTGGGCCCTCTGCCTCATCTGCAAGCTCTGCTGCAGGACAGACCCC |
| | | ACTTCATGCACCAGCTGAGCACCGTGGATGCCCACGCAAGAACACCTGTG |
| | | CTGCAGGTTCACCCTCTGGAATCCCCAGCCATGATCAGCCTGACACCTCC |
| | | AACAACAGCCACCGGCGTGTTCAGCCTGAAAGCCAGACCTGGACTGCCTC |
| | | CTGGCATCAATGTGGCCAGCCTGGAATGGGTGTCCAGAGAACCTGCTCTG |
| | | CTGTGCACATTCCCCAATCCAAGCGCTCCCAGAAAGGACAGCACACTGTC |
| | | TGCCGTGCCTCAGAGCAGCTATCCCCTGCTTGCTAACGGCGTGTGCAAGT |
| | | GGCCTGGATGCGAGAAGGTGTTCGAGGAACCCGAGGACTTCCTGAAGCA |
| | | CTGCCAGGCCGATCATCTGCTGGACGAGAAAGGCAGAGCCCAGTGTCTG |
| | | CTCCAGCGCGAGATGGTGCAGTCTCTGGAACAGCAGCTGGTCCTGGAAA |
| | | AAGAAAAGCTGAGCGCCATGCAGGCCCACCTGGCCGGAAAAATGGCCCT |
| | | GACAAAGGCCAGCAGCGTGGCCTCTTCTGATAAGGGCAGCTGCTGCATTG |
| | | TGGCCGCTGGATCTCAGGGACCTGTGGTTCCTGCTTGGAGCGGACCTAGA |
| | | GAGGCCCCTGATTCTCTGTTTGCCGTGCGGAGACACCTGTGGGGCTCTCA |
| | | CGGCAACTCTACTTTCCCCGAGTTCCTGCACAACATGGACTACTTCAAGTT |
| | | CCACAACATGCGGCCTCCATTCACCTACGCCACACTGATCAGATGGGCCA |
| | | TTCTGGAAGCCCCTGAGAAGCAGAGAACCCTGAACGAGATCTACCACTG |
| | | GTTTACCCGGATGTTCGCCTTCTTCCGGAATCACCCTGCCACCTGGAAGA |
| | | ACGCCATCCGGCACAATCTGAGCCTGCACAAGTGCTTCGTGCGCGTGGAA |
| | | TCTGAGAAAGGCGCCGTGTGGACAGTGGACGAGCTGGAATTCAGAAAGA |
| | | AGAGAAGCCAGCGGCCTAGCCGGTGCAGCAATCCTACACCTGGACCTGG |
| | | AAGCGGAGCGACTAACTTCAGCCTGCTTAAGCAGGCCGGAGATGTGGAG |
| | | GAAAACCCTGGACCGATGCCTCTGGGCCTGCTGTGGCTGGGCCTGGCCCT |
| | | GCTGGGCGCCCTGCACGCCCAGGCCGGCGTGCAGGTGGAGACAATCTCC |
| | | CCAGGCGACGGACGCACATTCCCTAAGCGGGGCCAGACCTGCGTGGTGC |
| | | ACTATACAGGCATGCTGGAGGATGGCAAGAAGTTTGACAGCTCCCGGGA |
| | | TAGAAACAAGCCATTCAAGTTTATGCTGGGCAAGCAGGAAGTGATCAGA |
| | | GGCTGGGAGGAGGGCGTGGCCCAGATGTCTGTGGGCCAGAGGGCCAAGC |
| | | TGACCATCAGCCCAGACTACGCCTATGGAGCAACAGGCCACCCAGGAAT |
| | | CATCCCACCTCACGCCACCCTGGTGTTCGATGTGGAGCTGCTGAAGCTGG |
| | | GCGAGGGAGGGTCACCTGGATCCAACACATCAAAAGAGAACCCCTTTCT |
| | | GTTCGCATTGGAGGCCGTAGTCATATCTGTTGGATCCATGGGACTTATTAT |
| | | CTCCCTGTTGTGTGTGTACTTCTGGCTGGAACGGACTATGCCCAGGATCCC |
| | | CACGCTCAAGAATCTGGAAGATCTCGTCACAGAATACCATGGTAATTTCA |
| | | GCGCCTGGAGCGGAGTCTCTAAGGGTCTGGCCGAATCCCTCCAACCCGAT |
| | | TATTCTGAACGGTTGTGCCTCGTATCCGAAATACCACCAAAAGGCGGGGC |
| | | TCTGGGTGAGGGCCCAGGGGCGAGTCCGTGCAATCAACACAGCCCGTATT |
| | | GGGCCCCTCCTTGTTATACGTTGAAGCCCGAAACTGGAAGCGGAGCTACT |
| | | AACTTCAGCCTGCTGAAGCAGGCTGGAGACGTGGAGGAGAACCCTGGAC |
| | | CTATGGCACTGCCCGTGACCGCCCTGCTGCTGCCTCTGGCCCTGCTGCTGC |
| | | ACGCAGCCCGGCCTATCCTGTGGCACGAGATGTGGCACGAGGGCCTGGA |
| | | GGAGGCCAGCAGGCTGTATTTTGGCGAGCGCAACGTGAAGGGCATGTTC |
| | | GAGGTGCTGGAGCCTCTGCACGCCATGATGGAGAGAGGCCCACAGACCC |
| | | TGAAGGAGACATCCTTTAACCAGGCCTATGGACGGGACCTGATGGAGGC |
| | | ACAGGAGTGGTGCAGAAAGTACATGAAGTCTGGCAATGTGAAGGACCTG |
| | | CTGCAGGCCTGGGATCTGTACTATCACGTGTTTCGGAGAATCTCCAAGCC |
| | | AGCAGCTCTCGGCAAAGACACGATTCCGTGGCTTGGGCATCTGCTCGTTG |
| | | GGCTGAGCGGTGCGTTTGGTTTCATCATCTTGGTCTATCTCTTGATCAATT |
| | | GCAGAAATACAGGCCCTTGGCTGAAAAAAGTGCTCAAGTGTAATACCCC |
| | | CGACCCAAGCAAGTTCTTCTCCCAGCTTTCTTCAGAGCATGGAGGCGATG |
| | | TGCAGAAATGGCTCTCTTCACCTTTTCCCTCCTCAAGCTTCTCCCCGGGAG |
| | | GGCTGGCGCCCGAGATTTCACCTCTTGAGGTACTTGAACGAGACAAGGTT |
| | | ACCCAACTTCTCCTTCAACAGGATAAGGTACCCGAACCTGCGAGCCTTAG |
| | | CTTGAATACAGACGCTTATCTCTCACTGCAGGAACTGCAAGGATCTGGTG |
| | | CTACTAATTTTTCTCTTTTGAAGCAAGCTGGAGATGTTGAAGAGAACCCC |
| | | GGTCCGGAGATGTGGCATGAGGGTCTGGAAGAAGCGTCTCGACTGTACTT |
| | | TGGTGAGCGCAATGTGAAGGGCATGTTTGAAGTCCTCGAACCCCTTCATG |
| | | CCATGATGGAACGCGGACCCCAGACCTTGAAGGAGACAAGTTTTAACCA |
| | | AGCTTACGGAAGAGACCTGATGGAAGCCCAGGAATGGTGCAGGAAATAC |
| | | ATGAAAAGCGGGAATGTGAAGGACTTGCTCCAAGCGTGGGACCTGTACT |
| | | ATCATGTCTTTAGGCGCATTAGTAAGTGA |
| 40 | FOXP3cDNA- | ATGCCTAATCCTCGGCCTGGAAAGCCTAGCGCTCCTTCTCTTGCTCTGGGA |
| | LNGFRe-μDISC | CCTTCTCCTGGCGCCTCTCCATCTTGGAGAGCCGCTCCTAAAGCCAGCGA |
| | nucleotide | TCTGCTGGGAGCTAGAGGACCTGGCGGCACATTTCAGGGCAGAGATCTTA |
| | sequence | GAGGCGGAGCCCACGCTAGCTCCTCCAGCCTTAATCCTATGCCTCCTAGC |
| | | CAGCTCCAGCTGCCTACACTGCCTCTGGTTATGGTGGCTCCTAGCGGAGC |
| | | TAGACTGGGCCCTCTGCCTCATCTGCAAGCTCTGCTGCAGGACAGACCCC |
| | | ACTTCATGCACCAGCTGAGCACCGTGGATGCCCACGCAAGAACACCTGTG |
| | | CTGCAGGTTCACCCTCTGGAATCCCCAGCCATGATCAGCCTGACACCTCC |
| | | AACAACAGCCACCGGCGTGTTCAGCCTGAAAGCCAGACCTGGACTGCCTC |
| | | CTGGCATCAATGTGGCCAGCCTGGAATGGGTGTCCAGAGAACCTGCTCTG |
| | | CTGTGCACATTCCCCAATCCAAGCGCTCCCAGAAAGGACAGCACACTGTC |
| | | TGCCGTGCCTCAGAGCAGCTATCCCCTGCTTGCTAACGGCGTGTGCAAGT |
| | | GGCCTGGATGCGAGAAGGTGTTCGAGGAACCCGAGGACTTCCTGAAGCA |
| | | CTGCCAGGCCGATCATCTGCTGGACGAGAAAGGCAGAGCCCAGTGTCTG |
| | | CTCCAGCGCGAGATGGTGCAGTCTCTGGAACAGCAGCTGGTCCTGGAAA |
| | | AAGAAAAGCTGAGCGCCATGCAGGCCCACCTGGCCGGAAAAATGGCCCT |
| | | GACAAAGGCCAGCAGCGTGGCCTCTTCTGATAAGGGCAGCTGCTGCATTG |
| | | TGGCCGCTGGATCTCAGGGACCTGTGGTTCCTGCTTGGAGCGGACCTAGA |
| | | GAGGCCCCTGATTCTCTGTTTGCCGTGCGGAGACACCTGTGGGGCTCTCA |
| | | CGGCAACTCTACTTTCCCCGAGTTCCTGCACAACATGGACTACTTCAAGTT |
| | | CCACAACATGCGGCCTCCATTCACCTACGCCACACTGATCAGATGGGCCA |
| | | TTCTGGAAGCCCCTGAGAAGCAGAGAACCCTGAACGAGATCTACCACTG |
| | | GTTTACCCGGATGTTCGCCTTCTTCCGGAATCACCCTGCCACCTGGAAGA |
| | | ACGCCATCCGGCACAATCTGAGCCTGCACAAGTGCTTCGTGCGCGTGGAA |
| | | TCTGAGAAAGGCGCCGTGTGGACAGTGGACGAGCTGGAATTCAGAAAGA |
| | | AGAGAAGCCAGCGGCCTAGCCGGTGCAGCAATCCTACACCTGGACCTGG |
| | | AAGCGGAGCGACTAACTTCAGCCTGCTTAAGCAGGCCGGAGATGTGGAG |
| | | GAAAACCCTGGACCGATGCCTCTGGGCCTGCTGTGGCTGGGCCTGGCCCT |
| | | GCTGGGCGCCCTGCACGCCCAGGCCATGGGGGCAGGTGCCACCGGACGA |
| | | GCCATGGACGGGCCGCGCCTGCTGCTGTTGCTGCTTCTGGGGGTGTCCCT |
| | | TGGAGGTGCCAAGGAGGCATGCCCCACAGGCCTGTACACACACAGCGGT |
| | | GAGTGCTGCAAAGCCTGCAACCTGGGCGAGGGTGTGGCCCAGCCTTGTG |
| | | GAGCCAACCAGACCGTGTGTGAGCCCTGCCTGGACAGCGTGACGTTCTCC |
| | | GACGTGGTGAGCGCGACCGAGCCGTGCAAGCCGTGCACCGAGTGCGTGG |
| | | GGCTCCAGAGCATGTCGGCGCCGTGCGTGGAGGCCGACGACGCCGTGTG |
| | | CCGCTGCGCCTACGGCTACTACCAGGATGAGACGACTGGGCGCTGCGAG |
| | | GCGTGCCGCGTGTGCGAGGCGGGCTCGGGCCTCGTGTTCTCCTGCCAGGA |
| | | CAAGCAGAACACCGTGTGCGAGGAGTGCCCCGACGGCACGTATTCCGAC |
| | | GAGGCCAACCACGTGGACCCGTGCCTGCCCTGCACCGTGTGCGAGGACA |
| | | CCGAGCGCCAGCTCCGCGAGTGCACACGCTGGGCCGACGCCGAGTGCGA |
| | | GGAGATCCCTGGCCGTTGGATTACACGGTCCACACCCCCAGAGGGCTCGG |
| | | ACAGCACAGCCCCCAGCACCCAGGAGCCTGAGGCACCTCCAGAACAAGA |
| | | CCTCATAGCCAGCACGGTGGCAGGTGTGGTGACCACAGTGATGGGCAGC |
| | | TCCCAGCCCGTGGTGACCCGAGGCACCACCGACAACCTCATCCCTGTCTA |
| | | TTGCTCCATCCTGGCTGCTGTGGTTGTGGGTCTTGTGGCCTACATAGCCTT |
| | | CAAGAGGGGCGTGCAGGTGGAGACAATCTCCCCAGGCGACGGACGCACA |
| | | TTCCCTAAGCGGGGCCAGACCTGCGTGGTGCACTATACAGGCATGCTGGA |
| | | GGATGGCAAGAAGTTTGACAGCTCCCGGGATAGAAACAAGCCATTCAAG |
| | | TTTATGCTGGGCAAGCAGGAAGTGATCAGAGGCTGGGAGGAGGGCGTGG |
| | | CCCAGATGTCTGTGGGCCAGAGGGCCAAGCTGACCATCAGCCCAGACTA |
| | | CGCCTATGGAGCAACAGGCCACCCAGGAATCATCCCACCTCACGCCACCC |
| | | TGGTGTTCGATGTGGAGCTGCTGAAGCTGGGCGAGGGAGGGTCACCTGG |
| | | ATCCAACACATCAAAAGAGAACCCCTTTCTGTTCGCATTGGAGGCCGTAG |
| | | TCATATCTGTTGGATCCATGGGACTTATTATCTCCCTGTTGTGTGTGTACT |
| | | TCTGGCTGGAACGGACTATGCCCAGGATCCCCACGCTCAAGAATCTGGAA |
| | | GATCTCGTCACAGAATACCATGGTAATTTCAGCGCCTGGAGCGGAGTCTC |
| | | TAAGGGTCTGGCCGAATCCCTCCAACCCGATTATTCTGAACGGTTGTGCC |
| | | TCGTATCCGAAATACCACCAAAAGGCGGGGCTCTGGGTGAGGGCCCAGG |
| | | GGCGAGTCCGTGCAATCAACACAGCCCGTATTGGGCCCCTCCTTGTTATA |
| | | CGTTGAAGCCCGAAACTGGAAGCGGAGCTACTAACTTCAGCCTGCTGAA |
| | | GCAGGCTGGAGACGTGGAGGAGAACCCTGGACCTATGGCACTGCCCGTG |
| | | ACCGCCCTGCTGCTGCCTCTGGCCCTGCTGCTGCACGCAGCCCGGCCTAT |
| | | CCTGTGGCACGAGATGTGGCACGAGGGCCTGGAGGAGGCCAGCAGGCTG |
| | | TATTTTGGCGAGCGCAACGTGAAGGGCATGTTCGAGGTGCTGGAGCCTCT |
| | | GCACGCCATGATGGAGAGAGGCCCACAGACCCTGAAGGAGACATCCTTT |
| | | AACCAGGCCTATGGACGGGACCTGATGGAGGCACAGGAGTGGTGCAGAA |
| | | AGTACATGAAGTCTGGCAATGTGAAGGACCTGCTGCAGGCCTGGGATCTG |
| | | TACTATCACGTGTTTCGGAGAATCTCCAAGCCAGCAGCTCTCGGCAAAGA |
| | | CACGATTCCGTGGCTTGGGCATCTGCTCGTTGGGCTGAGCGGTGCGTTTG |
| | | GTTTCATCATCTTGGTCTATCTCTTGATCAATTGCAGAAATACAGGCCCTT |
| | | GGCTGAAAAAAGTGCTCAAGTGTAATACCCCCGACCCAAGCAAGTTCTTC |
| | | TCCCAGCTTTCTTCAGAGCATGGAGGCGATGTGCAGAAATGGCTCTCTTC |
| | | ACCTTTTCCCTCCTCAAGCTTCTCCCCGGGAGGGCTGGCGCCCGAGATTTC |
| | | ACCTCTTGAGGTACTTGAACGAGACAAGGTTACCCAACTTCTCCTTCAAC |
| | | AGGATAAGGTACCCGAACCTGCGAGCCTTAGCTTGAATACAGACGCTTAT |
| | | CTCTCACTGCAGGAACTGCAAGGATCTGGTGCTACTAATTTTTCTCTTTTG |
| | | AAGCAAGCTGGAGATGTTGAAGAGAACCCCGGTCCGGAGATGTGGCATG |
| | | AGGGTCTGGAAGAAGCGTCTCGACTGTACTTTGGTGAGCGCAATGTGAAG |
| | | GGCATGTTTGAAGTCCTCGAACCCCTTCATGCCATGATGGAACGCGGACC |
| | | CCAGACCTTGAAGGAGACAAGTTTTAACCAAGCTTACGGAAGAGACCTG |
| | | ATGGAAGCCCAGGAATGGTGCAGGAAATACATGAAAAGCGGGAATGTGA |
| | | AGGACTTGCTCCAAGCGTGGGACCTGTACTATCATGTCTTTAGGCGCATT |
| | | AGTAAG |
| 41 | μDISC- | ATGCCTCTGGGCCTGCTGTGGCTGGGCCTGGCCCTGCTGGGCGCCCTGCA |
| | FOXP3cDNA | CGCCCAGGCCGGCGTGCAGGTGGAGACAATCTCCCCAGGCGACGGACGC |
| | nucleotide | ACATTCCCTAAGCGGGGCCAGACCTGCGTGGTGCACTATACAGGCATGCT |
| | sequence | GGAGGATGGCAAGAAGTTTGACAGCTCCCGGGATAGAAACAAGCCATTC |
| | | AAGTTTATGCTGGGCAAGCAGGAAGTGATCAGAGGCTGGGAGGAGGGCG |
| | | TGGCCCAGATGTCTGTGGGCCAGAGGGCCAAGCTGACCATCAGCCCAGA |
| | | CTACGCCTATGGAGCAACAGGCCACCCAGGAATCATCCCACCTCACGCCA |
| | | CCCTGGTGTTCGATGTGGAGCTGCTGAAGCTGGGCGAGGGAGGGTCACCT |
| | | GGATCCAACACATCAAAAGAGAACCCCTTTCTGTTCGCATTGGAGGCCGT |
| | | AGTCATATCTGTTGGATCCATGGGACTTATTATCTCCCTGTTGTGTGTGTA |
| | | CTTCTGGCTGGAACGGACTATGCCCAGGATCCCCACGCTCAAGAATCTGG |
| | | AAGATCTCGTCACAGAATACCATGGTAATTTCAGCGCCTGGAGCGGAGTC |
| | | TCTAAGGGTCTGGCCGAATCCCTCCAACCCGATTATTCTGAACGGTTGTG |
| | | CCTCGTATCCGAAATACCACCAAAAGGCGGGGCTCTGGGTGAGGGCCCA |
| | | GGGGCGAGTCCGTGCAATCAACACAGCCCGTATTGGGCCCCTCCTTGTTA |
| | | TACGTTGAAGCCCGAAACTGGAAGCGGAGCTACTAACTTCAGCCTGCTGA |
| | | AGCAGGCTGGAGACGTGGAGGAGAACCCTGGACCTATGGCACTGCCCGT |
| | | GACCGCCCTGCTGCTGCCTCTGGCCCTGCTGCTGCACGCAGCCCGGCCTA |
| | | TCCTGTGGCACGAGATGTGGCACGAGGGCCTGGAGGAGGCCAGCAGGCT |
| | | GTATTTTGGCGAGCGCAACGTGAAGGGCATGTTCGAGGTGCTGGAGCCTC |
| | | TGCACGCCATGATGGAGAGAGGCCCACAGACCCTGAAGGAGACATCCTT |
| | | TAACCAGGCCTATGGACGGGACCTGATGGAGGCACAGGAGTGGTGCAGA |
| | | AAGTACATGAAGTCTGGCAATGTGAAGGACCTGCTGCAGGCCTGGGATCT |
| | | GTACTATCACGTGTTTCGGAGAATCTCCAAGCCAGCAGCTCTCGGCAAAG |
| | | ACACGATTCCGTGGCTTGGGCATCTGCTCGTTGGGCTGAGCGGTGCGTTT |
| | | GGTTTCATCATCTTGGTCTATCTCTTGATCAATTGCAGAAATACAGGCCCT |
| | | TGGCTGAAAAAAGTGCTCAAGTGTAATACCCCCGACCCAAGCAAGTTCTT |
| | | CTCCCAGCTTTCTTCAGAGCATGGAGGCGATGTGCAGAAATGGCTCTCTT |
| | | CACCTTTTCCCTCCTCAAGCTTCTCCCCGGGAGGGCTGGCGCCCGAGATTT |
| | | CACCTCTTGAGGTACTTGAACGAGACAAGGTTACCCAACTTCTCCTTCAA |
| | | CAGGATAAGGTACCCGAACCTGCGAGCCTTAGCTTGAATACAGACGCTTA |
| | | TCTCTCACTGCAGGAACTGCAAGGATCTGGTGCTACTAATTTTTCTCTTTT |
| | | GAAGCAAGCTGGAGATGTTGAAGAGAACCCCGGTCCGGAGATGTGGCAT |
| | | GAGGGTCTGGAAGAAGCGTCTCGACTGTACTTTGGTGAGCGCAATGTGAA |
| | | GGGCATGTTTGAAGTCCTCGAACCCCTTCATGCCATGATGGAACGCGGAC |
| | | CCCAGACCTTGAAGGAGACAAGTTTTAACCAAGCTTACGGAAGAGACCT |
| | | GATGGAAGCCCAGGAATGGTGCAGGAAATACATGAAAAGCGGGAATGTG |
| | | AAGGACTTGCTCCAAGCGTGGGACCTGTACTATCATGTCTTTAGGCGCAT |
| | | TAGTAAGGGAAGCGGAGCGACTAACTTCAGCCTGCTTAAGCAGGCCGGA |
| | | GATGTGGAGGAAAACCCTGGACCGATGCCTAATCCTCGGCCTGGAAAGC |
| | | CTAGCGCTCCTTCTCTTGCTCTGGGACCTTCTCCTGGCGCCTCTCCATCTT |
| | | GGAGAGCCGCTCCTAAAGCCAGCGATCTGCTGGGAGCTAGAGGACCTGG |
| | | CGGCACATTTCAGGGCAGAGATCTTAGAGGCGGAGCCCACGCTAGCTCCT |
| | | CCAGCCTTAATCCTATGCCTCCTAGCCAGCTCCAGCTGCCTACACTGCCTC |
| | | TGGTTATGGTGGCTCCTAGCGGAGCTAGACTGGGCCCTCTGCCTCATCTG |
| | | CAAGCTCTGCTGCAGGACAGACCCCACTTCATGCACCAGCTGAGCACCGT |
| | | GGATGCCCACGCAAGAACACCTGTGCTGCAGGTTCACCCTCTGGAATCCC |
| | | CAGCCATGATCAGCCTGACACCTCCAACAACAGCCACCGGCGTGTTCAGC |
| | | CTGAAAGCCAGACCTGGACTGCCTCCTGGCATCAATGTGGCCAGCCTGGA |
| | | ATGGGTGTCCAGAGAACCTGCTCTGCTGTGCACATTCCCCAATCCAAGCG |
| | | CTCCCAGAAAGGACAGCACACTGTCTGCCGTGCCTCAGAGCAGCTATCCC |
| | | CTGCTTGCTAACGGCGTGTGCAAGTGGCCTGGATGCGAGAAGGTGTTCGA |
| | | GGAACCCGAGGACTTCCTGAAGCACTGCCAGGCCGATCATCTGCTGGACG |
| | | AGAAAGGCAGAGCCCAGTGTCTGCTCCAGCGCGAGATGGTGCAGTCTCT |
| | | GGAACAGCAGCTGGTCCTGGAAAAAGAAAAGCTGAGCGCCATGCAGGCC |
| | | CACCTGGCCGGAAAAATGGCCCTGACAAAGGCCAGCAGCGTGGCCTCTT |
| | | CTGATAAGGGCAGCTGCTGCATTGTGGCCGCTGGATCTCAGGGACCTGTG |
| | | GTTCCTGCTTGGAGCGGACCTAGAGAGGCCCCTGATTCTCTGTTTGCCGT |
| | | GCGGAGACACCTGTGGGGCTCTCACGGCAACTCTACTTTCCCCGAGTTCC |
| | | TGCACAACATGGACTACTTCAAGTTCCACAACATGCGGCCTCCATTCACC |
| | | TACGCCACACTGATCAGATGGGCCATTCTGGAAGCCCCTGAGAAGCAGA |
| | | GAACCCTGAACGAGATCTACCACTGGTTTACCCGGATGTTCGCCTTCTTCC |
| | | GGAATCACCCTGCCACCTGGAAGAACGCCATCCGGCACAATCTGAGCCTG |
| | | CACAAGTGCTTCGTGCGCGTGGAATCTGAGAAAGGCGCCGTGTGGACAG |
| | | TGGACGAGCTGGAATTCAGAAAGAAGAGAAGCCAGCGGCCTAGCCGGTG |
| | | CAGCAATCCTACACCTGGACCT |
| 42 | LNGFRe-μDISC | ATGCCTCTGGGCCTGCTGTGGCTGGGCCTGGCCCTGCTGGGCGCCCTGCA |
| | -FOXP3cDNA | CGCCCAGGCCATGGGGGCAGGTGCCACCGGACGAGCCATGGACGGGCCG |
| | nucleotide | CGCCTGCTGCTGTTGCTGCTTCTGGGGGTGTCCCTTGGAGGTGCCAAGGA |
| | sequence | GGCATGCCCCACAGGCCTGTACACACACAGCGGTGAGTGCTGCAAAGCC |
| | | TGCAACCTGGGCGAGGGTGTGGCCCAGCCTTGTGGAGCCAACCAGACCG |
| | | TGTGTGAGCCCTGCCTGGACAGCGTGACGTTCTCCGACGTGGTGAGCGCG |
| | | ACCGAGCCGTGCAAGCCGTGCACCGAGTGCGTGGGGCTCCAGAGCATGT |
| | | CGGCGCCGTGCGTGGAGGCCGACGACGCCGTGTGCCGCTGCGCCTACGG |
| | | CTACTACCAGGATGAGACGACTGGGCGCTGCGAGGCGTGCCGCGTGTGC |
| | | GAGGCGGGCTCGGGCCTCGTGTTCTCCTGCCAGGACAAGCAGAACACCGT |
| | | GTGCGAGGAGTGCCCCGACGGCACGTATTCCGACGAGGCCAACCACGTG |
| | | GACCCGTGCCTGCCCTGCACCGTGTGCGAGGACACCGAGCGCCAGCTCCG |
| | | CGAGTGCACACGCTGGGCCGACGCCGAGTGCGAGGAGATCCCTGGCCGT |
| | | TGGATTACACGGTCCACACCCCCAGAGGGCTCGGACAGCACAGCCCCCA |
| | | GCACCCAGGAGCCTGAGGCACCTCCAGAACAAGACCTCATAGCCAGCAC |
| | | GGTGGCAGGTGTGGTGACCACAGTGATGGGCAGCTCCCAGCCCGTGGTG |
| | | ACCCGAGGCACCACCGACAACCTCATCCCTGTCTATTGCTCCATCCTGGC |
| | | TGCTGTGGTTGTGGGTCTTGTGGCCTACATAGCCTTCAAGAGGGGCGTGC |
| | | AGGTGGAGACAATCTCCCCAGGCGACGGACGCACATTCCCTAAGCGGGG |
| | | CCAGACCTGCGTGGTGCACTATACAGGCATGCTGGAGGATGGCAAGAAG |
| | | TTTGACAGCTCCCGGGATAGAAACAAGCCATTCAAGTTTATGCTGGGCAA |
| | | GCAGGAAGTGATCAGAGGCTGGGAGGAGGGCGTGGCCCAGATGTCTGTG |
| | | GGCCAGAGGGCCAAGCTGACCATCAGCCCAGACTACGCCTATGGAGCAA |
| | | CAGGCCACCCAGGAATCATCCCACCTCACGCCACCCTGGTGTTCGATGTG |
| | | GAGCTGCTGAAGCTGGGCGAGGGAGGGTCACCTGGATCCAACACATCAA |
| | | AAGAGAACCCCTTTCTGTTCGCATTGGAGGCCGTAGTCATATCTGTTGGA |
| | | TCCATGGGACTTATTATCTCCCTGTTGTGTGTGTACTTCTGGCTGGAACGG |
| | | ACTATGCCCAGGATCCCCACGCTCAAGAATCTGGAAGATCTCGTCACAGA |
| | | ATACCATGGTAATTTCAGCGCCTGGAGCGGAGTCTCTAAGGGTCTGGCCG |
| | | AATCCCTCCAACCCGATTATTCTGAACGGTTGTGCCTCGTATCCGAAATA |
| | | CCACCAAAAGGCGGGGCTCTGGGTGAGGGCCCAGGGGCGAGTCCGTGCA |
| | | ATCAACACAGCCCGTATTGGGCCCCTCCTTGTTATACGTTGAAGCCCGAA |
| | | ACTGGAAGCGGAGCTACTAACTTCAGCCTGCTGAAGCAGGCTGGAGACG |
| | | TGGAGGAGAACCCTGGACCTATGGCACTGCCCGTGACCGCCCTGCTGCTG |
| | | CCTCTGGCCCTGCTGCTGCACGCAGCCCGGCCTATCCTGTGGCACGAGAT |
| | | GTGGCACGAGGGCCTGGAGGAGGCCAGCAGGCTGTATTTTGGCGAGCGC |
| | | AACGTGAAGGGCATGTTCGAGGTGCTGGAGCCTCTGCACGCCATGATGG |
| | | AGAGAGGCCCACAGACCCTGAAGGAGACATCCTTTAACCAGGCCTATGG |
| | | ACGGGACCTGATGGAGGCACAGGAGTGGTGCAGAAAGTACATGAAGTCT |
| | | GGCAATGTGAAGGACCTGCTGCAGGCCTGGGATCTGTACTATCACGTGTT |
| | | TCGGAGAATCTCCAAGCCAGCAGCTCTCGGCAAAGACACGATTCCGTGGC |
| | | TTGGGCATCTGCTCGTTGGGCTGAGCGGTGCGTTTGGTTTCATCATCTTGG |
| | | TCTATCTCTTGATCAATTGCAGAAATACAGGCCCTTGGCTGAAAAAAGTG |
| | | CTCAAGTGTAATACCCCCGACCCAAGCAAGTTCTTCTCCCAGCTTTCTTCA |
| | | GAGCATGGAGGCGATGTGCAGAAATGGCTCTCTTCACCTTTTCCCTCCTC |
| | | AAGCTTCTCCCCGGGAGGGCTGGCGCCCGAGATTTCACCTCTTGAGGTAC |
| | | TTGAACGAGACAAGGTTACCCAACTTCTCCTTCAACAGGATAAGGTACCC |
| | | GAACCTGCGAGCCTTAGCTTGAATACAGACGCTTATCTCTCACTGCAGGA |
| | | ACTGCAAGGATCTGGTGCTACTAATTTTTCTCTTTTGAAGCAAGCTGGAG |
| | | ATGTTGAAGAGAACCCCGGTCCGGAGATGTGGCATGAGGGTCTGGAAGA |
| | | AGCGTCTCGACTGTACTTTGGTGAGCGCAATGTGAAGGGCATGTTTGAAG |
| | | TCCTCGAACCCCTTCATGCCATGATGGAACGCGGACCCCAGACCTTGAAG |
| | | GAGACAAGTTTTAACCAAGCTTACGGAAGAGACCTGATGGAAGCCCAGG |
| | | AATGGTGCAGGAAATACATGAAAAGCGGGAATGTGAAGGACTTGCTCCA |
| | | AGCGTGGGACCTGTACTATCATGTCTTTAGGCGCATTAGTAAGGGAAGCG |
| | | GAGCGACTAACTTCAGCCTGCTTAAGCAGGCCGGAGATGTGGAGGAAAA |
| | | CCCTGGACCGATGCCTAATCCTCGGCCTGGAAAGCCTAGCGCTCCTTCTC |
| | | TTGCTCTGGGACCTTCTCCTGGCGCCTCTCCATCTTGGAGAGCCGCTCCTA |
| | | AAGCCAGCGATCTGCTGGGAGCTAGAGGACCTGGCGGCACATTTCAGGG |
| | | CAGAGATCTTAGAGGCGGAGCCCACGCTAGCTCCTCCAGCCTTAATCCTA |
| | | TGCCTCCTAGCCAGCTCCAGCTGCCTACACTGCCTCTGGTTATGGTGGCTC |
| | | CTAGCGGAGCTAGACTGGGCCCTCTGCCTCATCTGCAAGCTCTGCTGCAG |
| | | GACAGACCCCACTTCATGCACCAGCTGAGCACCGTGGATGCCCACGCAA |
| | | GAACACCTGTGCTGCAGGTTCACCCTCTGGAATCCCCAGCCATGATCAGC |
| | | CTGACACCTCCAACAACAGCCACCGGCGTGTTCAGCCTGAAAGCCAGACC |
| | | TGGACTGCCTCCTGGCATCAATGTGGCCAGCCTGGAATGGGTGTCCAGAG |
| | | AACCTGCTCTGCTGTGCACATTCCCCAATCCAAGCGCTCCCAGAAAGGAC |
| | | AGCACACTGTCTGCCGTGCCTCAGAGCAGCTATCCCCTGCTTGCTAACGG |
| | | CGTGTGCAAGTGGCCTGGATGCGAGAAGGTGTTCGAGGAACCCGAGGAC |
| | | TTCCTGAAGCACTGCCAGGCCGATCATCTGCTGGACGAGAAAGGCAGAG |
| | | CCCAGTGTCTGCTCCAGCGCGAGATGGTGCAGTCTCTGGAACAGCAGCTG |
| | | GTCCTGGAAAAAGAAAAGCTGAGCGCCATGCAGGCCCACCTGGCCGGAA |
| | | AAATGGCCCTGACAAAGGCCAGCAGCGTGGCCTCTTCTGATAAGGGCAG |
| | | CTGCTGCATTGTGGCCGCTGGATCTCAGGGACCTGTGGTTCCTGCTTGGA |
| | | GCGGACCTAGAGAGGCCCCTGATTCTCTGTTTGCCGTGCGGAGACACCTG |
| | | TGGGGCTCTCACGGCAACTCTACTTTCCCCGAGTTCCTGCACAACATGGA |
| | | CTACTTCAAGTTCCACAACATGCGGCCTCCATTCACCTACGCCACACTGA |
| | | TCAGATGGGCCATTCTGGAAGCCCCTGAGAAGCAGAGAACCCTGAACGA |
| | | GATCTACCACTGGTTTACCCGGATGTTCGCCTTCTTCCGGAATCACCCTGC |
| | | CACCTGGAAGAACGCCATCCGGCACAATCTGAGCCTGCACAAGTGCTTCG |
| | | TGCGCGTGGAATCTGAGAAAGGCGCCGTGTGGACAGTGGACGAGCTGGA |
| | | ATTCAGAAAGAAGAGAAGCCAGCGGCCTAGCCGGTGCAGCAATCCTACA |
| | | CCTGGACCTTGA |
| 43 | DISC nucleotide | ATGCCTCTGGGCCTGCTGTGGCTGGGCCTGGCCCTGCTGGGCGCCCTGCA |
| | sequence | CGCCCAGGCCGGCGTGCAGGTGGAGACAATCTCCCCAGGCGACGGACGC |
| | | ACATTCCCTAAGCGGGGCCAGACCTGCGTGGTGCACTATACAGGCATGCT |
| | | GGAGGATGGCAAGAAGTTTGACAGCTCCCGGGATAGAAACAAGCCATTC |
| | | AAGTTTATGCTGGGCAAGCAGGAAGTGATCAGAGGCTGGGAGGAGGGCG |
| | | TGGCCCAGATGTCTGTGGGCCAGAGGGCCAAGCTGACCATCAGCCCAGA |
| | | CTACGCCTATGGAGCAACAGGCCACCCAGGAATCATCCCACCTCACGCCA |
| | | CCCTGGTGTTCGATGTGGAGCTGCTGAAGCTGGGCGAGGGAGGGTCACCT |
| | | GGATCCAACACATCAAAAGAGAACCCCTTTCTGTTCGCATTGGAGGCCGT |
| | | AGTCATATCTGTTGGATCCATGGGACTTATTATCTCCCTGTTGTGTGTGTA |
| | | CTTCTGGCTGGAACGGACTATGCCCAGGATCCCCACGCTCAAGAATCTGG |
| | | AAGATCTCGTCACAGAATACCATGGTAATTTCAGCGCCTGGAGCGGAGTC |
| | | TCTAAGGGTCTGGCCGAATCCCTCCAACCCGATTATTCTGAACGGTTGTG |
| | | CCTCGTATCCGAAATACCACCAAAAGGCGGGGCTCTGGGTGAGGGCCCA |
| | | GGGGCGAGTCCGTGCAATCAACACAGCCCGTATTGGGCCCCTCCTTGTTA |
| | | TACGTTGAAGCCCGAAACTGGAAGCGGAGCTACTAACTTCAGCCTGCTGA |
| | | AGCAGGCTGGAGACGTGGAGGAGAACCCTGGACCTATGGCACTGCCCGT |
| | | GACCGCCCTGCTGCTGCCTCTGGCCCTGCTGCTGCACGCAGCCCGGCCTA |
| | | TCCTGTGGCACGAGATGTGGCACGAGGGCCTGGAGGAGGCCAGCAGGCT |
| | | GTATTTTGGCGAGCGCAACGTGAAGGGCATGTTCGAGGTGCTGGAGCCTC |
| | | TGCACGCCATGATGGAGAGAGGCCCACAGACCCTGAAGGAGACATCCTT |
| | | TAACCAGGCCTATGGACGGGACCTGATGGAGGCACAGGAGTGGTGCAGA |
| | | AAGTACATGAAGTCTGGCAATGTGAAGGACCTGCTGCAGGCCTGGGATCT |
| | | GTACTATCACGTGTTTCGGAGAATCTCCAAGCCAGCAGCTCTCGGCAAAG |
| | | ACACGATTCCGTGGCTTGGGCATCTGCTCGTTGGGCTGAGCGGTGCGTTT |
| | | GGTTTCATCATCTTGGTCTATCTCTTGATCAATTGCAGAAATACAGGCCCT |
| | | TGGCTGAAAAAAGTGCTCAAGTGTAKATCCCCCGACCCAAGCAAGTTCTT |
| | | CTCCCAGCTTTCTTCAGAGCATGGAGGCGATGTGCAGAAATGGCTCTCTT |
| | | CACCTTTTCCCTCCTCAAGCTTCTCCCCGGGAGGGCTGGCGCCCGAGATTT |
| | | CACCTCTTGAGGTACTTGAACGAGACAAGGTTACCCAACTTCTCCTTCAA |
| | | CAGGATAAGGTACCCGAACCTGCGAGCCTTAGCTCCAACCACTCTCTTAC |
| | | GAGCTGCTTCACCAATCAGGGATACTTCTTTTTCCACCTTCCCGATGCGCT |
| | | GGAAATCGAAGCTTGTCAAGTTTACTTTACCTATGATCCATATAGCGAGG |
| | | AAGATCCCGACGAAGGAGTCGCCGGTGCGCCCACGGGTTCCTCACCCCA |
| | | ACCTCTCCAGCCTCTCTCAGGAGAAGATGATGCTTATTGCACTTTTCCCAG |
| | | TAGAGACGATCTCCTCCTCTTTTCTCCATCTCTTTTGGGGGGACCTTCCCC |
| | | CCCTTCTACGGCACCTGGCGGGTCTGGTGCTGGCGAGGAGCGGATGCCGC |
| | | CGTCCCTCCAGGAGCGAGTACCACGAGATTGGGATCCCCAGCCACTTGGA |
| | | CCCCCCACCCCCGGCGTACCTGACCTTGTCGATTTTCAACCTCCCCCTGAA |
| | | TTGGTGCTGCGAGAGGCTGGGGAGGAAGTTCCGGACGCTGGGCCGAGGG |
| | | AGGGCGTGTCCTTTCCATGGAGTAGGCCTCCAGGTCAAGGCGAGTTTAGG |
| | | GCTCTCAACGCGCGGCTGCCGTTGAATACAGACGCTTATCTCTCACTGCA |
| | | GGAACTGCAAGGTCAGGACCCAACACATCTTGTAGGATCTGGTGCTACTA |
| | | ATTTTTCTCTTTTGAAGCAAGCTGGAGATGTTGAAGAGAACCCCGGTCCG |
| | | GAGATGTGGCATGAGGGTCTGGAAGAAGCGTCTCGACTGTACTTTGGTGA |
| | | GCGCAATGTGAAGGGCATGTTTGAAGTCCTCGAACCCCTTCATGCCATGA |
| | | TGGAACGCGGACCCCAGACCTTGAAGGAGACAAGTTTTAACCAAGCTTA |
| | | CGGAAGAGACCTGATGGAAGCCCAGGAATGGTGCAGGAAATACATGAAA |
| | | AGCGGGAATGTGAAGGACTTGCTCCAAGCGTGGGACCTGTACTATCATGT |
| | | CTTTAGGCGCATTAGTAAG |
| 44 | μDISC | ATGCCTCTGGGCCTGCTGTGGCTGGGCCTGGCCCTGCTGGGCGCCCTGCA |
| | nucleotide | CGCCCAGGCCGGCGTGCAGGTGGAGACAATCTCCCCAGGCGACGGACGC |
| | sequence | ACATTCCCTAAGCGGGGCCAGACCTGCGTGGTGCACTATACAGGCATGCT |
| | | GGAGGATGGCAAGAAGTTTGACAGCTCCCGGGATAGAAACAAGCCATTC |
| | | AAGTTTATGCTGGGCAAGCAGGAAGTGATCAGAGGCTGGGAGGAGGGCG |
| | | TGGCCCAGATGTCTGTGGGCCAGAGGGCCAAGCTGACCATCAGCCCAGA |
| | | CTACGCCTATGGAGCAACAGGCCACCCAGGAATCATCCCACCTCACGCCA |
| | | CCCTGGTGTTCGATGTGGAGCTGCTGAAGCTGGGCGAGGGAGGGTCACCT |
| | | GGATCCAACACATCAAAAGAGAACCCCTTTCTGTTCGCATTGGAGGCCGT |
| | | AGTCATATCTGTTGGATCCATGGGACTTATTATCTCCCTGTTGTGTGTGTA |
| | | CTTCTGGCTGGAACGGACTATGCCCAGGATCCCCACGCTCAAGAATCTGG |
| | | AAGATCTCGTCACAGAATACCATGGTAATTTCAGCGCCTGGAGCGGAGTC |
| | | TCTAAGGGTCTGGCCGAATCCCTCCAACCCGATTATTCTGAACGGTTGTG |
| | | CCTCGTATCCGAAATACCACCAAAAGGCGGGGCTCTGGGTGAGGGCCCA |
| | | GGGGCGAGTCCGTGCAATCAACACAGCCCGTATTGGGCCCCTCCTTGTTA |
| | | TACGTTGAAGCCCGAAACTGGAAGCGGAGCTACTAACTTCAGCCTGCTGA |
| | | AGCAGGCTGGAGACGTGGAGGAGAACCCTGGACCTATGGCACTGCCCGT |
| | | GACCGCCCTGCTGCTGCCTCTGGCCCTGCTGCTGCACGCAGCCCGGCCTA |
| | | TCCTGTGGCACGAGATGTGGCACGAGGGCCTGGAGGAGGCCAGCAGGCT |
| | | GTATTTTGGCGAGCGCAACGTGAAGGGCATGTTCGAGGTGCTGGAGCCTC |
| | | TGCACGCCATGATGGAGAGAGGCCCACAGACCCTGAAGGAGACATCCTT |
| | | TAACCAGGCCTATGGACGGGACCTGATGGAGGCACAGGAGTGGTGCAGA |
| | | AAGTACATGAAGTCTGGCAATGTGAAGGACCTGCTGCAGGCCTGGGATCT |
| | | GTACTATCACGTGTTTCGGAGAATCTCCAAGCCAGCAGCTCTCGGCAAAG |
| | | ACACGATTCCGTGGCTTGGGCATCTGCTCGTTGGGCTGAGCGGTGCGTTT |
| | | GGTTTCATCATCTTGGTCTATCTCTTGATCAATTGCAGAAATACAGGCCCT |
| | | TGGCTGAAAAAAGTGCTCAAGTGTAATACCCCCGACCCAAGCAAGTTCTT |
| | | CTCCCAGCTTTCTTCAGAGCATGGAGGCGATGTGCAGAAATGGCTCTCTT |
| | | CACCTTTTCCCTCCTCAAGCTTCTCCCCGGGAGGGCTGGCGCCCGAGATTT |
| | | CACCTCTTGAGGTACTTGAACGAGACAAGGTTACCCAACTTCTCCTTCAA |
| | | CAGGATAAGGTACCCGAACCTGCGAGCCTTAGCTTGAATACAGACGCTTA |
| | | TCTCTCACTGCAGGAACTGCAAGGATCTGGTGCTACTAATTTTTCTCTTTT |
| | | GAAGCAAGCTGGAGATGTTGAAGAGAACCCCGGTCCGGAGATGTGGCAT |
| | | GAGGGTCTGGAAGAAGCGTCTCGACTGTACTTTGGTGAGCGCAATGTGAA |
| | | GGGCATGTTTGAAGTCCTCGAACCCCTTCATGCCATGATGGAACGCGGAC |
| | | CCCAGACCTTGAAGGAGACAAGTTTTAACCAAGCTTACGGAAGAGACCT |
| | | GATGGAAGCCCAGGAATGGTGCAGGAAATACATGAAAAGCGGGAATGTG |
| | | AAGGACTTGCTCCAAGCGTGGGACCTGTACTATCATGTCTTTAGGCGCAT |
| | | TAGTAAG |
| 45 | CISCβ-DN | ATGGCACTGCCCGTGACCGCCCTGCTGCTGCCTCTGGCCCTGCTGCTGCA |
| | nucleotide | CGCAGCCCGGCCTATCCTGTGGCACGAGATGTGGCACGAGGGCCTGGAG |
| | sequence | GAGGCCAGCAGGCTGTATTTTGGCGAGCGCAACGTGAAGGGCATGTTCG |
| | | AGGTGCTGGAGCCTCTGCACGCCATGATGGAGAGAGGCCCACAGACCCT |
| | | GAAGGAGACATCCTTTAACCAGGCCTATGGACGGGACCTGATGGAGGCA |
| | | CAGGAGTGGTGCAGAAAGTACATGAAGTCTGGCAATGTGAAGGACCTGC |
| | | TGCAGGCCTGGGATCTGTACTATCACGTGTTTCGGAGAATCTCCAAGCCA |
| | | GCAGCTCTCGGCAAAGACACGATTCCGTGGCTTGGGCATCTGCTCGTTGG |
| | | GCTGAGCGGTGCGTTTGGTTTCATCATCTTGGTCTATCTCTTGATCAATTG |
| | | CAGAAATACAGGCCCTTGGCTGAAAAAAGTGCTCAAGTGTAATACCCCC |
| | | GACCCAAGCAAGTTCTTCTCCCAGCTTTCTTCAGAGCATGGAGGCGATGT |
| | | GCAGAAATGGCTCTCTTCACCTTTTCCCTCCTCAAGCTTCTCCCCGGGAGG |
| | | GCTGGCGCCCGAGATTTCACCTCTTGAGGTACTTGAACGAGACAAGGTTA |
| | | CCCAACTTCTCCTTCAACAGGATAAGGTACCCGAACCTGCGAGCCTTAGC |
| | | TCCAACCACTCTCTTACGAGCTGCTTCACCAATCAGGGATACTTCTTTTTC |
| | | CACCTTCCCGATGCGCTGGAAATCGAAGCTTGTCAAGTTTACTTTACCTAT |
| | | GATCCATATAGCGAGGAAGATCCCGACGAAGGAGTCGCCGGTGCGCCCA |
| | | CGGGTTCCTCACCCCAACCTCTCCAGCCTCTCTCAGGAGAAGATGATGCT |
| | | TATTGCACTTTTCCCAGTAGAGACGATCTCCTCCTCTTTTCTCCATCTCTTT |
| | | TGGGGGGACCTTCCCCCCCTTCTACGGCACCTGGCGGGTCTGGTGCTGGC |
| | | GAGGAGCGGATGCCGCCGTCCCTCCAGGAGCGAGTACCACGAGATTGGG |
| | | ATCCCCAGCCACTTGGACCCCCCACCCCCGGCGTACCTGACCTTGTCGAT |
| | | TTTCAACCTCCCCCTGAATTGGTGCTGCGAGAGGCTGGGGAGGAAGTTCC |
| | | GGACGCTGGGCCGAGGGAGGGCGTGTCCTTTCCATGGAGTAGGCCTCCA |
| | | GGTCAAGGCGAGTTTAGGGCTCTCAACGCGCGGCTGCCGTTGAATACAGA |
| | | CGCTTATCTCTCACTGCAGGAACTGCAAGGTCAGGACCCAACACATCTTG |
| | | TAGGATCTGGTGCTACTAATTTTTCTCTTTTGAAGCAAGCTGGAGATGTTG |
| | | AAGAGAACCCCGGTCCGGAGATGTGGCATGAGGGTCTGGAAGAAGCGTC |
| | | TCGACTGTACTTTGGTGAGCGCAATGTGAAGGGCATGTTTGAAGTCCTCG |
| | | AACCCCTTCATGCCATGATGGAACGCGGACCCCAGACCTTGAAGGAGAC |
| | | AAGTTTTAACCAAGCTTACGGAAGAGACCTGATGGAAGCCCAGGAATGG |
| | | TGCAGGAAATACATGAAAAGCGGGAATGTGAAGGACTTGCTCCAAGCGT |
| | | GGGACCTGTACTATCATGTCTTTAGGCGCATTAGTAAG |
| 46 | CISCγ- | ATGCCTCTGGGCCTGCTGTGGCTGGGCCTGGCCCTGCTGGGCGCCCTGCA |
| | FOXP3cDNA- | CGCCCAGGCCGGCGTGCAGGTGGAGACAATCTCCCCAGGCGACGGACGC |
| | LNGFR | ACATTCCCTAAGCGGGGCCAGACCTGCGTGGTGCACTATACAGGCATGCT |
| | nucleotide | GGAGGATGGCAAGAAGTTTGACAGCTCCCGGGATAGAAACAAGCCATTC |
| | sequence | AAGTTTATGCTGGGCAAGCAGGAAGTGATCAGAGGCTGGGAGGAGGGCG |
| | | TGGCCCAGATGTCTGTGGGCCAGAGGGCCAAGCTGACCATCAGCCCAGA |
| | | CTACGCCTATGGAGCAACAGGCCACCCAGGAATCATCCCACCTCACGCCA |
| | | CCCTGGTGTTCGATGTGGAGCTGCTGAAGCTGGGCGAGGGAGGGTCACCT |
| | | GGATCCAACACATCAAAAGAGAACCCCTTTCTGTTCGCATTGGAGGCCGT |
| | | AGTCATATCTGTTGGATCCATGGGACTTATTATCTCCCTGTTGTGTGTGTA |
| | | CTTCTGGCTGGAACGGACTATGCCCAGGATCCCCACGCTCAAGAATCTGG |
| | | AAGATCTCGTCACAGAATACCATGGTAATTTCAGCGCCTGGAGCGGAGTC |
| | | TCTAAGGGTCTGGCCGAATCCCTCCAACCCGATTATTCTGAACGGTTGTG |
| | | CCTCGTATCCGAAATACCACCAAAAGGCGGGGCTCTGGGTGAGGGCCCA |
| | | GGGGCGAGTCCGTGCAATCAACACAGCCCGTATTGGGCCCCTCCTTGTTA |
| | | TACGTTGAAGCCCGAAACTGGAAGCGGAGCGACTAACTTCAGCCTGCTTA |
| | | AGCAGGCCGGAGATGTGGAGGAAAACCCTGGACCGATGCCTAATCCTCG |
| | | GCCTGGAAAGCCTAGCGCTCCTTCTCTTGCTCTGGGACCTTCTCCTGGCGC |
| | | CTCTCCATCTTGGAGAGCCGCTCCTAAAGCCAGCGATCTGCTGGGAGCTA |
| | | GAGGACCTGGCGGCACATTTCAGGGCAGAGATCTTAGAGGCGGAGCCCA |
| | | CGCTAGCTCCTCCAGCCTTAATCCTATGCCTCCTAGCCAGCTCCAGCTGCC |
| | | TACACTGCCTCTGGTTATGGTGGCTCCTAGCGGAGCTAGACTGGGCCCTC |
| | | TGCCTCATCTGCAAGCTCTGCTGCAGGACAGACCCCACTTCATGCACCAG |
| | | CTGAGCACCGTGGATGCCCACGCAAGAACACCTGTGCTGCAGGTTCACCC |
| | | TCTGGAATCCCCAGCCATGATCAGCCTGACACCTCCAACAACAGCCACCG |
| | | GCGTGTTCAGCCTGAAAGCCAGACCTGGACTGCCTCCTGGCATCAATGTG |
| | | GCCAGCCTGGAATGGGTGTCCAGAGAACCTGCTCTGCTGTGCACATTCCC |
| | | CAATCCAAGCGCTCCCAGAAAGGACAGCACACTGTCTGCCGTGCCTCAGA |
| | | GCAGCTATCCCCTGCTTGCTAACGGCGTGTGCAAGTGGCCTGGATGCGAG |
| | | AAGGTGTTCGAGGAACCCGAGGACTTCCTGAAGCACTGCCAGGCCGATC |
| | | ATCTGCTGGACGAGAAAGGCAGAGCCCAGTGTCTGCTCCAGCGCGAGAT |
| | | GGTGCAGTCTCTGGAACAGCAGCTGGTCCTGGAAAAAGAAAAGCTGAGC |
| | | GCCATGCAGGCCCACCTGGCCGGAAAAATGGCCCTGACAAAGGCCAGCA |
| | | GCGTGGCCTCTTCTGATAAGGGCAGCTGCTGCATTGTGGCCGCTGGATCT |
| | | CAGGGACCTGTGGTTCCTGCTTGGAGCGGACCTAGAGAGGCCCCTGATTC |
| | | TCTGTTTGCCGTGCGGAGACACCTGTGGGGCTCTCACGGCAACTCTACTTT |
| | | CCCCGAGTTCCTGCACAACATGGACTACTTCAAGTTCCACAACATGCGGC |
| | | CTCCATTCACCTACGCCACACTGATCAGATGGGCCATTCTGGAAGCCCCT |
| | | GAGAAGCAGAGAACCCTGAACGAGATCTACCACTGGTTTACCCGGATGTT |
| | | CGCCTTCTTCCGGAATCACCCTGCCACCTGGAAGAACGCCATCCGGCACA |
| | | ATCTGAGCCTGCACAAGTGCTTCGTGCGCGTGGAATCTGAGAAAGGCGCC |
| | | GTGTGGACAGTGGACGAGCTGGAATTCAGAAAGAAGAGAAGCCAGCGGC |
| | | CTAGCCGGTGCAGCAATCCTACACCTGGACCTGGAAGCGGAGCGACTAA |
| | | CTTCAGCCTGCTGAAGCAGGCCGGAGATGTGGAGGAAAACCCTGGACCG |
| | | ATGGGGGCAGGTGCCACCGGACGAGCCATGGACGGGCCGCGCCTGCTGC |
| | | TGTTGCTGCTTCTGGGGGTGTCCCTTGGAGGTGCCAAGGAGGCATGCCCC |
| | | ACAGGCCTGTACACACACAGCGGTGAGTGCTGCAAAGCCTGCAACCTGG |
| | | GCGAGGGTGTGGCCCAGCCTTGTGGAGCCAACCAGACCGTGTGTGAGCC |
| | | CTGCCTGGACAGCGTGACGTTCTCCGACGTGGTGAGCGCGACCGAGCCGT |
| | | GCAAGCCGTGCACCGAGTGCGTGGGGCTCCAGAGCATGTCGGCGCCGTG |
| | | CGTGGAGGCCGACGACGCCGTGTGCCGCTGCGCCTACGGCTACTACCAGG |
| | | ATGAGACGACTGGGCGCTGCGAGGCGTGCCGCGTGTGCGAGGCGGGCTC |
| | | GGGCCTCGTGTTCTCCTGCCAGGACAAGCAGAACACCGTGTGCGAGGAGT |
| | | GCCCCGACGGCACGTATTCCGACGAGGCCAACCACGTGGACCCGTGCCTG |
| | | CCCTGCACCGTGTGCGAGGACACCGAGCGCCAGCTCCGCGAGTGCACAC |
| | | GCTGGGCCGACGCCGAGTGCGAGGAGATCCCTGGCCGTTGGATTACACG |
| | | GTCCACACCCCCAGAGGGCTCGGACAGCACAGCCCCCAGCACCCAGGAG |
| | | CCTGAGGCACCTCCAGAACAAGACCTCATAGCCAGCACGGTGGCAGGTG |
| | | TGGTGACCACAGTGATGGGCAGCTCCCAGCCCGTGGTGACCCGAGGCAC |
| | | CACCGACAACCTCATCCCTGTCTATTGCTCCATCCTGGCTGCTGTGGTTGT |
| | | GGGTCTTGTGGCCTACATAGCCTTCAAGAGGTGA |
| 47 | CISCγ-LNGFR- | ATGCCTCTGGGCCTGCTGTGGCTGGGCCTGGCCCTGCTGGGCGCCCTGCA |
| | FOXP3cDNA | CGCCCAGGCCGGCGTGCAGGTGGAGACAATCTCCCCAGGCGACGGACGC |
| | nucleotide | ACATTCCCTAAGCGGGGCCAGACCTGCGTGGTGCACTATACAGGCATGCT |
| | sequence | GGAGGATGGCAAGAAGTTTGACAGCTCCCGGGATAGAAACAAGCCATTC |
| | | AAGTTTATGCTGGGCAAGCAGGAAGTGATCAGAGGCTGGGAGGAGGGCG |
| | | TGGCCCAGATGTCTGTGGGCCAGAGGGCCAAGCTGACCATCAGCCCAGA |
| | | CTACGCCTATGGAGCAACAGGCCACCCAGGAATCATCCCACCTCACGCCA |
| | | CCCTGGTGTTCGATGTGGAGCTGCTGAAGCTGGGCGAGGGAGGGTCACCT |
| | | GGATCCAACACATCAAAAGAGAACCCCTTTCTGTTCGCATTGGAGGCCGT |
| | | AGTCATATCTGTTGGATCCATGGGACTTATTATCTCCCTGTTGTGTGTGTA |
| | | CTTCTGGCTGGAACGGACTATGCCCAGGATCCCCACGCTCAAGAATCTGG |
| | | AAGATCTCGTCACAGAATACCATGGTAATTTCAGCGCCTGGAGCGGAGTC |
| | | TCTAAGGGTCTGGCCGAATCCCTCCAACCCGATTATTCTGAACGGTTGTG |
| | | CCTCGTATCCGAAATACCACCAAAAGGCGGGGCTCTGGGTGAGGGCCCA |
| | | GGGGCGAGTCCGTGCAATCAACACAGCCCGTATTGGGCCCCTCCTTGTTA |
| | | TACGTTGAAGCCCGAAACTGGAAGCGGAGCGACTAACTTCAGCCTGCTTA |
| | | AGCAGGCCGGAGATGTGGAGGAAAACCCTGGACCGATGGGGGCAGGTGC |
| | | CACCGGACGAGCCATGGACGGGCCGCGCCTGCTGCTGTTGCTGCTTCTGG |
| | | GGGTGTCCCTTGGAGGTGCCAAGGAGGCATGCCCCACAGGCCTGTACAC |
| | | ACACAGCGGTGAGTGCTGCAAAGCCTGCAACCTGGGCGAGGGTGTGGCC |
| | | CAGCCTTGTGGAGCCAACCAGACCGTGTGTGAGCCCTGCCTGGACAGCGT |
| | | GACGTTCTCCGACGTGGTGAGCGCGACCGAGCCGTGCAAGCCGTGCACC |
| | | GAGTGCGTGGGGCTCCAGAGCATGTCGGCGCCGTGCGTGGAGGCCGACG |
| | | ACGCCGTGTGCCGCTGCGCCTACGGCTACTACCAGGATGAGACGACTGGG |
| | | CGCTGCGAGGCGTGCCGCGTGTGCGAGGCGGGCTCGGGCCTCGTGTTCTC |
| | | CTGCCAGGACAAGCAGAACACCGTGTGCGAGGAGTGCCCCGACGGCACG |
| | | TATTCCGACGAGGCCAACCACGTGGACCCGTGCCTGCCCTGCACCGTGTG |
| | | CGAGGACACCGAGCGCCAGCTCCGCGAGTGCACACGCTGGGCCGACGCC |
| | | GAGTGCGAGGAGATCCCTGGCCGTTGGATTACACGGTCCACACCCCCAGA |
| | | GGGCTCGGACAGCACAGCCCCCAGCACCCAGGAGCCTGAGGCACCTCCA |
| | | GAACAAGACCTCATAGCCAGCACGGTGGCAGGTGTGGTGACCACAGTGA |
| | | TGGGCAGCTCCCAGCCCGTGGTGACCCGAGGCACCACCGACAACCTCATC |
| | | CCTGTCTATTGCTCCATCCTGGCTGCTGTGGTTGTGGGTCTTGTGGCCTAC |
| | | ATAGCCTTCAAGAGGGGAAGCGGAGCGACTAACTTCAGCCTGCTGAAGC |
| | | AGGCCGGAGATGTGGAGGAAAACCCTGGACCGATGCCTAATCCTCGGCC |
| | | TGGAAAGCCTAGCGCTCCTTCTCTTGCTCTGGGACCTTCTCCTGGCGCCTC |
| | | TCCATCTTGGAGAGCCGCTCCTAAAGCCAGCGATCTGCTGGGAGCTAGAG |
| | | GACCTGGCGGCACATTTCAGGGCAGAGATCTTAGAGGCGGAGCCCACGC |
| | | TAGCTCCTCCAGCCTTAATCCTATGCCTCCTAGCCAGCTCCAGCTGCCTAC |
| | | ACTGCCTCTGGTTATGGTGGCTCCTAGCGGAGCTAGACTGGGCCCTCTGC |
| | | CTCATCTGCAAGCTCTGCTGCAGGACAGACCCCACTTCATGCACCAGCTG |
| | | AGCACCGTGGATGCCCACGCAAGAACACCTGTGCTGCAGGTTCACCCTCT |
| | | GGAATCCCCAGCCATGATCAGCCTGACACCTCCAACAACAGCCACCGGC |
| | | GTGTTCAGCCTGAAAGCCAGACCTGGACTGCCTCCTGGCATCAATGTGGC |
| | | CAGCCTGGAATGGGTGTCCAGAGAACCTGCTCTGCTGTGCACATTCCCCA |
| | | ATCCAAGCGCTCCCAGAAAGGACAGCACACTGTCTGCCGTGCCTCAGAGC |
| | | AGCTATCCCCTGCTTGCTAACGGCGTGTGCAAGTGGCCTGGATGCGAGAA |
| | | GGTGTTCGAGGAACCCGAGGACTTCCTGAAGCACTGCCAGGCCGATCATC |
| | | TGCTGGACGAGAAAGGCAGAGCCCAGTGTCTGCTCCAGCGCGAGATGGT |
| | | GCAGTCTCTGGAACAGCAGCTGGTCCTGGAAAAAGAAAAGCTGAGCGCC |
| | | ATGCAGGCCCACCTGGCCGGAAAAATGGCCCTGACAAAGGCCAGCAGCG |
| | | TGGCCTCTTCTGATAAGGGCAGCTGCTGCATTGTGGCCGCTGGATCTCAG |
| | | GGACCTGTGGTTCCTGCTTGGAGCGGACCTAGAGAGGCCCCTGATTCTCT |
| | | GTTTGCCGTGCGGAGACACCTGTGGGGCTCTCACGGCAACTCTACTTTCC |
| | | CCGAGTTCCTGCACAACATGGACTACTTCAAGTTCCACAACATGCGGCCT |
| | | CCATTCACCTACGCCACACTGATCAGATGGGCCATTCTGGAAGCCCCTGA |
| | | GAAGCAGAGAACCCTGAACGAGATCTACCACTGGTTTACCCGGATGTTCG |
| | | CCTTCTTCCGGAATCACCCTGCCACCTGGAAGAACGCCATCCGGCACAAT |
| | | CTGAGCCTGCACAAGTGCTTCGTGCGCGTGGAATCTGAGAAAGGCGCCGT |
| | | GTGGACAGTGGACGAGCTGGAATTCAGAAAGAAGAGAAGCCAGCGGCCT |
| | | AGCCGGTGCAGCAATCCTACACCTGGACCTTGA |
| 48 | IL2Rγ-CISC | MPLGLLWLGLALLGALHAQAGVQVETISPGDGRTFPKRGQTCVVHYTGMLE |
| | amino acid | DGKKFDSSRDRNKPFKFMLGKQEVIRGWEEGVAQMSVGQRAKLTISPDYAY |
| | sequence | GATGHPGIIPPHATLVFDVELLKLGEGSNTSKENPFLFALEAVVISVGSMGLII |
| | | SLLCVYFWLERTMPRIPTLKNLEDLVTEYHGNFSAWSGVSKGLAESLQPDYS |
| | | ERLCLVSEIPPKGGALGEGPGASPCNQHSPYWAPPCYTLKPET |
| 49 | IL2Rβ-CISC | MALPVTALLLPLALLLHAARPILWHEMWHEGLEEASRLYFGERNVKGMFEV |
| | | LEPLHAMMERGPQTLKETSFNQAYGRDLMEAQEWCRKYMKSGNVKDLLQ |
| | | AWDLYYHVFRRISKGKDTIPWLGHLLVGLSGAFGFIILVYLLINCRNTGPWL |
| | | KKVLKCNTPDPSKFFSQLSSEHGGDVQKWLSSPFPSSSFSPGGLAPEISPLEVL |
| | | ERDKVTQLLLQQDKVPEPASLSSNHSLTSCFTNQGYFFFHLPDALEIEACQVY |
| | | FTYDPYSEEDPDEGVAGAPTGSSPQPLQPLSGEDDAYCTFPSRDDLLLFSPSLL |
| | | GGPSPPSTAPGGSGAGEERMPPSLQERVPRDWDPQPLGPPTPGVPDLVDFQPP |
| | | PELVLREAGEEVPDAGPREGVSFPWSRPPGQGEFRALNARLPLNTDAYLSLQ |
| | | ELQGQDPTHLV |
| 50 | IL2Rγ-CISC | MPLGLLWLGLALLGALHAQAGVQVETISPGDGRTFPKRGQTCVVHYTGMLE |
| | | DGKKFDSSRDRNKPFKFMLGKQEVIRGWEEGVAQMSVGQRAKLTISPDYAY |
| | | GATGHPGIIPPHATLVFDVELLKLEGGGSQNLVIPWAPENLTLHKLSESQLEL |
| | | NWNNRFLNHCLEHLVQYRTDWDHSWTEQSVDYRHKFSLPSVDGQKRYTFR |
| | | VRSRFNPLCGSAQHWSEWSHPIHWGSNTSKENPFLFALEAVVISVGSMGLIIS |
| | | LLCVYFWLERTMPRIPTLKNLEDLVTEYHGNFSAWSGVSKGLAESLQPDYSE |
| | | RLCLVSEIPPKGGALGEGPGASPCNQHSPYWAPPCYTLKPET |
| 51 | IL2Rβ-CISC | MALPVTALLLPLALLLHAARPILWHEMWHEGLEEASRLYFGERNVKGMFEV |
| | | LEPLHAMMERGPQTLKETSFNQAYGRDLMEAQEWCRKYMKSGNVKDLLQ |
| | | AWDLYYHVFRRISKGGSKPFENLRLMAPISLQVVHVETHRCNISWEISQASH |
| | | YFERHLEFEARTLSPGHTWEEAPLLTLKQKQEWICLETLTPDTQYEFQVRVK |
| | | PLQGEFTTWSPWSQPLAFRTKPAALGKDTIPWLGHLLVGLSGAFGFIILVYLL |
| | | INCRNTGPWLKKVLKCNTPDPSKFFQLSSEHGGDVQKWLSSPFPSSSFSPGGL |
| | | APEISPLEVLERDKVTQLLLQQDKVPEPASLSSNHSLTSCFTNQGYFFFHLPDA |
| | | LEIEACQVYFTYDPYSEEDPDEGVAGAPTGSSPQPLQPLSGEDDAYCTFPSRD |
| | | DLLLFSPSLLGGPSPPSTAPGGSGAGEERMPPSLQERVPRDWDPQPLGPPTPG |
| | | VPDLVDFQPPPELVLREAGEEVPDAGPREGVSFPWSRPPGQGEFRALNARLP |
| | | LNTDAYLSLQELQGQDPTHLV |
| 52 | IL2Rγ-CISC | MPLGLLWLGLALLGALHAQAGVQVETISPGDGRTFPKRGQTCVVHYTGMLE |
| | | DGKKFDSSRDRNKPFKFMLGKQEVIRGWEEGVAQMSVGQRAKLTISPDYAY |
| | | GATGHPGIIPPHATLVFDVELLKLEGQNLVIPWAPENLTLHKLSESQLELNWN |
| | | NRFLNHCLEHLVQYRTDWDHSWTEQSVDYRHKFSLPSVDGQKRYTFRVRSR |
| | | FNPLCGSAQHWSEWSHPIHWGSNTSKENPFLFALEAVVISVGSMGLIISLLCV |
| | | YFWLERTMPRIPTLKNLEDLVTEYHGNFSAWSGVSKGLAESLQPDYSERLCL |
| | | VSEIPPKGGALGEGPGASPCNQHSPYWAPPCYTLKPET |
| 53 | IL2Rβ-CISC | MALPVTALLLPLALLLHAARPILWHEMWHEGLEEASRLYFGERNVKGMFEV |
| | | LEPLHAMMERGPQTLKETSFNQAYGRDLMEAQEWCRKYMKSGNVKDLLQ |
| | | AWDLYYHVFRRISKKPFENLRLMAPISLQVVHVETHRCNISWEISQASHYFER |
| | | HLEFEARTLSPGHTWEEAPLLTLKQKQEWICLETLTPDTQYEFQVRVKPLQG |
| | | EFTTWSPWSQPLAFRTKPAALGKDTIPWLGHLLVGLSGAFGFIILVYLLINCR |
| | | NTGPWLKKVLKCNTPDPSKFFSQLSSEHGGDVQKWLSSPFPSSSFSPGGLAPE |
| | | ISPLEVLERDKVTQLLLQQDKVPEPASLSSNHSLTSCFTNQGYFFFHLPDALEI |
| | | EACQVYFTYDPYSEEDPDEGVAGAPTGSSPQPLQPLSGEDDAYCTFPSRDDL |
| | | LLFSPSLLGGPSPPSTAPGGSGAGEERMPPSLQERVPRDWDPQPLGPPTPGVP |
| | | DLVDFQPPPELVLREAGEEVPDAGPREGVSFPWSRPPGQGEFRALNARLPLN |
| | | TDAYLSLQELQGQDPTHLV |
| 54 | IL2Rγ-CISC | MPLGLLWLGLALLGALHAQAGVQVETISPGDGRTFPKRGQTCVVHYTGMLE |
| | | DGKKFDSSRDRNKPFKFMLGKQEVIRGWEEGVAQMSVGQRAKLTISPDYAY |
| | | GATGHPGIIPPHATLVFDVELLKLEGGSNTSKENPFLFALEAVVISVGSMGLII |
| | | SLLCVYFWLERTMPRIPTLKNLEDLVTEYHGNFSAWSGVSKGLAESLQPDYS |
| | | ERLCLVSEIPPKGGALGEGPGASPCNQHSPYWAPPCYTLKPET |
| 55 | IL2Rβ-CISC | MALPVTALLLPLALLLHAARPILWHEMWHEGLEEASRLYFGERNVKGMFEV |
| | | LEPLHAMMERGPQTLKETSWLGHLLVGLSGAFGFIILVYLLINCRNTGPWLK |
| | | KVLKCNTPDPSKFFSQLSSEHGGDVQKWLSSPFPSSSFSPGGLAPEISPLEVLE |
| | | RDKVTQLLLQQDKVPEPASLSSNHSLTSCFTNQGYFFFHLPDALEIEACQVYF |
| | | TYDPYSEEDPDEGVAGAPTGSSPQPLQPLSGEDDAYCTFPSRDDLLLFSPSLL |
| | | GGPSPPSTAPGGSGAGEERMPPSLQERVPRDWDPQPLGPPTPGVPDLVDFQPP |
| | | PELVLREAGEEVPDAGPREGVSFPWSRPPGQGEFRALNARLPLNTDAYLSLQ |
| | | ELQGQDPTHLV |
| 56 | IL7Rα-CISC | MALPVTALLLPLALLLHAARPILWHEMWHEGLEEASRLYFGERNVKGMFEV |
| | | LEPLHAMMERGPQTLKETSFNQAYGRDLMEAQEWCRKYMKSGNVKDLLQ |
| | | AWDLYYHVFRRISKGEINNSSGEMDPILLTISILSFFSVALLVILACVLWKKRI |
| | | KPIVWPSLPDHKKTLEHLCKKPRKNLNVSFNPESFLDCQIHRVDDIQARDEVE |
| | | GFLQDTFPQQLEESEKQRLGGDVQSPNCPSEDVVITPESFGRDSSLTCLAGNV |
| | | SACDAPILSSSRSLDCRESGKNGPHVYQDLLLSLGTTNSTLPPPFSLQSGILTL |
| | | NPVAQGQPILTSLGSNQEEAYVTMSSFYQNQ |
| 57 | IL2Rβ-CISC | MPLGLLWLGLALLGALHAQAGVQVETISPGDGRTFPKRGQTCVVHYTGMLE |
| | | DGKKVDSSRDRNKPFKFMLGKQEVIRGWEEGVAQMSVGQRAKLTISPDYA |
| | | YGATGHPGIIPPHATLVFDVELLKLEGGKDTIPWLGHLLVGLSGAFGFIILVYL |
| | | LINCRNTGPWLKKVLKCNTPDPSKFFSQLSSEHGGDVQKWLSSPFPSSSFSPG |
| | | GLAPEISPLEVLERDKVTQLLLQQDKVPEPASLSSNHSLTSCFTNQGYFFFHLP |
| | | DALEIEACQVYFTYDPYSEEDPDEGVAGAPTGSSPQPLQPLSGEDDAYCTFPS |
| | | RDDLLLFSPSLLGGPSPPSTAPGGSGAGEERMPPSLQERVPRDWDPQPLGPPT |
| | | PGVPDLVDFQPPPELVLREAGEEVPDAGPREGVSFPWSRPPGQGEFRALNAR |
| | | LPLNTDAYLSLQELQGQDPTHLV |
| 58 | IL2Rγ-CISC | MPLGLLWLGLALLGALHAQAGVQVETISPGDGRTFPKRGQTCVVHYTGMLE |
| | | DGKKVDSSRDRNKPFKFMLGKQEVIRGWEEGVAQMSVGQRAKLTISPDYA |
| | | YGATGHPGIIPPHATLVFDVELLKLEGGSNTSKENPFLFALEAVVISVGSMGLI |
| | | ISLLCVYFWLERTMPRIPTLKNLEDLVTEYHGNFSAWSGVSKGLAESLQPDY |
| | | SERLCLVSEIPPKGGALGEGPGASPCNQHSPYWAPPCYTLKPET |
| 59 | IL2Rα-CISC | MPLGLLWLGLALLGALHAQAGVQVETISPGDGRTFPKRGQTCVVHYTGMLE |
| | | DGKKVDSSRDRNKPFKFMLGKQEVIRGWEEGVAQMSVGQRAKLTISPDYA |
| | | YGATGHPGIIPPHATLVFDVELLKLEGEINNSSGEMDPILLTISILSFFSVALLVI |
| | | LACVLWKKRIKPIVWPSLPDHKKTLEHLCKKPRKNLNVSFNPESFLDCQIHR |
| | | VDDIQARDEVEGFLQDTFPQQLEESEKQRLGGDVQSPNCPSEDVVITPESFGR |
| | | DSSLTCLAGNVSACDAPILSSSRSLDCRESGKNGPHVYQDLLLSLGTTNSTLP |
| | | PPFSLQSGILTLNPVAQGQPILTSLGSNQEEAYVTMSSFYQNQ |
| 60 | IL7Rα-CISC | MPLGLLWLGLALLGALHAQAGVQVETISPGDGRTFPKRGQTCVVHYTGMLE |
| | | DGKKVDSSRDRNKPFKFMLGKQEVIRGWEEGVAQMSVGQRAKLTISPDYA |
| | | YGATGHPGIIPPHATLVFDVELLKLEGEINNSSGEMDPILLTISILSFFSVALLVI |
| | | LACVLWKKRIKPIVWPSLPDHKKTLEHLCKKPRKNLNVSFNPESFLDCQIHR |
| | | VDDIQARDEVEGFLQDTFPQQLEESEKQRLGGDVQSPNCPSEDVVITPESFGR |
| | | DSSLTCLAGNVSACDAPILSSSRSLDCRESGKNGPHVYQDLLLSLGTTNSTLP |
| | | PPFSLQSGILTLNPVAQGQPILTSLGSNQEEAYVTMSSFYQNQ |
| 61 | MPL-CISC | MPLGLLWLGLALLGALHAQAGVQVETISPGDGRTFPKRGQTCVVHYTGMLE |
| | | DGKKVDSSRDRNKPFKFMLGKQEVIRGWEEGVAQMSVGQRAKLTISPDYA |
| | | YGATGHPGIIPPHATLVFDVELLKLGEETAWISLVTALHLVLGLSAVLGLLLL |
| | | RWQFPAHYRRLRHALWPSLPDLHRVLGQYLRDTAALSPPKATVSDTCEEVE |
| | | PSLLEILPKSSERTPLPLCSSQAQMDYRRLQPSCLGTMPLSVCPPMAESGSCCT |
| | | THIANHSYLPLSYWQQP |
| 62 | glycine amino | GGGS |
| | acid spacer | |
| 63 | glycine amino | GGGSGGG |
| | acid spacer | |
| 64 | glycine amino | GGG |
| | acid spacer | |
| 65 | expression | AGCTTAATGTAGTCTTATGCAATACTCTTGTAGTCTTGCAACATGGTAACG |
| | vector | ATGAGTTAGCAACATGCCTTACAAGGAGAGAAAAAGCACCGTGCATGCC |
| | | GATTGGTGGAAGTAAGGTGGTACGATCGTGCCTTATTAGGAAGGCAACA |
| | | GACGGGTCTGACATGGATTGGACGAACCACTGAATTGCCGCATTGCAGA |
| | | GATATTGTATTTAAGTGCCTAGCTCGATACAATAAACGGGTCTCTCTGGTT |
| | | AGACCAGATCTGAGCCTGGGAGCTCTCTGGCTAACTAGGGAACCCACTGC |
| | | TTAAGCCTCAATAAAGCTTGCCTTGAGTGCTTCAAGTAGTGTGTGCCCGT |
| | | CTGTTGTGTGACTCTGGTAACTAGAGATCCCTCAGACCCTTTTAGTCAGTG |
| | | TGGAAAATCTCTAGCAGTGGCGCCCGAACAGGGACTTGAAAGCGAAAGG |
| | | GAAACCAGAGGAGCTCTCTCGACGCAGGACTCGGCTTGCTGAAGCGCGC |
| | | ACGGCAAGAGGCGAGGGGCGGCGACTGGTGAGTACGCCAAAAATTTTGA |
| | | CTAGCGGAGGCTAGAAGGAGAGAGATGGGTGCGAGAGCGTCAGTATTAA |
| | | GCGGGGGAGAATTAGATCGCGATGGGAAAAAATTCGGTTAAGGCCAGGG |
| | | GGAAAGAAAAAATATAAATTAAAACATATAGTATGGGCAAGCAGGGAGC |
| | | TAGAACGATTCGCAGTTAATCCTGGCCTGTTAGAAACATCAGAAGGCTGT |
| | | AGACAAATACTGGGACAGCTACAACCATCCCTTCAGACAGGATCAGAAG |
| | | AACTTAGATCATTATATAATACAGTAGCAACCCTCTATTGTGTGCATCAA |
| | | AGGATAGAGATAAAAGACACCAAGGAAGCTTTAGACAAGATAGAGGAA |
| | | GAGCAAAACAAAAGTAAGACCACCGCACAGCAAGCGGCCGCTGATCTTC |
| | | AGACCTGGAGGAGGAGATATGAGGGACAATTGGAGAAGTGAATTATATA |
| | | AATATAAAGTAGTAAAAATTGAACCATTAGGAGTAGCACCCACCAAGGC |
| | | AAAGAGAAGAGTGGTGCAGAGAGAAAAAAGAGCAGTGGGAATAGGAGC |
| | | TTTGTTCCTTGGGTTCTTGGGAGCAGCAGGAAGCACTATGGGCGCAGCCT |
| | | CAATGACGCTGACGGTACAGGCCAGACAATTATTGTCTGGTATAGTGCAG |
| | | CAGCAGAACAATTTGCTGAGGGCTATTGAGGCGCAACAGCATCTGTTGCA |
| | | ACTCACAGTCTGGGGCATCAAGCAGCTCCAGGCAAGAATCCTGGCTGTGG |
| | | AAAGATACCTAAAGGATCAACAGCTCCTGGGGATTTGGGGTTGCTCTGGA |
| | | AAACTCATTTGCACCACTGCTGTGCCTTGGAATGCTAGTTGGAGTAATAA |
| | | ATCTCTGGAACAGATTTGGAATCACACGACCTGGATGGAGTGGGACAGA |
| | | GAAATTAACAATTACACAAGCTTAATACACTCCTTAATTGAAGAATCGCA |
| | | AAACCAGCAAGAAAAGAATGAACAAGAATTATTGGAATTAGATAAATGG |
| | | GCAAGTTTGTGGAATTGGTTTAACATAACAAATTGGCTGTGGTATATAAA |
| | | ATTATTCATAATGATAGTAGGAGGCTTGGTAGGTTTAAGAATAGTTTTTG |
| | | CTGTACTTTCTATAGTGAATAGAGTTAGGCAGGGATATTCACCATTATCG |
| | | TTTCAGACCCACCTCCCAACCCCGAGGGGACCCGACAGGCCCGAAGGAA |
| | | TAGAAGAAGAAGGTGGAGAGAGAGACAGAGACAGATCCATTCGATTAGT |
| | | GAACGGATCTCGACGGTATCGGTTAACTTTTAAAAGAAAAGGGGGGATT |
| | | GGGGGGTACAGTGCAGGGGAAAGAATAGTAGACATAATAGCAACAGAC |
| | | ATACAAACTAAAGAATTACAAAAACAAATTACAAAAATTCAAAATTTTAT |
| | | CGATCACGAGACTAGCCTCGAGAAGCTTGATATCGAATTCCCACGGGGTT |
| | | GGACGCGTAGGAACAGAGAAACAGGAGAATATGGGCCAAACAGGATAT |
| | | CTGTGGTAAGCAGTTCCTGCCCCGGCTCAGGGCCAAGAACAGTTGGAACA |
| | | GCAGAATATGGGCCAAACAGGATATCTGTGGTAAGCAGTTCCTGCCCCGG |
| | | CTCAGGGCCAAGAACAGATGGTCCCCAGATGCGGTCCCGCCCTCAGCAGT |
| | | TTCTAGAGAACCATCAGATGTTTCCAGGGTGCCCCAAGGACCTGAAATGA |
| | | CCCTGTGCCTTATTTGAACTAACCAATCAGTTCGCTTCTCGCTTCTGTTCG |
| | | CGCGCTTCTGCTCCCCGAGCTCTATATAAGCAGAGCTCGTTTAGTGAACC |
| | | GTCAGATCGCTAGCACCGGTGCCGCCACCATGCCTCTGGGCCTGCTGTGG |
| | | CTGGGCCTGGCCCTGCTGGGCGCCCTGCACGCCCAGGCCGGCGTGCAGGT |
| | | GGAGACAATCTCCCCAGGCGACGGACGCACATTCCCTAAGCGGGGCCAG |
| | | ACCTGCGTGGTGCACTATACAGGCATGCTGGAGGATGGCAAGAAGTTTG |
| | | ACAGCTCCCGGGATAGAAACAAGCCATTCAAGTTTATGCTGGGCAAGCA |
| | | GGAAGTGATCAGAGGCTGGGAGGAGGGCGTGGCCCAGATGTCTGTGGGC |
| | | CAGAGGGCCAAGCTGACCATCAGCCCAGACTACGCCTATGGAGCAACAG |
| | | GCCACCCAGGAATCATCCCACCTCACGCCACCCTGGTGTTCGATGTGGAG |
| | | CTGCTGAAGCTGGGCGAGGGCGGTAGTCAGAACCTTGTGATACCATGGG |
| | | CCCCAGAAAATCTCACACTTCATAAACTTTCCGAATCACAACTCGAACTC |
| | | AACTGGAATAACCGGTTCCTGAATCACTGTCTTGAACACCTGGTACAATA |
| | | TCGGACCGACTGGGATCACTCATGGACAGAACAATCTGTGGACTATAGGC |
| | | ACAAATTCTCACTCCCAAGCGTAGACGGCCAAAAAAGATACACTTTTCGC |
| | | GTACGATCCCGCTTTAATCCTCTCTGCGGCTCTGCTCAGCACTGGAGTGA |
| | | ATGGTCCCATCCCATTCATTGGGGATCCAACACATCAAAAGAGAACCCCT |
| | | TTCTGTTCGCATTGGAGGCCGTAGTCATATCTGTTGGATCCATGGGACTTA |
| | | TTATCTCCCTGTTGTGTGTGTACTTCTGGCTGGAACGGACTATGCCCAGGA |
| | | TCCCCACGCTCAAGAATCTGGAAGATCTCGTCACAGAATACCATGGTAAT |
| | | TTCAGCGCCTGGAGCGGAGTCTCTAAGGGTCTGGCCGAATCCCTCCAACC |
| | | CGATTATTCTGAACGGTTGTGCCTCGTATCCGAAATACCACCAAAAGGCG |
| | | GGGCTCTGGGTGAGGGCCCAGGGGCGAGTCCGTGCAATCAACACAGCCC |
| | | GTATTGGGCCCCTCCTTGTTATACGTTGAAGCCCGAAACTGGAAGCGGAG |
| | | CTACTAACTTCAGCCTGCTGAAGCAGGCTGGAGACGTGGAGGAGAACCC |
| | | TGGACCTATGGCACTGCCCGTGACCGCCCTGCTGCTGCCTCTGGCCCTGCT |
| | | GCTGCACGCAGCCCGGCCTATCCTGTGGCACGAGATGTGGCACGAGGGC |
| | | CTGGAGGAGGCCAGCAGGCTGTATTTTGGCGAGCGCAACGTGAAGGGCA |
| | | TGTTCGAGGTGCTGGAGCCTCTGCACGCCATGATGGAGAGAGGCCCACA |
| | | GACCCTGAAGGAGACATCCTTTAACCAGGCCTATGGACGGGACCTGATG |
| | | GAGGCACAGGAGTGGTGCAGAAAGTACATGAAGTCTGGCAATGTGAAGG |
| | | ACCTGCTGCAGGCCTGGGATCTGTACTATCACGTGTTTCGGAGAATCTCC |
| | | AAGGGAGGTTCAAAACCTTTTGAGAACCTTAGACTGATGGCGCCCATCTC |
| | | TCTGCAGGTAGTTCACGTTGAGACCCATAGATGCAATATAAGCTGGGAAA |
| | | TCTCACAAGCCAGCCATTACTTTGAACGGCATTTGGAATTCGAGGCCCGA |
| | | ACACTTTCCCCCGGTCATACGTGGGAAGAAGCTCCTCTCTTGACGCTGAA |
| | | GCAGAAGCAGGAGTGGATTTGTCTGGAGACTTTGACTCCTGATACTCAGT |
| | | ATGAGTTCCAAGTTCGGGTGAAACCACTCCAAGGCGAGTTCACGACGTGG |
| | | TCTCCGTGGAGTCAACCGTTGGCGTTCCGCACGAAGCCCGCTGCCCTTGG |
| | | CAAAGACACGATTCCGTGGCTTGGGCATCTGCTCGTTGGGCTGAGTGGTG |
| | | CGTTTGGTTTCATCATCTTGGTCTATCTCTTGATCAATTGCAGAAATACAG |
| | | GCCCTTGGCTGAAAAAAGTGCTCAAGTGTAATACCCCCGACCCAAGCAA |
| | | GTTCTTCTCCCAGCTTTCTTCAGAGCATGGAGGCGATGTGCAGAAATGGC |
| | | TCTCTTCACCTTTTCCCTCCTCAAGCTTCTCCCCGGGAGGGCTGGCGCCCG |
| | | AGATTTCACCTCTTGAGGTACTTGAACGAGACAAGGTTACCCAACTTCTC |
| | | CTTCAACAGGATAAGGTACCCGAACCTGCGAGCCTTAGCTCCAACCACTC |
| | | TCTTACGAGCTGCTTCACCAATCAGGGATACTTCTTTTTCCACCTTCCCGA |
| | | TGCGCTGGAAATCGAAGCTTGTCAAGTTTACTTTACCTATGATCCATATA |
| | | GCGAGGAAGATCCCGACGAAGGAGTCGCCGGTGCGCCCACGGGTTCCTC |
| | | ACCCCAACCTCTCCAGCCTCTCTCAGGAGAAGATGATGCTTATTGCACTTT |
| | | TCCCAGTAGAGACGATCTCCTCCTCTTTTCTCCATCTCTTTTGGGGGGACC |
| | | TTCCCCCCCTTCTACGGCACCTGGCGGGTCTGGTGCTGGCGAGGAGCGGA |
| | | TGCCGCCGTCCCTCCAGGAGCGAGTACCACGAGATTGGGATCCCCAGCCA |
| | | CTTGGACCCCCCACCCCCGGCGTACCTGACCTTGTCGATTTTCAACCTCCC |
| | | CCTGAATTGGTGCTGCGAGAGGCTGGGGAGGAAGTTCCGGACGCTGGGC |
| | | CGAGGGAGGGCGTGTCCTTTCCATGGAGTAGGCCTCCAGGTCAAGGCGA |
| | | GTTTAGGGCTCTCAACGCGCGGCTGCCGTTGAATACAGACGCTTATCTCT |
| | | CACTGCAGGAACTGCAAGGTCAGGACCCAACACATCTTGTAGGATCTGGT |
| | | GCTACTAATTTTTCTCTTTTGAAGCAAGCTGGAGATGTTGAAGAGAACCC |
| | | TGGTCCAGTGAGCAAGGGCGAGGAGCTGTTCACCGGGGTGGTGCCCATC |
| | | CTGGTCGAGCTGGACGGCGACGTAAACGGCCACAAGTTCAGCGTGTCCG |
| | | GCGAGGGCGAGGGCGATGCCACCTACGGCAAGCTGACCCTGAAGTTCAT |
| | | CTGCACCACCGGCAAGCTGCCCGTGCCCTGGCCCACCCTCGTGACCACCC |
| | | TGACCTACGGCGTGCAGTGCTTCAGCCGCTACCCCGACCACATGAAGCAG |
| | | CACGACTTCTTCAAGTCCGCCATGCCCGAAGGCTACGTCCAGGAGCGCAC |
| | | CATCTTCTTCAAGGACGACGGCAACTACAAGACCCGCGCCGAGGTGAAG |
| | | TTCGAGGGCGACACCCTGGTGAACCGCATCGAGCTGAAGGGCATCGACTT |
| | | CAAGGAGGACGGCAACATCCTGGGGCACAAGCTGGAGTACAACTACAAC |
| | | AGCCACAACGTCTATATCATGGCCGACAAGCAGAAGAACGGCATCAAGG |
| | | TGAACTTCAAGATCCGCCACAACATCGAGGACGGCAGCGTGCAGCTCGC |
| | | CGACCACTACCAGCAGAACACCCCCATCGGCGACGGCCCCGTGCTGCTGC |
| | | CCGACAACCACTACCTGAGCACCCAGTCCGCCCTGAGCAAAGACCCCAA |
| | | CGAGAAGCGCGATCACATGGTCCTGCTGGAGTTCGTGACCGCCGCCGGG |
| | | ATCACTCTCGGCATGGACGAGCTGTACAAGTAAACTAGTGTCGACAATCA |
| | | ACCTCTGGATTACAAAATTTGTGAAAGATTGACTGGTATTCTTAACTATGT |
| | | TGCTCCTTTTACGCTATGTGGATACGCTGCTTTAATGCCTTTGTATCATGC |
| | | TATTGCTTCCCGTATGGCTTTCATTTTCTCCTCCTTGTATAAATCCTGGTTG |
| | | CTGTCTCTTTATGAGGAGTTGTGGCCCGTTGTCAGGCAACGTGGCGTGGT |
| | | GTGCACTGTGTTTGCTGACGCAACCCCCACTGGTTGGGGCATTGCCACCA |
| | | CCTGTCAGCTCCTTTCCGGGACTTTCGCTTTCCCCCTCCCTATTGCCACGG |
| | | CGGAACTCATCGCCGCCTGCCTTGCCCGCTGCTGGACAGGGGCTCGGCTG |
| | | TTGGGCACTGACAATTCCGTGGTGTTGTCGGGGAAGCTGACGTCCTTTCC |
| | | ATGGCTGCTCGCCTGTGTTGCCACCTGGATTCTGCGCGGGACGTCCTTCTG |
| | | CTACGTCCCTTCGGCCCTCAATCCAGCGGACCTTCCTTCCCGCGGCCTGCT |
| | | GCCGGCTCTGCGGCCTCTTCCGCGTCTTCGCCTTCGCCCTCAGACGAGTCG |
| | | GATCTCCCTTTGGGCCGCCTCCCCGCCTGGAATTCGAGCTCGGTACCTTTA |
| | | AGACCAATGACTTACAAGGCAGCTGTAGATCTTAGCCACTTTTTAAAAGA |
| | | AAAGGGGGGACTGGAAGGGCTAATTCACTCCCAACGAAGACAAGATCTG |
| | | CTTTTTGCTTGTACTGGGTCTCTCTGGTTAGACCAGATCTGAGCCTGGGAG |
| | | CTCTCTGGCTAACTAGGGAACCCACTGCTTAAGCCTCAATAAAGCTTGCC |
| | | TTGAGTGCTTCAAGTAGTGTGTGCCCGTCTGTTGTGTGACTCTGGTAACTA |
| | | GAGATCCCTCAGACCCTTTTAGTCAGTGTGGAAAATCTCTAGCAGTAGTA |
| | | GTTCATGTCATCTTATTATTCAGTATTTATAACTTGCAAAGAAATGAATAT |
| | | CAGAGAGTGAGAGGAACTTGTTTATTGCAGCTTATAATGGTTACAAATAA |
| | | AGCAATAGCATCACAAATTTCACAAATAAAGCATTTTTTTCACTGCATTCT |
| | | AGTTGTGGTTTGTCCAAACTCATCAATGTATCTTATCATGTCTGGCTCTAG |
| | | CTATCCCGCCCCTAACTCCGCCCAGTTCCGCCCATTCTCCGCCCCATGGCT |
| | | GACTAATTTTTTTTATTTATGCAGAGGCCGAGGCCGCCTCGGCCTCTGAGC |
| | | TATTCCAGAAGTAGTGAGGAGGCTTTTTTGGAGGCCTAGGCTTTTGCGTC |
| | | GAGACGTACCCAATTCGCCCTATAGTGAGTCGTATTACGCGCGCTCACTG |
| | | GCCGTCGTTTTACAACGTCGTGACTGGGAAAACCCTGGCGTTACCCAACT |
| | | TAATCGCCTTGCAGCACATCCCCCTTTCGCCAGCTGGCGTAATAGCGAAG |
| | | AGGCCCGCACCGATCGCCCTTCCCAACAGTTGCGCAGCCTGAATGGCGAA |
| | | TGGCGCGACGCGCCCTGTAGCGGCGCATTAAGCGCGGCGGGTGTGGTGG |
| | | TTACGCGCAGCGTGACCGCTACACTTGCCAGCGCCCTAGCGCCCGCTCCT |
| | | TTCGCTTTCTTCCCTTCCTTTCTCGCCACGTTCGCCGGCTTTCCCCGTCAAG |
| | | CTCTAAATCGGGGGCTCCCTTTAGGGTTCCGATTTAGTGCTTTACGGCACC |
| | | TCGACCCCAAAAAACTTGATTAGGGTGATGGTTCACGTAGTGGGCCATCG |
| | | CCCTGATAGACGGTTTTTCGCCCTTTGACGTTGGAGTCCACGTTCTTTAAT |
| | | AGTGGACTCTTGTTCCAAACTGGAACAACACTCAACCCTATCTCGGTCTA |
| | | TTCTTTTGATTTATAAGGGATTTTGCCGATTTCGGCCTATTGGTTAAAAAA |
| | | TGAGCTGATTTAACAAAAATTTAACGCGAATTTTAACAAAATATTAACGT |
| | | TTACAATTTCCCAGGTGGCACTTTTCGGGGAAATGTGCGCGGAACCCCTA |
| | | TTTGTTTATTTTTCTAAATACATTCAAATATGTATCCGCTCATGAGACAAT |
| | | AACCCTGATAAATGCTTCAATAATATTGAAAAAGGAAGAGTATGAGTATT |
| | | CAACATTTCCGTGTCGCCCTTATTCCCTTTTTTGCGGCATTTTGCCTTCCTG |
| | | TTTTTGCTCACCCAGAAACGCTGGTGAAAGTAAAAGATGCTGAAGATCAG |
| | | TTGGGTGCACGAGTGGGTTACATCGAACTGGATCTCAACAGCGGTAAGAT |
| | | CCTTGAGAGTTTTCGCCCCGAAGAACGTTTTCCAATGATGAGCACTTTTA |
| | | AAGTTCTGCTATGTGGCGCGGTATTATCCCGTATTGACGCCGGGCAAGAG |
| | | CAACTCGGTCGCCGCATACACTATTCTCAGAATGACTTGGTTGAGTACTC |
| | | ACCAGTCACAGAAAAGCATCTTACGGATGGCATGACAGTAAGAGAATTA |
| | | TGCAGTGCTGCCATAACCATGAGTGATAACACTGCGGCCAACTTACTTCT |
| | | GACAACGATCGGAGGACCGAAGGAGCTAACCGCTTTTTTGCACAACATG |
| | | GGGGATCATGTAACTCGCCTTGATCGTTGGGAACCGGAGCTGAATGAAGC |
| | | CATACCAAACGACGAGCGTGACACCACGATGCCTGTAGCAATGGCAACA |
| | | ACGTTGCGCAAACTATTAACTGGCGAACTACTTACTCTAGCTTCCCGGCA |
| | | ACAATTAATAGACTGGATGGAGGCGGATAAAGTTGCAGGACCACTTCTG |
| | | CGCTCGGCCCTTCCGGCTGGCTGGTTTATTGCTGATAAATCTGGAGCCGG |
| | | TGAGCGTGGGTCTCGCGGTATCATTGCAGCACTGGGGCCAGATGGTAAGC |
| | | CCTCCCGTATCGTAGTTATCTACACGACGGGGAGTCAGGCAACTATGGAT |
| | | GAACGAAATAGACAGATCGCTGAGATAGGTGCCTCACTGATTAAGCATT |
| | | GGTAACTGTCAGACCAAGTTTACTCATATATACTTTAGATTGATTTAAAA |
| | | CTTCATTTTTAATTTAAAAGGATCTAGGTGAAGATCCTTTTTGATAATCTC |
| | | ATGACCAAAATCCCTTAACGTGAGTTTTCGTTCCACTGAGCGTCAGACCC |
| | | CGTAGAAAAGATCAAAGGATCTTCTTGAGATCCTTTTTTTCTGCGCGTAAT |
| | | CTGCTGCTTGCAAACAAAAAAACCACCGCTACCAGCGGTGGTTTGTTTGC |
| | | CGGATCAAGAGCTACCAACTCTTTTTCCGAAGGTAACTGGCTTCAGCAGA |
| | | GCGCAGATACCAAATACTGTCCTTCTAGTGTAGCCGTAGTTAGGCCACCA |
| | | CTTCAAGAACTCTGTAGCACCGCCTACATACCTCGCTCTGCTAATCCTGTT |
| | | ACCAGTGGCTGCTGCCAGTGGCGATAAGTCGTGTCTTACCGGGTTGGACT |
| | | CAAGACGATAGTTACCGGATAAGGCGCAGCGGTCGGGCTGAACGGGGGG |
| | | TTCGTGCACACAGCCCAGCTTGGAGCGAACGACCTACACCGAACTGAGAT |
| | | ACCTACAGCGTGAGCTATGAGAAAGCGCCACGCTTCCCGAAGGGAGAAA |
| | | GGCGGACAGGTATCCGGTAAGCGGCAGGGTCGGAACAGGAGAGCGCACG |
| | | AGGGAGCTTCCAGGGGGAAACGCCTGGTATCTTTATAGTCCTGTCGGGTT |
| | | TCGCCACCTCTGACTTGAGCGTCGATTTTTGTGATGCTCGTCAGGGGGGC |
| | | GGAGCCTATGGAAAAACGCCAGCAACGCGGCCTTTTTACGGTTCCTGGCC |
| | | TTTTGCTGGCCTTTTGCTCACATGTTCTTTCCTGCGTTATCCCCTGATTCTG |
| | | TGGATAACCGTATTACCGCCTTTGAGTGAGCTGATACCGCTCGCCGCAGC |
| | | CGAACGACCGAGCGCAGCGAGTCAGTGAGCGAGGAAGCGGAAGAGCGC |
| | | CCAATACGCAAACCGCCTCTCCCCGCGCGTTGGCCGATTCATTAATGCAG |
| | | CTGGCACGACAGGTTTCCCGACTGGAAAGCGGGCAGTGAGCGCAACGCA |
| | | ATTAATGTGAGTTAGCTCACTCATTAGGCACCCCAGGCTTTACACTTTATG |
| | | CTTCCGGCTCGTATGTTGTGTGGAATTGTGAGCGGATAACAATTTCACAC |
| | | AGGAAACAGCTATGACCATGATTACGCCAAGCGCGCAATTAACCCTCACT |
| | | AAAGGGAACAAAAGCTGGAGCTGCA |
| 66 | expression | AGCTTAATGTAGTCTTATGCAATACTCTTGTAGTCTTGCAACATGGTAACG |
| | vector | ATGAGTTAGCAACATGCCTTACAAGGAGAGAAAAAGCACCGTGCATGCC |
| | | GATTGGTGGAAGTAAGGTGGTACGATCGTGCCTTATTAGGAAGGCAACA |
| | | GACGGGTCTGACATGGATTGGACGAACCACTGAATTGCCGCATTGCAGA |
| | | GATATTGTATTTAAGTGCCTAGCTCGATACAATAAACGGGTCTCTCTGGTT |
| | | AGACCAGATCTGAGCCTGGGAGCTCTCTGGCTAACTAGGGAACCCACTGC |
| | | TTAAGCCTCAATAAAGCTTGCCTTGAGTGCTTCAAGTAGTGTGTGCCCGT |
| | | CTGTTGTGTGACTCTGGTAACTAGAGATCCCTCAGACCCTTTTAGTCAGTG |
| | | TGGAAAATCTCTAGCAGTGGCGCCCGAACAGGGACTTGAAAGCGAAAGG |
| | | GAAACCAGAGGAGCTCTCTCGACGCAGGACTCGGCTTGCTGAAGCGCGC |
| | | ACGGCAAGAGGCGAGGGGCGGCGACTGGTGAGTACGCCAAAAATTTTGA |
| | | CTAGCGGAGGCTAGAAGGAGAGAGATGGGTGCGAGAGCGTCAGTATTAA |
| | | GCGGGGGAGAATTAGATCGCGATGGGAAAAAATTCGGTTAAGGCCAGGG |
| | | GGAAAGAAAAAATATAAATTAAAACATATAGTATGGGCAAGCAGGGAGC |
| | | TAGAACGATTCGCAGTTAATCCTGGCCTGTTAGAAACATCAGAAGGCTGT |
| | | AGACAAATACTGGGACAGCTACAACCATCCCTTCAGACAGGATCAGAAG |
| | | AACTTAGATCATTATATAATACAGTAGCAACCCTCTATTGTGTGCATCAA |
| | | AGGATAGAGATAAAAGACACCAAGGAAGCTTTAGACAAGATAGAGGAA |
| | | GAGCAAAACAAAAGTAAGACCACCGCACAGCAAGCGGCCGCTGATCTTC |
| | | AGACCTGGAGGAGGAGATATGAGGGACAATTGGAGAAGTGAATTATATA |
| | | AATATAAAGTAGTAAAAATTGAACCATTAGGAGTAGCACCCACCAAGGC |
| | | AAAGAGAAGAGTGGTGCAGAGAGAAAAAAGAGCAGTGGGAATAGGAGC |
| | | TTTGTTCCTTGGGTTCTTGGGAGCAGCAGGAAGCACTATGGGCGCAGCCT |
| | | CAATGACGCTGACGGTACAGGCCAGACAATTATTGTCTGGTATAGTGCAG |
| | | CAGCAGAACAATTTGCTGAGGGCTATTGAGGCGCAACAGCATCTGTTGCA |
| | | ACTCACAGTCTGGGGCATCAAGCAGCTCCAGGCAAGAATCCTGGCTGTGG |
| | | AAAGATACCTAAAGGATCAACAGCTCCTGGGGATTTGGGGTTGCTCTGGA |
| | | AAACTCATTTGCACCACTGCTGTGCCTTGGAATGCTAGTTGGAGTAATAA |
| | | ATCTCTGGAACAGATTTGGAATCACACGACCTGGATGGAGTGGGACAGA |
| | | GAAATTAACAATTACACAAGCTTAATACACTCCTTAATTGAAGAATCGCA |
| | | AAACCAGCAAGAAAAGAATGAACAAGAATTATTGGAATTAGATAAATGG |
| | | GCAAGTTTGTGGAATTGGTTTAACATAACAAATTGGCTGTGGTATATAAA |
| | | ATTATTCATAATGATAGTAGGAGGCTTGGTAGGTTTAAGAATAGTTTTTG |
| | | CTGTACTTTCTATAGTGAATAGAGTTAGGCAGGGATATTCACCATTATCG |
| | | TTTCAGACCCACCTCCCAACCCCGAGGGGACCCGACAGGCCCGAAGGAA |
| | | TAGAAGAAGAAGGTGGAGAGAGAGACAGAGACAGATCCATTCGATTAGT |
| | | GAACGGATCTCGACGGTATCGGTTAACTTTTAAAAGAAAAGGGGGGATT |
| | | GGGGGGTACAGTGCAGGGGAAAGAATAGTAGACATAATAGCAACAGAC |
| | | ATACAAACTAAAGAATTACAAAAACAAATTACAAAAATTCAAAATTTTAT |
| | | CGATCACGAGACTAGCCTCGAGAAGCTTGATATCGAATTCCCACGGGGTT |
| | | GGACGCGTAGGAACAGAGAAACAGGAGAATATGGGCCAAACAGGATAT |
| | | CTGTGGTAAGCAGTTCCTGCCCCGGCTCAGGGCCAAGAACAGTTGGAACA |
| | | GCAGAATATGGGCCAAACAGGATATCTGTGGTAAGCAGTTCCTGCCCCGG |
| | | CTCAGGGCCAAGAACAGATGGTCCCCAGATGCGGTCCCGCCCTCAGCAGT |
| | | TTCTAGAGAACCATCAGATGTTTCCAGGGTGCCCCAAGGACCTGAAATGA |
| | | CCCTGTGCCTTATTTGAACTAACCAATCAGTTCGCTTCTCGCTTCTGTTCG |
| | | CGCGCTTCTGCTCCCCGAGCTCTATATAAGCAGAGCTCGTTTAGTGAACC |
| | | GTCAGATCGCTAGCACCGGTGCCGCCACCATGCCTCTGGGCCTGCTGTGG |
| | | CTGGGCCTGGCCCTGCTGGGCGCCCTGCACGCCCAGGCCGGCGTGCAGGT |
| | | GGAGACAATCTCCCCAGGCGACGGACGCACATTCCCTAAGCGGGGCCAG |
| | | ACCTGCGTGGTGCACTATACAGGCATGCTGGAGGATGGCAAGAAGTTTG |
| | | ACAGCTCCCGGGATAGAAACAAGCCATTCAAGTTTATGCTGGGCAAGCA |
| | | GGAAGTGATCAGAGGCTGGGAGGAGGGCGTGGCCCAGATGTCTGTGGGC |
| | | CAGAGGGCCAAGCTGACCATCAGCCCAGACTACGCCTATGGAGCAACAG |
| | | GCCACCCAGGAATCATCCCACCTCACGCCACCCTGGTGTTCGATGTGGAG |
| | | CTGCTGAAGCTGGGCGAGCAAAACTTGGTGATTCCTTGGGCCCCAGAAAA |
| | | TCTCACGCTTCACAAGTTGTCCGAATCCCAGCTCGAGCTCAACTGGAATA |
| | | ATAGATTTCTTAATCATTGTTTGGAACACCTGGTTCAATATAGAACGGATT |
| | | GGGACCACTCATGGACCGAGCAGTCAGTTGACTACCGCCACAAATTTTCA |
| | | CTTCCCAGCGTAGATGGGCAGAAGAGGTACACATTTAGGGTCAGATCCA |
| | | GGTTTAATCCTCTGTGTGGTTCTGCTCAACACTGGTCTGAGTGGAGCCATC |
| | | CGATCCACTGGGGCTCAAATACCTCTAAAGAAAATCCGTTCCTCTTTGCG |
| | | CTCGAAGCCGTTGTTATCAGCGTCGGAAGCATGGGACTTATCATTTCCCTT |
| | | CTCTGCGTGTACTTCTGGCTGGAGCGGACGATGCCGCGGATTCCGACGCT |
| | | CAAAAACCTGGAGGACCTTGTAACAGAATATCACGGTAATTTCTCCGCTT |
| | | GGAGTGGCGTATCAAAGGGGCTTGCTGAGTCCCTTCAACCGGATTACTCT |
| | | GAGCGCCTCTGCTTGGTGTCCGAGATACCTCCCAAAGGAGGTGCACTTGG |
| | | GGAGGGGCCAGGCGCGTCCCCTTGCAATCAGCATAGTCCGTATTGGGCGC |
| | | CCCCCTGTTATACCCTCAAACCGGAAACGGGAAGCGGAGCTACTAACTTC |
| | | AGCCTGCTGAAGCAGGCTGGAGACGTGGAGGAGAACCCTGGACCTATGG |
| | | CACTGCCCGTGACCGCCCTGCTGCTGCCTCTGGCCCTGCTGCTGCACGCA |
| | | GCCCGGCCTATCCTGTGGCACGAGATGTGGCACGAGGGCCTGGAGGAGG |
| | | CCAGCAGGCTGTATTTTGGCGAGCGCAACGTGAAGGGCATGTTCGAGGTG |
| | | CTGGAGCCTCTGCACGCCATGATGGAGAGAGGCCCACAGACCCTGAAGG |
| | | AGACATCCTTTAACCAGGCCTATGGACGGGACCTGATGGAGGCACAGGA |
| | | GTGGTGCAGAAAGTACATGAAGTCTGGCAATGTGAAGGACCTGCTGCAG |
| | | GCCTGGGATCTGTACTATCACGTGTTTCGGAGAATCTCCAAGAAACCTTT |
| | | TGAGAACCTTAGACTGATGGCGCCCATCTCTCTGCAGGTAGTTCACGTTG |
| | | AGACCCATAGATGCAATATAAGCTGGGAAATCTCACAAGCCAGCCATTA |
| | | CTTTGAACGGCATTTGGAATTCGAGGCCCGAACACTTTCCCCCGGTCATA |
| | | CGTGGGAAGAAGCTCCTCTCTTGACGCTGAAGCAGAAGCAGGAGTGGAT |
| | | TTGTCTGGAGACTTTGACTCCTGATACTCAGTATGAGTTCCAAGTTCGGGT |
| | | GAAACCACTCCAAGGCGAGTTCACGACGTGGTCTCCGTGGAGTCAACCGT |
| | | TGGCGTTCCGCACGAAGCCCGCTGCCCTTGGCAAAGACACGATTCCGTGG |
| | | CTTGGGCATCTGCTCGTTGGGCTGAGTGGTGCGTTTGGTTTCATCATCTTG |
| | | GTCTATCTCTTGATCAATTGCAGAAATACAGGCCCTTGGCTGAAAAAAGT |
| | | GCTCAAGTGTAATACCCCCGACCCAAGCAAGTTCTTCTCCCAGCTTTCTTC |
| | | AGAGCATGGAGGCGATGTGCAGAAATGGCTCTCTTCACCTTTTCCCTCCT |
| | | CAAGCTTCTCCCCGGGAGGGCTGGCGCCCGAGATTTCACCTCTTGAGGTA |
| | | CTTGAACGAGACAAGGTTACCCAACTTCTCCTTCAACAGGATAAGGTACC |
| | | CGAACCTGCGAGCCTTAGCTCCAACCACTCTCTTACGAGCTGCTTCACCA |
| | | ATCAGGGATACTTCTTTTTCCACCTTCCCGATGCGCTGGAAATCGAAGCTT |
| | | GTCAAGTTTACTTTACCTATGATCCATATAGCGAGGAAGATCCCGACGAA |
| | | GGAGTCGCCGGTGCGCCCACGGGTTCCTCACCCCAACCTCTCCAGCCTCT |
| | | CTCAGGAGAAGATGATGCTTATTGCACTTTTCCCAGTAGAGACGATCTCC |
| | | TCCTCTTTTCTCCATCTCTTTTGGGGGGACCTTCCCCCCCTTCTACGGCACC |
| | | TGGCGGGTCTGGTGCTGGCGAGGAGCGGATGCCGCCGTCCCTCCAGGAG |
| | | CGAGTACCACGAGATTGGGATCCCCAGCCACTTGGACCCCCCACCCCCGG |
| | | CGTACCTGACCTTGTCGATTTTCAACCTCCCCCTGAATTGGTGCTGCGAGA |
| | | GGCTGGGGAGGAAGTTCCGGACGCTGGGCCGAGGGAGGGCGTGTCCTTT |
| | | CCATGGAGTAGGCCTCCAGGTCAAGGCGAGTTTAGGGCTCTCAACGCGCG |
| | | GCTGCCGTTGAATACAGACGCTTATCTCTCACTGCAGGAACTGCAAGGTC |
| | | AGGACCCAACACATCTTGTAGGATCTGGTGCTACTAATTTTTCTCTTTTGA |
| | | AGCAAGCTGGAGATGTTGAAGAGAACCCTGGTCCAGTGAGCAAGGGCGA |
| | | GGAGCTGTTCACCGGGGTGGTGCCCATCCTGGTCGAGCTGGACGGCGACG |
| | | TAAACGGCCACAAGTTCAGCGTGTCCGGCGAGGGCGAGGGCGATGCCAC |
| | | CTACGGCAAGCTGACCCTGAAGTTCATCTGCACCACCGGCAAGCTGCCCG |
| | | TGCCCTGGCCCACCCTCGTGACCACCCTGACCTACGGCGTGCAGTGCTTC |
| | | AGCCGCTACCCCGACCACATGAAGCAGCACGACTTCTTCAAGTCCGCCAT |
| | | GCCCGAAGGCTACGTCCAGGAGCGCACCATCTTCTTCAAGGACGACGGC |
| | | AACTACAAGACCCGCGCCGAGGTGAAGTTCGAGGGCGACACCCTGGTGA |
| | | ACCGCATCGAGCTGAAGGGCATCGACTTCAAGGAGGACGGCAACATCCT |
| | | GGGGCACAAGCTGGAGTACAACTACAACAGCCACAACGTCTATATCATG |
| | | GCCGACAAGCAGAAGAACGGCATCAAGGTGAACTTCAAGATCCGCCACA |
| | | ACATCGAGGACGGCAGCGTGCAGCTCGCCGACCACTACCAGCAGAACAC |
| | | CCCCATCGGCGACGGCCCCGTGCTGCTGCCCGACAACCACTACCTGAGCA |
| | | CCCAGTCCGCCCTGAGCAAAGACCCCAACGAGAAGCGCGATCACATGGT |
| | | CCTGCTGGAGTTCGTGACCGCCGCCGGGATCACTCTCGGCATGGACGAGC |
| | | TGTACAAGTAAACTAGTGTCGACAATCAACCTCTGGATTACAAAATTTGT |
| | | GAAAGATTGACTGGTATTCTTAACTATGTTGCTCCTTTTACGCTATGTGGA |
| | | TACGCTGCTTTAATGCCTTTGTATCATGCTATTGCTTCCCGTATGGCTTTC |
| | | ATTTTCTCCTCCTTGTATAAATCCTGGTTGCTGTCTCTTTATGAGGAGTTGT |
| | | GGCCCGTTGTCAGGCAACGTGGCGTGGTGTGCACTGTGTTTGCTGACGCA |
| | | ACCCCCACTGGTTGGGGCATTGCCACCACCTGTCAGCTCCTTTCCGGGAC |
| | | TTTCGCTTTCCCCCTCCCTATTGCCACGGCGGAACTCATCGCCGCCTGCCT |
| | | TGCCCGCTGCTGGACAGGGGCTCGGCTGTTGGGCACTGACAATTCCGTGG |
| | | TGTTGTCGGGGAAGCTGACGTCCTTTCCATGGCTGCTCGCCTGTGTTGCCA |
| | | CCTGGATTCTGCGCGGGACGTCCTTCTGCTACGTCCCTTCGGCCCTCAATC |
| | | CAGCGGACCTTCCTTCCCGCGGCCTGCTGCCGGCTCTGCGGCCTCTTCCGC |
| | | GTCTTCGCCTTCGCCCTCAGACGAGTCGGATCTCCCTTTGGGCCGCCTCCC |
| | | CGCCTGGAATTCGAGCTCGGTACCTTTAAGACCAATGACTTACAAGGCAG |
| | | CTGTAGATCTTAGCCACTTTTTAAAAGAAAAGGGGGGACTGGAAGGGCT |
| | | AATTCACTCCCAACGAAGACAAGATCTGCTTTTTGCTTGTACTGGGTCTCT |
| | | CTGGTTAGACCAGATCTGAGCCTGGGAGCTCTCTGGCTAACTAGGGAACC |
| | | CACTGCTTAAGCCTCAATAAAGCTTGCCTTGAGTGCTTCAAGTAGTGTGT |
| | | GCCCGTCTGTTGTGTGACTCTGGTAACTAGAGATCCCTCAGACCCTTTTAG |
| | | TCAGTGTGGAAAATCTCTAGCAGTAGTAGTTCATGTCATCTTATTATTCAG |
| | | TATTTATAACTTGCAAAGAAATGAATATCAGAGAGTGAGAGGAACTTGTT |
| | | TATTGCAGCTTATAATGGTTACAAATAAAGCAATAGCATCACAAATTTCA |
| | | CAAATAAAGCATTTTTTTCACTGCATTCTAGTTGTGGTTTGTCCAAACTCA |
| | | TCAATGTATCTTATCATGTCTGGCTCTAGCTATCCCGCCCCTAACTCCGCC |
| | | CAGTTCCGCCCATTCTCCGCCCCATGGCTGACTAATTTTTTTTATTTATGC |
| | | AGAGGCCGAGGCCGCCTCGGCCTCTGAGCTATTCCAGAAGTAGTGAGGA |
| | | GGCTTTTTTGGAGGCCTAGGCTTTTGCGTCGAGACGTACCCAATTCGCCCT |
| | | ATAGTGAGTCGTATTACGCGCGCTCACTGGCCGTCGTTTTACAACGTCGT |
| | | GACTGGGAAAACCCTGGCGTTACCCAACTTAATCGCCTTGCAGCACATCC |
| | | CCCTTTCGCCAGCTGGCGTAATAGCGAAGAGGCCCGCACCGATCGCCCTT |
| | | CCCAACAGTTGCGCAGCCTGAATGGCGAATGGCGCGACGCGCCCTGTAG |
| | | CGGCGCATTAAGCGCGGCGGGTGTGGTGGTTACGCGCAGCGTGACCGCT |
| | | ACACTTGCCAGCGCCCTAGCGCCCGCTCCTTTCGCTTTCTTCCCTTCCTTTC |
| | | TCGCCACGTTCGCCGGCTTTCCCCGTCAAGCTCTAAATCGGGGGCTCCCTT |
| | | TAGGGTTCCGATTTAGTGCTTTACGGCACCTCGACCCCAAAAAACTTGAT |
| | | TAGGGTGATGGTTCACGTAGTGGGCCATCGCCCTGATAGACGGTTTTTCG |
| | | CCCTTTGACGTTGGAGTCCACGTTCTTTAATAGTGGACTCTTGTTCCAAAC |
| | | TGGAACAACACTCAACCCTATCTCGGTCTATTCTTTTGATTTATAAGGGAT |
| | | TTTGCCGATTTCGGCCTATTGGTTAAAAAATGAGCTGATTTAACAAAAAT |
| | | TTAACGCGAATTTTAACAAAATATTAACGTTTACAATTTCCCAGGTGGCA |
| | | CTTTTCGGGGAAATGTGCGCGGAACCCCTATTTGTTTATTTTTCTAAATAC |
| | | ATTCAAATATGTATCCGCTCATGAGACAATAACCCTGATAAATGCTTCAA |
| | | TAATATTGAAAAAGGAAGAGTATGAGTATTCAACATTTCCGTGTCGCCCT |
| | | TATTCCCTTTTTTGCGGCATTTTGCCTTCCTGTTTTTGCTCACCCAGAAACG |
| | | CTGGTGAAAGTAAAAGATGCTGAAGATCAGTTGGGTGCACGAGTGGGTT |
| | | ACATCGAACTGGATCTCAACAGCGGTAAGATCCTTGAGAGTTTTCGCCCC |
| | | GAAGAACGTTTTCCAATGATGAGCACTTTTAAAGTTCTGCTATGTGGCGC |
| | | GGTATTATCCCGTATTGACGCCGGGCAAGAGCAACTCGGTCGCCGCATAC |
| | | ACTATTCTCAGAATGACTTGGTTGAGTACTCACCAGTCACAGAAAAGCAT |
| | | CTTACGGATGGCATGACAGTAAGAGAATTATGCAGTGCTGCCATAACCAT |
| | | GAGTGATAACACTGCGGCCAACTTACTTCTGACAACGATCGGAGGACCG |
| | | AAGGAGCTAACCGCTTTTTTGCACAACATGGGGGATCATGTAACTCGCCT |
| | | TGATCGTTGGGAACCGGAGCTGAATGAAGCCATACCAAACGACGAGCGT |
| | | GACACCACGATGCCTGTAGCAATGGCAACAACGTTGCGCAAACTATTAAC |
| | | TGGCGAACTACTTACTCTAGCTTCCCGGCAACAATTAATAGACTGGATGG |
| | | AGGCGGATAAAGTTGCAGGACCACTTCTGCGCTCGGCCCTTCCGGCTGGC |
| | | TGGTTTATTGCTGATAAATCTGGAGCCGGTGAGCGTGGGTCTCGCGGTAT |
| | | CATTGCAGCACTGGGGCCAGATGGTAAGCCCTCCCGTATCGTAGTTATCT |
| | | ACACGACGGGGAGTCAGGCAACTATGGATGAACGAAATAGACAGATCGC |
| | | TGAGATAGGTGCCTCACTGATTAAGCATTGGTAACTGTCAGACCAAGTTT |
| | | ACTCATATATACTTTAGATTGATTTAAAACTTCATTTTTAATTTAAAAGGA |
| | | TCTAGGTGAAGATCCTTTTTGATAATCTCATGACCAAAATCCCTTAACGTG |
| | | AGTTTTCGTTCCACTGAGCGTCAGACCCCGTAGAAAAGATCAAAGGATCT |
| | | TCTTGAGATCCTTTTTTTCTGCGCGTAATCTGCTGCTTGCAAACAAAAAAA |
| | | CCACCGCTACCAGCGGTGGTTTGTTTGCCGGATCAAGAGCTACCAACTCT |
| | | TTTTCCGAAGGTAACTGGCTTCAGCAGAGCGCAGATACCAAATACTGTCC |
| | | TTCTAGTGTAGCCGTAGTTAGGCCACCACTTCAAGAACTCTGTAGCACCG |
| | | CCTACATACCTCGCTCTGCTAATCCTGTTACCAGTGGCTGCTGCCAGTGGC |
| | | GATAAGTCGTGTCTTACCGGGTTGGACTCAAGACGATAGTTACCGGATAA |
| | | GGCGCAGCGGTCGGGCTGAACGGGGGGTTCGTGCACACAGCCCAGCTTG |
| | | GAGCGAACGACCTACACCGAACTGAGATACCTACAGCGTGAGCTATGAG |
| | | AAAGCGCCACGCTTCCCGAAGGGAGAAAGGCGGACAGGTATCCGGTAAG |
| | | CGGCAGGGTCGGAACAGGAGAGCGCACGAGGGAGCTTCCAGGGGGAAA |
| | | CGCCTGGTATCTTTATAGTCCTGTCGGGTTTCGCCACCTCTGACTTGAGCG |
| | | TCGATTTTTGTGATGCTCGTCAGGGGGGCGGAGCCTATGGAAAAACGCCA |
| | | GCAACGCGGCCTTTTTACGGTTCCTGGCCTTTTGCTGGCCTTTTGCTCACA |
| | | TGTTCTTTCCTGCGTTATCCCCTGATTCTGTGGATAACCGTATTACCGCCT |
| | | TTGAGTGAGCTGATACCGCTCGCCGCAGCCGAACGACCGAGCGCAGCGA |
| | | GTCAGTGAGCGAGGAAGCGGAAGAGCGCCCAATACGCAAACCGCCTCTC |
| | | CCCGCGCGTTGGCCGATTCATTAATGCAGCTGGCACGACAGGTTTCCCGA |
| | | CTGGAAAGCGGGCAGTGAGCGCAACGCAATTAATGTGAGTTAGCTCACT |
| | | CATTAGGCACCCCAGGCTTTACACTTTATGCTTCCGGCTCGTATGTTGTGT |
| | | GGAATTGTGAGCGGATAACAATTTCACACAGGAAACAGCTATGACCATG |
| | | ATTACGCCAAGCGCGCAATTAACCCTCACTAAAGGGAACAAAAGCTGGA |
| | | GCTGCA |
| 67 | expression | AGCTTAATGTAGTCTTATGCAATACTCTTGTAGTCTTGCAACATGGTAACG |
| | vector | ATGAGTTAGCAACATGCCTTACAAGGAGAGAAAAAGCACCGTGCATGCC |
| | | GATTGGTGGAAGTAAGGTGGTACGATCGTGCCTTATTAGGAAGGCAACA |
| | | GACGGGTCTGACATGGATTGGACGAACCACTGAATTGCCGCATTGCAGA |
| | | GATATTGTATTTAAGTGCCTAGCTCGATACAATAAACGGGTCTCTCTGGTT |
| | | AGACCAGATCTGAGCCTGGGAGCTCTCTGGCTAACTAGGGAACCCACTGC |
| | | TTAAGCCTCAATAAAGCTTGCCTTGAGTGCTTCAAGTAGTGTGTGCCCGT |
| | | CTGTTGTGTGACTCTGGTAACTAGAGATCCCTCAGACCCTTTTAGTCAGTG |
| | | TGGAAAATCTCTAGCAGTGGCGCCCGAACAGGGACTTGAAAGCGAAAGG |
| | | GAAACCAGAGGAGCTCTCTCGACGCAGGACTCGGCTTGCTGAAGCGCGC |
| | | ACGGCAAGAGGCGAGGGGCGGCGACTGGTGAGTACGCCAAAAATTTTGA |
| | | CTAGCGGAGGCTAGAAGGAGAGAGATGGGTGCGAGAGCGTCAGTATTAA |
| | | GCGGGGGAGAATTAGATCGCGATGGGAAAAAATTCGGTTAAGGCCAGGG |
| | | GGAAAGAAAAAATATAAATTAAAACATATAGTATGGGCAAGCAGGGAGC |
| | | TAGAACGATTCGCAGTTAATCCTGGCCTGTTAGAAACATCAGAAGGCTGT |
| | | AGACAAATACTGGGACAGCTACAACCATCCCTTCAGACAGGATCAGAAG |
| | | AACTTAGATCATTATATAATACAGTAGCAACCCTCTATTGTGTGCATCAA |
| | | AGGATAGAGATAAAAGACACCAAGGAAGCTTTAGACAAGATAGAGGAA |
| | | GAGCAAAACAAAAGTAAGACCACCGCACAGCAAGCGGCCGCTGATCTTC |
| | | AGACCTGGAGGAGGAGATATGAGGGACAATTGGAGAAGTGAATTATATA |
| | | AATATAAAGTAGTAAAAATTGAACCATTAGGAGTAGCACCCACCAAGGC |
| | | AAAGAGAAGAGTGGTGCAGAGAGAAAAAAGAGCAGTGGGAATAGGAGC |
| | | TTTGTTCCTTGGGTTCTTGGGAGCAGCAGGAAGCACTATGGGCGCAGCCT |
| | | CAATGACGCTGACGGTACAGGCCAGACAATTATTGTCTGGTATAGTGCAG |
| | | CAGCAGAACAATTTGCTGAGGGCTATTGAGGCGCAACAGCATCTGTTGCA |
| | | ACTCACAGTCTGGGGCATCAAGCAGCTCCAGGCAAGAATCCTGGCTGTGG |
| | | AAAGATACCTAAAGGATCAACAGCTCCTGGGGATTTGGGGTTGCTCTGGA |
| | | AAACTCATTTGCACCACTGCTGTGCCTTGGAATGCTAGTTGGAGTAATAA |
| | | ATCTCTGGAACAGATTTGGAATCACACGACCTGGATGGAGTGGGACAGA |
| | | GAAATTAACAATTACACAAGCTTAATACACTCCTTAATTGAAGAATCGCA |
| | | AAACCAGCAAGAAAAGAATGAACAAGAATTATTGGAATTAGATAAATGG |
| | | GCAAGTTTGTGGAATTGGTTTAACATAACAAATTGGCTGTGGTATATAAA |
| | | ATTATTCATAATGATAGTAGGAGGCTTGGTAGGTTTAAGAATAGTTTTTG |
| | | CTGTACTTTCTATAGTGAATAGAGTTAGGCAGGGATATTCACCATTATCG |
| | | TTTCAGACCCACCTCCCAACCCCGAGGGGACCCGACAGGCCCGAAGGAA |
| | | TAGAAGAAGAAGGTGGAGAGAGAGACAGAGACAGATCCATTCGATTAGT |
| | | GAACGGATCTCGACGGTATCGGTTAACTTTTAAAAGAAAAGGGGGGATT |
| | | GGGGGGTACAGTGCAGGGGAAAGAATAGTAGACATAATAGCAACAGAC |
| | | ATACAAACTAAAGAATTACAAAAACAAATTACAAAAATTCAAAATTTTAT |
| | | CGATCACGAGACTAGCCTCGAGAAGCTTGATATCGAATTCCCACGGGGTT |
| | | GGACGCGTAGGAACAGAGAAACAGGAGAATATGGGCCAAACAGGATAT |
| | | CTGTGGTAAGCAGTTCCTGCCCCGGCTCAGGGCCAAGAACAGTTGGAACA |
| | | GCAGAATATGGGCCAAACAGGATATCTGTGGTAAGCAGTTCCTGCCCCGG |
| | | CTCAGGGCCAAGAACAGATGGTCCCCAGATGCGGTCCCGCCCTCAGCAGT |
| | | TTCTAGAGAACCATCAGATGTTTCCAGGGTGCCCCAAGGACCTGAAATGA |
| | | CCCTGTGCCTTATTTGAACTAACCAATCAGTTCGCTTCTCGCTTCTGTTCG |
| | | CGCGCTTCTGCTCCCCGAGCTCTATATAAGCAGAGCTCGTTTAGTGAACC |
| | | GTCAGATCGCTAGCACCGGTGCCGCCACCATGCCTCTGGGCCTGCTGTGG |
| | | CTGGGCCTGGCCCTGCTGGGCGCCCTGCACGCCCAGGCCGGCGTGCAGGT |
| | | GGAGACAATCTCCCCAGGCGACGGACGCACATTCCCTAAGCGGGGCCAG |
| | | ACCTGCGTGGTGCACTATACAGGCATGCTGGAGGATGGCAAGAAGTTTG |
| | | ACAGCTCCCGGGATAGAAACAAGCCATTCAAGTTTATGCTGGGCAAGCA |
| | | GGAAGTGATCAGAGGCTGGGAGGAGGGCGTGGCCCAGATGTCTGTGGGC |
| | | CAGAGGGCCAAGCTGACCATCAGCCCAGACTACGCCTATGGAGCAACAG |
| | | GCCACCCAGGAATCATCCCACCTCACGCCACCCTGGTGTTCGATGTGGAG |
| | | CTGCTGAAGCTGGGCGAGGGATCCAACACATCAAAAGAGAACCCCTTTCT |
| | | GTTCGCATTGGAGGCCGTAGTCATATCTGTTGGATCCATGGGACTTATTAT |
| | | CTCCCTGTTGTGTGTGTACTTCTGGCTGGAACGGACTATGCCCAGGATCCC |
| | | CACGCTCAAGAATCTGGAAGATCTCGTCACAGAATACCATGGTAATTTCA |
| | | GCGCCTGGAGCGGAGTCTCTAAGGGTCTGGCCGAATCCCTCCAACCCGAT |
| | | TATTCTGAACGGTTGTGCCTCGTATCCGAAATACCACCAAAAGGCGGGGC |
| | | TCTGGGTGAGGGCCCAGGGGCGAGTCCGTGCAATCAACACAGCCCGTATT |
| | | GGGCCCCTCCTTGTTATACGTTGAAGCCCGAAACTGGAAGCGGAGCTACT |
| | | AACTTCAGCCTGCTGAAGCAGGCTGGAGACGTGGAGGAGAACCCTGGAC |
| | | CTATGGCACTGCCCGTGACCGCCCTGCTGCTGCCTCTGGCCCTGCTGCTGC |
| | | ACGCAGCCCGGCCTATCCTGTGGCACGAGATGTGGCACGAGGGCCTGGA |
| | | GGAGGCCAGCAGGCTGTATTTTGGCGAGCGCAACGTGAAGGGCATGTTC |
| | | GAGGTGCTGGAGCCTCTGCACGCCATGATGGAGAGAGGCCCACAGACCC |
| | | TGAAGGAGACATCCTTTAACCAGGCCTATGGACGGGACCTGATGGAGGC |
| | | ACAGGAGTGGTGCAGAAAGTACATGAAGTCTGGCAATGTGAAGGACCTG |
| | | CTGCAGGCCTGGGATCTGTACTATCACGTGTTTCGGAGAATCTCCAAGGG |
| | | CAAAGACACGATTCCGTGGCTTGGGCATCTGCTCGTTGGGCTGAGTGGTG |
| | | CGTTTGGTTTCATCATCTTGGTCTATCTCTTGATCAATTGCAGAAATACAG |
| | | GCCCTTGGCTGAAAAAAGTGCTCAAGTGTAATACCCCCGACCCAAGCAA |
| | | GTTCTTCTCCCAGCTTTCTTCAGAGCATGGAGGCGATGTGCAGAAATGGC |
| | | TCTCTTCACCTTTTCCCTCCTCAAGCTTCTCCCCGGGAGGGCTGGCGCCCG |
| | | AGATTTCACCTCTTGAGGTACTTGAACGAGACAAGGTTACCCAACTTCTC |
| | | CTTCAACAGGATAAGGTACCCGAACCTGCGAGCCTTAGCTCCAACCACTC |
| | | TCTTACGAGCTGCTTCACCAATCAGGGATACTTCTTTTTCCACCTTCCCGA |
| | | TGCGCTGGAAATCGAAGCTTGTCAAGTTTACTTTACCTATGATCCATATA |
| | | GCGAGGAAGATCCCGACGAAGGAGTCGCCGGTGCGCCCACGGGTTCCTC |
| | | ACCCCAACCTCTCCAGCCTCTCTCAGGAGAAGATGATGCTTATTGCACTTT |
| | | TCCCAGTAGAGACGATCTCCTCCTCTTTTCTCCATCTCTTTTGGGGGGACC |
| | | TTCCCCCCCTTCTACGGCACCTGGCGGGTCTGGTGCTGGCGAGGAGCGGA |
| | | TGCCGCCGTCCCTCCAGGAGCGAGTACCACGAGATTGGGATCCCCAGCCA |
| | | CTTGGACCCCCCACCCCCGGCGTACCTGACCTTGTCGATTTTCAACCTCCC |
| | | CCTGAATTGGTGCTGCGAGAGGCTGGGGAGGAAGTTCCGGACGCTGGGC |
| | | CGAGGGAGGGCGTGTCCTTTCCATGGAGTAGGCCTCCAGGTCAAGGCGA |
| | | GTTTAGGGCTCTCAACGCGCGGCTGCCGTTGAATACAGACGCTTATCTCT |
| | | CACTGCAGGAACTGCAAGGTCAGGACCCAACACATCTTGTAGGATCTGGT |
| | | GCTACTAATTTTTCTCTTTTGAAGCAAGCTGGAGATGTTGAAGAGAACCC |
| | | TGGTCCAGTGAGCAAGGGCGAGGAGCTGTTCACCGGGGTGGTGCCCATC |
| | | CTGGTCGAGCTGGACGGCGACGTAAACGGCCACAAGTTCAGCGTGTCCG |
| | | GCGAGGGCGAGGGCGATGCCACCTACGGCAAGCTGACCCTGAAGTTCAT |
| | | CTGCACCACCGGCAAGCTGCCCGTGCCCTGGCCCACCCTCGTGACCACCC |
| | | TGACCTACGGCGTGCAGTGCTTCAGCCGCTACCCCGACCACATGAAGCAG |
| | | CACGACTTCTTCAAGTCCGCCATGCCCGAAGGCTACGTCCAGGAGCGCAC |
| | | CATCTTCTTCAAGGACGACGGCAACTACAAGACCCGCGCCGAGGTGAAG |
| | | TTCGAGGGCGACACCCTGGTGAACCGCATCGAGCTGAAGGGCATCGACTT |
| | | CAAGGAGGACGGCAACATCCTGGGGCACAAGCTGGAGTACAACTACAAC |
| | | AGCCACAACGTCTATATCATGGCCGACAAGCAGAAGAACGGCATCAAGG |
| | | TGAACTTCAAGATCCGCCACAACATCGAGGACGGCAGCGTGCAGCTCGC |
| | | CGACCACTACCAGCAGAACACCCCCATCGGCGACGGCCCCGTGCTGCTGC |
| | | CCGACAACCACTACCTGAGCACCCAGTCCGCCCTGAGCAAAGACCCCAA |
| | | CGAGAAGCGCGATCACATGGTCCTGCTGGAGTTCGTGACCGCCGCCGGG |
| | | ATCACTCTCGGCATGGACGAGCTGTACAAGTAAACTAGTGTCGACAATCA |
| | | ACCTCTGGATTACAAAATTTGTGAAAGATTGACTGGTATTCTTAACTATGT |
| | | TGCTCCTTTTACGCTATGTGGATACGCTGCTTTAATGCCTTTGTATCATGC |
| | | TATTGCTTCCCGTATGGCTTTCATTTTCTCCTCCTTGTATAAATCCTGGTTG |
| | | CTGTCTCTTTATGAGGAGTTGTGGCCCGTTGTCAGGCAACGTGGCGTGGT |
| | | GTGCACTGTGTTTGCTGACGCAACCCCCACTGGTTGGGGCATTGCCACCA |
| | | CCTGTCAGCTCCTTTCCGGGACTTTCGCTTTCCCCCTCCCTATTGCCACGG |
| | | CGGAACTCATCGCCGCCTGCCTTGCCCGCTGCTGGACAGGGGCTCGGCTG |
| | | TTGGGCACTGACAATTCCGTGGTGTTGTCGGGGAAGCTGACGTCCTTTCC |
| | | ATGGCTGCTCGCCTGTGTTGCCACCTGGATTCTGCGCGGGACGTCCTTCTG |
| | | CTACGTCCCTTCGGCCCTCAATCCAGCGGACCTTCCTTCCCGCGGCCTGCT |
| | | GCCGGCTCTGCGGCCTCTTCCGCGTCTTCGCCTTCGCCCTCAGACGAGTCG |
| | | GATCTCCCTTTGGGCCGCCTCCCCGCCTGGAATTCGAGCTCGGTACCTTTA |
| | | AGACCAATGACTTACAAGGCAGCTGTAGATCTTAGCCACTTTTTAAAAGA |
| | | AAAGGGGGGACTGGAAGGGCTAATTCACTCCCAACGAAGACAAGATCTG |
| | | CTTTTTGCTTGTACTGGGTCTCTCTGGTTAGACCAGATCTGAGCCTGGGAG |
| | | CTCTCTGGCTAACTAGGGAACCCACTGCTTAAGCCTCAATAAAGCTTGCC |
| | | TTGAGTGCTTCAAGTAGTGTGTGCCCGTCTGTTGTGTGACTCTGGTAACTA |
| | | GAGATCCCTCAGACCCTTTTAGTCAGTGTGGAAAATCTCTAGCAGTAGTA |
| | | GTTCATGTCATCTTATTATTCAGTATTTATAACTTGCAAAGAAATGAATAT |
| | | CAGAGAGTGAGAGGAACTTGTTTATTGCAGCTTATAATGGTTACAAATAA |
| | | AGCAATAGCATCACAAATTTCACAAATAAAGCATTTTTTTCACTGCATTCT |
| | | AGTTGTGGTTTGTCCAAACTCATCAATGTATCTTATCATGTCTGGCTCTAG |
| | | CTATCCCGCCCCTAACTCCGCCCAGTTCCGCCCATTCTCCGCCCCATGGCT |
| | | GACTAATTTTTTTTATTTATGCAGAGGCCGAGGCCGCCTCGGCCTCTGAGC |
| | | TATTCCAGAAGTAGTGAGGAGGCTTTTTTGGAGGCCTAGGCTTTTGCGTC |
| | | GAGACGTACCCAATTCGCCCTATAGTGAGTCGTATTACGCGCGCTCACTG |
| | | GCCGTCGTTTTACAACGTCGTGACTGGGAAAACCCTGGCGTTACCCAACT |
| | | TAATCGCCTTGCAGCACATCCCCCTTTCGCCAGCTGGCGTAATAGCGAAG |
| | | AGGCCCGCACCGATCGCCCTTCCCAACAGTTGCGCAGCCTGAATGGCGAA |
| | | TGGCGCGACGCGCCCTGTAGCGGCGCATTAAGCGCGGCGGGTGTGGTGG |
| | | TTACGCGCAGCGTGACCGCTACACTTGCCAGCGCCCTAGCGCCCGCTCCT |
| | | TTCGCTTTCTTCCCTTCCTTTCTCGCCACGTTCGCCGGCTTTCCCCGTCAAG |
| | | CTCTAAATCGGGGGCTCCCTTTAGGGTTCCGATTTAGTGCTTTACGGCACC |
| | | TCGACCCCAAAAAACTTGATTAGGGTGATGGTTCACGTAGTGGGCCATCG |
| | | CCCTGATAGACGGTTTTTCGCCCTTTGACGTTGGAGTCCACGTTCTTTAAT |
| | | AGTGGACTCTTGTTCCAAACTGGAACAACACTCAACCCTATCTCGGTCTA |
| | | TTCTTTTGATTTATAAGGGATTTTGCCGATTTCGGCCTATTGGTTAAAAAA |
| | | TGAGCTGATTTAACAAAAATTTAACGCGAATTTTAACAAAATATTAACGT |
| | | TTACAATTTCCCAGGTGGCACTTTTCGGGGAAATGTGCGCGGAACCCCTA |
| | | TTTGTTTATTTTTCTAAATACATTCAAATATGTATCCGCTCATGAGACAAT |
| | | AACCCTGATAAATGCTTCAATAATATTGAAAAAGGAAGAGTATGAGTATT |
| | | CAACATTTCCGTGTCGCCCTTATTCCCTTTTTTGCGGCATTTTGCCTTCCTG |
| | | TTTTTGCTCACCCAGAAACGCTGGTGAAAGTAAAAGATGCTGAAGATCAG |
| | | TTGGGTGCACGAGTGGGTTACATCGAACTGGATCTCAACAGCGGTAAGAT |
| | | CCTTGAGAGTTTTCGCCCCGAAGAACGTTTTCCAATGATGAGCACTTTTA |
| | | AAGTTCTGCTATGTGGCGCGGTATTATCCCGTATTGACGCCGGGCAAGAG |
| | | CAACTCGGTCGCCGCATACACTATTCTCAGAATGACTTGGTTGAGTACTC |
| | | ACCAGTCACAGAAAAGCATCTTACGGATGGCATGACAGTAAGAGAATTA |
| | | TGCAGTGCTGCCATAACCATGAGTGATAACACTGCGGCCAACTTACTTCT |
| | | GACAACGATCGGAGGACCGAAGGAGCTAACCGCTTTTTTGCACAACATG |
| | | GGGGATCATGTAACTCGCCTTGATCGTTGGGAACCGGAGCTGAATGAAGC |
| | | CATACCAAACGACGAGCGTGACACCACGATGCCTGTAGCAATGGCAACA |
| | | ACGTTGCGCAAACTATTAACTGGCGAACTACTTACTCTAGCTTCCCGGCA |
| | | ACAATTAATAGACTGGATGGAGGCGGATAAAGTTGCAGGACCACTTCTG |
| | | CGCTCGGCCCTTCCGGCTGGCTGGTTTATTGCTGATAAATCTGGAGCCGG |
| | | TGAGCGTGGGTCTCGCGGTATCATTGCAGCACTGGGGCCAGATGGTAAGC |
| | | CCTCCCGTATCGTAGTTATCTACACGACGGGGAGTCAGGCAACTATGGAT |
| | | GAACGAAATAGACAGATCGCTGAGATAGGTGCCTCACTGATTAAGCATT |
| | | GGTAACTGTCAGACCAAGTTTACTCATATATACTTTAGATTGATTTAAAA |
| | | CTTCATTTTTAATTTAAAAGGATCTAGGTGAAGATCCTTTTTGATAATCTC |
| | | ATGACCAAAATCCCTTAACGTGAGTTTTCGTTCCACTGAGCGTCAGACCC |
| | | CGTAGAAAAGATCAAAGGATCTTCTTGAGATCCTTTTTTTCTGCGCGTAAT |
| | | CTGCTGCTTGCAAACAAAAAAACCACCGCTACCAGCGGTGGTTTGTTTGC |
| | | CGGATCAAGAGCTACCAACTCTTTTTCCGAAGGTAACTGGCTTCAGCAGA |
| | | GCGCAGATACCAAATACTGTCCTTCTAGTGTAGCCGTAGTTAGGCCACCA |
| | | CTTCAAGAACTCTGTAGCACCGCCTACATACCTCGCTCTGCTAATCCTGTT |
| | | ACCAGTGGCTGCTGCCAGTGGCGATAAGTCGTGTCTTACCGGGTTGGACT |
| | | CAAGACGATAGTTACCGGATAAGGCGCAGCGGTCGGGCTGAACGGGGGG |
| | | TTCGTGCACACAGCCCAGCTTGGAGCGAACGACCTACACCGAACTGAGAT |
| | | ACCTACAGCGTGAGCTATGAGAAAGCGCCACGCTTCCCGAAGGGAGAAA |
| | | GGCGGACAGGTATCCGGTAAGCGGCAGGGTCGGAACAGGAGAGCGCACG |
| | | AGGGAGCTTCCAGGGGGAAACGCCTGGTATCTTTATAGTCCTGTCGGGTT |
| | | TCGCCACCTCTGACTTGAGCGTCGATTTTTGTGATGCTCGTCAGGGGGGC |
| | | GGAGCCTATGGAAAAACGCCAGCAACGCGGCCTTTTTACGGTTCCTGGCC |
| | | TTTTGCTGGCCTTTTGCTCACATGTTCTTTCCTGCGTTATCCCCTGATTCTG |
| | | TGGATAACCGTATTACCGCCTTTGAGTGAGCTGATACCGCTCGCCGCAGC |
| | | CGAACGACCGAGCGCAGCGAGTCAGTGAGCGAGGAAGCGGAAGAGCGC |
| | | CCAATACGCAAACCGCCTCTCCCCGCGCGTTGGCCGATTCATTAATGCAG |
| | | CTGGCACGACAGGTTTCCCGACTGGAAAGCGGGCAGTGAGCGCAACGCA |
| | | ATTAATGTGAGTTAGCTCACTCATTAGGCACCCCAGGCTTTACACTTTATG |
| | | CTTCCGGCTCGTATGTTGTGTGGAATTGTGAGCGGATAACAATTTCACAC |
| | | AGGAAACAGCTATGACCATGATTACGCCAAGCGCGCAATTAACCCTCACT |
| | | AAAGGGAACAAAAGCTGGAGCTGCA |
| 68 | Codon- | ATGCCTAATCCTCGGCCTGGAAAGCCTAGCGCTCCTTCTCTTGCTCTGGGA |
| | optimized | CCTTCTCCTGGCGCCTCTCCATCTTGGAGAGCCGCTCCTAAAGCCAGCGA |
| | human FOXP3 | TCTGCTGGGAGCTAGAGGACCTGGCGGCACATTTCAGGGCAGAGATCTTA |
| | cDNA, Without | GAGGCGGAGCCCACGCTAGCTCCTCCAGCCTTAATCCTATGCCTCCTAGC |
| | stop codon | CAGCTCCAGCTGCCTACACTGCCTCTGGTTATGGTGGCTCCTAGCGGAGC |
| | | TAGACTGGGCCCTCTGCCTCATCTGCAAGCTCTGCTGCAGGACAGACCCC |
| | | ACTTCATGCACCAGCTGAGCACCGTGGATGCCCACGCAAGAACACCTGTG |
| | | CTGCAGGTTCACCCTCTGGAATCCCCAGCCATGATCAGCCTGACACCTCC |
| | | AACAACAGCCACCGGCGTGTTCAGCCTGAAAGCCAGACCTGGACTGCCTC |
| | | CTGGCATCAATGTGGCCAGCCTGGAATGGGTGTCCAGAGAACCTGCTCTG |
| | | CTGTGCACATTCCCCAATCCAAGCGCTCCCAGAAAGGACAGCACACTGTC |
| | | TGCCGTGCCTCAGAGCAGCTATCCCCTGCTTGCTAACGGCGTGTGCAAGT |
| | | GGCCTGGATGCGAGAAGGTGTTCGAGGAACCCGAGGACTTCCTGAAGCA |
| | | CTGCCAGGCCGATCATCTGCTGGACGAGAAAGGCAGAGCCCAGTGTCTG |
| | | CTCCAGCGCGAGATGGTGCAGTCTCTGGAACAGCAGCTGGTCCTGGAAA |
| | | AAGAAAAGCTGAGCGCCATGCAGGCCCACCTGGCCGGAAAAATGGCCCT |
| | | GACAAAGGCCAGCAGCGTGGCCTCTTCTGATAAGGGCAGCTGCTGCATTG |
| | | TGGCCGCTGGATCTCAGGGACCTGTGGTTCCTGCTTGGAGCGGACCTAGA |
| | | GAGGCCCCTGATTCTCTGTTTGCCGTGCGGAGACACCTGTGGGGCTCTCA |
| | | CGGCAACTCTACTTTCCCCGAGTTCCTGCACAACATGGACTACTTCAAGTT |
| | | CCACAACATGCGGCCTCCATTCACCTACGCCACACTGATCAGATGGGCCA |
| | | TTCTGGAAGCCCCTGAGAAGCAGAGAACCCTGAACGAGATCTACCACTG |
| | | GTTTACCCGGATGTTCGCCTTCTTCCGGAATCACCCTGCCACCTGGAAGA |
| | | ACGCCATCCGGCACAATCTGAGCCTGCACAAGTGCTTCGTGCGCGTGGAA |
| | | TCTGAGAAAGGCGCCGTGTGGACAGTGGACGAGCTGGAATTCAGAAAGA |
| | | AGAGAAGCCAGCGGCCTAGCCGGTGCAGCAATCCTACACCTGGACCT |
| 69 | Codon- | ATGCCTAATCCTCGGCCTGGAAAGCCTAGCGCTCCTTCTCTTGCTCTGGGA |
| | optimized | CCTTCTCCTGGCGCCTCTCCATCTTGGAGAGCCGCTCCTAAAGCCAGCGA |
| | human FOXP3 | TCTGCTGGGAGCTAGAGGACCTGGCGGCACATTTCAGGGCAGAGATCTTA |
| | cDNA, With | GAGGCGGAGCCCACGCTAGCTCCTCCAGCCTTAATCCTATGCCTCCTAGC |
| | stop codon | CAGCTCCAGCTGCCTACACTGCCTCTGGTTATGGTGGCTCCTAGCGGAGC |
| | | TAGACTGGGCCCTCTGCCTCATCTGCAAGCTCTGCTGCAGGACAGACCCC |
| | | ACTTCATGCACCAGCTGAGCACCGTGGATGCCCACGCAAGAACACCTGTG |
| | | CTGCAGGTTCACCCTCTGGAATCCCCAGCCATGATCAGCCTGACACCTCC |
| | | AACAACAGCCACCGGCGTGTTCAGCCTGAAAGCCAGACCTGGACTGCCTC |
| | | CTGGCATCAATGTGGCCAGCCTGGAATGGGTGTCCAGAGAACCTGCTCTG |
| | | CTGTGCACATTCCCCAATCCAAGCGCTCCCAGAAAGGACAGCACACTGTC |
| | | TGCCGTGCCTCAGAGCAGCTATCCCCTGCTTGCTAACGGCGTGTGCAAGT |
| | | GGCCTGGATGCGAGAAGGTGTTCGAGGAACCCGAGGACTTCCTGAAGCA |
| | | CTGCCAGGCCGATCATCTGCTGGACGAGAAAGGCAGAGCCCAGTGTCTG |
| | | CTCCAGCGCGAGATGGTGCAGTCTCTGGAACAGCAGCTGGTCCTGGAAA |
| | | AAGAAAAGCTGAGCGCCATGCAGGCCCACCTGGCCGGAAAAATGGCCCT |
| | | GACAAAGGCCAGCAGCGTGGCCTCTTCTGATAAGGGCAGCTGCTGCATTG |
| | | TGGCCGCTGGATCTCAGGGACCTGTGGTTCCTGCTTGGAGCGGACCTAGA |
| | | GAGGCCCCTGATTCTCTGTTTGCCGTGCGGAGACACCTGTGGGGCTCTCA |
| | | CGGCAACTCTACTTTCCCCGAGTTCCTGCACAACATGGACTACTTCAAGTT |
| | | CCACAACATGCGGCCTCCATTCACCTACGCCACACTGATCAGATGGGCCA |
| | | TTCTGGAAGCCCCTGAGAAGCAGAGAACCCTGAACGAGATCTACCACTG |
| | | GTTTACCCGGATGTTCGCCTTCTTCCGGAATCACCCTGCCACCTGGAAGA |
| | | ACGCCATCCGGCACAATCTGAGCCTGCACAAGTGCTTCGTGCGCGTGGAA |
| | | TCTGAGAAAGGCGCCGTGTGGACAGTGGACGAGCTGGAATTCAGAAAGA |
| | | AGAGAAGCCAGCGGCCTAGCCGGTGCAGCAATCCTACACCTGGACCTTG |
| | | A |
| 70 | naked FRB | MEMWHEGLEEASRLYFGERNVKGMFEVLEPLHAMMERGPQTLKETSFNQA |
| | domain | YGRDLMEAQEWCRKYMKSGNVKDLTQAWDLYYHVFRRISK |
| 71 | mutant naked | MEMWHEGLEEASRLYFGERNVKGMFEVLEPLHAMMERGPQTLKETSFNQA |
| | FRB domain | YGRDLMEAQEWCRKYMKSGNVKDLLQAWDLYYHVFRRISK |
| 72 | MND- | GAACAGAGAAACAGGAGAATATGGGCCAAACAGGATATCTGTGGTAAGC |
| | FOXP3cDNA- | AGTTCCTGCCCCGGCTCAGGGCCAAGAACAGTTGGAACAGCAGAATATG |
| | μDISC-SV40 | GGCCAAACAGGATATCTGTGGTAAGCAGTTCCTGCCCCGGCTCAGGGCCA |
| | polyA | AGAACAGATGGTCCCCAGATGCGGTCCCGCCCTCAGCAGTTTCTAGAGAA |
| | | CCATCAGATGTTTCCAGGGTGCCCCAAGGACCTGAAATGACCCTGTGCCT |
| | | TATTTGAACTAACCAATCAGTTCGCTTCTCGCTTCTGTTCGCGCGCTTCTG |
| | | CTCCCCGAGCTCTATATAAGCAGAGCTCGTTTAGTGAACCGTCAGATCGC |
| | | CTGGAGACGCCATCCACGCTGTTTTGACTTCCATAGAAGGATCTCGAGGC |
| | | CACCATGCCTAATCCTCGGCCTGGAAAGCCTAGCGCTCCTTCTCTTGCTCT |
| | | GGGACCTTCTCCTGGCGCCTCTCCATCTTGGAGAGCCGCTCCTAAAGCCA |
| | | GCGATCTGCTGGGAGCTAGAGGACCTGGCGGCACATTTCAGGGCAGAGA |
| | | TCTTAGAGGCGGAGCCCACGCTAGCTCCTCCAGCCTTAATCCTATGCCTC |
| | | CTAGCCAGCTCCAGCTGCCTACACTGCCTCTGGTTATGGTGGCTCCTAGC |
| | | GGAGCTAGACTGGGCCCTCTGCCTCATCTGCAAGCTCTGCTGCAGGACAG |
| | | ACCCCACTTCATGCACCAGCTGAGCACCGTGGATGCCCACGCAAGAACAC |
| | | CTGTGCTGCAGGTTCACCCTCTGGAATCCCCAGCCATGATCAGCCTGACA |
| | | CCTCCAACAACAGCCACCGGCGTGTTCAGCCTGAAAGCCAGACCTGGACT |
| | | GCCTCCTGGCATCAATGTGGCCAGCCTGGAATGGGTGTCCAGAGAACCTG |
| | | CTCTGCTGTGCACATTCCCCAATCCAAGCGCTCCCAGAAAGGACAGCACA |
| | | CTGTCTGCCGTGCCTCAGAGCAGCTATCCCCTGCTTGCTAACGGCGTGTG |
| | | CAAGTGGCCTGGATGCGAGAAGGTGTTCGAGGAACCCGAGGACTTCCTG |
| | | AAGCACTGCCAGGCCGATCATCTGCTGGACGAGAAAGGCAGAGCCCAGT |
| | | GTCTGCTCCAGCGCGAGATGGTGCAGTCTCTGGAACAGCAGCTGGTCCTG |
| | | GAAAAAGAAAAGCTGAGCGCCATGCAGGCCCACCTGGCCGGAAAAATGG |
| | | CCCTGACAAAGGCCAGCAGCGTGGCCTCTTCTGATAAGGGCAGCTGCTGC |
| | | ATTGTGGCCGCTGGATCTCAGGGACCTGTGGTTCCTGCTTGGAGCGGACC |
| | | TAGAGAGGCCCCTGATTCTCTGTTTGCCGTGCGGAGACACCTGTGGGGCT |
| | | CTCACGGCAACTCTACTTTCCCCGAGTTCCTGCACAACATGGACTACTTCA |
| | | AGTTCCACAACATGCGGCCTCCATTCACCTACGCCACACTGATCAGATGG |
| | | GCCATTCTGGAAGCCCCTGAGAAGCAGAGAACCCTGAACGAGATCTACC |
| | | ACTGGTTTACCCGGATGTTCGCCTTCTTCCGGAATCACCCTGCCACCTGGA |
| | | AGAACGCCATCCGGCACAATCTGAGCCTGCACAAGTGCTTCGTGCGCGTG |
| | | GAATCTGAGAAAGGCGCCGTGTGGACAGTGGACGAGCTGGAATTCAGAA |
| | | AGAAGAGAAGCCAGCGGCCTAGCCGGTGCAGCAATCCTACACCTGGACC |
| | | TGGAAGCGGAGCGACTAACTTCAGCCTGCTTAAGCAGGCCGGAGATGTG |
| | | GAGGAAAACCCTGGACCGATGCCTCTGGGCCTGCTGTGGCTGGGCCTGGC |
| | | CCTGCTGGGCGCCCTGCACGCCCAGGCCGGCGTGCAGGTGGAGACAATCT |
| | | CCCCAGGCGACGGACGCACATTCCCTAAGCGGGGCCAGACCTGCGTGGT |
| | | GCACTATACAGGCATGCTGGAGGATGGCAAGAAGTTTGACAGCTCCCGG |
| | | GATAGAAACAAGCCATTCAAGTTTATGCTGGGCAAGCAGGAAGTGATCA |
| | | GAGGCTGGGAGGAGGGCGTGGCCCAGATGTCTGTGGGCCAGAGGGCCAA |
| | | GCTGACCATCAGCCCAGACTACGCCTATGGAGCAACAGGCCACCCAGGA |
| | | ATCATCCCACCTCACGCCACCCTGGTGTTCGATGTGGAGCTGCTGAAGCT |
| | | GGGCGAGGGAGGGTCACCTGGATCCAACACATCAAAAGAGAACCCCTTT |
| | | CTGTTCGCATTGGAGGCCGTAGTCATATCTGTTGGATCCATGGGACTTATT |
| | | ATCTCCCTGTTGTGTGTGTACTTCTGGCTGGAACGGACTATGCCCAGGATC |
| | | CCCACGCTCAAGAATCTGGAAGATCTCGTCACAGAATACCATGGTAATTT |
| | | CAGCGCCTGGAGCGGAGTCTCTAAGGGTCTGGCCGAATCCCTCCAACCCG |
| | | ATTATTCTGAACGGTTGTGCCTCGTATCCGAAATACCACCAAAAGGCGGG |
| | | GCTCTGGGTGAGGGCCCAGGGGCGAGTCCGTGCAATCAACACAGCCCGT |
| | | ATTGGGCCCCTCCTTGTTATACGTTGAAGCCCGAAACTGGAAGCGGAGCT |
| | | ACTAACTTCAGCCTGCTGAAGCAGGCTGGAGACGTGGAGGAGAACCCTG |
| | | GACCTATGGCACTGCCCGTGACCGCCCTGCTGCTGCCTCTGGCCCTGCTG |
| | | CTGCACGCAGCCCGGCCTATCCTGTGGCACGAGATGTGGCACGAGGGCCT |
| | | GGAGGAGGCCAGCAGGCTGTATTTTGGCGAGCGCAACGTGAAGGGCATG |
| | | TTCGAGGTGCTGGAGCCTCTGCACGCCATGATGGAGAGAGGCCCACAGA |
| | | CCCTGAAGGAGACATCCTTTAACCAGGCCTATGGACGGGACCTGATGGA |
| | | GGCACAGGAGTGGTGCAGAAAGTACATGAAGTCTGGCAATGTGAAGGAC |
| | | CTGCTGCAGGCCTGGGATCTGTACTATCACGTGTTTCGGAGAATCTCCAA |
| | | GCCAGCAGCTCTCGGCAAAGACACGATTCCGTGGCTTGGGCATCTGCTCG |
| | | TTGGGCTGAGCGGTGCGTTTGGTTTCATCATCTTGGTCTATCTCTTGATCA |
| | | ATTGCAGAAATACAGGCCCTTGGCTGAAAAAAGTGCTCAAGTGTAATACC |
| | | CCCGACCCAAGCAAGTTCTTCTCCCAGCTTTCTTCAGAGCATGGAGGCGA |
| | | TGTGCAGAAATGGCTCTCTTCACCTTTTCCCTCCTCAAGCTTCTCCCCGGG |
| | | AGGGCTGGCGCCCGAGATTTCACCTCTTGAGGTACTTGAACGAGACAAGG |
| | | TTACCCAACTTCTCCTTCAACAGGATAAGGTACCCGAACCTGCGAGCCTT |
| | | AGCTTGAATACAGACGCTTATCTCTCACTGCAGGAACTGCAAGGATCTGG |
| | | TGCTACTAATTTTTCTCTTTTGAAGCAAGCTGGAGATGTTGAAGAGAACC |
| | | CCGGTCCGGAGATGTGGCATGAGGGTCTGGAAGAAGCGTCTCGACTGTA |
| | | CTTTGGTGAGCGCAATGTGAAGGGCATGTTTGAAGTCCTCGAACCCCTTC |
| | | ATGCCATGATGGAACGCGGACCCCAGACCTTGAAGGAGACAAGTTTTAA |
| | | CCAAGCTTACGGAAGAGACCTGATGGAAGCCCAGGAATGGTGCAGGAAA |
| | | TACATGAAAAGCGGGAATGTGAAGGACTTGCTCCAAGCGTGGGACCTGT |
| | | ACTATCATGTCTTTAGGCGCATTAGTAAGTGAGTCGACTGCTTTATTTGTG |
| | | AAATTTGTGATGCTATTGCTTTATTTGTAACCATTATAAGCTGCAATAAAC |
| | | AAGTTAACAACAACAATTGCATTCATTTTATGTTTCAGGTTCAGGGGGAG |
| | | ATGTGGGAGGTTTTTTAAAGC |
| 73 | FOXP3cDNA- | MPNPRPGKPSAPSLALGPSPGASPSWRAAPKASDLLGARGPGGTFQGRDLRG |
| | μDISC amino | GAHASSSSLNPMPPSQLQLPTLPLVMVAPSGARLGPLPHLQALLQDRPHFMH |
| | acid sequence | QLSTVDAHARTPVLQVHPLESPAMISLTPPTTATGVFSLKARPGLPPGINVAS |
| | | LEWVSREPALLCTFPNPSAPRKDSTLSAVPQSSYPLLANGVCKWPGCEKVFE |
| | | EPEDFLKHCQADHLLDEKGRAQCLLQREMVQSLEQQLVLEKEKLSAMQAH |
| | | LAGKMALTKASSVASSDKGSCCIVAAGSQGPVVPAWSGPREAPDSLFAVRR |
| | | HLWGSHGNSTFPEFLHNMDYFKFHNMRPPFTYATLIRWAILEAPEKQRTLNE |
| | | IYHWFTRMFAFFRNHPATWKNAIRHNLSLHKCFVRVESEKGAVWTVDELEF |
| | | RKKRSQRPSRCSNPTPGPGSGATNFSLLKQAGDVEENPGPMPLGLLWLGLAL |
| | | LGALHAQAGVQVETISPGDGRTFPKRGQTCVVHYTGMLEDGKKFDSSRDRN |
| | | KPFKFMLGKQEVIRGWEEGVAQMSVGQRAKLTISPDYAYGATGHPGIIPPHA |
| | | TLVFDVELLKLGEGGSPGSNTSKENPFLFALEAVVISVGSMGLIISLLCVYFW |
| | | LERTMPRIPTLKNLEDLVTEYHGNFSAWSGVSKGLAESLQPDYSERLCLVSEI |
| | | PPKGGALGEGPGASPCNQHSPYWAPPCYTLKPETGSGATNFSLLKQAGDVEE |
| | | NPGPMALPVTALLLPLALLLHAARPILWHEMWHEGLEEASRLYFGERNVKG |
| | | MFEVLEPLHAMMERGPQTLKETSFNQAYGRDLMEAQEWCRKYMKSGNVK |
| | | DLLQAWDLYYHVFRRISKPAALGKDTIPWLGHLLVGLSGAFGFIILVYLLINC |
| | | RNTGPWLKKVLKCNTPDPSKFFSQLSSEHGGDVQKWLSSPFPSSSFSPGGLAP |
| | | EISPLEVLERDKVTQLLLQQDKVPEPASLSLNTDAYLSLQELQGSGATNFSLL |
| | | KQAGDVEENPGPEMWHEGLEEASRLYFGERNVKGMFEVLEPLHAMMERGP |
| | | QTLKETSFNQAYGRDLMEAQEWCRKYMKSGNVKDLLQAWDLYYHVFRRIS |
| | | K* |
| 74 | FOXP3cDNA- | MPNPRPGKPSAPSLALGPSPGASPSWRAAPKASDLLGARGPGGTFQGRDLRG |
| | LNGFRe-μDISC | GAHASSSSLNPMPPSQLQLPTLPLVMVAPSGARLGPLPHLQALLQDRPHFMH |
| | amino acid | QLSTVDAHARTPVLQVHPLESPAMISLTPPTTATGVFSLKARPGLPPGINVAS |
| | sequence | LEWVSREPALLCTFPNPSAPRKDSTLSAVPQSSYPLLANGVCKWPGCEKVFE |
| | | EPEDFLKHCQADHLLDEKGRAQCLLQREMVQSLEQQLVLEKEKLSAMQAH |
| | | LAGKMALTKASSVASSDKGSCCIVAAGSQGPVVPAWSGPREAPDSLFAVRR |
| | | HLWGSHGNSTFPEFLHNMDYFKFHNMRPPFTYATLIRWAILEAPEKQRTLNE |
| | | IYHWFTRMFAFFRNHPATWKNAIRHNLSLHKCFVRVESEKGAVWTVDELEF |
| | | RKKRSQRPSRCSNPTPGPGSGATNFSLLKQAGDVEENPGPMPLGLLWLGLAL |
| | | LGALHAQAMGAGATGRAMDGPRLLLLLLLGVSLGGAKEACPTGLYTHSGE |
| | | CCKACNLGEGVAQPCGANQTVCEPCLDSVTFSDVVSATEPCKPCTECVGLQS |
| | | MSAPCVEADDAVCRCAYGYYQDETTGRCEACRVCEAGSGLVFSCQDKQNT |
| | | VCEECPDGTYSDEANHVDPCLPCTVCEDTERQLRECTRWADAECEEIPGRWI |
| | | TRSTPPEGSDSTAPSTQEPEAPPEQDLIASTVAGVVTTVMGSSQPVVTRGTTD |
| | | NLIPVYCSILAAVVVGLVAYIAFKRGVQVETISPGDGRTFPKRGQTCVVHYT |
| | | GMLEDGKKFDSSRDRNKPFKFMLGKQEVIRGWEEGVAQMSVGQRAKLTISP |
| | | DYAYGATGHPGIIPPHATLVFDVELLKLGEGGSPGSNTSKENPFLFALEAVVI |
| | | SVGSMGLIISLLCVYFWLERTMPRIPTLKNLEDLVTEYHGNFSAWSGVSKGL |
| | | AESLQPDYSERLCLVSEIPPKGGALGEGPGASPCNQHSPYWAPPCYTLKPETG |
| | | SGATNFSLLKQAGDVEENPGPMALPVTALLLPLALLLHAARPILWHEMWHE |
| | | GLEEASRLYFGERNVKGMFEVLEPLHAMMERGPQTLKETSFNQAYGRDLME |
| | | AQEWCRKYMKSGNVKDLLQAWDLYYHVFRRISKPAALGKDTIPWLGHLLV |
| | | GLSGAFGFIILVYLLINCRNTGPWLKKVLKCNTPDPSKFFSQLSSEHGGDVQK |
| | | WLSSPFPSSSFSPGGLAPEISPLEVLERDKVTQLLLQQDKVPEPASLSLNTDAY |
| | | LSLQELQGSGATNFSLLKQAGDVEENPGPEMWHEGLEEASRLYFGERNVKG |
| | | MFEVLEPLHAMMERGPQTLKETSFNQAYGRDLMEAQEWCRKYMKSGNVK |
| | | DLLQAWDLYYHVFRRISK |
| 75 | μDISC- | MPLGLLWLGLALLGALHAQAGVQVETISPGDGRTFPKRGQTCVVHYTGMLE |
| | FOXP3cDNA | DGKKFDSSRDRNKPFKFMLGKQEVIRGWEEGVAQMSVGQRAKLTISPDYAY |
| | amino acid | GATGHPGIIPPHATLVFDVELLKLGEGGSPGSNTSKENPFLFALEAVVISVGS |
| | sequence | MGLIISLLCVYFWLERTMPRIPTLKNLEDLVTEYHGNFSAWSGVSKGLAESL |
| | | QPDYSERLCLVSEIPPKGGALGEGPGASPCNQHSPYWAPPCYTLKPETGSGAT |
| | | NFSLLKQAGDVEENPGPMALPVTALLLPLALLLHAARPILWHEMWHEGLEE |
| | | ASRLYFGERNVKGMFEVLEPLHAMMERGPQTLKETSFNQAYGRDLMEAQE |
| | | WCRKYMKSGNVKDLLQAWDLYYHVFRRISKPAALGKDTIPWLGHLLVGLS |
| | | GAFGFIILVYLLINCRNTGPWLKKVLKCNTPDPSKFFSQLSSEHGGDVQKWLS |
| | | SPFPSSSFSPGGLAPEISPLEVLERDKVTQLLLQQDKVPEPASLSLNTDAYLSL |
| | | QELQGSGATNFSLLKQAGDVEENPGPEMWHEGLEEASRLYFGERNVKGMFE |
| | | VLEPLHAMMERGPQTLKETSFNQAYGRDLMEAQEWCRKYMKSGNVKDLL |
| | | QAWDLYYHVFRRISKGSGATNFSLLKQAGDVEENPGPMPNPRPGKPSAPSLA |
| | | LGPSPGASPSWRAAPKASDLLGARGPGGTFQGRDLRGGAHASSSSLNPMPPS |
| | | QLQLPTLPLVMVAPSGARLGPLPHLQALLQDRPHFMHQLSTVDAHARTPVL |
| | | QVHPLESPAMISLTPPTTATGVFSLKARPGLPPGINVASLEWVSREPALLCTFP |
| | | NPSAPRKDSTLSAVPQSSYPLLANGVCKWPGCEKVFEEPEDFLKHCQADHLL |
| | | DEKGRAQCLLQREMVQSLEQQLVLEKEKLSAMQAHLAGKMALTKASSVAS |
| | | SDKGSCCIVAAGSQGPVVPAWSGPREAPDSLFAVRRHLWGSHGNSTFPEFLH |
| | | NMDYFKFHNMRPPFTYATLIRWAILEAPEKQRTLNEIYHWFTRMFAFFRNHP |
| | | ATWKNAIRHNLSLHKCFVRVESEKGAVWTVDELEFRKKRSQRPSRCSNPTP |
| | | GP |
| 76 | LNGFRe-μDISC | MPLGLLWLGLALLGALHAQAMGAGATGRAMDGPRLLLLLLLGVSLGGAKE |
| | -FOXP3cDNA | ACPTGLYTHSGECCKACNLGEGVAQPCGANQTVCEPCLDSVTFSDVVSATEP |
| | amino acid | CKPCTECVGLQSMSAPCVEADDAVCRCAYGYYQDETTGRCEACRVCEAGS |
| | sequence | GLVFSCQDKQNTVCEECPDGTYSDEANHVDPCLPCTVCEDTERQLRECTRW |
| | | ADAECEEIPGRWITRSTPPEGSDSTAPSTQEPEAPPEQDLIASTVAGVVTTVM |
| | | GSSQPVVTRGTTDNLIPVYCSILAAVVVGLVAYIAFKRGVQVETISPGDGRTF |
| | | PKRGQTCVVHYTGMLEDGKKFDSSRDRNKPFKFMLGKQEVIRGWEEGVAQ |
| | | MSVGQRAKLTISPDYAYGATGHPGIIPPHATLVFDVELLKLGEGGSPGSNTSK |
| | | ENPFLFALEAVVISVGSMGLIISLLCVYFWLERTMPRIPTLKNLEDLVTEYHG |
| | | NFSAWSGVSKGLAESLQPDYSERLCLVSEIPPKGGALGEGPGASPCNQHSPY |
| | | WAPPCYTLKPETGSGATNFSLLKQAGDVEENPGPMALPVTALLLPLALLLHA |
| | | ARPILWHEMWHEGLEEASRLYFGERNVKGMFEVLEPLHAMMERGPQTLKE |
| | | TSFNQAYGRDLMEAQEWCRKYMKSGNVKDLLQAWDLYYHVFRRISKPAAL |
| | | GKDTIPWLGHLLVGLSGAFGFIILVYLLINCRNTGPWLKKVLKCNTPDPSKFF |
| | | SQLSSEHGGDVQKWLSSPFPSSSFSPGGLAPEISPLEVLERDKVTQLLLQQDK |
| | | VPEPASLSLNTDAYLSLQELQGSGATNFSLLKQAGDVEENPGPEMWHEGLEE |
| | | ASRLYFGERNVKGMFEVLEPLHAMMERGPQTLKETSFNQAYGRDLMEAQE |
| | | WCRKYMKSGNVKDLLQAWDLYYHVFRRISKGSGATNFSLLKQAGDVEENP |
| | | GPMPNPRPGKPSAPSLALGPSPGASPSWRAAPKASDLLGARGPGGTFQGRDL |
| | | RGGAHASSSSLNPMPPSQLQLPTLPLVMVAPSGARLGPLPHLQALLQDRPHF |
| | | MHQLSTVDAHARTPVLQVHPLESPAMISLTPPTTATGVFSLKARPGLPPGINV |
| | | ASLEWVSREPALLCTFPNPSAPRKDSTLSAVPQSSYPLLANGVCKWPGCEKV |
| | | FEEPEDFLKHCQADHLLDEKGRAQCLLQREMVQSLEQQLVLEKEKLSAMQA |
| | | HLAGKMALTKASSVASSDKGSCCIVAAGSQGPVVPAWSGPREAPDSLFAVR |
| | | RHLWGSHGNSTFPEFLHNMDYFKFHNMRPPFTYATLIRWAILEAPEKQRTLN |
| | | EIYHWFTRMFAFFRNHPATWKNAIRHNLSLHKCFVRVESEKGAVWTVDELE |
| | | FRKKRSQRPSRCSNPTPGP* |
| 77 | DISC amino | MPLGLLWLGLALLGALHAQAGVQVETISPGDGRTFPKRGQTCVVHYTGMLE |
| | acid sequence | DGKKFDSSRDRNKPFKFMLGKQEVIRGWEEGVAQMSVGQRAKLTISPDYAY |
| | | GATGHPGIIPPHATLVFDVELLKLGEGGSPGSNTSKENPFLFALEAVVISVGS |
| | | MGLIISLLCVYFWLERTMPRIPTLKNLEDLVTEYHGNFSAWSGVSKGLAESL |
| | | QPDYSERLCLVSEIPPKGGALGEGPGASPCNQHSPYWAPPCYTLKPETGSGAT |
| | | NFSLLKQAGDVEENPGPMALPVTALLLPLALLLHAARPILWHEMWHEGLEE |
| | | ASRLYFGERNVKGMFEVLEPLHAMMERGPQTLKETSFNQAYGRDLMEAQE |
| | | WCRKYMKSGNVKDLLQAWDLYYHVFRRISKPAALGKDTIPWLGHLLVGLS |
| | | GAFGFIILVYLLINCRNTGPWLKKVLKCNTPDPSKFFSQLSSEHGGDVQKWLS |
| | | SPFPSSSFSPGGLAPEISPLEVLERDKVTQLLLQQDKVPEPASLSSNHSLTSCFT |
| | | NQGYFFFHLPDALEIEACQVYFTYDPYSEEDPDEGVAGAPTGSSPQPLQPLSG |
| | | EDDAYCTFPSRDDLLLFSPSLLGGPSPPSTAPGGSGAGEERMPPSLQERVPRD |
| | | WDPQPLGPPTPGVPDLVDFQPPPELVLREAGEEVPDAGPREGVSFPWSRPPG |
| | | QGEFRALNARLPLNTDAYLSLQELQGQDPTHLVGSGATNFSLLKQAGDVEE |
| | | NPGPEMWHEGLEEASRLYFGERNVKGMFEVLEPLHAMMERGPQTLKETSFN |
| | | QAYGRDLMEAQEWCRKYMKSGNVKDLLQAWDLYYHVFRRISK |
| 78 | μDISC amino | MPLGLLWLGLALLGALHAQAGVQVETISPGDGRTFPKRGQTCVVHYTGMLE |
| | acid sequence | DGKKFDSSRDRNKPFKFMLGKQEVIRGWEEGVAQMSVGQRAKLTISPDYAY |
| | | GATGHPGIIPPHATLVFDVELLKLGEGGSPGSNTSKENPFLFALEAVVISVGS |
| | | MGLIISLLCVYFWLERTMPRIPTLKNLEDLVTEYHGNFSAWSGVSKGLAESL |
| | | QPDYSERLCLVSEIPPKGGALGEGPGASPCNQHSPYWAPPCYTLKPETGSGAT |
| | | NFSLLKQAGDVEENPGPMALPVTALLLPLALLLHAARPILWHEMWHEGLEE |
| | | ASRLYFGERNVKGMFEVLEPLHAMMERGPQTLKETSFNQAYGRDLMEAQE |
| | | WCRKYMKSGNVKDLLQAWDLYYHVFRRISKPAALGKDTIPWLGHLLVGLS |
| | | GAFGFIILVYLLINCRNTGPWLKKVLKCNTPDPSKFFSQLSSEHGGDVQKWLS |
| | | SPFPSSSFSPGGLAPEISPLEVLERDKVTQLLLQQDKVPEPASLSLNTDAYLSL |
| | | QELQGSGATNFSLLKQAGDVEENPGPEMWHEGLEEASRLYFGERNVKGMFE |
| | | VLEPLHAMMERGPQTLKETSFNQAYGRDLMEAQEWCRKYMKSGNVKDLL |
| | | QAWDLYYHVFRRISK |
| 79 | CISCβ-DN | MALPVTALLLPLALLLHAARPILWHEMWHEGLEEASRLYFGERNVKGMFEV |
| | amino acid | LEPLHAMMERGPQTLKETSFNQAYGRDLMEAQEWCRKYMKSGNVKDLLQ |
| | sequence | AWDLYYHVFRRISKPAALGKDTIPWLGHLLVGLSGAFGFIILVYLLINCRNTG |
| | | PWLKKVLKCNTPDPSKFFSQLSSEHGGDVQKWLSSPFPSSSFSPGGLAPEISPL |
| | | EVLERDKVTQLLLQQDKVPEPASLSSNHSLTSCFTNQGYFFFHLPDALEIEAC |
| | | QVYFTYDPYSEEDPDEGVAGAPTGSSPQPLQPLSGEDDAYCTFPSRDDLLLFS |
| | | PSLLGGPSPPSTAPGGSGAGEERMPPSLQERVPRDWDPQPLGPPTPGVPDLVD |
| | | FQPPPELVLREAGEEVPDAGPREGVSFPWSRPPGQGEFRALNARLPLNTDAY |
| | | LSLQELQGQDPTHLVGSGATNFSLLKQAGDVEENPGPEMWHEGLEEASRLY |
| | | FGERNVKGMFEVLEPLHAMMERGPQTLKETSFNQAYGRDLMEAQEWCRKY |
| | | MKSGNVKDLLQAWDLYYHVFRRISK |
| 80 | CISCγ- | MPLGLLWLGLALLGALHAQAGVQVETISPGDGRTFPKRGQTCVVHYTGMLE |
| | FOXP3cDNA- | DGKKFDSSRDRNKPFKFMLGKQEVIRGWEEGVAQMSVGQRAKLTISPDYAY |
| | LNGFR amino | GATGHPGIIPPHATLVFDVELLKLGEGGSPGSNTSKENPFLFALEAVVISVGS |
| | acid sequence | MGLIISLLCVYFWLERTMPRIPTLKNLEDLVTEYHGNFSAWSGVSKGLAESL |
| | | QPDYSERLCLVSEIPPKGGALGEGPGASPCNQHSPYWAPPCYTLKPETGSGAT |
| | | NFSLLKQAGDVEENPGPMPNPRPGKPSAPSLALGPSPGASPSWRAAPKASDL |
| | | LGARGPGGTFQGRDLRGGAHASSSSLNPMPPSQLQLPTLPLVMVAPSGARLG |
| | | PLPHLQALLQDRPHFMHQLSTVDAHARTPVLQVHPLESPAMISLTPPTTATG |
| | | VFSLKARPGLPPGINVASLEWVSREPALLCTFPNPSAPRKDSTLSAVPQSSYPL |
| | | LANGVCKWPGCEKVFEEPEDFLKHCQADHLLDEKGRAQCLLQREMVQSLE |
| | | QQLVLEKEKLSAMQAHLAGKMALTKASSVASSDKGSCCIVAAGSQGPVVPA |
| | | WSGPREAPDSLFAVRRHLWGSHGNSTFPEFLHNMDYFKFHNMRPPFTYATLI |
| | | RWAILEAPEKQRTLNEIYHWFTRMFAFFRNHPATWKNAIRHNLSLHKCFVR |
| | | VESEKGAVWTVDELEFRKKRSQRPSRCSNPTPGPGSGATNFSLLKQAGDVEE |
| | | NPGPMGAGATGRAMDGPRLLLLLLLGVSLGGAKEACPTGLYTHSGECCKAC |
| | | NLGEGVAQPCGANQTVCEPCLDSVTFSDVVSATEPCKPCTECVGLQSMSAPC |
| | | VEADDAVCRCAYGYYQDETTGRCEACRVCEAGSGLVFSCQDKQNTVCEEC |
| | | PDGTYSDEANHVDPCLPCTVCEDTERQLRECTRWADAECEDPGRWITRSTP |
| | | PEGSDSTAPSTQEPEAPPEQDLIASTVAGVVTTVMGSSQPVVTRGTTDNLIPV |
| | | YCSILAAVVVGLVAYIAFKR* |
| 81 | CISCγ-LNGFR- | ATGCCTCTGGGCCTGCTGTGGCTGGGCCTGGCCCTGCTGGGCGCCCTGCA |
| | FOXP3cDNA | CGCCCAGGCCGGCGTGCAGGTGGAGACAATCTCCCCAGGCGACGGACGC |
| | | ACATTCCCTAAGCGGGGCCAGACCTGCGTGGTGCACTATACAGGCATGCT |
| | | GGAGGATGGCAAGAAGTTTGACAGCTCCCGGGATAGAAACAAGCCATTC |
| | | AAGTTTATGCTGGGCAAGCAGGAAGTGATCAGAGGCTGGGAGGAGGGCG |
| | | TGGCCCAGATGTCTGTGGGCCAGAGGGCCAAGCTGACCATCAGCCCAGA |
| | | CTACGCCTATGGAGCAACAGGCCACCCAGGAATCATCCCACCTCACGCCA |
| | | CCCTGGTGTTCGATGTGGAGCTGCTGAAGCTGGGCGAGGGAGGGTCACCT |
| | | GGATCCAACACATCAAAAGAGAACCCCTTTCTGTTCGCATTGGAGGCCGT |
| | | AGTCATATCTGTTGGATCCATGGGACTTATTATCTCCCTGTTGTGTGTGTA |
| | | CTTCTGGCTGGAACGGACTATGCCCAGGATCCCCACGCTCAAGAATCTGG |
| | | AAGATCTCGTCACAGAATACCATGGTAATTTCAGCGCCTGGAGCGGAGTC |
| | | TCTAAGGGTCTGGCCGAATCCCTCCAACCCGATTATTCTGAACGGTTGTG |
| | | CCTCGTATCCGAAATACCACCAAAAGGCGGGGCTCTGGGTGAGGGCCCA |
| | | GGGGCGAGTCCGTGCAATCAACACAGCCCGTATTGGGCCCCTCCTTGTTA |
| | | TACGTTGAAGCCCGAAACTGGAAGCGGAGCGACTAACTTCAGCCTGCTTA |
| | | AGCAGGCCGGAGATGTGGAGGAAAACCCTGGACCGATGGGGGCAGGTGC |
| | | CACCGGACGAGCCATGGACGGGCCGCGCCTGCTGCTGTTGCTGCTTCTGG |
| | | GGGTGTCCCTTGGAGGTGCCAAGGAGGCATGCCCCACAGGCCTGTACAC |
| | | ACACAGCGGTGAGTGCTGCAAAGCCTGCAACCTGGGCGAGGGTGTGGCC |
| | | CAGCCTTGTGGAGCCAACCAGACCGTGTGTGAGCCCTGCCTGGACAGCGT |
| | | GACGTTCTCCGACGTGGTGAGCGCGACCGAGCCGTGCAAGCCGTGCACC |
| | | GAGTGCGTGGGGCTCCAGAGCATGTCGGCGCCGTGCGTGGAGGCCGACG |
| | | ACGCCGTGTGCCGCTGCGCCTACGGCTACTACCAGGATGAGACGACTGGG |
| | | CGCTGCGAGGCGTGCCGCGTGTGCGAGGCGGGCTCGGGCCTCGTGTTCTC |
| | | CTGCCAGGACAAGCAGAACACCGTGTGCGAGGAGTGCCCCGACGGCACG |
| | | TATTCCGACGAGGCCAACCACGTGGACCCGTGCCTGCCCTGCACCGTGTG |
| | | CGAGGACACCGAGCGCCAGCTCCGCGAGTGCACACGCTGGGCCGACGCC |
| | | GAGTGCGAGGAGATCCCTGGCCGTTGGATTACACGGTCCACACCCCCAGA |
| | | GGGCTCGGACAGCACAGCCCCCAGCACCCAGGAGCCTGAGGCACCTCCA |
| | | GAACAAGACCTCATAGCCAGCACGGTGGCAGGTGTGGTGACCACAGTGA |
| | | TGGGCAGCTCCCAGCCCGTGGTGACCCGAGGCACCACCGACAACCTCATC |
| | | CCTGTCTATTGCTCCATCCTGGCTGCTGTGGTTGTGGGTCTTGTGGCCTAC |
| | | ATAGCCTTCAAGAGGGGAAGCGGAGCGACTAACTTCAGCCTGCTGAAGC |
| | | AGGCCGGAGATGTGGAGGAAAACCCTGGACCGATGCCTAATCCTCGGCC |
| | | TGGAAAGCCTAGCGCTCCTTCTCTTGCTCTGGGACCTTCTCCTGGCGCCTC |
| | | TCCATCTTGGAGAGCCGCTCCTAAAGCCAGCGATCTGCTGGGAGCTAGAG |
| | | GACCTGGCGGCACATTTCAGGGCAGAGATCTTAGAGGCGGAGCCCACGC |
| | | TAGCTCCTCCAGCCTTAATCCTATGCCTCCTAGCCAGCTCCAGCTGCCTAC |
| | | ACTGCCTCTGGTTATGGTGGCTCCTAGCGGAGCTAGACTGGGCCCTCTGC |
| | | CTCATCTGCAAGCTCTGCTGCAGGACAGACCCCACTTCATGCACCAGCTG |
| | | AGCACCGTGGATGCCCACGCAAGAACACCTGTGCTGCAGGTTCACCCTCT |
| | | GGAATCCCCAGCCATGATCAGCCTGACACCTCCAACAACAGCCACCGGC |
| | | GTGTTCAGCCTGAAAGCCAGACCTGGACTGCCTCCTGGCATCAATGTGGC |
| | | CAGCCTGGAATGGGTGTCCAGAGAACCTGCTCTGCTGTGCACATTCCCCA |
| | | ATCCAAGCGCTCCCAGAAAGGACAGCACACTGTCTGCCGTGCCTCAGAGC |
| | | AGCTATCCCCTGCTTGCTAACGGCGTGTGCAAGTGGCCTGGATGCGAGAA |
| | | GGTGTTCGAGGAACCCGAGGACTTCCTGAAGCACTGCCAGGCCGATCATC |
| | | TGCTGGACGAGAAAGGCAGAGCCCAGTGTCTGCTCCAGCGCGAGATGGT |
| | | GCAGTCTCTGGAACAGCAGCTGGTCCTGGAAAAAGAAAAGCTGAGCGCC |
| | | ATGCAGGCCCACCTGGCCGGAAAAATGGCCCTGACAAAGGCCAGCAGCG |
| | | TGGCCTCTTCTGATAAGGGCAGCTGCTGCATTGTGGCCGCTGGATCTCAG |
| | | GGACCTGTGGTTCCTGCTTGGAGCGGACCTAGAGAGGCCCCTGATTCTCT |
| | | GTTTGCCGTGCGGAGACACCTGTGGGGCTCTCACGGCAACTCTACTTTCC |
| | | CCGAGTTCCTGCACAACATGGACTACTTCAAGTTCCACAACATGCGGCCT |
| | | CCATTCACCTACGCCACACTGATCAGATGGGCCATTCTGGAAGCCCCTGA |
| | | GAAGCAGAGAACCCTGAACGAGATCTACCACTGGTTTACCCGGATGTTCG |
| | | CCTTCTTCCGGAATCACCCTGCCACCTGGAAGAACGCCATCCGGCACAAT |
| | | CTGAGCCTGCACAAGTGCTTCGTGCGCGTGGAATCTGAGAAAGGCGCCGT |
| | | GTGGACAGTGGACGAGCTGGAATTCAGAAAGAAGAGAAGCCAGCGGCCT |
| | | AGCCGGTGCAGCAATCCTACACCTGGACCTTGA |
| 82 | CISCγ: FKBP- | ATGCCTCTGGGCCTGCTGTGGCTGGGCCTGGCCCTGCTGGGCGCCCTGCA |
| | IL2Rγ; | CGCCCAGGCCGGCGTGCAGGTGGAGACAATCTCCCCAGGCGACGGACGC |
| | nucleotide | ACATTCCCTAAGCGGGGCCAGACCTGCGTGGTGCACTATACAGGCATGCT |
| | sequence | GGAGGATGGCAAGAAGTTTGACAGCTCCCGGGATAGAAACAAGCCATTC |
| | | AAGTTTATGCTGGGCAAGCAGGAAGTGATCAGAGGCTGGGAGGAGGGCG |
| | | TGGCCCAGATGTCTGTGGGCCAGAGGGCCAAGCTGACCATCAGCCCAGA |
| | | CTACGCCTATGGAGCAACAGGCCACCCAGGAATCATCCCACCTCACGCCA |
| | | CCCTGGTGTTCGATGTGGAGCTGCTGAAGCTGGGCGAGGGAGGGTCACCT |
| | | GGATCCAACACATCAAAAGAGAACCCCTTTCTGTTCGCATTGGAGGCCGT |
| | | AGTCATATCTGTTGGATCCATGGGACTTATTATCTCCCTGTTGTGTGTGTA |
| | | CTTCTGGCTGGAACGGACTATGCCCAGGATCCCCACGCTCAAGAATCTGG |
| | | AAGATCTCGTCACAGAATACCATGGTAATTTCAGCGCCTGGAGCGGAGTC |
| | | TCTAAGGGTCTGGCCGAATCCCTCCAACCCGATTATTCTGAACGGTTGTG |
| | | CCTCGTATCCGAAATACCACCAAAAGGCGGGGCTCTGGGTGAGGGCCCA |
| | | GGGGCGAGTCCGTGCAATCAACACAGCCCGTATTGGGCCCCTCCTTGTTA |
| | | TACGTTGAAGCCCGAAACT |
| 83 | CISCγ: FKBP- | MPLGLLWLGLALLGALHAQAGVQVETISPGDGRTFPKRGQTCVVHYTGMLE |
| | IL2Rγ amino | DGKKFDSSRDRNKPFKFMLGKQEVIRGWEEGVAQMSVGQRAKLTISPDYAY |
| | acid sequence | GATGHPGIIPPHATLVFDVELLKLGEGGSPGSNTSKENPFLFALEAVVISVGS |
| | | MGLIISLLCVYFWLERTMPRIPTLKNLEDLVTEYHGNFSAWSGVSKGLAESL |
| | | QPDYSERLCLVSEIPPKGGALGEGPGASPCNQHSPYWAPPCYTLKPET |
| 84 | | (left blank) |
| 85 | DISC: CISC- | MPLGLLWLGLALLGALHAQAGVQVETISPGDGRTFPKRGQTCVVHYTGMLE |
| | FRB; μDISC | DGKKFDSSRDRNKPFKFMLGKQEVIRGWEEGVAQMSVGQRAKLTISPDYAY |
| | amino acid | GATGHPGIIPPHATLVFDVELLKLGEGGSPGSNTSKENPFLFALEAVVISVGS |
| | sequence | MGLIISLLCVYFWLERTMPRIPTLKNLEDLVTEYHGNFSAWSGVSKGLAESL |
| | | QPDYSERLCLVSEIPPKGGALGEGPGASPCNQHSPYWAPPCYTLKPETGSGAT |
| | | NFSLLKQAGDVEENPGPMALPVTALLLPLALLLHAARPILWHEMWHEGLEE |
| | | ASRLYFGERNVKGMFEVLEPLHAMMERGPQTLKETSFNQAYGRDLMEAQE |
| | | WCRKYMKSGNVKDLLQAWDLYYHVFRRISKPAALGKDTIPWLGHLLVGLS |
| | | GAFGFIILVYLLINCRNTGPWLKKVLKCNTPDPSKFFSQLSSEHGGDVQKWLS |
| | | SPFPSSSFSPGGLAPEISPLEVLERDKVTQLLLQQDKVPEPASLSSNHSLTSCFT |
| | | NQGYFFFHLPDALEIEACQVYFTYDPYSEEDPDEGVAGAPTGSSPQPLQPLSG |
| | | EDDAYCTFPSRDDLLLFSPSLLGGPSPPSTAPGGSGAGEERMPPSLQERVPRD |
| | | WDPQPLGPPTPGVPDLVDFQPPPELVLREAGEEVPDAGPREGVSFPWSRPPG |
| | | QGEFRALNARLPLNTDAYLSLQELQGQDPTHLVGSGATNFSLLKQAGDVEE |
| | | NPGPEMWHEGLEEASRLYFGERNVKGMFEVLEPLHAMMERGPQTLKETSFN |
| | | QAYGRDLMEAQEWCRKYMKSGNVKDLLQAWDLYYHVFRRISK |
| 86 | FRB: express | GAGATGTGGCATGAGGGTCTGGAAGAAGCGTCTCGACTGTACTTTGGTGA |
| | intracellularly to | GCGCAATGTGAAGGGCATGTTTGAAGTCCTCGAACCCCTTCATGCCATGA |
| | function as a | TGGAACGCGGACCCCAGACCTTGAAGGAGACAAGTTTTAACCAAGCTTA |
| | decoy for | CGGAAGAGACCTGATGGAAGCCCAGGAATGGTGCAGGAAATACATGAAA |
| | rapamycin: | AGCGGGAATGTGAAGGACTTGACCCAAGCGTGGGACCTGTACTATCATGT |
| | FRB; nucleotide | CTTTAGGCGCATTAGTAAG |
| | sequence | |
| 87 | FRB amino acid | EMWHEGLEEASRLYFGERNVKGMFEVLEPLHAMMERGPQTLKETSFNQAY |
| | sequence | GRDLMEAQEWCRKYMKSGNVKDLTQAWDLYYHVFRRISK |
| 88 | LNGFR coding | ATGGGGGCAGGTGCCACCGGACGAGCCATGGACGGGCCGCGCCTGCTGC |
| | sequence with | TGTTGCTGCTTCTGGGGGTGTCCCTTGGAGGTGCCAAGGAGGCATGCCCC |
| | stop codon | ACAGGCCTGTACACACACAGCGGTGAGTGCTGCAAAGCCTGCAACCTGG |
| | | GCGAGGGTGTGGCCCAGCCTTGTGGAGCCAACCAGACCGTGTGTGAGCC |
| | | CTGCCTGGACAGCGTGACGTTCTCCGACGTGGTGAGCGCGACCGAGCCGT |
| | | GCAAGCCGTGCACCGAGTGCGTGGGGCTCCAGAGCATGTCGGCGCCGTG |
| | | CGTGGAGGCCGACGACGCCGTGTGCCGCTGCGCCTACGGCTACTACCAGG |
| | | ATGAGACGACTGGGCGCTGCGAGGCGTGCCGCGTGTGCGAGGCGGGCTC |
| | | GGGCCTCGTGTTCTCCTGCCAGGACAAGCAGAACACCGTGTGCGAGGAGT |
| | | GCCCCGACGGCACGTATTCCGACGAGGCCAACCACGTGGACCCGTGCCTG |
| | | CCCTGCACCGTGTGCGAGGACACCGAGCGCCAGCTCCGCGAGTGCACAC |
| | | GCTGGGCCGACGCCGAGTGCGAGGAGATCCCTGGCCGTTGGATTACACG |
| | | GTCCACACCCCCAGAGGGCTCGGACAGCACAGCCCCCAGCACCCAGGAG |
| | | CCTGAGGCACCTCCAGAACAAGACCTCATAGCCAGCACGGTGGCAGGTG |
| | | TGGTGACCACAGTGATGGGCAGCTCCCAGCCCGTGGTGACCCGAGGCAC |
| | | CACCGACAACCTCATCCCTGTCTATTGCTCCATCCTGGCTGCTGTGGTTGT |
| | | GGGTCTTGTGGCCTACATAGCCTTCAAGAGGTGA |
| 89 | P2A self- | GGAAGCGGAGCGACTAACTTCAGCCTGCTGAAGCAGGCCGGAGATGTGG |
| | cleaving peptide | AGGAAAACCCTGGACCG |
| 90 | 0.25 kb human | TGCTAGCGTGGGCAGGCAAGCCAGGTGCTGGACCTCTGCACGTGGGGCA |
| | FOXP3 5′HA | TGTGTGGGTATGTACATGTACCTGTGTTCTTGGTGTGTGTGTGTGTGTGTG |
| | designed for | TGTGTGTGTGTGTCTAGAGCTGGGGTGCAACTATGGGGCCCCTCGGGACA |
| | both TALEN | TGTCCCAGCCAATGCCTGCTTTGACCAGAGGAGTGTCCACGTGGCTCAGG |
| | and Cas9 | TGGTCGAGTATCTCATACCGCCCTAGCACACGTGTGACTCCTTTCCCCTAT |
| | approaches | TGTCTAC |
| 91 | 0.3 kb human | CATGTGTGGGTATGTACATGTACCTGTGTTCTTGGTGTGTGTGTGTGTGTG |
| | FOXP3 5′HA for | TGTGTGTGTGTGTGTCTAGAGCTGGGGTGCAACTATGGGGCCCCTCGGGA |
| | Cas9-T9 | CATGTCCCAGCCAATGCCTGCTTTGACCAGAGGAGTGTCCACGTGGCTCA |
| | | GGTGGTCGAGTATCTCATACCGCCCTAGCACACGTGTGACTCCTTTCCCCT |
| | | ATTGTCTACGCAGCCTGCCCTTGGACAAGGACCCGATGCCCAACCCCAGG |
| | | CCTGGCAAGCCCTCGGCCCCTTCCTTGGCCCTTGGCCCATCCCC |
| 92 | 0.45 kb human | AGCCTGTGCAGGGTGCAGGGAGGGCTAGAGGCCTGAGGCTTGAAACAGC |
| | FOXP3 5′HA for | TCTCAAGTGGAGGGGGAAACAACCATTGCCCTCATAGAGGACACATCCA |
| | Cas9-T9 | CACCAGGGCTGTGCTAGCGTGGGCAGGCAAGCCAGGTGCTGGACCTCTG |
| | | CACGTGGGGCATGTGTGGGTATGTACATGTACCTGTGTTCTTGGTGTGTGT |
| | | GTGTGTGTGTGTGTGTGTGTGTGTCTAGAGCTGGGGTGCAACTATGGGGC |
| | | CCCTCGGGACATGTCCCAGCCAATGCCTGCTTTGACCAGAGGAGTGTCCA |
| | | CGTGGCTCAGGTGGTCGAGTATCTCATACCGCCCTAGCACACGTGTGACT |
| | | CCTTTCCCCTATTGTCTACGCAGCCTGCCCTTGGACAAGGACCCGATGCCC |
| | | AACCCCAGGCCTGGCAAGCCCTCGGCCCCTTCCTTGGCCCTTGGCCCATC |
| | | CCC |
| 93 | 0.6 kb human | ATCACTTGCCAGGACTGTTACAATAGCCTCCTCACTAGCCCCACTCACAG |
| | FOXP3 5′HA for | CAGCCAGATGAATCTTTTGAGTCCATGCCTAGTCACTGGGGCAAAATAGG |
| | Cas9-T9 | ACTCCGAGGAGAAAGTCCGAGACCAGCTCCGGCAAGATGAGCAAACACA |
| | | GCCTGTGCAGGGTGCAGGGAGGGCTAGAGGCCTGAGGCTTGAAACAGCT |
| | | CTCAAGTGGAGGGGGAAACAACCATTGCCCTCATAGAGGACACATCCAC |
| | | ACCAGGGCTGTGCTAGCGTGGGCAGGCAAGCCAGGTGCTGGACCTCTGC |
| | | ACGTGGGGCATGTGTGGGTATGTACATGTACCTGTGTTCTTGGTGTGTGT |
| | | GTGTGTGTGTGTGTGTGTGTGTGTCTAGAGCTGGGGTGCAACTATGGGGC |
| | | CCCTCGGGACATGTCCCAGCCAATGCCTGCTTTGACCAGAGGAGTGTCCA |
| | | CGTGGCTCAGGTGGTCGAGTATCTCATACCGCCCTAGCACACGTGTGACT |
| | | CCTTTCCCCTATTGTCTACGCAGCCTGCCCTTGGACAAGGACCCGATGCCC |
| | | AACCCCAGGCCTGGCAAGCCCTCGGCCCCTTCCTTGGCCCTTGGCCCATC |
| | | CCC |
| 94 | 0.8 kb human | ATCTCAGGTAATGTCAGCTCGGTCCTTCCAGCTGCTCAAGCTAAAACCCA |
| | FOXP3 5′HA for | TGTCACTTTGACTCTCCCTCTTGCCCACTACATCCAAGCTGCTAGCACTGC |
| | Cas9-T9 | TCCTGATCCAGCTTCAGATTAAGTCTCAGAATCTACCCACTTCTCGCCTTC |
| | | TCCACTGCCACCAGCCCATTCTGTGCCAGCATCATCACTTGCCAGGACTG |
| | | TTACAATAGCCTCCTCACTAGCCCCACTCACAGCAGCCAGATGAATCTTT |
| | | TGAGTCCATGCCTAGTCACTGGGGCAAAATAGGACTCCGAGGAGAAAGT |
| | | CCGAGACCAGCTCCGGCAAGATGAGCAAACACAGCCTGTGCAGGGTGCA |
| | | GGGAGGGCTAGAGGCCTGAGGCTTGAAACAGCTCTCAAGTGGAGGGGGA |
| | | AACAACCATTGCCCTCATAGAGGACACATCCACACCAGGGCTGTGCTAGC |
| | | GTGGGCAGGCAAGCCAGGTGCTGGACCTCTGCACGTGGGGCATGTGTGG |
| | | GTATGTACATGTACCTGTGTTCTTGGTGTGTGTGTGTGTGTGTGTGTGTGT |
| | | GTGTGTCTAGAGCTGGGGTGCAACTATGGGGCCCCTCGGGACATGTCCCA |
| | | GCCAATGCCTGCTTTGACCAGAGGAGTGTCCACGTGGCTCAGGTGGTCGA |
| | | GTATCTCATACCGCCCTAGCACACGTGTGACTCCTTTCCCCTATTGTCTAC |
| | | GCAGCCTGCCCTTGGACAAGGACCCGATGCCCAACCCCAGGCCTGGCAA |
| | | GCCCTCGGCCCCTTCCTTGGCCCTTGGCCCATCCCC |
| 95 | 0.3 kb human | GACATGTCCCAGCCAATGCCTGCTTTGACCAGAGGAGTGTCCACGTGGCT |
| | FOXP3 5′HA for | CAGGTGGTCGAGTATCTCATACCGCCCTAGCACACGTGTGACTCCTTTCC |
| | Cas9-T3 (actual | CCTATTGTCTACGCAGCCTGCCCTTGGACAAGGACCCGATGCCCAACCCC |
| | length 0.275 kb) | AGGCCTGGCAAGCCCTCGGCCCCTTCCTTGGCCCTTGGCCCATCCCCAGG |
| | | AGCCTCGCCCAGCTGGAGGGCTGCACCCAAAGCCTCAGACCTGCTGGGG |
| | | GCCCGGGGCCCAGGGGGAACCTTCCA |
| 96 | 0.45 kb human | CATAGAGGACACATCCACACCAGGGCTGTGCTAGCGTGGGCAGGCAAGC |
| | FOXP3 5′HA for | CAGGTGCTGGACCTCTGCACGTGGGGCATGTGTGGGTATGTACATGTACC |
| | Cas9-T3 | TGTGTTCTTGGTGTGTGTGTGTGTGTGTGTGTGTGTGTGTGTCTAGAGCTG |
| | | GGGTGCAACTATGGGGCCCCTCGGGACATGTCCCAGCCAATGCCTGCTTT |
| | | GACCAGAGGAGTGTCCACGTGGCTCAGGTGGTCGAGTATCTCATACCGCC |
| | | CTAGCACACGTGTGACTCCTTTCCCCTATTGTCTACGCAGCCTGCCCTTGG |
| | | ACAAGGACCCGATGCCCAACCCCAGGCCTGGCAAGCCCTCGGCCCCTTCC |
| | | TTGGCCCTTGGCCCATCCCCAGGAGCCTCGCCCAGCTGGAGGGCTGCACC |
| | | CAAAGCCTCAGACCTGCTGGGGGCCCGGGGCCCAGGGGGAACCTTCCA |
| 97 | 0.6 kb human | CTAGTCACTGGGGCAAAATAGGACTCCGAGGAGAAAGTCCGAGACCAGC |
| | FOXP3 5′HA for | TCCGGCAAGATGAGCAAACACAGCCTGTGCAGGGTGCAGGGAGGGCTAG |
| | Cas9-T3 | AGGCCTGAGGCTTGAAACAGCTCTCAAGTGGAGGGGGAAACAACCATTG |
| | | CCCTCATAGAGGACACATCCACACCAGGGCTGTGCTAGCGTGGGCAGGC |
| | | AAGCCAGGTGCTGGACCTCTGCACGTGGGGCATGTGTGGGTATGTACATG |
| | | TACCTGTGTTCTTGGTGTGTGTGTGTGTGTGTGTGTGTGTGTGTGTCTAGA |
| | | GCTGGGGTGCAACTATGGGGCCCCTCGGGACATGTCCCAGCCAATGCCTG |
| | | CTTTGACCAGAGGAGTGTCCACGTGGCTCAGGTGGTCGAGTATCTCATAC |
| | | CGCCCTAGCACACGTGTGACTCCTTTCCCCTATTGTCTACGCAGCCTGCCC |
| | | TTGGACAAGGACCCGATGCCCAACCCCAGGCCTGGCAAGCCCTCGGCCCC |
| | | TTCCTTGGCCCTTGGCCCATCCCCAGGAGCCTCGCCCAGCTGGAGGGCTG |
| | | CACCCAAAGCCTCAGACCTGCTGGGGGCCCGGGGCCCAGGGGGAACCTT |
| | | CCA |
| 98 | 0.25 kb human | GTGAGGCCCTGGGCCCAGGATGGGGCAGGCAGGGTGGGGTACCTGGACC |
| | FOXP3 3′HA | TACAGGTGCCGACCTTTACTGTGGCACTGGGCGGGAGGGGGGCTGGCTG |
| | designed for | GGGCACAGGAAGTGGTTTCTGGGTCCCAGGCAAGTCTGTGACTTATGCAG |
| | both TALEN | ATGTTGCAGGGCCAAGAAAATCCCCACCTGCCAGGCCTCAGAGATTGGA |
| | and Cas9 | GGCTCTCCCCGACCTCCCAATCCCTGTCTCAGGAGAGGAGGAGGCCGT |
| | approaches | |
| 99 | 0.3 kb human | GCCTCGCCCAGCTGGAGGGCTGCACCCAAAGCCTCAGACCTGCTGGGGG |
| | FOXP3 3′HA for | CCCGGGGCCCAGGGGGAACCTTCCAGGGCCGAGATCTTCGAGGCGGGGC |
| | Cas9-T9 | CCATGCCTCCTCTTCTTCCTTGAACCCCATGCCACCATCGCAGCTGCAGGT |
| | | GAGGCCCTGGGCCCAGGATGGGGCAGGCAGGGTGGGGTACCTGGACCTA |
| | | CAGGTGCCGACCTTTACTGTGGCACTGGGCGGGAGGGGGGCTGGCTGGG |
| | | GCACAGGAAGTGGTTTCTGGGTCCCAGGCAAGTCTGTGACTTATGCAGAT |
| | | GTT |
| 100 | 0.45 kb human | GCCTCGCCCAGCTGGAGGGCTGCACCCAAAGCCTCAGACCTGCTGGGGG |
| | FOXP3 3′HA for | CCCGGGGCCCAGGGGGAACCTTCCAGGGCCGAGATCTTCGAGGCGGGGC |
| | Cas9-T9 | CCATGCCTCCTCTTCTTCCTTGAACCCCATGCCACCATCGCAGCTGCAGGT |
| | | GAGGCCCTGGGCCCAGGATGGGGCAGGCAGGGTGGGGTACCTGGACCTA |
| | | CAGGTGCCGACCTTTACTGTGGCACTGGGCGGGAGGGGGGCTGGCTGGG |
| | | GCACAGGAAGTGGTTTCTGGGTCCCAGGCAAGTCTGTGACTTATGCAGAT |
| | | GTTGCAGGGCCAAGAAAATCCCCACCTGCCAGGCCTCAGAGATTGGAGG |
| | | CTCTCCCCGACCTCCCAATCCCTGTCTCAGGAGAGGAGGAGGCCGTATTG |
| | | TAGTCCCATGAGCATAGCTATGTGTCCCCATCCCCATGTGACAAGAGAAG |
| | | AGGA |
| 101 | 0.6 kb human | GCCTCGCCCAGCTGGAGGGCTGCACCCAAAGCCTCAGACCTGCTGGGGG |
| | FOXP3 3′HA for | CCCGGGGCCCAGGGGGAACCTTCCAGGGCCGAGATCTTCGAGGCGGGGC |
| | Cas9-T9 | CCATGCCTCCTCTTCTTCCTTGAACCCCATGCCACCATCGCAGCTGCAGGT |
| | | GAGGCCCTGGGCCCAGGATGGGGCAGGCAGGGTGGGGTACCTGGACCTA |
| | | CAGGTGCCGACCTTTACTGTGGCACTGGGCGGGAGGGGGGCTGGCTGGG |
| | | GCACAGGAAGTGGTTTCTGGGTCCCAGGCAAGTCTGTGACTTATGCAGAT |
| | | GTTGCAGGGCCAAGAAAATCCCCACCTGCCAGGCCTCAGAGATTGGAGG |
| | | CTCTCCCCGACCTCCCAATCCCTGTCTCAGGAGAGGAGGAGGCCGTATTG |
| | | TAGTCCCATGAGCATAGCTATGTGTCCCCATCCCCATGTGACAAGAGAAG |
| | | AGGACTGGGGCCAAGTAGGTGAGGTGACAGGGCTGAGGCCAGCTCTGCA |
| | | ACTTATTAGCTGTTTGATCTTTAAAAAGTTACTCGATCTCCATGAGCCTCA |
| | | GTTTCCATACGTGTAAAAGGGGGATGATCATAGCATCTACCATGTGGGCT |
| | | TGCA |
| 102 | 0.8 kb human | GCCTCGCCCAGCTGGAGGGCTGCACCCAAAGCCTCAGACCTGCTGGGGG |
| | FOXP3 3′HA for | CCCGGGGCCCAGGGGGAACCTTCCAGGGCCGAGATCTTCGAGGCGGGGC |
| | Cas9-T9 | CCATGCCTCCTCTTCTTCCTTGAACCCCATGCCACCATCGCAGCTGCAGGT |
| | | GAGGCCCTGGGCCCAGGATGGGGCAGGCAGGGTGGGGTACCTGGACCTA |
| | | CAGGTGCCGACCTTTACTGTGGCACTGGGCGGGAGGGGGGCTGGCTGGG |
| | | GCACAGGAAGTGGTTTCTGGGTCCCAGGCAAGTCTGTGACTTATGCAGAT |
| | | GTTGCAGGGCCAAGAAAATCCCCACCTGCCAGGCCTCAGAGATTGGAGG |
| | | CTCTCCCCGACCTCCCAATCCCTGTCTCAGGAGAGGAGGAGGCCGTATTG |
| | | TAGTCCCATGAGCATAGCTATGTGTCCCCATCCCCATGTGACAAGAGAAG |
| | | AGGACTGGGGCCAAGTAGGTGAGGTGACAGGGCTGAGGCCAGCTCTGCA |
| | | ACTTATTAGCTGTTTGATCTTTAAAAAGTTACTCGATCTCCATGAGCCTCA |
| | | GTTTCCATACGTGTAAAAGGGGGATGATCATAGCATCTACCATGTGGGCT |
| | | TGCAGTGCAGAGTATTTGAATTAGACACAGAACAGTGAGGATCAGGATG |
| | | GCCTCTCACCCACCTGCCTTTCTGCCCAGCTGCCCACACTGCCCCTAGTCA |
| | | TGGTGGCACCCTCCGGGGCACGGCTGGGCCCCTTGCCCCACTTACAGGCA |
| | | CTCCTCCAGGACAGGCCACATTTCATGCACCAGGTATGGACGGTGAAT |
| 103 | 0.3 kb human | CGAGATCTTCGAGGCGGGGCCCATGCCTCCTCTTCTTCCTTGAACCCCATG |
| | FOXP3 3′HA for | CCACCATCGCAGCTGCAGGTGAGGCCCTGGGCCCAGGATGGGGCAGGCA |
| | Cas9-T3 | GGGTGGGGTACCTGGACCTACAGGTGCCGACCTTTACTGTGGCACTGGGC |
| | | GGGAGGGGGGCTGGCTGGGGCACAGGAAGTGGTTTCTGGGTCCCAGGCA |
| | | AGTCTGTGACTTATGCAGATGTTGCAGGGCCAAGAAAATCCCCACCTGCC |
| | | AGGCCTCAGAGATTGGAGGCTCTCCCCGACCTCCCAATCCCTGTCTCAGG |
| | | A |
| 104 | 0.45 kb human | CGAGATCTTCGAGGCGGGGCCCATGCCTCCTCTTCTTCCTTGAACCCCATG |
| | FOXP3 3′HA for | CCACCATCGCAGCTGCAGGTGAGGCCCTGGGCCCAGGATGGGGCAGGCA |
| | Cas9-T3 | GGGTGGGGTACCTGGACCTACAGGTGCCGACCTTTACTGTGGCACTGGGC |
| | | GGGAGGGGGGCTGGCTGGGGCACAGGAAGTGGTTTCTGGGTCCCAGGCA |
| | | AGTCTGTGACTTATGCAGATGTTGCAGGGCCAAGAAAATCCCCACCTGCC |
| | | AGGCCTCAGAGATTGGAGGCTCTCCCCGACCTCCCAATCCCTGTCTCAGG |
| | | AGAGGAGGAGGCCGTATTGTAGTCCCATGAGCATAGCTATGTGTCCCCAT |
| | | CCCCATGTGACAAGAGAAGAGGACTGGGGCCAAGTAGGTGAGGTGACAG |
| | | GGCTGAGGCCAGCTCTGCAACTTATTAGCTGTTTGATCTTTAAAAAGTTA |
| | | CTC |
| 105 | 0.6 kb human | CGAGATCTTCGAGGCGGGGCCCATGCCTCCTCTTCTTCCTTGAACCCCATG |
| | FOXP3 3′HA for | CCACCATCGCAGCTGCAGGTGAGGCCCTGGGCCCAGGATGGGGCAGGCA |
| | Cas9-T3 | GGGTGGGGTACCTGGACCTACAGGTGCCGACCTTTACTGTGGCACTGGGC |
| | | GGGAGGGGGGCTGGCTGGGGCACAGGAAGTGGTTTCTGGGTCCCAGGCA |
| | | AGTCTGTGACTTATGCAGATGTTGCAGGGCCAAGAAAATCCCCACCTGCC |
| | | AGGCCTCAGAGATTGGAGGCTCTCCCCGACCTCCCAATCCCTGTCTCAGG |
| | | AGAGGAGGAGGCCGTATTGTAGTCCCATGAGCATAGCTATGTGTCCCCAT |
| | | CCCCATGTGACAAGAGAAGAGGACTGGGGCCAAGTAGGTGAGGTGACAG |
| | | GGCTGAGGCCAGCTCTGCAACTTATTAGCTGTTTGATCTTTAAAAAGTTA |
| | | CTCGATCTCCATGAGCCTCAGTTTCCATACGTGTAAAAGGGGGATGATCA |
| | | TAGCATCTACCATGTGGGCTTGCAGTGCAGAGTATTTGAATTAGACACAG |
| | | AACAGTGAGGATCAGGATGGCCTCTCACCCACCTGCCTTTCTGCCCAGCT |
| | | GC |
| 106 | 0.25 kb AAVS1 | TAGCCACCTCTCCATCCTCTTGCTTTCTTTGCCTGGACACCCCGTTCTCCTG |
| | 5′HA for Cas9- | TGGATTCGGGTCACCTCTCACTCCTTTCATTTGGGCAGCTCCCCTACCCCC |
| | P1 and Cas9-N2 | CTTACCTCTCTAGTCTGTGCTAGCTCTTCCAGCCCCCTGTCATGGCATCTT |
| | | CCAGGGGTCCGAGAGCTCAGCTAGTCTTCTTCCTCCAACCCGGGCCCCTA |
| | | TGTCCACTTCAGGACAGCATGTTTGCTGCCTCCAGGGATCCTGTGT |
| 107 | 0.6 kb AAVS1 | AGGTTCCGTCTTCCTCCACTCCCTCTTCCCCTTGCTCTCTGCTGTGTTGCTG |
| | 5′HA for Cas9- | CCCAAGGATGCTCTTTCCGGAGCACTTCCTTCTCGGCGCTGCACCACGTG |
| | P1 and Cas9-N2 | ATGTCCTCTGAGCGGATCCTCCCCGTGTCTGGGTCCTCTCCGGGCATCTCT |
| | | CCTCCCTCACCCAACCCCATGCCGTCTTCACTCGCTGGGTTCCCTTTTCCT |
| | | TCTCCTTCTGGGGCCTGTGCCATCTCTCGTTTCTTAGGATGGCCTTCTCCG |
| | | ACGGATGTCTCCCTTGCGTCCCGCCTCCCCTTCTTGTAGGCCTGCATCATC |
| | | ACCGTTTTTCTGGACAACCCCAAAGTACCCCGTCTCCCTGGCTTTAGCCAC |
| | | CTCTCCATCCTCTTGCTTTCTTTGCCTGGACACCCCGTTCTCCTGTGGATTC |
| | | GGGTCACCTCTCACTCCTTTCATTTGGGCAGCTCCCCTACCCCCCTTACCT |
| | | CTCTAGTCTGTGCTAGCTCTTCCAGCCCCCTGTCATGGCATCTTCCAGGGG |
| | | TCCGAGAGCTCAGCTAGTCTTCTTCCTCCAACCCGGGCCCCTATGTCCACT |
| | | TCAGGACAGCATGTTTGCTGCCTCCAGGGATCCTGTGT |
| 108 | 0.25 kb AAVS1 | CTCTGGTTCTGGGTACTTTTATCTGTCCCCTCCACCCCACAGTGGGGCCAC |
| | 3′HA for Cas9- | TAGGGACAGGATTGGTGACAGAAAAGCCCCATCCTTAGGCCTCCTCCTTC |
| | P1 and Cas9-N2 | CTAGTCTCCTGATATTGGGTCTAACCCCCACCTCCTGTTAGGCAGATTCCT |
| | | TATCTGGTGACACACCCCCATTTCCTGGAGCCATCTCTCTCCTTGCCAGAA |
| | | CCTCTAAGGTTTGCTTACGATGGAGCCAGAGAGGATCCTGGGAGGGA |
| 109 | 0.6 kb AAVS1 | CTCTGGTTCTGGGTACTTTTATCTGTCCCCTCCACCCCACAGTGGGGCCAC |
| | 3′HA for Cas9- | TAGGGACAGGATTGGTGACAGAAAAGCCCCATCCTTAGGCCTCCTCCTTC |
| | P1 and Cas9-N2 | CTAGTCTCCTGATATTGGGTCTAACCCCCACCTCCTGTTAGGCAGATTCCT |
| | | TATCTGGTGACACACCCCCATTTCCTGGAGCCATCTCTCTCCTTGCCAGAA |
| | | CCTCTAAGGTTTGCTTACGATGGAGCCAGAGAGGATCCTGGGAGGGAGA |
| | | GCTTGGCAGGGGGTGGGAGGGAAGGGGGGGATGCGTGACCTGCCCGGTT |
| | | CTCAGTGGCCACCCTGCGCTACCCTCTCCCAGAACCTGAGCTGCTCTGAC |
| | | GCGGCCGTCTGGTGCGTTTCACTGATCCTGGTGCTGCAGCTTCCTTACACT |
| | | TCCCAAGAGGAGAAGCAGTTTGGAAAAACAAAATCAGAATAAGTTGGTC |
| | | CTGAGTTCTAACTTTGGCTCTTCACCTTTCTAGTCCCCAATTTATATTGTTC |
| | | CTCCGTGCGTCAGTTTTACCTGTGAGATAAGGCCAGTAGCCAGCCCCGTC |
| | | CTGGCAGGGCTGTGGTGAGGAGGGGGGTGTCCGTGTGGAAAACTCCC |
| 110 | LNGFRt protein | MGAGATGRAMDGPRLLLLLLLGVSLGGAKEACPTGLYTHSGECCKACNLG |
| | sequence | EGVAQPCGANQTVCEPCLDSVTFSDVVSATEPCKPCTECVGLQSMSAPCVEA |
| | | DDAVCRCAYGYYQDETTGRCEACRVCEAGSGLVFSCQDKQNTVCEECPDG |
| | | TYSDEANHVDPCLPCTVCEDTERQLRECTRWADAECEEIPGRWITRSTPPEGS |
| | | DSTAPSTQEPEAPPEQDLIASTVAGVVTTVMGSSQPVVTRGTTDNLIPVYCSIL |
| | | AAVVVGLVAYIAFKR |
| 111 | RQR8 protein | MGTSLLCWMALCLLGADHADACPYSNPSLCSGGGGSELPTQGTFSNVSTNV |
| | sequence | SPAKPTTTACPYSNPSLCSGGGGSPAPRPPTPAPTIASQPLSLRPEACRPAAGG |
| | | AVHTRGLDFACDIYIWAPLAGTCGVLLLSLVITLYCNHRNRRRVCKCPRPVV |
| 112 | EGFRt with | MLLLVTSLLLCELPHPAFLLIPRKVCNGIGIGEFKDSLSINATNIKHFKNCTSIS |
| | GM-CSFR | GDLHILPVAFRGDSFTHTPPLDPQELDILKTVKEITGFLLIQAWPENRTDLHAF |
| | signal peptide | ENLEIIRGRTKQHGQFSLAVVSLNITSLGLRSLKEISDGDVIISGNKNLCYANTI |
| | | NWKKLFGTSGQKTKIISNRGENSCKATGQVCHALCSPEGCWGPEPRDCVSCR |
| | | NVSRGRECVDKCNLLEGEPREFVENSECIQCHPECLPQAMNITCTGRGPDNCI |
| | | QCAHYIDGPHCVKTCPAGVMGENNTLVWKYADAGHVCHLCHPNCTYGCT |
| | | GPGLEGCPTNGPKIPSIATGMVGALLLLLVVALGIGLFM |
| 113 | MND promoter | GAACAGAGAAACAGGAGAATATGGGCCAAACAGGATATCTGTGGTAAGC |
| | | AGTTCCTGCCCCGGCTCAGGGCCAAGAACAGTTGGAACAGCAGAATATG |
| | | GGCCAAACAGGATATCTGTGGTAAGCAGTTCCTGCCCCGGCTCAGGGCCA |
| | | AGAACAGATGGTCCCCAGATGCGGTCCCGCCCTCAGCAGTTTCTAGAGAA |
| | | CCATCAGATGTTTCCAGGGTGCCCCAAGGACCTGAAATGACCCTGTGCCT |
| | | TATTTGAACTAACCAATCAGTTCGCTTCTCGCTTCTGTTCGCGCGCTTCTG |
| | | CTCCCCGAGCTCTATATAAGCAGAGCTCGTTTAGTGAACCGTCAGATC |
| 114 | PGK promoter | CCACGGGGTTGGGGTTGCGCCTTTTCCAAGGCAGCCCTGGGTTTGCGCAG |
| | | GGACGCGGCTGCTCTGGGCGTGGTTCCGGGAAACGCAGCGGCGCCGACC |
| | | CTGGGTCTCGCACATTCTTCACGTCCGTTCGCAGCGTCACCCGGATCTTCG |
| | | CCGCTACCCTTGTGGGCCCCCCGGCGACGCTTCCTGCTCCGCCCCTAAGTC |
| | | GGGAAGGTTCCTTGCGGTTCGCGGCGTGCCGGACGTGACAAACGGAAGC |
| | | CGCACGTCTCACTAGTACCCTCGCAGACGGACAGCGCCAGGGAGCAATG |
| | | GCAGCGCGCCGACCGCGATGGGCTGTGGCCAATAGCGGCTGCTCAGCGG |
| | | GGCGCGCCGAGAGCAGCGGCCGGGAAGGGGCGGTGCGGGAGGCGGGGT |
| | | GTGGGGCGGTAGTGTGGGCCCTGTTCCTGCCCGCGCGGTGTTCCGCATTC |
| | | TGCAAGCCTCCGGAGCGCACGTCGGCAGTCGGCTCCCTCGTTGACCGAAT |
| | | CACCGACCTCTCTCCCCAGGGGGATCC |
| 115 | EF1 promoter | AGGCTCCGGTGCCCGTCAGTGGGCAGAGCGCACATCGCCCACAGTCCCCG |
| | | AGAAGTTGGGGGGAGGGGTCGGCAATTGAACCGGTGCCTAGAGAAGGTG |
| | | GCGCGGGGTAAACTGGGAAAGTGATGTCGTGTACTGGCTCCGCCTTTTTC |
| | | CCGAGGGTGGGGGAGAACCGTATATAAGTGCAGTAGTCGCCGTGAACGT |
| | | TCTTTTTCGCAACGGGTTTGCCGCCAGAACACA |
| 116 | SV40 polyA | TGCTTTATTTGTGAAATTTGTGATGCTATTGCTTTATTTGTAACCATTATA |
| | | AGCTGCAATAAACAAGTTAACAACAACAATTGCATTCATTTTATGTTTCA |
| | | GGTTCAGGGGGAGATGTGGGAGGTTTTTTAAAGC |
| 117 | 3′UTR of | CCTCAAGATCAAGGAAAGGAGGATGGACGAACAGGGGCCAAACTGGTGG |
| | FOXP3 | GAGGCAGAGGTGGTGGGGGCAGGGATGATAGGCCCTGGATGTGCCCACA |
| | | GGGACCAAGAAGTGAGGTTTCCACTGTCTTGCCTGCCAGGGCCCCTGTTC |
| | | CCCCGCTGGCAGCCACCCCCTCCCCCATCATATCCTTTGCCCCAAGGCTGC |
| | | TCAGAGGGGCCCCGGTCCTGGCCCCAGCCCCCACCTCCGCCCCAGACACA |
| | | CCCCCCAGTCGAGCCCTGCAGCCAAACAGAGCCTTCACAACCAGCCACAC |
| | | AGAGCCTGCCTCAGCTGCTCGCACAGATTACTTCAGGGCTGGAAAAGTCA |
| | | CACAGACACACAAAATGTCACAATCCTGTCCCTCACTCAACACAAACCCC |
| | | AAAACACAGAGAGCCTGCCTCAGTACACTCAAACAACCTCAAAGCTGCA |
| | | TCATCACACAATCACACACAAGCACAGCCCTGACAACCCACACACCCCA |
| | | AGGCACGCACCCACAGCCAGCCTCAGGGCCCACAGGGGCACTGTCAACA |
| | | CAGGGGTGTGCCCAGAGGCCTACACAGAAGCAGCGTCAGTACCCTCAGG |
| | | ATCTGAGGTCCCAACACGTGCTCGCTCACACACACGGCCTGTTAGAATTC |
| | | ACCTGTGTATCTCACGCATATGCACACGCACAGCCCCCCAGTGGGTCTCT |
| | | TGAGTCCCGTGCAGACACACACAGCCACACACACTGCCTTGCCAAAAATA |
| | | CCCCGTGTCTCCCCTGCCACTCACCTCACTCCCATTCCCTGAGCCCTGATC |
| | | CATGCCTCAGCTTAGACTGCAGAGGAACTACTCATTTATTTGGGATCCAA |
| | | GGCCCCCAACCCACAGTACCGTCCCCAATAAACTGCAGCCGAGCTCCCCA |
| | | CA |
| 118 | LNGFR coding | ATGGGGGCAGGTGCCACCGGACGAGCCATGGACGGGCCGCGCCTGCTGC |
| | sequence | TGTTGCTGCTTCTGGGGGTGTCCCTTGGAGGTGCCAAGGAGGCATGCCCC |
| | without stop | ACAGGCCTGTACACACACAGCGGTGAGTGCTGCAAAGCCTGCAACCTGG |
| | codon | GCGAGGGTGTGGCCCAGCCTTGTGGAGCCAACCAGACCGTGTGTGAGCC |
| | | CTGCCTGGACAGCGTGACGTTCTCCGACGTGGTGAGCGCGACCGAGCCGT |
| | | GCAAGCCGTGCACCGAGTGCGTGGGGCTCCAGAGCATGTCGGCGCCGTG |
| | | CGTGGAGGCCGACGACGCCGTGTGCCGCTGCGCCTACGGCTACTACCAGG |
| | | ATGAGACGACTGGGCGCTGCGAGGCGTGCCGCGTGTGCGAGGCGGGCTC |
| | | GGGCCTCGTGTTCTCCTGCCAGGACAAGCAGAACACCGTGTGCGAGGAGT |
| | | GCCCCGACGGCACGTATTCCGACGAGGCCAACCACGTGGACCCGTGCCTG |
| | | CCCTGCACCGTGTGCGAGGACACCGAGCGCCAGCTCCGCGAGTGCACAC |
| | | GCTGGGCCGACGCCGAGTGCGAGGAGATCCCTGGCCGTTGGATTACACG |
| | | GTCCACACCCCCAGAGGGCTCGGACAGCACAGCCCCCAGCACCCAGGAG |
| | | CCTGAGGCACCTCCAGAACAAGACCTCATAGCCAGCACGGTGGCAGGTG |
| | | TGGTGACCACAGTGATGGGCAGCTCCCAGCCCGTGGTGACCCGAGGCAC |
| | | CACCGACAACCTCATCCCTGTCTATTGCTCCATCCTGGCTGCTGTGGTTGT |
| | | GGGTCTTGTGGCCTACATAGCCTTCAAGAGG |
| 119 | μDISC: μCISC- | ATGCCTCTGGGCCTGCTGTGGCTGGGCCTGGCCCTGCTGGGCGCCCTGCA |
| | FRB; nucleotide | CGCCCAGGCCGGCGTGCAGGTGGAGACAATCTCCCCAGGCGACGGACGC |
| | sequence | ACATTCCCTAAGCGGGGCCAGACCTGCGTGGTGCACTATACAGGCATGCT |
| | | GGAGGATGGCAAGAAGTTTGACAGCTCCCGGGATAGAAACAAGCCATTC |
| | | AAGTTTATGCTGGGCAAGCAGGAAGTGATCAGAGGCTGGGAGGAGGGCG |
| | | TGGCCCAGATGTCTGTGGGCCAGAGGGCCAAGCTGACCATCAGCCCAGA |
| | | CTACGCCTATGGAGCAACAGGCCACCCAGGAATCATCCCACCTCACGCCA |
| | | CCCTGGTGTTCGATGTGGAGCTGCTGAAGCTGGGCGAGGGAGGGTCACCT |
| | | GGATCCAACACATCAAAAGAGAACCCCTTTCTGTTCGCATTGGAGGCCGT |
| | | AGTCATATCTGTTGGATCCATGGGACTTATTATCTCCCTGTTGTGTGTGTA |
| | | CTTCTGGCTGGAACGGACTATGCCCAGGATCCCCACGCTCAAGAATCTGG |
| | | AAGATCTCGTCACAGAATACCATGGTAATTTCAGCGCCTGGAGCGGAGTC |
| | | TCTAAGGGTCTGGCCGAATCCCTCCAACCCGATTATTCTGAACGGTTGTG |
| | | CCTCGTATCCGAAATACCACCAAAAGGCGGGGCTCTGGGTGAGGGCCCA |
| | | GGGGCGAGTCCGTGCAATCAACACAGCCCGTATTGGGCCCCTCCTTGTTA |
| | | TACGTTGAAGCCCGAAACTGGAAGCGGAGCTACTAACTTCAGCCTGCTGA |
| | | AGCAGGCTGGAGACGTGGAGGAGAACCCTGGACCTATGGCACTGCCCGT |
| | | GACCGCCCTGCTGCTGCCTCTGGCCCTGCTGCTGCACGCAGCCCGGCCTA |
| | | TCCTGTGGCACGAGATGTGGCACGAGGGCCTGGAGGAGGCCAGCAGGCT |
| | | GTATTTTGGCGAGCGCAACGTGAAGGGCATGTTCGAGGTGCTGGAGCCTC |
| | | TGCACGCCATGATGGAGAGAGGCCCACAGACCCTGAAGGAGACATCCTT |
| | | TAACCAGGCCTATGGACGGGACCTGATGGAGGCACAGGAGTGGTGCAGA |
| | | AAGTACATGAAGTCTGGCAATGTGAAGGACCTGCTGCAGGCCTGGGATCT |
| | | GTACTATCACGTGTTTCGGAGAATCTCCAAGCCAGCAGCTCTCGGCAAAG |
| | | ACACGATTCCGTGGCTTGGGCATCTGCTCGTTGGGCTGAGCGGTGCGTTT |
| | | GGTTTCATCATCTTGGTCTATCTCTTGATCAATTGCAGAAATACAGGCCCT |
| | | TGGCTGAAAAAAGTGCTCAAGTGTAATACCCCCGACCCAAGCAAGTTCTT |
| | | CTCCCAGCTTTCTTCAGAGCATGGAGGCGATGTGCAGAAATGGCTCTCTT |
| | | CACCTTTTCCCTCCTCAAGCTTCTCCCCGGGAGGGCTGGCGCCCGAGATTT |
| | | CACCTCTTGAGGTACTTGAACGAGACAAGGTTACCCAACTTCTCCTTCAA |
| | | CAGGATAAGGTACCCGAACCTGCGAGCCTTAGCTTGAATACAGACGCTTA |
| | | TCTCTCACTGCAGGAACTGCAAGGATCTGGTGCTACTAATTTTTCTCTTTT |
| | | GAAGCAAGCTGGAGATGTTGAAGAGAACCCCGGTCCGGAGATGTGGCAT |
| | | GAGGGTCTGGAAGAAGCGTCTCGACTGTACTTTGGTGAGCGCAATGTGAA |
| | | GGGCATGTTTGAAGTCCTCGAACCCCTTCATGCCATGATGGAACGCGGAC |
| | | CCCAGACCTTGAAGGAGACAAGTTTTAACCAAGCTTACGGAAGAGACCT |
| | | GATGGAAGCCCAGGAATGGTGCAGGAAATACATGAAAAGCGGGAATGTG |
| | | AAGGACTTGCTCCAAGCGTGGGACCTGTACTATCATGTCTTTAGGCGCAT |
| | | TAGTAAG |
| 120 | μDISC: μCISC- | MPLGLLWLGLALLGALHAQAGVQVETISPGDGRTFPKRGQTCVVHYTGMLE |
| | FRB amino acid | DGKKFDSSRDRNKPFKFMLGKQEVIRGWEEGVAQMSVGQRAKLTISPDYAY |
| | sequence | GATGHPGIIPPHATLVFDVELLKLGEGGSPGSNTSKENPFLFALEAVVISVGS |
| | | MGLIISLLCVYFWLERTMPRIPTLKNLEDLVTEYHGNFSAWSGVSKGLAESL |
| | | QPDYSERLCLVSEIPPKGGALGEGPGASPCNQHSPYWAPPCYTLKPETGSGAT |
| | | NFSLLKQAGDVEENPGPMALPVTALLLPLALLLHAARPILWHEMWHEGLEE |
| | | ASRLYFGERNVKGMFEVLEPLHAMMERGPQTLKETSFNQAYGRDLMEAQE |
| | | WCRKYMKSGNVKDLLQAWDLYYHVFRRISKPAALGKDTIPWLGHLLVGLS |
| | | GAFGFIILVYLLINCRNTGPWLKKVLKCNTPDPSKFFSQLSSEHGGDVQKWLS |
| | | SPFPSSSFSPGGLAPEISPLEVLERDKVTQLLLQQDKVPEPASLSLNTDAYLSL |
| | | QELQGSGATNFSLLKQAGDVEENPGPEMWHEGLEEASRLYFGERNVKGMFE |
| | | VLEPLHAMMERGPQTLKETSFNQAYGRDLMEAQEWCRKYMKSGNVKDLL |
| | | QAWDLYYHVFRRISK |
| 121 | FRB; nucleotide | GAGATGTGGCATGAGGGTCTGGAAGAAGCGTCTCGACTGTACTTTGGTGA |
| | sequence | GCGCAATGTGAAGGGCATGTTTGAAGTCCTCGAACCCCTTCATGCCATGA |
| | | TGGAACGCGGACCCCAGACCTTGAAGGAGACAAGTTTTAACCAAGCTTA |
| | | CGGAAGAGACCTGATGGAAGCCCAGGAATGGTGCAGGAAATACATGAAA |
| | | AGCGGGAATGTGAAGGACTTGCTCCAAGCGTGGGACCTGTACTATCATGT |
| | | CTTTAGGCGCATTAGTAAG |
| 122 | FRB amino acid | EMWHEGLEEASRLYFGERNVKGMFEVLEPLHAMMERGPQTLKETSFNQAY |
| | sequence | GRDLMEAQEWCRKYMKSGNVKDLLQAWDLYYHVFRRISK |
| 123 | CISCβ: FRB- | ATGGCACTGCCCGTGACCGCCCTGCTGCTGCCTCTGGCCCTGCTGCTGCA |
| | IL2Rβ | CGCAGCCCGGCCTATCCTGTGGCACGAGATGTGGCACGAGGGCCTGGAG |
| | nucleotide | GAGGCCAGCAGGCTGTATTTTGGCGAGCGCAACGTGAAGGGCATGTTCG |
| | sequence | AGGTGCTGGAGCCTCTGCACGCCATGATGGAGAGAGGCCCACAGACCCT |
| | | GAAGGAGACATCCTTTAACCAGGCCTATGGACGGGACCTGATGGAGGCA |
| | | CAGGAGTGGTGCAGAAAGTACATGAAGTCTGGCAATGTGAAGGACCTGC |
| | | TGCAGGCCTGGGATCTGTACTATCACGTGTTTCGGAGAATCTCCAAGCCA |
| | | GCAGCTCTCGGCAAAGACACGATTCCGTGGCTTGGGCATCTGCTCGTTGG |
| | | GCTGAGCGGTGCGTTTGGTTTCATCATCTTGGTCTATCTCTTGATCAATTG |
| | | CAGAAATACAGGCCCTTGGCTGAAAAAAGTGCTCAAGTGTAATACCCCC |
| | | GACCCAAGCAAGTTCTTCTCCCAGCTTTCTTCAGAGCATGGAGGCGATGT |
| | | GCAGAAATGGCTCTCTTCACCTTTTCCCTCCTCAAGCTTCTCCCCGGGAGG |
| | | GCTGGCGCCCGAGATTTCACCTCTTGAGGTACTTGAACGAGACAAGGTTA |
| | | CCCAACTTCTCCTTCAACAGGATAAGGTACCCGAACCTGCGAGCCTTAGC |
| | | TCCAACCACTCTCTTACGAGCTGCTTCACCAATCAGGGATACTTCTTTTTC |
| | | CACCTTCCCGATGCGCTGGAAATCGAAGCTTGTCAAGTTTACTTTACCTAT |
| | | GATCCATATAGCGAGGAAGATCCCGACGAAGGAGTCGCCGGTGCGCCCA |
| | | CGGGTTCCTCACCCCAACCTCTCCAGCCTCTCTCAGGAGAAGATGATGCT |
| | | TATTGCACTTTTCCCAGTAGAGACGATCTCCTCCTCTTTTCTCCATCTCTTT |
| | | TGGGGGGACCTTCCCCCCCTTCTACGGCACCTGGCGGGTCTGGTGCTGGC |
| | | GAGGAGCGGATGCCGCCGTCCCTCCAGGAGCGAGTACCACGAGATTGGG |
| | | ATCCCCAGCCACTTGGACCCCCCACCCCCGGCGTACCTGACCTTGTCGAT |
| | | TTTCAACCTCCCCCTGAATTGGTGCTGCGAGAGGCTGGGGAGGAAGTTCC |
| | | GGACGCTGGGCCGAGGGAGGGCGTGTCCTTTCCATGGAGTAGGCCTCCA |
| | | GGTCAAGGCGAGTTTAGGGCTCTCAACGCGCGGCTGCCGTTGAATACAGA |
| | | CGCTTATCTCTCACTGCAGGAACTGCAAGGTCAGGACCCAACACATCTTG |
| | | TA |
| 124 | CISCβ: FRB- | MALPVTALLLPLALLLHAARPILWHEMWHEGLEEASRLYFGERNVKGMFEV |
| | IL2Rβ amino | LEPLHAMMERGPQTLKETSFNQAYGRDLMEAQEWCRKYMKSGNVKDLLQ |
| | acid sequence | AWDLYYHVFRRISKPAALGKDTIPWLGHLLVGLSGAFGFIILVYLLINCRNTG |
| | | PWLKKVLKCNTPDPSKFFSQLSSEHGGDVQKWLSSPFPSSSFSPGGLAPEISPL |
| | | EVLERDKVTQLLLQQDKVPEPASLSSNHSLTSCFTNQGYFFFHLPDALEIEAC |
| | | QVYFTYDPYSEEDPDEGVAGAPTGSSPQPLQPLSGEDDAYCTFPSRDDLLLFS |
| | | PSLLGGPSPPSTAPGGSGAGEERMPPSLQERVPRDWDPQPLGPPTPGVPDLVD |
| | | FQPPPELVLREAGEEVPDAGPREGVSFPWSRPPGQGEFRALNARLPLNTDAY |
| | | LSLQELQGQDPTHLV |
| 125 | TCRa guide 1 | ATGCAAGCCCATAACCGCTG |
| 126 | TCRa guide 2 | CAAGAGGCCACAGCGGTTAT |
| 127 | TCRa guide 3 | CCAAGAGGCCACAGCGGTTA |
| 128 | TCRa guide 4 | TTCGGAACCCAATCACTGAC |
| 129 | primer mix for | GGCACCTCCAGAACAAGACC |
| | insert forward | |
| 130 | primer mix for | TCCTGATCCTCACTGTTCTGTGTC |
| | insert reverse | |
| 131 | primer mix for | AGACCCACAACCACAGCAGC |
| | insert probe- | |
| | FAM | |
| 132 | primer mix for | GTTCACACGCATGTTTGCCT |
| | control forward | |
| 133 | primer mix for | ATCCTGAGGGTACTGACGCT |
| | control reverse | |
| 134 | primer mix for | TGGCGGTGACTGGGATGGC |
| | control probe- | |
| | Hex | |
| 135 | #3017 | GTAGAAAAGATCAAAGGATCTTCTTGAGATCCTTTTTTTCTGCGCGTAATC |
| | pAAV_FOXP3. | TGCTGCTTGCAAACAAAAAAACCACCGCTACCAGCGGTGGTTTGTTTGCC |
| | 025_MND.FOX | GGATCAAGAGCTACCAACTCTTTTTCCGAAGGTAACTGGCTTCAGCAGAG |
| | P3geneartCDS.p | CGCAGATACCAAATACTGTCCTTCTAGTGTAGCCGTAGTTAGGCCACCAC |
| | 2A.GFP.WPRE3 | TTCAAGAACTCTGTAGCACCGCCTACATACCTCGCTCTGCTAATCCTGTTA |
| | .pA_025 | CCAGTGGCTGCTGCCAGTGGCGATAAGTCGTGTCTTACCGGGTTGGACTC |
| | | AAGACGATAGTTACCGGATAAGGCGCAGCGGTCGGGCTGAACGGGGGGT |
| | | TCGTGCACACAGCCCAGCTTGGAGCGAACGACCTACACCGAACTGAGAT |
| | | ACCTACAGCGTGAGCTATGAGAAAGCGCCACGCTTCCCGAAGGGAGAAA |
| | | GGCGGACAGGTATCCGGTAAGCGGCAGGGTCGGAACAGGAGAGCGCACG |
| | | AGGGAGCTTCCAGGGGGAAACGCCTGGTATCTTTATAGTCCTGTCGGGTT |
| | | TCGCCACCTCTGACTTGAGCGTCGATTTTTGTGATGCTCGTCAGGGGGGC |
| | | GGAGCCTATGGAAAAACGCCAGCAACGCGGCCTTTTTACGGTTCCTGGCC |
| | | TTTTGCTGGCCTTTTGCTCACATGTTCTTTCCTGCGTTATCCCCTGATTCTG |
| | | TGGATAACCGTATTACCGCCTTTGAGTGAGCTGATACCGCTCGCCGCAGC |
| | | CGAACGACCGAGCGCAGCGAGTCAGTGAGCGAGGAAGCGGAAGAGCGC |
| | | CCAATACGCAAACCGCCTCTCCCCGCGCGTTGGCCGATTCATTAATGCAG |
| | | CTGCGCGCTCGCTCGCTCACTGAGGCCGCCCGGGCAAAGCCCGGGCGTCG |
| | | GGCGACCTTTGGTCGCCCGGCCTCAGTGAGCGAGCGAGCGCGCAGAGAG |
| | | GGAGTGGCCAACTCCATCACTAGGGGTTCCTTGTAGTTAATGATTAACCC |
| | | GCCATGCTACTTATCTACGTAGCGGCCGCTGCTAGCGTGGGCAGGCAAGC |
| | | CAGGTGCTGGACCTCTGCACGTGGGGCATGTGTGGGTATGTACATGTACC |
| | | TGTGTTCTTGGTGTGTGTGTGTGTGTGTGTGTGTGTGTGTGTCTAGAGCTG |
| | | GGGTGCAACTATGGGGCCCCTCGGGACATGTCCCAGCCAATGCCTGCTTT |
| | | GACCAGAGGAGTGTCCACGTGGCTCAGGTGGTCGAGTATCTCATACCGCC |
| | | CTAGCACACGTGTGACTCCTTTCCCCTATTGTCTACACGCGTAGGAACAG |
| | | AGAAACAGGAGAATATGGGCCAAACAGGATATCTGTGGTAAGCAGTTCC |
| | | TGCCCCGGCTCAGGGCCAAGAACAGTTGGAACAGCAGAATATGGGCCAA |
| | | ACAGGATATCTGTGGTAAGCAGTTCCTGCCCCGGCTCAGGGCCAAGAACA |
| | | GATGGTCCCCAGATGCGGTCCCGCCCTCAGCAGTTTCTAGAGAACCATCA |
| | | GATGTTTCCAGGGTGCCCCAAGGACCTGAAATGACCCTGTGCCTTATTTG |
| | | AACTAACCAATCAGTTCGCTTCTCGCTTCTGTTCGCGCGCTTCTGCTCCCC |
| | | GAGCTCTATATAAGCAGAGCTCGTTTAGTGAACCGTCAGATCGCCTGGAG |
| | | ACGCCATCCACGCTGTTTTGACTTCCATAGAAGGATCTCGAGGCCACCAT |
| | | GCCTAATCCTCGGCCTGGAAAGCCTAGCGCTCCTTCTCTTGCTCTGGGACC |
| | | TTCTCCTGGCGCCTCTCCATCTTGGAGAGCCGCTCCTAAAGCCAGCGATCT |
| | | GCTGGGAGCTAGAGGACCTGGCGGCACATTTCAGGGCAGAGATCTTAGA |
| | | GGCGGAGCCCACGCTAGCTCCTCCAGCCTTAATCCTATGCCTCCTAGCCA |
| | | GCTCCAGCTGCCTACACTGCCTCTGGTTATGGTGGCTCCTAGCGGAGCTA |
| | | GACTGGGCCCTCTGCCTCATCTGCAAGCTCTGCTGCAGGACAGACCCCAC |
| | | TTCATGCACCAGCTGAGCACCGTGGATGCCCACGCAAGAACACCTGTGCT |
| | | GCAGGTTCACCCTCTGGAATCCCCAGCCATGATCAGCCTGACACCTCCAA |
| | | CAACAGCCACCGGCGTGTTCAGCCTGAAAGCCAGACCTGGACTGCCTCCT |
| | | GGCATCAATGTGGCCAGCCTGGAATGGGTGTCCAGAGAACCTGCTCTGCT |
| | | GTGCACATTCCCCAATCCAAGCGCTCCCAGAAAGGACAGCACACTGTCTG |
| | | CCGTGCCTCAGAGCAGCTATCCCCTGCTTGCTAACGGCGTGTGCAAGTGG |
| | | CCTGGATGCGAGAAGGTGTTCGAGGAACCCGAGGACTTCCTGAAGCACT |
| | | GCCAGGCCGATCATCTGCTGGACGAGAAAGGCAGAGCCCAGTGTCTGCT |
| | | CCAGCGCGAGATGGTGCAGTCTCTGGAACAGCAGCTGGTCCTGGAAAAA |
| | | GAAAAGCTGAGCGCCATGCAGGCCCACCTGGCCGGAAAAATGGCCCTGA |
| | | CAAAGGCCAGCAGCGTGGCCTCTTCTGATAAGGGCAGCTGCTGCATTGTG |
| | | GCCGCTGGATCTCAGGGACCTGTGGTTCCTGCTTGGAGCGGACCTAGAGA |
| | | GGCCCCTGATTCTCTGTTTGCCGTGCGGAGACACCTGTGGGGCTCTCACG |
| | | GCAACTCTACTTTCCCCGAGTTCCTGCACAACATGGACTACTTCAAGTTCC |
| | | ACAACATGCGGCCTCCATTCACCTACGCCACACTGATCAGATGGGCCATT |
| | | CTGGAAGCCCCTGAGAAGCAGAGAACCCTGAACGAGATCTACCACTGGT |
| | | TTACCCGGATGTTCGCCTTCTTCCGGAATCACCCTGCCACCTGGAAGAAC |
| | | GCCATCCGGCACAATCTGAGCCTGCACAAGTGCTTCGTGCGCGTGGAATC |
| | | TGAGAAAGGCGCCGTGTGGACAGTGGACGAGCTGGAATTCAGAAAGAAG |
| | | AGAAGCCAGCGGCCTAGCCGGTGCAGCAATCCTACACCTGGACCTGGAA |
| | | GCGGAGCGACTAACTTCAGCCTGCTGAAGCAGGCCGGAGATGTGGAGGA |
| | | AAACCCTGGACCGATGGTGAGCAAGGGCGAGGAGCTGTTCACCGGGGTG |
| | | GTGCCCATCCTGGTCGAGCTGGACGGCGACGTAAACGGCCACAAGTTCA |
| | | GCGTGTCTGGCGAGGGCGAGGGCGATGCCACCTACGGCAAGCTGACCCT |
| | | GAAGTTCATCTGCACCACCGGCAAGCTGCCCGTGCCCTGGCCCACCCTCG |
| | | TGACCACCCTGACCTACGGCGTGCAGTGCTTCAGCCGCTACCCCGACCAC |
| | | ATGAAGCAGCACGACTTCTTCAAGTCCGCCATGCCCGAAGGCTACGTCCA |
| | | GGAGCGCACCATCTTCTTCAAGGACGACGGCAACTACAAGACCCGCGCC |
| | | GAGGTGAAGTTCGAGGGCGACACCCTGGTGAACCGCATCGAGCTGAAGG |
| | | GCATCGACTTCAAGGAGGACGGCAACATCCTGGGGCACAAGCTGGAGTA |
| | | CAACTACAACAGCCACAACGTCTATATCATGGCCGACAAGCAGAAGAAC |
| | | GGCATCAAGGCGAACTTCAAGATCCGCCACAACATCGAGGACGGCAGCG |
| | | TGCAGCTCGCCGACCACTACCAGCAGAACACCCCCATCGGCGACGGCCCC |
| | | GTGCTGCTGCCCGACAACCACTACCTGAGCACCCAGTCCGCCCTGAGCAA |
| | | AGACCCCAACGAGAAGCGCGATCACATGGTCCTGCTGGAGTTCGTGACC |
| | | GCCGCCGGGATCACTCTCGGCATGGACGAGCTGTACAAGTAATGAAAGC |
| | | TTCCACGGAATTGTCAGTGCCCAACAGCCGAGCCCCTGTCCAGCAGCGGG |
| | | CAAGGCAGGCGGCGATGAGTTCCGCCGTGGCAAGAACTAACCAGGATTT |
| | | ATACAAGGAGGAGAAAATGAAAGCCATACGGGAAGCAATAGCATGATAC |
| | | AAAGGCATTAAAGCAGCGTATCCACATAGCGTAAAAGGAGCAACATAGT |
| | | TAAGAATACCAGTCAATCTTTCACAAATTTTGTAATCCAGAGGTTGATTA |
| | | TCGTCGACTGCTTTATTTGTGAAATTTGTGATGCTATTGCTTTATTTGTAA |
| | | CCATTATAAGCTGCAATAAACAAGTTAACAACAACAATTGCATTCATTTT |
| | | ATGTTTCAGGTTCAGGGGGAGATGTGGGAGGTTTTTTAAAGCACTAGTGT |
| | | GAGGCCCTGGGCCCAGGATGGGGCAGGCAGGGTGGGGTACCTGGACCTA |
| | | CAGGTGCCGACCTTTACTGTGGCACTGGGCGGGAGGGGGGCTGGCTGGG |
| | | GCACAGGAAGTGGTTTCTGGGTCCCAGGCAAGTCTGTGACTTATGCAGAT |
| | | GTTGCAGGGCCAAGAAAATCCCCACCTGCCAGGCCTCAGAGATTGGAGG |
| | | CTCTCCCCGACCTCCCAATCCCTGTCTCAGGAGAGGAGGAGGCCGTGGAT |
| | | CCTACGTAGATAAGTAGCATGGCGGGTTAATCATTAACTACAAGGAACCC |
| | | CTAGTGATGGAGTTGGCCACTCCCTCTCTGCGCGCTCGCTCGCTCACTGA |
| | | GGCCGGGCGACCAAAGGTCGCCCGACGCCCGGGCTTTGCCCGGGCGGCC |
| | | TCAGTGAGCGAGCGAGCGCGCCAGCTGGCGTAATAGCGAAGAGGCCCGC |
| | | ACCGATCGCCCTTCCCAACAGTTGCGCAGCCTGAATGGCGAATGGCGATT |
| | | CCGTTGCAATGGCTGGCGGTAATATTGTTCTGGATATTACCAGCAAGGCC |
| | | GATAGTTTGAGTTCTTCTACTCAGGCAAGTGATGTTATTACTAATCAAAG |
| | | AAGTATTGCGACAACGGTTAATTTGCGTGATGGACAGACTCTTTTACTCG |
| | | GTGGCCTCACTGATTATAAAAACACTTCTCAGGATTCTGGCGTACCGTTC |
| | | CTGTCTAAAATCCCTTTAATCGGCCTCCTGTTTAGCTCCCGCTCTGATTCT |
| | | AACGAGGAAAGCACGTTATACGTGCTCGTCAAAGCAACCATAGTACGCG |
| | | CCCTGTAGCGGCGCATTAAGCGCGGCGGGTGTGGTGGTTACGCGCAGCGT |
| | | GACCGCTACACTTGCCAGCGCCCTAGCGCCCGCTCCTTTCGCTTTCTTCCC |
| | | TTCCTTTCTCGCCACGTTCGCCGGCTTTCCCCGTCAAGCTCTAAATCGGGG |
| | | GCTCCCTTTAGGGTTCCGATTTAGTGCTTTACGGCACCTCGACCCCAAAA |
| | | AACTTGATTAGGGTGATGGTTCACGTAGTGGGCCATCGCCCTGATAGACG |
| | | GTTTTTCGCCCTTTGACGTTGGAGTCCACGTTCTTTAATAGTGGACTCTTG |
| | | TTCCAAACTGGAACAACACTCAACCCTATCTCGGTCTATTCTTTTGATTTA |
| | | TAAGGGATTTTGCCGATTTCGGCCTATTGGTTAAAAAATGAGCTGATTTA |
| | | ACAAAAATTTAACGCGAATTTTAACAAAATATTAACGTTTACAATTTAAA |
| | | TATTTGCTTATACAATCTTCCTGTTTTTGGGGCTTTTCTGATTATCAACCGG |
| | | GGTACATATGATTGACATGCTAGTTTTACGATTACCGTTCATCGATTCTCT |
| | | TGTTTGCTCCAGACTCTCAGGCAATGACCTGATAGCCTTTGTAGAGACCT |
| | | CTCAAAAATAGCTACCCTCTCCGGCATGAATTTATCAGCTAGAACGGTTG |
| | | AATATCATATTGATGGTGATTTGACTGTCTCCGGCCTTTCTCACCCGTTTG |
| | | AATCTTTACCTACACATTACTCAGGCATTGCATTTAAAATATATGAGGGTT |
| | | CTAAAAATTTTTATCCTTGCGTTGAAATAAAGGCTTCTCCCGCAAAAGTA |
| | | TTACAGGGTCATAATGTTTTTGGTACAACCGATTTAGCTTTATGCTCTGAG |
| | | GCTTTATTGCTTAATTTTGCTAATTCTTTGCCTTGCCTGTATGATTTATTGG |
| | | ATGTTGGAATCGCCTGATGCGGTATTTTCTCCTTACGCATCTGTGCGGTAT |
| | | TTCACACCGCATATGGTGCACTCTCAGTACAATCTGCTCTGATGCCGCAT |
| | | AGTTAAGCCAGCCCCGACACCCGCCAACACCCGCTGACGCGCCCTGACG |
| | | GGCTTGTCTGCTCCCGGCATCCGCTTACAGACAAGCTGTGACCGTCTCCG |
| | | GGAGCTGCATGTGTCAGAGGTTTTCACCGTCATCACCGAAACGCGCGAGA |
| | | CGAAAGGGCCTCGTGATACGCCTATTTTTATAGGTTAATGTCATGATAAT |
| | | AATGGTTTCTTAGACGTCAGGTGGCACTTTTCGGGGAAATGTGCGCGGAA |
| | | CCCCTATTTGTTTATTTTTCTAAATACATTCAAATATGTATCCGCTCATGA |
| | | GACAATAACCCTGATAAATGCTTCAATAATATTGAAAAAGGAAGAGTAT |
| | | GAGTATTCAACATTTCCGTGTCGCCCTTATTCCCTTTTTTGCGGCATTTTGC |
| | | CTTCCTGTTTTTGCTCACCCAGAAACGCTGGTGAAAGTAAAAGATGCTGA |
| | | AGATCAGTTGGGTGCACGAGTGGGTTACATCGAACTGGATCTCAACAGCG |
| | | GTAAGATCCTTGAGAGTTTTCGCCCCGAAGAACGTTTTCCAATGATGAGC |
| | | ACTTTTAAAGTTCTGCTATGTGGCGCGGTATTATCCCGTATTGACGCCGGG |
| | | CAAGAGCAACTCGGTCGCCGCATACACTATTCTCAGAATGACTTGGTTGA |
| | | GTACTCACCAGTCACAGAAAAGCATCTTACGGATGGCATGACAGTAAGA |
| | | GAATTATGCAGTGCTGCCATAACCATGAGTGATAACACTGCGGCCAACTT |
| | | ACTTCTGACAACGATCGGAGGACCGAAGGAGCTAACCGCTTTTTTGCACA |
| | | ACATGGGGGATCATGTAACTCGCCTTGATCGTTGGGAACCGGAGCTGAAT |
| | | GAAGCCATACCAAACGACGAGCGTGACACCACGATGCCTGTAGCAATGG |
| | | CAACAACGTTGCGCAAACTATTAACTGGCGAACTACTTACTCTAGCTTCC |
| | | CGGCAACAATTAATAGACTGGATGGAGGCGGATAAAGTTGCAGGACCAC |
| | | TTCTGCGCTCGGCCCTTCCGGCTGGCTGGTTTATTGCTGATAAATCTGGAG |
| | | CCGGTGAGCGTGGGTCTCGCGGTATCATTGCAGCACTGGGGCCAGATGGT |
| | | AAGCCCTCCCGTATCGTAGTTATCTACACGACGGGGAGTCAGGCAACTAT |
| | | GGATGAACGAAATAGACAGATCGCTGAGATAGGTGCCTCACTGATTAAG |
| | | CATTGGTAACTGTCAGACCAAGTTTACTCATATATACTTTAGATTGATTTA |
| | | AAACTTCATTTTTAATTTAAAAGGATCTAGGTGAAGATCCTTTTTGATAAT |
| | | CTCATGACCAAAATCCCTTAACGTGAGTTTTCGTTCCACTGAGCGTCAGA |
| | | CCCC |
| 136 | #3018_pAAV_F | GTAGAAAAGATCAAAGGATCTTCTTGAGATCCTTTTTTTCTGCGCGTAATC |
| | OXP3.025_MN | TGCTGCTTGCAAACAAAAAAACCACCGCTACCAGCGGTGGTTTGTTTGCC |
| | D.FOXP3genear | GGATCAAGAGCTACCAACTCTTTTTCCGAAGGTAACTGGCTTCAGCAGAG |
| | tCDS.P2A.GFP. | CGCAGATACCAAATACTGTCCTTCTAGTGTAGCCGTAGTTAGGCCACCAC |
| | WPRE6.pA_025 | TTCAAGAACTCTGTAGCACCGCCTACATACCTCGCTCTGCTAATCCTGTTA |
| | | CCAGTGGCTGCTGCCAGTGGCGATAAGTCGTGTCTTACCGGGTTGGACTC |
| | | AAGACGATAGTTACCGGATAAGGCGCAGCGGTCGGGCTGAACGGGGGGT |
| | | TCGTGCACACAGCCCAGCTTGGAGCGAACGACCTACACCGAACTGAGAT |
| | | ACCTACAGCGTGAGCTATGAGAAAGCGCCACGCTTCCCGAAGGGAGAAA |
| | | GGCGGACAGGTATCCGGTAAGCGGCAGGGTCGGAACAGGAGAGCGCACG |
| | | AGGGAGCTTCCAGGGGGAAACGCCTGGTATCTTTATAGTCCTGTCGGGTT |
| | | TCGCCACCTCTGACTTGAGCGTCGATTTTTGTGATGCTCGTCAGGGGGGC |
| | | GGAGCCTATGGAAAAACGCCAGCAACGCGGCCTTTTTACGGTTCCTGGCC |
| | | TTTTGCTGGCCTTTTGCTCACATGTTCTTTCCTGCGTTATCCCCTGATTCTG |
| | | TGGATAACCGTATTACCGCCTTTGAGTGAGCTGATACCGCTCGCCGCAGC |
| | | CGAACGACCGAGCGCAGCGAGTCAGTGAGCGAGGAAGCGGAAGAGCGC |
| | | CCAATACGCAAACCGCCTCTCCCCGCGCGTTGGCCGATTCATTAATGCAG |
| | | CTGCGCGCTCGCTCGCTCACTGAGGCCGCCCGGGCAAAGCCCGGGCGTCG |
| | | GGCGACCTTTGGTCGCCCGGCCTCAGTGAGCGAGCGAGCGCGCAGAGAG |
| | | GGAGTGGCCAACTCCATCACTAGGGGTTCCTTGTAGTTAATGATTAACCC |
| | | GCCATGCTACTTATCTACGTAGCGGCCGCTGCTAGCGTGGGCAGGCAAGC |
| | | CAGGTGCTGGACCTCTGCACGTGGGGCATGTGTGGGTATGTACATGTACC |
| | | TGTGTTCTTGGTGTGTGTGTGTGTGTGTGTGTGTGTGTGTGTCTAGAGCTG |
| | | GGGTGCAACTATGGGGCCCCTCGGGACATGTCCCAGCCAATGCCTGCTTT |
| | | GACCAGAGGAGTGTCCACGTGGCTCAGGTGGTCGAGTATCTCATACCGCC |
| | | CTAGCACACGTGTGACTCCTTTCCCCTATTGTCTACACGCGTAGGAACAG |
| | | AGAAACAGGAGAATATGGGCCAAACAGGATATCTGTGGTAAGCAGTTCC |
| | | TGCCCCGGCTCAGGGCCAAGAACAGTTGGAACAGCAGAATATGGGCCAA |
| | | ACAGGATATCTGTGGTAAGCAGTTCCTGCCCCGGCTCAGGGCCAAGAACA |
| | | GATGGTCCCCAGATGCGGTCCCGCCCTCAGCAGTTTCTAGAGAACCATCA |
| | | GATGTTTCCAGGGTGCCCCAAGGACCTGAAATGACCCTGTGCCTTATTTG |
| | | AACTAACCAATCAGTTCGCTTCTCGCTTCTGTTCGCGCGCTTCTGCTCCCC |
| | | GAGCTCTATATAAGCAGAGCTCGTTTAGTGAACCGTCAGATCGCCTGGAG |
| | | ACGCCATCCACGCTGTTTTGACTTCCATAGAAGGATCTCGAGGCCACCAT |
| | | GCCTAATCCTCGGCCTGGAAAGCCTAGCGCTCCTTCTCTTGCTCTGGGACC |
| | | TTCTCCTGGCGCCTCTCCATCTTGGAGAGCCGCTCCTAAAGCCAGCGATCT |
| | | GCTGGGAGCTAGAGGACCTGGCGGCACATTTCAGGGCAGAGATCTTAGA |
| | | GGCGGAGCCCACGCTAGCTCCTCCAGCCTTAATCCTATGCCTCCTAGCCA |
| | | GCTCCAGCTGCCTACACTGCCTCTGGTTATGGTGGCTCCTAGCGGAGCTA |
| | | GACTGGGCCCTCTGCCTCATCTGCAAGCTCTGCTGCAGGACAGACCCCAC |
| | | TTCATGCACCAGCTGAGCACCGTGGATGCCCACGCAAGAACACCTGTGCT |
| | | GCAGGTTCACCCTCTGGAATCCCCAGCCATGATCAGCCTGACACCTCCAA |
| | | CAACAGCCACCGGCGTGTTCAGCCTGAAAGCCAGACCTGGACTGCCTCCT |
| | | GGCATCAATGTGGCCAGCCTGGAATGGGTGTCCAGAGAACCTGCTCTGCT |
| | | GTGCACATTCCCCAATCCAAGCGCTCCCAGAAAGGACAGCACACTGTCTG |
| | | CCGTGCCTCAGAGCAGCTATCCCCTGCTTGCTAACGGCGTGTGCAAGTGG |
| | | CCTGGATGCGAGAAGGTGTTCGAGGAACCCGAGGACTTCCTGAAGCACT |
| | | GCCAGGCCGATCATCTGCTGGACGAGAAAGGCAGAGCCCAGTGTCTGCT |
| | | CCAGCGCGAGATGGTGCAGTCTCTGGAACAGCAGCTGGTCCTGGAAAAA |
| | | GAAAAGCTGAGCGCCATGCAGGCCCACCTGGCCGGAAAAATGGCCCTGA |
| | | CAAAGGCCAGCAGCGTGGCCTCTTCTGATAAGGGCAGCTGCTGCATTGTG |
| | | GCCGCTGGATCTCAGGGACCTGTGGTTCCTGCTTGGAGCGGACCTAGAGA |
| | | GGCCCCTGATTCTCTGTTTGCCGTGCGGAGACACCTGTGGGGCTCTCACG |
| | | GCAACTCTACTTTCCCCGAGTTCCTGCACAACATGGACTACTTCAAGTTCC |
| | | ACAACATGCGGCCTCCATTCACCTACGCCACACTGATCAGATGGGCCATT |
| | | CTGGAAGCCCCTGAGAAGCAGAGAACCCTGAACGAGATCTACCACTGGT |
| | | TTACCCGGATGTTCGCCTTCTTCCGGAATCACCCTGCCACCTGGAAGAAC |
| | | GCCATCCGGCACAATCTGAGCCTGCACAAGTGCTTCGTGCGCGTGGAATC |
| | | TGAGAAAGGCGCCGTGTGGACAGTGGACGAGCTGGAATTCAGAAAGAAG |
| | | AGAAGCCAGCGGCCTAGCCGGTGCAGCAATCCTACACCTGGACCTGGAA |
| | | GCGGAGCGACTAACTTCAGCCTGCTGAAGCAGGCCGGAGATGTGGAGGA |
| | | AAACCCTGGACCGATGGTGAGCAAGGGCGAGGAGCTGTTCACCGGGGTG |
| | | GTGCCCATCCTGGTCGAGCTGGACGGCGACGTAAACGGCCACAAGTTCA |
| | | GCGTGTCTGGCGAGGGCGAGGGCGATGCCACCTACGGCAAGCTGACCCT |
| | | GAAGTTCATCTGCACCACCGGCAAGCTGCCCGTGCCCTGGCCCACCCTCG |
| | | TGACCACCCTGACCTACGGCGTGCAGTGCTTCAGCCGCTACCCCGACCAC |
| | | ATGAAGCAGCACGACTTCTTCAAGTCCGCCATGCCCGAAGGCTACGTCCA |
| | | GGAGCGCACCATCTTCTTCAAGGACGACGGCAACTACAAGACCCGCGCC |
| | | GAGGTGAAGTTCGAGGGCGACACCCTGGTGAACCGCATCGAGCTGAAGG |
| | | GCATCGACTTCAAGGAGGACGGCAACATCCTGGGGCACAAGCTGGAGTA |
| | | CAACTACAACAGCCACAACGTCTATATCATGGCCGACAAGCAGAAGAAC |
| | | GGCATCAAGGCGAACTTCAAGATCCGCCACAACATCGAGGACGGCAGCG |
| | | TGCAGCTCGCCGACCACTACCAGCAGAACACCCCCATCGGCGACGGCCCC |
| | | GTGCTGCTGCCCGACAACCACTACCTGAGCACCCAGTCCGCCCTGAGCAA |
| | | AGACCCCAACGAGAAGCGCGATCACATGGTCCTGCTGGAGTTCGTGACC |
| | | GCCGCCGGGATCACTCTCGGCATGGACGAGCTGTACAAGTAATGAAAGC |
| | | TTTCGACAATCAACCTCTGGATTACAAAATTTGTGAAAGATTGACTGGTA |
| | | TTCTTAACTATGTTGCTCCTTTTACGCTATGTGGATACGCTGCTTTAATGC |
| | | CTTTGTATCATGCTATTGCTTCCCGTATGGCTTTCATTTTCTCCTCCTTGTA |
| | | TAAATCCTGGTTGCTGTCTCTTTATGAGGAGTTGTGGCCCGTTGTCAGGCA |
| | | ACGTGGCGTGGTGTGCACTGTGTTTGCTGACGCAACCCCCACTGGTTGGG |
| | | GCATTGCCACCACCTGTCAGCTCCTTTCCGGGACTTTCGCTTTCCCCCTCC |
| | | CTATTGCCACGGCGGAACTCATCGCCGCCTGCCTTGCCCGCTGCTGGACA |
| | | GGGGCTCGGCTGTTGGGCACTGACAATTCCGTGGTGTTGTCGGGGAAGCT |
| | | GACGTCCTTTCCATGGCTGCTCGCCTGTGTTGCCACCTGGATTCTGCGCGG |
| | | GACGTCCTTCTGCTACGTCCCTTCGGCCCTCAATCCAGCGGACCTTCCTTC |
| | | CCGCGGCCTGCTGCCGGCTCTGCGGCCTCTTCCGCGTCTTCGCCTTCGCCC |
| | | TCAGACGAGTCGGATCTCCCTTTGGGCCGCCTCCCCGCCTGGAGTCGACT |
| | | GCTTTATTTGTGAAATTTGTGATGCTATTGCTTTATTTGTAACCATTATAA |
| | | GCTGCAATAAACAAGTTAACAACAACAATTGCATTCATTTTATGTTTCAG |
| | | GTTCAGGGGGAGATGTGGGAGGTTTTTTAAAGCACTAGTGTGAGGCCCTG |
| | | GGCCCAGGATGGGGCAGGCAGGGTGGGGTACCTGGACCTACAGGTGCCG |
| | | ACCTTTACTGTGGCACTGGGCGGGAGGGGGGCTGGCTGGGGCACAGGAA |
| | | GTGGTTTCTGGGTCCCAGGCAAGTCTGTGACTTATGCAGATGTTGCAGGG |
| | | CCAAGAAAATCCCCACCTGCCAGGCCTCAGAGATTGGAGGCTCTCCCCGA |
| | | CCTCCCAATCCCTGTCTCAGGAGAGGAGGAGGCCGTGGATCCTACGTAGA |
| | | TAAGTAGCATGGCGGGTTAATCATTAACTACAAGGAACCCCTAGTGATGG |
| | | AGTTGGCCACTCCCTCTCTGCGCGCTCGCTCGCTCACTGAGGCCGGGCGA |
| | | CCAAAGGTCGCCCGACGCCCGGGCTTTGCCCGGGCGGCCTCAGTGAGCG |
| | | AGCGAGCGCGCCAGCTGGCGTAATAGCGAAGAGGCCCGCACCGATCGCC |
| | | CTTCCCAACAGTTGCGCAGCCTGAATGGCGAATGGCGATTCCGTTGCAAT |
| | | GGCTGGCGGTAATATTGTTCTGGATATTACCAGCAAGGCCGATAGTTTGA |
| | | GTTCTTCTACTCAGGCAAGTGATGTTATTACTAATCAAAGAAGTATTGCG |
| | | ACAACGGTTAATTTGCGTGATGGACAGACTCTTTTACTCGGTGGCCTCAC |
| | | TGATTATAAAAACACTTCTCAGGATTCTGGCGTACCGTTCCTGTCTAAAAT |
| | | CCCTTTAATCGGCCTCCTGTTTAGCTCCCGCTCTGATTCTAACGAGGAAAG |
| | | CACGTTATACGTGCTCGTCAAAGCAACCATAGTACGCGCCCTGTAGCGGC |
| | | GCATTAAGCGCGGCGGGTGTGGTGGTTACGCGCAGCGTGACCGCTACACT |
| | | TGCCAGCGCCCTAGCGCCCGCTCCTTTCGCTTTCTTCCCTTCCTTTCTCGCC |
| | | ACGTTCGCCGGCTTTCCCCGTCAAGCTCTAAATCGGGGGCTCCCTTTAGG |
| | | GTTCCGATTTAGTGCTTTACGGCACCTCGACCCCAAAAAACTTGATTAGG |
| | | GTGATGGTTCACGTAGTGGGCCATCGCCCTGATAGACGGTTTTTCGCCCTT |
| | | TGACGTTGGAGTCCACGTTCTTTAATAGTGGACTCTTGTTCCAAACTGGA |
| | | ACAACACTCAACCCTATCTCGGTCTATTCTTTTGATTTATAAGGGATTTTG |
| | | CCGATTTCGGCCTATTGGTTAAAAAATGAGCTGATTTAACAAAAATTTAA |
| | | CGCGAATTTTAACAAAATATTAACGTTTACAATTTAAATATTTGCTTATAC |
| | | AATCTTCCTGTTTTTGGGGCTTTTCTGATTATCAACCGGGGTACATATGAT |
| | | TGACATGCTAGTTTTACGATTACCGTTCATCGATTCTCTTGTTTGCTCCAG |
| | | ACTCTCAGGCAATGACCTGATAGCCTTTGTAGAGACCTCTCAAAAATAGC |
| | | TACCCTCTCCGGCATGAATTTATCAGCTAGAACGGTTGAATATCATATTG |
| | | ATGGTGATTTGACTGTCTCCGGCCTTTCTCACCCGTTTGAATCTTTACCTA |
| | | CACATTACTCAGGCATTGCATTTAAAATATATGAGGGTTCTAAAAATTTTT |
| | | ATCCTTGCGTTGAAATAAAGGCTTCTCCCGCAAAAGTATTACAGGGTCAT |
| | | AATGTTTTTGGTACAACCGATTTAGCTTTATGCTCTGAGGCTTTATTGCTT |
| | | AATTTTGCTAATTCTTTGCCTTGCCTGTATGATTTATTGGATGTTGGAATC |
| | | GCCTGATGCGGTATTTTCTCCTTACGCATCTGTGCGGTATTTCACACCGCA |
| | | TATGGTGCACTCTCAGTACAATCTGCTCTGATGCCGCATAGTTAAGCCAG |
| | | CCCCGACACCCGCCAACACCCGCTGACGCGCCCTGACGGGCTTGTCTGCT |
| | | CCCGGCATCCGCTTACAGACAAGCTGTGACCGTCTCCGGGAGCTGCATGT |
| | | GTCAGAGGTTTTCACCGTCATCACCGAAACGCGCGAGACGAAAGGGCCT |
| | | CGTGATACGCCTATTTTTATAGGTTAATGTCATGATAATAATGGTTTCTTA |
| | | GACGTCAGGTGGCACTTTTCGGGGAAATGTGCGCGGAACCCCTATTTGTT |
| | | TATTTTTCTAAATACATTCAAATATGTATCCGCTCATGAGACAATAACCCT |
| | | GATAAATGCTTCAATAATATTGAAAAAGGAAGAGTATGAGTATTCAACAT |
| | | TTCCGTGTCGCCCTTATTCCCTTTTTTGCGGCATTTTGCCTTCCTGTTTTTG |
| | | CTCACCCAGAAACGCTGGTGAAAGTAAAAGATGCTGAAGATCAGTTGGG |
| | | TGCACGAGTGGGTTACATCGAACTGGATCTCAACAGCGGTAAGATCCTTG |
| | | AGAGTTTTCGCCCCGAAGAACGTTTTCCAATGATGAGCACTTTTAAAGTT |
| | | CTGCTATGTGGCGCGGTATTATCCCGTATTGACGCCGGGCAAGAGCAACT |
| | | CGGTCGCCGCATACACTATTCTCAGAATGACTTGGTTGAGTACTCACCAG |
| | | TCACAGAAAAGCATCTTACGGATGGCATGACAGTAAGAGAATTATGCAG |
| | | TGCTGCCATAACCATGAGTGATAACACTGCGGCCAACTTACTTCTGACAA |
| | | CGATCGGAGGACCGAAGGAGCTAACCGCTTTTTTGCACAACATGGGGGA |
| | | TCATGTAACTCGCCTTGATCGTTGGGAACCGGAGCTGAATGAAGCCATAC |
| | | CAAACGACGAGCGTGACACCACGATGCCTGTAGCAATGGCAACAACGTT |
| | | GCGCAAACTATTAACTGGCGAACTACTTACTCTAGCTTCCCGGCAACAAT |
| | | TAATAGACTGGATGGAGGCGGATAAAGTTGCAGGACCACTTCTGCGCTCG |
| | | GCCCTTCCGGCTGGCTGGTTTATTGCTGATAAATCTGGAGCCGGTGAGCG |
| | | TGGGTCTCGCGGTATCATTGCAGCACTGGGGCCAGATGGTAAGCCCTCCC |
| | | GTATCGTAGTTATCTACACGACGGGGAGTCAGGCAACTATGGATGAACG |
| | | AAATAGACAGATCGCTGAGATAGGTGCCTCACTGATTAAGCATTGGTAAC |
| | | TGTCAGACCAAGTTTACTCATATATACTTTAGATTGATTTAAAACTTCATT |
| | | TTTAATTTAAAAGGATCTAGGTGAAGATCCTTTTTGATAATCTCATGACCA |
| | | AAATCCCTTAACGTGAGTTTTCGTTCCACTGAGCGTCAGACCCC |
| 137 | #3019 | GTAGAAAAGATCAAAGGATCTTCTTGAGATCCTTTTTTTCTGCGCGTAATC |
| | pAAV_FOXP3. | TGCTGCTTGCAAACAAAAAAACCACCGCTACCAGCGGTGGTTTGTTTGCC |
| | 025_MND.FOX | GGATCAAGAGCTACCAACTCTTTTTCCGAAGGTAACTGGCTTCAGCAGAG |
| | P3geneartCDS.P | CGCAGATACCAAATACTGTCCTTCTAGTGTAGCCGTAGTTAGGCCACCAC |
| | 2A.GFP.WPREc | TTCAAGAACTCTGTAGCACCGCCTACATACCTCGCTCTGCTAATCCTGTTA |
| | 3.pA_025 | CCAGTGGCTGCTGCCAGTGGCGATAAGTCGTGTCTTACCGGGTTGGACTC |
| | | AAGACGATAGTTACCGGATAAGGCGCAGCGGTCGGGCTGAACGGGGGGT |
| | | TCGTGCACACAGCCCAGCTTGGAGCGAACGACCTACACCGAACTGAGAT |
| | | ACCTACAGCGTGAGCTATGAGAAAGCGCCACGCTTCCCGAAGGGAGAAA |
| | | GGCGGACAGGTATCCGGTAAGCGGCAGGGTCGGAACAGGAGAGCGCACG |
| | | AGGGAGCTTCCAGGGGGAAACGCCTGGTATCTTTATAGTCCTGTCGGGTT |
| | | TCGCCACCTCTGACTTGAGCGTCGATTTTTGTGATGCTCGTCAGGGGGGC |
| | | GGAGCCTATGGAAAAACGCCAGCAACGCGGCCTTTTTACGGTTCCTGGCC |
| | | TTTTGCTGGCCTTTTGCTCACATGTTCTTTCCTGCGTTATCCCCTGATTCTG |
| | | TGGATAACCGTATTACCGCCTTTGAGTGAGCTGATACCGCTCGCCGCAGC |
| | | CGAACGACCGAGCGCAGCGAGTCAGTGAGCGAGGAAGCGGAAGAGCGC |
| | | CCAATACGCAAACCGCCTCTCCCCGCGCGTTGGCCGATTCATTAATGCAG |
| | | CTGCGCGCTCGCTCGCTCACTGAGGCCGCCCGGGCAAAGCCCGGGCGTCG |
| | | GGCGACCTTTGGTCGCCCGGCCTCAGTGAGCGAGCGAGCGCGCAGAGAG |
| | | GGAGTGGCCAACTCCATCACTAGGGGTTCCTTGTAGTTAATGATTAACCC |
| | | GCCATGCTACTTATCTACGTAGCGGCCGCTGCTAGCGTGGGCAGGCAAGC |
| | | CAGGTGCTGGACCTCTGCACGTGGGGCATGTGTGGGTATGTACATGTACC |
| | | TGTGTTCTTGGTGTGTGTGTGTGTGTGTGTGTGTGTGTGTGTCTAGAGCTG |
| | | GGGTGCAACTATGGGGCCCCTCGGGACATGTCCCAGCCAATGCCTGCTTT |
| | | GACCAGAGGAGTGTCCACGTGGCTCAGGTGGTCGAGTATCTCATACCGCC |
| | | CTAGCACACGTGTGACTCCTTTCCCCTATTGTCTACACGCGTAGGAACAG |
| | | AGAAACAGGAGAATATGGGCCAAACAGGATATCTGTGGTAAGCAGTTCC |
| | | TGCCCCGGCTCAGGGCCAAGAACAGTTGGAACAGCAGAATATGGGCCAA |
| | | ACAGGATATCTGTGGTAAGCAGTTCCTGCCCCGGCTCAGGGCCAAGAACA |
| | | GATGGTCCCCAGATGCGGTCCCGCCCTCAGCAGTTTCTAGAGAACCATCA |
| | | GATGTTTCCAGGGTGCCCCAAGGACCTGAAATGACCCTGTGCCTTATTTG |
| | | AACTAACCAATCAGTTCGCTTCTCGCTTCTGTTCGCGCGCTTCTGCTCCCC |
| | | GAGCTCTATATAAGCAGAGCTCGTTTAGTGAACCGTCAGATCGCCTGGAG |
| | | ACGCCATCCACGCTGTTTTGACTTCCATAGAAGGATCTCGAGGCCACCAT |
| | | GCCTAATCCTCGGCCTGGAAAGCCTAGCGCTCCTTCTCTTGCTCTGGGACC |
| | | TTCTCCTGGCGCCTCTCCATCTTGGAGAGCCGCTCCTAAAGCCAGCGATCT |
| | | GCTGGGAGCTAGAGGACCTGGCGGCACATTTCAGGGCAGAGATCTTAGA |
| | | GGCGGAGCCCACGCTAGCTCCTCCAGCCTTAATCCTATGCCTCCTAGCCA |
| | | GCTCCAGCTGCCTACACTGCCTCTGGTTATGGTGGCTCCTAGCGGAGCTA |
| | | GACTGGGCCCTCTGCCTCATCTGCAAGCTCTGCTGCAGGACAGACCCCAC |
| | | TTCATGCACCAGCTGAGCACCGTGGATGCCCACGCAAGAACACCTGTGCT |
| | | GCAGGTTCACCCTCTGGAATCCCCAGCCATGATCAGCCTGACACCTCCAA |
| | | CAACAGCCACCGGCGTGTTCAGCCTGAAAGCCAGACCTGGACTGCCTCCT |
| | | GGCATCAATGTGGCCAGCCTGGAATGGGTGTCCAGAGAACCTGCTCTGCT |
| | | GTGCACATTCCCCAATCCAAGCGCTCCCAGAAAGGACAGCACACTGTCTG |
| | | CCGTGCCTCAGAGCAGCTATCCCCTGCTTGCTAACGGCGTGTGCAAGTGG |
| | | CCTGGATGCGAGAAGGTGTTCGAGGAACCCGAGGACTTCCTGAAGCACT |
| | | GCCAGGCCGATCATCTGCTGGACGAGAAAGGCAGAGCCCAGTGTCTGCT |
| | | CCAGCGCGAGATGGTGCAGTCTCTGGAACAGCAGCTGGTCCTGGAAAAA |
| | | GAAAAGCTGAGCGCCATGCAGGCCCACCTGGCCGGAAAAATGGCCCTGA |
| | | CAAAGGCCAGCAGCGTGGCCTCTTCTGATAAGGGCAGCTGCTGCATTGTG |
| | | GCCGCTGGATCTCAGGGACCTGTGGTTCCTGCTTGGAGCGGACCTAGAGA |
| | | GGCCCCTGATTCTCTGTTTGCCGTGCGGAGACACCTGTGGGGCTCTCACG |
| | | GCAACTCTACTTTCCCCGAGTTCCTGCACAACATGGACTACTTCAAGTTCC |
| | | ACAACATGCGGCCTCCATTCACCTACGCCACACTGATCAGATGGGCCATT |
| | | CTGGAAGCCCCTGAGAAGCAGAGAACCCTGAACGAGATCTACCACTGGT |
| | | TTACCCGGATGTTCGCCTTCTTCCGGAATCACCCTGCCACCTGGAAGAAC |
| | | GCCATCCGGCACAATCTGAGCCTGCACAAGTGCTTCGTGCGCGTGGAATC |
| | | TGAGAAAGGCGCCGTGTGGACAGTGGACGAGCTGGAATTCAGAAAGAAG |
| | | AGAAGCCAGCGGCCTAGCCGGTGCAGCAATCCTACACCTGGACCTGGAA |
| | | GCGGAGCGACTAACTTCAGCCTGCTGAAGCAGGCCGGAGATGTGGAGGA |
| | | AAACCCTGGACCGATGGTGAGCAAGGGCGAGGAGCTGTTCACCGGGGTG |
| | | GTGCCCATCCTGGTCGAGCTGGACGGCGACGTAAACGGCCACAAGTTCA |
| | | GCGTGTCTGGCGAGGGCGAGGGCGATGCCACCTACGGCAAGCTGACCCT |
| | | GAAGTTCATCTGCACCACCGGCAAGCTGCCCGTGCCCTGGCCCACCCTCG |
| | | TGACCACCCTGACCTACGGCGTGCAGTGCTTCAGCCGCTACCCCGACCAC |
| | | ATGAAGCAGCACGACTTCTTCAAGTCCGCCATGCCCGAAGGCTACGTCCA |
| | | GGAGCGCACCATCTTCTTCAAGGACGACGGCAACTACAAGACCCGCGCC |
| | | GAGGTGAAGTTCGAGGGCGACACCCTGGTGAACCGCATCGAGCTGAAGG |
| | | GCATCGACTTCAAGGAGGACGGCAACATCCTGGGGCACAAGCTGGAGTA |
| | | CAACTACAACAGCCACAACGTCTATATCATGGCCGACAAGCAGAAGAAC |
| | | GGCATCAAGGCGAACTTCAAGATCCGCCACAACATCGAGGACGGCAGCG |
| | | TGCAGCTCGCCGACCACTACCAGCAGAACACCCCCATCGGCGACGGCCCC |
| | | GTGCTGCTGCCCGACAACCACTACCTGAGCACCCAGTCCGCCCTGAGCAA |
| | | AGACCCCAACGAGAAGCGCGATCACATGGTCCTGCTGGAGTTCGTGACC |
| | | GCCGCCGGGATCACTCTCGGCATGGACGAGCTGTACAAGTAATGAAAGC |
| | | TTGATAATCAACCTCTGGATTACAAAATTTGTGAAAGATTGACTGGTATT |
| | | CTTAACTATGTTGCTCCTTTTACGCTATGTGGATACGCTGCTTTAATGCCT |
| | | TTGTATCATGCTATTGCTTCCCGTATGGCTTTCATTTTCTCCTCCTTGTATA |
| | | AATCCTGGTTAGTTCTTGCCACGGCGGAACTCATCGCCGCCTGCCTTGCCC |
| | | GCTGCTGGACAGGGGCTCGGCTGTTGGGCACTGACAATTCCGTGGGTCGA |
| | | CTGCTTTATTTGTGAAATTTGTGATGCTATTGCTTTATTTGTAACCATTATA |
| | | AGCTGCAATAAACAAGTTAACAACAACAATTGCATTCATTTTATGTTTCA |
| | | GGTTCAGGGGGAGATGTGGGAGGTTTTTTAAAGCACTAGTGTGAGGCCCT |
| | | GGGCCCAGGATGGGGCAGGCAGGGTGGGGTACCTGGACCTACAGGTGCC |
| | | GACCTTTACTGTGGCACTGGGCGGGAGGGGGGCTGGCTGGGGCACAGGA |
| | | AGTGGTTTCTGGGTCCCAGGCAAGTCTGTGACTTATGCAGATGTTGCAGG |
| | | GCCAAGAAAATCCCCACCTGCCAGGCCTCAGAGATTGGAGGCTCTCCCCG |
| | | ACCTCCCAATCCCTGTCTCAGGAGAGGAGGAGGCCGTGGATCCTACGTAG |
| | | ATAAGTAGCATGGCGGGTTAATCATTAACTACAAGGAACCCCTAGTGATG |
| | | GAGTTGGCCACTCCCTCTCTGCGCGCTCGCTCGCTCACTGAGGCCGGGCG |
| | | ACCAAAGGTCGCCCGACGCCCGGGCTTTGCCCGGGCGGCCTCAGTGAGC |
| | | GAGCGAGCGCGCCAGCTGGCGTAATAGCGAAGAGGCCCGCACCGATCGC |
| | | CCTTCCCAACAGTTGCGCAGCCTGAATGGCGAATGGCGATTCCGTTGCAA |
| | | TGGCTGGCGGTAATATTGTTCTGGATATTACCAGCAAGGCCGATAGTTTG |
| | | AGTTCTTCTACTCAGGCAAGTGATGTTATTACTAATCAAAGAAGTATTGC |
| | | GACAACGGTTAATTTGCGTGATGGACAGACTCTTTTACTCGGTGGCCTCA |
| | | CTGATTATAAAAACACTTCTCAGGATTCTGGCGTACCGTTCCTGTCTAAA |
| | | ATCCCTTTAATCGGCCTCCTGTTTAGCTCCCGCTCTGATTCTAACGAGGAA |
| | | AGCACGTTATACGTGCTCGTCAAAGCAACCATAGTACGCGCCCTGTAGCG |
| | | GCGCATTAAGCGCGGCGGGTGTGGTGGTTACGCGCAGCGTGACCGCTAC |
| | | ACTTGCCAGCGCCCTAGCGCCCGCTCCTTTCGCTTTCTTCCCTTCCTTTCTC |
| | | GCCACGTTCGCCGGCTTTCCCCGTCAAGCTCTAAATCGGGGGCTCCCTTTA |
| | | GGGTTCCGATTTAGTGCTTTACGGCACCTCGACCCCAAAAAACTTGATTA |
| | | GGGTGATGGTTCACGTAGTGGGCCATCGCCCTGATAGACGGTTTTTCGCC |
| | | CTTTGACGTTGGAGTCCACGTTCTTTAATAGTGGACTCTTGTTCCAAACTG |
| | | GAACAACACTCAACCCTATCTCGGTCTATTCTTTTGATTTATAAGGGATTT |
| | | TGCCGATTTCGGCCTATTGGTTAAAAAATGAGCTGATTTAACAAAAATTT |
| | | AACGCGAATTTTAACAAAATATTAACGTTTACAATTTAAATATTTGCTTAT |
| | | ACAATCTTCCTGTTTTTGGGGCTTTTCTGATTATCAACCGGGGTACATATG |
| | | ATTGACATGCTAGTTTTACGATTACCGTTCATCGATTCTCTTGTTTGCTCC |
| | | AGACTCTCAGGCAATGACCTGATAGCCTTTGTAGAGACCTCTCAAAAATA |
| | | GCTACCCTCTCCGGCATGAATTTATCAGCTAGAACGGTTGAATATCATAT |
| | | TGATGGTGATTTGACTGTCTCCGGCCTTTCTCACCCGTTTGAATCTTTACC |
| | | TACACATTACTCAGGCATTGCATTTAAAATATATGAGGGTTCTAAAAATT |
| | | TTTATCCTTGCGTTGAAATAAAGGCTTCTCCCGCAAAAGTATTACAGGGT |
| | | CATAATGTTTTTGGTACAACCGATTTAGCTTTATGCTCTGAGGCTTTATTG |
| | | CTTAATTTTGCTAATTCTTTGCCTTGCCTGTATGATTTATTGGATGTTGGA |
| | | ATCGCCTGATGCGGTATTTTCTCCTTACGCATCTGTGCGGTATTTCACACC |
| | | GCATATGGTGCACTCTCAGTACAATCTGCTCTGATGCCGCATAGTTAAGC |
| | | CAGCCCCGACACCCGCCAACACCCGCTGACGCGCCCTGACGGGCTTGTCT |
| | | GCTCCCGGCATCCGCTTACAGACAAGCTGTGACCGTCTCCGGGAGCTGCA |
| | | TGTGTCAGAGGTTTTCACCGTCATCACCGAAACGCGCGAGACGAAAGGG |
| | | CCTCGTGATACGCCTATTTTTATAGGTTAATGTCATGATAATAATGGTTTC |
| | | TTAGACGTCAGGTGGCACTTTTCGGGGAAATGTGCGCGGAACCCCTATTT |
| | | GTTTATTTTTCTAAATACATTCAAATATGTATCCGCTCATGAGACAATAAC |
| | | CCTGATAAATGCTTCAATAATATTGAAAAAGGAAGAGTATGAGTATTCAA |
| | | CATTTCCGTGTCGCCCTTATTCCCTTTTTTGCGGCATTTTGCCTTCCTGTTT |
| | | TTGCTCACCCAGAAACGCTGGTGAAAGTAAAAGATGCTGAAGATCAGTT |
| | | GGGTGCACGAGTGGGTTACATCGAACTGGATCTCAACAGCGGTAAGATC |
| | | CTTGAGAGTTTTCGCCCCGAAGAACGTTTTCCAATGATGAGCACTTTTAA |
| | | AGTTCTGCTATGTGGCGCGGTATTATCCCGTATTGACGCCGGGCAAGAGC |
| | | AACTCGGTCGCCGCATACACTATTCTCAGAATGACTTGGTTGAGTACTCA |
| | | CCAGTCACAGAAAAGCATCTTACGGATGGCATGACAGTAAGAGAATTAT |
| | | GCAGTGCTGCCATAACCATGAGTGATAACACTGCGGCCAACTTACTTCTG |
| | | ACAACGATCGGAGGACCGAAGGAGCTAACCGCTTTTTTGCACAACATGG |
| | | GGGATCATGTAACTCGCCTTGATCGTTGGGAACCGGAGCTGAATGAAGCC |
| | | ATACCAAACGACGAGCGTGACACCACGATGCCTGTAGCAATGGCAACAA |
| | | CGTTGCGCAAACTATTAACTGGCGAACTACTTACTCTAGCTTCCCGGCAA |
| | | CAATTAATAGACTGGATGGAGGCGGATAAAGTTGCAGGACCACTTCTGC |
| | | GCTCGGCCCTTCCGGCTGGCTGGTTTATTGCTGATAAATCTGGAGCCGGT |
| | | GAGCGTGGGTCTCGCGGTATCATTGCAGCACTGGGGCCAGATGGTAAGCC |
| | | CTCCCGTATCGTAGTTATCTACACGACGGGGAGTCAGGCAACTATGGATG |
| | | AACGAAATAGACAGATCGCTGAGATAGGTGCCTCACTGATTAAGCATTG |
| | | GTAACTGTCAGACCAAGTTTACTCATATATACTTTAGATTGATTTAAAACT |
| | | TCATTTTTAATTTAAAAGGATCTAGGTGAAGATCCTTTTTGATAATCTCAT |
| | | GACCAAAATCCCTTAACGTGAGTTTTCGTTCCACTGAGCGTCAGACCCC |
| 138 | <#3020 | GTAGAAAAGATCAAAGGATCTTCTTGAGATCCTTTTTTTCTGCGCGTAATC |
| | pAAV_FOXP3. | TGCTGCTTGCAAACAAAAAAACCACCGCTACCAGCGGTGGTTTGTTTGCC |
| | 045_MND.FOX | GGATCAAGAGCTACCAACTCTTTTTCCGAAGGTAACTGGCTTCAGCAGAG |
| | P3geneartCDS.P | CGCAGATACCAAATACTGTCCTTCTAGTGTAGCCGTAGTTAGGCCACCAC |
| | 2A.LNGFR.WP | TTCAAGAACTCTGTAGCACCGCCTACATACCTCGCTCTGCTAATCCTGTTA |
| | RE3.pA_06 | CCAGTGGCTGCTGCCAGTGGCGATAAGTCGTGTCTTACCGGGTTGGACTC |
| | | AAGACGATAGTTACCGGATAAGGCGCAGCGGTCGGGCTGAACGGGGGGT |
| | | TCGTGCACACAGCCCAGCTTGGAGCGAACGACCTACACCGAACTGAGAT |
| | | ACCTACAGCGTGAGCTATGAGAAAGCGCCACGCTTCCCGAAGGGAGAAA |
| | | GGCGGACAGGTATCCGGTAAGCGGCAGGGTCGGAACAGGAGAGCGCACG |
| | | AGGGAGCTTCCAGGGGGAAACGCCTGGTATCTTTATAGTCCTGTCGGGTT |
| | | TCGCCACCTCTGACTTGAGCGTCGATTTTTGTGATGCTCGTCAGGGGGGC |
| | | GGAGCCTATGGAAAAACGCCAGCAACGCGGCCTTTTTACGGTTCCTGGCC |
| | | TTTTGCTGGCCTTTTGCTCACATGTTCTTTCCTGCGTTATCCCCTGATTCTG |
| | | TGGATAACCGTATTACCGCCTTTGAGTGAGCTGATACCGCTCGCCGCAGC |
| | | CGAACGACCGAGCGCAGCGAGTCAGTGAGCGAGGAAGCGGAAGAGCGC |
| | | CCAATACGCAAACCGCCTCTCCCCGCGCGTTGGCCGATTCATTAATGCAG |
| | | CTGCGCGCTCGCTCGCTCACTGAGGCCGCCCGGGCAAAGCCCGGGCGTCG |
| | | GGCGACCTTTGGTCGCCCGGCCTCAGTGAGCGAGCGAGCGCGCAGAGAG |
| | | GGAGTGGCCAACTCCATCACTAGGGGTTCCTTGTAGTTAATGATTAACCC |
| | | GCCATGCTACTTATCTACGTAGCGGCCGCAGTCCATGCCTAGTCACTGGG |
| | | GCAAAATAGGACTCCGAGGAGAAAGTCCGAGACCAGCTCCGGCAAGATG |
| | | AGCAAACACAGCCTGTGCAGGGTGCAGGGAGGGCTAGAGGCCTGAGGCT |
| | | TGAAACAGCTCTCAAGTGGAGGGGGAAACAACCATTGCCCTCATAGAGG |
| | | ACACATCCACACCAGGGCTGTGCTAGCGTGGGCAGGCAAGCCAGGTGCT |
| | | GGACCTCTGCACGTGGGGCATGTGTGGGTATGTACATGTACCTGTGTTCT |
| | | TGGTGTGTGTGTGTGTGTGTGTGTGTGTGTGTGTCTAGAGCTGGGGTGCA |
| | | ACTATGGGGCCCCTCGGGACATGTCCCAGCCAATGCCTGCTTTGACCAGA |
| | | GGAGTGTCCACGTGGCTCAGGTGGTCGAGTATCTCATACCGCCCTAGCAC |
| | | ACGTGTGACTCCTTTCCCCTATTGTCTACACGCGTAGGAACAGAGAAACA |
| | | GGAGAATATGGGCCAAACAGGATATCTGTGGTAAGCAGTTCCTGCCCCG |
| | | GCTCAGGGCCAAGAACAGTTGGAACAGCAGAATATGGGCCAAACAGGAT |
| | | ATCTGTGGTAAGCAGTTCCTGCCCCGGCTCAGGGCCAAGAACAGATGGTC |
| | | CCCAGATGCGGTCCCGCCCTCAGCAGTTTCTAGAGAACCATCAGATGTTT |
| | | CCAGGGTGCCCCAAGGACCTGAAATGACCCTGTGCCTTATTTGAACTAAC |
| | | CAATCAGTTCGCTTCTCGCTTCTGTTCGCGCGCTTCTGCTCCCCGAGCTCT |
| | | ATATAAGCAGAGCTCGTTTAGTGAACCGTCAGATCGCCTGGAGACGCCAT |
| | | CCACGCTGTTTTGACTTCCATAGAAGGATCTCGAGGCCACCATGCCTAAT |
| | | CCTCGGCCTGGAAAGCCTAGCGCTCCTTCTCTTGCTCTGGGACCTTCTCCT |
| | | GGCGCCTCTCCATCTTGGAGAGCCGCTCCTAAAGCCAGCGATCTGCTGGG |
| | | AGCTAGAGGACCTGGCGGCACATTTCAGGGCAGAGATCTTAGAGGCGGA |
| | | GCCCACGCTAGCTCCTCCAGCCTTAATCCTATGCCTCCTAGCCAGCTCCAG |
| | | CTGCCTACACTGCCTCTGGTTATGGTGGCTCCTAGCGGAGCTAGACTGGG |
| | | CCCTCTGCCTCATCTGCAAGCTCTGCTGCAGGACAGACCCCACTTCATGC |
| | | ACCAGCTGAGCACCGTGGATGCCCACGCAAGAACACCTGTGCTGCAGGTT |
| | | CACCCTCTGGAATCCCCAGCCATGATCAGCCTGACACCTCCAACAACAGC |
| | | CACCGGCGTGTTCAGCCTGAAAGCCAGACCTGGACTGCCTCCTGGCATCA |
| | | ATGTGGCCAGCCTGGAATGGGTGTCCAGAGAACCTGCTCTGCTGTGCACA |
| | | TTCCCCAATCCAAGCGCTCCCAGAAAGGACAGCACACTGTCTGCCGTGCC |
| | | TCAGAGCAGCTATCCCCTGCTTGCTAACGGCGTGTGCAAGTGGCCTGGAT |
| | | GCGAGAAGGTGTTCGAGGAACCCGAGGACTTCCTGAAGCACTGCCAGGC |
| | | CGATCATCTGCTGGACGAGAAAGGCAGAGCCCAGTGTCTGCTCCAGCGC |
| | | GAGATGGTGCAGTCTCTGGAACAGCAGCTGGTCCTGGAAAAAGAAAAGC |
| | | TGAGCGCCATGCAGGCCCACCTGGCCGGAAAAATGGCCCTGACAAAGGC |
| | | CAGCAGCGTGGCCTCTTCTGATAAGGGCAGCTGCTGCATTGTGGCCGCTG |
| | | GATCTCAGGGACCTGTGGTTCCTGCTTGGAGCGGACCTAGAGAGGCCCCT |
| | | GATTCTCTGTTTGCCGTGCGGAGACACCTGTGGGGCTCTCACGGCAACTC |
| | | TACTTTCCCCGAGTTCCTGCACAACATGGACTACTTCAAGTTCCACAACAT |
| | | GCGGCCTCCATTCACCTACGCCACACTGATCAGATGGGCCATTCTGGAAG |
| | | CCCCTGAGAAGCAGAGAACCCTGAACGAGATCTACCACTGGTTTACCCGG |
| | | ATGTTCGCCTTCTTCCGGAATCACCCTGCCACCTGGAAGAACGCCATCCG |
| | | GCACAATCTGAGCCTGCACAAGTGCTTCGTGCGCGTGGAATCTGAGAAAG |
| | | GCGCCGTGTGGACAGTGGACGAGCTGGAATTCAGAAAGAAGAGAAGCCA |
| | | GCGGCCTAGCCGGTGCAGCAATCCTACACCTGGACCTGGAAGCGGAGCG |
| | | ACTAACTTCAGCCTGCTGAAGCAGGCCGGAGATGTGGAGGAAAACCCTG |
| | | GACCGATGGGGGCAGGTGCCACCGGACGAGCCATGGACGGGCCGCGCCT |
| | | GCTGCTGTTGCTGCTTCTGGGGGTGTCCCTTGGAGGTGCCAAGGAGGCAT |
| | | GCCCCACAGGCCTGTACACACACAGCGGTGAGTGCTGCAAAGCCTGCAA |
| | | CCTGGGCGAGGGTGTGGCCCAGCCTTGTGGAGCCAACCAGACCGTGTGTG |
| | | AGCCCTGCCTGGACAGCGTGACGTTCTCCGACGTGGTGAGCGCGACCGAG |
| | | CCGTGCAAGCCGTGCACCGAGTGCGTGGGGCTCCAGAGCATGTCGGCGC |
| | | CGTGCGTGGAGGCCGACGACGCCGTGTGCCGCTGCGCCTACGGCTACTAC |
| | | CAGGATGAGACGACTGGGCGCTGCGAGGCGTGCCGCGTGTGCGAGGCGG |
| | | GCTCGGGCCTCGTGTTCTCCTGCCAGGACAAGCAGAACACCGTGTGCGAG |
| | | GAGTGCCCCGACGGCACGTATTCCGACGAGGCCAACCACGTGGACCCGT |
| | | GCCTGCCCTGCACCGTGTGCGAGGACACCGAGCGCCAGCTCCGCGAGTGC |
| | | ACACGCTGGGCCGACGCCGAGTGCGAGGAGATCCCTGGCCGTTGGATTA |
| | | CACGGTCCACACCCCCAGAGGGCTCGGACAGCACAGCCCCCAGCACCCA |
| | | GGAGCCTGAGGCACCTCCAGAACAAGACCTCATAGCCAGCACGGTGGCA |
| | | GGTGTGGTGACCACAGTGATGGGCAGCTCCCAGCCCGTGGTGACCCGAG |
| | | GCACCACCGACAACCTCATCCCTGTCTATTGCTCCATCCTGGCTGCTGTGG |
| | | TTGTGGGTCTTGTGGCCTACATAGCCTTCAAGAGGTGAAAGCTTCCACGG |
| | | AATTGTCAGTGCCCAACAGCCGAGCCCCTGTCCAGCAGCGGGCAAGGCA |
| | | GGCGGCGATGAGTTCCGCCGTGGCAAGAACTAACCAGGATTTATACAAG |
| | | GAGGAGAAAATGAAAGCCATACGGGAAGCAATAGCATGATACAAAGGC |
| | | ATTAAAGCAGCGTATCCACATAGCGTAAAAGGAGCAACATAGTTAAGAA |
| | | TACCAGTCAATCTTTCACAAATTTTGTAATCCAGAGGTTGATTATCGTCGA |
| | | CTGCTTTATTTGTGAAATTTGTGATGCTATTGCTTTATTTGTAACCATTATA |
| | | AGCTGCAATAAACAAGTTAACAACAACAATTGCATTCATTTTATGTTTCA |
| | | GGTTCAGGGGGAGATGTGGGAGGTTTTTTAAAGCACTAGTGTGAGGCCCT |
| | | GGGCCCAGGATGGGGCAGGCAGGGTGGGGTACCTGGACCTACAGGTGCC |
| | | GACCTTTACTGTGGCACTGGGCGGGAGGGGGGCTGGCTGGGGCACAGGA |
| | | AGTGGTTTCTGGGTCCCAGGCAAGTCTGTGACTTATGCAGATGTTGCAGG |
| | | GCCAAGAAAATCCCCACCTGCCAGGCCTCAGAGATTGGAGGCTCTCCCCG |
| | | ACCTCCCAATCCCTGTCTCAGGAGAGGAGGAGGCCGTATTGTAGTCCCAT |
| | | GAGCATAGCTATGTGTCCCCATCCCCATGTGACAAGAGAAGAGGACTGG |
| | | GGCCAAGTAGGTGAGGTGACAGGGCTGAGGCCAGCTCTGCAACTTATTA |
| | | GCTGTTTGATCTTTAAAAAGTTACTCGATCTCCATGAGCCTCAGTTTCCAT |
| | | ACGTGTAAAAGGGGGATGATCATAGCATCTACCATGTGGGCTTGCAGTGC |
| | | AGAGTATTTGAATTAGACACAGAACAGTGAGGATCAGGATGGCCTCTCA |
| | | CCCACCTGCCTTTCTGCCCAGCTGCCCACACTGCCCCTAGTCATGGTGGCA |
| | | CCCTCCGGGGCACGGCTGGGCCCCTTGCCCCACTTACAGGCACCGCGGCG |
| | | CTACGTAGATAAGTAGCATGGCGGGTTAATCATTAACTACAAGGAACCCC |
| | | TAGTGATGGAGTTGGCCACTCCCTCTCTGCGCGCTCGCTCGCTCACTGAG |
| | | GCCGGGCGACCAAAGGTCGCCCGACGCCCGGGCTTTGCCCGGGCGGCCT |
| | | CAGTGAGCGAGCGAGCGCGCCAGCTGGCGTAATAGCGAAGAGGCCCGCA |
| | | CCGATCGCCCTTCCCAACAGTTGCGCAGCCTGAATGGCGAATGGCGATTC |
| | | CGTTGCAATGGCTGGCGGTAATATTGTTCTGGATATTACCAGCAAGGCCG |
| | | ATAGTTTGAGTTCTTCTACTCAGGCAAGTGATGTTATTACTAATCAAAGA |
| | | AGTATTGCGACAACGGTTAATTTGCGTGATGGACAGACTCTTTTACTCGG |
| | | TGGCCTCACTGATTATAAAAACACTTCTCAGGATTCTGGCGTACCGTTCCT |
| | | GTCTAAAATCCCTTTAATCGGCCTCCTGTTTAGCTCCCGCTCTGATTCTAA |
| | | CGAGGAAAGCACGTTATACGTGCTCGTCAAAGCAACCATAGTACGCGCC |
| | | CTGTAGCGGCGCATTAAGCGCGGCGGGTGTGGTGGTTACGCGCAGCGTG |
| | | ACCGCTACACTTGCCAGCGCCCTAGCGCCCGCTCCTTTCGCTTTCTTCCCT |
| | | TCCTTTCTCGCCACGTTCGCCGGCTTTCCCCGTCAAGCTCTAAATCGGGGG |
| | | CTCCCTTTAGGGTTCCGATTTAGTGCTTTACGGCACCTCGACCCCAAAAA |
| | | ACTTGATTAGGGTGATGGTTCACGTAGTGGGCCATCGCCCTGATAGACGG |
| | | TTTTTCGCCCTTTGACGTTGGAGTCCACGTTCTTTAATAGTGGACTCTTGT |
| | | TCCAAACTGGAACAACACTCAACCCTATCTCGGTCTATTCTTTTGATTTAT |
| | | AAGGGATTTTGCCGATTTCGGCCTATTGGTTAAAAAATGAGCTGATTTAA |
| | | CAAAAATTTAACGCGAATTTTAACAAAATATTAACGTTTACAATTTAAAT |
| | | ATTTGCTTATACAATCTTCCTGTTTTTGGGGCTTTTCTGATTATCAACCGG |
| | | GGTACATATGATTGACATGCTAGTTTTACGATTACCGTTCATCGATTCTCT |
| | | TGTTTGCTCCAGACTCTCAGGCAATGACCTGATAGCCTTTGTAGAGACCT |
| | | CTCAAAAATAGCTACCCTCTCCGGCATGAATTTATCAGCTAGAACGGTTG |
| | | AATATCATATTGATGGTGATTTGACTGTCTCCGGCCTTTCTCACCCGTTTG |
| | | AATCTTTACCTACACATTACTCAGGCATTGCATTTAAAATATATGAGGGTT |
| | | CTAAAAATTTTTATCCTTGCGTTGAAATAAAGGCTTCTCCCGCAAAAGTA |
| | | TTACAGGGTCATAATGTTTTTGGTACAACCGATTTAGCTTTATGCTCTGAG |
| | | GCTTTATTGCTTAATTTTGCTAATTCTTTGCCTTGCCTGTATGATTTATTGG |
| | | ATGTTGGAATCGCCTGATGCGGTATTTTCTCCTTACGCATCTGTGCGGTAT |
| | | TTCACACCGCATATGGTGCACTCTCAGTACAATCTGCTCTGATGCCGCAT |
| | | AGTTAAGCCAGCCCCGACACCCGCCAACACCCGCTGACGCGCCCTGACG |
| | | GGCTTGTCTGCTCCCGGCATCCGCTTACAGACAAGCTGTGACCGTCTCCG |
| | | GGAGCTGCATGTGTCAGAGGTTTTCACCGTCATCACCGAAACGCGCGAGA |
| | | CGAAAGGGCCTCGTGATACGCCTATTTTTATAGGTTAATGTCATGATAAT |
| | | AATGGTTTCTTAGACGTCAGGTGGCACTTTTCGGGGAAATGTGCGCGGAA |
| | | CCCCTATTTGTTTATTTTTCTAAATACATTCAAATATGTATCCGCTCATGA |
| | | GACAATAACCCTGATAAATGCTTCAATAATATTGAAAAAGGAAGAGTAT |
| | | GAGTATTCAACATTTCCGTGTCGCCCTTATTCCCTTTTTTGCGGCATTTTGC |
| | | CTTCCTGTTTTTGCTCACCCAGAAACGCTGGTGAAAGTAAAAGATGCTGA |
| | | AGATCAGTTGGGTGCACGAGTGGGTTACATCGAACTGGATCTCAACAGCG |
| | | GTAAGATCCTTGAGAGTTTTCGCCCCGAAGAACGTTTTCCAATGATGAGC |
| | | ACTTTTAAAGTTCTGCTATGTGGCGCGGTATTATCCCGTATTGACGCCGGG |
| | | CAAGAGCAACTCGGTCGCCGCATACACTATTCTCAGAATGACTTGGTTGA |
| | | GTACTCACCAGTCACAGAAAAGCATCTTACGGATGGCATGACAGTAAGA |
| | | GAATTATGCAGTGCTGCCATAACCATGAGTGATAACACTGCGGCCAACTT |
| | | ACTTCTGACAACGATCGGAGGACCGAAGGAGCTAACCGCTTTTTTGCACA |
| | | ACATGGGGGATCATGTAACTCGCCTTGATCGTTGGGAACCGGAGCTGAAT |
| | | GAAGCCATACCAAACGACGAGCGTGACACCACGATGCCTGTAGCAATGG |
| | | CAACAACGTTGCGCAAACTATTAACTGGCGAACTACTTACTCTAGCTTCC |
| | | CGGCAACAATTAATAGACTGGATGGAGGCGGATAAAGTTGCAGGACCAC |
| | | TTCTGCGCTCGGCCCTTCCGGCTGGCTGGTTTATTGCTGATAAATCTGGAG |
| | | CCGGTGAGCGTGGGTCTCGCGGTATCATTGCAGCACTGGGGCCAGATGGT |
| | | AAGCCCTCCCGTATCGTAGTTATCTACACGACGGGGAGTCAGGCAACTAT |
| | | GGATGAACGAAATAGACAGATCGCTGAGATAGGTGCCTCACTGATTAAG |
| | | CATTGGTAACTGTCAGACCAAGTTTACTCATATATACTTTAGATTGATTTA |
| | | AAACTTCATTTTTAATTTAAAAGGATCTAGGTGAAGATCCTTTTTGATAAT |
| | | CTCATGACCAAAATCCCTTAACGTGAGTTTTCGTTCCACTGAGCGTCAGA |
| | | CCCC |
| 139 | <#3021 | GTAGAAAAGATCAAAGGATCTTCTTGAGATCCTTTTTTTCTGCGCGTAATC |
| | pAAV_FOXP3. | TGCTGCTTGCAAACAAAAAAACCACCGCTACCAGCGGTGGTTTGTTTGCC |
| | 025_MND.FOX | GGATCAAGAGCTACCAACTCTTTTTCCGAAGGTAACTGGCTTCAGCAGAG |
| | P3geneartCDS.P | CGCAGATACCAAATACTGTCCTTCTAGTGTAGCCGTAGTTAGGCCACCAC |
| | 2A.LNGFR.WP | TTCAAGAACTCTGTAGCACCGCCTACATACCTCGCTCTGCTAATCCTGTTA |
| | RE3.pA_025> | CCAGTGGCTGCTGCCAGTGGCGATAAGTCGTGTCTTACCGGGTTGGACTC |
| | | AAGACGATAGTTACCGGATAAGGCGCAGCGGTCGGGCTGAACGGGGGGT |
| | | TCGTGCACACAGCCCAGCTTGGAGCGAACGACCTACACCGAACTGAGAT |
| | | ACCTACAGCGTGAGCTATGAGAAAGCGCCACGCTTCCCGAAGGGAGAAA |
| | | GGCGGACAGGTATCCGGTAAGCGGCAGGGTCGGAACAGGAGAGCGCACG |
| | | AGGGAGCTTCCAGGGGGAAACGCCTGGTATCTTTATAGTCCTGTCGGGTT |
| | | TCGCCACCTCTGACTTGAGCGTCGATTTTTGTGATGCTCGTCAGGGGGGC |
| | | GGAGCCTATGGAAAAACGCCAGCAACGCGGCCTTTTTACGGTTCCTGGCC |
| | | TTTTGCTGGCCTTTTGCTCACATGTTCTTTCCTGCGTTATCCCCTGATTCTG |
| | | TGGATAACCGTATTACCGCCTTTGAGTGAGCTGATACCGCTCGCCGCAGC |
| | | CGAACGACCGAGCGCAGCGAGTCAGTGAGCGAGGAAGCGGAAGAGCGC |
| | | CCAATACGCAAACCGCCTCTCCCCGCGCGTTGGCCGATTCATTAATGCAG |
| | | CTGCGCGCTCGCTCGCTCACTGAGGCCGCCCGGGCAAAGCCCGGGCGTCG |
| | | GGCGACCTTTGGTCGCCCGGCCTCAGTGAGCGAGCGAGCGCGCAGAGAG |
| | | GGAGTGGCCAACTCCATCACTAGGGGTTCCTTGTAGTTAATGATTAACCC |
| | | GCCATGCTACTTATCTACGTAGCGGCCGCTGCTAGCGTGGGCAGGCAAGC |
| | | CAGGTGCTGGACCTCTGCACGTGGGGCATGTGTGGGTATGTACATGTACC |
| | | TGTGTTCTTGGTGTGTGTGTGTGTGTGTGTGTGTGTGTGTGTCTAGAGCTG |
| | | GGGTGCAACTATGGGGCCCCTCGGGACATGTCCCAGCCAATGCCTGCTTT |
| | | GACCAGAGGAGTGTCCACGTGGCTCAGGTGGTCGAGTATCTCATACCGCC |
| | | CTAGCACACGTGTGACTCCTTTCCCCTATTGTCTACACGCGTAGGAACAG |
| | | AGAAACAGGAGAATATGGGCCAAACAGGATATCTGTGGTAAGCAGTTCC |
| | | TGCCCCGGCTCAGGGCCAAGAACAGTTGGAACAGCAGAATATGGGCCAA |
| | | ACAGGATATCTGTGGTAAGCAGTTCCTGCCCCGGCTCAGGGCCAAGAACA |
| | | GATGGTCCCCAGATGCGGTCCCGCCCTCAGCAGTTTCTAGAGAACCATCA |
| | | GATGTTTCCAGGGTGCCCCAAGGACCTGAAATGACCCTGTGCCTTATTTG |
| | | AACTAACCAATCAGTTCGCTTCTCGCTTCTGTTCGCGCGCTTCTGCTCCCC |
| | | GAGCTCTATATAAGCAGAGCTCGTTTAGTGAACCGTCAGATCGCCTGGAG |
| | | ACGCCATCCACGCTGTTTTGACTTCCATAGAAGGATCTCGAGGCCACCAT |
| | | GCCTAATCCTCGGCCTGGAAAGCCTAGCGCTCCTTCTCTTGCTCTGGGACC |
| | | TTCTCCTGGCGCCTCTCCATCTTGGAGAGCCGCTCCTAAAGCCAGCGATCT |
| | | GCTGGGAGCTAGAGGACCTGGCGGCACATTTCAGGGCAGAGATCTTAGA |
| | | GGCGGAGCCCACGCTAGCTCCTCCAGCCTTAATCCTATGCCTCCTAGCCA |
| | | GCTCCAGCTGCCTACACTGCCTCTGGTTATGGTGGCTCCTAGCGGAGCTA |
| | | GACTGGGCCCTCTGCCTCATCTGCAAGCTCTGCTGCAGGACAGACCCCAC |
| | | TTCATGCACCAGCTGAGCACCGTGGATGCCCACGCAAGAACACCTGTGCT |
| | | GCAGGTTCACCCTCTGGAATCCCCAGCCATGATCAGCCTGACACCTCCAA |
| | | CAACAGCCACCGGCGTGTTCAGCCTGAAAGCCAGACCTGGACTGCCTCCT |
| | | GGCATCAATGTGGCCAGCCTGGAATGGGTGTCCAGAGAACCTGCTCTGCT |
| | | GTGCACATTCCCCAATCCAAGCGCTCCCAGAAAGGACAGCACACTGTCTG |
| | | CCGTGCCTCAGAGCAGCTATCCCCTGCTTGCTAACGGCGTGTGCAAGTGG |
| | | CCTGGATGCGAGAAGGTGTTCGAGGAACCCGAGGACTTCCTGAAGCACT |
| | | GCCAGGCCGATCATCTGCTGGACGAGAAAGGCAGAGCCCAGTGTCTGCT |
| | | CCAGCGCGAGATGGTGCAGTCTCTGGAACAGCAGCTGGTCCTGGAAAAA |
| | | GAAAAGCTGAGCGCCATGCAGGCCCACCTGGCCGGAAAAATGGCCCTGA |
| | | CAAAGGCCAGCAGCGTGGCCTCTTCTGATAAGGGCAGCTGCTGCATTGTG |
| | | GCCGCTGGATCTCAGGGACCTGTGGTTCCTGCTTGGAGCGGACCTAGAGA |
| | | GGCCCCTGATTCTCTGTTTGCCGTGCGGAGACACCTGTGGGGCTCTCACG |
| | | GCAACTCTACTTTCCCCGAGTTCCTGCACAACATGGACTACTTCAAGTTCC |
| | | ACAACATGCGGCCTCCATTCACCTACGCCACACTGATCAGATGGGCCATT |
| | | CTGGAAGCCCCTGAGAAGCAGAGAACCCTGAACGAGATCTACCACTGGT |
| | | TTACCCGGATGTTCGCCTTCTTCCGGAATCACCCTGCCACCTGGAAGAAC |
| | | GCCATCCGGCACAATCTGAGCCTGCACAAGTGCTTCGTGCGCGTGGAATC |
| | | TGAGAAAGGCGCCGTGTGGACAGTGGACGAGCTGGAATTCAGAAAGAAG |
| | | AGAAGCCAGCGGCCTAGCCGGTGCAGCAATCCTACACCTGGACCTGGAA |
| | | GCGGAGCGACTAACTTCAGCCTGCTGAAGCAGGCCGGAGATGTGGAGGA |
| | | AAACCCTGGACCGATGGGGGCAGGTGCCACCGGACGAGCCATGGACGGG |
| | | CCGCGCCTGCTGCTGTTGCTGCTTCTGGGGGTGTCCCTTGGAGGTGCCAA |
| | | GGAGGCATGCCCCACAGGCCTGTACACACACAGCGGTGAGTGCTGCAAA |
| | | GCCTGCAACCTGGGCGAGGGTGTGGCCCAGCCTTGTGGAGCCAACCAGA |
| | | CCGTGTGTGAGCCCTGCCTGGACAGCGTGACGTTCTCCGACGTGGTGAGC |
| | | GCGACCGAGCCGTGCAAGCCGTGCACCGAGTGCGTGGGGCTCCAGAGCA |
| | | TGTCGGCGCCGTGCGTGGAGGCCGACGACGCCGTGTGCCGCTGCGCCTAC |
| | | GGCTACTACCAGGATGAGACGACTGGGCGCTGCGAGGCGTGCCGCGTGT |
| | | GCGAGGCGGGCTCGGGCCTCGTGTTCTCCTGCCAGGACAAGCAGAACAC |
| | | CGTGTGCGAGGAGTGCCCCGACGGCACGTATTCCGACGAGGCCAACCAC |
| | | GTGGACCCGTGCCTGCCCTGCACCGTGTGCGAGGACACCGAGCGCCAGCT |
| | | CCGCGAGTGCACACGCTGGGCCGACGCCGAGTGCGAGGAGATCCCTGGC |
| | | CGTTGGATTACACGGTCCACACCCCCAGAGGGCTCGGACAGCACAGCCCC |
| | | CAGCACCCAGGAGCCTGAGGCACCTCCAGAACAAGACCTCATAGCCAGC |
| | | ACGGTGGCAGGTGTGGTGACCACAGTGATGGGCAGCTCCCAGCCCGTGG |
| | | TGACCCGAGGCACCACCGACAACCTCATCCCTGTCTATTGCTCCATCCTG |
| | | GCTGCTGTGGTTGTGGGTCTTGTGGCCTACATAGCCTTCAAGAGGTGAAA |
| | | GCTTCCACGGAATTGTCAGTGCCCAACAGCCGAGCCCCTGTCCAGCAGCG |
| | | GGCAAGGCAGGCGGCGATGAGTTCCGCCGTGGCAAGAACTAACCAGGAT |
| | | TTATACAAGGAGGAGAAAATGAAAGCCATACGGGAAGCAATAGCATGAT |
| | | ACAAAGGCATTAAAGCAGCGTATCCACATAGCGTAAAAGGAGCAACATA |
| | | GTTAAGAATACCAGTCAATCTTTCACAAATTTTGTAATCCAGAGGTTGAT |
| | | TATCGTCGACTGCTTTATTTGTGAAATTTGTGATGCTATTGCTTTATTTGTA |
| | | ACCATTATAAGCTGCAATAAACAAGTTAACAACAACAATTGCATTCATTT |
| | | TATGTTTCAGGTTCAGGGGGAGATGTGGGAGGTTTTTTAAAGCACTAGTG |
| | | TGAGGCCCTGGGCCCAGGATGGGGCAGGCAGGGTGGGGTACCTGGACCT |
| | | ACAGGTGCCGACCTTTACTGTGGCACTGGGCGGGAGGGGGGCTGGCTGG |
| | | GGCACAGGAAGTGGTTTCTGGGTCCCAGGCAAGTCTGTGACTTATGCAGA |
| | | TGTTGCAGGGCCAAGAAAATCCCCACCTGCCAGGCCTCAGAGATTGGAG |
| | | GCTCTCCCCGACCTCCCAATCCCTGTCTCAGGAGAGGAGGAGGCCGTGGA |
| | | TCCTACGTAGATAAGTAGCATGGCGGGTTAATCATTAACTACAAGGAACC |
| | | CCTAGTGATGGAGTTGGCCACTCCCTCTCTGCGCGCTCGCTCGCTCACTGA |
| | | GGCCGGGCGACCAAAGGTCGCCCGACGCCCGGGCTTTGCCCGGGCGGCC |
| | | TCAGTGAGCGAGCGAGCGCGCCAGCTGGCGTAATAGCGAAGAGGCCCGC |
| | | ACCGATCGCCCTTCCCAACAGTTGCGCAGCCTGAATGGCGAATGGCGATT |
| | | CCGTTGCAATGGCTGGCGGTAATATTGTTCTGGATATTACCAGCAAGGCC |
| | | GATAGTTTGAGTTCTTCTACTCAGGCAAGTGATGTTATTACTAATCAAAG |
| | | AAGTATTGCGACAACGGTTAATTTGCGTGATGGACAGACTCTTTTACTCG |
| | | GTGGCCTCACTGATTATAAAAACACTTCTCAGGATTCTGGCGTACCGTTC |
| | | CTGTCTAAAATCCCTTTAATCGGCCTCCTGTTTAGCTCCCGCTCTGATTCT |
| | | AACGAGGAAAGCACGTTATACGTGCTCGTCAAAGCAACCATAGTACGCG |
| | | CCCTGTAGCGGCGCATTAAGCGCGGCGGGTGTGGTGGTTACGCGCAGCGT |
| | | GACCGCTACACTTGCCAGCGCCCTAGCGCCCGCTCCTTTCGCTTTCTTCCC |
| | | TTCCTTTCTCGCCACGTTCGCCGGCTTTCCCCGTCAAGCTCTAAATCGGGG |
| | | GCTCCCTTTAGGGTTCCGATTTAGTGCTTTACGGCACCTCGACCCCAAAA |
| | | AACTTGATTAGGGTGATGGTTCACGTAGTGGGCCATCGCCCTGATAGACG |
| | | GTTTTTCGCCCTTTGACGTTGGAGTCCACGTTCTTTAATAGTGGACTCTTG |
| | | TTCCAAACTGGAACAACACTCAACCCTATCTCGGTCTATTCTTTTGATTTA |
| | | TAAGGGATTTTGCCGATTTCGGCCTATTGGTTAAAAAATGAGCTGATTTA |
| | | ACAAAAATTTAACGCGAATTTTAACAAAATATTAACGTTTACAATTTAAA |
| | | TATTTGCTTATACAATCTTCCTGTTTTTGGGGCTTTTCTGATTATCAACCGG |
| | | GGTACATATGATTGACATGCTAGTTTTACGATTACCGTTCATCGATTCTCT |
| | | TGTTTGCTCCAGACTCTCAGGCAATGACCTGATAGCCTTTGTAGAGACCT |
| | | CTCAAAAATAGCTACCCTCTCCGGCATGAATTTATCAGCTAGAACGGTTG |
| | | AATATCATATTGATGGTGATTTGACTGTCTCCGGCCTTTCTCACCCGTTTG |
| | | AATCTTTACCTACACATTACTCAGGCATTGCATTTAAAATATATGAGGGTT |
| | | CTAAAAATTTTTATCCTTGCGTTGAAATAAAGGCTTCTCCCGCAAAAGTA |
| | | TTACAGGGTCATAATGTTTTTGGTACAACCGATTTAGCTTTATGCTCTGAG |
| | | GCTTTATTGCTTAATTTTGCTAATTCTTTGCCTTGCCTGTATGATTTATTGG |
| | | ATGTTGGAATCGCCTGATGCGGTATTTTCTCCTTACGCATCTGTGCGGTAT |
| | | TTCACACCGCATATGGTGCACTCTCAGTACAATCTGCTCTGATGCCGCAT |
| | | AGTTAAGCCAGCCCCGACACCCGCCAACACCCGCTGACGCGCCCTGACG |
| | | GGCTTGTCTGCTCCCGGCATCCGCTTACAGACAAGCTGTGACCGTCTCCG |
| | | GGAGCTGCATGTGTCAGAGGTTTTCACCGTCATCACCGAAACGCGCGAGA |
| | | CGAAAGGGCCTCGTGATACGCCTATTTTTATAGGTTAATGTCATGATAAT |
| | | AATGGTTTCTTAGACGTCAGGTGGCACTTTTCGGGGAAATGTGCGCGGAA |
| | | CCCCTATTTGTTTATTTTTCTAAATACATTCAAATATGTATCCGCTCATGA |
| | | GACAATAACCCTGATAAATGCTTCAATAATATTGAAAAAGGAAGAGTAT |
| | | GAGTATTCAACATTTCCGTGTCGCCCTTATTCCCTTTTTTGCGGCATTTTGC |
| | | CTTCCTGTTTTTGCTCACCCAGAAACGCTGGTGAAAGTAAAAGATGCTGA |
| | | AGATCAGTTGGGTGCACGAGTGGGTTACATCGAACTGGATCTCAACAGCG |
| | | GTAAGATCCTTGAGAGTTTTCGCCCCGAAGAACGTTTTCCAATGATGAGC |
| | | ACTTTTAAAGTTCTGCTATGTGGCGCGGTATTATCCCGTATTGACGCCGGG |
| | | CAAGAGCAACTCGGTCGCCGCATACACTATTCTCAGAATGACTTGGTTGA |
| | | GTACTCACCAGTCACAGAAAAGCATCTTACGGATGGCATGACAGTAAGA |
| | | GAATTATGCAGTGCTGCCATAACCATGAGTGATAACACTGCGGCCAACTT |
| | | ACTTCTGACAACGATCGGAGGACCGAAGGAGCTAACCGCTTTTTTGCACA |
| | | ACATGGGGGATCATGTAACTCGCCTTGATCGTTGGGAACCGGAGCTGAAT |
| | | GAAGCCATACCAAACGACGAGCGTGACACCACGATGCCTGTAGCAATGG |
| | | CAACAACGTTGCGCAAACTATTAACTGGCGAACTACTTACTCTAGCTTCC |
| | | CGGCAACAATTAATAGACTGGATGGAGGCGGATAAAGTTGCAGGACCAC |
| | | TTCTGCGCTCGGCCCTTCCGGCTGGCTGGTTTATTGCTGATAAATCTGGAG |
| | | CCGGTGAGCGTGGGTCTCGCGGTATCATTGCAGCACTGGGGCCAGATGGT |
| | | AAGCCCTCCCGTATCGTAGTTATCTACACGACGGGGAGTCAGGCAACTAT |
| | | GGATGAACGAAATAGACAGATCGCTGAGATAGGTGCCTCACTGATTAAG |
| | | CATTGGTAACTGTCAGACCAAGTTTACTCATATATACTTTAGATTGATTTA |
| | | AAACTTCATTTTTAATTTAAAAGGATCTAGGTGAAGATCCTTTTTGATAAT |
| | | CTCATGACCAAAATCCCTTAACGTGAGTTTTCGTTCCACTGAGCGTCAGA |
| | | CCCC |
| 140 | <#3017 | GTAGAAAAGATCAAAGGATCTTCTTGAGATCCTTTTTTTCTGCGCGTAATC |
| | pAAV_FOXP3. | TGCTGCTTGCAAACAAAAAAACCACCGCTACCAGCGGTGGTTTGTTTGCC |
| | 025_MND.FOX | GGATCAAGAGCTACCAACTCTTTTTCCGAAGGTAACTGGCTTCAGCAGAG |
| | P3geneartCDS.P | CGCAGATACCAAATACTGTCCTTCTAGTGTAGCCGTAGTTAGGCCACCAC |
| | 2A.GFP.WPRE3 | TTCAAGAACTCTGTAGCACCGCCTACATACCTCGCTCTGCTAATCCTGTTA |
| | .pA_025> | CCAGTGGCTGCTGCCAGTGGCGATAAGTCGTGTCTTACCGGGTTGGACTC |
| | | AAGACGATAGTTACCGGATAAGGCGCAGCGGTCGGGCTGAACGGGGGGT |
| | | TCGTGCACACAGCCCAGCTTGGAGCGAACGACCTACACCGAACTGAGAT |
| | | ACCTACAGCGTGAGCTATGAGAAAGCGCCACGCTTCCCGAAGGGAGAAA |
| | | GGCGGACAGGTATCCGGTAAGCGGCAGGGTCGGAACAGGAGAGCGCACG |
| | | AGGGAGCTTCCAGGGGGAAACGCCTGGTATCTTTATAGTCCTGTCGGGTT |
| | | TCGCCACCTCTGACTTGAGCGTCGATTTTTGTGATGCTCGTCAGGGGGGC |
| | | GGAGCCTATGGAAAAACGCCAGCAACGCGGCCTTTTTACGGTTCCTGGCC |
| | | TTTTGCTGGCCTTTTGCTCACATGTTCTTTCCTGCGTTATCCCCTGATTCTG |
| | | TGGATAACCGTATTACCGCCTTTGAGTGAGCTGATACCGCTCGCCGCAGC |
| | | CGAACGACCGAGCGCAGCGAGTCAGTGAGCGAGGAAGCGGAAGAGCGC |
| | | CCAATACGCAAACCGCCTCTCCCCGCGCGTTGGCCGATTCATTAATGCAG |
| | | CTGCGCGCTCGCTCGCTCACTGAGGCCGCCCGGGCAAAGCCCGGGCGTCG |
| | | GGCGACCTTTGGTCGCCCGGCCTCAGTGAGCGAGCGAGCGCGCAGAGAG |
| | | GGAGTGGCCAACTCCATCACTAGGGGTTCCTTGTAGTTAATGATTAACCC |
| | | GCCATGCTACTTATCTACGTAGCGGCCGCTGCTAGCGTGGGCAGGCAAGC |
| | | CAGGTGCTGGACCTCTGCACGTGGGGCATGTGTGGGTATGTACATGTACC |
| | | TGTGTTCTTGGTGTGTGTGTGTGTGTGTGTGTGTGTGTGTGTCTAGAGCTG |
| | | GGGTGCAACTATGGGGCCCCTCGGGACATGTCCCAGCCAATGCCTGCTTT |
| | | GACCAGAGGAGTGTCCACGTGGCTCAGGTGGTCGAGTATCTCATACCGCC |
| | | CTAGCACACGTGTGACTCCTTTCCCCTATTGTCTACACGCGTAGGAACAG |
| | | AGAAACAGGAGAATATGGGCCAAACAGGATATCTGTGGTAAGCAGTTCC |
| | | TGCCCCGGCTCAGGGCCAAGAACAGTTGGAACAGCAGAATATGGGCCAA |
| | | ACAGGATATCTGTGGTAAGCAGTTCCTGCCCCGGCTCAGGGCCAAGAACA |
| | | GATGGTCCCCAGATGCGGTCCCGCCCTCAGCAGTTTCTAGAGAACCATCA |
| | | GATGTTTCCAGGGTGCCCCAAGGACCTGAAATGACCCTGTGCCTTATTTG |
| | | AACTAACCAATCAGTTCGCTTCTCGCTTCTGTTCGCGCGCTTCTGCTCCCC |
| | | GAGCTCTATATAAGCAGAGCTCGTTTAGTGAACCGTCAGATCGCCTGGAG |
| | | ACGCCATCCACGCTGTTTTGACTTCCATAGAAGGATCTCGAGGCCACCAT |
| | | GCCTAATCCTCGGCCTGGAAAGCCTAGCGCTCCTTCTCTTGCTCTGGGACC |
| | | TTCTCCTGGCGCCTCTCCATCTTGGAGAGCCGCTCCTAAAGCCAGCGATCT |
| | | GCTGGGAGCTAGAGGACCTGGCGGCACATTTCAGGGCAGAGATCTTAGA |
| | | GGCGGAGCCCACGCTAGCTCCTCCAGCCTTAATCCTATGCCTCCTAGCCA |
| | | GCTCCAGCTGCCTACACTGCCTCTGGTTATGGTGGCTCCTAGCGGAGCTA |
| | | GACTGGGCCCTCTGCCTCATCTGCAAGCTCTGCTGCAGGACAGACCCCAC |
| | | TTCATGCACCAGCTGAGCACCGTGGATGCCCACGCAAGAACACCTGTGCT |
| | | GCAGGTTCACCCTCTGGAATCCCCAGCCATGATCAGCCTGACACCTCCAA |
| | | CAACAGCCACCGGCGTGTTCAGCCTGAAAGCCAGACCTGGACTGCCTCCT |
| | | GGCATCAATGTGGCCAGCCTGGAATGGGTGTCCAGAGAACCTGCTCTGCT |
| | | GTGCACATTCCCCAATCCAAGCGCTCCCAGAAAGGACAGCACACTGTCTG |
| | | CCGTGCCTCAGAGCAGCTATCCCCTGCTTGCTAACGGCGTGTGCAAGTGG |
| | | CCTGGATGCGAGAAGGTGTTCGAGGAACCCGAGGACTTCCTGAAGCACT |
| | | GCCAGGCCGATCATCTGCTGGACGAGAAAGGCAGAGCCCAGTGTCTGCT |
| | | CCAGCGCGAGATGGTGCAGTCTCTGGAACAGCAGCTGGTCCTGGAAAAA |
| | | GAAAAGCTGAGCGCCATGCAGGCCCACCTGGCCGGAAAAATGGCCCTGA |
| | | CAAAGGCCAGCAGCGTGGCCTCTTCTGATAAGGGCAGCTGCTGCATTGTG |
| | | GCCGCTGGATCTCAGGGACCTGTGGTTCCTGCTTGGAGCGGACCTAGAGA |
| | | GGCCCCTGATTCTCTGTTTGCCGTGCGGAGACACCTGTGGGGCTCTCACG |
| | | GCAACTCTACTTTCCCCGAGTTCCTGCACAACATGGACTACTTCAAGTTCC |
| | | ACAACATGCGGCCTCCATTCACCTACGCCACACTGATCAGATGGGCCATT |
| | | CTGGAAGCCCCTGAGAAGCAGAGAACCCTGAACGAGATCTACCACTGGT |
| | | TTACCCGGATGTTCGCCTTCTTCCGGAATCACCCTGCCACCTGGAAGAAC |
| | | GCCATCCGGCACAATCTGAGCCTGCACAAGTGCTTCGTGCGCGTGGAATC |
| | | TGAGAAAGGCGCCGTGTGGACAGTGGACGAGCTGGAATTCAGAAAGAAG |
| | | AGAAGCCAGCGGCCTAGCCGGTGCAGCAATCCTACACCTGGACCTGGAA |
| | | GCGGAGCGACTAACTTCAGCCTGCTGAAGCAGGCCGGAGATGTGGAGGA |
| | | AAACCCTGGACCGATGGTGAGCAAGGGCGAGGAGCTGTTCACCGGGGTG |
| | | GTGCCCATCCTGGTCGAGCTGGACGGCGACGTAAACGGCCACAAGTTCA |
| | | GCGTGTCTGGCGAGGGCGAGGGCGATGCCACCTACGGCAAGCTGACCCT |
| | | GAAGTTCATCTGCACCACCGGCAAGCTGCCCGTGCCCTGGCCCACCCTCG |
| | | TGACCACCCTGACCTACGGCGTGCAGTGCTTCAGCCGCTACCCCGACCAC |
| | | ATGAAGCAGCACGACTTCTTCAAGTCCGCCATGCCCGAAGGCTACGTCCA |
| | | GGAGCGCACCATCTTCTTCAAGGACGACGGCAACTACAAGACCCGCGCC |
| | | GAGGTGAAGTTCGAGGGCGACACCCTGGTGAACCGCATCGAGCTGAAGG |
| | | GCATCGACTTCAAGGAGGACGGCAACATCCTGGGGCACAAGCTGGAGTA |
| | | CAACTACAACAGCCACAACGTCTATATCATGGCCGACAAGCAGAAGAAC |
| | | GGCATCAAGGCGAACTTCAAGATCCGCCACAACATCGAGGACGGCAGCG |
| | | TGCAGCTCGCCGACCACTACCAGCAGAACACCCCCATCGGCGACGGCCCC |
| | | GTGCTGCTGCCCGACAACCACTACCTGAGCACCCAGTCCGCCCTGAGCAA |
| | | AGACCCCAACGAGAAGCGCGATCACATGGTCCTGCTGGAGTTCGTGACC |
| | | GCCGCCGGGATCACTCTCGGCATGGACGAGCTGTACAAGTAATGAAAGC |
| | | TTCCACGGAATTGTCAGTGCCCAACAGCCGAGCCCCTGTCCAGCAGCGGG |
| | | CAAGGCAGGCGGCGATGAGTTCCGCCGTGGCAAGAACTAACCAGGATTT |
| | | ATACAAGGAGGAGAAAATGAAAGCCATACGGGAAGCAATAGCATGATAC |
| | | AAAGGCATTAAAGCAGCGTATCCACATAGCGTAAAAGGAGCAACATAGT |
| | | TAAGAATACCAGTCAATCTTTCACAAATTTTGTAATCCAGAGGTTGATTA |
| | | TCGTCGACTGCTTTATTTGTGAAATTTGTGATGCTATTGCTTTATTTGTAA |
| | | CCATTATAAGCTGCAATAAACAAGTTAACAACAACAATTGCATTCATTTT |
| | | ATGTTTCAGGTTCAGGGGGAGATGTGGGAGGTTTTTTAAAGCACTAGTGT |
| | | GAGGCCCTGGGCCCAGGATGGGGCAGGCAGGGTGGGGTACCTGGACCTA |
| | | CAGGTGCCGACCTTTACTGTGGCACTGGGCGGGAGGGGGGCTGGCTGGG |
| | | GCACAGGAAGTGGTTTCTGGGTCCCAGGCAAGTCTGTGACTTATGCAGAT |
| | | GTTGCAGGGCCAAGAAAATCCCCACCTGCCAGGCCTCAGAGATTGGAGG |
| | | CTCTCCCCGACCTCCCAATCCCTGTCTCAGGAGAGGAGGAGGCCGTGGAT |
| | | CCTACGTAGATAAGTAGCATGGCGGGTTAATCATTAACTACAAGGAACCC |
| | | CTAGTGATGGAGTTGGCCACTCCCTCTCTGCGCGCTCGCTCGCTCACTGA |
| | | GGCCGGGCGACCAAAGGTCGCCCGACGCCCGGGCTTTGCCCGGGCGGCC |
| | | TCAGTGAGCGAGCGAGCGCGCCAGCTGGCGTAATAGCGAAGAGGCCCGC |
| | | ACCGATCGCCCTTCCCAACAGTTGCGCAGCCTGAATGGCGAATGGCGATT |
| | | CCGTTGCAATGGCTGGCGGTAATATTGTTCTGGATATTACCAGCAAGGCC |
| | | GATAGTTTGAGTTCTTCTACTCAGGCAAGTGATGTTATTACTAATCAAAG |
| | | AAGTATTGCGACAACGGTTAATTTGCGTGATGGACAGACTCTTTTACTCG |
| | | GTGGCCTCACTGATTATAAAAACACTTCTCAGGATTCTGGCGTACCGTTC |
| | | CTGTCTAAAATCCCTTTAATCGGCCTCCTGTTTAGCTCCCGCTCTGATTCT |
| | | AACGAGGAAAGCACGTTATACGTGCTCGTCAAAGCAACCATAGTACGCG |
| | | CCCTGTAGCGGCGCATTAAGCGCGGCGGGTGTGGTGGTTACGCGCAGCGT |
| | | GACCGCTACACTTGCCAGCGCCCTAGCGCCCGCTCCTTTCGCTTTCTTCCC |
| | | TTCCTTTCTCGCCACGTTCGCCGGCTTTCCCCGTCAAGCTCTAAATCGGGG |
| | | GCTCCCTTTAGGGTTCCGATTTAGTGCTTTACGGCACCTCGACCCCAAAA |
| | | AACTTGATTAGGGTGATGGTTCACGTAGTGGGCCATCGCCCTGATAGACG |
| | | GTTTTTCGCCCTTTGACGTTGGAGTCCACGTTCTTTAATAGTGGACTCTTG |
| | | TTCCAAACTGGAACAACACTCAACCCTATCTCGGTCTATTCTTTTGATTTA |
| | | TAAGGGATTTTGCCGATTTCGGCCTATTGGTTAAAAAATGAGCTGATTTA |
| | | ACAAAAATTTAACGCGAATTTTAACAAAATATTAACGTTTACAATTTAAA |
| | | TATTTGCTTATACAATCTTCCTGTTTTTGGGGCTTTTCTGATTATCAACCGG |
| | | GGTACATATGATTGACATGCTAGTTTTACGATTACCGTTCATCGATTCTCT |
| | | TGTTTGCTCCAGACTCTCAGGCAATGACCTGATAGCCTTTGTAGAGACCT |
| | | CTCAAAAATAGCTACCCTCTCCGGCATGAATTTATCAGCTAGAACGGTTG |
| | | AATATCATATTGATGGTGATTTGACTGTCTCCGGCCTTTCTCACCCGTTTG |
| | | AATCTTTACCTACACATTACTCAGGCATTGCATTTAAAATATATGAGGGTT |
| | | CTAAAAATTTTTATCCTTGCGTTGAAATAAAGGCTTCTCCCGCAAAAGTA |
| | | TTACAGGGTCATAATGTTTTTGGTACAACCGATTTAGCTTTATGCTCTGAG |
| | | GCTTTATTGCTTAATTTTGCTAATTCTTTGCCTTGCCTGTATGATTTATTGG |
| | | ATGTTGGAATCGCCTGATGCGGTATTTTCTCCTTACGCATCTGTGCGGTAT |
| | | TTCACACCGCATATGGTGCACTCTCAGTACAATCTGCTCTGATGCCGCAT |
| | | AGTTAAGCCAGCCCCGACACCCGCCAACACCCGCTGACGCGCCCTGACG |
| | | GGCTTGTCTGCTCCCGGCATCCGCTTACAGACAAGCTGTGACCGTCTCCG |
| | | GGAGCTGCATGTGTCAGAGGTTTTCACCGTCATCACCGAAACGCGCGAGA |
| | | CGAAAGGGCCTCGTGATACGCCTATTTTTATAGGTTAATGTCATGATAAT |
| | | AATGGTTTCTTAGACGTCAGGTGGCACTTTTCGGGGAAATGTGCGCGGAA |
| | | CCCCTATTTGTTTATTTTTCTAAATACATTCAAATATGTATCCGCTCATGA |
| | | GACAATAACCCTGATAAATGCTTCAATAATATTGAAAAAGGAAGAGTAT |
| | | GAGTATTCAACATTTCCGTGTCGCCCTTATTCCCTTTTTTGCGGCATTTTGC |
| | | CTTCCTGTTTTTGCTCACCCAGAAACGCTGGTGAAAGTAAAAGATGCTGA |
| | | AGATCAGTTGGGTGCACGAGTGGGTTACATCGAACTGGATCTCAACAGCG |
| | | GTAAGATCCTTGAGAGTTTTCGCCCCGAAGAACGTTTTCCAATGATGAGC |
| | | ACTTTTAAAGTTCTGCTATGTGGCGCGGTATTATCCCGTATTGACGCCGGG |
| | | CAAGAGCAACTCGGTCGCCGCATACACTATTCTCAGAATGACTTGGTTGA |
| | | GTACTCACCAGTCACAGAAAAGCATCTTACGGATGGCATGACAGTAAGA |
| | | GAATTATGCAGTGCTGCCATAACCATGAGTGATAACACTGCGGCCAACTT |
| | | ACTTCTGACAACGATCGGAGGACCGAAGGAGCTAACCGCTTTTTTGCACA |
| | | ACATGGGGGATCATGTAACTCGCCTTGATCGTTGGGAACCGGAGCTGAAT |
| | | GAAGCCATACCAAACGACGAGCGTGACACCACGATGCCTGTAGCAATGG |
| | | CAACAACGTTGCGCAAACTATTAACTGGCGAACTACTTACTCTAGCTTCC |
| | | CGGCAACAATTAATAGACTGGATGGAGGCGGATAAAGTTGCAGGACCAC |
| | | TTCTGCGCTCGGCCCTTCCGGCTGGCTGGTTTATTGCTGATAAATCTGGAG |
| | | CCGGTGAGCGTGGGTCTCGCGGTATCATTGCAGCACTGGGGCCAGATGGT |
| | | AAGCCCTCCCGTATCGTAGTTATCTACACGACGGGGAGTCAGGCAACTAT |
| | | GGATGAACGAAATAGACAGATCGCTGAGATAGGTGCCTCACTGATTAAG |
| | | CATTGGTAACTGTCAGACCAAGTTTACTCATATATACTTTAGATTGATTTA |
| | | AAACTTCATTTTTAATTTAAAAGGATCTAGGTGAAGATCCTTTTTGATAAT |
| | | CTCATGACCAAAATCCCTTAACGTGAGTTTTCGTTCCACTGAGCGTCAGA |
| | | CCCC |
| 141 | <#3018_pAAV_ | GTAGAAAAGATCAAAGGATCTTCTTGAGATCCTTTTTTTCTGCGCGTAATC |
| | FOXP3.025_M | TGCTGCTTGCAAACAAAAAAACCACCGCTACCAGCGGTGGTTTGTTTGCC |
| | ND.FOXP3gene | GGATCAAGAGCTACCAACTCTTTTTCCGAAGGTAACTGGCTTCAGCAGAG |
| | artCDS.P2A.GF | CGCAGATACCAAATACTGTCCTTCTAGTGTAGCCGTAGTTAGGCCACCAC |
| | P.WPRE6.pA_0 | TTCAAGAACTCTGTAGCACCGCCTACATACCTCGCTCTGCTAATCCTGTTA |
| | 25> | CCAGTGGCTGCTGCCAGTGGCGATAAGTCGTGTCTTACCGGGTTGGACTC |
| | | AAGACGATAGTTACCGGATAAGGCGCAGCGGTCGGGCTGAACGGGGGGT |
| | | TCGTGCACACAGCCCAGCTTGGAGCGAACGACCTACACCGAACTGAGAT |
| | | ACCTACAGCGTGAGCTATGAGAAAGCGCCACGCTTCCCGAAGGGAGAAA |
| | | GGCGGACAGGTATCCGGTAAGCGGCAGGGTCGGAACAGGAGAGCGCACG |
| | | AGGGAGCTTCCAGGGGGAAACGCCTGGTATCTTTATAGTCCTGTCGGGTT |
| | | TCGCCACCTCTGACTTGAGCGTCGATTTTTGTGATGCTCGTCAGGGGGGC |
| | | GGAGCCTATGGAAAAACGCCAGCAACGCGGCCTTTTTACGGTTCCTGGCC |
| | | TTTTGCTGGCCTTTTGCTCACATGTTCTTTCCTGCGTTATCCCCTGATTCTG |
| | | TGGATAACCGTATTACCGCCTTTGAGTGAGCTGATACCGCTCGCCGCAGC |
| | | CGAACGACCGAGCGCAGCGAGTCAGTGAGCGAGGAAGCGGAAGAGCGC |
| | | CCAATACGCAAACCGCCTCTCCCCGCGCGTTGGCCGATTCATTAATGCAG |
| | | CTGCGCGCTCGCTCGCTCACTGAGGCCGCCCGGGCAAAGCCCGGGCGTCG |
| | | GGCGACCTTTGGTCGCCCGGCCTCAGTGAGCGAGCGAGCGCGCAGAGAG |
| | | GGAGTGGCCAACTCCATCACTAGGGGTTCCTTGTAGTTAATGATTAACCC |
| | | GCCATGCTACTTATCTACGTAGCGGCCGCTGCTAGCGTGGGCAGGCAAGC |
| | | CAGGTGCTGGACCTCTGCACGTGGGGCATGTGTGGGTATGTACATGTACC |
| | | TGTGTTCTTGGTGTGTGTGTGTGTGTGTGTGTGTGTGTGTGTCTAGAGCTG |
| | | GGGTGCAACTATGGGGCCCCTCGGGACATGTCCCAGCCAATGCCTGCTTT |
| | | GACCAGAGGAGTGTCCACGTGGCTCAGGTGGTCGAGTATCTCATACCGCC |
| | | CTAGCACACGTGTGACTCCTTTCCCCTATTGTCTACACGCGTAGGAACAG |
| | | AGAAACAGGAGAATATGGGCCAAACAGGATATCTGTGGTAAGCAGTTCC |
| | | TGCCCCGGCTCAGGGCCAAGAACAGTTGGAACAGCAGAATATGGGCCAA |
| | | ACAGGATATCTGTGGTAAGCAGTTCCTGCCCCGGCTCAGGGCCAAGAACA |
| | | GATGGTCCCCAGATGCGGTCCCGCCCTCAGCAGTTTCTAGAGAACCATCA |
| | | GATGTTTCCAGGGTGCCCCAAGGACCTGAAATGACCCTGTGCCTTATTTG |
| | | AACTAACCAATCAGTTCGCTTCTCGCTTCTGTTCGCGCGCTTCTGCTCCCC |
| | | GAGCTCTATATAAGCAGAGCTCGTTTAGTGAACCGTCAGATCGCCTGGAG |
| | | ACGCCATCCACGCTGTTTTGACTTCCATAGAAGGATCTCGAGGCCACCAT |
| | | GCCTAATCCTCGGCCTGGAAAGCCTAGCGCTCCTTCTCTTGCTCTGGGACC |
| | | TTCTCCTGGCGCCTCTCCATCTTGGAGAGCCGCTCCTAAAGCCAGCGATCT |
| | | GCTGGGAGCTAGAGGACCTGGCGGCACATTTCAGGGCAGAGATCTTAGA |
| | | GGCGGAGCCCACGCTAGCTCCTCCAGCCTTAATCCTATGCCTCCTAGCCA |
| | | GCTCCAGCTGCCTACACTGCCTCTGGTTATGGTGGCTCCTAGCGGAGCTA |
| | | GACTGGGCCCTCTGCCTCATCTGCAAGCTCTGCTGCAGGACAGACCCCAC |
| | | TTCATGCACCAGCTGAGCACCGTGGATGCCCACGCAAGAACACCTGTGCT |
| | | GCAGGTTCACCCTCTGGAATCCCCAGCCATGATCAGCCTGACACCTCCAA |
| | | CAACAGCCACCGGCGTGTTCAGCCTGAAAGCCAGACCTGGACTGCCTCCT |
| | | GGCATCAATGTGGCCAGCCTGGAATGGGTGTCCAGAGAACCTGCTCTGCT |
| | | GTGCACATTCCCCAATCCAAGCGCTCCCAGAAAGGACAGCACACTGTCTG |
| | | CCGTGCCTCAGAGCAGCTATCCCCTGCTTGCTAACGGCGTGTGCAAGTGG |
| | | CCTGGATGCGAGAAGGTGTTCGAGGAACCCGAGGACTTCCTGAAGCACT |
| | | GCCAGGCCGATCATCTGCTGGACGAGAAAGGCAGAGCCCAGTGTCTGCT |
| | | CCAGCGCGAGATGGTGCAGTCTCTGGAACAGCAGCTGGTCCTGGAAAAA |
| | | GAAAAGCTGAGCGCCATGCAGGCCCACCTGGCCGGAAAAATGGCCCTGA |
| | | CAAAGGCCAGCAGCGTGGCCTCTTCTGATAAGGGCAGCTGCTGCATTGTG |
| | | GCCGCTGGATCTCAGGGACCTGTGGTTCCTGCTTGGAGCGGACCTAGAGA |
| | | GGCCCCTGATTCTCTGTTTGCCGTGCGGAGACACCTGTGGGGCTCTCACG |
| | | GCAACTCTACTTTCCCCGAGTTCCTGCACAACATGGACTACTTCAAGTTCC |
| | | ACAACATGCGGCCTCCATTCACCTACGCCACACTGATCAGATGGGCCATT |
| | | CTGGAAGCCCCTGAGAAGCAGAGAACCCTGAACGAGATCTACCACTGGT |
| | | TTACCCGGATGTTCGCCTTCTTCCGGAATCACCCTGCCACCTGGAAGAAC |
| | | GCCATCCGGCACAATCTGAGCCTGCACAAGTGCTTCGTGCGCGTGGAATC |
| | | TGAGAAAGGCGCCGTGTGGACAGTGGACGAGCTGGAATTCAGAAAGAAG |
| | | AGAAGCCAGCGGCCTAGCCGGTGCAGCAATCCTACACCTGGACCTGGAA |
| | | GCGGAGCGACTAACTTCAGCCTGCTGAAGCAGGCCGGAGATGTGGAGGA |
| | | AAACCCTGGACCGATGGTGAGCAAGGGCGAGGAGCTGTTCACCGGGGTG |
| | | GTGCCCATCCTGGTCGAGCTGGACGGCGACGTAAACGGCCACAAGTTCA |
| | | GCGTGTCTGGCGAGGGCGAGGGCGATGCCACCTACGGCAAGCTGACCCT |
| | | GAAGTTCATCTGCACCACCGGCAAGCTGCCCGTGCCCTGGCCCACCCTCG |
| | | TGACCACCCTGACCTACGGCGTGCAGTGCTTCAGCCGCTACCCCGACCAC |
| | | ATGAAGCAGCACGACTTCTTCAAGTCCGCCATGCCCGAAGGCTACGTCCA |
| | | GGAGCGCACCATCTTCTTCAAGGACGACGGCAACTACAAGACCCGCGCC |
| | | GAGGTGAAGTTCGAGGGCGACACCCTGGTGAACCGCATCGAGCTGAAGG |
| | | GCATCGACTTCAAGGAGGACGGCAACATCCTGGGGCACAAGCTGGAGTA |
| | | CAACTACAACAGCCACAACGTCTATATCATGGCCGACAAGCAGAAGAAC |
| | | GGCATCAAGGCGAACTTCAAGATCCGCCACAACATCGAGGACGGCAGCG |
| | | TGCAGCTCGCCGACCACTACCAGCAGAACACCCCCATCGGCGACGGCCCC |
| | | GTGCTGCTGCCCGACAACCACTACCTGAGCACCCAGTCCGCCCTGAGCAA |
| | | AGACCCCAACGAGAAGCGCGATCACATGGTCCTGCTGGAGTTCGTGACC |
| | | GCCGCCGGGATCACTCTCGGCATGGACGAGCTGTACAAGTAATGAAAGC |
| | | TTTCGACAATCAACCTCTGGATTACAAAATTTGTGAAAGATTGACTGGTA |
| | | TTCTTAACTATGTTGCTCCTTTTACGCTATGTGGATACGCTGCTTTAATGC |
| | | CTTTGTATCATGCTATTGCTTCCCGTATGGCTTTCATTTTCTCCTCCTTGTA |
| | | TAAATCCTGGTTGCTGTCTCTTTATGAGGAGTTGTGGCCCGTTGTCAGGCA |
| | | ACGTGGCGTGGTGTGCACTGTGTTTGCTGACGCAACCCCCACTGGTTGGG |
| | | GCATTGCCACCACCTGTCAGCTCCTTTCCGGGACTTTCGCTTTCCCCCTCC |
| | | CTATTGCCACGGCGGAACTCATCGCCGCCTGCCTTGCCCGCTGCTGGACA |
| | | GGGGCTCGGCTGTTGGGCACTGACAATTCCGTGGTGTTGTCGGGGAAGCT |
| | | GACGTCCTTTCCATGGCTGCTCGCCTGTGTTGCCACCTGGATTCTGCGCGG |
| | | GACGTCCTTCTGCTACGTCCCTTCGGCCCTCAATCCAGCGGACCTTCCTTC |
| | | CCGCGGCCTGCTGCCGGCTCTGCGGCCTCTTCCGCGTCTTCGCCTTCGCCC |
| | | TCAGACGAGTCGGATCTCCCTTTGGGCCGCCTCCCCGCCTGGAGTCGACT |
| | | GCTTTATTTGTGAAATTTGTGATGCTATTGCTTTATTTGTAACCATTATAA |
| | | GCTGCAATAAACAAGTTAACAACAACAATTGCATTCATTTTATGTTTCAG |
| | | GTTCAGGGGGAGATGTGGGAGGTTTTTTAAAGCACTAGTGTGAGGCCCTG |
| | | GGCCCAGGATGGGGCAGGCAGGGTGGGGTACCTGGACCTACAGGTGCCG |
| | | ACCTTTACTGTGGCACTGGGCGGGAGGGGGGCTGGCTGGGGCACAGGAA |
| | | GTGGTTTCTGGGTCCCAGGCAAGTCTGTGACTTATGCAGATGTTGCAGGG |
| | | CCAAGAAAATCCCCACCTGCCAGGCCTCAGAGATTGGAGGCTCTCCCCGA |
| | | CCTCCCAATCCCTGTCTCAGGAGAGGAGGAGGCCGTGGATCCTACGTAGA |
| | | TAAGTAGCATGGCGGGTTAATCATTAACTACAAGGAACCCCTAGTGATGG |
| | | AGTTGGCCACTCCCTCTCTGCGCGCTCGCTCGCTCACTGAGGCCGGGCGA |
| | | CCAAAGGTCGCCCGACGCCCGGGCTTTGCCCGGGCGGCCTCAGTGAGCG |
| | | AGCGAGCGCGCCAGCTGGCGTAATAGCGAAGAGGCCCGCACCGATCGCC |
| | | CTTCCCAACAGTTGCGCAGCCTGAATGGCGAATGGCGATTCCGTTGCAAT |
| | | GGCTGGCGGTAATATTGTTCTGGATATTACCAGCAAGGCCGATAGTTTGA |
| | | GTTCTTCTACTCAGGCAAGTGATGTTATTACTAATCAAAGAAGTATTGCG |
| | | ACAACGGTTAATTTGCGTGATGGACAGACTCTTTTACTCGGTGGCCTCAC |
| | | TGATTATAAAAACACTTCTCAGGATTCTGGCGTACCGTTCCTGTCTAAAAT |
| | | CCCTTTAATCGGCCTCCTGTTTAGCTCCCGCTCTGATTCTAACGAGGAAAG |
| | | CACGTTATACGTGCTCGTCAAAGCAACCATAGTACGCGCCCTGTAGCGGC |
| | | GCATTAAGCGCGGCGGGTGTGGTGGTTACGCGCAGCGTGACCGCTACACT |
| | | TGCCAGCGCCCTAGCGCCCGCTCCTTTCGCTTTCTTCCCTTCCTTTCTCGCC |
| | | ACGTTCGCCGGCTTTCCCCGTCAAGCTCTAAATCGGGGGCTCCCTTTAGG |
| | | GTTCCGATTTAGTGCTTTACGGCACCTCGACCCCAAAAAACTTGATTAGG |
| | | GTGATGGTTCACGTAGTGGGCCATCGCCCTGATAGACGGTTTTTCGCCCTT |
| | | TGACGTTGGAGTCCACGTTCTTTAATAGTGGACTCTTGTTCCAAACTGGA |
| | | ACAACACTCAACCCTATCTCGGTCTATTCTTTTGATTTATAAGGGATTTTG |
| | | CCGATTTCGGCCTATTGGTTAAAAAATGAGCTGATTTAACAAAAATTTAA |
| | | CGCGAATTTTAACAAAATATTAACGTTTACAATTTAAATATTTGCTTATAC |
| | | AATCTTCCTGTTTTTGGGGCTTTTCTGATTATCAACCGGGGTACATATGAT |
| | | TGACATGCTAGTTTTACGATTACCGTTCATCGATTCTCTTGTTTGCTCCAG |
| | | ACTCTCAGGCAATGACCTGATAGCCTTTGTAGAGACCTCTCAAAAATAGC |
| | | TACCCTCTCCGGCATGAATTTATCAGCTAGAACGGTTGAATATCATATTG |
| | | ATGGTGATTTGACTGTCTCCGGCCTTTCTCACCCGTTTGAATCTTTACCTA |
| | | CACATTACTCAGGCATTGCATTTAAAATATATGAGGGTTCTAAAAATTTTT |
| | | ATCCTTGCGTTGAAATAAAGGCTTCTCCCGCAAAAGTATTACAGGGTCAT |
| | | AATGTTTTTGGTACAACCGATTTAGCTTTATGCTCTGAGGCTTTATTGCTT |
| | | AATTTTGCTAATTCTTTGCCTTGCCTGTATGATTTATTGGATGTTGGAATC |
| | | GCCTGATGCGGTATTTTCTCCTTACGCATCTGTGCGGTATTTCACACCGCA |
| | | TATGGTGCACTCTCAGTACAATCTGCTCTGATGCCGCATAGTTAAGCCAG |
| | | CCCCGACACCCGCCAACACCCGCTGACGCGCCCTGACGGGCTTGTCTGCT |
| | | CCCGGCATCCGCTTACAGACAAGCTGTGACCGTCTCCGGGAGCTGCATGT |
| | | GTCAGAGGTTTTCACCGTCATCACCGAAACGCGCGAGACGAAAGGGCCT |
| | | CGTGATACGCCTATTTTTATAGGTTAATGTCATGATAATAATGGTTTCTTA |
| | | GACGTCAGGTGGCACTTTTCGGGGAAATGTGCGCGGAACCCCTATTTGTT |
| | | TATTTTTCTAAATACATTCAAATATGTATCCGCTCATGAGACAATAACCCT |
| | | GATAAATGCTTCAATAATATTGAAAAAGGAAGAGTATGAGTATTCAACAT |
| | | TTCCGTGTCGCCCTTATTCCCTTTTTTGCGGCATTTTGCCTTCCTGTTTTTG |
| | | CTCACCCAGAAACGCTGGTGAAAGTAAAAGATGCTGAAGATCAGTTGGG |
| | | TGCACGAGTGGGTTACATCGAACTGGATCTCAACAGCGGTAAGATCCTTG |
| | | AGAGTTTTCGCCCCGAAGAACGTTTTCCAATGATGAGCACTTTTAAAGTT |
| | | CTGCTATGTGGCGCGGTATTATCCCGTATTGACGCCGGGCAAGAGCAACT |
| | | CGGTCGCCGCATACACTATTCTCAGAATGACTTGGTTGAGTACTCACCAG |
| | | TCACAGAAAAGCATCTTACGGATGGCATGACAGTAAGAGAATTATGCAG |
| | | TGCTGCCATAACCATGAGTGATAACACTGCGGCCAACTTACTTCTGACAA |
| | | CGATCGGAGGACCGAAGGAGCTAACCGCTTTTTTGCACAACATGGGGGA |
| | | TCATGTAACTCGCCTTGATCGTTGGGAACCGGAGCTGAATGAAGCCATAC |
| | | CAAACGACGAGCGTGACACCACGATGCCTGTAGCAATGGCAACAACGTT |
| | | GCGCAAACTATTAACTGGCGAACTACTTACTCTAGCTTCCCGGCAACAAT |
| | | TAATAGACTGGATGGAGGCGGATAAAGTTGCAGGACCACTTCTGCGCTCG |
| | | GCCCTTCCGGCTGGCTGGTTTATTGCTGATAAATCTGGAGCCGGTGAGCG |
| | | TGGGTCTCGCGGTATCATTGCAGCACTGGGGCCAGATGGTAAGCCCTCCC |
| | | GTATCGTAGTTATCTACACGACGGGGAGTCAGGCAACTATGGATGAACG |
| | | AAATAGACAGATCGCTGAGATAGGTGCCTCACTGATTAAGCATTGGTAAC |
| | | TGTCAGACCAAGTTTACTCATATATACTTTAGATTGATTTAAAACTTCATT |
| | | TTTAATTTAAAAGGATCTAGGTGAAGATCCTTTTTGATAATCTCATGACCA |
| | | AAATCCCTTAACGTGAGTTTTCGTTCCACTGAGCGTCAGACCCC |
| 142 | <#3019 | GTAGAAAAGATCAAAGGATCTTCTTGAGATCCTTTTTTTCTGCGCGTAATC |
| | pAAV_FOXP3. | TGCTGCTTGCAAACAAAAAAACCACCGCTACCAGCGGTGGTTTGTTTGCC |
| | 025_MND.FOX | GGATCAAGAGCTACCAACTCTTTTTCCGAAGGTAACTGGCTTCAGCAGAG |
| | P3geneartCDS.P | CGCAGATACCAAATACTGTCCTTCTAGTGTAGCCGTAGTTAGGCCACCAC |
| | 2A.GFP.WPREc | TTCAAGAACTCTGTAGCACCGCCTACATACCTCGCTCTGCTAATCCTGTTA |
| | 3.pA_025> | CCAGTGGCTGCTGCCAGTGGCGATAAGTCGTGTCTTACCGGGTTGGACTC |
| | | AAGACGATAGTTACCGGATAAGGCGCAGCGGTCGGGCTGAACGGGGGGT |
| | | TCGTGCACACAGCCCAGCTTGGAGCGAACGACCTACACCGAACTGAGAT |
| | | ACCTACAGCGTGAGCTATGAGAAAGCGCCACGCTTCCCGAAGGGAGAAA |
| | | GGCGGACAGGTATCCGGTAAGCGGCAGGGTCGGAACAGGAGAGCGCACG |
| | | AGGGAGCTTCCAGGGGGAAACGCCTGGTATCTTTATAGTCCTGTCGGGTT |
| | | TCGCCACCTCTGACTTGAGCGTCGATTTTTGTGATGCTCGTCAGGGGGGC |
| | | GGAGCCTATGGAAAAACGCCAGCAACGCGGCCTTTTTACGGTTCCTGGCC |
| | | TTTTGCTGGCCTTTTGCTCACATGTTCTTTCCTGCGTTATCCCCTGATTCTG |
| | | TGGATAACCGTATTACCGCCTTTGAGTGAGCTGATACCGCTCGCCGCAGC |
| | | CGAACGACCGAGCGCAGCGAGTCAGTGAGCGAGGAAGCGGAAGAGCGC |
| | | CCAATACGCAAACCGCCTCTCCCCGCGCGTTGGCCGATTCATTAATGCAG |
| | | CTGCGCGCTCGCTCGCTCACTGAGGCCGCCCGGGCAAAGCCCGGGCGTCG |
| | | GGCGACCTTTGGTCGCCCGGCCTCAGTGAGCGAGCGAGCGCGCAGAGAG |
| | | GGAGTGGCCAACTCCATCACTAGGGGTTCCTTGTAGTTAATGATTAACCC |
| | | GCCATGCTACTTATCTACGTAGCGGCCGCTGCTAGCGTGGGCAGGCAAGC |
| | | CAGGTGCTGGACCTCTGCACGTGGGGCATGTGTGGGTATGTACATGTACC |
| | | TGTGTTCTTGGTGTGTGTGTGTGTGTGTGTGTGTGTGTGTGTCTAGAGCTG |
| | | GGGTGCAACTATGGGGCCCCTCGGGACATGTCCCAGCCAATGCCTGCTTT |
| | | GACCAGAGGAGTGTCCACGTGGCTCAGGTGGTCGAGTATCTCATACCGCC |
| | | CTAGCACACGTGTGACTCCTTTCCCCTATTGTCTACACGCGTAGGAACAG |
| | | AGAAACAGGAGAATATGGGCCAAACAGGATATCTGTGGTAAGCAGTTCC |
| | | TGCCCCGGCTCAGGGCCAAGAACAGTTGGAACAGCAGAATATGGGCCAA |
| | | ACAGGATATCTGTGGTAAGCAGTTCCTGCCCCGGCTCAGGGCCAAGAACA |
| | | GATGGTCCCCAGATGCGGTCCCGCCCTCAGCAGTTTCTAGAGAACCATCA |
| | | GATGTTTCCAGGGTGCCCCAAGGACCTGAAATGACCCTGTGCCTTATTTG |
| | | AACTAACCAATCAGTTCGCTTCTCGCTTCTGTTCGCGCGCTTCTGCTCCCC |
| | | GAGCTCTATATAAGCAGAGCTCGTTTAGTGAACCGTCAGATCGCCTGGAG |
| | | ACGCCATCCACGCTGTTTTGACTTCCATAGAAGGATCTCGAGGCCACCAT |
| | | GCCTAATCCTCGGCCTGGAAAGCCTAGCGCTCCTTCTCTTGCTCTGGGACC |
| | | TTCTCCTGGCGCCTCTCCATCTTGGAGAGCCGCTCCTAAAGCCAGCGATCT |
| | | GCTGGGAGCTAGAGGACCTGGCGGCACATTTCAGGGCAGAGATCTTAGA |
| | | GGCGGAGCCCACGCTAGCTCCTCCAGCCTTAATCCTATGCCTCCTAGCCA |
| | | GCTCCAGCTGCCTACACTGCCTCTGGTTATGGTGGCTCCTAGCGGAGCTA |
| | | GACTGGGCCCTCTGCCTCATCTGCAAGCTCTGCTGCAGGACAGACCCCAC |
| | | TTCATGCACCAGCTGAGCACCGTGGATGCCCACGCAAGAACACCTGTGCT |
| | | GCAGGTTCACCCTCTGGAATCCCCAGCCATGATCAGCCTGACACCTCCAA |
| | | CAACAGCCACCGGCGTGTTCAGCCTGAAAGCCAGACCTGGACTGCCTCCT |
| | | GGCATCAATGTGGCCAGCCTGGAATGGGTGTCCAGAGAACCTGCTCTGCT |
| | | GTGCACATTCCCCAATCCAAGCGCTCCCAGAAAGGACAGCACACTGTCTG |
| | | CCGTGCCTCAGAGCAGCTATCCCCTGCTTGCTAACGGCGTGTGCAAGTGG |
| | | CCTGGATGCGAGAAGGTGTTCGAGGAACCCGAGGACTTCCTGAAGCACT |
| | | GCCAGGCCGATCATCTGCTGGACGAGAAAGGCAGAGCCCAGTGTCTGCT |
| | | CCAGCGCGAGATGGTGCAGTCTCTGGAACAGCAGCTGGTCCTGGAAAAA |
| | | GAAAAGCTGAGCGCCATGCAGGCCCACCTGGCCGGAAAAATGGCCCTGA |
| | | CAAAGGCCAGCAGCGTGGCCTCTTCTGATAAGGGCAGCTGCTGCATTGTG |
| | | GCCGCTGGATCTCAGGGACCTGTGGTTCCTGCTTGGAGCGGACCTAGAGA |
| | | GGCCCCTGATTCTCTGTTTGCCGTGCGGAGACACCTGTGGGGCTCTCACG |
| | | GCAACTCTACTTTCCCCGAGTTCCTGCACAACATGGACTACTTCAAGTTCC |
| | | ACAACATGCGGCCTCCATTCACCTACGCCACACTGATCAGATGGGCCATT |
| | | CTGGAAGCCCCTGAGAAGCAGAGAACCCTGAACGAGATCTACCACTGGT |
| | | TTACCCGGATGTTCGCCTTCTTCCGGAATCACCCTGCCACCTGGAAGAAC |
| | | GCCATCCGGCACAATCTGAGCCTGCACAAGTGCTTCGTGCGCGTGGAATC |
| | | TGAGAAAGGCGCCGTGTGGACAGTGGACGAGCTGGAATTCAGAAAGAAG |
| | | AGAAGCCAGCGGCCTAGCCGGTGCAGCAATCCTACACCTGGACCTGGAA |
| | | GCGGAGCGACTAACTTCAGCCTGCTGAAGCAGGCCGGAGATGTGGAGGA |
| | | AAACCCTGGACCGATGGTGAGCAAGGGCGAGGAGCTGTTCACCGGGGTG |
| | | GTGCCCATCCTGGTCGAGCTGGACGGCGACGTAAACGGCCACAAGTTCA |
| | | GCGTGTCTGGCGAGGGCGAGGGCGATGCCACCTACGGCAAGCTGACCCT |
| | | GAAGTTCATCTGCACCACCGGCAAGCTGCCCGTGCCCTGGCCCACCCTCG |
| | | TGACCACCCTGACCTACGGCGTGCAGTGCTTCAGCCGCTACCCCGACCAC |
| | | ATGAAGCAGCACGACTTCTTCAAGTCCGCCATGCCCGAAGGCTACGTCCA |
| | | GGAGCGCACCATCTTCTTCAAGGACGACGGCAACTACAAGACCCGCGCC |
| | | GAGGTGAAGTTCGAGGGCGACACCCTGGTGAACCGCATCGAGCTGAAGG |
| | | GCATCGACTTCAAGGAGGACGGCAACATCCTGGGGCACAAGCTGGAGTA |
| | | CAACTACAACAGCCACAACGTCTATATCATGGCCGACAAGCAGAAGAAC |
| | | GGCATCAAGGCGAACTTCAAGATCCGCCACAACATCGAGGACGGCAGCG |
| | | TGCAGCTCGCCGACCACTACCAGCAGAACACCCCCATCGGCGACGGCCCC |
| | | GTGCTGCTGCCCGACAACCACTACCTGAGCACCCAGTCCGCCCTGAGCAA |
| | | AGACCCCAACGAGAAGCGCGATCACATGGTCCTGCTGGAGTTCGTGACC |
| | | GCCGCCGGGATCACTCTCGGCATGGACGAGCTGTACAAGTAATGAAAGC |
| | | TTGATAATCAACCTCTGGATTACAAAATTTGTGAAAGATTGACTGGTATT |
| | | CTTAACTATGTTGCTCCTTTTACGCTATGTGGATACGCTGCTTTAATGCCT |
| | | TTGTATCATGCTATTGCTTCCCGTATGGCTTTCATTTTCTCCTCCTTGTATA |
| | | AATCCTGGTTAGTTCTTGCCACGGCGGAACTCATCGCCGCCTGCCTTGCCC |
| | | GCTGCTGGACAGGGGCTCGGCTGTTGGGCACTGACAATTCCGTGGGTCGA |
| | | CTGCTTTATTTGTGAAATTTGTGATGCTATTGCTTTATTTGTAACCATTATA |
| | | AGCTGCAATAAACAAGTTAACAACAACAATTGCATTCATTTTATGTTTCA |
| | | GGTTCAGGGGGAGATGTGGGAGGTTTTTTAAAGCACTAGTGTGAGGCCCT |
| | | GGGCCCAGGATGGGGCAGGCAGGGTGGGGTACCTGGACCTACAGGTGCC |
| | | GACCTTTACTGTGGCACTGGGCGGGAGGGGGGCTGGCTGGGGCACAGGA |
| | | AGTGGTTTCTGGGTCCCAGGCAAGTCTGTGACTTATGCAGATGTTGCAGG |
| | | GCCAAGAAAATCCCCACCTGCCAGGCCTCAGAGATTGGAGGCTCTCCCCG |
| | | ACCTCCCAATCCCTGTCTCAGGAGAGGAGGAGGCCGTGGATCCTACGTAG |
| | | ATAAGTAGCATGGCGGGTTAATCATTAACTACAAGGAACCCCTAGTGATG |
| | | GAGTTGGCCACTCCCTCTCTGCGCGCTCGCTCGCTCACTGAGGCCGGGCG |
| | | ACCAAAGGTCGCCCGACGCCCGGGCTTTGCCCGGGCGGCCTCAGTGAGC |
| | | GAGCGAGCGCGCCAGCTGGCGTAATAGCGAAGAGGCCCGCACCGATCGC |
| | | CCTTCCCAACAGTTGCGCAGCCTGAATGGCGAATGGCGATTCCGTTGCAA |
| | | TGGCTGGCGGTAATATTGTTCTGGATATTACCAGCAAGGCCGATAGTTTG |
| | | AGTTCTTCTACTCAGGCAAGTGATGTTATTACTAATCAAAGAAGTATTGC |
| | | GACAACGGTTAATTTGCGTGATGGACAGACTCTTTTACTCGGTGGCCTCA |
| | | CTGATTATAAAAACACTTCTCAGGATTCTGGCGTACCGTTCCTGTCTAAA |
| | | ATCCCTTTAATCGGCCTCCTGTTTAGCTCCCGCTCTGATTCTAACGAGGAA |
| | | AGCACGTTATACGTGCTCGTCAAAGCAACCATAGTACGCGCCCTGTAGCG |
| | | GCGCATTAAGCGCGGCGGGTGTGGTGGTTACGCGCAGCGTGACCGCTAC |
| | | ACTTGCCAGCGCCCTAGCGCCCGCTCCTTTCGCTTTCTTCCCTTCCTTTCTC |
| | | GCCACGTTCGCCGGCTTTCCCCGTCAAGCTCTAAATCGGGGGCTCCCTTTA |
| | | GGGTTCCGATTTAGTGCTTTACGGCACCTCGACCCCAAAAAACTTGATTA |
| | | GGGTGATGGTTCACGTAGTGGGCCATCGCCCTGATAGACGGTTTTTCGCC |
| | | CTTTGACGTTGGAGTCCACGTTCTTTAATAGTGGACTCTTGTTCCAAACTG |
| | | GAACAACACTCAACCCTATCTCGGTCTATTCTTTTGATTTATAAGGGATTT |
| | | TGCCGATTTCGGCCTATTGGTTAAAAAATGAGCTGATTTAACAAAAATTT |
| | | AACGCGAATTTTAACAAAATATTAACGTTTACAATTTAAATATTTGCTTAT |
| | | ACAATCTTCCTGTTTTTGGGGCTTTTCTGATTATCAACCGGGGTACATATG |
| | | ATTGACATGCTAGTTTTACGATTACCGTTCATCGATTCTCTTGTTTGCTCC |
| | | AGACTCTCAGGCAATGACCTGATAGCCTTTGTAGAGACCTCTCAAAAATA |
| | | GCTACCCTCTCCGGCATGAATTTATCAGCTAGAACGGTTGAATATCATAT |
| | | TGATGGTGATTTGACTGTCTCCGGCCTTTCTCACCCGTTTGAATCTTTACC |
| | | TACACATTACTCAGGCATTGCATTTAAAATATATGAGGGTTCTAAAAATT |
| | | TTTATCCTTGCGTTGAAATAAAGGCTTCTCCCGCAAAAGTATTACAGGGT |
| | | CATAATGTTTTTGGTACAACCGATTTAGCTTTATGCTCTGAGGCTTTATTG |
| | | CTTAATTTTGCTAATTCTTTGCCTTGCCTGTATGATTTATTGGATGTTGGA |
| | | ATCGCCTGATGCGGTATTTTCTCCTTACGCATCTGTGCGGTATTTCACACC |
| | | GCATATGGTGCACTCTCAGTACAATCTGCTCTGATGCCGCATAGTTAAGC |
| | | CAGCCCCGACACCCGCCAACACCCGCTGACGCGCCCTGACGGGCTTGTCT |
| | | GCTCCCGGCATCCGCTTACAGACAAGCTGTGACCGTCTCCGGGAGCTGCA |
| | | TGTGTCAGAGGTTTTCACCGTCATCACCGAAACGCGCGAGACGAAAGGG |
| | | CCTCGTGATACGCCTATTTTTATAGGTTAATGTCATGATAATAATGGTTTC |
| | | TTAGACGTCAGGTGGCACTTTTCGGGGAAATGTGCGCGGAACCCCTATTT |
| | | GTTTATTTTTCTAAATACATTCAAATATGTATCCGCTCATGAGACAATAAC |
| | | CCTGATAAATGCTTCAATAATATTGAAAAAGGAAGAGTATGAGTATTCAA |
| | | CATTTCCGTGTCGCCCTTATTCCCTTTTTTGCGGCATTTTGCCTTCCTGTTT |
| | | TTGCTCACCCAGAAACGCTGGTGAAAGTAAAAGATGCTGAAGATCAGTT |
| | | GGGTGCACGAGTGGGTTACATCGAACTGGATCTCAACAGCGGTAAGATC |
| | | CTTGAGAGTTTTCGCCCCGAAGAACGTTTTCCAATGATGAGCACTTTTAA |
| | | AGTTCTGCTATGTGGCGCGGTATTATCCCGTATTGACGCCGGGCAAGAGC |
| | | AACTCGGTCGCCGCATACACTATTCTCAGAATGACTTGGTTGAGTACTCA |
| | | CCAGTCACAGAAAAGCATCTTACGGATGGCATGACAGTAAGAGAATTAT |
| | | GCAGTGCTGCCATAACCATGAGTGATAACACTGCGGCCAACTTACTTCTG |
| | | ACAACGATCGGAGGACCGAAGGAGCTAACCGCTTTTTTGCACAACATGG |
| | | GGGATCATGTAACTCGCCTTGATCGTTGGGAACCGGAGCTGAATGAAGCC |
| | | ATACCAAACGACGAGCGTGACACCACGATGCCTGTAGCAATGGCAACAA |
| | | CGTTGCGCAAACTATTAACTGGCGAACTACTTACTCTAGCTTCCCGGCAA |
| | | CAATTAATAGACTGGATGGAGGCGGATAAAGTTGCAGGACCACTTCTGC |
| | | GCTCGGCCCTTCCGGCTGGCTGGTTTATTGCTGATAAATCTGGAGCCGGT |
| | | GAGCGTGGGTCTCGCGGTATCATTGCAGCACTGGGGCCAGATGGTAAGCC |
| | | CTCCCGTATCGTAGTTATCTACACGACGGGGAGTCAGGCAACTATGGATG |
| | | AACGAAATAGACAGATCGCTGAGATAGGTGCCTCACTGATTAAGCATTG |
| | | GTAACTGTCAGACCAAGTTTACTCATATATACTTTAGATTGATTTAAAACT |
| | | TCATTTTTAATTTAAAAGGATCTAGGTGAAGATCCTTTTTGATAATCTCAT |
| | | GACCAAAATCCCTTAACGTGAGTTTTCGTTCCACTGAGCGTCAGACCCC |
| 143 | <#3020 | GTAGAAAAGATCAAAGGATCTTCTTGAGATCCTTTTTTTCTGCGCGTAATC |
| | pAAV_FOXP3. | TGCTGCTTGCAAACAAAAAAACCACCGCTACCAGCGGTGGTTTGTTTGCC |
| | 045_MND.FOX | GGATCAAGAGCTACCAACTCTTTTTCCGAAGGTAACTGGCTTCAGCAGAG |
| | P3geneartCDS.P | CGCAGATACCAAATACTGTCCTTCTAGTGTAGCCGTAGTTAGGCCACCAC |
| | 2A.LNGFR.WP | TTCAAGAACTCTGTAGCACCGCCTACATACCTCGCTCTGCTAATCCTGTTA |
| | RE3.pA_06> | CCAGTGGCTGCTGCCAGTGGCGATAAGTCGTGTCTTACCGGGTTGGACTC |
| | | AAGACGATAGTTACCGGATAAGGCGCAGCGGTCGGGCTGAACGGGGGGT |
| | | TCGTGCACACAGCCCAGCTTGGAGCGAACGACCTACACCGAACTGAGAT |
| | | ACCTACAGCGTGAGCTATGAGAAAGCGCCACGCTTCCCGAAGGGAGAAA |
| | | GGCGGACAGGTATCCGGTAAGCGGCAGGGTCGGAACAGGAGAGCGCACG |
| | | AGGGAGCTTCCAGGGGGAAACGCCTGGTATCTTTATAGTCCTGTCGGGTT |
| | | TCGCCACCTCTGACTTGAGCGTCGATTTTTGTGATGCTCGTCAGGGGGGC |
| | | GGAGCCTATGGAAAAACGCCAGCAACGCGGCCTTTTTACGGTTCCTGGCC |
| | | TTTTGCTGGCCTTTTGCTCACATGTTCTTTCCTGCGTTATCCCCTGATTCTG |
| | | TGGATAACCGTATTACCGCCTTTGAGTGAGCTGATACCGCTCGCCGCAGC |
| | | CGAACGACCGAGCGCAGCGAGTCAGTGAGCGAGGAAGCGGAAGAGCGC |
| | | CCAATACGCAAACCGCCTCTCCCCGCGCGTTGGCCGATTCATTAATGCAG |
| | | CTGCGCGCTCGCTCGCTCACTGAGGCCGCCCGGGCAAAGCCCGGGCGTCG |
| | | GGCGACCTTTGGTCGCCCGGCCTCAGTGAGCGAGCGAGCGCGCAGAGAG |
| | | GGAGTGGCCAACTCCATCACTAGGGGTTCCTTGTAGTTAATGATTAACCC |
| | | GCCATGCTACTTATCTACGTAGCGGCCGCAGTCCATGCCTAGTCACTGGG |
| | | GCAAAATAGGACTCCGAGGAGAAAGTCCGAGACCAGCTCCGGCAAGATG |
| | | AGCAAACACAGCCTGTGCAGGGTGCAGGGAGGGCTAGAGGCCTGAGGCT |
| | | TGAAACAGCTCTCAAGTGGAGGGGGAAACAACCATTGCCCTCATAGAGG |
| | | ACACATCCACACCAGGGCTGTGCTAGCGTGGGCAGGCAAGCCAGGTGCT |
| | | GGACCTCTGCACGTGGGGCATGTGTGGGTATGTACATGTACCTGTGTTCT |
| | | TGGTGTGTGTGTGTGTGTGTGTGTGTGTGTGTGTCTAGAGCTGGGGTGCA |
| | | ACTATGGGGCCCCTCGGGACATGTCCCAGCCAATGCCTGCTTTGACCAGA |
| | | GGAGTGTCCACGTGGCTCAGGTGGTCGAGTATCTCATACCGCCCTAGCAC |
| | | ACGTGTGACTCCTTTCCCCTATTGTCTACACGCGTAGGAACAGAGAAACA |
| | | GGAGAATATGGGCCAAACAGGATATCTGTGGTAAGCAGTTCCTGCCCCG |
| | | GCTCAGGGCCAAGAACAGTTGGAACAGCAGAATATGGGCCAAACAGGAT |
| | | ATCTGTGGTAAGCAGTTCCTGCCCCGGCTCAGGGCCAAGAACAGATGGTC |
| | | CCCAGATGCGGTCCCGCCCTCAGCAGTTTCTAGAGAACCATCAGATGTTT |
| | | CCAGGGTGCCCCAAGGACCTGAAATGACCCTGTGCCTTATTTGAACTAAC |
| | | CAATCAGTTCGCTTCTCGCTTCTGTTCGCGCGCTTCTGCTCCCCGAGCTCT |
| | | ATATAAGCAGAGCTCGTTTAGTGAACCGTCAGATCGCCTGGAGACGCCAT |
| | | CCACGCTGTTTTGACTTCCATAGAAGGATCTCGAGGCCACCATGCCTAAT |
| | | CCTCGGCCTGGAAAGCCTAGCGCTCCTTCTCTTGCTCTGGGACCTTCTCCT |
| | | GGCGCCTCTCCATCTTGGAGAGCCGCTCCTAAAGCCAGCGATCTGCTGGG |
| | | AGCTAGAGGACCTGGCGGCACATTTCAGGGCAGAGATCTTAGAGGCGGA |
| | | GCCCACGCTAGCTCCTCCAGCCTTAATCCTATGCCTCCTAGCCAGCTCCAG |
| | | CTGCCTACACTGCCTCTGGTTATGGTGGCTCCTAGCGGAGCTAGACTGGG |
| | | CCCTCTGCCTCATCTGCAAGCTCTGCTGCAGGACAGACCCCACTTCATGC |
| | | ACCAGCTGAGCACCGTGGATGCCCACGCAAGAACACCTGTGCTGCAGGTT |
| | | CACCCTCTGGAATCCCCAGCCATGATCAGCCTGACACCTCCAACAACAGC |
| | | CACCGGCGTGTTCAGCCTGAAAGCCAGACCTGGACTGCCTCCTGGCATCA |
| | | ATGTGGCCAGCCTGGAATGGGTGTCCAGAGAACCTGCTCTGCTGTGCACA |
| | | TTCCCCAATCCAAGCGCTCCCAGAAAGGACAGCACACTGTCTGCCGTGCC |
| | | TCAGAGCAGCTATCCCCTGCTTGCTAACGGCGTGTGCAAGTGGCCTGGAT |
| | | GCGAGAAGGTGTTCGAGGAACCCGAGGACTTCCTGAAGCACTGCCAGGC |
| | | CGATCATCTGCTGGACGAGAAAGGCAGAGCCCAGTGTCTGCTCCAGCGC |
| | | GAGATGGTGCAGTCTCTGGAACAGCAGCTGGTCCTGGAAAAAGAAAAGC |
| | | TGAGCGCCATGCAGGCCCACCTGGCCGGAAAAATGGCCCTGACAAAGGC |
| | | CAGCAGCGTGGCCTCTTCTGATAAGGGCAGCTGCTGCATTGTGGCCGCTG |
| | | GATCTCAGGGACCTGTGGTTCCTGCTTGGAGCGGACCTAGAGAGGCCCCT |
| | | GATTCTCTGTTTGCCGTGCGGAGACACCTGTGGGGCTCTCACGGCAACTC |
| | | TACTTTCCCCGAGTTCCTGCACAACATGGACTACTTCAAGTTCCACAACAT |
| | | GCGGCCTCCATTCACCTACGCCACACTGATCAGATGGGCCATTCTGGAAG |
| | | CCCCTGAGAAGCAGAGAACCCTGAACGAGATCTACCACTGGTTTACCCGG |
| | | ATGTTCGCCTTCTTCCGGAATCACCCTGCCACCTGGAAGAACGCCATCCG |
| | | GCACAATCTGAGCCTGCACAAGTGCTTCGTGCGCGTGGAATCTGAGAAAG |
| | | GCGCCGTGTGGACAGTGGACGAGCTGGAATTCAGAAAGAAGAGAAGCCA |
| | | GCGGCCTAGCCGGTGCAGCAATCCTACACCTGGACCTGGAAGCGGAGCG |
| | | ACTAACTTCAGCCTGCTGAAGCAGGCCGGAGATGTGGAGGAAAACCCTG |
| | | GACCGATGGGGGCAGGTGCCACCGGACGAGCCATGGACGGGCCGCGCCT |
| | | GCTGCTGTTGCTGCTTCTGGGGGTGTCCCTTGGAGGTGCCAAGGAGGCAT |
| | | GCCCCACAGGCCTGTACACACACAGCGGTGAGTGCTGCAAAGCCTGCAA |
| | | CCTGGGCGAGGGTGTGGCCCAGCCTTGTGGAGCCAACCAGACCGTGTGTG |
| | | AGCCCTGCCTGGACAGCGTGACGTTCTCCGACGTGGTGAGCGCGACCGAG |
| | | CCGTGCAAGCCGTGCACCGAGTGCGTGGGGCTCCAGAGCATGTCGGCGC |
| | | CGTGCGTGGAGGCCGACGACGCCGTGTGCCGCTGCGCCTACGGCTACTAC |
| | | CAGGATGAGACGACTGGGCGCTGCGAGGCGTGCCGCGTGTGCGAGGCGG |
| | | GCTCGGGCCTCGTGTTCTCCTGCCAGGACAAGCAGAACACCGTGTGCGAG |
| | | GAGTGCCCCGACGGCACGTATTCCGACGAGGCCAACCACGTGGACCCGT |
| | | GCCTGCCCTGCACCGTGTGCGAGGACACCGAGCGCCAGCTCCGCGAGTGC |
| | | ACACGCTGGGCCGACGCCGAGTGCGAGGAGATCCCTGGCCGTTGGATTA |
| | | CACGGTCCACACCCCCAGAGGGCTCGGACAGCACAGCCCCCAGCACCCA |
| | | GGAGCCTGAGGCACCTCCAGAACAAGACCTCATAGCCAGCACGGTGGCA |
| | | GGTGTGGTGACCACAGTGATGGGCAGCTCCCAGCCCGTGGTGACCCGAG |
| | | GCACCACCGACAACCTCATCCCTGTCTATTGCTCCATCCTGGCTGCTGTGG |
| | | TTGTGGGTCTTGTGGCCTACATAGCCTTCAAGAGGTGAAAGCTTCCACGG |
| | | AATTGTCAGTGCCCAACAGCCGAGCCCCTGTCCAGCAGCGGGCAAGGCA |
| | | GGCGGCGATGAGTTCCGCCGTGGCAAGAACTAACCAGGATTTATACAAG |
| | | GAGGAGAAAATGAAAGCCATACGGGAAGCAATAGCATGATACAAAGGC |
| | | ATTAAAGCAGCGTATCCACATAGCGTAAAAGGAGCAACATAGTTAAGAA |
| | | TACCAGTCAATCTTTCACAAATTTTGTAATCCAGAGGTTGATTATCGTCGA |
| | | CTGCTTTATTTGTGAAATTTGTGATGCTATTGCTTTATTTGTAACCATTATA |
| | | AGCTGCAATAAACAAGTTAACAACAACAATTGCATTCATTTTATGTTTCA |
| | | GGTTCAGGGGGAGATGTGGGAGGTTTTTTAAAGCACTAGTGTGAGGCCCT |
| | | GGGCCCAGGATGGGGCAGGCAGGGTGGGGTACCTGGACCTACAGGTGCC |
| | | GACCTTTACTGTGGCACTGGGCGGGAGGGGGGCTGGCTGGGGCACAGGA |
| | | AGTGGTTTCTGGGTCCCAGGCAAGTCTGTGACTTATGCAGATGTTGCAGG |
| | | GCCAAGAAAATCCCCACCTGCCAGGCCTCAGAGATTGGAGGCTCTCCCCG |
| | | ACCTCCCAATCCCTGTCTCAGGAGAGGAGGAGGCCGTATTGTAGTCCCAT |
| | | GAGCATAGCTATGTGTCCCCATCCCCATGTGACAAGAGAAGAGGACTGG |
| | | GGCCAAGTAGGTGAGGTGACAGGGCTGAGGCCAGCTCTGCAACTTATTA |
| | | GCTGTTTGATCTTTAAAAAGTTACTCGATCTCCATGAGCCTCAGTTTCCAT |
| | | ACGTGTAAAAGGGGGATGATCATAGCATCTACCATGTGGGCTTGCAGTGC |
| | | AGAGTATTTGAATTAGACACAGAACAGTGAGGATCAGGATGGCCTCTCA |
| | | CCCACCTGCCTTTCTGCCCAGCTGCCCACACTGCCCCTAGTCATGGTGGCA |
| | | CCCTCCGGGGCACGGCTGGGCCCCTTGCCCCACTTACAGGCACCGCGGCG |
| | | CTACGTAGATAAGTAGCATGGCGGGTTAATCATTAACTACAAGGAACCCC |
| | | TAGTGATGGAGTTGGCCACTCCCTCTCTGCGCGCTCGCTCGCTCACTGAG |
| | | GCCGGGCGACCAAAGGTCGCCCGACGCCCGGGCTTTGCCCGGGCGGCCT |
| | | CAGTGAGCGAGCGAGCGCGCCAGCTGGCGTAATAGCGAAGAGGCCCGCA |
| | | CCGATCGCCCTTCCCAACAGTTGCGCAGCCTGAATGGCGAATGGCGATTC |
| | | CGTTGCAATGGCTGGCGGTAATATTGTTCTGGATATTACCAGCAAGGCCG |
| | | ATAGTTTGAGTTCTTCTACTCAGGCAAGTGATGTTATTACTAATCAAAGA |
| | | AGTATTGCGACAACGGTTAATTTGCGTGATGGACAGACTCTTTTACTCGG |
| | | TGGCCTCACTGATTATAAAAACACTTCTCAGGATTCTGGCGTACCGTTCCT |
| | | GTCTAAAATCCCTTTAATCGGCCTCCTGTTTAGCTCCCGCTCTGATTCTAA |
| | | CGAGGAAAGCACGTTATACGTGCTCGTCAAAGCAACCATAGTACGCGCC |
| | | CTGTAGCGGCGCATTAAGCGCGGCGGGTGTGGTGGTTACGCGCAGCGTG |
| | | ACCGCTACACTTGCCAGCGCCCTAGCGCCCGCTCCTTTCGCTTTCTTCCCT |
| | | TCCTTTCTCGCCACGTTCGCCGGCTTTCCCCGTCAAGCTCTAAATCGGGGG |
| | | CTCCCTTTAGGGTTCCGATTTAGTGCTTTACGGCACCTCGACCCCAAAAA |
| | | ACTTGATTAGGGTGATGGTTCACGTAGTGGGCCATCGCCCTGATAGACGG |
| | | TTTTTCGCCCTTTGACGTTGGAGTCCACGTTCTTTAATAGTGGACTCTTGT |
| | | TCCAAACTGGAACAACACTCAACCCTATCTCGGTCTATTCTTTTGATTTAT |
| | | AAGGGATTTTGCCGATTTCGGCCTATTGGTTAAAAAATGAGCTGATTTAA |
| | | CAAAAATTTAACGCGAATTTTAACAAAATATTAACGTTTACAATTTAAAT |
| | | ATTTGCTTATACAATCTTCCTGTTTTTGGGGCTTTTCTGATTATCAACCGG |
| | | GGTACATATGATTGACATGCTAGTTTTACGATTACCGTTCATCGATTCTCT |
| | | TGTTTGCTCCAGACTCTCAGGCAATGACCTGATAGCCTTTGTAGAGACCT |
| | | CTCAAAAATAGCTACCCTCTCCGGCATGAATTTATCAGCTAGAACGGTTG |
| | | AATATCATATTGATGGTGATTTGACTGTCTCCGGCCTTTCTCACCCGTTTG |
| | | AATCTTTACCTACACATTACTCAGGCATTGCATTTAAAATATATGAGGGTT |
| | | CTAAAAATTTTTATCCTTGCGTTGAAATAAAGGCTTCTCCCGCAAAAGTA |
| | | TTACAGGGTCATAATGTTTTTGGTACAACCGATTTAGCTTTATGCTCTGAG |
| | | GCTTTATTGCTTAATTTTGCTAATTCTTTGCCTTGCCTGTATGATTTATTGG |
| | | ATGTTGGAATCGCCTGATGCGGTATTTTCTCCTTACGCATCTGTGCGGTAT |
| | | TTCACACCGCATATGGTGCACTCTCAGTACAATCTGCTCTGATGCCGCAT |
| | | AGTTAAGCCAGCCCCGACACCCGCCAACACCCGCTGACGCGCCCTGACG |
| | | GGCTTGTCTGCTCCCGGCATCCGCTTACAGACAAGCTGTGACCGTCTCCG |
| | | GGAGCTGCATGTGTCAGAGGTTTTCACCGTCATCACCGAAACGCGCGAGA |
| | | CGAAAGGGCCTCGTGATACGCCTATTTTTATAGGTTAATGTCATGATAAT |
| | | AATGGTTTCTTAGACGTCAGGTGGCACTTTTCGGGGAAATGTGCGCGGAA |
| | | CCCCTATTTGTTTATTTTTCTAAATACATTCAAATATGTATCCGCTCATGA |
| | | GACAATAACCCTGATAAATGCTTCAATAATATTGAAAAAGGAAGAGTAT |
| | | GAGTATTCAACATTTCCGTGTCGCCCTTATTCCCTTTTTTGCGGCATTTTGC |
| | | CTTCCTGTTTTTGCTCACCCAGAAACGCTGGTGAAAGTAAAAGATGCTGA |
| | | AGATCAGTTGGGTGCACGAGTGGGTTACATCGAACTGGATCTCAACAGCG |
| | | GTAAGATCCTTGAGAGTTTTCGCCCCGAAGAACGTTTTCCAATGATGAGC |
| | | ACTTTTAAAGTTCTGCTATGTGGCGCGGTATTATCCCGTATTGACGCCGGG |
| | | CAAGAGCAACTCGGTCGCCGCATACACTATTCTCAGAATGACTTGGTTGA |
| | | GTACTCACCAGTCACAGAAAAGCATCTTACGGATGGCATGACAGTAAGA |
| | | GAATTATGCAGTGCTGCCATAACCATGAGTGATAACACTGCGGCCAACTT |
| | | ACTTCTGACAACGATCGGAGGACCGAAGGAGCTAACCGCTTTTTTGCACA |
| | | ACATGGGGGATCATGTAACTCGCCTTGATCGTTGGGAACCGGAGCTGAAT |
| | | GAAGCCATACCAAACGACGAGCGTGACACCACGATGCCTGTAGCAATGG |
| | | CAACAACGTTGCGCAAACTATTAACTGGCGAACTACTTACTCTAGCTTCC |
| | | CGGCAACAATTAATAGACTGGATGGAGGCGGATAAAGTTGCAGGACCAC |
| | | TTCTGCGCTCGGCCCTTCCGGCTGGCTGGTTTATTGCTGATAAATCTGGAG |
| | | CCGGTGAGCGTGGGTCTCGCGGTATCATTGCAGCACTGGGGCCAGATGGT |
| | | AAGCCCTCCCGTATCGTAGTTATCTACACGACGGGGAGTCAGGCAACTAT |
| | | GGATGAACGAAATAGACAGATCGCTGAGATAGGTGCCTCACTGATTAAG |
| | | CATTGGTAACTGTCAGACCAAGTTTACTCATATATACTTTAGATTGATTTA |
| | | AAACTTCATTTTTAATTTAAAAGGATCTAGGTGAAGATCCTTTTTGATAAT |
| | | CTCATGACCAAAATCCCTTAACGTGAGTTTTCGTTCCACTGAGCGTCAGA |
| | | CCCC |
| 144 | <#3021 | GTAGAAAAGATCAAAGGATCTTCTTGAGATCCTTTTTTTCTGCGCGTAATC |
| | pAAV_FOXP3. | TGCTGCTTGCAAACAAAAAAACCACCGCTACCAGCGGTGGTTTGTTTGCC |
| | 025_MND.FOX | GGATCAAGAGCTACCAACTCTTTTTCCGAAGGTAACTGGCTTCAGCAGAG |
| | P3geneartCDS.P | CGCAGATACCAAATACTGTCCTTCTAGTGTAGCCGTAGTTAGGCCACCAC |
| | 2A.LNGFR.WP | TTCAAGAACTCTGTAGCACCGCCTACATACCTCGCTCTGCTAATCCTGTTA |
| | RE3.pA_025> | CCAGTGGCTGCTGCCAGTGGCGATAAGTCGTGTCTTACCGGGTTGGACTC |
| | | AAGACGATAGTTACCGGATAAGGCGCAGCGGTCGGGCTGAACGGGGGGT |
| | | TCGTGCACACAGCCCAGCTTGGAGCGAACGACCTACACCGAACTGAGAT |
| | | ACCTACAGCGTGAGCTATGAGAAAGCGCCACGCTTCCCGAAGGGAGAAA |
| | | GGCGGACAGGTATCCGGTAAGCGGCAGGGTCGGAACAGGAGAGCGCACG |
| | | AGGGAGCTTCCAGGGGGAAACGCCTGGTATCTTTATAGTCCTGTCGGGTT |
| | | TCGCCACCTCTGACTTGAGCGTCGATTTTTGTGATGCTCGTCAGGGGGGC |
| | | GGAGCCTATGGAAAAACGCCAGCAACGCGGCCTTTTTACGGTTCCTGGCC |
| | | TTTTGCTGGCCTTTTGCTCACATGTTCTTTCCTGCGTTATCCCCTGATTCTG |
| | | TGGATAACCGTATTACCGCCTTTGAGTGAGCTGATACCGCTCGCCGCAGC |
| | | CGAACGACCGAGCGCAGCGAGTCAGTGAGCGAGGAAGCGGAAGAGCGC |
| | | CCAATACGCAAACCGCCTCTCCCCGCGCGTTGGCCGATTCATTAATGCAG |
| | | CTGCGCGCTCGCTCGCTCACTGAGGCCGCCCGGGCAAAGCCCGGGCGTCG |
| | | GGCGACCTTTGGTCGCCCGGCCTCAGTGAGCGAGCGAGCGCGCAGAGAG |
| | | GGAGTGGCCAACTCCATCACTAGGGGTTCCTTGTAGTTAATGATTAACCC |
| | | GCCATGCTACTTATCTACGTAGCGGCCGCTGCTAGCGTGGGCAGGCAAGC |
| | | CAGGTGCTGGACCTCTGCACGTGGGGCATGTGTGGGTATGTACATGTACC |
| | | TGTGTTCTTGGTGTGTGTGTGTGTGTGTGTGTGTGTGTGTGTCTAGAGCTG |
| | | GGGTGCAACTATGGGGCCCCTCGGGACATGTCCCAGCCAATGCCTGCTTT |
| | | GACCAGAGGAGTGTCCACGTGGCTCAGGTGGTCGAGTATCTCATACCGCC |
| | | CTAGCACACGTGTGACTCCTTTCCCCTATTGTCTACACGCGTAGGAACAG |
| | | AGAAACAGGAGAATATGGGCCAAACAGGATATCTGTGGTAAGCAGTTCC |
| | | TGCCCCGGCTCAGGGCCAAGAACAGTTGGAACAGCAGAATATGGGCCAA |
| | | ACAGGATATCTGTGGTAAGCAGTTCCTGCCCCGGCTCAGGGCCAAGAACA |
| | | GATGGTCCCCAGATGCGGTCCCGCCCTCAGCAGTTTCTAGAGAACCATCA |
| | | GATGTTTCCAGGGTGCCCCAAGGACCTGAAATGACCCTGTGCCTTATTTG |
| | | AACTAACCAATCAGTTCGCTTCTCGCTTCTGTTCGCGCGCTTCTGCTCCCC |
| | | GAGCTCTATATAAGCAGAGCTCGTTTAGTGAACCGTCAGATCGCCTGGAG |
| | | ACGCCATCCACGCTGTTTTGACTTCCATAGAAGGATCTCGAGGCCACCAT |
| | | GCCTAATCCTCGGCCTGGAAAGCCTAGCGCTCCTTCTCTTGCTCTGGGACC |
| | | TTCTCCTGGCGCCTCTCCATCTTGGAGAGCCGCTCCTAAAGCCAGCGATCT |
| | | GCTGGGAGCTAGAGGACCTGGCGGCACATTTCAGGGCAGAGATCTTAGA |
| | | GGCGGAGCCCACGCTAGCTCCTCCAGCCTTAATCCTATGCCTCCTAGCCA |
| | | GCTCCAGCTGCCTACACTGCCTCTGGTTATGGTGGCTCCTAGCGGAGCTA |
| | | GACTGGGCCCTCTGCCTCATCTGCAAGCTCTGCTGCAGGACAGACCCCAC |
| | | TTCATGCACCAGCTGAGCACCGTGGATGCCCACGCAAGAACACCTGTGCT |
| | | GCAGGTTCACCCTCTGGAATCCCCAGCCATGATCAGCCTGACACCTCCAA |
| | | CAACAGCCACCGGCGTGTTCAGCCTGAAAGCCAGACCTGGACTGCCTCCT |
| | | GGCATCAATGTGGCCAGCCTGGAATGGGTGTCCAGAGAACCTGCTCTGCT |
| | | GTGCACATTCCCCAATCCAAGCGCTCCCAGAAAGGACAGCACACTGTCTG |
| | | CCGTGCCTCAGAGCAGCTATCCCCTGCTTGCTAACGGCGTGTGCAAGTGG |
| | | CCTGGATGCGAGAAGGTGTTCGAGGAACCCGAGGACTTCCTGAAGCACT |
| | | GCCAGGCCGATCATCTGCTGGACGAGAAAGGCAGAGCCCAGTGTCTGCT |
| | | CCAGCGCGAGATGGTGCAGTCTCTGGAACAGCAGCTGGTCCTGGAAAAA |
| | | GAAAAGCTGAGCGCCATGCAGGCCCACCTGGCCGGAAAAATGGCCCTGA |
| | | CAAAGGCCAGCAGCGTGGCCTCTTCTGATAAGGGCAGCTGCTGCATTGTG |
| | | GCCGCTGGATCTCAGGGACCTGTGGTTCCTGCTTGGAGCGGACCTAGAGA |
| | | GGCCCCTGATTCTCTGTTTGCCGTGCGGAGACACCTGTGGGGCTCTCACG |
| | | GCAACTCTACTTTCCCCGAGTTCCTGCACAACATGGACTACTTCAAGTTCC |
| | | ACAACATGCGGCCTCCATTCACCTACGCCACACTGATCAGATGGGCCATT |
| | | CTGGAAGCCCCTGAGAAGCAGAGAACCCTGAACGAGATCTACCACTGGT |
| | | TTACCCGGATGTTCGCCTTCTTCCGGAATCACCCTGCCACCTGGAAGAAC |
| | | GCCATCCGGCACAATCTGAGCCTGCACAAGTGCTTCGTGCGCGTGGAATC |
| | | TGAGAAAGGCGCCGTGTGGACAGTGGACGAGCTGGAATTCAGAAAGAAG |
| | | AGAAGCCAGCGGCCTAGCCGGTGCAGCAATCCTACACCTGGACCTGGAA |
| | | GCGGAGCGACTAACTTCAGCCTGCTGAAGCAGGCCGGAGATGTGGAGGA |
| | | AAACCCTGGACCGATGGGGGCAGGTGCCACCGGACGAGCCATGGACGGG |
| | | CCGCGCCTGCTGCTGTTGCTGCTTCTGGGGGTGTCCCTTGGAGGTGCCAA |
| | | GGAGGCATGCCCCACAGGCCTGTACACACACAGCGGTGAGTGCTGCAAA |
| | | GCCTGCAACCTGGGCGAGGGTGTGGCCCAGCCTTGTGGAGCCAACCAGA |
| | | CCGTGTGTGAGCCCTGCCTGGACAGCGTGACGTTCTCCGACGTGGTGAGC |
| | | GCGACCGAGCCGTGCAAGCCGTGCACCGAGTGCGTGGGGCTCCAGAGCA |
| | | TGTCGGCGCCGTGCGTGGAGGCCGACGACGCCGTGTGCCGCTGCGCCTAC |
| | | GGCTACTACCAGGATGAGACGACTGGGCGCTGCGAGGCGTGCCGCGTGT |
| | | GCGAGGCGGGCTCGGGCCTCGTGTTCTCCTGCCAGGACAAGCAGAACAC |
| | | CGTGTGCGAGGAGTGCCCCGACGGCACGTATTCCGACGAGGCCAACCAC |
| | | GTGGACCCGTGCCTGCCCTGCACCGTGTGCGAGGACACCGAGCGCCAGCT |
| | | CCGCGAGTGCACACGCTGGGCCGACGCCGAGTGCGAGGAGATCCCTGGC |
| | | CGTTGGATTACACGGTCCACACCCCCAGAGGGCTCGGACAGCACAGCCCC |
| | | CAGCACCCAGGAGCCTGAGGCACCTCCAGAACAAGACCTCATAGCCAGC |
| | | ACGGTGGCAGGTGTGGTGACCACAGTGATGGGCAGCTCCCAGCCCGTGG |
| | | TGACCCGAGGCACCACCGACAACCTCATCCCTGTCTATTGCTCCATCCTG |
| | | GCTGCTGTGGTTGTGGGTCTTGTGGCCTACATAGCCTTCAAGAGGTGAAA |
| | | GCTTCCACGGAATTGTCAGTGCCCAACAGCCGAGCCCCTGTCCAGCAGCG |
| | | GGCAAGGCAGGCGGCGATGAGTTCCGCCGTGGCAAGAACTAACCAGGAT |
| | | TTATACAAGGAGGAGAAAATGAAAGCCATACGGGAAGCAATAGCATGAT |
| | | ACAAAGGCATTAAAGCAGCGTATCCACATAGCGTAAAAGGAGCAACATA |
| | | GTTAAGAATACCAGTCAATCTTTCACAAATTTTGTAATCCAGAGGTTGAT |
| | | TATCGTCGACTGCTTTATTTGTGAAATTTGTGATGCTATTGCTTTATTTGTA |
| | | ACCATTATAAGCTGCAATAAACAAGTTAACAACAACAATTGCATTCATTT |
| | | TATGTTTCAGGTTCAGGGGGAGATGTGGGAGGTTTTTTAAAGCACTAGTG |
| | | TGAGGCCCTGGGCCCAGGATGGGGCAGGCAGGGTGGGGTACCTGGACCT |
| | | ACAGGTGCCGACCTTTACTGTGGCACTGGGCGGGAGGGGGGCTGGCTGG |
| | | GGCACAGGAAGTGGTTTCTGGGTCCCAGGCAAGTCTGTGACTTATGCAGA |
| | | TGTTGCAGGGCCAAGAAAATCCCCACCTGCCAGGCCTCAGAGATTGGAG |
| | | GCTCTCCCCGACCTCCCAATCCCTGTCTCAGGAGAGGAGGAGGCCGTGGA |
| | | TCCTACGTAGATAAGTAGCATGGCGGGTTAATCATTAACTACAAGGAACC |
| | | CCTAGTGATGGAGTTGGCCACTCCCTCTCTGCGCGCTCGCTCGCTCACTGA |
| | | GGCCGGGCGACCAAAGGTCGCCCGACGCCCGGGCTTTGCCCGGGCGGCC |
| | | TCAGTGAGCGAGCGAGCGCGCCAGCTGGCGTAATAGCGAAGAGGCCCGC |
| | | ACCGATCGCCCTTCCCAACAGTTGCGCAGCCTGAATGGCGAATGGCGATT |
| | | CCGTTGCAATGGCTGGCGGTAATATTGTTCTGGATATTACCAGCAAGGCC |
| | | GATAGTTTGAGTTCTTCTACTCAGGCAAGTGATGTTATTACTAATCAAAG |
| | | AAGTATTGCGACAACGGTTAATTTGCGTGATGGACAGACTCTTTTACTCG |
| | | GTGGCCTCACTGATTATAAAAACACTTCTCAGGATTCTGGCGTACCGTTC |
| | | CTGTCTAAAATCCCTTTAATCGGCCTCCTGTTTAGCTCCCGCTCTGATTCT |
| | | AACGAGGAAAGCACGTTATACGTGCTCGTCAAAGCAACCATAGTACGCG |
| | | CCCTGTAGCGGCGCATTAAGCGCGGCGGGTGTGGTGGTTACGCGCAGCGT |
| | | GACCGCTACACTTGCCAGCGCCCTAGCGCCCGCTCCTTTCGCTTTCTTCCC |
| | | TTCCTTTCTCGCCACGTTCGCCGGCTTTCCCCGTCAAGCTCTAAATCGGGG |
| | | GCTCCCTTTAGGGTTCCGATTTAGTGCTTTACGGCACCTCGACCCCAAAA |
| | | AACTTGATTAGGGTGATGGTTCACGTAGTGGGCCATCGCCCTGATAGACG |
| | | GTTTTTCGCCCTTTGACGTTGGAGTCCACGTTCTTTAATAGTGGACTCTTG |
| | | TTCCAAACTGGAACAACACTCAACCCTATCTCGGTCTATTCTTTTGATTTA |
| | | TAAGGGATTTTGCCGATTTCGGCCTATTGGTTAAAAAATGAGCTGATTTA |
| | | ACAAAAATTTAACGCGAATTTTAACAAAATATTAACGTTTACAATTTAAA |
| | | TATTTGCTTATACAATCTTCCTGTTTTTGGGGCTTTTCTGATTATCAACCGG |
| | | GGTACATATGATTGACATGCTAGTTTTACGATTACCGTTCATCGATTCTCT |
| | | TGTTTGCTCCAGACTCTCAGGCAATGACCTGATAGCCTTTGTAGAGACCT |
| | | CTCAAAAATAGCTACCCTCTCCGGCATGAATTTATCAGCTAGAACGGTTG |
| | | AATATCATATTGATGGTGATTTGACTGTCTCCGGCCTTTCTCACCCGTTTG |
| | | AATCTTTACCTACACATTACTCAGGCATTGCATTTAAAATATATGAGGGTT |
| | | CTAAAAATTTTTATCCTTGCGTTGAAATAAAGGCTTCTCCCGCAAAAGTA |
| | | TTACAGGGTCATAATGTTTTTGGTACAACCGATTTAGCTTTATGCTCTGAG |
| | | GCTTTATTGCTTAATTTTGCTAATTCTTTGCCTTGCCTGTATGATTTATTGG |
| | | ATGTTGGAATCGCCTGATGCGGTATTTTCTCCTTACGCATCTGTGCGGTAT |
| | | TTCACACCGCATATGGTGCACTCTCAGTACAATCTGCTCTGATGCCGCAT |
| | | AGTTAAGCCAGCCCCGACACCCGCCAACACCCGCTGACGCGCCCTGACG |
| | | GGCTTGTCTGCTCCCGGCATCCGCTTACAGACAAGCTGTGACCGTCTCCG |
| | | GGAGCTGCATGTGTCAGAGGTTTTCACCGTCATCACCGAAACGCGCGAGA |
| | | CGAAAGGGCCTCGTGATACGCCTATTTTTATAGGTTAATGTCATGATAAT |
| | | AATGGTTTCTTAGACGTCAGGTGGCACTTTTCGGGGAAATGTGCGCGGAA |
| | | CCCCTATTTGTTTATTTTTCTAAATACATTCAAATATGTATCCGCTCATGA |
| | | GACAATAACCCTGATAAATGCTTCAATAATATTGAAAAAGGAAGAGTAT |
| | | GAGTATTCAACATTTCCGTGTCGCCCTTATTCCCTTTTTTGCGGCATTTTGC |
| | | CTTCCTGTTTTTGCTCACCCAGAAACGCTGGTGAAAGTAAAAGATGCTGA |
| | | AGATCAGTTGGGTGCACGAGTGGGTTACATCGAACTGGATCTCAACAGCG |
| | | GTAAGATCCTTGAGAGTTTTCGCCCCGAAGAACGTTTTCCAATGATGAGC |
| | | ACTTTTAAAGTTCTGCTATGTGGCGCGGTATTATCCCGTATTGACGCCGGG |
| | | CAAGAGCAACTCGGTCGCCGCATACACTATTCTCAGAATGACTTGGTTGA |
| | | GTACTCACCAGTCACAGAAAAGCATCTTACGGATGGCATGACAGTAAGA |
| | | GAATTATGCAGTGCTGCCATAACCATGAGTGATAACACTGCGGCCAACTT |
| | | ACTTCTGACAACGATCGGAGGACCGAAGGAGCTAACCGCTTTTTTGCACA |
| | | ACATGGGGGATCATGTAACTCGCCTTGATCGTTGGGAACCGGAGCTGAAT |
| | | GAAGCCATACCAAACGACGAGCGTGACACCACGATGCCTGTAGCAATGG |
| | | CAACAACGTTGCGCAAACTATTAACTGGCGAACTACTTACTCTAGCTTCC |
| | | CGGCAACAATTAATAGACTGGATGGAGGCGGATAAAGTTGCAGGACCAC |
| | | TTCTGCGCTCGGCCCTTCCGGCTGGCTGGTTTATTGCTGATAAATCTGGAG |
| | | CCGGTGAGCGTGGGTCTCGCGGTATCATTGCAGCACTGGGGCCAGATGGT |
| | | AAGCCCTCCCGTATCGTAGTTATCTACACGACGGGGAGTCAGGCAACTAT |
| | | GGATGAACGAAATAGACAGATCGCTGAGATAGGTGCCTCACTGATTAAG |
| | | CATTGGTAACTGTCAGACCAAGTTTACTCATATATACTTTAGATTGATTTA |
| | | AAACTTCATTTTTAATTTAAAAGGATCTAGGTGAAGATCCTTTTTGATAAT |
| | | CTCATGACCAAAATCCCTTAACGTGAGTTTTCGTTCCACTGAGCGTCAGA |
| | | CCCC |
| 145 | <#3022 | GTAGAAAAGATCAAAGGATCTTCTTGAGATCCTTTTTTTCTGCGCGTAATC |
| | pAAV_FOXP3. | TGCTGCTTGCAAACAAAAAAACCACCGCTACCAGCGGTGGTTTGTTTGCC |
| | 025_MND.FOX | GGATCAAGAGCTACCAACTCTTTTTCCGAAGGTAACTGGCTTCAGCAGAG |
| | P3geneartCDS.P | CGCAGATACCAAATACTGTCCTTCTAGTGTAGCCGTAGTTAGGCCACCAC |
| | 2A.LNGFR.WP | TTCAAGAACTCTGTAGCACCGCCTACATACCTCGCTCTGCTAATCCTGTTA |
| | REc3.pA_025> | CCAGTGGCTGCTGCCAGTGGCGATAAGTCGTGTCTTACCGGGTTGGACTC |
| | | AAGACGATAGTTACCGGATAAGGCGCAGCGGTCGGGCTGAACGGGGGGT |
| | | TCGTGCACACAGCCCAGCTTGGAGCGAACGACCTACACCGAACTGAGAT |
| | | ACCTACAGCGTGAGCTATGAGAAAGCGCCACGCTTCCCGAAGGGAGAAA |
| | | GGCGGACAGGTATCCGGTAAGCGGCAGGGTCGGAACAGGAGAGCGCACG |
| | | AGGGAGCTTCCAGGGGGAAACGCCTGGTATCTTTATAGTCCTGTCGGGTT |
| | | TCGCCACCTCTGACTTGAGCGTCGATTTTTGTGATGCTCGTCAGGGGGGC |
| | | GGAGCCTATGGAAAAACGCCAGCAACGCGGCCTTTTTACGGTTCCTGGCC |
| | | TTTTGCTGGCCTTTTGCTCACATGTTCTTTCCTGCGTTATCCCCTGATTCTG |
| | | TGGATAACCGTATTACCGCCTTTGAGTGAGCTGATACCGCTCGCCGCAGC |
| | | CGAACGACCGAGCGCAGCGAGTCAGTGAGCGAGGAAGCGGAAGAGCGC |
| | | CCAATACGCAAACCGCCTCTCCCCGCGCGTTGGCCGATTCATTAATGCAG |
| | | CTGCGCGCTCGCTCGCTCACTGAGGCCGCCCGGGCAAAGCCCGGGCGTCG |
| | | GGCGACCTTTGGTCGCCCGGCCTCAGTGAGCGAGCGAGCGCGCAGAGAG |
| | | GGAGTGGCCAACTCCATCACTAGGGGTTCCTTGTAGTTAATGATTAACCC |
| | | GCCATGCTACTTATCTACGTAGCGGCCGCTGCTAGCGTGGGCAGGCAAGC |
| | | CAGGTGCTGGACCTCTGCACGTGGGGCATGTGTGGGTATGTACATGTACC |
| | | TGTGTTCTTGGTGTGTGTGTGTGTGTGTGTGTGTGTGTGTGTCTAGAGCTG |
| | | GGGTGCAACTATGGGGCCCCTCGGGACATGTCCCAGCCAATGCCTGCTTT |
| | | GACCAGAGGAGTGTCCACGTGGCTCAGGTGGTCGAGTATCTCATACCGCC |
| | | CTAGCACACGTGTGACTCCTTTCCCCTATTGTCTACACGCGTAGGAACAG |
| | | AGAAACAGGAGAATATGGGCCAAACAGGATATCTGTGGTAAGCAGTTCC |
| | | TGCCCCGGCTCAGGGCCAAGAACAGTTGGAACAGCAGAATATGGGCCAA |
| | | ACAGGATATCTGTGGTAAGCAGTTCCTGCCCCGGCTCAGGGCCAAGAACA |
| | | GATGGTCCCCAGATGCGGTCCCGCCCTCAGCAGTTTCTAGAGAACCATCA |
| | | GATGTTTCCAGGGTGCCCCAAGGACCTGAAATGACCCTGTGCCTTATTTG |
| | | AACTAACCAATCAGTTCGCTTCTCGCTTCTGTTCGCGCGCTTCTGCTCCCC |
| | | GAGCTCTATATAAGCAGAGCTCGTTTAGTGAACCGTCAGATCGCCTGGAG |
| | | ACGCCATCCACGCTGTTTTGACTTCCATAGAAGGATCTCGAGGCCACCAT |
| | | GCCTAATCCTCGGCCTGGAAAGCCTAGCGCTCCTTCTCTTGCTCTGGGACC |
| | | TTCTCCTGGCGCCTCTCCATCTTGGAGAGCCGCTCCTAAAGCCAGCGATCT |
| | | GCTGGGAGCTAGAGGACCTGGCGGCACATTTCAGGGCAGAGATCTTAGA |
| | | GGCGGAGCCCACGCTAGCTCCTCCAGCCTTAATCCTATGCCTCCTAGCCA |
| | | GCTCCAGCTGCCTACACTGCCTCTGGTTATGGTGGCTCCTAGCGGAGCTA |
| | | GACTGGGCCCTCTGCCTCATCTGCAAGCTCTGCTGCAGGACAGACCCCAC |
| | | TTCATGCACCAGCTGAGCACCGTGGATGCCCACGCAAGAACACCTGTGCT |
| | | GCAGGTTCACCCTCTGGAATCCCCAGCCATGATCAGCCTGACACCTCCAA |
| | | CAACAGCCACCGGCGTGTTCAGCCTGAAAGCCAGACCTGGACTGCCTCCT |
| | | GGCATCAATGTGGCCAGCCTGGAATGGGTGTCCAGAGAACCTGCTCTGCT |
| | | GTGCACATTCCCCAATCCAAGCGCTCCCAGAAAGGACAGCACACTGTCTG |
| | | CCGTGCCTCAGAGCAGCTATCCCCTGCTTGCTAACGGCGTGTGCAAGTGG |
| | | CCTGGATGCGAGAAGGTGTTCGAGGAACCCGAGGACTTCCTGAAGCACT |
| | | GCCAGGCCGATCATCTGCTGGACGAGAAAGGCAGAGCCCAGTGTCTGCT |
| | | CCAGCGCGAGATGGTGCAGTCTCTGGAACAGCAGCTGGTCCTGGAAAAA |
| | | GAAAAGCTGAGCGCCATGCAGGCCCACCTGGCCGGAAAAATGGCCCTGA |
| | | CAAAGGCCAGCAGCGTGGCCTCTTCTGATAAGGGCAGCTGCTGCATTGTG |
| | | GCCGCTGGATCTCAGGGACCTGTGGTTCCTGCTTGGAGCGGACCTAGAGA |
| | | GGCCCCTGATTCTCTGTTTGCCGTGCGGAGACACCTGTGGGGCTCTCACG |
| | | GCAACTCTACTTTCCCCGAGTTCCTGCACAACATGGACTACTTCAAGTTCC |
| | | ACAACATGCGGCCTCCATTCACCTACGCCACACTGATCAGATGGGCCATT |
| | | CTGGAAGCCCCTGAGAAGCAGAGAACCCTGAACGAGATCTACCACTGGT |
| | | TTACCCGGATGTTCGCCTTCTTCCGGAATCACCCTGCCACCTGGAAGAAC |
| | | GCCATCCGGCACAATCTGAGCCTGCACAAGTGCTTCGTGCGCGTGGAATC |
| | | TGAGAAAGGCGCCGTGTGGACAGTGGACGAGCTGGAATTCAGAAAGAAG |
| | | AGAAGCCAGCGGCCTAGCCGGTGCAGCAATCCTACACCTGGACCTGGAA |
| | | GCGGAGCGACTAACTTCAGCCTGCTGAAGCAGGCCGGAGATGTGGAGGA |
| | | AAACCCTGGACCGATGGGGGCAGGTGCCACCGGACGAGCCATGGACGGG |
| | | CCGCGCCTGCTGCTGTTGCTGCTTCTGGGGGTGTCCCTTGGAGGTGCCAA |
| | | GGAGGCATGCCCCACAGGCCTGTACACACACAGCGGTGAGTGCTGCAAA |
| | | GCCTGCAACCTGGGCGAGGGTGTGGCCCAGCCTTGTGGAGCCAACCAGA |
| | | CCGTGTGTGAGCCCTGCCTGGACAGCGTGACGTTCTCCGACGTGGTGAGC |
| | | GCGACCGAGCCGTGCAAGCCGTGCACCGAGTGCGTGGGGCTCCAGAGCA |
| | | TGTCGGCGCCGTGCGTGGAGGCCGACGACGCCGTGTGCCGCTGCGCCTAC |
| | | GGCTACTACCAGGATGAGACGACTGGGCGCTGCGAGGCGTGCCGCGTGT |
| | | GCGAGGCGGGCTCGGGCCTCGTGTTCTCCTGCCAGGACAAGCAGAACAC |
| | | CGTGTGCGAGGAGTGCCCCGACGGCACGTATTCCGACGAGGCCAACCAC |
| | | GTGGACCCGTGCCTGCCCTGCACCGTGTGCGAGGACACCGAGCGCCAGCT |
| | | CCGCGAGTGCACACGCTGGGCCGACGCCGAGTGCGAGGAGATCCCTGGC |
| | | CGTTGGATTACACGGTCCACACCCCCAGAGGGCTCGGACAGCACAGCCCC |
| | | CAGCACCCAGGAGCCTGAGGCACCTCCAGAACAAGACCTCATAGCCAGC |
| | | ACGGTGGCAGGTGTGGTGACCACAGTGATGGGCAGCTCCCAGCCCGTGG |
| | | TGACCCGAGGCACCACCGACAACCTCATCCCTGTCTATTGCTCCATCCTG |
| | | GCTGCTGTGGTTGTGGGTCTTGTGGCCTACATAGCCTTCAAGAGGTGAAA |
| | | GCTTGATAATCAACCTCTGGATTACAAAATTTGTGAAAGATTGACTGGTA |
| | | TTCTTAACTATGTTGCTCCTTTTACGCTATGTGGATACGCTGCTTTAATGC |
| | | CTTTGTATCATGCTATTGCTTCCCGTATGGCTTTCATTTTCTCCTCCTTGTA |
| | | TAAATCCTGGTTAGTTCTTGCCACGGCGGAACTCATCGCCGCCTGCCTTGC |
| | | CCGCTGCTGGACAGGGGCTCGGCTGTTGGGCACTGACAATTCCGTGGGTC |
| | | GACTGCTTTATTTGTGAAATTTGTGATGCTATTGCTTTATTTGTAACCATT |
| | | ATAAGCTGCAATAAACAAGTTAACAACAACAATTGCATTCATTTTATGTT |
| | | TCAGGTTCAGGGGGAGATGTGGGAGGTTTTTTAAAGCACTAGTGTGAGGC |
| | | CCTGGGCCCAGGATGGGGCAGGCAGGGTGGGGTACCTGGACCTACAGGT |
| | | GCCGACCTTTACTGTGGCACTGGGCGGGAGGGGGGCTGGCTGGGGCACA |
| | | GGAAGTGGTTTCTGGGTCCCAGGCAAGTCTGTGACTTATGCAGATGTTGC |
| | | AGGGCCAAGAAAATCCCCACCTGCCAGGCCTCAGAGATTGGAGGCTCTC |
| | | CCCGACCTCCCAATCCCTGTCTCAGGAGAGGAGGAGGCCGTGGATCCTAC |
| | | GTAGATAAGTAGCATGGCGGGTTAATCATTAACTACAAGGAACCCCTAGT |
| | | GATGGAGTTGGCCACTCCCTCTCTGCGCGCTCGCTCGCTCACTGAGGCCG |
| | | GGCGACCAAAGGTCGCCCGACGCCCGGGCTTTGCCCGGGCGGCCTCAGT |
| | | GAGCGAGCGAGCGCGCCAGCTGGCGTAATAGCGAAGAGGCCCGCACCGA |
| | | TCGCCCTTCCCAACAGTTGCGCAGCCTGAATGGCGAATGGCGATTCCGTT |
| | | GCAATGGCTGGCGGTAATATTGTTCTGGATATTACCAGCAAGGCCGATAG |
| | | TTTGAGTTCTTCTACTCAGGCAAGTGATGTTATTACTAATCAAAGAAGTAT |
| | | TGCGACAACGGTTAATTTGCGTGATGGACAGACTCTTTTACTCGGTGGCC |
| | | TCACTGATTATAAAAACACTTCTCAGGATTCTGGCGTACCGTTCCTGTCTA |
| | | AAATCCCTTTAATCGGCCTCCTGTTTAGCTCCCGCTCTGATTCTAACGAGG |
| | | AAAGCACGTTATACGTGCTCGTCAAAGCAACCATAGTACGCGCCCTGTAG |
| | | CGGCGCATTAAGCGCGGCGGGTGTGGTGGTTACGCGCAGCGTGACCGCT |
| | | ACACTTGCCAGCGCCCTAGCGCCCGCTCCTTTCGCTTTCTTCCCTTCCTTTC |
| | | TCGCCACGTTCGCCGGCTTTCCCCGTCAAGCTCTAAATCGGGGGCTCCCTT |
| | | TAGGGTTCCGATTTAGTGCTTTACGGCACCTCGACCCCAAAAAACTTGAT |
| | | TAGGGTGATGGTTCACGTAGTGGGCCATCGCCCTGATAGACGGTTTTTCG |
| | | CCCTTTGACGTTGGAGTCCACGTTCTTTAATAGTGGACTCTTGTTCCAAAC |
| | | TGGAACAACACTCAACCCTATCTCGGTCTATTCTTTTGATTTATAAGGGAT |
| | | TTTGCCGATTTCGGCCTATTGGTTAAAAAATGAGCTGATTTAACAAAAAT |
| | | TTAACGCGAATTTTAACAAAATATTAACGTTTACAATTTAAATATTTGCTT |
| | | ATACAATCTTCCTGTTTTTGGGGCTTTTCTGATTATCAACCGGGGTACATA |
| | | TGATTGACATGCTAGTTTTACGATTACCGTTCATCGATTCTCTTGTTTGCT |
| | | CCAGACTCTCAGGCAATGACCTGATAGCCTTTGTAGAGACCTCTCAAAAA |
| | | TAGCTACCCTCTCCGGCATGAATTTATCAGCTAGAACGGTTGAATATCAT |
| | | ATTGATGGTGATTTGACTGTCTCCGGCCTTTCTCACCCGTTTGAATCTTTA |
| | | CCTACACATTACTCAGGCATTGCATTTAAAATATATGAGGGTTCTAAAAA |
| | | TTTTTATCCTTGCGTTGAAATAAAGGCTTCTCCCGCAAAAGTATTACAGG |
| | | GTCATAATGTTTTTGGTACAACCGATTTAGCTTTATGCTCTGAGGCTTTAT |
| | | TGCTTAATTTTGCTAATTCTTTGCCTTGCCTGTATGATTTATTGGATGTTGG |
| | | AATCGCCTGATGCGGTATTTTCTCCTTACGCATCTGTGCGGTATTTCACAC |
| | | CGCATATGGTGCACTCTCAGTACAATCTGCTCTGATGCCGCATAGTTAAG |
| | | CCAGCCCCGACACCCGCCAACACCCGCTGACGCGCCCTGACGGGCTTGTC |
| | | TGCTCCCGGCATCCGCTTACAGACAAGCTGTGACCGTCTCCGGGAGCTGC |
| | | ATGTGTCAGAGGTTTTCACCGTCATCACCGAAACGCGCGAGACGAAAGG |
| | | GCCTCGTGATACGCCTATTTTTATAGGTTAATGTCATGATAATAATGGTTT |
| | | CTTAGACGTCAGGTGGCACTTTTCGGGGAAATGTGCGCGGAACCCCTATT |
| | | TGTTTATTTTTCTAAATACATTCAAATATGTATCCGCTCATGAGACAATAA |
| | | CCCTGATAAATGCTTCAATAATATTGAAAAAGGAAGAGTATGAGTATTCA |
| | | ACATTTCCGTGTCGCCCTTATTCCCTTTTTTGCGGCATTTTGCCTTCCTGTT |
| | | TTTGCTCACCCAGAAACGCTGGTGAAAGTAAAAGATGCTGAAGATCAGTT |
| | | GGGTGCACGAGTGGGTTACATCGAACTGGATCTCAACAGCGGTAAGATC |
| | | CTTGAGAGTTTTCGCCCCGAAGAACGTTTTCCAATGATGAGCACTTTTAA |
| | | AGTTCTGCTATGTGGCGCGGTATTATCCCGTATTGACGCCGGGCAAGAGC |
| | | AACTCGGTCGCCGCATACACTATTCTCAGAATGACTTGGTTGAGTACTCA |
| | | CCAGTCACAGAAAAGCATCTTACGGATGGCATGACAGTAAGAGAATTAT |
| | | GCAGTGCTGCCATAACCATGAGTGATAACACTGCGGCCAACTTACTTCTG |
| | | ACAACGATCGGAGGACCGAAGGAGCTAACCGCTTTTTTGCACAACATGG |
| | | GGGATCATGTAACTCGCCTTGATCGTTGGGAACCGGAGCTGAATGAAGCC |
| | | ATACCAAACGACGAGCGTGACACCACGATGCCTGTAGCAATGGCAACAA |
| | | CGTTGCGCAAACTATTAACTGGCGAACTACTTACTCTAGCTTCCCGGCAA |
| | | CAATTAATAGACTGGATGGAGGCGGATAAAGTTGCAGGACCACTTCTGC |
| | | GCTCGGCCCTTCCGGCTGGCTGGTTTATTGCTGATAAATCTGGAGCCGGT |
| | | GAGCGTGGGTCTCGCGGTATCATTGCAGCACTGGGGCCAGATGGTAAGCC |
| | | CTCCCGTATCGTAGTTATCTACACGACGGGGAGTCAGGCAACTATGGATG |
| | | AACGAAATAGACAGATCGCTGAGATAGGTGCCTCACTGATTAAGCATTG |
| | | GTAACTGTCAGACCAAGTTTACTCATATATACTTTAGATTGATTTAAAACT |
| | | TCATTTTTAATTTAAAAGGATCTAGGTGAAGATCCTTTTTGATAATCTCAT |
| | | GACCAAAATCCCTTAACGTGAGTTTTCGTTCCACTGAGCGTCAGACCCC |
| 146 | <#3023 | GTAGAAAAGATCAAAGGATCTTCTTGAGATCCTTTTTTTCTGCGCGTAATC |
| | pAAV_FOXP3. | TGCTGCTTGCAAACAAAAAAACCACCGCTACCAGCGGTGGTTTGTTTGCC |
| | 045_MND.FOX | GGATCAAGAGCTACCAACTCTTTTTCCGAAGGTAACTGGCTTCAGCAGAG |
| | P3geneartCDS.P | CGCAGATACCAAATACTGTCCTTCTAGTGTAGCCGTAGTTAGGCCACCAC |
| | 2A.LNGFR.WP | TTCAAGAACTCTGTAGCACCGCCTACATACCTCGCTCTGCTAATCCTGTTA |
| | REc3.pA_06> | CCAGTGGCTGCTGCCAGTGGCGATAAGTCGTGTCTTACCGGGTTGGACTC |
| | | AAGACGATAGTTACCGGATAAGGCGCAGCGGTCGGGCTGAACGGGGGGT |
| | | TCGTGCACACAGCCCAGCTTGGAGCGAACGACCTACACCGAACTGAGAT |
| | | ACCTACAGCGTGAGCTATGAGAAAGCGCCACGCTTCCCGAAGGGAGAAA |
| | | GGCGGACAGGTATCCGGTAAGCGGCAGGGTCGGAACAGGAGAGCGCACG |
| | | AGGGAGCTTCCAGGGGGAAACGCCTGGTATCTTTATAGTCCTGTCGGGTT |
| | | TCGCCACCTCTGACTTGAGCGTCGATTTTTGTGATGCTCGTCAGGGGGGC |
| | | GGAGCCTATGGAAAAACGCCAGCAACGCGGCCTTTTTACGGTTCCTGGCC |
| | | TTTTGCTGGCCTTTTGCTCACATGTTCTTTCCTGCGTTATCCCCTGATTCTG |
| | | TGGATAACCGTATTACCGCCTTTGAGTGAGCTGATACCGCTCGCCGCAGC |
| | | CGAACGACCGAGCGCAGCGAGTCAGTGAGCGAGGAAGCGGAAGAGCGC |
| | | CCAATACGCAAACCGCCTCTCCCCGCGCGTTGGCCGATTCATTAATGCAG |
| | | CTGCGCGCTCGCTCGCTCACTGAGGCCGCCCGGGCAAAGCCCGGGCGTCG |
| | | GGCGACCTTTGGTCGCCCGGCCTCAGTGAGCGAGCGAGCGCGCAGAGAG |
| | | GGAGTGGCCAACTCCATCACTAGGGGTTCCTTGTAGTTAATGATTAACCC |
| | | GCCATGCTACTTATCTACGTAGCGGCCGCAGTCCATGCCTAGTCACTGGG |
| | | GCAAAATAGGACTCCGAGGAGAAAGTCCGAGACCAGCTCCGGCAAGATG |
| | | AGCAAACACAGCCTGTGCAGGGTGCAGGGAGGGCTAGAGGCCTGAGGCT |
| | | TGAAACAGCTCTCAAGTGGAGGGGGAAACAACCATTGCCCTCATAGAGG |
| | | ACACATCCACACCAGGGCTGTGCTAGCGTGGGCAGGCAAGCCAGGTGCT |
| | | GGACCTCTGCACGTGGGGCATGTGTGGGTATGTACATGTACCTGTGTTCT |
| | | TGGTGTGTGTGTGTGTGTGTGTGTGTGTGTGTGTCTAGAGCTGGGGTGCA |
| | | ACTATGGGGCCCCTCGGGACATGTCCCAGCCAATGCCTGCTTTGACCAGA |
| | | GGAGTGTCCACGTGGCTCAGGTGGTCGAGTATCTCATACCGCCCTAGCAC |
| | | ACGTGTGACTCCTTTCCCCTATTGTCTACACGCGTAGGAACAGAGAAACA |
| | | GGAGAATATGGGCCAAACAGGATATCTGTGGTAAGCAGTTCCTGCCCCG |
| | | GCTCAGGGCCAAGAACAGTTGGAACAGCAGAATATGGGCCAAACAGGAT |
| | | ATCTGTGGTAAGCAGTTCCTGCCCCGGCTCAGGGCCAAGAACAGATGGTC |
| | | CCCAGATGCGGTCCCGCCCTCAGCAGTTTCTAGAGAACCATCAGATGTTT |
| | | CCAGGGTGCCCCAAGGACCTGAAATGACCCTGTGCCTTATTTGAACTAAC |
| | | CAATCAGTTCGCTTCTCGCTTCTGTTCGCGCGCTTCTGCTCCCCGAGCTCT |
| | | ATATAAGCAGAGCTCGTTTAGTGAACCGTCAGATCGCCTGGAGACGCCAT |
| | | CCACGCTGTTTTGACTTCCATAGAAGGATCTCGAGGCCACCATGCCTAAT |
| | | CCTCGGCCTGGAAAGCCTAGCGCTCCTTCTCTTGCTCTGGGACCTTCTCCT |
| | | GGCGCCTCTCCATCTTGGAGAGCCGCTCCTAAAGCCAGCGATCTGCTGGG |
| | | AGCTAGAGGACCTGGCGGCACATTTCAGGGCAGAGATCTTAGAGGCGGA |
| | | GCCCACGCTAGCTCCTCCAGCCTTAATCCTATGCCTCCTAGCCAGCTCCAG |
| | | CTGCCTACACTGCCTCTGGTTATGGTGGCTCCTAGCGGAGCTAGACTGGG |
| | | CCCTCTGCCTCATCTGCAAGCTCTGCTGCAGGACAGACCCCACTTCATGC |
| | | ACCAGCTGAGCACCGTGGATGCCCACGCAAGAACACCTGTGCTGCAGGTT |
| | | CACCCTCTGGAATCCCCAGCCATGATCAGCCTGACACCTCCAACAACAGC |
| | | CACCGGCGTGTTCAGCCTGAAAGCCAGACCTGGACTGCCTCCTGGCATCA |
| | | ATGTGGCCAGCCTGGAATGGGTGTCCAGAGAACCTGCTCTGCTGTGCACA |
| | | TTCCCCAATCCAAGCGCTCCCAGAAAGGACAGCACACTGTCTGCCGTGCC |
| | | TCAGAGCAGCTATCCCCTGCTTGCTAACGGCGTGTGCAAGTGGCCTGGAT |
| | | GCGAGAAGGTGTTCGAGGAACCCGAGGACTTCCTGAAGCACTGCCAGGC |
| | | CGATCATCTGCTGGACGAGAAAGGCAGAGCCCAGTGTCTGCTCCAGCGC |
| | | GAGATGGTGCAGTCTCTGGAACAGCAGCTGGTCCTGGAAAAAGAAAAGC |
| | | TGAGCGCCATGCAGGCCCACCTGGCCGGAAAAATGGCCCTGACAAAGGC |
| | | CAGCAGCGTGGCCTCTTCTGATAAGGGCAGCTGCTGCATTGTGGCCGCTG |
| | | GATCTCAGGGACCTGTGGTTCCTGCTTGGAGCGGACCTAGAGAGGCCCCT |
| | | GATTCTCTGTTTGCCGTGCGGAGACACCTGTGGGGCTCTCACGGCAACTC |
| | | TACTTTCCCCGAGTTCCTGCACAACATGGACTACTTCAAGTTCCACAACAT |
| | | GCGGCCTCCATTCACCTACGCCACACTGATCAGATGGGCCATTCTGGAAG |
| | | CCCCTGAGAAGCAGAGAACCCTGAACGAGATCTACCACTGGTTTACCCGG |
| | | ATGTTCGCCTTCTTCCGGAATCACCCTGCCACCTGGAAGAACGCCATCCG |
| | | GCACAATCTGAGCCTGCACAAGTGCTTCGTGCGCGTGGAATCTGAGAAAG |
| | | GCGCCGTGTGGACAGTGGACGAGCTGGAATTCAGAAAGAAGAGAAGCCA |
| | | GCGGCCTAGCCGGTGCAGCAATCCTACACCTGGACCTGGAAGCGGAGCG |
| | | ACTAACTTCAGCCTGCTGAAGCAGGCCGGAGATGTGGAGGAAAACCCTG |
| | | GACCGATGGGGGCAGGTGCCACCGGACGAGCCATGGACGGGCCGCGCCT |
| | | GCTGCTGTTGCTGCTTCTGGGGGTGTCCCTTGGAGGTGCCAAGGAGGCAT |
| | | GCCCCACAGGCCTGTACACACACAGCGGTGAGTGCTGCAAAGCCTGCAA |
| | | CCTGGGCGAGGGTGTGGCCCAGCCTTGTGGAGCCAACCAGACCGTGTGTG |
| | | AGCCCTGCCTGGACAGCGTGACGTTCTCCGACGTGGTGAGCGCGACCGAG |
| | | CCGTGCAAGCCGTGCACCGAGTGCGTGGGGCTCCAGAGCATGTCGGCGC |
| | | CGTGCGTGGAGGCCGACGACGCCGTGTGCCGCTGCGCCTACGGCTACTAC |
| | | CAGGATGAGACGACTGGGCGCTGCGAGGCGTGCCGCGTGTGCGAGGCGG |
| | | GCTCGGGCCTCGTGTTCTCCTGCCAGGACAAGCAGAACACCGTGTGCGAG |
| | | GAGTGCCCCGACGGCACGTATTCCGACGAGGCCAACCACGTGGACCCGT |
| | | GCCTGCCCTGCACCGTGTGCGAGGACACCGAGCGCCAGCTCCGCGAGTGC |
| | | ACACGCTGGGCCGACGCCGAGTGCGAGGAGATCCCTGGCCGTTGGATTA |
| | | CACGGTCCACACCCCCAGAGGGCTCGGACAGCACAGCCCCCAGCACCCA |
| | | GGAGCCTGAGGCACCTCCAGAACAAGACCTCATAGCCAGCACGGTGGCA |
| | | GGTGTGGTGACCACAGTGATGGGCAGCTCCCAGCCCGTGGTGACCCGAG |
| | | GCACCACCGACAACCTCATCCCTGTCTATTGCTCCATCCTGGCTGCTGTGG |
| | | TTGTGGGTCTTGTGGCCTACATAGCCTTCAAGAGGTGAAAGCTTGATAAT |
| | | CAACCTCTGGATTACAAAATTTGTGAAAGATTGACTGGTATTCTTAACTA |
| | | TGTTGCTCCTTTTACGCTATGTGGATACGCTGCTTTAATGCCTTTGTATCA |
| | | TGCTATTGCTTCCCGTATGGCTTTCATTTTCTCCTCCTTGTATAAATCCTGG |
| | | TTAGTTCTTGCCACGGCGGAACTCATCGCCGCCTGCCTTGCCCGCTGCTGG |
| | | ACAGGGGCTCGGCTGTTGGGCACTGACAATTCCGTGGGTCGACTGCTTTA |
| | | TTTGTGAAATTTGTGATGCTATTGCTTTATTTGTAACCATTATAAGCTGCA |
| | | ATAAACAAGTTAACAACAACAATTGCATTCATTTTATGTTTCAGGTTCAG |
| | | GGGGAGATGTGGGAGGTTTTTTAAAGCACTAGTGTGAGGCCCTGGGCCCA |
| | | GGATGGGGCAGGCAGGGTGGGGTACCTGGACCTACAGGTGCCGACCTTT |
| | | ACTGTGGCACTGGGCGGGAGGGGGGCTGGCTGGGGCACAGGAAGTGGTT |
| | | TCTGGGTCCCAGGCAAGTCTGTGACTTATGCAGATGTTGCAGGGCCAAGA |
| | | AAATCCCCACCTGCCAGGCCTCAGAGATTGGAGGCTCTCCCCGACCTCCC |
| | | AATCCCTGTCTCAGGAGAGGAGGAGGCCGTATTGTAGTCCCATGAGCATA |
| | | GCTATGTGTCCCCATCCCCATGTGACAAGAGAAGAGGACTGGGGCCAAG |
| | | TAGGTGAGGTGACAGGGCTGAGGCCAGCTCTGCAACTTATTAGCTGTTTG |
| | | ATCTTTAAAAAGTTACTCGATCTCCATGAGCCTCAGTTTCCATACGTGTAA |
| | | AAGGGGGATGATCATAGCATCTACCATGTGGGCTTGCAGTGCAGAGTATT |
| | | TGAATTAGACACAGAACAGTGAGGATCAGGATGGCCTCTCACCCACCTGC |
| | | CTTTCTGCCCAGCTGCCCACACTGCCCCTAGTCATGGTGGCACCCTCCGG |
| | | GGCACGGCTGGGCCCCTTGCCCCACTTACAGGCACCGCGGCGCTACGTAG |
| | | ATAAGTAGCATGGCGGGTTAATCATTAACTACAAGGAACCCCTAGTGATG |
| | | GAGTTGGCCACTCCCTCTCTGCGCGCTCGCTCGCTCACTGAGGCCGGGCG |
| | | ACCAAAGGTCGCCCGACGCCCGGGCTTTGCCCGGGCGGCCTCAGTGAGC |
| | | GAGCGAGCGCGCCAGCTGGCGTAATAGCGAAGAGGCCCGCACCGATCGC |
| | | CCTTCCCAACAGTTGCGCAGCCTGAATGGCGAATGGCGATTCCGTTGCAA |
| | | TGGCTGGCGGTAATATTGTTCTGGATATTACCAGCAAGGCCGATAGTTTG |
| | | AGTTCTTCTACTCAGGCAAGTGATGTTATTACTAATCAAAGAAGTATTGC |
| | | GACAACGGTTAATTTGCGTGATGGACAGACTCTTTTACTCGGTGGCCTCA |
| | | CTGATTATAAAAACACTTCTCAGGATTCTGGCGTACCGTTCCTGTCTAAA |
| | | ATCCCTTTAATCGGCCTCCTGTTTAGCTCCCGCTCTGATTCTAACGAGGAA |
| | | AGCACGTTATACGTGCTCGTCAAAGCAACCATAGTACGCGCCCTGTAGCG |
| | | GCGCATTAAGCGCGGCGGGTGTGGTGGTTACGCGCAGCGTGACCGCTAC |
| | | ACTTGCCAGCGCCCTAGCGCCCGCTCCTTTCGCTTTCTTCCCTTCCTTTCTC |
| | | GCCACGTTCGCCGGCTTTCCCCGTCAAGCTCTAAATCGGGGGCTCCCTTTA |
| | | GGGTTCCGATTTAGTGCTTTACGGCACCTCGACCCCAAAAAACTTGATTA |
| | | GGGTGATGGTTCACGTAGTGGGCCATCGCCCTGATAGACGGTTTTTCGCC |
| | | CTTTGACGTTGGAGTCCACGTTCTTTAATAGTGGACTCTTGTTCCAAACTG |
| | | GAACAACACTCAACCCTATCTCGGTCTATTCTTTTGATTTATAAGGGATTT |
| | | TGCCGATTTCGGCCTATTGGTTAAAAAATGAGCTGATTTAACAAAAATTT |
| | | AACGCGAATTTTAACAAAATATTAACGTTTACAATTTAAATATTTGCTTAT |
| | | ACAATCTTCCTGTTTTTGGGGCTTTTCTGATTATCAACCGGGGTACATATG |
| | | ATTGACATGCTAGTTTTACGATTACCGTTCATCGATTCTCTTGTTTGCTCC |
| | | AGACTCTCAGGCAATGACCTGATAGCCTTTGTAGAGACCTCTCAAAAATA |
| | | GCTACCCTCTCCGGCATGAATTTATCAGCTAGAACGGTTGAATATCATAT |
| | | TGATGGTGATTTGACTGTCTCCGGCCTTTCTCACCCGTTTGAATCTTTACC |
| | | TACACATTACTCAGGCATTGCATTTAAAATATATGAGGGTTCTAAAAATT |
| | | TTTATCCTTGCGTTGAAATAAAGGCTTCTCCCGCAAAAGTATTACAGGGT |
| | | CATAATGTTTTTGGTACAACCGATTTAGCTTTATGCTCTGAGGCTTTATTG |
| | | CTTAATTTTGCTAATTCTTTGCCTTGCCTGTATGATTTATTGGATGTTGGA |
| | | ATCGCCTGATGCGGTATTTTCTCCTTACGCATCTGTGCGGTATTTCACACC |
| | | GCATATGGTGCACTCTCAGTACAATCTGCTCTGATGCCGCATAGTTAAGC |
| | | CAGCCCCGACACCCGCCAACACCCGCTGACGCGCCCTGACGGGCTTGTCT |
| | | GCTCCCGGCATCCGCTTACAGACAAGCTGTGACCGTCTCCGGGAGCTGCA |
| | | TGTGTCAGAGGTTTTCACCGTCATCACCGAAACGCGCGAGACGAAAGGG |
| | | CCTCGTGATACGCCTATTTTTATAGGTTAATGTCATGATAATAATGGTTTC |
| | | TTAGACGTCAGGTGGCACTTTTCGGGGAAATGTGCGCGGAACCCCTATTT |
| | | GTTTATTTTTCTAAATACATTCAAATATGTATCCGCTCATGAGACAATAAC |
| | | CCTGATAAATGCTTCAATAATATTGAAAAAGGAAGAGTATGAGTATTCAA |
| | | CATTTCCGTGTCGCCCTTATTCCCTTTTTTGCGGCATTTTGCCTTCCTGTTT |
| | | TTGCTCACCCAGAAACGCTGGTGAAAGTAAAAGATGCTGAAGATCAGTT |
| | | GGGTGCACGAGTGGGTTACATCGAACTGGATCTCAACAGCGGTAAGATC |
| | | CTTGAGAGTTTTCGCCCCGAAGAACGTTTTCCAATGATGAGCACTTTTAA |
| | | AGTTCTGCTATGTGGCGCGGTATTATCCCGTATTGACGCCGGGCAAGAGC |
| | | AACTCGGTCGCCGCATACACTATTCTCAGAATGACTTGGTTGAGTACTCA |
| | | CCAGTCACAGAAAAGCATCTTACGGATGGCATGACAGTAAGAGAATTAT |
| | | GCAGTGCTGCCATAACCATGAGTGATAACACTGCGGCCAACTTACTTCTG |
| | | ACAACGATCGGAGGACCGAAGGAGCTAACCGCTTTTTTGCACAACATGG |
| | | GGGATCATGTAACTCGCCTTGATCGTTGGGAACCGGAGCTGAATGAAGCC |
| | | ATACCAAACGACGAGCGTGACACCACGATGCCTGTAGCAATGGCAACAA |
| | | CGTTGCGCAAACTATTAACTGGCGAACTACTTACTCTAGCTTCCCGGCAA |
| | | CAATTAATAGACTGGATGGAGGCGGATAAAGTTGCAGGACCACTTCTGC |
| | | GCTCGGCCCTTCCGGCTGGCTGGTTTATTGCTGATAAATCTGGAGCCGGT |
| | | GAGCGTGGGTCTCGCGGTATCATTGCAGCACTGGGGCCAGATGGTAAGCC |
| | | CTCCCGTATCGTAGTTATCTACACGACGGGGAGTCAGGCAACTATGGATG |
| | | AACGAAATAGACAGATCGCTGAGATAGGTGCCTCACTGATTAAGCATTG |
| | | GTAACTGTCAGACCAAGTTTACTCATATATACTTTAGATTGATTTAAAACT |
| | | TCATTTTTAATTTAAAAGGATCTAGGTGAAGATCCTTTTTGATAATCTCAT |
| | | GACCAAAATCCCTTAACGTGAGTTTTCGTTCCACTGAGCGTCAGACCCC |
| 147 | <#3024 | GTAGAAAAGATCAAAGGATCTTCTTGAGATCCTTTTTTTCTGCGCGTAATC |
| | pAAV_FOXP3 | TGCTGCTTGCAAACAAAAAAACCACCGCTACCAGCGGTGGTTTGTTTGCC |
| | 045_MND- | GGATCAAGAGCTACCAACTCTTTTTCCGAAGGTAACTGGCTTCAGCAGAG |
| | FOXP3geneartC | CGCAGATACCAAATACTGTCCTTCTAGTGTAGCCGTAGTTAGGCCACCAC |
| | DS.P2A.LNGFR | TTCAAGAACTCTGTAGCACCGCCTACATACCTCGCTCTGCTAATCCTGTTA |
| | .WPRE6.pA_06 | CCAGTGGCTGCTGCCAGTGGCGATAAGTCGTGTCTTACCGGGTTGGACTC |
| | > | AAGACGATAGTTACCGGATAAGGCGCAGCGGTCGGGCTGAACGGGGGGT |
| | | TCGTGCACACAGCCCAGCTTGGAGCGAACGACCTACACCGAACTGAGAT |
| | | ACCTACAGCGTGAGCTATGAGAAAGCGCCACGCTTCCCGAAGGGAGAAA |
| | | GGCGGACAGGTATCCGGTAAGCGGCAGGGTCGGAACAGGAGAGCGCACG |
| | | AGGGAGCTTCCAGGGGGAAACGCCTGGTATCTTTATAGTCCTGTCGGGTT |
| | | TCGCCACCTCTGACTTGAGCGTCGATTTTTGTGATGCTCGTCAGGGGGGC |
| | | GGAGCCTATGGAAAAACGCCAGCAACGCGGCCTTTTTACGGTTCCTGGCC |
| | | TTTTGCTGGCCTTTTGCTCACATGTTCTTTCCTGCGTTATCCCCTGATTCTG |
| | | TGGATAACCGTATTACCGCCTTTGAGTGAGCTGATACCGCTCGCCGCAGC |
| | | CGAACGACCGAGCGCAGCGAGTCAGTGAGCGAGGAAGCGGAAGAGCGC |
| | | CCAATACGCAAACCGCCTCTCCCCGCGCGTTGGCCGATTCATTAATGCAG |
| | | CTGCGCGCTCGCTCGCTCACTGAGGCCGCCCGGGCAAAGCCCGGGCGTCG |
| | | GGCGACCTTTGGTCGCCCGGCCTCAGTGAGCGAGCGAGCGCGCAGAGAG |
| | | GGAGTGGCCAACTCCATCACTAGGGGTTCCTTGTAGTTAATGATTAACCC |
| | | GCCATGCTACTTATCTACGTAGCGGCCGCAGTCCATGCCTAGTCACTGGG |
| | | GCAAAATAGGACTCCGAGGAGAAAGTCCGAGACCAGCTCCGGCAAGATG |
| | | AGCAAACACAGCCTGTGCAGGGTGCAGGGAGGGCTAGAGGCCTGAGGCT |
| | | TGAAACAGCTCTCAAGTGGAGGGGGAAACAACCATTGCCCTCATAGAGG |
| | | ACACATCCACACCAGGGCTGTGCTAGCGTGGGCAGGCAAGCCAGGTGCT |
| | | GGACCTCTGCACGTGGGGCATGTGTGGGTATGTACATGTACCTGTGTTCT |
| | | TGGTGTGTGTGTGTGTGTGTGTGTGTGTGTGTGTCTAGAGCTGGGGTGCA |
| | | ACTATGGGGCCCCTCGGGACATGTCCCAGCCAATGCCTGCTTTGACCAGA |
| | | GGAGTGTCCACGTGGCTCAGGTGGTCGAGTATCTCATACCGCCCTAGCAC |
| | | ACGTGTGACTCCTTTCCCCTATTGTCTACACGCGTAGGAACAGAGAAACA |
| | | GGAGAATATGGGCCAAACAGGATATCTGTGGTAAGCAGTTCCTGCCCCG |
| | | GCTCAGGGCCAAGAACAGTTGGAACAGCAGAATATGGGCCAAACAGGAT |
| | | ATCTGTGGTAAGCAGTTCCTGCCCCGGCTCAGGGCCAAGAACAGATGGTC |
| | | CCCAGATGCGGTCCCGCCCTCAGCAGTTTCTAGAGAACCATCAGATGTTT |
| | | CCAGGGTGCCCCAAGGACCTGAAATGACCCTGTGCCTTATTTGAACTAAC |
| | | CAATCAGTTCGCTTCTCGCTTCTGTTCGCGCGCTTCTGCTCCCCGAGCTCT |
| | | ATATAAGCAGAGCTCGTTTAGTGAACCGTCAGATCGCCTGGAGACGCCAT |
| | | CCACGCTGTTTTGACTTCCATAGAAGGATCTCGAGGCCACCATGCCTAAT |
| | | CCTCGGCCTGGAAAGCCTAGCGCTCCTTCTCTTGCTCTGGGACCTTCTCCT |
| | | GGCGCCTCTCCATCTTGGAGAGCCGCTCCTAAAGCCAGCGATCTGCTGGG |
| | | AGCTAGAGGACCTGGCGGCACATTTCAGGGCAGAGATCTTAGAGGCGGA |
| | | GCCCACGCTAGCTCCTCCAGCCTTAATCCTATGCCTCCTAGCCAGCTCCAG |
| | | CTGCCTACACTGCCTCTGGTTATGGTGGCTCCTAGCGGAGCTAGACTGGG |
| | | CCCTCTGCCTCATCTGCAAGCTCTGCTGCAGGACAGACCCCACTTCATGC |
| | | ACCAGCTGAGCACCGTGGATGCCCACGCAAGAACACCTGTGCTGCAGGTT |
| | | CACCCTCTGGAATCCCCAGCCATGATCAGCCTGACACCTCCAACAACAGC |
| | | CACCGGCGTGTTCAGCCTGAAAGCCAGACCTGGACTGCCTCCTGGCATCA |
| | | ATGTGGCCAGCCTGGAATGGGTGTCCAGAGAACCTGCTCTGCTGTGCACA |
| | | TTCCCCAATCCAAGCGCTCCCAGAAAGGACAGCACACTGTCTGCCGTGCC |
| | | TCAGAGCAGCTATCCCCTGCTTGCTAACGGCGTGTGCAAGTGGCCTGGAT |
| | | GCGAGAAGGTGTTCGAGGAACCCGAGGACTTCCTGAAGCACTGCCAGGC |
| | | CGATCATCTGCTGGACGAGAAAGGCAGAGCCCAGTGTCTGCTCCAGCGC |
| | | GAGATGGTGCAGTCTCTGGAACAGCAGCTGGTCCTGGAAAAAGAAAAGC |
| | | TGAGCGCCATGCAGGCCCACCTGGCCGGAAAAATGGCCCTGACAAAGGC |
| | | CAGCAGCGTGGCCTCTTCTGATAAGGGCAGCTGCTGCATTGTGGCCGCTG |
| | | GATCTCAGGGACCTGTGGTTCCTGCTTGGAGCGGACCTAGAGAGGCCCCT |
| | | GATTCTCTGTTTGCCGTGCGGAGACACCTGTGGGGCTCTCACGGCAACTC |
| | | TACTTTCCCCGAGTTCCTGCACAACATGGACTACTTCAAGTTCCACAACAT |
| | | GCGGCCTCCATTCACCTACGCCACACTGATCAGATGGGCCATTCTGGAAG |
| | | CCCCTGAGAAGCAGAGAACCCTGAACGAGATCTACCACTGGTTTACCCGG |
| | | ATGTTCGCCTTCTTCCGGAATCACCCTGCCACCTGGAAGAACGCCATCCG |
| | | GCACAATCTGAGCCTGCACAAGTGCTTCGTGCGCGTGGAATCTGAGAAAG |
| | | GCGCCGTGTGGACAGTGGACGAGCTGGAATTCAGAAAGAAGAGAAGCCA |
| | | GCGGCCTAGCCGGTGCAGCAATCCTACACCTGGACCTGGAAGCGGAGCG |
| | | ACTAACTTCAGCCTGCTGAAGCAGGCCGGAGATGTGGAGGAAAACCCTG |
| | | GACCGATGGGGGCAGGTGCCACCGGACGAGCCATGGACGGGCCGCGCCT |
| | | GCTGCTGTTGCTGCTTCTGGGGGTGTCCCTTGGAGGTGCCAAGGAGGCAT |
| | | GCCCCACAGGCCTGTACACACACAGCGGTGAGTGCTGCAAAGCCTGCAA |
| | | CCTGGGCGAGGGTGTGGCCCAGCCTTGTGGAGCCAACCAGACCGTGTGTG |
| | | AGCCCTGCCTGGACAGCGTGACGTTCTCCGACGTGGTGAGCGCGACCGAG |
| | | CCGTGCAAGCCGTGCACCGAGTGCGTGGGGCTCCAGAGCATGTCGGCGC |
| | | CGTGCGTGGAGGCCGACGACGCCGTGTGCCGCTGCGCCTACGGCTACTAC |
| | | CAGGATGAGACGACTGGGCGCTGCGAGGCGTGCCGCGTGTGCGAGGCGG |
| | | GCTCGGGCCTCGTGTTCTCCTGCCAGGACAAGCAGAACACCGTGTGCGAG |
| | | GAGTGCCCCGACGGCACGTATTCCGACGAGGCCAACCACGTGGACCCGT |
| | | GCCTGCCCTGCACCGTGTGCGAGGACACCGAGCGCCAGCTCCGCGAGTGC |
| | | ACACGCTGGGCCGACGCCGAGTGCGAGGAGATCCCTGGCCGTTGGATTA |
| | | CACGGTCCACACCCCCAGAGGGCTCGGACAGCACAGCCCCCAGCACCCA |
| | | GGAGCCTGAGGCACCTCCAGAACAAGACCTCATAGCCAGCACGGTGGCA |
| | | GGTGTGGTGACCACAGTGATGGGCAGCTCCCAGCCCGTGGTGACCCGAG |
| | | GCACCACCGACAACCTCATCCCTGTCTATTGCTCCATCCTGGCTGCTGTGG |
| | | TTGTGGGTCTTGTGGCCTACATAGCCTTCAAGAGGTGAAAGCTTTCGACA |
| | | ATCAACCTCTGGATTACAAAATTTGTGAAAGATTGACTGGTATTCTTAAC |
| | | TATGTTGCTCCTTTTACGCTATGTGGATACGCTGCTTTAATGCCTTTGTAT |
| | | CATGCTATTGCTTCCCGTATGGCTTTCATTTTCTCCTCCTTGTATAAATCCT |
| | | GGTTGCTGTCTCTTTATGAGGAGTTGTGGCCCGTTGTCAGGCAACGTGGC |
| | | GTGGTGTGCACTGTGTTTGCTGACGCAACCCCCACTGGTTGGGGCATTGC |
| | | CACCACCTGTCAGCTCCTTTCCGGGACTTTCGCTTTCCCCCTCCCTATTGC |
| | | CACGGCGGAACTCATCGCCGCCTGCCTTGCCCGCTGCTGGACAGGGGCTC |
| | | GGCTGTTGGGCACTGACAATTCCGTGGTGTTGTCGGGGAAGCTGACGTCC |
| | | TTTCCATGGCTGCTCGCCTGTGTTGCCACCTGGATTCTGCGCGGGACGTCC |
| | | TTCTGCTACGTCCCTTCGGCCCTCAATCCAGCGGACCTTCCTTCCCGCGGC |
| | | CTGCTGCCGGCTCTGCGGCCTCTTCCGCGTCTTCGCCTTCGCCCTCAGACG |
| | | AGTCGGATCTCCCTTTGGGCCGCCTCCCCGCCTGGAGTCGACTGCTTTATT |
| | | TGTGAAATTTGTGATGCTATTGCTTTATTTGTAACCATTATAAGCTGCAAT |
| | | AAACAAGTTAACAACAACAATTGCATTCATTTTATGTTTCAGGTTCAGGG |
| | | GGAGATGTGGGAGGTTTTTTAAAGCACTAGTGTGAGGCCCTGGGCCCAGG |
| | | ATGGGGCAGGCAGGGTGGGGTACCTGGACCTACAGGTGCCGACCTTTACT |
| | | GTGGCACTGGGCGGGAGGGGGGCTGGCTGGGGCACAGGAAGTGGTTTCT |
| | | GGGTCCCAGGCAAGTCTGTGACTTATGCAGATGTTGCAGGGCCAAGAAA |
| | | ATCCCCACCTGCCAGGCCTCAGAGATTGGAGGCTCTCCCCGACCTCCCAA |
| | | TCCCTGTCTCAGGAGAGGAGGAGGCCGTATTGTAGTCCCATGAGCATAGC |
| | | TATGTGTCCCCATCCCCATGTGACAAGAGAAGAGGACTGGGGCCAAGTA |
| | | GGTGAGGTGACAGGGCTGAGGCCAGCTCTGCAACTTATTAGCTGTTTGAT |
| | | CTTTAAAAAGTTACTCGATCTCCATGAGCCTCAGTTTCCATACGTGTAAA |
| | | AGGGGGATGATCATAGCATCTACCATGTGGGCTTGCAGTGCAGAGTATTT |
| | | GAATTAGACACAGAACAGTGAGGATCAGGATGGCCTCTCACCCACCTGC |
| | | CTTTCTGCCCAGCTGCCCACACTGCCCCTAGTCATGGTGGCACCCTCCGG |
| | | GGCACGGCTGGGCCCCTTGCCCCACTTACAGGCACCGCGGCGCTACGTAG |
| | | ATAAGTAGCATGGCGGGTTAATCATTAACTACAAGGAACCCCTAGTGATG |
| | | GAGTTGGCCACTCCCTCTCTGCGCGCTCGCTCGCTCACTGAGGCCGGGCG |
| | | ACCAAAGGTCGCCCGACGCCCGGGCTTTGCCCGGGCGGCCTCAGTGAGC |
| | | GAGCGAGCGCGCCAGCTGGCGTAATAGCGAAGAGGCCCGCACCGATCGC |
| | | CCTTCCCAACAGTTGCGCAGCCTGAATGGCGAATGGCGATTCCGTTGCAA |
| | | TGGCTGGCGGTAATATTGTTCTGGATATTACCAGCAAGGCCGATAGTTTG |
| | | AGTTCTTCTACTCAGGCAAGTGATGTTATTACTAATCAAAGAAGTATTGC |
| | | GACAACGGTTAATTTGCGTGATGGACAGACTCTTTTACTCGGTGGCCTCA |
| | | CTGATTATAAAAACACTTCTCAGGATTCTGGCGTACCGTTCCTGTCTAAA |
| | | ATCCCTTTAATCGGCCTCCTGTTTAGCTCCCGCTCTGATTCTAACGAGGAA |
| | | AGCACGTTATACGTGCTCGTCAAAGCAACCATAGTACGCGCCCTGTAGCG |
| | | GCGCATTAAGCGCGGCGGGTGTGGTGGTTACGCGCAGCGTGACCGCTAC |
| | | ACTTGCCAGCGCCCTAGCGCCCGCTCCTTTCGCTTTCTTCCCTTCCTTTCTC |
| | | GCCACGTTCGCCGGCTTTCCCCGTCAAGCTCTAAATCGGGGGCTCCCTTTA |
| | | GGGTTCCGATTTAGTGCTTTACGGCACCTCGACCCCAAAAAACTTGATTA |
| | | GGGTGATGGTTCACGTAGTGGGCCATCGCCCTGATAGACGGTTTTTCGCC |
| | | CTTTGACGTTGGAGTCCACGTTCTTTAATAGTGGACTCTTGTTCCAAACTG |
| | | GAACAACACTCAACCCTATCTCGGTCTATTCTTTTGATTTATAAGGGATTT |
| | | TGCCGATTTCGGCCTATTGGTTAAAAAATGAGCTGATTTAACAAAAATTT |
| | | AACGCGAATTTTAACAAAATATTAACGTTTACAATTTAAATATTTGCTTAT |
| | | ACAATCTTCCTGTTTTTGGGGCTTTTCTGATTATCAACCGGGGTACATATG |
| | | ATTGACATGCTAGTTTTACGATTACCGTTCATCGATTCTCTTGTTTGCTCC |
| | | AGACTCTCAGGCAATGACCTGATAGCCTTTGTAGAGACCTCTCAAAAATA |
| | | GCTACCCTCTCCGGCATGAATTTATCAGCTAGAACGGTTGAATATCATAT |
| | | TGATGGTGATTTGACTGTCTCCGGCCTTTCTCACCCGTTTGAATCTTTACC |
| | | TACACATTACTCAGGCATTGCATTTAAAATATATGAGGGTTCTAAAAATT |
| | | TTTATCCTTGCGTTGAAATAAAGGCTTCTCCCGCAAAAGTATTACAGGGT |
| | | CATAATGTTTTTGGTACAACCGATTTAGCTTTATGCTCTGAGGCTTTATTG |
| | | CTTAATTTTGCTAATTCTTTGCCTTGCCTGTATGATTTATTGGATGTTGGA |
| | | ATCGCCTGATGCGGTATTTTCTCCTTACGCATCTGTGCGGTATTTCACACC |
| | | GCATATGGTGCACTCTCAGTACAATCTGCTCTGATGCCGCATAGTTAAGC |
| | | CAGCCCCGACACCCGCCAACACCCGCTGACGCGCCCTGACGGGCTTGTCT |
| | | GCTCCCGGCATCCGCTTACAGACAAGCTGTGACCGTCTCCGGGAGCTGCA |
| | | TGTGTCAGAGGTTTTCACCGTCATCACCGAAACGCGCGAGACGAAAGGG |
| | | CCTCGTGATACGCCTATTTTTATAGGTTAATGTCATGATAATAATGGTTTC |
| | | TTAGACGTCAGGTGGCACTTTTCGGGGAAATGTGCGCGGAACCCCTATTT |
| | | GTTTATTTTTCTAAATACATTCAAATATGTATCCGCTCATGAGACAATAAC |
| | | CCTGATAAATGCTTCAATAATATTGAAAAAGGAAGAGTATGAGTATTCAA |
| | | CATTTCCGTGTCGCCCTTATTCCCTTTTTTGCGGCATTTTGCCTTCCTGTTT |
| | | TTGCTCACCCAGAAACGCTGGTGAAAGTAAAAGATGCTGAAGATCAGTT |
| | | GGGTGCACGAGTGGGTTACATCGAACTGGATCTCAACAGCGGTAAGATC |
| | | CTTGAGAGTTTTCGCCCCGAAGAACGTTTTCCAATGATGAGCACTTTTAA |
| | | AGTTCTGCTATGTGGCGCGGTATTATCCCGTATTGACGCCGGGCAAGAGC |
| | | AACTCGGTCGCCGCATACACTATTCTCAGAATGACTTGGTTGAGTACTCA |
| | | CCAGTCACAGAAAAGCATCTTACGGATGGCATGACAGTAAGAGAATTAT |
| | | GCAGTGCTGCCATAACCATGAGTGATAACACTGCGGCCAACTTACTTCTG |
| | | ACAACGATCGGAGGACCGAAGGAGCTAACCGCTTTTTTGCACAACATGG |
| | | GGGATCATGTAACTCGCCTTGATCGTTGGGAACCGGAGCTGAATGAAGCC |
| | | ATACCAAACGACGAGCGTGACACCACGATGCCTGTAGCAATGGCAACAA |
| | | CGTTGCGCAAACTATTAACTGGCGAACTACTTACTCTAGCTTCCCGGCAA |
| | | CAATTAATAGACTGGATGGAGGCGGATAAAGTTGCAGGACCACTTCTGC |
| | | GCTCGGCCCTTCCGGCTGGCTGGTTTATTGCTGATAAATCTGGAGCCGGT |
| | | GAGCGTGGGTCTCGCGGTATCATTGCAGCACTGGGGCCAGATGGTAAGCC |
| | | CTCCCGTATCGTAGTTATCTACACGACGGGGAGTCAGGCAACTATGGATG |
| | | AACGAAATAGACAGATCGCTGAGATAGGTGCCTCACTGATTAAGCATTG |
| | | GTAACTGTCAGACCAAGTTTACTCATATATACTTTAGATTGATTTAAAACT |
| | | TCATTTTTAATTTAAAAGGATCTAGGTGAAGATCCTTTTTGATAATCTCAT |
| | | GACCAAAATCCCTTAACGTGAGTTTTCGTTCCACTGAGCGTCAGACCCC |
| 148 | <#1303 pAAV | CAGCTGCGCGCTCGCTCGCTCACTGAGGCCGCCCGGGCAAAGCCCGGGC |
| | FOXP3_0.9[MN | GTCGGGCGACCTTTGGTCGCCCGGCCTCAGTGAGCGAGCGAGCGCGCAG |
| | D-GFPki]1.6> | AGAGGGAGTGGCCAACTCCATCACTAGGGGTTCCTTGTAGTTAATGATTA |
| | | ACCCGCCATGCTACTTATCTACGCTCAAGAGACCCCATCTCTCCTCCTCTC |
| | | TGTCACTTGCCATGCTGGATCCGTGCATGATCACACTCCTGGACTCGCCTC |
| | | CTTGCCCTGAGATCCAGACCCCCGTATTCAGCTGCCCCCTCAGCTCCTCCA |
| | | CTCACATATTTAATGCCAGACTCTTCATGTCTATCTACACCTGCACTTTTG |
| | | CACCCAATCCAACTCCCCGCCATGTCCCCCATCTCAGGTAATGTCAGCTC |
| | | GGTCCTTCCAGCTGCTCAAGCTAAAACCCATGTCACTTTGACTCTCCCTCT |
| | | TGCCCACTACATCCAAGCTGCTAGCACTGCTCCTGATCCAGCTTCAGATT |
| | | AAGTCTCAGAATCTACCCACTTCTCGCCTTCTCCACTGCCACCAGCCCATT |
| | | CTGTGCCAGCATCATCACTTGCCAGGACTGTTACAATAGCCTCCTCACTA |
| | | GCCCCACTCACAGCAGCCAGATGAATCTTTTGAGTCCATGCCTAGTCACT |
| | | GGGGCAAAATAGGACTCCGAGGAGAAAGTCCGAGACCAGCTCCGGCAAG |
| | | ATGAGCAAACACAGCCTGTGCAGGGTGCAGGGAGGGCTAGAGGCCTGAG |
| | | GCTTGAAACAGCTCTCAAGTGGAGGGGGAAACAACCATTGCCCTCATAG |
| | | AGGACACATCCACACCAGGGCTGTGCTAGCGTGGGCAGGCAAGCCAGGT |
| | | GCTGGACCTCTGCACGTGGGGCATGTGTGGGTATGTACATGTACCTGTGT |
| | | TCTTGGTGTGTGTGTGTGTGTGTGTGTGTGTGTGTGTCTAGAGCTGGGGTG |
| | | CAACTATGGGGCCCCTCGGGACATGTCCCAGCCAATGCCTGCTTTGACCA |
| | | GAGGAGTGTCCACGTGGCTCAGGTGGTCGAGTATCTCATACCGCCCTAGC |
| | | ACACGTGTGACTCCTTTCCCCTATTGTCTACGAACAGAGAAACAGGAGAA |
| | | TATGGGCCAAACAGGATATCTGTGGTAAGCAGTTCCTGCCCCGGCTCAGG |
| | | GCCAAGAACAGTTGGAACAGCAGAATATGGGCCAAACAGGATATCTGTG |
| | | GTAAGCAGTTCCTGCCCCGGCTCAGGGCCAAGAACAGATGGTCCCCAGAT |
| | | GCGGTCCCGCCCTCAGCAGTTTCTAGAGAACCATCAGATGTTTCCAGGGT |
| | | GCCCCAAGGACCTGAAATGACCCTGTGCCTTATTTGAACTAACCAATCAG |
| | | TTCGCTTCTCGCTTCTGTTCGCGCGCTTCTGCTCCCCGAGCTCTATATAAG |
| | | CAGAGCTCGTTTAGTGAACCGTCAGATCGTCTACGCAGCCTGCCCTTGGA |
| | | CAAGGACCCGATGCCCAACCCCAGGCCTGTGAGCAAGGGCGAGGAGCTG |
| | | TTCACCGGGGTGGTGCCCATCCTGGTCGAGCTGGACGGCGACGTAAACGG |
| | | CCACAAGTTCAGCGTGTCCGGCGAGGGCGAGGGCGATGCCACCTACGGC |
| | | AAGCTGACCCTGAAGTTCATCTGCACCACCGGCAAGCTGCCCGTGCCCTG |
| | | GCCCACCCTCGTGACCACCCTGACCTACGGCGTGCAGTGCTTCAGCCGCT |
| | | ACCCCGACCACATGAAGCAGCACGACTTCTTCAAGTCCGCCATGCCCGAA |
| | | GGCTACGTCCAGGAGCGCACCATCTTCTTCAAGGACGACGGCAACTACAA |
| | | GACCCGCGCCGAGGTGAAGTTCGAGGGCGACACCCTGGTGAACCGCATC |
| | | GAGCTGAAGGGCATCGACTTCAAGGAGGACGGCAACATCCTGGGGCACA |
| | | AGCTGGAGTACAACTACAACAGCCACAACGTCTATATCATGGCCGACAA |
| | | GCAGAAGAACGGCATCAAGGTGAACTTCAAGATCCGCCACAACATCGAG |
| | | GACGGCAGCGTGCAGCTCGCCGACCACTACCAGCAGAACACCCCCATCG |
| | | GCGACGGCCCCGTGCTGCTGCCCGACAACCACTACCTGAGCACCCAGTCC |
| | | GCCCTGAGCAAAGACCCCAACGAGAAGCGCGATCACATGGTCCTGCTGG |
| | | AGTTCGTGACCGCCGCCGGGATCACTCTCGGCATGGACGAGCTGTACAAG |
| | | GGCAAGCCCTCGGCCCCTTCCTTGGCCCTTGGCCCATCCCCAGGAGCCTC |
| | | GCCCAGCTGGAGGGCTGCCCCTAAAGCAAGCGACCTGCTGGGGGCCCGG |
| | | GGCCCGGGTGGCACGTTCCAGGGCCGAGATCTTCGAGGCGGGGCCCATG |
| | | CCTCCTCTTCTTCCTTGAACCCCATGCCACCATCGCAGCTGCAGGTGAGGC |
| | | CCTGGGCCCAGGATGGGGCAGGCAGGGTGGGGTACCTGGACCTACAGGT |
| | | GCCGACCTTTACTGTGGCACTGGGCGGGAGGGGGGCTGGCTGGGGCACA |
| | | GGAAGTGGTTTCTGGGTCCCAGGCAAGTCTGTGACTTATGCAGATGTTGC |
| | | AGGGCCAAGAAAATCCCCACCTGCCAGGCCTCAGAGATTGGAGGCTCTC |
| | | CCCGACCTCCCAATCCCTGTCTCAGGAGAGGAGGAGGCCGTATTGTAGTC |
| | | CCATGAGCATAGCTATGTGTCCCCATCCCCATGTGACAAGAGAAGAGGAC |
| | | TGGGGCCAAGTAGGTGAGGTGACAGGGCTGAGGCCAGCTCTGCAACTTA |
| | | TTAGCTGTTTGATCTTTAAAAAGTTACTCGATCTCCATGAGCCTCAGTTTC |
| | | CATACGTGTAAAAGGGGGATGATCATAGCATCTACCATGTGGGCTTGCAG |
| | | TGCAGAGTATTTGAATTAGACACAGAACAGTGAGGATCAGGATGGCCTCT |
| | | CACCCACCTGCCTTTCTGCCCAGCTGCCCACACTGCCCCTAGTCATGGTGG |
| | | CACCCTCCGGGGCACGGCTGGGCCCCTTGCCCCACTTACAGGCACTCCTC |
| | | CAGGACAGGCCACATTTCATGCACCAGGTATGGACGGTGAATGGGCAGG |
| | | GAGGAGGGAGCAGGTGGGAGAACTGTGGGGAGGGGCCCCGAGTCAGGC |
| | | TGAACCACAGCCCACATGTGCCCCCCAGCTCTCAACGGTGGATGCCCACG |
| | | CCCGGACCCCTGTGCTGCAGGTGCACCCCCTGGAGAGCCCAGCCATGATC |
| | | AGCCTCACACCACCCACCACCGCCACTGGGGTCTTCTCCCTCAAGGCCCG |
| | | GCCTGGCCTCCCACCTGGTAACACCTCAGCCCGTACCCCATGGCTTCACA |
| | | GAACCCCCAAGTCCCCAGATCCTTGGCTGTGAGCAGTGTAGGCTATTCTG |
| | | AATTGCAGTACTCTGGGGGTCAAAGGTGTCAGGTCTCAGAGGCTTGGAAA |
| | | CTCCACCCTCCAAAAAACGTCAGGTGCAGAACCTTAAAGATGCAGAATGT |
| | | CAAAATCACAAAACCACAGAGCTTTACAAAGCTAGTCAAAATGTCAGCA |
| | | CCTGCGAATGGCCGTCTTTAAGCTTCTCTGCCAGAAGCCTGGGACTTTGG |
| | | GGACAGCAGAGCCCCCTGGGAGTCAGGGTTTTCGAGGCTCAGGAGGGTG |
| | | GGAAGCTCAAAATGAGAGGCCTTGTGGGCCAAGCTCCAGAGCCCAGCCC |
| | | ACAGCCTCCATAGGTGCCCTGTCCCCACCCACAGGGATCAACGTGGCCAG |
| | | CCTGGAATGGGTGTCCAGGGAGCCGGCACTGCTCTGCACCTTCCCAAATC |
| | | CCAGTGCACCCAGGAAGGACAGGTCAGTGGACAGGGCTGGGAAGGATCC |
| | | TCGCCCTCCTATCCGTAGATAAGTAGCATGGCGGGTTAATCATTAACTAC |
| | | AAGGAACCCCTAGTGATGGAGTTGGCCACTCCCTCTCTGCGCGCTCGCTC |
| | | GCTCACTGAGGCCGGGCGACCAAAGGTCGCCCGACGCCCGGGCTTTGCCC |
| | | GGGCGGCCTCAGTGAGCGAGCGAGCGCGCCAGCTGGCGTAATAGCGAAG |
| | | AGGCCCGCACCGATCGCCCTTCCCAACAGTTGCGCAGCCTGAATGGCGAA |
| | | TGGCGATTCCGTTGCAATGGCTGGCGGTAATATTGTTCTGGATATTACCA |
| | | GCAAGGCCGATAGTTTGAGTTCTTCTACTCAGGCAAGTGATGTTATTACT |
| | | AATCAAAGAAGTATTGCGACAACGGTTAATTTGCGTGATGGACAGACTCT |
| | | TTTACTCGGTGGCCTCACTGATTATAAAAACACTTCTCAGGATTCTGGCGT |
| | | ACCGTTCCTGTCTAAAATCCCTTTAATCGGCCTCCTGTTTAGCTCCCGCTC |
| | | TGATTCTAACGAGGAAAGCACGTTATACGTGCTCGTCAAAGCAACCATAG |
| | | TACGCGCCCTGTAGCGGCGCATTAAGCGCGGCGGGTGTGGTGGTTACGCG |
| | | CAGCGTGACCGCTACACTTGCCAGCGCCCTAGCGCCCGCTCCTTTCGCTTT |
| | | CTTCCCTTCCTTTCTCGCCACGTTCGCCGGCTTTCCCCGTCAAGCTCTAAA |
| | | TCGGGGGCTCCCTTTAGGGTTCCGATTTAGTGCTTTACGGCACCTCGACCC |
| | | CAAAAAACTTGATTAGGGTGATGGTTCACGTAGTGGGCCATCGCCCTGAT |
| | | AGACGGTTTTTCGCCCTTTGACGTTGGAGTCCACGTTCTTTAATAGTGGAC |
| | | TCTTGTTCCAAACTGGAACAACACTCAACCCTATCTCGGTCTATTCTTTTG |
| | | ATTTATAAGGGATTTTGCCGATTTCGGCCTATTGGTTAAAAAATGAGCTG |
| | | ATTTAACAAAAATTTAACGCGAATTTTAACAAAATATTAACGTTTACAAT |
| | | TTAAATATTTGCTTATACAATCTTCCTGTTTTTGGGGCTTTTCTGATTATCA |
| | | ACCGGGGTACATATGATTGACATGCTAGTTTTACGATTACCGTTCATCGA |
| | | TTCTCTTGTTTGCTCCAGACTCTCAGGCAATGACCTGATAGCCTTTGTAGA |
| | | GACCTCTCAAAAATAGCTACCCTCTCCGGCATGAATTTATCAGCTAGAAC |
| | | GGTTGAATATCATATTGATGGTGATTTGACTGTCTCCGGCCTTTCTCACCC |
| | | GTTTGAATCTTTACCTACACATTACTCAGGCATTGCATTTAAAATATATGA |
| | | GGGTTCTAAAAATTTTTATCCTTGCGTTGAAATAAAGGCTTCTCCCGCAA |
| | | AAGTATTACAGGGTCATAATGTTTTTGGTACAACCGATTTAGCTTTATGCT |
| | | CTGAGGCTTTATTGCTTAATTTTGCTAATTCTTTGCCTTGCCTGTATGATTT |
| | | ATTGGATGTTGGAATCGCCTGATGCGGTATTTTCTCCTTACGCATCTGTGC |
| | | GGTATTTCACACCGCATATGGTGCACTCTCAGTACAATCTGCTCTGATGCC |
| | | GCATAGTTAAGCCAGCCCCGACACCCGCCAACACCCGCTGACGCGCCCTG |
| | | ACGGGCTTGTCTGCTCCCGGCATCCGCTTACAGACAAGCTGTGACCGTCT |
| | | CCGGGAGCTGCATGTGTCAGAGGTTTTCACCGTCATCACCGAAACGCGCG |
| | | AGACGAAAGGGCCTCGTGATACGCCTATTTTTATAGGTTAATGTCATGAT |
| | | AATAATGGTTTCTTAGACGTCAGGTGGCACTTTTCGGGGAAATGTGCGCG |
| | | GAACCCCTATTTGTTTATTTTTCTAAATACATTCAAATATGTATCCGCTCA |
| | | TGAGACAATAACCCTGATAAATGCTTCAATAATATTGAAAAAGGAAGAG |
| | | TATGAGTATTCAACATTTCCGTGTCGCCCTTATTCCCTTTTTTGCGGCATTT |
| | | TGCCTTCCTGTTTTTGCTCACCCAGAAACGCTGGTGAAAGTAAAAGATGC |
| | | TGAAGATCAGTTGGGTGCACGAGTGGGTTACATCGAACTGGATCTCAACA |
| | | GCGGTAAGATCCTTGAGAGTTTTCGCCCCGAAGAACGTTTTCCAATGATG |
| | | AGCACTTTTAAAGTTCTGCTATGTGGCGCGGTATTATCCCGTATTGACGCC |
| | | GGGCAAGAGCAACTCGGTCGCCGCATACACTATTCTCAGAATGACTTGGT |
| | | TGAGTACTCACCAGTCACAGAAAAGCATCTTACGGATGGCATGACAGTA |
| | | AGAGAATTATGCAGTGCTGCCATAACCATGAGTGATAACACTGCGGCCA |
| | | ACTTACTTCTGACAACGATCGGAGGACCGAAGGAGCTAACCGCTTTTTTG |
| | | CACAACATGGGGGATCATGTAACTCGCCTTGATCGTTGGGAACCGGAGCT |
| | | GAATGAAGCCATACCAAACGACGAGCGTGACACCACGATGCCTGTAGCA |
| | | ATGGCAACAACGTTGCGCAAACTATTAACTGGCGAACTACTTACTCTAGC |
| | | TTCCCGGCAACAATTAATAGACTGGATGGAGGCGGATAAAGTTGCAGGA |
| | | CCACTTCTGCGCTCGGCCCTTCCGGCTGGCTGGTTTATTGCTGATAAATCT |
| | | GGAGCCGGTGAGCGTGGGTCTCGCGGTATCATTGCAGCACTGGGGCCAG |
| | | ATGGTAAGCCCTCCCGTATCGTAGTTATCTACACGACGGGGAGTCAGGCA |
| | | ACTATGGATGAACGAAATAGACAGATCGCTGAGATAGGTGCCTCACTGA |
| | | TTAAGCATTGGTAACTGTCAGACCAAGTTTACTCATATATACTTTAGATTG |
| | | ATTTAAAACTTCATTTTTAATTTAAAAGGATCTAGGTGAAGATCCTTTTTG |
| | | ATAATCTCATGACCAAAATCCCTTAACGTGAGTTTTCGTTCCACTGAGCGT |
| | | CAGACCCCGTAGAAAAGATCAAAGGATCTTCTTGAGATCCTTTTTTTCTG |
| | | CGCGTAATCTGCTGCTTGCAAACAAAAAAACCACCGCTACCAGCGGTGGT |
| | | TTGTTTGCCGGATCAAGAGCTACCAACTCTTTTTCCGAAGGTAACTGGCTT |
| | | CAGCAGAGCGCAGATACCAAATACTGTCCTTCTAGTGTAGCCGTAGTTAG |
| | | GCCACCACTTCAAGAACTCTGTAGCACCGCCTACATACCTCGCTCTGCTA |
| | | ATCCTGTTACCAGTGGCTGCTGCCAGTGGCGATAAGTCGTGTCTTACCGG |
| | | GTTGGACTCAAGACGATAGTTACCGGATAAGGCGCAGCGGTCGGGCTGA |
| | | ACGGGGGGTTCGTGCACACAGCCCAGCTTGGAGCGAACGACCTACACCG |
| | | AACTGAGATACCTACAGCGTGAGCTATGAGAAAGCGCCACGCTTCCCGA |
| | | AGGGAGAAAGGCGGACAGGTATCCGGTAAGCGGCAGGGTCGGAACAGG |
| | | AGAGCGCACGAGGGAGCTTCCAGGGGGAAACGCCTGGTATCTTTATAGT |
| | | CCTGTCGGGTTTCGCCACCTCTGACTTGAGCGTCGATTTTTGTGATGCTCG |
| | | TCAGGGGGGCGGAGCCTATGGAAAAACGCCAGCAACGCGGCCTTTTTAC |
| | | GGTTCCTGGCCTTTTGCTGGCCTTTTGCTCACATGTTCTTTCCTGCGTTATC |
| | | CCCTGATTCTGTGGATAACCGTATTACCGCCTTTGAGTGAGCTGATACCG |
| | | CTCGCCGCAGCCGAACGACCGAGCGCAGCGAGTCAGTGAGCGAGGAAGC |
| | | GGAAGAGCGCCCAATACGCAAACCGCCTCTCCCCGCGCGTTGGCCGATTC |
| | | ATTAATG |
| 149 | <#3105 | GTAGAAAAGATCAAAGGATCTTCTTGAGATCCTTTTTTTCTGCGCGTAATC |
| | pAAV_FOXP3. | TGCTGCTTGCAAACAAAAAAACCACCGCTACCAGCGGTGGTTTGTTTGCC |
| | 08_MND.GFPki | GGATCAAGAGCTACCAACTCTTTTTCCGAAGGTAACTGGCTTCAGCAGAG |
| | (1staa)_08_for | CGCAGATACCAAATACTGTCCTTCTAGTGTAGCCGTAGTTAGGCCACCAC |
| | T9> | TTCAAGAACTCTGTAGCACCGCCTACATACCTCGCTCTGCTAATCCTGTTA |
| | | CCAGTGGCTGCTGCCAGTGGCGATAAGTCGTGTCTTACCGGGTTGGACTC |
| | | AAGACGATAGTTACCGGATAAGGCGCAGCGGTCGGGCTGAACGGGGGGT |
| | | TCGTGCACACAGCCCAGCTTGGAGCGAACGACCTACACCGAACTGAGAT |
| | | ACCTACAGCGTGAGCTATGAGAAAGCGCCACGCTTCCCGAAGGGAGAAA |
| | | GGCGGACAGGTATCCGGTAAGCGGCAGGGTCGGAACAGGAGAGCGCACG |
| | | AGGGAGCTTCCAGGGGGAAACGCCTGGTATCTTTATAGTCCTGTCGGGTT |
| | | TCGCCACCTCTGACTTGAGCGTCGATTTTTGTGATGCTCGTCAGGGGGGC |
| | | GGAGCCTATGGAAAAACGCCAGCAACGCGGCCTTTTTACGGTTCCTGGCC |
| | | TTTTGCTGGCCTTTTGCTCACATGTTCTTTCCTGCGTTATCCCCTGATTCTG |
| | | TGGATAACCGTATTACCGCCTTTGAGTGAGCTGATACCGCTCGCCGCAGC |
| | | CGAACGACCGAGCGCAGCGAGTCAGTGAGCGAGGAAGCGGAAGAGCGC |
| | | CCAATACGCAAACCGCCTCTCCCCGCGCGTTGGCCGATTCATTAATGCAG |
| | | CTGCGCGCTCGCTCGCTCACTGAGGCCGCCCGGGCAAAGCCCGGGCGTCG |
| | | GGCGACCTTTGGTCGCCCGGCCTCAGTGAGCGAGCGAGCGCGCAGAGAG |
| | | GGAGTGGCCAACTCCATCACTAGGGGTTCCTTGTAGTTAATGATTAACCC |
| | | GCCATGCTACTTATCTACGTAGCGGCCGCATCTCAGGTAATGTCAGCTCG |
| | | GTCCTTCCAGCTGCTCAAGCTAAAACCCATGTCACTTTGACTCTCCCTCTT |
| | | GCCCACTACATCCAAGCTGCTAGCACTGCTCCTGATCCAGCTTCAGATTA |
| | | AGTCTCAGAATCTACCCACTTCTCGCCTTCTCCACTGCCACCAGCCCATTC |
| | | TGTGCCAGCATCATCACTTGCCAGGACTGTTACAATAGCCTCCTCACTAG |
| | | CCCCACTCACAGCAGCCAGATGAATCTTTTGAGTCCATGCCTAGTCACTG |
| | | GGGCAAAATAGGACTCCGAGGAGAAAGTCCGAGACCAGCTCCGGCAAGA |
| | | TGAGCAAACACAGCCTGTGCAGGGTGCAGGGAGGGCTAGAGGCCTGAGG |
| | | CTTGAAACAGCTCTCAAGTGGAGGGGGAAACAACCATTGCCCTCATAGA |
| | | GGACACATCCACACCAGGGCTGTGCTAGCGTGGGCAGGCAAGCCAGGTG |
| | | CTGGACCTCTGCACGTGGGGCATGTGTGGGTATGTACATGTACCTGTGTT |
| | | CTTGGTGTGTGTGTGTGTGTGTGTGTGTGTGTGTGTCTAGAGCTGGGGTGC |
| | | AACTATGGGGCCCCTCGGGACATGTCCCAGCCAATGCCTGCTTTGACCAG |
| | | AGGAGTGTCCACGTGGCTCAGGTGGTCGAGTATCTCATACCGCCCTAGCA |
| | | CACGTGTGACTCCTTTCCCCTATTGTCTACGCAGCCTGCCCTTGGACAAGG |
| | | ACCCGATGCCCAACCCCAGGCCTGGCAAGCCCTCGGCCCCTTCCTTGGCC |
| | | CTTGGCCCATCCCCACGCGTAGGAACAGAGAAACAGGAGAATATGGGCC |
| | | AAACAGGATATCTGTGGTAAGCAGTTCCTGCCCCGGCTCAGGGCCAAGA |
| | | ACAGTTGGAACAGCAGAATATGGGCCAAACAGGATATCTGTGGTAAGCA |
| | | GTTCCTGCCCCGGCTCAGGGCCAAGAACAGATGGTCCCCAGATGCGGTCC |
| | | CGCCCTCAGCAGTTTCTAGAGAACCATCAGATGTTTCCAGGGTGCCCCAA |
| | | GGACCTGAAATGACCCTGTGCCTTATTTGAACTAACCAATCAGTTCGCTT |
| | | CTCGCTTCTGTTCGCGCGCTTCTGCTCCCCGAGCTCTATATAAGCAGAGCT |
| | | CGTTTAGTGAACCGTCAGATCGTCTACGCAGCCTGCCCTTGGACAAGGAC |
| | | CCGATGCCCAACCCCAGGCCTGTGAGCAAGGGCGAGGAGCTGTTCACCG |
| | | GGGTGGTGCCCATCCTGGTCGAGCTGGACGGCGACGTAAACGGCCACAA |
| | | GTTCAGCGTGTCCGGCGAGGGCGAGGGCGATGCCACCTACGGCAAGCTG |
| | | ACCCTGAAGTTCATCTGCACCACCGGCAAGCTGCCCGTGCCCTGGCCCAC |
| | | CCTCGTGACCACCCTGACCTACGGCGTGCAGTGCTTCAGCCGCTACCCCG |
| | | ACCACATGAAGCAGCACGACTTCTTCAAGTCCGCCATGCCCGAAGGCTAC |
| | | GTCCAGGAGCGCACCATCTTCTTCAAGGACGACGGCAACTACAAGACCC |
| | | GCGCCGAGGTGAAGTTCGAGGGCGACACCCTGGTGAACCGCATCGAGCT |
| | | GAAGGGCATCGACTTCAAGGAGGACGGCAACATCCTGGGGCACAAGCTG |
| | | GAGTACAACTACAACAGCCACAACGTCTATATCATGGCCGACAAGCAGA |
| | | AGAACGGCATCAAGGTGAACTTCAAGATCCGCCACAACATCGAGGACGG |
| | | CAGCGTGCAGCTCGCCGACCACTACCAGCAGAACACCCCCATCGGCGAC |
| | | GGCCCCGTGCTGCTGCCCGACAACCACTACCTGAGCACCCAGTCCGCCCT |
| | | GAGCAAAGACCCCAACGAGAAGCGCGATCACATGGTCCTGCTGGAGTTC |
| | | GTGACCGCCGCCGGGATCACTCTCGGCATGGACGAGCTGTACAAGATGCC |
| | | CAACCCCAGGCCTGGCAAGCCCTCGGCCCCTTCCTTGGCCCTTGGCCCAT |
| | | CTCCTGGTGCATCGCCCAGCTGGAGGGCTGCCCCTAAAGCAAGCGACCTG |
| | | CTGGGGGCCCGGGGCCCGGGTGGCACGTTTCAAGGCCGAGATCTTCGAG |
| | | GCGGGGCCCATGCCTCCTCTTCTTCCTTGAACCCCATGCCACCATCGCAGC |
| | | TGCAGGTGAGGCCCTGGGCCCAGGATGGGGCAGGCAGGGTGGGGTACCT |
| | | GGACCTACAGGTGCCGACCTTTACTGTGGCACTGGGCGGGAGGGGGGCT |
| | | GGCTGGGGCACAGGAAGTGGTTTCTGGGTCCCAGGCAAGTCTGTGACTTA |
| | | TGCAGATGTTGCAGGGCCAAGAAAATCCCCACCTGCCAGGCCTCAGAGA |
| | | TTGGAGGCTCTCCCCGACCTCCCAATCCCTGTCTCAGGAGAGGAGGAGGC |
| | | CGTATTGTAGTCCCATGAGCATAGCTATGTGTCCCCATCCCCATGTGACA |
| | | AGAGAAGAGGACTGGGGCCAAGTAGGTGAGGTGACAGGGCTGAGGCCA |
| | | GCTCTGCAACTTATTAGCTGTTTGATCTTTAAAAAGTTACTCGATCTCCAT |
| | | GAGCCTCAGTTTCCATACGTGTAAAAGGGGGATGATCATAGCATCTACCA |
| | | TGTGGGCTTGCAGTGCAGAGTATTTGAATTAGACACAGAACAGTGAGGAT |
| | | CAGGATGGCCTCTCACCCACCTGCCTTTCTGCCCAGCTGCCCACACTGCCC |
| | | CTAGTCATGGTGGCACCCTCCGGGGCACGGCTGGGCCCCTTGCCCCACTT |
| | | ACAGGCACTCCTCCAGGACAGGCCACATTTCATGCACCAGGTATGGACGG |
| | | TGAATGGATCCTACGTAGATAAGTAGCATGGCGGGTTAATCATTAACTAC |
| | | AAGGAACCCCTAGTGATGGAGTTGGCCACTCCCTCTCTGCGCGCTCGCTC |
| | | GCTCACTGAGGCCGGGCGACCAAAGGTCGCCCGACGCCCGGGCTTTGCCC |
| | | GGGCGGCCTCAGTGAGCGAGCGAGCGCGCCAGCTGGCGTAATAGCGAAG |
| | | AGGCCCGCACCGATCGCCCTTCCCAACAGTTGCGCAGCCTGAATGGCGAA |
| | | TGGCGATTCCGTTGCAATGGCTGGCGGTAATATTGTTCTGGATATTACCA |
| | | GCAAGGCCGATAGTTTGAGTTCTTCTACTCAGGCAAGTGATGTTATTACT |
| | | AATCAAAGAAGTATTGCGACAACGGTTAATTTGCGTGATGGACAGACTCT |
| | | TTTACTCGGTGGCCTCACTGATTATAAAAACACTTCTCAGGATTCTGGCGT |
| | | ACCGTTCCTGTCTAAAATCCCTTTAATCGGCCTCCTGTTTAGCTCCCGCTC |
| | | TGATTCTAACGAGGAAAGCACGTTATACGTGCTCGTCAAAGCAACCATAG |
| | | TACGCGCCCTGTAGCGGCGCATTAAGCGCGGCGGGTGTGGTGGTTACGCG |
| | | CAGCGTGACCGCTACACTTGCCAGCGCCCTAGCGCCCGCTCCTTTCGCTTT |
| | | CTTCCCTTCCTTTCTCGCCACGTTCGCCGGCTTTCCCCGTCAAGCTCTAAA |
| | | TCGGGGGCTCCCTTTAGGGTTCCGATTTAGTGCTTTACGGCACCTCGACCC |
| | | CAAAAAACTTGATTAGGGTGATGGTTCACGTAGTGGGCCATCGCCCTGAT |
| | | AGACGGTTTTTCGCCCTTTGACGTTGGAGTCCACGTTCTTTAATAGTGGAC |
| | | TCTTGTTCCAAACTGGAACAACACTCAACCCTATCTCGGTCTATTCTTTTG |
| | | ATTTATAAGGGATTTTGCCGATTTCGGCCTATTGGTTAAAAAATGAGCTG |
| | | ATTTAACAAAAATTTAACGCGAATTTTAACAAAATATTAACGTTTACAAT |
| | | TTAAATATTTGCTTATACAATCTTCCTGTTTTTGGGGCTTTTCTGATTATCA |
| | | ACCGGGGTACATATGATTGACATGCTAGTTTTACGATTACCGTTCATCGA |
| | | TTCTCTTGTTTGCTCCAGACTCTCAGGCAATGACCTGATAGCCTTTGTAGA |
| | | GACCTCTCAAAAATAGCTACCCTCTCCGGCATGAATTTATCAGCTAGAAC |
| | | GGTTGAATATCATATTGATGGTGATTTGACTGTCTCCGGCCTTTCTCACCC |
| | | GTTTGAATCTTTACCTACACATTACTCAGGCATTGCATTTAAAATATATGA |
| | | GGGTTCTAAAAATTTTTATCCTTGCGTTGAAATAAAGGCTTCTCCCGCAA |
| | | AAGTATTACAGGGTCATAATGTTTTTGGTACAACCGATTTAGCTTTATGCT |
| | | CTGAGGCTTTATTGCTTAATTTTGCTAATTCTTTGCCTTGCCTGTATGATTT |
| | | ATTGGATGTTGGAATCGCCTGATGCGGTATTTTCTCCTTACGCATCTGTGC |
| | | GGTATTTCACACCGCATATGGTGCACTCTCAGTACAATCTGCTCTGATGCC |
| | | GCATAGTTAAGCCAGCCCCGACACCCGCCAACACCCGCTGACGCGCCCTG |
| | | ACGGGCTTGTCTGCTCCCGGCATCCGCTTACAGACAAGCTGTGACCGTCT |
| | | CCGGGAGCTGCATGTGTCAGAGGTTTTCACCGTCATCACCGAAACGCGCG |
| | | AGACGAAAGGGCCTCGTGATACGCCTATTTTTATAGGTTAATGTCATGAT |
| | | AATAATGGTTTCTTAGACGTCAGGTGGCACTTTTCGGGGAAATGTGCGCG |
| | | GAACCCCTATTTGTTTATTTTTCTAAATACATTCAAATATGTATCCGCTCA |
| | | TGAGACAATAACCCTGATAAATGCTTCAATAATATTGAAAAAGGAAGAG |
| | | TATGAGTATTCAACATTTCCGTGTCGCCCTTATTCCCTTTTTTGCGGCATTT |
| | | TGCCTTCCTGTTTTTGCTCACCCAGAAACGCTGGTGAAAGTAAAAGATGC |
| | | TGAAGATCAGTTGGGTGCACGAGTGGGTTACATCGAACTGGATCTCAACA |
| | | GCGGTAAGATCCTTGAGAGTTTTCGCCCCGAAGAACGTTTTCCAATGATG |
| | | AGCACTTTTAAAGTTCTGCTATGTGGCGCGGTATTATCCCGTATTGACGCC |
| | | GGGCAAGAGCAACTCGGTCGCCGCATACACTATTCTCAGAATGACTTGGT |
| | | TGAGTACTCACCAGTCACAGAAAAGCATCTTACGGATGGCATGACAGTA |
| | | AGAGAATTATGCAGTGCTGCCATAACCATGAGTGATAACACTGCGGCCA |
| | | ACTTACTTCTGACAACGATCGGAGGACCGAAGGAGCTAACCGCTTTTTTG |
| | | CACAACATGGGGGATCATGTAACTCGCCTTGATCGTTGGGAACCGGAGCT |
| | | GAATGAAGCCATACCAAACGACGAGCGTGACACCACGATGCCTGTAGCA |
| | | ATGGCAACAACGTTGCGCAAACTATTAACTGGCGAACTACTTACTCTAGC |
| | | TTCCCGGCAACAATTAATAGACTGGATGGAGGCGGATAAAGTTGCAGGA |
| | | CCACTTCTGCGCTCGGCCCTTCCGGCTGGCTGGTTTATTGCTGATAAATCT |
| | | GGAGCCGGTGAGCGTGGGTCTCGCGGTATCATTGCAGCACTGGGGCCAG |
| | | ATGGTAAGCCCTCCCGTATCGTAGTTATCTACACGACGGGGAGTCAGGCA |
| | | ACTATGGATGAACGAAATAGACAGATCGCTGAGATAGGTGCCTCACTGA |
| | | TTAAGCATTGGTAACTGTCAGACCAAGTTTACTCATATATACTTTAGATTG |
| | | ATTTAAAACTTCATTTTTAATTTAAAAGGATCTAGGTGAAGATCCTTTTTG |
| | | ATAATCTCATGACCAAAATCCCTTAACGTGAGTTTTCGTTCCACTGAGCGT |
| | | CAGACCCC |
| 150 | <#3066 | GTAGAAAAGATCAAAGGATCTTCTTGAGATCCTTTTTTTCTGCGCGTAATC |
| | pAAV_FOXP3. | TGCTGCTTGCAAACAAAAAAACCACCGCTACCAGCGGTGGTTTGTTTGCC |
| | 06_MND.FOXP | GGATCAAGAGCTACCAACTCTTTTTCCGAAGGTAACTGGCTTCAGCAGAG |
| | 3geneartCDS.P2 | CGCAGATACCAAATACTGTCCTTCTAGTGTAGCCGTAGTTAGGCCACCAC |
| | A.LNGFR.pA_0 | TTCAAGAACTCTGTAGCACCGCCTACATACCTCGCTCTGCTAATCCTGTTA |
| | 6_for T9> | CCAGTGGCTGCTGCCAGTGGCGATAAGTCGTGTCTTACCGGGTTGGACTC |
| | | AAGACGATAGTTACCGGATAAGGCGCAGCGGTCGGGCTGAACGGGGGGT |
| | | TCGTGCACACAGCCCAGCTTGGAGCGAACGACCTACACCGAACTGAGAT |
| | | ACCTACAGCGTGAGCTATGAGAAAGCGCCACGCTTCCCGAAGGGAGAAA |
| | | GGCGGACAGGTATCCGGTAAGCGGCAGGGTCGGAACAGGAGAGCGCACG |
| | | AGGGAGCTTCCAGGGGGAAACGCCTGGTATCTTTATAGTCCTGTCGGGTT |
| | | TCGCCACCTCTGACTTGAGCGTCGATTTTTGTGATGCTCGTCAGGGGGGC |
| | | GGAGCCTATGGAAAAACGCCAGCAACGCGGCCTTTTTACGGTTCCTGGCC |
| | | TTTTGCTGGCCTTTTGCTCACATGTTCTTTCCTGCGTTATCCCCTGATTCTG |
| | | TGGATAACCGTATTACCGCCTTTGAGTGAGCTGATACCGCTCGCCGCAGC |
| | | CGAACGACCGAGCGCAGCGAGTCAGTGAGCGAGGAAGCGGAAGAGCGC |
| | | CCAATACGCAAACCGCCTCTCCCCGCGCGTTGGCCGATTCATTAATGCAG |
| | | CTGCGCGCTCGCTCGCTCACTGAGGCCGCCCGGGCAAAGCCCGGGCGTCG |
| | | GGCGACCTTTGGTCGCCCGGCCTCAGTGAGCGAGCGAGCGCGCAGAGAG |
| | | GGAGTGGCCAACTCCATCACTAGGGGTTCCTTGTAGTTAATGATTAACCC |
| | | GCCATGCTACTTATCTACGTAGCGGCCGCATCACTTGCCAGGACTGTTAC |
| | | AATAGCCTCCTCACTAGCCCCACTCACAGCAGCCAGATGAATCTTTTGAG |
| | | TCCATGCCTAGTCACTGGGGCAAAATAGGACTCCGAGGAGAAAGTCCGA |
| | | GACCAGCTCCGGCAAGATGAGCAAACACAGCCTGTGCAGGGTGCAGGGA |
| | | GGGCTAGAGGCCTGAGGCTTGAAACAGCTCTCAAGTGGAGGGGGAAACA |
| | | ACCATTGCCCTCATAGAGGACACATCCACACCAGGGCTGTGCTAGCGTGG |
| | | GCAGGCAAGCCAGGTGCTGGACCTCTGCACGTGGGGCATGTGTGGGTAT |
| | | GTACATGTACCTGTGTTCTTGGTGTGTGTGTGTGTGTGTGTGTGTGTGTGT |
| | | GTCTAGAGCTGGGGTGCAACTATGGGGCCCCTCGGGACATGTCCCAGCCA |
| | | ATGCCTGCTTTGACCAGAGGAGTGTCCACGTGGCTCAGGTGGTCGAGTAT |
| | | CTCATACCGCCCTAGCACACGTGTGACTCCTTTCCCCTATTGTCTACGCAG |
| | | CCTGCCCTTGGACAAGGACCCGATGCCCAACCCCAGGCCTGGCAAGCCCT |
| | | CGGCCCCTTCCTTGGCCCTTGGCCCATCCCCACGCGTAGGAACAGAGAAA |
| | | CAGGAGAATATGGGCCAAACAGGATATCTGTGGTAAGCAGTTCCTGCCCC |
| | | GGCTCAGGGCCAAGAACAGTTGGAACAGCAGAATATGGGCCAAACAGGA |
| | | TATCTGTGGTAAGCAGTTCCTGCCCCGGCTCAGGGCCAAGAACAGATGGT |
| | | CCCCAGATGCGGTCCCGCCCTCAGCAGTTTCTAGAGAACCATCAGATGTT |
| | | TCCAGGGTGCCCCAAGGACCTGAAATGACCCTGTGCCTTATTTGAACTAA |
| | | CCAATCAGTTCGCTTCTCGCTTCTGTTCGCGCGCTTCTGCTCCCCGAGCTC |
| | | TATATAAGCAGAGCTCGTTTAGTGAACCGTCAGATCGCCTGGAGACGCCA |
| | | TCCACGCTGTTTTGACTTCCATAGAAGGATCTCGAGGCCACCATGCCTAA |
| | | TCCTCGGCCTGGAAAGCCTAGCGCTCCTTCTCTTGCTCTGGGACCTTCTCC |
| | | TGGCGCCTCTCCATCTTGGAGAGCCGCTCCTAAAGCCAGCGATCTGCTGG |
| | | GAGCTAGAGGACCTGGCGGCACATTTCAGGGCAGAGATCTTAGAGGCGG |
| | | AGCCCACGCTAGCTCCTCCAGCCTTAATCCTATGCCTCCTAGCCAGCTCCA |
| | | GCTGCCTACACTGCCTCTGGTTATGGTGGCTCCTAGCGGAGCTAGACTGG |
| | | GCCCTCTGCCTCATCTGCAAGCTCTGCTGCAGGACAGACCCCACTTCATG |
| | | CACCAGCTGAGCACCGTGGATGCCCACGCAAGAACACCTGTGCTGCAGG |
| | | TTCACCCTCTGGAATCCCCAGCCATGATCAGCCTGACACCTCCAACAACA |
| | | GCCACCGGCGTGTTCAGCCTGAAAGCCAGACCTGGACTGCCTCCTGGCAT |
| | | CAATGTGGCCAGCCTGGAATGGGTGTCCAGAGAACCTGCTCTGCTGTGCA |
| | | CATTCCCCAATCCAAGCGCTCCCAGAAAGGACAGCACACTGTCTGCCGTG |
| | | CCTCAGAGCAGCTATCCCCTGCTTGCTAACGGCGTGTGCAAGTGGCCTGG |
| | | ATGCGAGAAGGTGTTCGAGGAACCCGAGGACTTCCTGAAGCACTGCCAG |
| | | GCCGATCATCTGCTGGACGAGAAAGGCAGAGCCCAGTGTCTGCTCCAGC |
| | | GCGAGATGGTGCAGTCTCTGGAACAGCAGCTGGTCCTGGAAAAAGAAAA |
| | | GCTGAGCGCCATGCAGGCCCACCTGGCCGGAAAAATGGCCCTGACAAAG |
| | | GCCAGCAGCGTGGCCTCTTCTGATAAGGGCAGCTGCTGCATTGTGGCCGC |
| | | TGGATCTCAGGGACCTGTGGTTCCTGCTTGGAGCGGACCTAGAGAGGCCC |
| | | CTGATTCTCTGTTTGCCGTGCGGAGACACCTGTGGGGCTCTCACGGCAAC |
| | | TCTACTTTCCCCGAGTTCCTGCACAACATGGACTACTTCAAGTTCCACAAC |
| | | ATGCGGCCTCCATTCACCTACGCCACACTGATCAGATGGGCCATTCTGGA |
| | | AGCCCCTGAGAAGCAGAGAACCCTGAACGAGATCTACCACTGGTTTACCC |
| | | GGATGTTCGCCTTCTTCCGGAATCACCCTGCCACCTGGAAGAACGCCATC |
| | | CGGCACAATCTGAGCCTGCACAAGTGCTTCGTGCGCGTGGAATCTGAGAA |
| | | AGGCGCCGTGTGGACAGTGGACGAGCTGGAATTCAGAAAGAAGAGAAGC |
| | | CAGCGGCCTAGCCGGTGCAGCAATCCTACACCTGGACCTGGAAGCGGAG |
| | | CGACTAACTTCAGCCTGCTGAAGCAGGCCGGAGATGTGGAGGAAAACCC |
| | | TGGACCGATGGGGGCAGGTGCCACCGGACGAGCCATGGACGGGCCGCGC |
| | | CTGCTGCTGTTGCTGCTTCTGGGGGTGTCCCTTGGAGGTGCCAAGGAGGC |
| | | ATGCCCCACAGGCCTGTACACACACAGCGGTGAGTGCTGCAAAGCCTGC |
| | | AACCTGGGCGAGGGTGTGGCCCAGCCTTGTGGAGCCAACCAGACCGTGT |
| | | GTGAGCCCTGCCTGGACAGCGTGACGTTCTCCGACGTGGTGAGCGCGACC |
| | | GAGCCGTGCAAGCCGTGCACCGAGTGCGTGGGGCTCCAGAGCATGTCGG |
| | | CGCCGTGCGTGGAGGCCGACGACGCCGTGTGCCGCTGCGCCTACGGCTAC |
| | | TACCAGGATGAGACGACTGGGCGCTGCGAGGCGTGCCGCGTGTGCGAGG |
| | | CGGGCTCGGGCCTCGTGTTCTCCTGCCAGGACAAGCAGAACACCGTGTGC |
| | | GAGGAGTGCCCCGACGGCACGTATTCCGACGAGGCCAACCACGTGGACC |
| | | CGTGCCTGCCCTGCACCGTGTGCGAGGACACCGAGCGCCAGCTCCGCGAG |
| | | TGCACACGCTGGGCCGACGCCGAGTGCGAGGAGATCCCTGGCCGTTGGA |
| | | TTACACGGTCCACACCCCCAGAGGGCTCGGACAGCACAGCCCCCAGCAC |
| | | CCAGGAGCCTGAGGCACCTCCAGAACAAGACCTCATAGCCAGCACGGTG |
| | | GCAGGTGTGGTGACCACAGTGATGGGCAGCTCCCAGCCCGTGGTGACCC |
| | | GAGGCACCACCGACAACCTCATCCCTGTCTATTGCTCCATCCTGGCTGCT |
| | | GTGGTTGTGGGTCTTGTGGCCTACATAGCCTTCAAGAGGTGAAAGCTTGT |
| | | CGACTGCTTTATTTGTGAAATTTGTGATGCTATTGCTTTATTTGTAACCAT |
| | | TATAAGCTGCAATAAACAAGTTAACAACAACAATTGCATTCATTTTATGT |
| | | TTCAGGTTCAGGGGGAGATGTGGGAGGTTTTTTAAAGCACTAGTGCCTCG |
| | | CCCAGCTGGAGGGCTGCACCCAAAGCCTCAGACCTGCTGGGGGCCCGGG |
| | | GCCCAGGGGGAACCTTCCAGGGCCGAGATCTTCGAGGCGGGGCCCATGC |
| | | CTCCTCTTCTTCCTTGAACCCCATGCCACCATCGCAGCTGCAGGTGAGGCC |
| | | CTGGGCCCAGGATGGGGCAGGCAGGGTGGGGTACCTGGACCTACAGGTG |
| | | CCGACCTTTACTGTGGCACTGGGCGGGAGGGGGGCTGGCTGGGGCACAG |
| | | GAAGTGGTTTCTGGGTCCCAGGCAAGTCTGTGACTTATGCAGATGTTGCA |
| | | GGGCCAAGAAAATCCCCACCTGCCAGGCCTCAGAGATTGGAGGCTCTCCC |
| | | CGACCTCCCAATCCCTGTCTCAGGAGAGGAGGAGGCCGTATTGTAGTCCC |
| | | ATGAGCATAGCTATGTGTCCCCATCCCCATGTGACAAGAGAAGAGGACTG |
| | | GGGCCAAGTAGGTGAGGTGACAGGGCTGAGGCCAGCTCTGCAACTTATT |
| | | AGCTGTTTGATCTTTAAAAAGTTACTCGATCTCCATGAGCCTCAGTTTCCA |
| | | TACGTGTAAAAGGGGGATGATCATAGCATCTACCATGTGGGCTTGCAGGA |
| | | TCCTACGTAGATAAGTAGCATGGCGGGTTAATCATTAACTACAAGGAACC |
| | | CCTAGTGATGGAGTTGGCCACTCCCTCTCTGCGCGCTCGCTCGCTCACTGA |
| | | GGCCGGGCGACCAAAGGTCGCCCGACGCCCGGGCTTTGCCCGGGCGGCC |
| | | TCAGTGAGCGAGCGAGCGCGCCAGCTGGCGTAATAGCGAAGAGGCCCGC |
| | | ACCGATCGCCCTTCCCAACAGTTGCGCAGCCTGAATGGCGAATGGCGATT |
| | | CCGTTGCAATGGCTGGCGGTAATATTGTTCTGGATATTACCAGCAAGGCC |
| | | GATAGTTTGAGTTCTTCTACTCAGGCAAGTGATGTTATTACTAATCAAAG |
| | | AAGTATTGCGACAACGGTTAATTTGCGTGATGGACAGACTCTTTTACTCG |
| | | GTGGCCTCACTGATTATAAAAACACTTCTCAGGATTCTGGCGTACCGTTC |
| | | CTGTCTAAAATCCCTTTAATCGGCCTCCTGTTTAGCTCCCGCTCTGATTCT |
| | | AACGAGGAAAGCACGTTATACGTGCTCGTCAAAGCAACCATAGTACGCG |
| | | CCCTGTAGCGGCGCATTAAGCGCGGCGGGTGTGGTGGTTACGCGCAGCGT |
| | | GACCGCTACACTTGCCAGCGCCCTAGCGCCCGCTCCTTTCGCTTTCTTCCC |
| | | TTCCTTTCTCGCCACGTTCGCCGGCTTTCCCCGTCAAGCTCTAAATCGGGG |
| | | GCTCCCTTTAGGGTTCCGATTTAGTGCTTTACGGCACCTCGACCCCAAAA |
| | | AACTTGATTAGGGTGATGGTTCACGTAGTGGGCCATCGCCCTGATAGACG |
| | | GTTTTTCGCCCTTTGACGTTGGAGTCCACGTTCTTTAATAGTGGACTCTTG |
| | | TTCCAAACTGGAACAACACTCAACCCTATCTCGGTCTATTCTTTTGATTTA |
| | | TAAGGGATTTTGCCGATTTCGGCCTATTGGTTAAAAAATGAGCTGATTTA |
| | | ACAAAAATTTAACGCGAATTTTAACAAAATATTAACGTTTACAATTTAAA |
| | | TATTTGCTTATACAATCTTCCTGTTTTTGGGGCTTTTCTGATTATCAACCGG |
| | | GGTACATATGATTGACATGCTAGTTTTACGATTACCGTTCATCGATTCTCT |
| | | TGTTTGCTCCAGACTCTCAGGCAATGACCTGATAGCCTTTGTAGAGACCT |
| | | CTCAAAAATAGCTACCCTCTCCGGCATGAATTTATCAGCTAGAACGGTTG |
| | | AATATCATATTGATGGTGATTTGACTGTCTCCGGCCTTTCTCACCCGTTTG |
| | | AATCTTTACCTACACATTACTCAGGCATTGCATTTAAAATATATGAGGGTT |
| | | CTAAAAATTTTTATCCTTGCGTTGAAATAAAGGCTTCTCCCGCAAAAGTA |
| | | TTACAGGGTCATAATGTTTTTGGTACAACCGATTTAGCTTTATGCTCTGAG |
| | | GCTTTATTGCTTAATTTTGCTAATTCTTTGCCTTGCCTGTATGATTTATTGG |
| | | ATGTTGGAATCGCCTGATGCGGTATTTTCTCCTTACGCATCTGTGCGGTAT |
| | | TTCACACCGCATATGGTGCACTCTCAGTACAATCTGCTCTGATGCCGCAT |
| | | AGTTAAGCCAGCCCCGACACCCGCCAACACCCGCTGACGCGCCCTGACG |
| | | GGCTTGTCTGCTCCCGGCATCCGCTTACAGACAAGCTGTGACCGTCTCCG |
| | | GGAGCTGCATGTGTCAGAGGTTTTCACCGTCATCACCGAAACGCGCGAGA |
| | | CGAAAGGGCCTCGTGATACGCCTATTTTTATAGGTTAATGTCATGATAAT |
| | | AATGGTTTCTTAGACGTCAGGTGGCACTTTTCGGGGAAATGTGCGCGGAA |
| | | CCCCTATTTGTTTATTTTTCTAAATACATTCAAATATGTATCCGCTCATGA |
| | | GACAATAACCCTGATAAATGCTTCAATAATATTGAAAAAGGAAGAGTAT |
| | | GAGTATTCAACATTTCCGTGTCGCCCTTATTCCCTTTTTTGCGGCATTTTGC |
| | | CTTCCTGTTTTTGCTCACCCAGAAACGCTGGTGAAAGTAAAAGATGCTGA |
| | | AGATCAGTTGGGTGCACGAGTGGGTTACATCGAACTGGATCTCAACAGCG |
| | | GTAAGATCCTTGAGAGTTTTCGCCCCGAAGAACGTTTTCCAATGATGAGC |
| | | ACTTTTAAAGTTCTGCTATGTGGCGCGGTATTATCCCGTATTGACGCCGGG |
| | | CAAGAGCAACTCGGTCGCCGCATACACTATTCTCAGAATGACTTGGTTGA |
| | | GTACTCACCAGTCACAGAAAAGCATCTTACGGATGGCATGACAGTAAGA |
| | | GAATTATGCAGTGCTGCCATAACCATGAGTGATAACACTGCGGCCAACTT |
| | | ACTTCTGACAACGATCGGAGGACCGAAGGAGCTAACCGCTTTTTTGCACA |
| | | ACATGGGGGATCATGTAACTCGCCTTGATCGTTGGGAACCGGAGCTGAAT |
| | | GAAGCCATACCAAACGACGAGCGTGACACCACGATGCCTGTAGCAATGG |
| | | CAACAACGTTGCGCAAACTATTAACTGGCGAACTACTTACTCTAGCTTCC |
| | | CGGCAACAATTAATAGACTGGATGGAGGCGGATAAAGTTGCAGGACCAC |
| | | TTCTGCGCTCGGCCCTTCCGGCTGGCTGGTTTATTGCTGATAAATCTGGAG |
| | | CCGGTGAGCGTGGGTCTCGCGGTATCATTGCAGCACTGGGGCCAGATGGT |
| | | AAGCCCTCCCGTATCGTAGTTATCTACACGACGGGGAGTCAGGCAACTAT |
| | | GGATGAACGAAATAGACAGATCGCTGAGATAGGTGCCTCACTGATTAAG |
| | | CATTGGTAACTGTCAGACCAAGTTTACTCATATATACTTTAGATTGATTTA |
| | | AAACTTCATTTTTAATTTAAAAGGATCTAGGTGAAGATCCTTTTTGATAAT |
| | | CTCATGACCAAAATCCCTTAACGTGAGTTTTCGTTCCACTGAGCGTCAGA |
| | | CCCC |
| 151 | <#3080 | GTAGAAAAGATCAAAGGATCTTCTTGAGATCCTTTTTTTCTGCGCGTAATC |
| | pAAV_FOXP3. | TGCTGCTTGCAAACAAAAAAACCACCGCTACCAGCGGTGGTTTGTTTGCC |
| | 06_MND.LNGF | GGATCAAGAGCTACCAACTCTTTTTCCGAAGGTAACTGGCTTCAGCAGAG |
| | R-P2A-Ki_0.6 | CGCAGATACCAAATACTGTCCTTCTAGTGTAGCCGTAGTTAGGCCACCAC |
| | for KI> | TTCAAGAACTCTGTAGCACCGCCTACATACCTCGCTCTGCTAATCCTGTTA |
| | | CCAGTGGCTGCTGCCAGTGGCGATAAGTCGTGTCTTACCGGGTTGGACTC |
| | | AAGACGATAGTTACCGGATAAGGCGCAGCGGTCGGGCTGAACGGGGGGT |
| | | TCGTGCACACAGCCCAGCTTGGAGCGAACGACCTACACCGAACTGAGAT |
| | | ACCTACAGCGTGAGCTATGAGAAAGCGCCACGCTTCCCGAAGGGAGAAA |
| | | GGCGGACAGGTATCCGGTAAGCGGCAGGGTCGGAACAGGAGAGCGCACG |
| | | AGGGAGCTTCCAGGGGGAAACGCCTGGTATCTTTATAGTCCTGTCGGGTT |
| | | TCGCCACCTCTGACTTGAGCGTCGATTTTTGTGATGCTCGTCAGGGGGGC |
| | | GGAGCCTATGGAAAAACGCCAGCAACGCGGCCTTTTTACGGTTCCTGGCC |
| | | TTTTGCTGGCCTTTTGCTCACATGTTCTTTCCTGCGTTATCCCCTGATTCTG |
| | | TGGATAACCGTATTACCGCCTTTGAGTGAGCTGATACCGCTCGCCGCAGC |
| | | CGAACGACCGAGCGCAGCGAGTCAGTGAGCGAGGAAGCGGAAGAGCGC |
| | | CCAATACGCAAACCGCCTCTCCCCGCGCGTTGGCCGATTCATTAATGCAG |
| | | CTGCGCGCTCGCTCGCTCACTGAGGCCGCCCGGGCAAAGCCCGGGCGTCG |
| | | GGCGACCTTTGGTCGCCCGGCCTCAGTGAGCGAGCGAGCGCGCAGAGAG |
| | | GGAGTGGCCAACTCCATCACTAGGGGTTCCTTGTAGTTAATGATTAACCC |
| | | GCCATGCTACTTATCTACGTAGCGGCCGCATCACTTGCCAGGACTGTTAC |
| | | AATAGCCTCCTCACTAGCCCCACTCACAGCAGCCAGATGAATCTTTTGAG |
| | | TCCATGCCTAGTCACTGGGGCAAAATAGGACTCCGAGGAGAAAGTCCGA |
| | | GACCAGCTCCGGCAAGATGAGCAAACACAGCCTGTGCAGGGTGCAGGGA |
| | | GGGCTAGAGGCCTGAGGCTTGAAACAGCTCTCAAGTGGAGGGGGAAACA |
| | | ACCATTGCCCTCATAGAGGACACATCCACACCAGGGCTGTGCTAGCGTGG |
| | | GCAGGCAAGCCAGGTGCTGGACCTCTGCACGTGGGGCATGTGTGGGTAT |
| | | GTACATGTACCTGTGTTCTTGGTGTGTGTGTGTGTGTGTGTGTGTGTGTGT |
| | | GTCTAGAGCTGGGGTGCAACTATGGGGCCCCTCGGGACATGTCCCAGCCA |
| | | ATGCCTGCTTTGACCAGAGGAGTGTCCACGTGGCTCAGGTGGTCGAGTAT |
| | | CTCATACCGCCCTAGCACACGTGTGACTCCTTTCCCCTATTGTCTACGCAG |
| | | CCTGCCCTTGGACAAGGACCCGATGCCCAACCCCAGGCCTGGCAAGCCCT |
| | | CGGCCCCTTCCTTGGCCCTTGGCCCATCCCCACGCGTAGGAACAGAGAAA |
| | | CAGGAGAATATGGGCCAAACAGGATATCTGTGGTAAGCAGTTCCTGCCCC |
| | | GGCTCAGGGCCAAGAACAGTTGGAACAGCAGAATATGGGCCAAACAGGA |
| | | TATCTGTGGTAAGCAGTTCCTGCCCCGGCTCAGGGCCAAGAACAGATGGT |
| | | CCCCAGATGCGGTCCCGCCCTCAGCAGTTTCTAGAGAACCATCAGATGTT |
| | | TCCAGGGTGCCCCAAGGACCTGAAATGACCCTGTGCCTTATTTGAACTAA |
| | | CCAATCAGTTCGCTTCTCGCTTCTGTTCGCGCGCTTCTGCTCCCCGAGCTC |
| | | TATATAAGCAGAGCTCGTTTAGTGAACCGTCAGATCGCCTGGAGACGCCA |
| | | TCCACGCTGTTTTGACTTCCATAGAAGGATCTCGAGGCCACCATGGGGGC |
| | | AGGTGCCACCGGACGAGCCATGGACGGGCCGCGCCTGCTGCTGTTGCTGC |
| | | TTCTGGGGGTGTCCCTTGGAGGTGCCAAGGAGGCATGCCCCACAGGCCTG |
| | | TACACACACAGCGGTGAGTGCTGCAAAGCCTGCAACCTGGGCGAGGGTG |
| | | TGGCCCAGCCTTGTGGAGCCAACCAGACCGTGTGTGAGCCCTGCCTGGAC |
| | | AGCGTGACGTTCTCCGACGTGGTGAGCGCGACCGAGCCGTGCAAGCCGT |
| | | GCACCGAGTGCGTGGGGCTCCAGAGCATGTCGGCGCCGTGCGTGGAGGC |
| | | CGACGACGCCGTGTGCCGCTGCGCCTACGGCTACTACCAGGATGAGACG |
| | | ACTGGGCGCTGCGAGGCGTGCCGCGTGTGCGAGGCGGGCTCGGGCCTCG |
| | | TGTTCTCCTGCCAGGACAAGCAGAACACCGTGTGCGAGGAGTGCCCCGAC |
| | | GGCACGTATTCCGACGAGGCCAACCACGTGGACCCGTGCCTGCCCTGCAC |
| | | CGTGTGCGAGGACACCGAGCGCCAGCTCCGCGAGTGCACACGCTGGGCC |
| | | GACGCCGAGTGCGAGGAGATCCCTGGCCGTTGGATTACACGGTCCACACC |
| | | CCCAGAGGGCTCGGACAGCACAGCCCCCAGCACCCAGGAGCCTGAGGCA |
| | | CCTCCAGAACAAGACCTCATAGCCAGCACGGTGGCAGGTGTGGTGACCA |
| | | CAGTGATGGGCAGCTCCCAGCCCGTGGTGACCCGAGGCACCACCGACAA |
| | | CCTCATCCCTGTCTATTGCTCCATCCTGGCTGCTGTGGTTGTGGGTCTTGT |
| | | GGCCTACATAGCCTTCAAGAGGGGAAGCGGAGCGACTAACTTCAGCCTG |
| | | CTGAAGCAGGCCGGAGATGTGGAGGAAAACCCTGGACCGATGCCCAACC |
| | | CCAGGCCTGGCAAGCCCTCGGCCCCTTCCTTGGCCCTTGGCCCATCTCCTG |
| | | GTGCATCGCCCAGCTGGAGGGCTGCCCCTAAAGCAAGCGACCTGCTGGG |
| | | GGCCCGGGGCCCGGGTGGCACGTTCCAGGGCCGAGATCTTCGAGGCGGG |
| | | GCCCATGCCTCCTCTTCTTCCTTGAACCCCATGCCACCATCGCAGCTGCAG |
| | | GTGAGGCCCTGGGCCCAGGATGGGGCAGGCAGGGTGGGGTACCTGGACC |
| | | TACAGGTGCCGACCTTTACTGTGGCACTGGGCGGGAGGGGGGCTGGCTG |
| | | GGGCACAGGAAGTGGTTTCTGGGTCCCAGGCAAGTCTGTGACTTATGCAG |
| | | ATGTTGCAGGGCCAAGAAAATCCCCACCTGCCAGGCCTCAGAGATTGGA |
| | | GGCTCTCCCCGACCTCCCAATCCCTGTCTCAGGAGAGGAGGAGGCCGTAT |
| | | TGTAGTCCCATGAGCATAGCTATGTGTCCCCATCCCCATGTGACAAGAGA |
| | | AGAGGACTGGGGCCAAGTAGGTGAGGTGACAGGGCTGAGGCCAGCTCTG |
| | | CAACTTATTAGCTGTTTGATCTTTAAAAAGTTACTCGATCTCCATGAGCCT |
| | | CAGTTTCCATACGTGTAAAAGGGGGATGATCATAGCATCTACCATGTGGG |
| | | CTTGCAGGATCCTACGTAGATAAGTAGCATGGCGGGTTAATCATTAACTA |
| | | CAAGGAACCCCTAGTGATGGAGTTGGCCACTCCCTCTCTGCGCGCTCGCT |
| | | CGCTCACTGAGGCCGGGCGACCAAAGGTCGCCCGACGCCCGGGCTTTGCC |
| | | CGGGCGGCCTCAGTGAGCGAGCGAGCGCGCCAGCTGGCGTAATAGCGAA |
| | | GAGGCCCGCACCGATCGCCCTTCCCAACAGTTGCGCAGCCTGAATGGCGA |
| | | ATGGCGATTCCGTTGCAATGGCTGGCGGTAATATTGTTCTGGATATTACC |
| | | AGCAAGGCCGATAGTTTGAGTTCTTCTACTCAGGCAAGTGATGTTATTAC |
| | | TAATCAAAGAAGTATTGCGACAACGGTTAATTTGCGTGATGGACAGACTC |
| | | TTTTACTCGGTGGCCTCACTGATTATAAAAACACTTCTCAGGATTCTGGCG |
| | | TACCGTTCCTGTCTAAAATCCCTTTAATCGGCCTCCTGTTTAGCTCCCGCT |
| | | CTGATTCTAACGAGGAAAGCACGTTATACGTGCTCGTCAAAGCAACCATA |
| | | GTACGCGCCCTGTAGCGGCGCATTAAGCGCGGCGGGTGTGGTGGTTACGC |
| | | GCAGCGTGACCGCTACACTTGCCAGCGCCCTAGCGCCCGCTCCTTTCGCT |
| | | TTCTTCCCTTCCTTTCTCGCCACGTTCGCCGGCTTTCCCCGTCAAGCTCTAA |
| | | ATCGGGGGCTCCCTTTAGGGTTCCGATTTAGTGCTTTACGGCACCTCGACC |
| | | CCAAAAAACTTGATTAGGGTGATGGTTCACGTAGTGGGCCATCGCCCTGA |
| | | TAGACGGTTTTTCGCCCTTTGACGTTGGAGTCCACGTTCTTTAATAGTGGA |
| | | CTCTTGTTCCAAACTGGAACAACACTCAACCCTATCTCGGTCTATTCTTTT |
| | | GATTTATAAGGGATTTTGCCGATTTCGGCCTATTGGTTAAAAAATGAGCT |
| | | GATTTAACAAAAATTTAACGCGAATTTTAACAAAATATTAACGTTTACAA |
| | | TTTAAATATTTGCTTATACAATCTTCCTGTTTTTGGGGCTTTTCTGATTATC |
| | | AACCGGGGTACATATGATTGACATGCTAGTTTTACGATTACCGTTCATCG |
| | | ATTCTCTTGTTTGCTCCAGACTCTCAGGCAATGACCTGATAGCCTTTGTAG |
| | | AGACCTCTCAAAAATAGCTACCCTCTCCGGCATGAATTTATCAGCTAGAA |
| | | CGGTTGAATATCATATTGATGGTGATTTGACTGTCTCCGGCCTTTCTCACC |
| | | CGTTTGAATCTTTACCTACACATTACTCAGGCATTGCATTTAAAATATATG |
| | | AGGGTTCTAAAAATTTTTATCCTTGCGTTGAAATAAAGGCTTCTCCCGCA |
| | | AAAGTATTACAGGGTCATAATGTTTTTGGTACAACCGATTTAGCTTTATGC |
| | | TCTGAGGCTTTATTGCTTAATTTTGCTAATTCTTTGCCTTGCCTGTATGATT |
| | | TATTGGATGTTGGAATCGCCTGATGCGGTATTTTCTCCTTACGCATCTGTG |
| | | CGGTATTTCACACCGCATATGGTGCACTCTCAGTACAATCTGCTCTGATGC |
| | | CGCATAGTTAAGCCAGCCCCGACACCCGCCAACACCCGCTGACGCGCCCT |
| | | GACGGGCTTGTCTGCTCCCGGCATCCGCTTACAGACAAGCTGTGACCGTC |
| | | TCCGGGAGCTGCATGTGTCAGAGGTTTTCACCGTCATCACCGAAACGCGC |
| | | GAGACGAAAGGGCCTCGTGATACGCCTATTTTTATAGGTTAATGTCATGA |
| | | TAATAATGGTTTCTTAGACGTCAGGTGGCACTTTTCGGGGAAATGTGCGC |
| | | GGAACCCCTATTTGTTTATTTTTCTAAATACATTCAAATATGTATCCGCTC |
| | | ATGAGACAATAACCCTGATAAATGCTTCAATAATATTGAAAAAGGAAGA |
| | | GTATGAGTATTCAACATTTCCGTGTCGCCCTTATTCCCTTTTTTGCGGCAT |
| | | TTTGCCTTCCTGTTTTTGCTCACCCAGAAACGCTGGTGAAAGTAAAAGAT |
| | | GCTGAAGATCAGTTGGGTGCACGAGTGGGTTACATCGAACTGGATCTCAA |
| | | CAGCGGTAAGATCCTTGAGAGTTTTCGCCCCGAAGAACGTTTTCCAATGA |
| | | TGAGCACTTTTAAAGTTCTGCTATGTGGCGCGGTATTATCCCGTATTGACG |
| | | CCGGGCAAGAGCAACTCGGTCGCCGCATACACTATTCTCAGAATGACTTG |
| | | GTTGAGTACTCACCAGTCACAGAAAAGCATCTTACGGATGGCATGACAGT |
| | | AAGAGAATTATGCAGTGCTGCCATAACCATGAGTGATAACACTGCGGCC |
| | | AACTTACTTCTGACAACGATCGGAGGACCGAAGGAGCTAACCGCTTTTTT |
| | | GCACAACATGGGGGATCATGTAACTCGCCTTGATCGTTGGGAACCGGAGC |
| | | TGAATGAAGCCATACCAAACGACGAGCGTGACACCACGATGCCTGTAGC |
| | | AATGGCAACAACGTTGCGCAAACTATTAACTGGCGAACTACTTACTCTAG |
| | | CTTCCCGGCAACAATTAATAGACTGGATGGAGGCGGATAAAGTTGCAGG |
| | | ACCACTTCTGCGCTCGGCCCTTCCGGCTGGCTGGTTTATTGCTGATAAATC |
| | | TGGAGCCGGTGAGCGTGGGTCTCGCGGTATCATTGCAGCACTGGGGCCAG |
| | | ATGGTAAGCCCTCCCGTATCGTAGTTATCTACACGACGGGGAGTCAGGCA |
| | | ACTATGGATGAACGAAATAGACAGATCGCTGAGATAGGTGCCTCACTGA |
| | | TTAAGCATTGGTAACTGTCAGACCAAGTTTACTCATATATACTTTAGATTG |
| | | ATTTAAAACTTCATTTTTAATTTAAAAGGATCTAGGTGAAGATCCTTTTTG |
| | | ATAATCTCATGACCAAAATCCCTTAACGTGAGTTTTCGTTCCACTGAGCGT |
| | | CAGACCCC |
| 152 | <Human FOXP3 | ATGCCCAACCCCAGGCCTGGCAAGCCCTCGGCCCCTTCCTTGGCCCTTGG |
| | 1st coding exon | CCCATCTCCTGGTGCATCGCCCAGCTGGAGGGCTGCCCCTAAAGCAAGCG |
| | sequences | ACCTGCTGGGGGCCCGGGGCCCGGGTGGCACGTTCCAGGGCCGAGATCTT |
| | included in AAV | CGAGGCGGGGCCCATGCCTCCTCTTCTTCCTTGAACCCCATGCCACCATC |
| | #3080 (modified | GCAGCTGCAG |
| | to be non- | |
| | cleavable by | |
| | TALEN, | |
| | Cas9/T3 or | |
| | Cas9/T4 or | |
| | Cas9/T9> | |
| 153 | <#3098 | GTAGAAAAGATCAAAGGATCTTCTTGAGATCCTTTTTTTCTGCGCGTAATC |
| | pAAV_FOXP3. | TGCTGCTTGCAAACAAAAAAACCACCGCTACCAGCGGTGGTTTGTTTGCC |
| | 06_MND.FOXP | GGATCAAGAGCTACCAACTCTTTTTCCGAAGGTAACTGGCTTCAGCAGAG |
| | 3geneartCDS.R3 | CGCAGATACCAAATACTGTCCTTCTAGTGTAGCCGTAGTTAGGCCACCAC |
| | 97W.P2A.LNGF | TTCAAGAACTCTGTAGCACCGCCTACATACCTCGCTCTGCTAATCCTGTTA |
| | R.pA_06_for | CCAGTGGCTGCTGCCAGTGGCGATAAGTCGTGTCTTACCGGGTTGGACTC |
| | T9> | AAGACGATAGTTACCGGATAAGGCGCAGCGGTCGGGCTGAACGGGGGGT |
| | | TCGTGCACACAGCCCAGCTTGGAGCGAACGACCTACACCGAACTGAGAT |
| | | ACCTACAGCGTGAGCTATGAGAAAGCGCCACGCTTCCCGAAGGGAGAAA |
| | | GGCGGACAGGTATCCGGTAAGCGGCAGGGTCGGAACAGGAGAGCGCACG |
| | | AGGGAGCTTCCAGGGGGAAACGCCTGGTATCTTTATAGTCCTGTCGGGTT |
| | | TCGCCACCTCTGACTTGAGCGTCGATTTTTGTGATGCTCGTCAGGGGGGC |
| | | GGAGCCTATGGAAAAACGCCAGCAACGCGGCCTTTTTACGGTTCCTGGCC |
| | | TTTTGCTGGCCTTTTGCTCACATGTTCTTTCCTGCGTTATCCCCTGATTCTG |
| | | TGGATAACCGTATTACCGCCTTTGAGTGAGCTGATACCGCTCGCCGCAGC |
| | | CGAACGACCGAGCGCAGCGAGTCAGTGAGCGAGGAAGCGGAAGAGCGC |
| | | CCAATACGCAAACCGCCTCTCCCCGCGCGTTGGCCGATTCATTAATGCAG |
| | | CTGCGCGCTCGCTCGCTCACTGAGGCCGCCCGGGCAAAGCCCGGGCGTCG |
| | | GGCGACCTTTGGTCGCCCGGCCTCAGTGAGCGAGCGAGCGCGCAGAGAG |
| | | GGAGTGGCCAACTCCATCACTAGGGGTTCCTTGTAGTTAATGATTAACCC |
| | | GCCATGCTACTTATCTACGTAGCGGCCGCATCACTTGCCAGGACTGTTAC |
| | | AATAGCCTCCTCACTAGCCCCACTCACAGCAGCCAGATGAATCTTTTGAG |
| | | TCCATGCCTAGTCACTGGGGCAAAATAGGACTCCGAGGAGAAAGTCCGA |
| | | GACCAGCTCCGGCAAGATGAGCAAACACAGCCTGTGCAGGGTGCAGGGA |
| | | GGGCTAGAGGCCTGAGGCTTGAAACAGCTCTCAAGTGGAGGGGGAAACA |
| | | ACCATTGCCCTCATAGAGGACACATCCACACCAGGGCTGTGCTAGCGTGG |
| | | GCAGGCAAGCCAGGTGCTGGACCTCTGCACGTGGGGCATGTGTGGGTAT |
| | | GTACATGTACCTGTGTTCTTGGTGTGTGTGTGTGTGTGTGTGTGTGTGTGT |
| | | GTCTAGAGCTGGGGTGCAACTATGGGGCCCCTCGGGACATGTCCCAGCCA |
| | | ATGCCTGCTTTGACCAGAGGAGTGTCCACGTGGCTCAGGTGGTCGAGTAT |
| | | CTCATACCGCCCTAGCACACGTGTGACTCCTTTCCCCTATTGTCTACGCAG |
| | | CCTGCCCTTGGACAAGGACCCGATGCCCAACCCCAGGCCTGGCAAGCCCT |
| | | CGGCCCCTTCCTTGGCCCTTGGCCCATCCCCACGCGTAGGAACAGAGAAA |
| | | CAGGAGAATATGGGCCAAACAGGATATCTGTGGTAAGCAGTTCCTGCCCC |
| | | GGCTCAGGGCCAAGAACAGTTGGAACAGCAGAATATGGGCCAAACAGGA |
| | | TATCTGTGGTAAGCAGTTCCTGCCCCGGCTCAGGGCCAAGAACAGATGGT |
| | | CCCCAGATGCGGTCCCGCCCTCAGCAGTTTCTAGAGAACCATCAGATGTT |
| | | TCCAGGGTGCCCCAAGGACCTGAAATGACCCTGTGCCTTATTTGAACTAA |
| | | CCAATCAGTTCGCTTCTCGCTTCTGTTCGCGCGCTTCTGCTCCCCGAGCTC |
| | | TATATAAGCAGAGCTCGTTTAGTGAACCGTCAGATCGCCTGGAGACGCCA |
| | | TCCACGCTGTTTTGACTTCCATAGAAGGATCTCGAGGCCACCATGCCTAA |
| | | TCCTCGGCCTGGAAAGCCTAGCGCTCCTTCTCTTGCTCTGGGACCTTCTCC |
| | | TGGCGCCTCTCCATCTTGGAGAGCCGCTCCTAAAGCCAGCGATCTGCTGG |
| | | GAGCTAGAGGACCTGGCGGCACATTTCAGGGCAGAGATCTTAGAGGCGG |
| | | AGCCCACGCTAGCTCCTCCAGCCTTAATCCTATGCCTCCTAGCCAGCTCCA |
| | | GCTGCCTACACTGCCTCTGGTTATGGTGGCTCCTAGCGGAGCTAGACTGG |
| | | GCCCTCTGCCTCATCTGCAAGCTCTGCTGCAGGACAGACCCCACTTCATG |
| | | CACCAGCTGAGCACCGTGGATGCCCACGCAAGAACACCTGTGCTGCAGG |
| | | TTCACCCTCTGGAATCCCCAGCCATGATCAGCCTGACACCTCCAACAACA |
| | | GCCACCGGCGTGTTCAGCCTGAAAGCCAGACCTGGACTGCCTCCTGGCAT |
| | | CAATGTGGCCAGCCTGGAATGGGTGTCCAGAGAACCTGCTCTGCTGTGCA |
| | | CATTCCCCAATCCAAGCGCTCCCAGAAAGGACAGCACACTGTCTGCCGTG |
| | | CCTCAGAGCAGCTATCCCCTGCTTGCTAACGGCGTGTGCAAGTGGCCTGG |
| | | ATGCGAGAAGGTGTTCGAGGAACCCGAGGACTTCCTGAAGCACTGCCAG |
| | | GCCGATCATCTGCTGGACGAGAAAGGCAGAGCCCAGTGTCTGCTCCAGC |
| | | GCGAGATGGTGCAGTCTCTGGAACAGCAGCTGGTCCTGGAAAAAGAAAA |
| | | GCTGAGCGCCATGCAGGCCCACCTGGCCGGAAAAATGGCCCTGACAAAG |
| | | GCCAGCAGCGTGGCCTCTTCTGATAAGGGCAGCTGCTGCATTGTGGCCGC |
| | | TGGATCTCAGGGACCTGTGGTTCCTGCTTGGAGCGGACCTAGAGAGGCCC |
| | | CTGATTCTCTGTTTGCCGTGCGGAGACACCTGTGGGGCTCTCACGGCAAC |
| | | TCTACTTTCCCCGAGTTCCTGCACAACATGGACTACTTCAAGTTCCACAAC |
| | | ATGCGGCCTCCATTCACCTACGCCACACTGATCAGATGGGCCATTCTGGA |
| | | AGCCCCTGAGAAGCAGAGAACCCTGAACGAGATCTACCACTGGTTTACCC |
| | | GGATGTTCGCCTTCTTCCGGAATCACCCTGCCACCTGGAAGAACGCCATC |
| | | CGGCACAATCTGAGCCTGCACAAGTGCTTCGTGTGGGTGGAATCTGAGAA |
| | | AGGCGCCGTGTGGACAGTGGACGAGCTGGAATTCAGAAAGAAGAGAAGC |
| | | CAGCGGCCTAGCCGGTGCAGCAATCCTACACCTGGACCTGGAAGCGGAG |
| | | CGACTAACTTCAGCCTGCTGAAGCAGGCCGGAGATGTGGAGGAAAACCC |
| | | TGGACCGATGGGGGCAGGTGCCACCGGACGAGCCATGGACGGGCCGCGC |
| | | CTGCTGCTGTTGCTGCTTCTGGGGGTGTCCCTTGGAGGTGCCAAGGAGGC |
| | | ATGCCCCACAGGCCTGTACACACACAGCGGTGAGTGCTGCAAAGCCTGC |
| | | AACCTGGGCGAGGGTGTGGCCCAGCCTTGTGGAGCCAACCAGACCGTGT |
| | | GTGAGCCCTGCCTGGACAGCGTGACGTTCTCCGACGTGGTGAGCGCGACC |
| | | GAGCCGTGCAAGCCGTGCACCGAGTGCGTGGGGCTCCAGAGCATGTCGG |
| | | CGCCGTGCGTGGAGGCCGACGACGCCGTGTGCCGCTGCGCCTACGGCTAC |
| | | TACCAGGATGAGACGACTGGGCGCTGCGAGGCGTGCCGCGTGTGCGAGG |
| | | CGGGCTCGGGCCTCGTGTTCTCCTGCCAGGACAAGCAGAACACCGTGTGC |
| | | GAGGAGTGCCCCGACGGCACGTATTCCGACGAGGCCAACCACGTGGACC |
| | | CGTGCCTGCCCTGCACCGTGTGCGAGGACACCGAGCGCCAGCTCCGCGAG |
| | | TGCACACGCTGGGCCGACGCCGAGTGCGAGGAGATCCCTGGCCGTTGGA |
| | | TTACACGGTCCACACCCCCAGAGGGCTCGGACAGCACAGCCCCCAGCAC |
| | | CCAGGAGCCTGAGGCACCTCCAGAACAAGACCTCATAGCCAGCACGGTG |
| | | GCAGGTGTGGTGACCACAGTGATGGGCAGCTCCCAGCCCGTGGTGACCC |
| | | GAGGCACCACCGACAACCTCATCCCTGTCTATTGCTCCATCCTGGCTGCT |
| | | GTGGTTGTGGGTCTTGTGGCCTACATAGCCTTCAAGAGGTGAAAGCTTGT |
| | | CGACTGCTTTATTTGTGAAATTTGTGATGCTATTGCTTTATTTGTAACCAT |
| | | TATAAGCTGCAATAAACAAGTTAACAACAACAATTGCATTCATTTTATGT |
| | | TTCAGGTTCAGGGGGAGATGTGGGAGGTTTTTTAAAGCACTAGTGCCTCG |
| | | CCCAGCTGGAGGGCTGCACCCAAAGCCTCAGACCTGCTGGGGGCCCGGG |
| | | GCCCAGGGGGAACCTTCCAGGGCCGAGATCTTCGAGGCGGGGCCCATGC |
| | | CTCCTCTTCTTCCTTGAACCCCATGCCACCATCGCAGCTGCAGGTGAGGCC |
| | | CTGGGCCCAGGATGGGGCAGGCAGGGTGGGGTACCTGGACCTACAGGTG |
| | | CCGACCTTTACTGTGGCACTGGGCGGGAGGGGGGCTGGCTGGGGCACAG |
| | | GAAGTGGTTTCTGGGTCCCAGGCAAGTCTGTGACTTATGCAGATGTTGCA |
| | | GGGCCAAGAAAATCCCCACCTGCCAGGCCTCAGAGATTGGAGGCTCTCCC |
| | | CGACCTCCCAATCCCTGTCTCAGGAGAGGAGGAGGCCGTATTGTAGTCCC |
| | | ATGAGCATAGCTATGTGTCCCCATCCCCATGTGACAAGAGAAGAGGACTG |
| | | GGGCCAAGTAGGTGAGGTGACAGGGCTGAGGCCAGCTCTGCAACTTATT |
| | | AGCTGTTTGATCTTTAAAAAGTTACTCGATCTCCATGAGCCTCAGTTTCCA |
| | | TACGTGTAAAAGGGGGATGATCATAGCATCTACCATGTGGGCTTGCAGGA |
| | | TCCTACGTAGATAAGTAGCATGGCGGGTTAATCATTAACTACAAGGAACC |
| | | CCTAGTGATGGAGTTGGCCACTCCCTCTCTGCGCGCTCGCTCGCTCACTGA |
| | | GGCCGGGCGACCAAAGGTCGCCCGACGCCCGGGCTTTGCCCGGGCGGCC |
| | | TCAGTGAGCGAGCGAGCGCGCCAGCTGGCGTAATAGCGAAGAGGCCCGC |
| | | ACCGATCGCCCTTCCCAACAGTTGCGCAGCCTGAATGGCGAATGGCGATT |
| | | CCGTTGCAATGGCTGGCGGTAATATTGTTCTGGATATTACCAGCAAGGCC |
| | | GATAGTTTGAGTTCTTCTACTCAGGCAAGTGATGTTATTACTAATCAAAG |
| | | AAGTATTGCGACAACGGTTAATTTGCGTGATGGACAGACTCTTTTACTCG |
| | | GTGGCCTCACTGATTATAAAAACACTTCTCAGGATTCTGGCGTACCGTTC |
| | | CTGTCTAAAATCCCTTTAATCGGCCTCCTGTTTAGCTCCCGCTCTGATTCT |
| | | AACGAGGAAAGCACGTTATACGTGCTCGTCAAAGCAACCATAGTACGCG |
| | | CCCTGTAGCGGCGCATTAAGCGCGGCGGGTGTGGTGGTTACGCGCAGCGT |
| | | GACCGCTACACTTGCCAGCGCCCTAGCGCCCGCTCCTTTCGCTTTCTTCCC |
| | | TTCCTTTCTCGCCACGTTCGCCGGCTTTCCCCGTCAAGCTCTAAATCGGGG |
| | | GCTCCCTTTAGGGTTCCGATTTAGTGCTTTACGGCACCTCGACCCCAAAA |
| | | AACTTGATTAGGGTGATGGTTCACGTAGTGGGCCATCGCCCTGATAGACG |
| | | GTTTTTCGCCCTTTGACGTTGGAGTCCACGTTCTTTAATAGTGGACTCTTG |
| | | TTCCAAACTGGAACAACACTCAACCCTATCTCGGTCTATTCTTTTGATTTA |
| | | TAAGGGATTTTGCCGATTTCGGCCTATTGGTTAAAAAATGAGCTGATTTA |
| | | ACAAAAATTTAACGCGAATTTTAACAAAATATTAACGTTTACAATTTAAA |
| | | TATTTGCTTATACAATCTTCCTGTTTTTGGGGCTTTTCTGATTATCAACCGG |
| | | GGTACATATGATTGACATGCTAGTTTTACGATTACCGTTCATCGATTCTCT |
| | | TGTTTGCTCCAGACTCTCAGGCAATGACCTGATAGCCTTTGTAGAGACCT |
| | | CTCAAAAATAGCTACCCTCTCCGGCATGAATTTATCAGCTAGAACGGTTG |
| | | AATATCATATTGATGGTGATTTGACTGTCTCCGGCCTTTCTCACCCGTTTG |
| | | AATCTTTACCTACACATTACTCAGGCATTGCATTTAAAATATATGAGGGTT |
| | | CTAAAAATTTTTATCCTTGCGTTGAAATAAAGGCTTCTCCCGCAAAAGTA |
| | | TTACAGGGTCATAATGTTTTTGGTACAACCGATTTAGCTTTATGCTCTGAG |
| | | GCTTTATTGCTTAATTTTGCTAATTCTTTGCCTTGCCTGTATGATTTATTGG |
| | | ATGTTGGAATCGCCTGATGCGGTATTTTCTCCTTACGCATCTGTGCGGTAT |
| | | TTCACACCGCATATGGTGCACTCTCAGTACAATCTGCTCTGATGCCGCAT |
| | | AGTTAAGCCAGCCCCGACACCCGCCAACACCCGCTGACGCGCCCTGACG |
| | | GGCTTGTCTGCTCCCGGCATCCGCTTACAGACAAGCTGTGACCGTCTCCG |
| | | GGAGCTGCATGTGTCAGAGGTTTTCACCGTCATCACCGAAACGCGCGAGA |
| | | CGAAAGGGCCTCGTGATACGCCTATTTTTATAGGTTAATGTCATGATAAT |
| | | AATGGTTTCTTAGACGTCAGGTGGCACTTTTCGGGGAAATGTGCGCGGAA |
| | | CCCCTATTTGTTTATTTTTCTAAATACATTCAAATATGTATCCGCTCATGA |
| | | GACAATAACCCTGATAAATGCTTCAATAATATTGAAAAAGGAAGAGTAT |
| | | GAGTATTCAACATTTCCGTGTCGCCCTTATTCCCTTTTTTGCGGCATTTTGC |
| | | CTTCCTGTTTTTGCTCACCCAGAAACGCTGGTGAAAGTAAAAGATGCTGA |
| | | AGATCAGTTGGGTGCACGAGTGGGTTACATCGAACTGGATCTCAACAGCG |
| | | GTAAGATCCTTGAGAGTTTTCGCCCCGAAGAACGTTTTCCAATGATGAGC |
| | | ACTTTTAAAGTTCTGCTATGTGGCGCGGTATTATCCCGTATTGACGCCGGG |
| | | CAAGAGCAACTCGGTCGCCGCATACACTATTCTCAGAATGACTTGGTTGA |
| | | GTACTCACCAGTCACAGAAAAGCATCTTACGGATGGCATGACAGTAAGA |
| | | GAATTATGCAGTGCTGCCATAACCATGAGTGATAACACTGCGGCCAACTT |
| | | ACTTCTGACAACGATCGGAGGACCGAAGGAGCTAACCGCTTTTTTGCACA |
| | | ACATGGGGGATCATGTAACTCGCCTTGATCGTTGGGAACCGGAGCTGAAT |
| | | GAAGCCATACCAAACGACGAGCGTGACACCACGATGCCTGTAGCAATGG |
| | | CAACAACGTTGCGCAAACTATTAACTGGCGAACTACTTACTCTAGCTTCC |
| | | CGGCAACAATTAATAGACTGGATGGAGGCGGATAAAGTTGCAGGACCAC |
| | | TTCTGCGCTCGGCCCTTCCGGCTGGCTGGTTTATTGCTGATAAATCTGGAG |
| | | CCGGTGAGCGTGGGTCTCGCGGTATCATTGCAGCACTGGGGCCAGATGGT |
| | | AAGCCCTCCCGTATCGTAGTTATCTACACGACGGGGAGTCAGGCAACTAT |
| | | GGATGAACGAAATAGACAGATCGCTGAGATAGGTGCCTCACTGATTAAG |
| | | CATTGGTAACTGTCAGACCAAGTTTACTCATATATACTTTAGATTGATTTA |
| | | AAACTTCATTTTTAATTTAAAAGGATCTAGGTGAAGATCCTTTTTGATAAT |
| | | CTCATGACCAAAATCCCTTAACGTGAGTTTTCGTTCCACTGAGCGTCAGA |
| | | CCCC |
| 154 | <#3132_pAAV_ | GTAGAAAAGATCAAAGGATCTTCTTGAGATCCTTTTTTTCTGCGCGTAATC |
| | FOXP3.06_MN | TGCTGCTTGCAAACAAAAAAACCACCGCTACCAGCGGTGGTTTGTTTGCC |
| | D.FOXP3genear | GGATCAAGAGCTACCAACTCTTTTTCCGAAGGTAACTGGCTTCAGCAGAG |
| | tCDS.P2A.LNG | CGCAGATACCAAATACTGTTCTTCTAGTGTAGCCGTAGTTAGGCCACCAC |
| | FR.pA_06_for | TTCAAGAACTCTGTAGCACCGCCTACATACCTCGCTCTGCTAATCCTGTTA |
| | T9.kanamycin> | CCAGTGGCTGCTGCCAGTGGCGATAAGTCGTGTCTTACCGGGTTGGACTC |
| | a.k.a. 3066kanam | AAGACGATAGTTACCGGATAAGGCGCAGCGGTCGGGCTGAACGGGGGGT |
| | ycin | TCGTGCACACAGCCCAGCTTGGAGCGAACGACCTACACCGAACTGAGAT |
| | | ACCTACAGCGTGAGCTATGAGAAAGCGCCACGCTTCCCGAAGGGAGAAA |
| | | GGCGGACAGGTATCCGGTAAGCGGCAGGGTCGGAACAGGAGAGCGCACG |
| | | AGGGAGCTTCCAGGGGGAAACGCCTGGTATCTTTATAGTCCTGTCGGGTT |
| | | TCGCCACCTCTGACTTGAGCGTCGATTTTTGTGATGCTCGTCAGGGGGGC |
| | | GGAGCCTATGGAAAAACGCCAGCAACGCGGCCTTTTTACGGTTCCTGGCC |
| | | TTTTGCTGGCCTTTTGCTCACATGTTCTTTCCTGCGTTATCCCCTGATTCTG |
| | | TGGATAACCGTATTACCGCCTTTGAGTGAGCTGATACCGCTCGCCGCAGC |
| | | CGAACGACCGAGCGCAGCGAGTCAGTGAGCGAGGAAGCGGAAGAGCGC |
| | | CCAATACGCAAACCGCCTCTCCCCGCGCGTTGGCCGATTCATTAATGCAG |
| | | CTGCGCGCTCGCTCGCTCACTGAGGCCGCCCGGGCAAAGCCCGGGCGTCG |
| | | GGCGACCTTTGGTCGCCCGGCCTCAGTGAGCGAGCGAGCGCGCAGAGAG |
| | | GGAGTGGCCAACTCCATCACTAGGGGTTCCTTGTAGTTAATGATTAACCC |
| | | GCCATGCTACTTATCTACGTAGCGGCCGCATCACTTGCCAGGACTGTTAC |
| | | AATAGCCTCCTCACTAGCCCCACTCACAGCAGCCAGATGAATCTTTTGAG |
| | | TCCATGCCTAGTCACTGGGGCAAAATAGGACTCCGAGGAGAAAGTCCGA |
| | | GACCAGCTCCGGCAAGATGAGCAAACACAGCCTGTGCAGGGTGCAGGGA |
| | | GGGCTAGAGGCCTGAGGCTTGAAACAGCTCTCAAGTGGAGGGGGAAACA |
| | | ACCATTGCCCTCATAGAGGACACATCCACACCAGGGCTGTGCTAGCGTGG |
| | | GCAGGCAAGCCAGGTGCTGGACCTCTGCACGTGGGGCATGTGTGGGTAT |
| | | GTACATGTACCTGTGTTCTTGGTGTGTGTGTGTGTGTGTGTGTGTGTGTGT |
| | | GTCTAGAGCTGGGGTGCAACTATGGGGCCCCTCGGGACATGTCCCAGCCA |
| | | ATGCCTGCTTTGACCAGAGGAGTGTCCACGTGGCTCAGGTGGTCGAGTAT |
| | | CTCATACCGCCCTAGCACACGTGTGACTCCTTTCCCCTATTGTCTACGCAG |
| | | CCTGCCCTTGGACAAGGACCCGATGCCCAACCCCAGGCCTGGCAAGCCCT |
| | | CGGCCCCTTCCTTGGCCCTTGGCCCATCCCCACGCGTAGGAACAGAGAAA |
| | | CAGGAGAATATGGGCCAAACAGGATATCTGTGGTAAGCAGTTCCTGCCCC |
| | | GGCTCAGGGCCAAGAACAGTTGGAACAGCAGAATATGGGCCAAACAGGA |
| | | TATCTGTGGTAAGCAGTTCCTGCCCCGGCTCAGGGCCAAGAACAGATGGT |
| | | CCCCAGATGCGGTCCCGCCCTCAGCAGTTTCTAGAGAACCATCAGATGTT |
| | | TCCAGGGTGCCCCAAGGACCTGAAATGACCCTGTGCCTTATTTGAACTAA |
| | | CCAATCAGTTCGCTTCTCGCTTCTGTTCGCGCGCTTCTGCTCCCCGAGCTC |
| | | TATATAAGCAGAGCTCGTTTAGTGAACCGTCAGATCGCCTGGAGACGCCA |
| | | TCCACGCTGTTTTGACTTCCATAGAAGGATCTCGAGGCCACCATGCCTAA |
| | | TCCTCGGCCTGGAAAGCCTAGCGCTCCTTCTCTTGCTCTGGGACCTTCTCC |
| | | TGGCGCCTCTCCATCTTGGAGAGCCGCTCCTAAAGCCAGCGATCTGCTGG |
| | | GAGCTAGAGGACCTGGCGGCACATTTCAGGGCAGAGATCTTAGAGGCGG |
| | | AGCCCACGCTAGCTCCTCCAGCCTTAATCCTATGCCTCCTAGCCAGCTCCA |
| | | GCTGCCTACACTGCCTCTGGTTATGGTGGCTCCTAGCGGAGCTAGACTGG |
| | | GCCCTCTGCCTCATCTGCAAGCTCTGCTGCAGGACAGACCCCACTTCATG |
| | | CACCAGCTGAGCACCGTGGATGCCCACGCAAGAACACCTGTGCTGCAGG |
| | | TTCACCCTCTGGAATCCCCAGCCATGATCAGCCTGACACCTCCAACAACA |
| | | GCCACCGGCGTGTTCAGCCTGAAAGCCAGACCTGGACTGCCTCCTGGCAT |
| | | CAATGTGGCCAGCCTGGAATGGGTGTCCAGAGAACCTGCTCTGCTGTGCA |
| | | CATTCCCCAATCCAAGCGCTCCCAGAAAGGACAGCACACTGTCTGCCGTG |
| | | CCTCAGAGCAGCTATCCCCTGCTTGCTAACGGCGTGTGCAAGTGGCCTGG |
| | | ATGCGAGAAGGTGTTCGAGGAACCCGAGGACTTCCTGAAGCACTGCCAG |
| | | GCCGATCATCTGCTGGACGAGAAAGGCAGAGCCCAGTGTCTGCTCCAGC |
| | | GCGAGATGGTGCAGTCTCTGGAACAGCAGCTGGTCCTGGAAAAAGAAAA |
| | | GCTGAGCGCCATGCAGGCCCACCTGGCCGGAAAAATGGCCCTGACAAAG |
| | | GCCAGCAGCGTGGCCTCTTCTGATAAGGGCAGCTGCTGCATTGTGGCCGC |
| | | TGGATCTCAGGGACCTGTGGTTCCTGCTTGGAGCGGACCTAGAGAGGCCC |
| | | CTGATTCTCTGTTTGCCGTGCGGAGACACCTGTGGGGCTCTCACGGCAAC |
| | | TCTACTTTCCCCGAGTTCCTGCACAACATGGACTACTTCAAGTTCCACAAC |
| | | ATGCGGCCTCCATTCACCTACGCCACACTGATCAGATGGGCCATTCTGGA |
| | | AGCCCCTGAGAAGCAGAGAACCCTGAACGAGATCTACCACTGGTTTACCC |
| | | GGATGTTCGCCTTCTTCCGGAATCACCCTGCCACCTGGAAGAACGCCATC |
| | | CGGCACAATCTGAGCCTGCACAAGTGCTTCGTGCGCGTGGAATCTGAGAA |
| | | AGGCGCCGTGTGGACAGTGGACGAGCTGGAATTCAGAAAGAAGAGAAGC |
| | | CAGCGGCCTAGCCGGTGCAGCAATCCTACACCTGGACCTGGAAGCGGAG |
| | | CGACTAACTTCAGCCTGCTGAAGCAGGCCGGAGATGTGGAGGAAAACCC |
| | | TGGACCGATGGGGGCAGGTGCCACCGGACGAGCCATGGACGGGCCGCGC |
| | | CTGCTGCTGTTGCTGCTTCTGGGGGTGTCCCTTGGAGGTGCCAAGGAGGC |
| | | ATGCCCCACAGGCCTGTACACACACAGCGGTGAGTGCTGCAAAGCCTGC |
| | | AACCTGGGCGAGGGTGTGGCCCAGCCTTGTGGAGCCAACCAGACCGTGT |
| | | GTGAGCCCTGCCTGGACAGCGTGACGTTCTCCGACGTGGTGAGCGCGACC |
| | | GAGCCGTGCAAGCCGTGCACCGAGTGCGTGGGGCTCCAGAGCATGTCGG |
| | | CGCCGTGCGTGGAGGCCGACGACGCCGTGTGCCGCTGCGCCTACGGCTAC |
| | | TACCAGGATGAGACGACTGGGCGCTGCGAGGCGTGCCGCGTGTGCGAGG |
| | | CGGGCTCGGGCCTCGTGTTCTCCTGCCAGGACAAGCAGAACACCGTGTGC |
| | | GAGGAGTGCCCCGACGGCACGTATTCCGACGAGGCCAACCACGTGGACC |
| | | CGTGCCTGCCCTGCACCGTGTGCGAGGACACCGAGCGCCAGCTCCGCGAG |
| | | TGCACACGCTGGGCCGACGCCGAGTGCGAGGAGATCCCTGGCCGTTGGA |
| | | TTACACGGTCCACACCCCCAGAGGGCTCGGACAGCACAGCCCCCAGCAC |
| | | CCAGGAGCCTGAGGCACCTCCAGAACAAGACCTCATAGCCAGCACGGTG |
| | | GCAGGTGTGGTGACCACAGTGATGGGCAGCTCCCAGCCCGTGGTGACCC |
| | | GAGGCACCACCGACAACCTCATCCCTGTCTATTGCTCCATCCTGGCTGCT |
| | | GTGGTTGTGGGTCTTGTGGCCTACATAGCCTTCAAGAGGTGAAAGCTTGT |
| | | CGACTGCTTTATTTGTGAAATTTGTGATGCTATTGCTTTATTTGTAACCAT |
| | | TATAAGCTGCAATAAACAAGTTAACAACAACAATTGCATTCATTTTATGT |
| | | TTCAGGTTCAGGGGGAGATGTGGGAGGTTTTTTAAAGCACTAGTGCCTCG |
| | | CCCAGCTGGAGGGCTGCACCCAAAGCCTCAGACCTGCTGGGGGCCCGGG |
| | | GCCCAGGGGGAACCTTCCAGGGCCGAGATCTTCGAGGCGGGGCCCATGC |
| | | CTCCTCTTCTTCCTTGAACCCCATGCCACCATCGCAGCTGCAGGTGAGGCC |
| | | CTGGGCCCAGGATGGGGCAGGCAGGGTGGGGTACCTGGACCTACAGGTG |
| | | CCGACCTTTACTGTGGCACTGGGCGGGAGGGGGGCTGGCTGGGGCACAG |
| | | GAAGTGGTTTCTGGGTCCCAGGCAAGTCTGTGACTTATGCAGATGTTGCA |
| | | GGGCCAAGAAAATCCCCACCTGCCAGGCCTCAGAGATTGGAGGCTCTCCC |
| | | CGACCTCCCAATCCCTGTCTCAGGAGAGGAGGAGGCCGTATTGTAGTCCC |
| | | ATGAGCATAGCTATGTGTCCCCATCCCCATGTGACAAGAGAAGAGGACTG |
| | | GGGCCAAGTAGGTGAGGTGACAGGGCTGAGGCCAGCTCTGCAACTTATT |
| | | AGCTGTTTGATCTTTAAAAAGTTACTCGATCTCCATGAGCCTCAGTTTCCA |
| | | TACGTGTAAAAGGGGGATGATCATAGCATCTACCATGTGGGCTTGCAGGA |
| | | TCCTACGTAGATAAGTAGCATGGCGGGTTAATCATTAACTACAAGGAACC |
| | | CCTAGTGATGGAGTTGGCCACTCCCTCTCTGCGCGCTCGCTCGCTCACTGA |
| | | GGCCGGGCGACCAAAGGTCGCCCGACGCCCGGGCTTTGCCCGGGCGGCC |
| | | TCAGTGAGCGAGCGAGCGCGCAGCTGGCGTAATAGCGAAGAGGCCCGCA |
| | | CCGATCGCCCTTCCCAACAGTTGCGCAGCCTGAATGGCGAATGGCGATTC |
| | | CGTTGCAATGGCTGGCGGTAATATTGTTCTGGATATTACCAGCAAGGCCG |
| | | ATAGTTTGAGTTCTTCTACTCAGGCAAGTGATGTTATTACTAATCAAAGA |
| | | AGTATTGCGACAACGGTTAATTTGCGTGATGGACAGACTCTTTTACTCGG |
| | | TGGCCTCACTGATTATAAAAACACTTCTCAGGATTCTGGCGTACCGTTCCT |
| | | GTCTAAAATCCCTTTAATCGGCCTCCTGTTTAGCTCCCGCTCTGATTCTAA |
| | | CGAGGAAAGCACGTTATACGTGCTCGTCAAAGCAACCATAGTACGCGCC |
| | | CTGTAGCGGCGCATTAAGCGCGGCGGGTGTGGTGGTTACGCGCAGCGTG |
| | | ACCGCTACACTTGCCAGCGCCCTAGCGCCCGCTCCTTTCGCTTTCTTCCCT |
| | | TCCTTTCTCGCCACGTTCGCCGGCTTTCCCCGTCAAGCTCTAAATCGGGGG |
| | | CTCCCTTTAGGGTTCCGATTTAGTGCTTTACGGCACCTCGACCCCAAAAA |
| | | ACTTGATTAGGGTGATGGTTCACGTAGTGGGCCATCGCCCTGATAGACGG |
| | | TTTTTCGCCCTTTGACGTTGGAGTCCACGTTCTTTAATAGTGGACTCTTGT |
| | | TCCAAACTGGAACAACACTCAACCCTATCTCGGTCTATTCTTTTGATTTAT |
| | | AAGGGATTTTGCCGATTTCGGCCTATTGGTTAAAAAATGAGCTGATTTAA |
| | | CAAAAATTTAACGCGAATTTTAACAAAATATTAACGTCTACAATTTAAAT |
| | | ATTTGCTTATACAATCTTCCTGTTTTTGGGGCTTTTCTGATTATCAACCGG |
| | | GGTACATATGATTGACATGCTAGTTTTACGATTACCGTTCATCGATTCTCT |
| | | TGTTTGCTCCAGACTCTCAGGCAATGACCTGATAGCCTTTGTAGAGACCT |
| | | CTCAAAAATAGCTACCCTCTCCGGCATGAATTTATCAGCTAGAACGGTTG |
| | | AATATCATATTGATGGTGATTTGACTGTCTCCGGCCTTTCTCACCCGTTTG |
| | | AATCTTTACCTACACATTACTCAGGCATTGCATTTAAAATATATGAGGGTT |
| | | CTAAAAATTTTTATCCTTGCGTTGAAATAAAGGCTTCTCCCGCAAAAGTA |
| | | TTACAGGGTCATAATGTTTTTGGTACAACCGATTTAGCTTTATGCTCTGAG |
| | | GCTTTATTGCTTAATTTTGCTAATTCTTTGCCTTGCCTGTATGATTTATTGG |
| | | ATGTTGGAATCGCCTGATGCGGTATTTTCTCCTTACGCATCTGTGCGGTAT |
| | | TTCACACCGCATATGGTGCACTCTCAGTACAATCTGCTCTGATGCCGCAT |
| | | AGTTAAGCCAGCCCCGACACCCGCCAACACCCGCTGACGCGCCCTGACG |
| | | GGCTTGTCTGCTCCCGGCATCCGCTTACAGACAAGCTGTGACCGTCTCCG |
| | | GGAGCTGCATGTGTCAGAGGTTTTCACCGTCATCACCGAAACGCGCGAGA |
| | | CGAAAGGGCCTCGTGATACGCCTATTTTTATAGGTTAATGTCATGAACAA |
| | | TAAAACTGTCTGCTTACATAAACAGTAATACAAGGGGTGTTATGAGCCAT |
| | | ATTCAACGGGAAACGTCGAGGCCGCGATTAAATTCCAACATGGATGCTG |
| | | ATTTATATGGGTATAAATGGGCTCGCGATAATGTCGGGCAATCAGGTGCG |
| | | ACAATCTATCGCTTGTATGGGAAGCCCGATGCGCCAGAGTTGTTTCTGAA |
| | | ACATGGCAAAGGTAGCGTTGCCAATGATGTTACAGATGAGATGGTCAGA |
| | | CTAAACTGGCTGACGGAATTTATGCCTCTTCCGACCATCAAGCATTTTATC |
| | | CGTACTCCTGATGATGCATGGTTACTCACCACTGCGATCCCCGGAAAAAC |
| | | AGCATTCCAGGTATTAGAAGAATATCCTGATTCAGGTGAAAATATTGTTG |
| | | ATGCGCTGGCAGTGTTCCTGCGCCGGTTGCATTCGATTCCTGTTTGTAATT |
| | | GTCCTTTTAACAGCGATCGCGTATTTCGTCTCGCTCAGGCGCAATCACGA |
| | | ATGAATAACGGTTTGGTTGATGCGAGTGATTTTGATGACGAGCGTAATGG |
| | | CTGGCCTGTTGAACAAGTCTGGAAAGAAATGCATAAACTTTTGCCATTCT |
| | | CACCGGATTCAGTCGTCACTCATGGTGATTTCTCACTTGATAACCTTATTT |
| | | TTGACGAGGGGAAATTAATAGGTTGTATTGATGTTGGACGAGTCGGAATC |
| | | GCAGACCGATACCAGGATCTTGCCATCCTATGGAACTGCCTCGGTGAGTT |
| | | TTCTCCTTCATTACAGAAACGGCTTTTTCAAAAATATGGTATTGATAATCC |
| | | TGATATGAATAAATTGCAGTTTCATTTGATGCTCGATGAGTTTTTCTAATC |
| | | TCATGACCAAAATCCCTTAACGTGAGTTTTCGTTCCACTGAGCGTCAGAC |
| | | CCC |
| 155 | <#3117 | GTAGAAAAGATCAAAGGATCTTCTTGAGATCCTTTTTTTCTGCGCGTAATC |
| | pAAV_FOXP3. | TGCTGCTTGCAAACAAAAAAACCACCGCTACCAGCGGTGGTTTGTTTGCC |
| | 045_MND.LNG | GGATCAAGAGCTACCAACTCTTTTTCCGAAGGTAACTGGCTTCAGCAGAG |
| | FR-P2A- | CGCAGATACCAAATACTGTCCTTCTAGTGTAGCCGTAGTTAGGCCACCAC |
| | FOXP3geneartC | TTCAAGAACTCTGTAGCACCGCCTACATACCTCGCTCTGCTAATCCTGTTA |
| | DS.pA_045_for | CCAGTGGCTGCTGCCAGTGGCGATAAGTCGTGTCTTACCGGGTTGGACTC |
| | T9> | AAGACGATAGTTACCGGATAAGGCGCAGCGGTCGGGCTGAACGGGGGGT |
| | | TCGTGCACACAGCCCAGCTTGGAGCGAACGACCTACACCGAACTGAGAT |
| | | ACCTACAGCGTGAGCTATGAGAAAGCGCCACGCTTCCCGAAGGGAGAAA |
| | | GGCGGACAGGTATCCGGTAAGCGGCAGGGTCGGAACAGGAGAGCGCACG |
| | | AGGGAGCTTCCAGGGGGAAACGCCTGGTATCTTTATAGTCCTGTCGGGTT |
| | | TCGCCACCTCTGACTTGAGCGTCGATTTTTGTGATGCTCGTCAGGGGGGC |
| | | GGAGCCTATGGAAAAACGCCAGCAACGCGGCCTTTTTACGGTTCCTGGCC |
| | | TTTTGCTGGCCTTTTGCTCACATGTTCTTTCCTGCGTTATCCCCTGATTCTG |
| | | TGGATAACCGTATTACCGCCTTTGAGTGAGCTGATACCGCTCGCCGCAGC |
| | | CGAACGACCGAGCGCAGCGAGTCAGTGAGCGAGGAAGCGGAAGAGCGC |
| | | CCAATACGCAAACCGCCTCTCCCCGCGCGTTGGCCGATTCATTAATGCAG |
| | | CTGCGCGCTCGCTCGCTCACTGAGGCCGCCCGGGCAAAGCCCGGGCGTCG |
| | | GGCGACCTTTGGTCGCCCGGCCTCAGTGAGCGAGCGAGCGCGCAGAGAG |
| | | GGAGTGGCCAACTCCATCACTAGGGGTTCCTTGTAGTTAATGATTAACCC |
| | | GCCATGCTACTTATCTACGTAGCGGCCGCAGCCTGTGCAGGGTGCAGGGA |
| | | GGGCTAGAGGCCTGAGGCTTGAAACAGCTCTCAAGTGGAGGGGGAAACA |
| | | ACCATTGCCCTCATAGAGGACACATCCACACCAGGGCTGTGCTAGCGTGG |
| | | GCAGGCAAGCCAGGTGCTGGACCTCTGCACGTGGGGCATGTGTGGGTAT |
| | | GTACATGTACCTGTGTTCTTGGTGTGTGTGTGTGTGTGTGTGTGTGTGTGT |
| | | GTCTAGAGCTGGGGTGCAACTATGGGGCCCCTCGGGACATGTCCCAGCCA |
| | | ATGCCTGCTTTGACCAGAGGAGTGTCCACGTGGCTCAGGTGGTCGAGTAT |
| | | CTCATACCGCCCTAGCACACGTGTGACTCCTTTCCCCTATTGTCTACGCAG |
| | | CCTGCCCTTGGACAAGGACCCGATGCCCAACCCCAGGCCTGGCAAGCCCT |
| | | CGGCCCCTTCCTTGGCCCTTGGCCCATCCCCACGCGTAGGAACAGAGAAA |
| | | CAGGAGAATATGGGCCAAACAGGATATCTGTGGTAAGCAGTTCCTGCCCC |
| | | GGCTCAGGGCCAAGAACAGTTGGAACAGCAGAATATGGGCCAAACAGGA |
| | | TATCTGTGGTAAGCAGTTCCTGCCCCGGCTCAGGGCCAAGAACAGATGGT |
| | | CCCCAGATGCGGTCCCGCCCTCAGCAGTTTCTAGAGAACCATCAGATGTT |
| | | TCCAGGGTGCCCCAAGGACCTGAAATGACCCTGTGCCTTATTTGAACTAA |
| | | CCAATCAGTTCGCTTCTCGCTTCTGTTCGCGCGCTTCTGCTCCCCGAGCTC |
| | | TATATAAGCAGAGCTCGTTTAGTGAACCGTCAGATCGCCTGGAGACGCCA |
| | | TCCACGCTGTTTTGACTTCCATAGAAGGATCTCGAGGCCACCATGGGGGC |
| | | AGGTGCCACCGGACGAGCCATGGACGGGCCGCGCCTGCTGCTGTTGCTGC |
| | | TTCTGGGGGTGTCCCTTGGAGGTGCCAAGGAGGCATGCCCCACAGGCCTG |
| | | TACACACACAGCGGTGAGTGCTGCAAAGCCTGCAACCTGGGCGAGGGTG |
| | | TGGCCCAGCCTTGTGGAGCCAACCAGACCGTGTGTGAGCCCTGCCTGGAC |
| | | AGCGTGACGTTCTCCGACGTGGTGAGCGCGACCGAGCCGTGCAAGCCGT |
| | | GCACCGAGTGCGTGGGGCTCCAGAGCATGTCGGCGCCGTGCGTGGAGGC |
| | | CGACGACGCCGTGTGCCGCTGCGCCTACGGCTACTACCAGGATGAGACG |
| | | ACTGGGCGCTGCGAGGCGTGCCGCGTGTGCGAGGCGGGCTCGGGCCTCG |
| | | TGTTCTCCTGCCAGGACAAGCAGAACACCGTGTGCGAGGAGTGCCCCGAC |
| | | GGCACGTATTCCGACGAGGCCAACCACGTGGACCCGTGCCTGCCCTGCAC |
| | | CGTGTGCGAGGACACCGAGCGCCAGCTCCGCGAGTGCACACGCTGGGCC |
| | | GACGCCGAGTGCGAGGAGATCCCTGGCCGTTGGATTACACGGTCCACACC |
| | | CCCAGAGGGCTCGGACAGCACAGCCCCCAGCACCCAGGAGCCTGAGGCA |
| | | CCTCCAGAACAAGACCTCATAGCCAGCACGGTGGCAGGTGTGGTGACCA |
| | | CAGTGATGGGCAGCTCCCAGCCCGTGGTGACCCGAGGCACCACCGACAA |
| | | CCTCATCCCTGTCTATTGCTCCATCCTGGCTGCTGTGGTTGTGGGTCTTGT |
| | | GGCCTACATAGCCTTCAAGAGGGGAAGCGGAGCGACTAACTTCAGCCTG |
| | | CTGAAGCAGGCCGGAGATGTGGAGGAAAACCCTGGACCGATGCCTAATC |
| | | CTCGGCCTGGAAAGCCTAGCGCTCCTTCTCTTGCTCTGGGACCTTCTCCTG |
| | | GCGCCTCTCCATCTTGGAGAGCCGCTCCTAAAGCCAGCGATCTGCTGGGA |
| | | GCTAGAGGACCTGGCGGCACATTTCAGGGCAGAGATCTTAGAGGCGGAG |
| | | CCCACGCTAGCTCCTCCAGCCTTAATCCTATGCCTCCTAGCCAGCTCCAGC |
| | | TGCCTACACTGCCTCTGGTTATGGTGGCTCCTAGCGGAGCTAGACTGGGC |
| | | CCTCTGCCTCATCTGCAAGCTCTGCTGCAGGACAGACCCCACTTCATGCA |
| | | CCAGCTGAGCACCGTGGATGCCCACGCAAGAACACCTGTGCTGCAGGTTC |
| | | ACCCTCTGGAATCCCCAGCCATGATCAGCCTGACACCTCCAACAACAGCC |
| | | ACCGGCGTGTTCAGCCTGAAAGCCAGACCTGGACTGCCTCCTGGCATCAA |
| | | TGTGGCCAGCCTGGAATGGGTGTCCAGAGAACCTGCTCTGCTGTGCACAT |
| | | TCCCCAATCCAAGCGCTCCCAGAAAGGACAGCACACTGTCTGCCGTGCCT |
| | | CAGAGCAGCTATCCCCTGCTTGCTAACGGCGTGTGCAAGTGGCCTGGATG |
| | | CGAGAAGGTGTTCGAGGAACCCGAGGACTTCCTGAAGCACTGCCAGGCC |
| | | GATCATCTGCTGGACGAGAAAGGCAGAGCCCAGTGTCTGCTCCAGCGCG |
| | | AGATGGTGCAGTCTCTGGAACAGCAGCTGGTCCTGGAAAAAGAAAAGCT |
| | | GAGCGCCATGCAGGCCCACCTGGCCGGAAAAATGGCCCTGACAAAGGCC |
| | | AGCAGCGTGGCCTCTTCTGATAAGGGCAGCTGCTGCATTGTGGCCGCTGG |
| | | ATCTCAGGGACCTGTGGTTCCTGCTTGGAGCGGACCTAGAGAGGCCCCTG |
| | | ATTCTCTGTTTGCCGTGCGGAGACACCTGTGGGGCTCTCACGGCAACTCT |
| | | ACTTTCCCCGAGTTCCTGCACAACATGGACTACTTCAAGTTCCACAACAT |
| | | GCGGCCTCCATTCACCTACGCCACACTGATCAGATGGGCCATTCTGGAAG |
| | | CCCCTGAGAAGCAGAGAACCCTGAACGAGATCTACCACTGGTTTACCCGG |
| | | ATGTTCGCCTTCTTCCGGAATCACCCTGCCACCTGGAAGAACGCCATCCG |
| | | GCACAATCTGAGCCTGCACAAGTGCTTCGTGCGCGTGGAATCTGAGAAAG |
| | | GCGCCGTGTGGACAGTGGACGAGCTGGAATTCAGAAAGAAGAGAAGCCA |
| | | GCGGCCTAGCCGGTGCAGCAATCCTACACCTGGACCTTGAAAGCTTGTCG |
| | | ACTGCTTTATTTGTGAAATTTGTGATGCTATTGCTTTATTTGTAACCATTAT |
| | | AAGCTGCAATAAACAAGTTAACAACAACAATTGCATTCATTTTATGTTTC |
| | | AGGTTCAGGGGGAGATGTGGGAGGTTTTTTAAAGCACTAGTGCCTCGCCC |
| | | AGCTGGAGGGCTGCACCCAAAGCCTCAGACCTGCTGGGGGCCCGGGGCC |
| | | CAGGGGGAACCTTCCAGGGCCGAGATCTTCGAGGCGGGGCCCATGCCTC |
| | | CTCTTCTTCCTTGAACCCCATGCCACCATCGCAGCTGCAGGTGAGGCCCT |
| | | GGGCCCAGGATGGGGCAGGCAGGGTGGGGTACCTGGACCTACAGGTGCC |
| | | GACCTTTACTGTGGCACTGGGCGGGAGGGGGGCTGGCTGGGGCACAGGA |
| | | AGTGGTTTCTGGGTCCCAGGCAAGTCTGTGACTTATGCAGATGTTGCAGG |
| | | GCCAAGAAAATCCCCACCTGCCAGGCCTCAGAGATTGGAGGCTCTCCCCG |
| | | ACCTCCCAATCCCTGTCTCAGGAGAGGAGGAGGCCGTATTGTAGTCCCAT |
| | | GAGCATAGCTATGTGTCCCCATCCCCATGTGACAAGAGAAGAGGAGGAT |
| | | CCTACGTAGATAAGTAGCATGGCGGGTTAATCATTAACTACAAGGAACCC |
| | | CTAGTGATGGAGTTGGCCACTCCCTCTCTGCGCGCTCGCTCGCTCACTGA |
| | | GGCCGGGCGACCAAAGGTCGCCCGACGCCCGGGCTTTGCCCGGGCGGCC |
| | | TCAGTGAGCGAGCGAGCGCGCCAGCTGGCGTAATAGCGAAGAGGCCCGC |
| | | ACCGATCGCCCTTCCCAACAGTTGCGCAGCCTGAATGGCGAATGGCGATT |
| | | CCGTTGCAATGGCTGGCGGTAATATTGTTCTGGATATTACCAGCAAGGCC |
| | | GATAGTTTGAGTTCTTCTACTCAGGCAAGTGATGTTATTACTAATCAAAG |
| | | AAGTATTGCGACAACGGTTAATTTGCGTGATGGACAGACTCTTTTACTCG |
| | | GTGGCCTCACTGATTATAAAAACACTTCTCAGGATTCTGGCGTACCGTTC |
| | | CTGTCTAAAATCCCTTTAATCGGCCTCCTGTTTAGCTCCCGCTCTGATTCT |
| | | AACGAGGAAAGCACGTTATACGTGCTCGTCAAAGCAACCATAGTACGCG |
| | | CCCTGTAGCGGCGCATTAAGCGCGGCGGGTGTGGTGGTTACGCGCAGCGT |
| | | GACCGCTACACTTGCCAGCGCCCTAGCGCCCGCTCCTTTCGCTTTCTTCCC |
| | | TTCCTTTCTCGCCACGTTCGCCGGCTTTCCCCGTCAAGCTCTAAATCGGGG |
| | | GCTCCCTTTAGGGTTCCGATTTAGTGCTTTACGGCACCTCGACCCCAAAA |
| | | AACTTGATTAGGGTGATGGTTCACGTAGTGGGCCATCGCCCTGATAGACG |
| | | GTTTTTCGCCCTTTGACGTTGGAGTCCACGTTCTTTAATAGTGGACTCTTG |
| | | TTCCAAACTGGAACAACACTCAACCCTATCTCGGTCTATTCTTTTGATTTA |
| | | TAAGGGATTTTGCCGATTTCGGCCTATTGGTTAAAAAATGAGCTGATTTA |
| | | ACAAAAATTTAACGCGAATTTTAACAAAATATTAACGTTTACAATTTAAA |
| | | TATTTGCTTATACAATCTTCCTGTTTTTGGGGCTTTTCTGATTATCAACCGG |
| | | GGTACATATGATTGACATGCTAGTTTTACGATTACCGTTCATCGATTCTCT |
| | | TGTTTGCTCCAGACTCTCAGGCAATGACCTGATAGCCTTTGTAGAGACCT |
| | | CTCAAAAATAGCTACCCTCTCCGGCATGAATTTATCAGCTAGAACGGTTG |
| | | AATATCATATTGATGGTGATTTGACTGTCTCCGGCCTTTCTCACCCGTTTG |
| | | AATCTTTACCTACACATTACTCAGGCATTGCATTTAAAATATATGAGGGTT |
| | | CTAAAAATTTTTATCCTTGCGTTGAAATAAAGGCTTCTCCCGCAAAAGTA |
| | | TTACAGGGTCATAATGTTTTTGGTACAACCGATTTAGCTTTATGCTCTGAG |
| | | GCTTTATTGCTTAATTTTGCTAATTCTTTGCCTTGCCTGTATGATTTATTGG |
| | | ATGTTGGAATCGCCTGATGCGGTATTTTCTCCTTACGCATCTGTGCGGTAT |
| | | TTCACACCGCATATGGTGCACTCTCAGTACAATCTGCTCTGATGCCGCAT |
| | | AGTTAAGCCAGCCCCGACACCCGCCAACACCCGCTGACGCGCCCTGACG |
| | | GGCTTGTCTGCTCCCGGCATCCGCTTACAGACAAGCTGTGACCGTCTCCG |
| | | GGAGCTGCATGTGTCAGAGGTTTTCACCGTCATCACCGAAACGCGCGAGA |
| | | CGAAAGGGCCTCGTGATACGCCTATTTTTATAGGTTAATGTCATGATAAT |
| | | AATGGTTTCTTAGACGTCAGGTGGCACTTTTCGGGGAAATGTGCGCGGAA |
| | | CCCCTATTTGTTTATTTTTCTAAATACATTCAAATATGTATCCGCTCATGA |
| | | GACAATAACCCTGATAAATGCTTCAATAATATTGAAAAAGGAAGAGTAT |
| | | GAGTATTCAACATTTCCGTGTCGCCCTTATTCCCTTTTTTGCGGCATTTTGC |
| | | CTTCCTGTTTTTGCTCACCCAGAAACGCTGGTGAAAGTAAAAGATGCTGA |
| | | AGATCAGTTGGGTGCACGAGTGGGTTACATCGAACTGGATCTCAACAGCG |
| | | GTAAGATCCTTGAGAGTTTTCGCCCCGAAGAACGTTTTCCAATGATGAGC |
| | | ACTTTTAAAGTTCTGCTATGTGGCGCGGTATTATCCCGTATTGACGCCGGG |
| | | CAAGAGCAACTCGGTCGCCGCATACACTATTCTCAGAATGACTTGGTTGA |
| | | GTACTCACCAGTCACAGAAAAGCATCTTACGGATGGCATGACAGTAAGA |
| | | GAATTATGCAGTGCTGCCATAACCATGAGTGATAACACTGCGGCCAACTT |
| | | ACTTCTGACAACGATCGGAGGACCGAAGGAGCTAACCGCTTTTTTGCACA |
| | | ACATGGGGGATCATGTAACTCGCCTTGATCGTTGGGAACCGGAGCTGAAT |
| | | GAAGCCATACCAAACGACGAGCGTGACACCACGATGCCTGTAGCAATGG |
| | | CAACAACGTTGCGCAAACTATTAACTGGCGAACTACTTACTCTAGCTTCC |
| | | CGGCAACAATTAATAGACTGGATGGAGGCGGATAAAGTTGCAGGACCAC |
| | | TTCTGCGCTCGGCCCTTCCGGCTGGCTGGTTTATTGCTGATAAATCTGGAG |
| | | CCGGTGAGCGTGGGTCTCGCGGTATCATTGCAGCACTGGGGCCAGATGGT |
| | | AAGCCCTCCCGTATCGTAGTTATCTACACGACGGGGAGTCAGGCAACTAT |
| | | GGATGAACGAAATAGACAGATCGCTGAGATAGGTGCCTCACTGATTAAG |
| | | CATTGGTAACTGTCAGACCAAGTTTACTCATATATACTTTAGATTGATTTA |
| | | AAACTTCATTTTTAATTTAAAAGGATCTAGGTGAAGATCCTTTTTGATAAT |
| | | CTCATGACCAAAATCCCTTAACGTGAGTTTTCGTTCCACTGAGCGTCAGA |
| | | CCCC |
| 156 | <#3118 | GTAGAAAAGATCAAAGGATCTTCTTGAGATCCTTTTTTTCTGCGCGTAATC |
| | pAAV_FOXP3. | TGCTGCTTGCAAACAAAAAAACCACCGCTACCAGCGGTGGTTTGTTTGCC |
| | 045_MND.LNG | GGATCAAGAGCTACCAACTCTTTTTCCGAAGGTAACTGGCTTCAGCAGAG |
| | FR-P2A- | CGCAGATACCAAATACTGTCCTTCTAGTGTAGCCGTAGTTAGGCCACCAC |
| | FOXP3geneartC | TTCAAGAACTCTGTAGCACCGCCTACATACCTCGCTCTGCTAATCCTGTTA |
| | DS.3UTR_045_f | CCAGTGGCTGCTGCCAGTGGCGATAAGTCGTGTCTTACCGGGTTGGACTC |
| | or T9> | AAGACGATAGTTACCGGATAAGGCGCAGCGGTCGGGCTGAACGGGGGGT |
| | | TCGTGCACACAGCCCAGCTTGGAGCGAACGACCTACACCGAACTGAGAT |
| | | ACCTACAGCGTGAGCTATGAGAAAGCGCCACGCTTCCCGAAGGGAGAAA |
| | | GGCGGACAGGTATCCGGTAAGCGGCAGGGTCGGAACAGGAGAGCGCACG |
| | | AGGGAGCTTCCAGGGGGAAACGCCTGGTATCTTTATAGTCCTGTCGGGTT |
| | | TCGCCACCTCTGACTTGAGCGTCGATTTTTGTGATGCTCGTCAGGGGGGC |
| | | GGAGCCTATGGAAAAACGCCAGCAACGCGGCCTTTTTACGGTTCCTGGCC |
| | | TTTTGCTGGCCTTTTGCTCACATGTTCTTTCCTGCGTTATCCCCTGATTCTG |
| | | TGGATAACCGTATTACCGCCTTTGAGTGAGCTGATACCGCTCGCCGCAGC |
| | | CGAACGACCGAGCGCAGCGAGTCAGTGAGCGAGGAAGCGGAAGAGCGC |
| | | CCAATACGCAAACCGCCTCTCCCCGCGCGTTGGCCGATTCATTAATGCAG |
| | | CTGCGCGCTCGCTCGCTCACTGAGGCCGCCCGGGCAAAGCCCGGGCGTCG |
| | | GGCGACCTTTGGTCGCCCGGCCTCAGTGAGCGAGCGAGCGCGCAGAGAG |
| | | GGAGTGGCCAACTCCATCACTAGGGGTTCCTTGTAGTTAATGATTAACCC |
| | | GCCATGCTACTTATCTACGTAGCGGCCGCAGCCTGTGCAGGGTGCAGGGA |
| | | GGGCTAGAGGCCTGAGGCTTGAAACAGCTCTCAAGTGGAGGGGGAAACA |
| | | ACCATTGCCCTCATAGAGGACACATCCACACCAGGGCTGTGCTAGCGTGG |
| | | GCAGGCAAGCCAGGTGCTGGACCTCTGCACGTGGGGCATGTGTGGGTAT |
| | | GTACATGTACCTGTGTTCTTGGTGTGTGTGTGTGTGTGTGTGTGTGTGTGT |
| | | GTCTAGAGCTGGGGTGCAACTATGGGGCCCCTCGGGACATGTCCCAGCCA |
| | | ATGCCTGCTTTGACCAGAGGAGTGTCCACGTGGCTCAGGTGGTCGAGTAT |
| | | CTCATACCGCCCTAGCACACGTGTGACTCCTTTCCCCTATTGTCTACGCAG |
| | | CCTGCCCTTGGACAAGGACCCGATGCCCAACCCCAGGCCTGGCAAGCCCT |
| | | CGGCCCCTTCCTTGGCCCTTGGCCCATCCCCACGCGTAGGAACAGAGAAA |
| | | CAGGAGAATATGGGCCAAACAGGATATCTGTGGTAAGCAGTTCCTGCCCC |
| | | GGCTCAGGGCCAAGAACAGTTGGAACAGCAGAATATGGGCCAAACAGGA |
| | | TATCTGTGGTAAGCAGTTCCTGCCCCGGCTCAGGGCCAAGAACAGATGGT |
| | | CCCCAGATGCGGTCCCGCCCTCAGCAGTTTCTAGAGAACCATCAGATGTT |
| | | TCCAGGGTGCCCCAAGGACCTGAAATGACCCTGTGCCTTATTTGAACTAA |
| | | CCAATCAGTTCGCTTCTCGCTTCTGTTCGCGCGCTTCTGCTCCCCGAGCTC |
| | | TATATAAGCAGAGCTCGTTTAGTGAACCGTCAGATCGCCTGGAGACGCCA |
| | | TCCACGCTGTTTTGACTTCCATAGAAGGATCTCGAGGCCACCATGGGGGC |
| | | AGGTGCCACCGGACGAGCCATGGACGGGCCGCGCCTGCTGCTGTTGCTGC |
| | | TTCTGGGGGTGTCCCTTGGAGGTGCCAAGGAGGCATGCCCCACAGGCCTG |
| | | TACACACACAGCGGTGAGTGCTGCAAAGCCTGCAACCTGGGCGAGGGTG |
| | | TGGCCCAGCCTTGTGGAGCCAACCAGACCGTGTGTGAGCCCTGCCTGGAC |
| | | AGCGTGACGTTCTCCGACGTGGTGAGCGCGACCGAGCCGTGCAAGCCGT |
| | | GCACCGAGTGCGTGGGGCTCCAGAGCATGTCGGCGCCGTGCGTGGAGGC |
| | | CGACGACGCCGTGTGCCGCTGCGCCTACGGCTACTACCAGGATGAGACG |
| | | ACTGGGCGCTGCGAGGCGTGCCGCGTGTGCGAGGCGGGCTCGGGCCTCG |
| | | TGTTCTCCTGCCAGGACAAGCAGAACACCGTGTGCGAGGAGTGCCCCGAC |
| | | GGCACGTATTCCGACGAGGCCAACCACGTGGACCCGTGCCTGCCCTGCAC |
| | | CGTGTGCGAGGACACCGAGCGCCAGCTCCGCGAGTGCACACGCTGGGCC |
| | | GACGCCGAGTGCGAGGAGATCCCTGGCCGTTGGATTACACGGTCCACACC |
| | | CCCAGAGGGCTCGGACAGCACAGCCCCCAGCACCCAGGAGCCTGAGGCA |
| | | CCTCCAGAACAAGACCTCATAGCCAGCACGGTGGCAGGTGTGGTGACCA |
| | | CAGTGATGGGCAGCTCCCAGCCCGTGGTGACCCGAGGCACCACCGACAA |
| | | CCTCATCCCTGTCTATTGCTCCATCCTGGCTGCTGTGGTTGTGGGTCTTGT |
| | | GGCCTACATAGCCTTCAAGAGGGGAAGCGGAGCGACTAACTTCAGCCTG |
| | | CTGAAGCAGGCCGGAGATGTGGAGGAAAACCCTGGACCGATGCCTAATC |
| | | CTCGGCCTGGAAAGCCTAGCGCTCCTTCTCTTGCTCTGGGACCTTCTCCTG |
| | | GCGCCTCTCCATCTTGGAGAGCCGCTCCTAAAGCCAGCGATCTGCTGGGA |
| | | GCTAGAGGACCTGGCGGCACATTTCAGGGCAGAGATCTTAGAGGCGGAG |
| | | CCCACGCTAGCTCCTCCAGCCTTAATCCTATGCCTCCTAGCCAGCTCCAGC |
| | | TGCCTACACTGCCTCTGGTTATGGTGGCTCCTAGCGGAGCTAGACTGGGC |
| | | CCTCTGCCTCATCTGCAAGCTCTGCTGCAGGACAGACCCCACTTCATGCA |
| | | CCAGCTGAGCACCGTGGATGCCCACGCAAGAACACCTGTGCTGCAGGTTC |
| | | ACCCTCTGGAATCCCCAGCCATGATCAGCCTGACACCTCCAACAACAGCC |
| | | ACCGGCGTGTTCAGCCTGAAAGCCAGACCTGGACTGCCTCCTGGCATCAA |
| | | TGTGGCCAGCCTGGAATGGGTGTCCAGAGAACCTGCTCTGCTGTGCACAT |
| | | TCCCCAATCCAAGCGCTCCCAGAAAGGACAGCACACTGTCTGCCGTGCCT |
| | | CAGAGCAGCTATCCCCTGCTTGCTAACGGCGTGTGCAAGTGGCCTGGATG |
| | | CGAGAAGGTGTTCGAGGAACCCGAGGACTTCCTGAAGCACTGCCAGGCC |
| | | GATCATCTGCTGGACGAGAAAGGCAGAGCCCAGTGTCTGCTCCAGCGCG |
| | | AGATGGTGCAGTCTCTGGAACAGCAGCTGGTCCTGGAAAAAGAAAAGCT |
| | | GAGCGCCATGCAGGCCCACCTGGCCGGAAAAATGGCCCTGACAAAGGCC |
| | | AGCAGCGTGGCCTCTTCTGATAAGGGCAGCTGCTGCATTGTGGCCGCTGG |
| | | ATCTCAGGGACCTGTGGTTCCTGCTTGGAGCGGACCTAGAGAGGCCCCTG |
| | | ATTCTCTGTTTGCCGTGCGGAGACACCTGTGGGGCTCTCACGGCAACTCT |
| | | ACTTTCCCCGAGTTCCTGCACAACATGGACTACTTCAAGTTCCACAACAT |
| | | GCGGCCTCCATTCACCTACGCCACACTGATCAGATGGGCCATTCTGGAAG |
| | | CCCCTGAGAAGCAGAGAACCCTGAACGAGATCTACCACTGGTTTACCCGG |
| | | ATGTTCGCCTTCTTCCGGAATCACCCTGCCACCTGGAAGAACGCCATCCG |
| | | GCACAATCTGAGCCTGCACAAGTGCTTCGTGCGCGTGGAATCTGAGAAAG |
| | | GCGCCGTGTGGACAGTGGACGAGCTGGAATTCAGAAAGAAGAGAAGCCA |
| | | GCGGCCTAGCCGGTGCAGCAATCCTACACCTGGACCTTGAAAGCTTGTCG |
| | | ACCCTCAAGATCAAGGAAAGGAGGATGGACGAACAGGGGCCAAACTGGT |
| | | GGGAGGCAGAGGTGGTGGGGGCAGGGATGATAGGCCCTGGATGTGCCCA |
| | | CAGGGACCAAGAAGTGAGGTTTCCACTGTCTTGCCTGCCAGGGCCCCTGT |
| | | TCCCCCGCTGGCAGCCACCCCCTCCCCCATCATATCCTTTGCCCCAAGGCT |
| | | GCTCAGAGGGGCCCCGGTCCTGGCCCCAGCCCCCACCTCCGCCCCAGACA |
| | | CACCCCCCAGTCGAGCCCTGCAGCCAAACAGAGCCTTCACAACCAGCCAC |
| | | ACAGAGCCTGCCTCAGCTGCTCGCACAGATTACTTCAGGGCTGGAAAAGT |
| | | CACACAGACACACAAAATGTCACAATCCTGTCCCTCACTCAACACAAACC |
| | | CCAAAACACAGAGAGCCTGCCTCAGTACACTCAAACAACCTCAAAGCTG |
| | | CATCATCACACAATCACACACAAGCACAGCCCTGACAACCCACACACCCC |
| | | AAGGCACGCACCCACAGCCAGCCTCAGGGCCCACAGGGGCACTGTCAAC |
| | | ACAGGGGTGTGCCCAGAGGCCTACACAGAAGCAGCGTCAGTACCCTCAG |
| | | GATCTGAGGTCCCAACACGTGCTCGCTCACACACACGGCCTGTTAGAATT |
| | | CACCTGTGTATCTCACGCATATGCACACGCACAGCCCCCCAGTGGGTCTC |
| | | TTGAGTCCCGTGCAGACACACACAGCCACACACACTGCCTTGCCAAAAAT |
| | | ACCCCGTGTCTCCCCTGCCACTCACCTCACTCCCATTCCCTGAGCCCTGAT |
| | | CCATGCCTCAGCTTAGACTGCAGAGGAACTACTCATTTATTTGGGATCCA |
| | | AGGCCCCCAACCCACAGTACCGTCCCCAATAAACTGCAGCCGAGCTCCCC |
| | | ACAACTAGTGCCTCGCCCAGCTGGAGGGCTGCACCCAAAGCCTCAGACCT |
| | | GCTGGGGGCCCGGGGCCCAGGGGGAACCTTCCAGGGCCGAGATCTTCGA |
| | | GGCGGGGCCCATGCCTCCTCTTCTTCCTTGAACCCCATGCCACCATCGCA |
| | | GCTGCAGGTGAGGCCCTGGGCCCAGGATGGGGCAGGCAGGGTGGGGTAC |
| | | CTGGACCTACAGGTGCCGACCTTTACTGTGGCACTGGGCGGGAGGGGGG |
| | | CTGGCTGGGGCACAGGAAGTGGTTTCTGGGTCCCAGGCAAGTCTGTGACT |
| | | TATGCAGATGTTGCAGGGCCAAGAAAATCCCCACCTGCCAGGCCTCAGA |
| | | GATTGGAGGCTCTCCCCGACCTCCCAATCCCTGTCTCAGGAGAGGAGGAG |
| | | GCCGTATTGTAGTCCCATGAGCATAGCTATGTGTCCCCATCCCCATGTGA |
| | | CAAGAGAAGAGGAGGATCCTACGTAGATAAGTAGCATGGCGGGTTAATC |
| | | ATTAACTACAAGGAACCCCTAGTGATGGAGTTGGCCACTCCCTCTCTGCG |
| | | CGCTCGCTCGCTCACTGAGGCCGGGCGACCAAAGGTCGCCCGACGCCCG |
| | | GGCTTTGCCCGGGCGGCCTCAGTGAGCGAGCGAGCGCGCCAGCTGGCGT |
| | | AATAGCGAAGAGGCCCGCACCGATCGCCCTTCCCAACAGTTGCGCAGCCT |
| | | GAATGGCGAATGGCGATTCCGTTGCAATGGCTGGCGGTAATATTGTTCTG |
| | | GATATTACCAGCAAGGCCGATAGTTTGAGTTCTTCTACTCAGGCAAGTGA |
| | | TGTTATTACTAATCAAAGAAGTATTGCGACAACGGTTAATTTGCGTGATG |
| | | GACAGACTCTTTTACTCGGTGGCCTCACTGATTATAAAAACACTTCTCAG |
| | | GATTCTGGCGTACCGTTCCTGTCTAAAATCCCTTTAATCGGCCTCCTGTTT |
| | | AGCTCCCGCTCTGATTCTAACGAGGAAAGCACGTTATACGTGCTCGTCAA |
| | | AGCAACCATAGTACGCGCCCTGTAGCGGCGCATTAAGCGCGGCGGGTGT |
| | | GGTGGTTACGCGCAGCGTGACCGCTACACTTGCCAGCGCCCTAGCGCCCG |
| | | CTCCTTTCGCTTTCTTCCCTTCCTTTCTCGCCACGTTCGCCGGCTTTCCCCG |
| | | TCAAGCTCTAAATCGGGGGCTCCCTTTAGGGTTCCGATTTAGTGCTTTACG |
| | | GCACCTCGACCCCAAAAAACTTGATTAGGGTGATGGTTCACGTAGTGGGC |
| | | CATCGCCCTGATAGACGGTTTTTCGCCCTTTGACGTTGGAGTCCACGTTCT |
| | | TTAATAGTGGACTCTTGTTCCAAACTGGAACAACACTCAACCCTATCTCG |
| | | GTCTATTCTTTTGATTTATAAGGGATTTTGCCGATTTCGGCCTATTGGTTA |
| | | AAAAATGAGCTGATTTAACAAAAATTTAACGCGAATTTTAACAAAATATT |
| | | AACGTTTACAATTTAAATATTTGCTTATACAATCTTCCTGTTTTTGGGGCT |
| | | TTTCTGATTATCAACCGGGGTACATATGATTGACATGCTAGTTTTACGATT |
| | | ACCGTTCATCGATTCTCTTGTTTGCTCCAGACTCTCAGGCAATGACCTGAT |
| | | AGCCTTTGTAGAGACCTCTCAAAAATAGCTACCCTCTCCGGCATGAATTT |
| | | ATCAGCTAGAACGGTTGAATATCATATTGATGGTGATTTGACTGTCTCCG |
| | | GCCTTTCTCACCCGTTTGAATCTTTACCTACACATTACTCAGGCATTGCAT |
| | | TTAAAATATATGAGGGTTCTAAAAATTTTTATCCTTGCGTTGAAATAAAG |
| | | GCTTCTCCCGCAAAAGTATTACAGGGTCATAATGTTTTTGGTACAACCGA |
| | | TTTAGCTTTATGCTCTGAGGCTTTATTGCTTAATTTTGCTAATTCTTTGCCT |
| | | TGCCTGTATGATTTATTGGATGTTGGAATCGCCTGATGCGGTATTTTCTCC |
| | | TTACGCATCTGTGCGGTATTTCACACCGCATATGGTGCACTCTCAGTACA |
| | | ATCTGCTCTGATGCCGCATAGTTAAGCCAGCCCCGACACCCGCCAACACC |
| | | CGCTGACGCGCCCTGACGGGCTTGTCTGCTCCCGGCATCCGCTTACAGAC |
| | | AAGCTGTGACCGTCTCCGGGAGCTGCATGTGTCAGAGGTTTTCACCGTCA |
| | | TCACCGAAACGCGCGAGACGAAAGGGCCTCGTGATACGCCTATTTTTATA |
| | | GGTTAATGTCATGATAATAATGGTTTCTTAGACGTCAGGTGGCACTTTTCG |
| | | GGGAAATGTGCGCGGAACCCCTATTTGTTTATTTTTCTAAATACATTCAAA |
| | | TATGTATCCGCTCATGAGACAATAACCCTGATAAATGCTTCAATAATATT |
| | | GAAAAAGGAAGAGTATGAGTATTCAACATTTCCGTGTCGCCCTTATTCCC |
| | | TTTTTTGCGGCATTTTGCCTTCCTGTTTTTGCTCACCCAGAAACGCTGGTG |
| | | AAAGTAAAAGATGCTGAAGATCAGTTGGGTGCACGAGTGGGTTACATCG |
| | | AACTGGATCTCAACAGCGGTAAGATCCTTGAGAGTTTTCGCCCCGAAGAA |
| | | CGTTTTCCAATGATGAGCACTTTTAAAGTTCTGCTATGTGGCGCGGTATTA |
| | | TCCCGTATTGACGCCGGGCAAGAGCAACTCGGTCGCCGCATACACTATTC |
| | | TCAGAATGACTTGGTTGAGTACTCACCAGTCACAGAAAAGCATCTTACGG |
| | | ATGGCATGACAGTAAGAGAATTATGCAGTGCTGCCATAACCATGAGTGAT |
| | | AACACTGCGGCCAACTTACTTCTGACAACGATCGGAGGACCGAAGGAGC |
| | | TAACCGCTTTTTTGCACAACATGGGGGATCATGTAACTCGCCTTGATCGTT |
| | | GGGAACCGGAGCTGAATGAAGCCATACCAAACGACGAGCGTGACACCAC |
| | | GATGCCTGTAGCAATGGCAACAACGTTGCGCAAACTATTAACTGGCGAAC |
| | | TACTTACTCTAGCTTCCCGGCAACAATTAATAGACTGGATGGAGGCGGAT |
| | | AAAGTTGCAGGACCACTTCTGCGCTCGGCCCTTCCGGCTGGCTGGTTTATT |
| | | GCTGATAAATCTGGAGCCGGTGAGCGTGGGTCTCGCGGTATCATTGCAGC |
| | | ACTGGGGCCAGATGGTAAGCCCTCCCGTATCGTAGTTATCTACACGACGG |
| | | GGAGTCAGGCAACTATGGATGAACGAAATAGACAGATCGCTGAGATAGG |
| | | TGCCTCACTGATTAAGCATTGGTAACTGTCAGACCAAGTTTACTCATATAT |
| | | ACTTTAGATTGATTTAAAACTTCATTTTTAATTTAAAAGGATCTAGGTGAA |
| | | GATCCTTTTTGATAATCTCATGACCAAAATCCCTTAACGTGAGTTTTCGTT |
| | | CCACTGAGCGTCAGACCCC |
| 157 | <#1390 pAAV- | GTAGAAAAGATCAAAGGATCTTCTTGAGATCCTTTTTTTCTGCGCGTAATC |
| | FOXP3_0.9[MN | TGCTGCTTGCAAACAAAAAAACCACCGCTACCAGCGGTGGTTTGTTTGCC |
| | D-GFPki]_0.9 | GGATCAAGAGCTACCAACTCTTTTTCCGAAGGTAACTGGCTTCAGCAGAG |
| | (noUCOEctr1)> | CGCAGATACCAAATACTGTCCTTCTAGTGTAGCCGTAGTTAGGCCACCAC |
| | | TTCAAGAACTCTGTAGCACCGCCTACATACCTCGCTCTGCTAATCCTGTTA |
| | | CCAGTGGCTGCTGCCAGTGGCGATAAGTCGTGTCTTACCGGGTTGGACTC |
| | | AAGACGATAGTTACCGGATAAGGCGCAGCGGTCGGGCTGAACGGGGGGT |
| | | TCGTGCACACAGCCCAGCTTGGAGCGAACGACCTACACCGAACTGAGAT |
| | | ACCTACAGCGTGAGCTATGAGAAAGCGCCACGCTTCCCGAAGGGAGAAA |
| | | GGCGGACAGGTATCCGGTAAGCGGCAGGGTCGGAACAGGAGAGCGCACG |
| | | AGGGAGCTTCCAGGGGGAAACGCCTGGTATCTTTATAGTCCTGTCGGGTT |
| | | TCGCCACCTCTGACTTGAGCGTCGATTTTTGTGATGCTCGTCAGGGGGGC |
| | | GGAGCCTATGGAAAAACGCCAGCAACGCGGCCTTTTTACGGTTCCTGGCC |
| | | TTTTGCTGGCCTTTTGCTCACATGTTCTTTCCTGCGTTATCCCCTGATTCTG |
| | | TGGATAACCGTATTACCGCCTTTGAGTGAGCTGATACCGCTCGCCGCAGC |
| | | CGAACGACCGAGCGCAGCGAGTCAGTGAGCGAGGAAGCGGAAGAGCGC |
| | | CCAATACGCAAACCGCCTCTCCCCGCGCGTTGGCCGATTCATTAATGCAG |
| | | CTGCGCGCTCGCTCGCTCACTGAGGCCGCCCGGGCAAAGCCCGGGCGTCG |
| | | GGCGACCTTTGGTCGCCCGGCCTCAGTGAGCGAGCGAGCGCGCAGAGAG |
| | | GGAGTGGCCAACTCCATCACTAGGGGTTCCTTGTAGTTAATGATTAACCC |
| | | GCCATGCTACTTATCTACGTAGCGGCCGCGCTCAAGAGACCCCATCTCTC |
| | | CTCCTCTCTGTCACTTGCCATGCTGGATCCGTGCATGATCACACTCCTGGA |
| | | CTCGCCTCCTTGCCCTGAGATCCAGACCCCCGTATTCAGCTGCCCCCTCAG |
| | | CTCCTCCACTCACATATTTAATGCCAGACTCTTCATGTCTATCTACACCTG |
| | | CACTTTTGCACCCAATCCAACTCCCCGCCATGTCCCCCATCTCAGGTAATG |
| | | TCAGCTCGGTCCTTCCAGCTGCTCAAGCTAAAACCCATGTCACTTTGACTC |
| | | TCCCTCTTGCCCACTACATCCAAGCTGCTAGCACTGCTCCTGATCCAGCTT |
| | | CAGATTAAGTCTCAGAATCTACCCACTTCTCGCCTTCTCCACTGCCACCAG |
| | | CCCATTCTGTGCCAGCATCATCACTTGCCAGGACTGTTACAATAGCCTCCT |
| | | CACTAGCCCCACTCACAGCAGCCAGATGAATCTTTTGAGTCCATGCCTAG |
| | | TCACTGGGGCAAAATAGGACTCCGAGGAGAAAGTCCGAGACCAGCTCCG |
| | | GCAAGATGAGCAAACACAGCCTGTGCAGGGTGCAGGGAGGGCTAGAGGC |
| | | CTGAGGCTTGAAACAGCTCTCAAGTGGAGGGGGAAACAACCATTGCCCT |
| | | CATAGAGGACACATCCACACCAGGGCTGTGCTAGCGTGGGCAGGCAAGC |
| | | CAGGTGCTGGACCTCTGCACGTGGGGCATGTGTGGGTATGTACATGTACC |
| | | TGTGTTCTTGGTGTGTGTGTGTGTGTGTGTGTGTGTGTGTGTCTAGAGCTG |
| | | GGGTGCAACTATGGGGCCCCTCGGGACATGTCCCAGCCAATGCCTGCTTT |
| | | GACCAGAGGAGTGTCCACGTGGCTCAGGTGGTCGAGTATCTCATACCGCC |
| | | CTAGCACACGTGTGACTCCTTTCCCCTATTGTCTACCCCGGGGAACAGAG |
| | | AAACAGGAGAATATGGGCCAAACAGGATATCTGTGGTAAGCAGTTCCTG |
| | | CCCCGGCTCAGGGCCAAGAACAGTTGGAACAGCAGAATATGGGCCAAAC |
| | | AGGATATCTGTGGTAAGCAGTTCCTGCCCCGGCTCAGGGCCAAGAACAG |
| | | ATGGTCCCCAGATGCGGTCCCGCCCTCAGCAGTTTCTAGAGAACCATCAG |
| | | ATGTTTCCAGGGTGCCCCAAGGACCTGAAATGACCCTGTGCCTTATTTGA |
| | | ACTAACCAATCAGTTCGCTTCTCGCTTCTGTTCGCGCGCTTCTGCTCCCCG |
| | | AGCTCTATATAAGCAGAGCTCGTTTAGTGAACCGTCAGATCGTCTACGCA |
| | | GCCTGCCCTTGGACAAGGACCCGATGCCCAACCCCAGGCCTGTGAGCAA |
| | | GGGCGAGGAGCTGTTCACCGGGGTGGTGCCCATCCTGGTCGAGCTGGAC |
| | | GGCGACGTAAACGGCCACAAGTTCAGCGTGTCCGGCGAGGGCGAGGGCG |
| | | ATGCCACCTACGGCAAGCTGACCCTGAAGTTCATCTGCACCACCGGCAAG |
| | | CTGCCCGTGCCCTGGCCCACCCTCGTGACCACCCTGACCTACGGCGTGCA |
| | | GTGCTTCAGCCGCTACCCCGACCACATGAAGCAGCACGACTTCTTCAAGT |
| | | CCGCCATGCCCGAAGGCTACGTCCAGGAGCGCACCATCTTCTTCAAGGAC |
| | | GACGGCAACTACAAGACCCGCGCCGAGGTGAAGTTCGAGGGCGACACCC |
| | | TGGTGAACCGCATCGAGCTGAAGGGCATCGACTTCAAGGAGGACGGCAA |
| | | CATCCTGGGGCACAAGCTGGAGTACAACTACAACAGCCACAACGTCTAT |
| | | ATCATGGCCGACAAGCAGAAGAACGGCATCAAGGTGAACTTCAAGATCC |
| | | GCCACAACATCGAGGACGGCAGCGTGCAGCTCGCCGACCACTACCAGCA |
| | | GAACACCCCCATCGGCGACGGCCCCGTGCTGCTGCCCGACAACCACTACC |
| | | TGAGCACCCAGTCCGCCCTGAGCAAAGACCCCAACGAGAAGCGCGATCA |
| | | CATGGTCCTGCTGGAGTTCGTGACCGCCGCCGGGATCACTCTCGGCATGG |
| | | ACGAGCTGTACAAGGGCAAGCCCTCGGCCCCTTCCTTGGCCCTTGGCCCA |
| | | TCCCCAGGAGCCTCGCCCAGCTGGAGGGCTGCCCCTAAAGCAAGCGACCT |
| | | GCTGGGGGCCCGGGGCCCGGGTGGCACGTTCCAGGGCCGAGATCTTCGA |
| | | GGCGGGGCCCATGCCTCCTCTTCTTCCTTGAACCCCATGCCACCATCGCA |
| | | GCTGCAGGTGAGGCCCTGGGCCCAGGATGGGGCAGGCAGGGTGGGGTAC |
| | | CTGGACCTACAGGTGCCGACCTTTACTGTGGCACTGGGCGGGAGGGGGG |
| | | CTGGCTGGGGCACAGGAAGTGGTTTCTGGGTCCCAGGCAAGTCTGTGACT |
| | | TATGCAGATGTTGCAGGGCCAAGAAAATCCCCACCTGCCAGGCCTCAGA |
| | | GATTGGAGGCTCTCCCCGACCTCCCAATCCCTGTCTCAGGAGAGGAGGAG |
| | | GCCGTATTGTAGTCCCATGAGCATAGCTATGTGTCCCCATCCCCATGTGA |
| | | CAAGAGAAGAGGACTGGGGCCAAGTAGGTGAGGTGACAGGGCTGAGGC |
| | | CAGCTCTGCAACTTATTAGCTGTTTGATCTTTAAAAAGTTACTCGATCTCC |
| | | ATGAGCCTCAGTTTCCATACGTGTAAAAGGGGGATGATCATAGCATCTAC |
| | | CATGTGGGCTTGCAGTGCAGAGTATTTGAATTAGACACAGAACAGTGAG |
| | | GATCAGGATGGCCTCTCACCCACCTGCCTTTCTGCCCAGCTGCCCACACT |
| | | GCCCCTAGTCATGGTGGCACCCTCCGGGGCACGGCTGGGCCCCTTGCCCC |
| | | ACTTACAGGCACTCCTCCAGGACAGGCCACATTTCATGCACCAGGTATGG |
| | | ACGGTGAATGGGCAGGGAGGAGGGAGCAGGTGGGAGAACTGTGGGGAG |
| | | GGGCCCCGAGTCAGGCTGAACCACAGCCCACATGGCGGCCGCTACGTAG |
| | | ATAAGTAGCATGGCGGGTTAATCATTAACTACAAGGAACCCCTAGTGATG |
| | | GAGTTGGCCACTCCCTCTCTGCGCGCTCGCTCGCTCACTGAGGCCGGGCG |
| | | ACCAAAGGTCGCCCGACGCCCGGGCTTTGCCCGGGCGGCCTCAGTGAGC |
| | | GAGCGAGCGCGCCAGCTGGCGTAATAGCGAAGAGGCCCGCACCGATCGC |
| | | CCTTCCCAACAGTTGCGCAGCCTGAATGGCGAATGGCGATTCCGTTGCAA |
| | | TGGCTGGCGGTAATATTGTTCTGGATATTACCAGCAAGGCCGATAGTTTG |
| | | AGTTCTTCTACTCAGGCAAGTGATGTTATTACTAATCAAAGAAGTATTGC |
| | | GACAACGGTTAATTTGCGTGATGGACAGACTCTTTTACTCGGTGGCCTCA |
| | | CTGATTATAAAAACACTTCTCAGGATTCTGGCGTACCGTTCCTGTCTAAA |
| | | ATCCCTTTAATCGGCCTCCTGTTTAGCTCCCGCTCTGATTCTAACGAGGAA |
| | | AGCACGTTATACGTGCTCGTCAAAGCAACCATAGTACGCGCCCTGTAGCG |
| | | GCGCATTAAGCGCGGCGGGTGTGGTGGTTACGCGCAGCGTGACCGCTAC |
| | | ACTTGCCAGCGCCCTAGCGCCCGCTCCTTTCGCTTTCTTCCCTTCCTTTCTC |
| | | GCCACGTTCGCCGGCTTTCCCCGTCAAGCTCTAAATCGGGGGCTCCCTTTA |
| | | GGGTTCCGATTTAGTGCTTTACGGCACCTCGACCCCAAAAAACTTGATTA |
| | | GGGTGATGGTTCACGTAGTGGGCCATCGCCCTGATAGACGGTTTTTCGCC |
| | | CTTTGACGTTGGAGTCCACGTTCTTTAATAGTGGACTCTTGTTCCAAACTG |
| | | GAACAACACTCAACCCTATCTCGGTCTATTCTTTTGATTTATAAGGGATTT |
| | | TGCCGATTTCGGCCTATTGGTTAAAAAATGAGCTGATTTAACAAAAATTT |
| | | AACGCGAATTTTAACAAAATATTAACGTTTACAATTTAAATATTTGCTTAT |
| | | ACAATCTTCCTGTTTTTGGGGCTTTTCTGATTATCAACCGGGGTACATATG |
| | | ATTGACATGCTAGTTTTACGATTACCGTTCATCGATTCTCTTGTTTGCTCC |
| | | AGACTCTCAGGCAATGACCTGATAGCCTTTGTAGAGACCTCTCAAAAATA |
| | | GCTACCCTCTCCGGCATGAATTTATCAGCTAGAACGGTTGAATATCATAT |
| | | TGATGGTGATTTGACTGTCTCCGGCCTTTCTCACCCGTTTGAATCTTTACC |
| | | TACACATTACTCAGGCATTGCATTTAAAATATATGAGGGTTCTAAAAATT |
| | | TTTATCCTTGCGTTGAAATAAAGGCTTCTCCCGCAAAAGTATTACAGGGT |
| | | CATAATGTTTTTGGTACAACCGATTTAGCTTTATGCTCTGAGGCTTTATTG |
| | | CTTAATTTTGCTAATTCTTTGCCTTGCCTGTATGATTTATTGGATGTTGGA |
| | | ATCGCCTGATGCGGTATTTTCTCCTTACGCATCTGTGCGGTATTTCACACC |
| | | GCATATGGTGCACTCTCAGTACAATCTGCTCTGATGCCGCATAGTTAAGC |
| | | CAGCCCCGACACCCGCCAACACCCGCTGACGCGCCCTGACGGGCTTGTCT |
| | | GCTCCCGGCATCCGCTTACAGACAAGCTGTGACCGTCTCCGGGAGCTGCA |
| | | TGTGTCAGAGGTTTTCACCGTCATCACCGAAACGCGCGAGACGAAAGGG |
| | | CCTCGTGATACGCCTATTTTTATAGGTTAATGTCATGATAATAATGGTTTC |
| | | TTAGACGTCAGGTGGCACTTTTCGGGGAAATGTGCGCGGAACCCCTATTT |
| | | GTTTATTTTTCTAAATACATTCAAATATGTATCCGCTCATGAGACAATAAC |
| | | CCTGATAAATGCTTCAATAATATTGAAAAAGGAAGAGTATGAGTATTCAA |
| | | CATTTCCGTGTCGCCCTTATTCCCTTTTTTGCGGCATTTTGCCTTCCTGTTT |
| | | TTGCTCACCCAGAAACGCTGGTGAAAGTAAAAGATGCTGAAGATCAGTT |
| | | GGGTGCACGAGTGGGTTACATCGAACTGGATCTCAACAGCGGTAAGATC |
| | | CTTGAGAGTTTTCGCCCCGAAGAACGTTTTCCAATGATGAGCACTTTTAA |
| | | AGTTCTGCTATGTGGCGCGGTATTATCCCGTATTGACGCCGGGCAAGAGC |
| | | AACTCGGTCGCCGCATACACTATTCTCAGAATGACTTGGTTGAGTACTCA |
| | | CCAGTCACAGAAAAGCATCTTACGGATGGCATGACAGTAAGAGAATTAT |
| | | GCAGTGCTGCCATAACCATGAGTGATAACACTGCGGCCAACTTACTTCTG |
| | | ACAACGATCGGAGGACCGAAGGAGCTAACCGCTTTTTTGCACAACATGG |
| | | GGGATCATGTAACTCGCCTTGATCGTTGGGAACCGGAGCTGAATGAAGCC |
| | | ATACCAAACGACGAGCGTGACACCACGATGCCTGTAGCAATGGCAACAA |
| | | CGTTGCGCAAACTATTAACTGGCGAACTACTTACTCTAGCTTCCCGGCAA |
| | | CAATTAATAGACTGGATGGAGGCGGATAAAGTTGCAGGACCACTTCTGC |
| | | GCTCGGCCCTTCCGGCTGGCTGGTTTATTGCTGATAAATCTGGAGCCGGT |
| | | GAGCGTGGGTCTCGCGGTATCATTGCAGCACTGGGGCCAGATGGTAAGCC |
| | | CTCCCGTATCGTAGTTATCTACACGACGGGGAGTCAGGCAACTATGGATG |
| | | AACGAAATAGACAGATCGCTGAGATAGGTGCCTCACTGATTAAGCATTG |
| | | GTAACTGTCAGACCAAGTTTACTCATATATACTTTAGATTGATTTAAAACT |
| | | TCATTTTTAATTTAAAAGGATCTAGGTGAAGATCCTTTTTGATAATCTCAT |
| | | GACCAAAATCCCTTAACGTGAGTTTTCGTTCCACTGAGCGTCAGACCCC |
| 158 | #1391 pAAV- | GTAGAAAAGATCAAAGGATCTTCTTGAGATCCTTTTTTTCTGCGCGTAATC |
| | FOXP3_0.9[FW | TGCTGCTTGCAAACAAAAAAACCACCGCTACCAGCGGTGGTTTGTTTGCC |
| | D0.7UCOE- | GGATCAAGAGCTACCAACTCTTTTTCCGAAGGTAACTGGCTTCAGCAGAG |
| | MND- | CGCAGATACCAAATACTGTCCTTCTAGTGTAGCCGTAGTTAGGCCACCAC |
| | GFPki]_0.9 | TTCAAGAACTCTGTAGCACCGCCTACATACCTCGCTCTGCTAATCCTGTTA |
| | | CCAGTGGCTGCTGCCAGTGGCGATAAGTCGTGTCTTACCGGGTTGGACTC |
| | | AAGACGATAGTTACCGGATAAGGCGCAGCGGTCGGGCTGAACGGGGGGT |
| | | TCGTGCACACAGCCCAGCTTGGAGCGAACGACCTACACCGAACTGAGAT |
| | | ACCTACAGCGTGAGCTATGAGAAAGCGCCACGCTTCCCGAAGGGAGAAA |
| | | GGCGGACAGGTATCCGGTAAGCGGCAGGGTCGGAACAGGAGAGCGCACG |
| | | AGGGAGCTTCCAGGGGGAAACGCCTGGTATCTTTATAGTCCTGTCGGGTT |
| | | TCGCCACCTCTGACTTGAGCGTCGATTTTTGTGATGCTCGTCAGGGGGGC |
| | | GGAGCCTATGGAAAAACGCCAGCAACGCGGCCTTTTTACGGTTCCTGGCC |
| | | TTTTGCTGGCCTTTTGCTCACATGTTCTTTCCTGCGTTATCCCCTGATTCTG |
| | | TGGATAACCGTATTACCGCCTTTGAGTGAGCTGATACCGCTCGCCGCAGC |
| | | CGAACGACCGAGCGCAGCGAGTCAGTGAGCGAGGAAGCGGAAGAGCGC |
| | | CCAATACGCAAACCGCCTCTCCCCGCGCGTTGGCCGATTCATTAATGCAG |
| | | CTGCGCGCTCGCTCGCTCACTGAGGCCGCCCGGGCAAAGCCCGGGCGTCG |
| | | GGCGACCTTTGGTCGCCCGGCCTCAGTGAGCGAGCGAGCGCGCAGAGAG |
| | | GGAGTGGCCAACTCCATCACTAGGGGTTCCTTGTAGTTAATGATTAACCC |
| | | GCCATGCTACTTATCTACGTAGCGGCCGCGCTCAAGAGACCCCATCTCTC |
| | | CTCCTCTCTGTCACTTGCCATGCTGGATCCGTGCATGATCACACTCCTGGA |
| | | CTCGCCTCCTTGCCCTGAGATCCAGACCCCCGTATTCAGCTGCCCCCTCAG |
| | | CTCCTCCACTCACATATTTAATGCCAGACTCTTCATGTCTATCTACACCTG |
| | | CACTTTTGCACCCAATCCAACTCCCCGCCATGTCCCCCATCTCAGGTAATG |
| | | TCAGCTCGGTCCTTCCAGCTGCTCAAGCTAAAACCCATGTCACTTTGACTC |
| | | TCCCTCTTGCCCACTACATCCAAGCTGCTAGCACTGCTCCTGATCCAGCTT |
| | | CAGATTAAGTCTCAGAATCTACCCACTTCTCGCCTTCTCCACTGCCACCAG |
| | | CCCATTCTGTGCCAGCATCATCACTTGCCAGGACTGTTACAATAGCCTCCT |
| | | CACTAGCCCCACTCACAGCAGCCAGATGAATCTTTTGAGTCCATGCCTAG |
| | | TCACTGGGGCAAAATAGGACTCCGAGGAGAAAGTCCGAGACCAGCTCCG |
| | | GCAAGATGAGCAAACACAGCCTGTGCAGGGTGCAGGGAGGGCTAGAGGC |
| | | CTGAGGCTTGAAACAGCTCTCAAGTGGAGGGGGAAACAACCATTGCCCT |
| | | CATAGAGGACACATCCACACCAGGGCTGTGCTAGCGTGGGCAGGCAAGC |
| | | CAGGTGCTGGACCTCTGCACGTGGGGCATGTGTGGGTATGTACATGTACC |
| | | TGTGTTCTTGGTGTGTGTGTGTGTGTGTGTGTGTGTGTGTGTCTAGAGCTG |
| | | GGGTGCAACTATGGGGCCCCTCGGGACATGTCCCAGCCAATGCCTGCTTT |
| | | GACCAGAGGAGTGTCCACGTGGCTCAGGTGGTCGAGTATCTCATACCGCC |
| | | CTAGCACACGTGTGACTCCTTTCCCCTATTGTCTACGCAAACACCCGAATC |
| | | AACTTCTAGTCAAATTATTGTTCACGCCGCAATGACCCACCCCTGGCCCG |
| | | CGTCTGTGGAACTGACCCCTGGTGTACAGGAGAGTTCGCTGCTGAAAGTG |
| | | GTCCCAAAGGGGTACTAGTTTTTAAGCTCCCAACTCCCCCTCCCCCAGCG |
| | | TCTGGAGGATTCCACACCCTCGCACCGCAGGGGCGAGGAAGTGGGCGGA |
| | | GTCCGGTTTTGGCGCCAGCCGCTGAGGCTGCCAAGCAGAAAAGCCACCG |
| | | CTGAGGAGACTCCGGTCACTGTCCTCGCCCCGCCTCCCCCTTCCCTCCCCT |
| | | TGGGGACCACCGGGCGCCACGCCGCGAACGGTAAGTGCCGCGGTCGTCG |
| | | GCGCCTCCGCCCTCCCCCTAGGGCCCCAATTCCCAGCGGGCGCGGCGCGC |
| | | GGCCCCTCCCCCCGCCGGGCGCGCGCCCGCTGCCCCGCCCTTCGTGGCCG |
| | | CCCGGCGTGGGCGGTGCCACCCCTCCCCCCGGCGGCCCCGCGCGCAGCTC |
| | | CCGGCTCCCTCCCCCTTCGGATGTGGCTTGAGCTGTAGGCGCGGAGGGCC |
| | | GGAGACGCTGCAGACCCGCGACCCGGAGCAGCTCGGAGGCGGTGAAGTC |
| | | GGTGGCTTTCCTTCTCTCTAGCTCTCGCTCGCTGGTGGTGCTTCAGATGCC |
| | | ACACGCGAACAGAGAAACAGGAGAATATGGGCCAAACAGGATATCTGTG |
| | | GTAAGCAGTTCCTGCCCCGGCTCAGGGCCAAGAACAGTTGGAACAGCAG |
| | | AATATGGGCCAAACAGGATATCTGTGGTAAGCAGTTCCTGCCCCGGCTCA |
| | | GGGCCAAGAACAGATGGTCCCCAGATGCGGTCCCGCCCTCAGCAGTTTCT |
| | | AGAGAACCATCAGATGTTTCCAGGGTGCCCCAAGGACCTGAAATGACCCT |
| | | GTGCCTTATTTGAACTAACCAATCAGTTCGCTTCTCGCTTCTGTTCGCGCG |
| | | CTTCTGCTCCCCGAGCTCTATATAAGCAGAGCTCGTTTAGTGAACCGTCA |
| | | GATCGTCTACGCAGCCTGCCCTTGGACAAGGACCCGATGCCCAACCCCAG |
| | | GCCTGTGAGCAAGGGCGAGGAGCTGTTCACCGGGGTGGTGCCCATCCTG |
| | | GTCGAGCTGGACGGCGACGTAAACGGCCACAAGTTCAGCGTGTCCGGCG |
| | | AGGGCGAGGGCGATGCCACCTACGGCAAGCTGACCCTGAAGTTCATCTG |
| | | CACCACCGGCAAGCTGCCCGTGCCCTGGCCCACCCTCGTGACCACCCTGA |
| | | CCTACGGCGTGCAGTGCTTCAGCCGCTACCCCGACCACATGAAGCAGCAC |
| | | GACTTCTTCAAGTCCGCCATGCCCGAAGGCTACGTCCAGGAGCGCACCAT |
| | | CTTCTTCAAGGACGACGGCAACTACAAGACCCGCGCCGAGGTGAAGTTC |
| | | GAGGGCGACACCCTGGTGAACCGCATCGAGCTGAAGGGCATCGACTTCA |
| | | AGGAGGACGGCAACATCCTGGGGCACAAGCTGGAGTACAACTACAACAG |
| | | CCACAACGTCTATATCATGGCCGACAAGCAGAAGAACGGCATCAAGGTG |
| | | AACTTCAAGATCCGCCACAACATCGAGGACGGCAGCGTGCAGCTCGCCG |
| | | ACCACTACCAGCAGAACACCCCCATCGGCGACGGCCCCGTGCTGCTGCCC |
| | | GACAACCACTACCTGAGCACCCAGTCCGCCCTGAGCAAAGACCCCAACG |
| | | AGAAGCGCGATCACATGGTCCTGCTGGAGTTCGTGACCGCCGCCGGGATC |
| | | ACTCTCGGCATGGACGAGCTGTACAAGGGCAAGCCCTCGGCCCCTTCCTT |
| | | GGCCCTTGGCCCATCCCCAGGAGCCTCGCCCAGCTGGAGGGCTGCCCCTA |
| | | AAGCAAGCGACCTGCTGGGGGCCCGGGGCCCGGGTGGCACGTTCCAGGG |
| | | CCGAGATCTTCGAGGCGGGGCCCATGCCTCCTCTTCTTCCTTGAACCCCAT |
| | | GCCACCATCGCAGCTGCAGGTGAGGCCCTGGGCCCAGGATGGGGCAGGC |
| | | AGGGTGGGGTACCTGGACCTACAGGTGCCGACCTTTACTGTGGCACTGGG |
| | | CGGGAGGGGGGCTGGCTGGGGCACAGGAAGTGGTTTCTGGGTCCCAGGC |
| | | AAGTCTGTGACTTATGCAGATGTTGCAGGGCCAAGAAAATCCCCACCTGC |
| | | CAGGCCTCAGAGATTGGAGGCTCTCCCCGACCTCCCAATCCCTGTCTCAG |
| | | GAGAGGAGGAGGCCGTATTGTAGTCCCATGAGCATAGCTATGTGTCCCCA |
| | | TCCCCATGTGACAAGAGAAGAGGACTGGGGCCAAGTAGGTGAGGTGACA |
| | | GGGCTGAGGCCAGCTCTGCAACTTATTAGCTGTTTGATCTTTAAAAAGTT |
| | | ACTCGATCTCCATGAGCCTCAGTTTCCATACGTGTAAAAGGGGGATGATC |
| | | ATAGCATCTACCATGTGGGCTTGCAGTGCAGAGTATTTGAATTAGACACA |
| | | GAACAGTGAGGATCAGGATGGCCTCTCACCCACCTGCCTTTCTGCCCAGC |
| | | TGCCCACACTGCCCCTAGTCATGGTGGCACCCTCCGGGGCACGGCTGGGC |
| | | CCCTTGCCCCACTTACAGGCACTCCTCCAGGACAGGCCACATTTCATGCA |
| | | CCAGGTATGGACGGTGAATGGGCAGGGAGGAGGGAGCAGGTGGGAGAA |
| | | CTGTGGGGAGGGGCCCCGAGTCAGGCTGAACCACAGCCCACATGGCGGC |
| | | CGCTACGTAGATAAGTAGCATGGCGGGTTAATCATTAACTACAAGGAACC |
| | | CCTAGTGATGGAGTTGGCCACTCCCTCTCTGCGCGCTCGCTCGCTCACTGA |
| | | GGCCGGGCGACCAAAGGTCGCCCGACGCCCGGGCTTTGCCCGGGCGGCC |
| | | TCAGTGAGCGAGCGAGCGCGCCAGCTGGCGTAATAGCGAAGAGGCCCGC |
| | | ACCGATCGCCCTTCCCAACAGTTGCGCAGCCTGAATGGCGAATGGCGATT |
| | | CCGTTGCAATGGCTGGCGGTAATATTGTTCTGGATATTACCAGCAAGGCC |
| | | GATAGTTTGAGTTCTTCTACTCAGGCAAGTGATGTTATTACTAATCAAAG |
| | | AAGTATTGCGACAACGGTTAATTTGCGTGATGGACAGACTCTTTTACTCG |
| | | GTGGCCTCACTGATTATAAAAACACTTCTCAGGATTCTGGCGTACCGTTC |
| | | CTGTCTAAAATCCCTTTAATCGGCCTCCTGTTTAGCTCCCGCTCTGATTCT |
| | | AACGAGGAAAGCACGTTATACGTGCTCGTCAAAGCAACCATAGTACGCG |
| | | CCCTGTAGCGGCGCATTAAGCGCGGCGGGTGTGGTGGTTACGCGCAGCGT |
| | | GACCGCTACACTTGCCAGCGCCCTAGCGCCCGCTCCTTTCGCTTTCTTCCC |
| | | TTCCTTTCTCGCCACGTTCGCCGGCTTTCCCCGTCAAGCTCTAAATCGGGG |
| | | GCTCCCTTTAGGGTTCCGATTTAGTGCTTTACGGCACCTCGACCCCAAAA |
| | | AACTTGATTAGGGTGATGGTTCACGTAGTGGGCCATCGCCCTGATAGACG |
| | | GTTTTTCGCCCTTTGACGTTGGAGTCCACGTTCTTTAATAGTGGACTCTTG |
| | | TTCCAAACTGGAACAACACTCAACCCTATCTCGGTCTATTCTTTTGATTTA |
| | | TAAGGGATTTTGCCGATTTCGGCCTATTGGTTAAAAAATGAGCTGATTTA |
| | | ACAAAAATTTAACGCGAATTTTAACAAAATATTAACGTTTACAATTTAAA |
| | | TATTTGCTTATACAATCTTCCTGTTTTTGGGGCTTTTCTGATTATCAACCGG |
| | | GGTACATATGATTGACATGCTAGTTTTACGATTACCGTTCATCGATTCTCT |
| | | TGTTTGCTCCAGACTCTCAGGCAATGACCTGATAGCCTTTGTAGAGACCT |
| | | CTCAAAAATAGCTACCCTCTCCGGCATGAATTTATCAGCTAGAACGGTTG |
| | | AATATCATATTGATGGTGATTTGACTGTCTCCGGCCTTTCTCACCCGTTTG |
| | | AATCTTTACCTACACATTACTCAGGCATTGCATTTAAAATATATGAGGGTT |
| | | CTAAAAATTTTTATCCTTGCGTTGAAATAAAGGCTTCTCCCGCAAAAGTA |
| | | TTACAGGGTCATAATGTTTTTGGTACAACCGATTTAGCTTTATGCTCTGAG |
| | | GCTTTATTGCTTAATTTTGCTAATTCTTTGCCTTGCCTGTATGATTTATTGG |
| | | ATGTTGGAATCGCCTGATGCGGTATTTTCTCCTTACGCATCTGTGCGGTAT |
| | | TTCACACCGCATATGGTGCACTCTCAGTACAATCTGCTCTGATGCCGCAT |
| | | AGTTAAGCCAGCCCCGACACCCGCCAACACCCGCTGACGCGCCCTGACG |
| | | GGCTTGTCTGCTCCCGGCATCCGCTTACAGACAAGCTGTGACCGTCTCCG |
| | | GGAGCTGCATGTGTCAGAGGTTTTCACCGTCATCACCGAAACGCGCGAGA |
| | | CGAAAGGGCCTCGTGATACGCCTATTTTTATAGGTTAATGTCATGATAAT |
| | | AATGGTTTCTTAGACGTCAGGTGGCACTTTTCGGGGAAATGTGCGCGGAA |
| | | CCCCTATTTGTTTATTTTTCTAAATACATTCAAATATGTATCCGCTCATGA |
| | | GACAATAACCCTGATAAATGCTTCAATAATATTGAAAAAGGAAGAGTAT |
| | | GAGTATTCAACATTTCCGTGTCGCCCTTATTCCCTTTTTTGCGGCATTTTGC |
| | | CTTCCTGTTTTTGCTCACCCAGAAACGCTGGTGAAAGTAAAAGATGCTGA |
| | | AGATCAGTTGGGTGCACGAGTGGGTTACATCGAACTGGATCTCAACAGCG |
| | | GTAAGATCCTTGAGAGTTTTCGCCCCGAAGAACGTTTTCCAATGATGAGC |
| | | ACTTTTAAAGTTCTGCTATGTGGCGCGGTATTATCCCGTATTGACGCCGGG |
| | | CAAGAGCAACTCGGTCGCCGCATACACTATTCTCAGAATGACTTGGTTGA |
| | | GTACTCACCAGTCACAGAAAAGCATCTTACGGATGGCATGACAGTAAGA |
| | | GAATTATGCAGTGCTGCCATAACCATGAGTGATAACACTGCGGCCAACTT |
| | | ACTTCTGACAACGATCGGAGGACCGAAGGAGCTAACCGCTTTTTTGCACA |
| | | ACATGGGGGATCATGTAACTCGCCTTGATCGTTGGGAACCGGAGCTGAAT |
| | | GAAGCCATACCAAACGACGAGCGTGACACCACGATGCCTGTAGCAATGG |
| | | CAACAACGTTGCGCAAACTATTAACTGGCGAACTACTTACTCTAGCTTCC |
| | | CGGCAACAATTAATAGACTGGATGGAGGCGGATAAAGTTGCAGGACCAC |
| | | TTCTGCGCTCGGCCCTTCCGGCTGGCTGGTTTATTGCTGATAAATCTGGAG |
| | | CCGGTGAGCGTGGGTCTCGCGGTATCATTGCAGCACTGGGGCCAGATGGT |
| | | AAGCCCTCCCGTATCGTAGTTATCTACACGACGGGGAGTCAGGCAACTAT |
| | | GGATGAACGAAATAGACAGATCGCTGAGATAGGTGCCTCACTGATTAAG |
| | | CATTGGTAACTGTCAGACCAAGTTTACTCATATATACTTTAGATTGATTTA |
| | | AAACTTCATTTTTAATTTAAAAGGATCTAGGTGAAGATCCTTTTTGATAAT |
| | | CTCATGACCAAAATCCCTTAACGTGAGTTTTCGTTCCACTGAGCGTCAGA |
| | | CCCC |
| 159 | #1392 pAAV- | GTAGAAAAGATCAAAGGATCTTCTTGAGATCCTTTTTTTCTGCGCGTAATC |
| | FOXP3_0.9[RV | TGCTGCTTGCAAACAAAAAAACCACCGCTACCAGCGGTGGTTTGTTTGCC |
| | S0.7UCOE- | GGATCAAGAGCTACCAACTCTTTTTCCGAAGGTAACTGGCTTCAGCAGAG |
| | MND- | CGCAGATACCAAATACTGTCCTTCTAGTGTAGCCGTAGTTAGGCCACCAC |
| | GFPki]_0.9 | TTCAAGAACTCTGTAGCACCGCCTACATACCTCGCTCTGCTAATCCTGTTA |
| | | CCAGTGGCTGCTGCCAGTGGCGATAAGTCGTGTCTTACCGGGTTGGACTC |
| | | AAGACGATAGTTACCGGATAAGGCGCAGCGGTCGGGCTGAACGGGGGGT |
| | | TCGTGCACACAGCCCAGCTTGGAGCGAACGACCTACACCGAACTGAGAT |
| | | ACCTACAGCGTGAGCTATGAGAAAGCGCCACGCTTCCCGAAGGGAGAAA |
| | | GGCGGACAGGTATCCGGTAAGCGGCAGGGTCGGAACAGGAGAGCGCACG |
| | | AGGGAGCTTCCAGGGGGAAACGCCTGGTATCTTTATAGTCCTGTCGGGTT |
| | | TCGCCACCTCTGACTTGAGCGTCGATTTTTGTGATGCTCGTCAGGGGGGC |
| | | GGAGCCTATGGAAAAACGCCAGCAACGCGGCCTTTTTACGGTTCCTGGCC |
| | | TTTTGCTGGCCTTTTGCTCACATGTTCTTTCCTGCGTTATCCCCTGATTCTG |
| | | TGGATAACCGTATTACCGCCTTTGAGTGAGCTGATACCGCTCGCCGCAGC |
| | | CGAACGACCGAGCGCAGCGAGTCAGTGAGCGAGGAAGCGGAAGAGCGC |
| | | CCAATACGCAAACCGCCTCTCCCCGCGCGTTGGCCGATTCATTAATGCAG |
| | | CTGCGCGCTCGCTCGCTCACTGAGGCCGCCCGGGCAAAGCCCGGGCGTCG |
| | | GGCGACCTTTGGTCGCCCGGCCTCAGTGAGCGAGCGAGCGCGCAGAGAG |
| | | GGAGTGGCCAACTCCATCACTAGGGGTTCCTTGTAGTTAATGATTAACCC |
| | | GCCATGCTACTTATCTACGTAGCGGCCGCGCTCAAGAGACCCCATCTCTC |
| | | CTCCTCTCTGTCACTTGCCATGCTGGATCCGTGCATGATCACACTCCTGGA |
| | | CTCGCCTCCTTGCCCTGAGATCCAGACCCCCGTATTCAGCTGCCCCCTCAG |
| | | CTCCTCCACTCACATATTTAATGCCAGACTCTTCATGTCTATCTACACCTG |
| | | CACTTTTGCACCCAATCCAACTCCCCGCCATGTCCCCCATCTCAGGTAATG |
| | | TCAGCTCGGTCCTTCCAGCTGCTCAAGCTAAAACCCATGTCACTTTGACTC |
| | | TCCCTCTTGCCCACTACATCCAAGCTGCTAGCACTGCTCCTGATCCAGCTT |
| | | CAGATTAAGTCTCAGAATCTACCCACTTCTCGCCTTCTCCACTGCCACCAG |
| | | CCCATTCTGTGCCAGCATCATCACTTGCCAGGACTGTTACAATAGCCTCCT |
| | | CACTAGCCCCACTCACAGCAGCCAGATGAATCTTTTGAGTCCATGCCTAG |
| | | TCACTGGGGCAAAATAGGACTCCGAGGAGAAAGTCCGAGACCAGCTCCG |
| | | GCAAGATGAGCAAACACAGCCTGTGCAGGGTGCAGGGAGGGCTAGAGGC |
| | | CTGAGGCTTGAAACAGCTCTCAAGTGGAGGGGGAAACAACCATTGCCCT |
| | | CATAGAGGACACATCCACACCAGGGCTGTGCTAGCGTGGGCAGGCAAGC |
| | | CAGGTGCTGGACCTCTGCACGTGGGGCATGTGTGGGTATGTACATGTACC |
| | | TGTGTTCTTGGTGTGTGTGTGTGTGTGTGTGTGTGTGTGTGTCTAGAGCTG |
| | | GGGTGCAACTATGGGGCCCCTCGGGACATGTCCCAGCCAATGCCTGCTTT |
| | | GACCAGAGGAGTGTCCACGTGGCTCAGGTGGTCGAGTATCTCATACCGCC |
| | | CTAGCACACGTGTGACTCCTTTCCCCTATTGTCTACCCGGGTGTGGCATCT |
| | | GAAGCACCACCAGCGAGCGAGAGCTAGAGAGAAGGAAAGCCACCGACTT |
| | | CACCGCCTCCGAGCTGCTCCGGGTCGCGGGTCTGCAGCGTCTCCGGCCCT |
| | | CCGCGCCTACAGCTCAAGCCACATCCGAAGGGGGAGGGAGCCGGGAGCT |
| | | GCGCGCGGGGCCGCCGGGGGGAGGGGTGGCACCGCCCACGCCGGGCGGC |
| | | CACGAAGGGCGGGGCAGCGGGCGCGCGCCCGGCGGGGGGAGGGGCCGC |
| | | GCGCCGCGCCCGCTGGGAATTGGGGCCCTAGGGGGAGGGCGGAGGCGCC |
| | | GACGACCGCGGCACTTACCGTTCGCGGCGTGGCGCCCGGTGGTCCCCAAG |
| | | GGGAGGGAAGGGGGAGGCGGGGCGAGGACAGTGACCGGAGTCTCCTCA |
| | | GCGGTGGCTTTTCTGCTTGGCAGCCTCAGCGGCTGGCGCCAAAACCGGAC |
| | | TCCGCCCACTTCCTCGCCCCTGCGGTGCGAGGGTGTGGAATCCTCCAGAC |
| | | GCTGGGGGAGGGGGAGTTGGGAGCTTAAAAACTAGTACCCCTTTGGGAC |
| | | CACTTTCAGCAGCGAACTCTCCTGTACACCAGGGGTCAGTTCCACAGACG |
| | | CGGGCCAGGGGTGGGTCATTGCGGCGTGAACAATAATTTGACTAGAAGTT |
| | | GATTCGGGTGTTTCCCGGGGAACAGAGAAACAGGAGAATATGGGCCAAA |
| | | CAGGATATCTGTGGTAAGCAGTTCCTGCCCCGGCTCAGGGCCAAGAACAG |
| | | TTGGAACAGCAGAATATGGGCCAAACAGGATATCTGTGGTAAGCAGTTC |
| | | CTGCCCCGGCTCAGGGCCAAGAACAGATGGTCCCCAGATGCGGTCCCGCC |
| | | CTCAGCAGTTTCTAGAGAACCATCAGATGTTTCCAGGGTGCCCCAAGGAC |
| | | CTGAAATGACCCTGTGCCTTATTTGAACTAACCAATCAGTTCGCTTCTCGC |
| | | TTCTGTTCGCGCGCTTCTGCTCCCCGAGCTCTATATAAGCAGAGCTCGTTT |
| | | AGTGAACCGTCAGATCGTCTACGCAGCCTGCCCTTGGACAAGGACCCGAT |
| | | GCCCAACCCCAGGCCTGTGAGCAAGGGCGAGGAGCTGTTCACCGGGGTG |
| | | GTGCCCATCCTGGTCGAGCTGGACGGCGACGTAAACGGCCACAAGTTCA |
| | | GCGTGTCCGGCGAGGGCGAGGGCGATGCCACCTACGGCAAGCTGACCCT |
| | | GAAGTTCATCTGCACCACCGGCAAGCTGCCCGTGCCCTGGCCCACCCTCG |
| | | TGACCACCCTGACCTACGGCGTGCAGTGCTTCAGCCGCTACCCCGACCAC |
| | | ATGAAGCAGCACGACTTCTTCAAGTCCGCCATGCCCGAAGGCTACGTCCA |
| | | GGAGCGCACCATCTTCTTCAAGGACGACGGCAACTACAAGACCCGCGCC |
| | | GAGGTGAAGTTCGAGGGCGACACCCTGGTGAACCGCATCGAGCTGAAGG |
| | | GCATCGACTTCAAGGAGGACGGCAACATCCTGGGGCACAAGCTGGAGTA |
| | | CAACTACAACAGCCACAACGTCTATATCATGGCCGACAAGCAGAAGAAC |
| | | GGCATCAAGGTGAACTTCAAGATCCGCCACAACATCGAGGACGGCAGCG |
| | | TGCAGCTCGCCGACCACTACCAGCAGAACACCCCCATCGGCGACGGCCCC |
| | | GTGCTGCTGCCCGACAACCACTACCTGAGCACCCAGTCCGCCCTGAGCAA |
| | | AGACCCCAACGAGAAGCGCGATCACATGGTCCTGCTGGAGTTCGTGACC |
| | | GCCGCCGGGATCACTCTCGGCATGGACGAGCTGTACAAGGGCAAGCCCT |
| | | CGGCCCCTTCCTTGGCCCTTGGCCCATCCCCAGGAGCCTCGCCCAGCTGG |
| | | AGGGCTGCCCCTAAAGCAAGCGACCTGCTGGGGGCCCGGGGCCCGGGTG |
| | | GCACGTTCCAGGGCCGAGATCTTCGAGGCGGGGCCCATGCCTCCTCTTCT |
| | | TCCTTGAACCCCATGCCACCATCGCAGCTGCAGGTGAGGCCCTGGGCCCA |
| | | GGATGGGGCAGGCAGGGTGGGGTACCTGGACCTACAGGTGCCGACCTTT |
| | | ACTGTGGCACTGGGCGGGAGGGGGGCTGGCTGGGGCACAGGAAGTGGTT |
| | | TCTGGGTCCCAGGCAAGTCTGTGACTTATGCAGATGTTGCAGGGCCAAGA |
| | | AAATCCCCACCTGCCAGGCCTCAGAGATTGGAGGCTCTCCCCGACCTCCC |
| | | AATCCCTGTCTCAGGAGAGGAGGAGGCCGTATTGTAGTCCCATGAGCATA |
| | | GCTATGTGTCCCCATCCCCATGTGACAAGAGAAGAGGACTGGGGCCAAG |
| | | TAGGTGAGGTGACAGGGCTGAGGCCAGCTCTGCAACTTATTAGCTGTTTG |
| | | ATCTTTAAAAAGTTACTCGATCTCCATGAGCCTCAGTTTCCATACGTGTAA |
| | | AAGGGGGATGATCATAGCATCTACCATGTGGGCTTGCAGTGCAGAGTATT |
| | | TGAATTAGACACAGAACAGTGAGGATCAGGATGGCCTCTCACCCACCTGC |
| | | CTTTCTGCCCAGCTGCCCACACTGCCCCTAGTCATGGTGGCACCCTCCGG |
| | | GGCACGGCTGGGCCCCTTGCCCCACTTACAGGCACTCCTCCAGGACAGGC |
| | | CACATTTCATGCACCAGGTATGGACGGTGAATGGGCAGGGAGGAGGGAG |
| | | CAGGTGGGAGAACTGTGGGGAGGGGCCCCGAGTCAGGCTGAACCACAGC |
| | | CCACATGGCGGCCGCTACGTAGATAAGTAGCATGGCGGGTTAATCATTAA |
| | | CTACAAGGAACCCCTAGTGATGGAGTTGGCCACTCCCTCTCTGCGCGCTC |
| | | GCTCGCTCACTGAGGCCGGGCGACCAAAGGTCGCCCGACGCCCGGGCTTT |
| | | GCCCGGGCGGCCTCAGTGAGCGAGCGAGCGCGCCAGCTGGCGTAATAGC |
| | | GAAGAGGCCCGCACCGATCGCCCTTCCCAACAGTTGCGCAGCCTGAATGG |
| | | CGAATGGCGATTCCGTTGCAATGGCTGGCGGTAATATTGTTCTGGATATT |
| | | ACCAGCAAGGCCGATAGTTTGAGTTCTTCTACTCAGGCAAGTGATGTTAT |
| | | TACTAATCAAAGAAGTATTGCGACAACGGTTAATTTGCGTGATGGACAGA |
| | | CTCTTTTACTCGGTGGCCTCACTGATTATAAAAACACTTCTCAGGATTCTG |
| | | GCGTACCGTTCCTGTCTAAAATCCCTTTAATCGGCCTCCTGTTTAGCTCCC |
| | | GCTCTGATTCTAACGAGGAAAGCACGTTATACGTGCTCGTCAAAGCAACC |
| | | ATAGTACGCGCCCTGTAGCGGCGCATTAAGCGCGGCGGGTGTGGTGGTTA |
| | | CGCGCAGCGTGACCGCTACACTTGCCAGCGCCCTAGCGCCCGCTCCTTTC |
| | | GCTTTCTTCCCTTCCTTTCTCGCCACGTTCGCCGGCTTTCCCCGTCAAGCTC |
| | | TAAATCGGGGGCTCCCTTTAGGGTTCCGATTTAGTGCTTTACGGCACCTCG |
| | | ACCCCAAAAAACTTGATTAGGGTGATGGTTCACGTAGTGGGCCATCGCCC |
| | | TGATAGACGGTTTTTCGCCCTTTGACGTTGGAGTCCACGTTCTTTAATAGT |
| | | GGACTCTTGTTCCAAACTGGAACAACACTCAACCCTATCTCGGTCTATTCT |
| | | TTTGATTTATAAGGGATTTTGCCGATTTCGGCCTATTGGTTAAAAAATGAG |
| | | CTGATTTAACAAAAATTTAACGCGAATTTTAACAAAATATTAACGTTTAC |
| | | AATTTAAATATTTGCTTATACAATCTTCCTGTTTTTGGGGCTTTTCTGATTA |
| | | TCAACCGGGGTACATATGATTGACATGCTAGTTTTACGATTACCGTTCATC |
| | | GATTCTCTTGTTTGCTCCAGACTCTCAGGCAATGACCTGATAGCCTTTGTA |
| | | GAGACCTCTCAAAAATAGCTACCCTCTCCGGCATGAATTTATCAGCTAGA |
| | | ACGGTTGAATATCATATTGATGGTGATTTGACTGTCTCCGGCCTTTCTCAC |
| | | CCGTTTGAATCTTTACCTACACATTACTCAGGCATTGCATTTAAAATATAT |
| | | GAGGGTTCTAAAAATTTTTATCCTTGCGTTGAAATAAAGGCTTCTCCCGC |
| | | AAAAGTATTACAGGGTCATAATGTTTTTGGTACAACCGATTTAGCTTTAT |
| | | GCTCTGAGGCTTTATTGCTTAATTTTGCTAATTCTTTGCCTTGCCTGTATGA |
| | | TTTATTGGATGTTGGAATCGCCTGATGCGGTATTTTCTCCTTACGCATCTG |
| | | TGCGGTATTTCACACCGCATATGGTGCACTCTCAGTACAATCTGCTCTGAT |
| | | GCCGCATAGTTAAGCCAGCCCCGACACCCGCCAACACCCGCTGACGCGCC |
| | | CTGACGGGCTTGTCTGCTCCCGGCATCCGCTTACAGACAAGCTGTGACCG |
| | | TCTCCGGGAGCTGCATGTGTCAGAGGTTTTCACCGTCATCACCGAAACGC |
| | | GCGAGACGAAAGGGCCTCGTGATACGCCTATTTTTATAGGTTAATGTCAT |
| | | GATAATAATGGTTTCTTAGACGTCAGGTGGCACTTTTCGGGGAAATGTGC |
| | | GCGGAACCCCTATTTGTTTATTTTTCTAAATACATTCAAATATGTATCCGC |
| | | TCATGAGACAATAACCCTGATAAATGCTTCAATAATATTGAAAAAGGAA |
| | | GAGTATGAGTATTCAACATTTCCGTGTCGCCCTTATTCCCTTTTTTGCGGC |
| | | ATTTTGCCTTCCTGTTTTTGCTCACCCAGAAACGCTGGTGAAAGTAAAAG |
| | | ATGCTGAAGATCAGTTGGGTGCACGAGTGGGTTACATCGAACTGGATCTC |
| | | AACAGCGGTAAGATCCTTGAGAGTTTTCGCCCCGAAGAACGTTTTCCAAT |
| | | GATGAGCACTTTTAAAGTTCTGCTATGTGGCGCGGTATTATCCCGTATTGA |
| | | CGCCGGGCAAGAGCAACTCGGTCGCCGCATACACTATTCTCAGAATGACT |
| | | TGGTTGAGTACTCACCAGTCACAGAAAAGCATCTTACGGATGGCATGACA |
| | | GTAAGAGAATTATGCAGTGCTGCCATAACCATGAGTGATAACACTGCGGC |
| | | CAACTTACTTCTGACAACGATCGGAGGACCGAAGGAGCTAACCGCTTTTT |
| | | TGCACAACATGGGGGATCATGTAACTCGCCTTGATCGTTGGGAACCGGAG |
| | | CTGAATGAAGCCATACCAAACGACGAGCGTGACACCACGATGCCTGTAG |
| | | CAATGGCAACAACGTTGCGCAAACTATTAACTGGCGAACTACTTACTCTA |
| | | GCTTCCCGGCAACAATTAATAGACTGGATGGAGGCGGATAAAGTTGCAG |
| | | GACCACTTCTGCGCTCGGCCCTTCCGGCTGGCTGGTTTATTGCTGATAAAT |
| | | CTGGAGCCGGTGAGCGTGGGTCTCGCGGTATCATTGCAGCACTGGGGCCA |
| | | GATGGTAAGCCCTCCCGTATCGTAGTTATCTACACGACGGGGAGTCAGGC |
| | | AACTATGGATGAACGAAATAGACAGATCGCTGAGATAGGTGCCTCACTG |
| | | ATTAAGCATTGGTAACTGTCAGACCAAGTTTACTCATATATACTTTAGATT |
| | | GATTTAAAACTTCATTTTTAATTTAAAAGGATCTAGGTGAAGATCCTTTTT |
| | | GATAATCTCATGACCAAAATCCCTTAACGTGAGTTTTCGTTCCACTGAGC |
| | | GTCAGACCCC |
| 160 | <#1331-pAAV- | CAGCTGCGCGCTCGCTCGCTCACTGAGGCCGCCCGGGCAAAGCCCGGGC |
| | mFOXP3-MND- | GTCGGGCGACCTTTGGTCGCCCGGCCTCAGTGAGCGAGCGAGCGCGCAG |
| | GFP-ki> for | AGAGGGAGTGGCCAACTCCATCACTAGGGGTTCCTTGTAGTTAATGATTA |
| | murine editing) | ACCCGCCATGCTACTTATCTACAGTATAGGATCCTGAAAAACGAAAGCCA |
| | | CACTTTTAAGGGACTGTAAGGTAGTGAGGCTCAGCACAGGGACCTGGGTC |
| | | ACCATGTAGAGCTTTGAAGAGGAAATCAGAAGACTGCAGTATGGCTAAG |
| | | GGAAGAAGTGGACTTCCAAGCTTGGCAGAGATTGGAGCTAGTTTGAGGA |
| | | GCGCCCAGGGACCCTCAATCAAGCAACCCTATCCCTCTTTTTTTCCTGGCA |
| | | CCTGCCACGCCAATTCCAAGACAGAAGAAAGCTTAGAGAAGACAGACCC |
| | | ATGCTGTGGCCCTGAGCTCTGCAGTACTGAATTCAGCTGCAAGTCTTCCCT |
| | | GCCTCTACTGCTTACCTTTGCATTTAGCCACATCTGACTATCACTGTATAC |
| | | TCTGCTCCTCCATCCTCTACCCTCCATCTCCAGTAATGCTCCTGTTGTAGC |
| | | TGCTTCTGCCAAAAACCTAGACATCATCTTGACCCTTTCTCTCATCTCCTC |
| | | CATCCAAGCTCCCGGCAACTTCTCCTGACTCTGCCTTCAGACGAGACTTG |
| | | GAAGACAGTCACATCTCAGCAGCTCCTCTGCCGTTATCCAGGTTGGTAGC |
| | | AGCAACACCACTCGCCTCACTATTGCAGTACACTTCCCACTAGCACAGTT |
| | | CCCTGGAGCCTTCCTGCTCACAGCATCCAACTGAATCTTGTGAGGCTATG |
| | | CCCAAGTCATTGGAATAAAAAGATGAGAAGAGAGTCCAAGACAAGCCCC |
| | | AGTAGAATCAGCAAAGACTATGTGGCCTGCACAGAGTGCAGGGGGTACT |
| | | GGAGGGTCCCACAAACCAACTCCCCATCACCCCACATTCACGACAGAGTG |
| | | GTATGGTGTATGTAAGCAAGTGAGGTGCTGGACATGTGCATGTGTAGAAT |
| | | ATATCCATCAATCTGTGTTCCTGCTGTCAGGGTAGCATATATGTATGTAAG |
| | | ACAGACCAGAGGTGTAGTTATGAGGCTATCTTGCACCACCCCTGGAATGC |
| | | ATGTGACTCCATTCCACTGTTACGCGTGAACAGAGAAACAGGAGAATATG |
| | | GGCCAAACAGGATATCTGTGGTAAGCAGTTCCTGCCCCGGCTCAGGGCCA |
| | | AGAACAGTTGGAACAGCAGAATATGGGCCAAACAGGATATCTGTGGTAA |
| | | GCAGTTCCTGCCCCGGCTCAGGGCCAAGAACAGATGGTCCCCAGATGCG |
| | | GTCCCGCCCTCAGCAGTTTCTAGAGAACCATCAGATGTTTCCAGGGTGCC |
| | | CCAAGGACCTGAAATGACCCTGTGCCTTATTTGAACTAACCAATCAGTTC |
| | | GCTTCTCGCTTCTGTTCGCGCGCTTCTGCTCCCCGAGCTCTATATAAGCAG |
| | | AGCTCGTTTAGTGAACCGTCAGATCGAATTCATCCCTGCAGCCTGCCTCT |
| | | GACAAGAACCCAATGCCCAACCCTAGGCCAGTGAGCAAGGGCGAGGAGC |
| | | TGTTCACCGGGGTGGTGCCCATCCTGGTCGAGCTGGACGGCGACGTAAAC |
| | | GGCCACAAGTTCAGCGTGTCCGGCGAGGGCGAGGGCGATGCCACCTACG |
| | | GCAAGCTGACCCTGAAGTTCATCTGCACCACCGGCAAGCTGCCCGTGCCC |
| | | TGGCCCACCCTCGTGACCACCCTGACCTACGGCGTGCAGTGCTTCAGCCG |
| | | CTACCCCGACCACATGAAGCAGCACGACTTCTTCAAGTCCGCCATGCCCG |
| | | AAGGCTACGTCCAGGAGCGCACCATCTTCTTCAAGGACGACGGCAACTAC |
| | | AAGACCCGCGCCGAGGTGAAGTTCGAGGGCGACACCCTGGTGAACCGCA |
| | | TCGAGCTGAAGGGCATCGACTTCAAGGAGGACGGCAACATCCTGGGGCA |
| | | CAAGCTGGAGTACAACTACAACAGCCACAACGTCTATATCATGGCCGAC |
| | | AAGCAGAAGAACGGCATCAAGGTGAACTTCAAGATCCGCCACAACATCG |
| | | AGGACGGCAGCGTGCAGCTCGCCGACCACTACCAGCAGAACACCCCCAT |
| | | CGGCGACGGCCCCGTGCTGCTGCCCGACAACCACTACCTGAGCACCCAGT |
| | | CCGCCCTGAGCAAAGACCCCAACGAGAAGCGCGATCACATGGTCCTGCT |
| | | GGAGTTCGTGACCGCCGCCGGGATCACTCTCGGCATGGACGAGCTGTACA |
| | | AGGCCAAGCCTATGGCTCCTTCGCTCGCGTTAGGGCCTAGCCCAGGAGTC |
| | | TTGCCTTCGTGGAAAACAGCACCCAAGGGCTCAGAACTTCTAGGGACCAG |
| | | GGGCTCTGGGGGACCCTTCCAAGGTCGGGACCTGCGAAGTGGGGCCCAC |
| | | ACCTCTTCTTCCTTGAACCCCCTGCCACCATCCCAGCTGCAGGTGAGGCCC |
| | | GGGGCCCAGAATGGGGTAAGCAGGGTGGGGTACTTGGGCCTATAGGTGT |
| | | CGACCTTTACTGTGGCATGTGGCGGGGGGGGGGGGGGGGGCTGGGGCAC |
| | | AGGAAGTGGTTTATGGGTCCCAGGCAAGTCTGACTTATGCAGATATTGCA |
| | | GGGCCAAGAAAATCCCCACTCTCCAGGCTTCAGAGATTCAAGGCTTTCCC |
| | | CACCCCTCCCAATCCTCATCCCGATAGGAGACCTTATGATTCCATGGACA |
| | | TAGCCATGTATCCTCATCCCACTGTGACGAGATGGCTGGGGCCCAAGAAG |
| | | GTAACAGTGTTGGGGCCAGCTCTACCCCTTGAAACTGTTGGACCTTGATA |
| | | CATTCACTCTCCACGAGCCTCAGATTCCACTGATGTGAACTGGATAGTTC |
| | | CATTGTTGCTACCGTGTGAGACTTTAGTAAAGAGCTAATGAATGAGACAC |
| | | AGAACTATTAAGATGAGGCTCATGGCATCTCATGGCATCTCCCTTCTCTCT |
| | | CCAGCTGCCTACAGTGCCCCTAGTCATGGTGGCACCGTCTGGGGCCCGAC |
| | | TAGGTCCCTCACCCCACCTACAGGCCCTTCTCCAGGACAGACCACACTTC |
| | | ATGCATCAGGTATGGAATCGGAGCAGGCTGGGAGGAGGGAACAAAGAG |
| | | GACAGCTGTGGAGCAGAGCCCCAAGCCCCGCTGAGCCATGGTCCATGTGT |
| | | TCCCCAGCTCTCCACTGTGGATGCCCATGCCCAGACCCCTGTGCTCCAAG |
| | | TGCGTCCACTGGACAACCCAGCCATGATCAGCCTCCCACCACCTTCTGCT |
| | | GCCACTGGGGTCTTCTCCCTCAAGGCCCGGCCTGGCCTGCCACCTGGTAA |
| | | CACCTTCACAGTATCTCCAAGTTCTCTAATCTTTGAGCATGTGCAATGTAA |
| | | ACTTTTCTGAATTATAGCCCTATGGAGGTATAGAAGGGTCTTAAGAGTCA |
| | | CGGAAACTCCAACCTCCAAAAAAAAAAATATCAGACTTAGAACCTTGAA |
| | | GACATAGAATGCAAAAAAAACCACAAATCGCTATTATCAGTCAAAATGC |
| | | CGTAGATAAGTAGCATGGCGGGTTAATCATTAACTACAAGGAACCCCTAG |
| | | TGATGGAGTTGGCCACTCCCTCTCTGCGCGCTCGCTCGCTCACTGAGGCC |
| | | GGGCGACCAAAGGTCGCCCGACGCCCGGGCTTTGCCCGGGCGGCCTCAG |
| | | TGAGCGAGCGAGCGCGCCAGCTGGCGTAATAGCGAAGAGGCCCGCACCG |
| | | ATCGCCCTTCCCAACAGTTGCGCAGCCTGAATGGCGAATGGCGATTCCGT |
| | | TGCAATGGCTGGCGGTAATATTGTTCTGGATATTACCAGCAAGGCCGATA |
| | | GTTTGAGTTCTTCTACTCAGGCAAGTGATGTTATTACTAATCAAAGAAGT |
| | | ATTGCGACAACGGTTAATTTGCGTGATGGACAGACTCTTTTACTCGGTGG |
| | | CCTCACTGATTATAAAAACACTTCTCAGGATTCTGGCGTACCGTTCCTGTC |
| | | TAAAATCCCTTTAATCGGCCTCCTGTTTAGCTCCCGCTCTGATTCTAACGA |
| | | GGAAAGCACGTTATACGTGCTCGTCAAAGCAACCATAGTACGCGCCCTGT |
| | | AGCGGCGCATTAAGCGCGGCGGGTGTGGTGGTTACGCGCAGCGTGACCG |
| | | CTACACTTGCCAGCGCCCTAGCGCCCGCTCCTTTCGCTTTCTTCCCTTCCTT |
| | | TCTCGCCACGTTCGCCGGCTTTCCCCGTCAAGCTCTAAATCGGGGGCTCCC |
| | | TTTAGGGTTCCGATTTAGTGCTTTACGGCACCTCGACCCCAAAAAACTTG |
| | | ATTAGGGTGATGGTTCACGTAGTGGGCCATCGCCCTGATAGACGGTTTTT |
| | | CGCCCTTTGACGTTGGAGTCCACGTTCTTTAATAGTGGACTCTTGTTCCAA |
| | | ACTGGAACAACACTCAACCCTATCTCGGTCTATTCTTTTGATTTATAAGGG |
| | | ATTTTGCCGATTTCGGCCTATTGGTTAAAAAATGAGCTGATTTAACAAAA |
| | | ATTTAACGCGAATTTTAACAAAATATTAACGTTTACAATTTAAATATTTGC |
| | | TTATACAATCTTCCTGTTTTTGGGGCTTTTCTGATTATCAACCGGGGTACA |
| | | TATGATTGACATGCTAGTTTTACGATTACCGTTCATCGATTCTCTTGTTTG |
| | | CTCCAGACTCTCAGGCAATGACCTGATAGCCTTTGTAGAGACCTCTCAAA |
| | | AATAGCTACCCTCTCCGGCATGAATTTATCAGCTAGAACGGTTGAATATC |
| | | ATATTGATGGTGATTTGACTGTCTCCGGCCTTTCTCACCCGTTTGAATCTT |
| | | TACCTACACATTACTCAGGCATTGCATTTAAAATATATGAGGGTTCTAAA |
| | | AATTTTTATCCTTGCGTTGAAATAAAGGCTTCTCCCGCAAAAGTATTACA |
| | | GGGTCATAATGTTTTTGGTACAACCGATTTAGCTTTATGCTCTGAGGCTTT |
| | | ATTGCTTAATTTTGCTAATTCTTTGCCTTGCCTGTATGATTTATTGGATGTT |
| | | GGAATCGCCTGATGCGGTATTTTCTCCTTACGCATCTGTGCGGTATTTCAC |
| | | ACCGCATATGGTGCACTCTCAGTACAATCTGCTCTGATGCCGCATAGTTA |
| | | AGCCAGCCCCGACACCCGCCAACACCCGCTGACGCGCCCTGACGGGCTTG |
| | | TCTGCTCCCGGCATCCGCTTACAGACAAGCTGTGACCGTCTCCGGGAGCT |
| | | GCATGTGTCAGAGGTTTTCACCGTCATCACCGAAACGCGCGAGACGAAA |
| | | GGGCCTCGTGATACGCCTATTTTTATAGGTTAATGTCATGATAATAATGGT |
| | | TTCTTAGACGTCAGGTGGCACTTTTCGGGGAAATGTGCGCGGAACCCCTA |
| | | TTTGTTTATTTTTCTAAATACATTCAAATATGTATCCGCTCATGAGACAAT |
| | | AACCCTGATAAATGCTTCAATAATATTGAAAAAGGAAGAGTATGAGTATT |
| | | CAACATTTCCGTGTCGCCCTTATTCCCTTTTTTGCGGCATTTTGCCTTCCTG |
| | | TTTTTGCTCACCCAGAAACGCTGGTGAAAGTAAAAGATGCTGAAGATCAG |
| | | TTGGGTGCACGAGTGGGTTACATCGAACTGGATCTCAACAGCGGTAAGAT |
| | | CCTTGAGAGTTTTCGCCCCGAAGAACGTTTTCCAATGATGAGCACTTTTA |
| | | AAGTTCTGCTATGTGGCGCGGTATTATCCCGTATTGACGCCGGGCAAGAG |
| | | CAACTCGGTCGCCGCATACACTATTCTCAGAATGACTTGGTTGAGTACTC |
| | | ACCAGTCACAGAAAAGCATCTTACGGATGGCATGACAGTAAGAGAATTA |
| | | TGCAGTGCTGCCATAACCATGAGTGATAACACTGCGGCCAACTTACTTCT |
| | | GACAACGATCGGAGGACCGAAGGAGCTAACCGCTTTTTTGCACAACATG |
| | | GGGGATCATGTAACTCGCCTTGATCGTTGGGAACCGGAGCTGAATGAAGC |
| | | CATACCAAACGACGAGCGTGACACCACGATGCCTGTAGCAATGGCAACA |
| | | ACGTTGCGCAAACTATTAACTGGCGAACTACTTACTCTAGCTTCCCGGCA |
| | | ACAATTAATAGACTGGATGGAGGCGGATAAAGTTGCAGGACCACTTCTG |
| | | CGCTCGGCCCTTCCGGCTGGCTGGTTTATTGCTGATAAATCTGGAGCCGG |
| | | TGAGCGTGGGTCTCGCGGTATCATTGCAGCACTGGGGCCAGATGGTAAGC |
| | | CCTCCCGTATCGTAGTTATCTACACGACGGGGAGTCAGGCAACTATGGAT |
| | | GAACGAAATAGACAGATCGCTGAGATAGGTGCCTCACTGATTAAGCATT |
| | | GGTAACTGTCAGACCAAGTTTACTCATATATACTTTAGATTGATTTAAAA |
| | | CTTCATTTTTAATTTAAAAGGATCTAGGTGAAGATCCTTTTTGATAATCTC |
| | | ATGACCAAAATCCCTTAACGTGAGTTTTCGTTCCACTGAGCGTCAGACCC |
| | | CGTAGAAAAGATCAAAGGATCTTCTTGAGATCCTTTTTTTCTGCGCGTAAT |
| | | CTGCTGCTTGCAAACAAAAAAACCACCGCTACCAGCGGTGGTTTGTTTGC |
| | | CGGATCAAGAGCTACCAACTCTTTTTCCGAAGGTAACTGGCTTCAGCAGA |
| | | GCGCAGATACCAAATACTGTCCTTCTAGTGTAGCCGTAGTTAGGCCACCA |
| | | CTTCAAGAACTCTGTAGCACCGCCTACATACCTCGCTCTGCTAATCCTGTT |
| | | ACCAGTGGCTGCTGCCAGTGGCGATAAGTCGTGTCTTACCGGGTTGGACT |
| | | CAAGACGATAGTTACCGGATAAGGCGCAGCGGTCGGGCTGAACGGGGGG |
| | | TTCGTGCACACAGCCCAGCTTGGAGCGAACGACCTACACCGAACTGAGAT |
| | | ACCTACAGCGTGAGCTATGAGAAAGCGCCACGCTTCCCGAAGGGAGAAA |
| | | GGCGGACAGGTATCCGGTAAGCGGCAGGGTCGGAACAGGAGAGCGCACG |
| | | AGGGAGCTTCCAGGGGGAAACGCCTGGTATCTTTATAGTCCTGTCGGGTT |
| | | TCGCCACCTCTGACTTGAGCGTCGATTTTTGTGATGCTCGTCAGGGGGGC |
| | | GGAGCCTATGGAAAAACGCCAGCAACGCGGCCTTTTTACGGTTCCTGGCC |
| | | TTTTGCTGGCCTTTTGCTCACATGTTCTTTCCTGCGTTATCCCCTGATTCTG |
| | | TGGATAACCGTATTACCGCCTTTGAGTGAGCTGATACCGCTCGCCGCAGC |
| | | CGAACGACCGAGCGCAGCGAGTCAGTGAGCGAGGAAGCGGAAGAGCGC |
| | | CCAATACGCAAACCGCCTCTCCCCGCGCGTTGGCCGATTCATTAATG |
| 161 | <#3209 | CAGCTGCGCGCTCGCTCGCTCACTGAGGCCGCCCGGGCAAAGCCCGGGC |
| | pAAV_mFOXP | GTCGGGCGACCTTTGGTCGCCCGGCCTCAGTGAGCGAGCGAGCGCGCAG |
| | 3.06_PGK.GFPk | AGAGGGAGTGGCCAACTCCATCACTAGGGGTTCCTTGTAGTTAATGATTA |
| | i_06 for | ACCCGCCATGCTACTTATCTACGTAGCGGCCGCCTCCCGGCAACTTCTCCT |
| | mT23_from | GACTCTGCCTTCAGACGAGACTTGGAAGACAGTCACATCTCAGCAGCTCC |
| | 3171> (for | TCTGCCGTTATCCAGGTTGGTAGCAGCAACACCACTCGCCTCACTATTGC |
| | murine editing) | AGTACACTTCCCACTAGCACAGTTCCCTGGAGCCTTCCTGCTCACAGCAT |
| | | CCAACTGAATCTTGTGAGGCTATGCCCAAGTCATTGGAATAAAAAGATGA |
| | | GAAGAGAGTCCAAGACAAGCCCCAGTAGAATCAGCAAAGACTATGTGGC |
| | | CTGCACAGAGTGCAGGGGGTACTGGAGGGTCCCACAAACCAACTCCCCA |
| | | TCACCCCACATTCACGACAGAGTGGTATGGTGTATGTAAGCAAGTGAGGT |
| | | GCTGGACATGTGCATGTGTAGAATATATCCATCAATCTGTGTTCCTGCTGT |
| | | CAGGGTAGCATATATGTATGTAAGACAGACCAGAGGTGTAGTTATGAGG |
| | | CTATCTTGCACCACCCCTGGAATGCATGTGACTCCATTCCACTGTTATCCC |
| | | TGCAGCCTGCCTCTGACAAGAACCCAATGCCCAACCCTAGGCCAGCCAAG |
| | | CCTATGGCTCCTTCCTTGGCCCTTGGCCCATCCCCAGACGCGTCCACGGG |
| | | GTTGGGGTTGCGCCTTTTCCAAGGCAGCCCTGGGTTTGCGCAGGGACGCG |
| | | GCTGCTCTGGGCGTGGTTCCGGGAAACGCAGCGGCGCCGACCCTGGGTCT |
| | | CGCACATTCTTCACGTCCGTTCGCAGCGTCACCCGGATCTTCGCCGCTACC |
| | | CTTGTGGGCCCCCCGGCGACGCTTCCTGCTCCGCCCCTAAGTCGGGAAGG |
| | | TTCCTTGCGGTTCGCGGCGTGCCGGACGTGACAAACGGAAGCCGCACGTC |
| | | TCACTAGTACCCTCGCAGACGGACAGCGCCAGGGAGCAATGGCAGCGCG |
| | | CCGACCGCGATGGGCTGTGGCCAATAGCGGCTGCTCAGCGGGGCGCGCC |
| | | GAGAGCAGCGGCCGGGAAGGGGCGGTGCGGGAGGCGGGGTGTGGGGCG |
| | | GTAGTGTGGGCCCTGTTCCTGCCCGCGCGGTGAATTCATCCCTGCAGCCT |
| | | GCCTCTGACAAGAACCCAATGCCCAACCCTAGGCCAGTGAGCAAGGGCG |
| | | AGGAGCTGTTCACCGGGGTGGTGCCCATCCTGGTCGAGCTGGACGGCGAC |
| | | GTAAACGGCCACAAGTTCAGCGTGTCCGGCGAGGGCGAGGGCGATGCCA |
| | | CCTACGGCAAGCTGACCCTGAAGTTCATCTGCACCACCGGCAAGCTGCCC |
| | | GTGCCCTGGCCCACCCTCGTGACCACCCTGACCTACGGCGTGCAGTGCTT |
| | | CAGCCGCTACCCCGACCACATGAAGCAGCACGACTTCTTCAAGTCCGCCA |
| | | TGCCCGAAGGCTACGTCCAGGAGCGCACCATCTTCTTCAAGGACGACGGC |
| | | AACTACAAGACCCGCGCCGAGGTGAAGTTCGAGGGCGACACCCTGGTGA |
| | | ACCGCATCGAGCTGAAGGGCATCGACTTCAAGGAGGACGGCAACATCCT |
| | | GGGGCACAAGCTGGAGTACAACTACAACAGCCACAACGTCTATATCATG |
| | | GCCGACAAGCAGAAGAACGGCATCAAGGTGAACTTCAAGATCCGCCACA |
| | | ACATCGAGGACGGCAGCGTGCAGCTCGCCGACCACTACCAGCAGAACAC |
| | | CCCCATCGGCGACGGCCCCGTGCTGCTGCCCGACAACCACTACCTGAGCA |
| | | CCCAGTCCGCCCTGAGCAAAGACCCCAACGAGAAGCGCGATCACATGGT |
| | | CCTGCTGGAGTTCGTGACCGCCGCCGGGATCACTCTCGGCATGGACGAGC |
| | | TGTACAAGGCCAAGCCTATGGCTCCTTCGCTCGCGTTAGGGCCTAGCCCA |
| | | GGAGTCTTGCCTTCGTGGAAAACAGCACCCAAGGGCTCAGAACTTCTAGG |
| | | GACCAGGGGCTCTGGGGGACCCTTCCAAGGTCGGGACCTGCGAAGTGGG |
| | | GCCCACACCTCTTCTTCCTTGAACCCCCTGCCACCATCCCAGCTGCAGGTG |
| | | AGGCCCGGGGCCCAGAATGGGGTAAGCAGGGTGGGGTACTTGGGCCTAT |
| | | AGGTGTCGACCTTTACTGTGGCATGTGGCGGGGGGGGGGGGGGGGGCTG |
| | | GGGCACAGGAAGTGGTTTATGGGTCCCAGGCAAGTCTGACTTATGCAGAT |
| | | ATTGCAGGGCCAAGAAAATCCCCACTCTCCAGGCTTCAGAGATTCAAGGC |
| | | TTTCCCCACCCCTCCCAATCCTCATCCCGATAGGAGACCTTATGATTCCAT |
| | | GGACATAGCCATGTATCCTCATCCCACTGTGACGAGATGGCTGGGGCCCA |
| | | AGAAGGTAACAGTGTTGGGGCCAGCTCTACCCCTTGAAACTGTTGGACCT |
| | | TGATACATTCACTCTCCACGAGCCTCAGATTCCACTGATGTGAACTGGAT |
| | | AGTTCCATTGTTGCTACCGTGTGAGACTTTAGTAAAGAGCTAATGAATGA |
| | | GACACAGGCTAGCTACGTAGATAAGTAGCATGGCGGGTTAATCATTAACT |
| | | ACAAGGAACCCCTAGTGATGGAGTTGGCCACTCCCTCTCTGCGCGCTCGC |
| | | TCGCTCACTGAGGCCGGGCGACCAAAGGTCGCCCGACGCCCGGGCTTTGC |
| | | CCGGGCGGCCTCAGTGAGCGAGCGAGCGCGCCAGCTGGCGTAATAGCGA |
| | | AGAGGCCCGCACCGATCGCCCTTCCCAACAGTTGCGCAGCCTGAATGGCG |
| | | AATGGCGATTCCGTTGCAATGGCTGGCGGTAATATTGTTCTGGATATTAC |
| | | CAGCAAGGCCGATAGTTTGAGTTCTTCTACTCAGGCAAGTGATGTTATTA |
| | | CTAATCAAAGAAGTATTGCGACAACGGTTAATTTGCGTGATGGACAGACT |
| | | CTTTTACTCGGTGGCCTCACTGATTATAAAAACACTTCTCAGGATTCTGGC |
| | | GTACCGTTCCTGTCTAAAATCCCTTTAATCGGCCTCCTGTTTAGCTCCCGC |
| | | TCTGATTCTAACGAGGAAAGCACGTTATACGTGCTCGTCAAAGCAACCAT |
| | | AGTACGCGCCCTGTAGCGGCGCATTAAGCGCGGCGGGTGTGGTGGTTACG |
| | | CGCAGCGTGACCGCTACACTTGCCAGCGCCCTAGCGCCCGCTCCTTTCGC |
| | | TTTCTTCCCTTCCTTTCTCGCCACGTTCGCCGGCTTTCCCCGTCAAGCTCTA |
| | | AATCGGGGGCTCCCTTTAGGGTTCCGATTTAGTGCTTTACGGCACCTCGA |
| | | CCCCAAAAAACTTGATTAGGGTGATGGTTCACGTAGTGGGCCATCGCCCT |
| | | GATAGACGGTTTTTCGCCCTTTGACGTTGGAGTCCACGTTCTTTAATAGTG |
| | | GACTCTTGTTCCAAACTGGAACAACACTCAACCCTATCTCGGTCTATTCTT |
| | | TTGATTTATAAGGGATTTTGCCGATTTCGGCCTATTGGTTAAAAAATGAG |
| | | CTGATTTAACAAAAATTTAACGCGAATTTTAACAAAATATTAACGTTTAC |
| | | AATTTAAATATTTGCTTATACAATCTTCCTGTTTTTGGGGCTTTTCTGATTA |
| | | TCAACCGGGGTACATATGATTGACATGCTAGTTTTACGATTACCGTTCATC |
| | | GATTCTCTTGTTTGCTCCAGACTCTCAGGCAATGACCTGATAGCCTTTGTA |
| | | GAGACCTCTCAAAAATAGCTACCCTCTCCGGCATGAATTTATCAGCTAGA |
| | | ACGGTTGAATATCATATTGATGGTGATTTGACTGTCTCCGGCCTTTCTCAC |
| | | CCGTTTGAATCTTTACCTACACATTACTCAGGCATTGCATTTAAAATATAT |
| | | GAGGGTTCTAAAAATTTTTATCCTTGCGTTGAAATAAAGGCTTCTCCCGC |
| | | AAAAGTATTACAGGGTCATAATGTTTTTGGTACAACCGATTTAGCTTTAT |
| | | GCTCTGAGGCTTTATTGCTTAATTTTGCTAATTCTTTGCCTTGCCTGTATGA |
| | | TTTATTGGATGTTGGAATCGCCTGATGCGGTATTTTCTCCTTACGCATCTG |
| | | TGCGGTATTTCACACCGCATATGGTGCACTCTCAGTACAATCTGCTCTGAT |
| | | GCCGCATAGTTAAGCCAGCCCCGACACCCGCCAACACCCGCTGACGCGCC |
| | | CTGACGGGCTTGTCTGCTCCCGGCATCCGCTTACAGACAAGCTGTGACCG |
| | | TCTCCGGGAGCTGCATGTGTCAGAGGTTTTCACCGTCATCACCGAAACGC |
| | | GCGAGACGAAAGGGCCTCGTGATACGCCTATTTTTATAGGTTAATGTCAT |
| | | GATAATAATGGTTTCTTAGACGTCAGGTGGCACTTTTCGGGGAAATGTGC |
| | | GCGGAACCCCTATTTGTTTATTTTTCTAAATACATTCAAATATGTATCCGC |
| | | TCATGAGACAATAACCCTGATAAATGCTTCAATAATATTGAAAAAGGAA |
| | | GAGTATGAGTATTCAACATTTCCGTGTCGCCCTTATTCCCTTTTTTGCGGC |
| | | ATTTTGCCTTCCTGTTTTTGCTCACCCAGAAACGCTGGTGAAAGTAAAAG |
| | | ATGCTGAAGATCAGTTGGGTGCACGAGTGGGTTACATCGAACTGGATCTC |
| | | AACAGCGGTAAGATCCTTGAGAGTTTTCGCCCCGAAGAACGTTTTCCAAT |
| | | GATGAGCACTTTTAAAGTTCTGCTATGTGGCGCGGTATTATCCCGTATTGA |
| | | CGCCGGGCAAGAGCAACTCGGTCGCCGCATACACTATTCTCAGAATGACT |
| | | TGGTTGAGTACTCACCAGTCACAGAAAAGCATCTTACGGATGGCATGACA |
| | | GTAAGAGAATTATGCAGTGCTGCCATAACCATGAGTGATAACACTGCGGC |
| | | CAACTTACTTCTGACAACGATCGGAGGACCGAAGGAGCTAACCGCTTTTT |
| | | TGCACAACATGGGGGATCATGTAACTCGCCTTGATCGTTGGGAACCGGAG |
| | | CTGAATGAAGCCATACCAAACGACGAGCGTGACACCACGATGCCTGTAG |
| | | CAATGGCAACAACGTTGCGCAAACTATTAACTGGCGAACTACTTACTCTA |
| | | GCTTCCCGGCAACAATTAATAGACTGGATGGAGGCGGATAAAGTTGCAG |
| | | GACCACTTCTGCGCTCGGCCCTTCCGGCTGGCTGGTTTATTGCTGATAAAT |
| | | CTGGAGCCGGTGAGCGTGGGTCTCGCGGTATCATTGCAGCACTGGGGCCA |
| | | GATGGTAAGCCCTCCCGTATCGTAGTTATCTACACGACGGGGAGTCAGGC |
| | | AACTATGGATGAACGAAATAGACAGATCGCTGAGATAGGTGCCTCACTG |
| | | ATTAAGCATTGGTAACTGTCAGACCAAGTTTACTCATATATACTTTAGATT |
| | | GATTTAAAACTTCATTTTTAATTTAAAAGGATCTAGGTGAAGATCCTTTTT |
| | | GATAATCTCATGACCAAAATCCCTTAACGTGAGTTTTCGTTCCACTGAGC |
| | | GTCAGACCCCGTAGAAAAGATCAAAGGATCTTCTTGAGATCCTTTTTTTCT |
| | | GCGCGTAATCTGCTGCTTGCAAACAAAAAAACCACCGCTACCAGCGGTG |
| | | GTTTGTTTGCCGGATCAAGAGCTACCAACTCTTTTTCCGAAGGTAACTGG |
| | | CTTCAGCAGAGCGCAGATACCAAATACTGTCCTTCTAGTGTAGCCGTAGT |
| | | TAGGCCACCACTTCAAGAACTCTGTAGCACCGCCTACATACCTCGCTCTG |
| | | CTAATCCTGTTACCAGTGGCTGCTGCCAGTGGCGATAAGTCGTGTCTTAC |
| | | CGGGTTGGACTCAAGACGATAGTTACCGGATAAGGCGCAGCGGTCGGGC |
| | | TGAACGGGGGGTTCGTGCACACAGCCCAGCTTGGAGCGAACGACCTACA |
| | | CCGAACTGAGATACCTACAGCGTGAGCTATGAGAAAGCGCCACGCTTCCC |
| | | GAAGGGAGAAAGGCGGACAGGTATCCGGTAAGCGGCAGGGTCGGAACA |
| | | GGAGAGCGCACGAGGGAGCTTCCAGGGGGAAACGCCTGGTATCTTTATA |
| | | GTCCTGTCGGGTTTCGCCACCTCTGACTTGAGCGTCGATTTTTGTGATGCT |
| | | CGTCAGGGGGGCGGAGCCTATGGAAAAACGCCAGCAACGCGGCCTTTTT |
| | | ACGGTTCCTGGCCTTTTGCTGGCCTTTTGCTCACATGTTCTTTCCTGCGTTA |
| | | TCCCCTGATTCTGTGGATAACCGTATTACCGCCTTTGAGTGAGCTGATACC |
| | | GCTCGCCGCAGCCGAACGACCGAGCGCAGCGAGTCAGTGAGCGAGGAAG |
| | | CGGAAGAGCGCCCAATACGCAAACCGCCTCTCCCCGCGCGTTGGCCGATT |
| | | CATTAATG |
| 162 | <#3213 | CAGCTGCGCGCTCGCTCGCTCACTGAGGCCGCCCGGGCAAAGCCCGGGC |
| | pAAV_mFOXP | GTCGGGCGACCTTTGGTCGCCCGGCCTCAGTGAGCGAGCGAGCGCGCAG |
| | 3.06_07UCOErv | AGAGGGAGTGGCCAACTCCATCACTAGGGGTTCCTTGTAGTTAATGATTA |
| | s.MND.GFPki_0 | ACCCGCCATGCTACTTATCTACGTAGCGGCCGCCTCCCGGCAACTTCTCCT |
| | 6 from 3171> | GACTCTGCCTTCAGACGAGACTTGGAAGACAGTCACATCTCAGCAGCTCC |
| | | TCTGCCGTTATCCAGGTTGGTAGCAGCAACACCACTCGCCTCACTATTGC |
| | | AGTACACTTCCCACTAGCACAGTTCCCTGGAGCCTTCCTGCTCACAGCAT |
| | | CCAACTGAATCTTGTGAGGCTATGCCCAAGTCATTGGAATAAAAAGATGA |
| | | GAAGAGAGTCCAAGACAAGCCCCAGTAGAATCAGCAAAGACTATGTGGC |
| | | CTGCACAGAGTGCAGGGGGTACTGGAGGGTCCCACAAACCAACTCCCCA |
| | | TCACCCCACATTCACGACAGAGTGGTATGGTGTATGTAAGCAAGTGAGGT |
| | | GCTGGACATGTGCATGTGTAGAATATATCCATCAATCTGTGTTCCTGCTGT |
| | | CAGGGTAGCATATATGTATGTAAGACAGACCAGAGGTGTAGTTATGAGG |
| | | CTATCTTGCACCACCCCTGGAATGCATGTGACTCCATTCCACTGTTATCCC |
| | | TGCAGCCTGCCTCTGACAAGAACCCAATGCCCAACCCTAGGCCAGCCAAG |
| | | CCTATGGCTCCTTCCTTGGCCCTTGGCCCATCCCCAGACGCGTATCGATCA |
| | | CGAGACTAGCCTCGAAATTCGAGCTAGTCCCGGCCGCGTGTGGCATCTGA |
| | | AGCACCACCAGCGAGCGAGAGCTAGAGAGAAGGAAAGCCACCGACTTCA |
| | | CCGCCTCCGAGCTGCTCCGGGTCGCGGGTCTGCAGCGTCTCCGGCCCTCC |
| | | GCGCCTACAGCTCAAGCCACATCCGAAGGGGGAGGGAGCCGGGAGCTGC |
| | | GCGCGGGGCCGCTGGGGGGAGGGGTGGCACCGCCCACGCCGGGCGGCCA |
| | | CGAAGGGCGGGGCAGCGGGCGCGCGCCCGGCGGGGGGAGGGGCCGCGC |
| | | GCCGCGCCCGCTGGGAATTGGGGCCCTAGGGGGAGGGCGGAGGCGCCGA |
| | | CGACCGCGGCACTTACCGTTCGCGGCGTGGCGCCCGGTGGTCCCCAAGGG |
| | | GAGGGAAGGGGGAGGCGGGGCGAGGACAGTGACCGGAGTCTCCTCAGC |
| | | GGTGGCTTTTCTGCTTGGCAGCCTCAGCGGCTGGCGCCAAAACCGGACTC |
| | | CGCCCACTTCCTCGCCCCTGCGGTGCGAGGGTGTGGAATCCTCCAGACGC |
| | | TGGGGGAGGGGGAGTTGGGAGCTTAAAAACTAGTACCCCTTTGGGACCA |
| | | CTTTCAGCAGCGAACTCTCCTGTACACCAGGGGTCAGTTCCACAGACGCG |
| | | GGCCAGGGGTGGGTCATTGCGGCGTGAACAATAATTTGACTAGAAGTTG |
| | | ATTCGGGTGTTTGCGGCCGGGGCTAGCTACGACGCGTGAACAGAGAAAC |
| | | AGGAGAATATGGGCCAAACAGGATATCTGTGGTAAGCAGTTCCTGCCCC |
| | | GGCTCAGGGCCAAGAACAGTTGGAACAGCAGAATATGGGCCAAACAGGA |
| | | TATCTGTGGTAAGCAGTTCCTGCCCCGGCTCAGGGCCAAGAACAGATGGT |
| | | CCCCAGATGCGGTCCCGCCCTCAGCAGTTTCTAGAGAACCATCAGATGTT |
| | | TCCAGGGTGCCCCAAGGACCTGAAATGACCCTGTGCCTTATTTGAACTAA |
| | | CCAATCAGTTCGCTTCTCGCTTCTGTTCGCGCGCTTCTGCTCCCCGAGCTC |
| | | TATATAAGCAGAGCTCGTTTAGTGAACCGTCAGATCGAATTCATCCCTGC |
| | | AGCCTGCCTCTGACAAGAACCCAATGCCCAACCCTAGGCCAGTGAGCAA |
| | | GGGCGAGGAGCTGTTCACCGGGGTGGTGCCCATCCTGGTCGAGCTGGAC |
| | | GGCGACGTAAACGGCCACAAGTTCAGCGTGTCCGGCGAGGGCGAGGGCG |
| | | ATGCCACCTACGGCAAGCTGACCCTGAAGTTCATCTGCACCACCGGCAAG |
| | | CTGCCCGTGCCCTGGCCCACCCTCGTGACCACCCTGACCTACGGCGTGCA |
| | | GTGCTTCAGCCGCTACCCCGACCACATGAAGCAGCACGACTTCTTCAAGT |
| | | CCGCCATGCCCGAAGGCTACGTCCAGGAGCGCACCATCTTCTTCAAGGAC |
| | | GACGGCAACTACAAGACCCGCGCCGAGGTGAAGTTCGAGGGCGACACCC |
| | | TGGTGAACCGCATCGAGCTGAAGGGCATCGACTTCAAGGAGGACGGCAA |
| | | CATCCTGGGGCACAAGCTGGAGTACAACTACAACAGCCACAACGTCTAT |
| | | ATCATGGCCGACAAGCAGAAGAACGGCATCAAGGTGAACTTCAAGATCC |
| | | GCCACAACATCGAGGACGGCAGCGTGCAGCTCGCCGACCACTACCAGCA |
| | | GAACACCCCCATCGGCGACGGCCCCGTGCTGCTGCCCGACAACCACTACC |
| | | TGAGCACCCAGTCCGCCCTGAGCAAAGACCCCAACGAGAAGCGCGATCA |
| | | CATGGTCCTGCTGGAGTTCGTGACCGCCGCCGGGATCACTCTCGGCATGG |
| | | ACGAGCTGTACAAGGCCAAGCCTATGGCTCCTTCGCTCGCGTTAGGGCCT |
| | | AGCCCAGGAGTCTTGCCTTCGTGGAAAACAGCACCCAAGGGCTCAGAAC |
| | | TTCTAGGGACCAGGGGCTCTGGGGGACCCTTCCAAGGTCGGGACCTGCGA |
| | | AGTGGGGCCCACACCTCTTCTTCCTTGAACCCCCTGCCACCATCCCAGCTG |
| | | CAGGTGAGGCCCGGGGCCCAGAATGGGGTAAGCAGGGTGGGGTACTTGG |
| | | GCCTATAGGTGTCGACCTTTACTGTGGCATGTGGCGGGGGGGGGGGGGG |
| | | GGGCTGGGGCACAGGAAGTGGTTTATGGGTCCCAGGCAAGTCTGACTTAT |
| | | GCAGATATTGCAGGGCCAAGAAAATCCCCACTCTCCAGGCTTCAGAGATT |
| | | CAAGGCTTTCCCCACCCCTCCCAATCCTCATCCCGATAGGAGACCTTATG |
| | | ATTCCATGGACATAGCCATGTATCCTCATCCCACTGTGACGAGATGGCTG |
| | | GGGCCCAAGAAGGTAACAGTGTTGGGGCCAGCTCTACCCCTTGAAACTGT |
| | | TGGACCTTGATACATTCACTCTCCACGAGCCTCAGATTCCACTGATGTGA |
| | | ACTGGATAGTTCCATTGTTGCTACCGTGTGAGACTTTAGTAAAGAGCTAA |
| | | TGAATGAGACACAGGCTAGCTACGTAGATAAGTAGCATGGCGGGTTAAT |
| | | CATTAACTACAAGGAACCCCTAGTGATGGAGTTGGCCACTCCCTCTCTGC |
| | | GCGCTCGCTCGCTCACTGAGGCCGGGCGACCAAAGGTCGCCCGACGCCC |
| | | GGGCTTTGCCCGGGCGGCCTCAGTGAGCGAGCGAGCGCGCCAGCTGGCG |
| | | TAATAGCGAAGAGGCCCGCACCGATCGCCCTTCCCAACAGTTGCGCAGCC |
| | | TGAATGGCGAATGGCGATTCCGTTGCAATGGCTGGCGGTAATATTGTTCT |
| | | GGATATTACCAGCAAGGCCGATAGTTTGAGTTCTTCTACTCAGGCAAGTG |
| | | ATGTTATTACTAATCAAAGAAGTATTGCGACAACGGTTAATTTGCGTGAT |
| | | GGACAGACTCTTTTACTCGGTGGCCTCACTGATTATAAAAACACTTCTCA |
| | | GGATTCTGGCGTACCGTTCCTGTCTAAAATCCCTTTAATCGGCCTCCTGTT |
| | | TAGCTCCCGCTCTGATTCTAACGAGGAAAGCACGTTATACGTGCTCGTCA |
| | | AAGCAACCATAGTACGCGCCCTGTAGCGGCGCATTAAGCGCGGCGGGTG |
| | | TGGTGGTTACGCGCAGCGTGACCGCTACACTTGCCAGCGCCCTAGCGCCC |
| | | GCTCCTTTCGCTTTCTTCCCTTCCTTTCTCGCCACGTTCGCCGGCTTTCCCC |
| | | GTCAAGCTCTAAATCGGGGGCTCCCTTTAGGGTTCCGATTTAGTGCTTTAC |
| | | GGCACCTCGACCCCAAAAAACTTGATTAGGGTGATGGTTCACGTAGTGGG |
| | | CCATCGCCCTGATAGACGGTTTTTCGCCCTTTGACGTTGGAGTCCACGTTC |
| | | TTTAATAGTGGACTCTTGTTCCAAACTGGAACAACACTCAACCCTATCTC |
| | | GGTCTATTCTTTTGATTTATAAGGGATTTTGCCGATTTCGGCCTATTGGTT |
| | | AAAAAATGAGCTGATTTAACAAAAATTTAACGCGAATTTTAACAAAATAT |
| | | TAACGTTTACAATTTAAATATTTGCTTATACAATCTTCCTGTTTTTGGGGC |
| | | TTTTCTGATTATCAACCGGGGTACATATGATTGACATGCTAGTTTTACGAT |
| | | TACCGTTCATCGATTCTCTTGTTTGCTCCAGACTCTCAGGCAATGACCTGA |
| | | TAGCCTTTGTAGAGACCTCTCAAAAATAGCTACCCTCTCCGGCATGAATT |
| | | TATCAGCTAGAACGGTTGAATATCATATTGATGGTGATTTGACTGTCTCC |
| | | GGCCTTTCTCACCCGTTTGAATCTTTACCTACACATTACTCAGGCATTGCA |
| | | TTTAAAATATATGAGGGTTCTAAAAATTTTTATCCTTGCGTTGAAATAAA |
| | | GGCTTCTCCCGCAAAAGTATTACAGGGTCATAATGTTTTTGGTACAACCG |
| | | ATTTAGCTTTATGCTCTGAGGCTTTATTGCTTAATTTTGCTAATTCTTTGCC |
| | | TTGCCTGTATGATTTATTGGATGTTGGAATCGCCTGATGCGGTATTTTCTC |
| | | CTTACGCATCTGTGCGGTATTTCACACCGCATATGGTGCACTCTCAGTACA |
| | | ATCTGCTCTGATGCCGCATAGTTAAGCCAGCCCCGACACCCGCCAACACC |
| | | CGCTGACGCGCCCTGACGGGCTTGTCTGCTCCCGGCATCCGCTTACAGAC |
| | | AAGCTGTGACCGTCTCCGGGAGCTGCATGTGTCAGAGGTTTTCACCGTCA |
| | | TCACCGAAACGCGCGAGACGAAAGGGCCTCGTGATACGCCTATTTTTATA |
| | | GGTTAATGTCATGATAATAATGGTTTCTTAGACGTCAGGTGGCACTTTTCG |
| | | GGGAAATGTGCGCGGAACCCCTATTTGTTTATTTTTCTAAATACATTCAAA |
| | | TATGTATCCGCTCATGAGACAATAACCCTGATAAATGCTTCAATAATATT |
| | | GAAAAAGGAAGAGTATGAGTATTCAACATTTCCGTGTCGCCCTTATTCCC |
| | | TTTTTTGCGGCATTTTGCCTTCCTGTTTTTGCTCACCCAGAAACGCTGGTG |
| | | AAAGTAAAAGATGCTGAAGATCAGTTGGGTGCACGAGTGGGTTACATCG |
| | | AACTGGATCTCAACAGCGGTAAGATCCTTGAGAGTTTTCGCCCCGAAGAA |
| | | CGTTTTCCAATGATGAGCACTTTTAAAGTTCTGCTATGTGGCGCGGTATTA |
| | | TCCCGTATTGACGCCGGGCAAGAGCAACTCGGTCGCCGCATACACTATTC |
| | | TCAGAATGACTTGGTTGAGTACTCACCAGTCACAGAAAAGCATCTTACGG |
| | | ATGGCATGACAGTAAGAGAATTATGCAGTGCTGCCATAACCATGAGTGAT |
| | | AACACTGCGGCCAACTTACTTCTGACAACGATCGGAGGACCGAAGGAGC |
| | | TAACCGCTTTTTTGCACAACATGGGGGATCATGTAACTCGCCTTGATCGTT |
| | | GGGAACCGGAGCTGAATGAAGCCATACCAAACGACGAGCGTGACACCAC |
| | | GATGCCTGTAGCAATGGCAACAACGTTGCGCAAACTATTAACTGGCGAAC |
| | | TACTTACTCTAGCTTCCCGGCAACAATTAATAGACTGGATGGAGGCGGAT |
| | | AAAGTTGCAGGACCACTTCTGCGCTCGGCCCTTCCGGCTGGCTGGTTTATT |
| | | GCTGATAAATCTGGAGCCGGTGAGCGTGGGTCTCGCGGTATCATTGCAGC |
| | | ACTGGGGCCAGATGGTAAGCCCTCCCGTATCGTAGTTATCTACACGACGG |
| | | GGAGTCAGGCAACTATGGATGAACGAAATAGACAGATCGCTGAGATAGG |
| | | TGCCTCACTGATTAAGCATTGGTAACTGTCAGACCAAGTTTACTCATATAT |
| | | ACTTTAGATTGATTTAAAACTTCATTTTTAATTTAAAAGGATCTAGGTGAA |
| | | GATCCTTTTTGATAATCTCATGACCAAAATCCCTTAACGTGAGTTTTCGTT |
| | | CCACTGAGCGTCAGACCCCGTAGAAAAGATCAAAGGATCTTCTTGAGATC |
| | | CTTTTTTTCTGCGCGTAATCTGCTGCTTGCAAACAAAAAAACCACCGCTAC |
| | | CAGCGGTGGTTTGTTTGCCGGATCAAGAGCTACCAACTCTTTTTCCGAAG |
| | | GTAACTGGCTTCAGCAGAGCGCAGATACCAAATACTGTCCTTCTAGTGTA |
| | | GCCGTAGTTAGGCCACCACTTCAAGAACTCTGTAGCACCGCCTACATACC |
| | | TCGCTCTGCTAATCCTGTTACCAGTGGCTGCTGCCAGTGGCGATAAGTCG |
| | | TGTCTTACCGGGTTGGACTCAAGACGATAGTTACCGGATAAGGCGCAGCG |
| | | GTCGGGCTGAACGGGGGGTTCGTGCACACAGCCCAGCTTGGAGCGAACG |
| | | ACCTACACCGAACTGAGATACCTACAGCGTGAGCTATGAGAAAGCGCCA |
| | | CGCTTCCCGAAGGGAGAAAGGCGGACAGGTATCCGGTAAGCGGCAGGGT |
| | | CGGAACAGGAGAGCGCACGAGGGAGCTTCCAGGGGGAAACGCCTGGTAT |
| | | CTTTATAGTCCTGTCGGGTTTCGCCACCTCTGACTTGAGCGTCGATTTTTG |
| | | TGATGCTCGTCAGGGGGGCGGAGCCTATGGAAAAACGCCAGCAACGCGG |
| | | CCTTTTTACGGTTCCTGGCCTTTTGCTGGCCTTTTGCTCACATGTTCTTTCC |
| | | TGCGTTATCCCCTGATTCTGTGGATAACCGTATTACCGCCTTTGAGTGAGC |
| | | TGATACCGCTCGCCGCAGCCGAACGACCGAGCGCAGCGAGTCAGTGAGC |
| | | GAGGAAGCGGAAGAGCGCCCAATACGCAAACCGCCTCTCCCCGCGCGTT |
| | | GGCCGATTCATTAATG |
| 163 | DNA sequence | GCCAAGCCTATGGCTCCTTCGCTCGCGTTAGGGCCTAGCCCAGGAGTCTT |
| | of murine | GCCTTCGTGGAAAACAGCACCCAAGGGCTCAGAACTTCTAGGGACCAGG |
| | FOXP3 1st | GGCTCTGGGGGACCCTTCCAAGGTCGGGACCTGCGAAGTGGGGCCCACA |
| | coding exon | CCTCTTCTTCCTTGAACCCCCTGCCACCATCCCAGCTGCAG |
| | included in AAV | |
| | #1331, 3209 and | |
| | 3213; modified | |
| | to be non- | |
| | cleavable for | |
| | TALEN, mT20, | |
| | mT22 and | |
| | mT23. | |

1.-27. (canceled)

28. A method of modifying a lymphocytic cell, the method comprising delivering to a lymphocytic cell a donor template comprising:
a) a first homology arm having homology to a sequence in a FOXP3 locus, AAVS1 locus, or TRAC locus in the lymphocytic cell;
b) a second homology arm having homology to a sequence in the same locus as the first homology arm;
c) a promoter; and
d) a sequence encoding FOXP3 or a functional derivative thereof,
wherein the promoter and the sequence encoding FOXP3 or a functional derivative thereof are located between the first homology arm and second homology arm.

29. The method of claim 28, further comprising delivering to the cell a DNA endonuclease or a nucleic acid encoding the DNA endonuclease.

30. The method of claim 29, further comprising delivering to the cell a gRNA comprising a spacer sequence that is complementary to the FOXP3 locus, AAVS1 locus, or TRAC locus.

31. The method of claim 30, wherein the gRNA comprises:
i) a spacer sequence from any one of SEQ ID NOs: 1-7, 15-20, 27-29, and 33-34 or a variant thereof having no more than 3 mismatches compared to any one of SEQ ID NOs: 1-7, 15-20, 27-29, and 33-34;
ii) a spacer sequence from any one of SEQ ID NOs: 1-7 or a variant thereof having no more than 3 mismatches compared to any one of SEQ ID NOs: 1-7; or
iii) a spacer sequence from any one of SEQ ID NOs: 2, 3, and 5 or a variant thereof having no more than 3 mismatches compared to any one of SEQ ID NOs: 2, 3, and 5.

32. The method of claim 28, wherein the first homology arm has homology to a sequence in the FOXP3 locus, and the second homology arm has homology to a sequence in the FOXP3 locus.

33. The method of claim 28, wherein:
a) the FOXP3 or functional derivative thereof is a wild-type human FOXP3;
b) the donor template is encoded in an adeno-associated virus (AAV) vector; and/or
c) the promoter is an MND promoter, PGK promoter, or E2F promoter.

34. The method of claim 28, wherein the sequence encoding FOXP3 or a functional derivative thereof is codon-optimized for expression in the cell.

35. The method of claim 34, wherein the sequence encoding FOXP3 or a functional derivative thereof is a FOXP3 cDNA sequence.

36. The method of claim 35, wherein the FOXP3 cDNA sequence comprises at least 90% sequence identity to the nucleic acid sequence of SEQ ID NO: 68.

37. The method of claim 28, wherein the donor template further comprises a sequence encoding a selectable marker, and the method further comprises separating cells expressing the selectable marker from cells that do not express the selectable marker.

38. A genetically modified lymphocytic cell made by the method of claim 28.

39. The genetically modified lymphocytic cell of claim 38, wherein the cell is a T cell.

40. The genetically modified lymphocytic cell of claim 39, wherein the cell is a FOXP3+ regulatory T cell.

41. The genetically modified lymphocytic cell of claim 38, wherein the sequence encoding a FOXP3 or a functional derivative thereof is a FOXP3 cDNA, wherein the promoter is an MND promoter.

42. A pharmaceutical composition comprising the genetically modified lymphocytic cell of claim 38 and a pharmaceutically acceptable excipient.

43. A lymphocytic cell comprising a nucleic acid sequence comprising a promoter operably linked to a FOXP3 cDNA sequence encoding FOPX3, wherein the promoter and FOXP3 cDNA sequence are located in a FOXP3 locus, AAVS1 locus, or TRAC locus in the lymphocytic cell.

44. A method of treating a disease or a condition in a subject, the method comprising administering to the subject the cell of claim 43.

45. The method of claim 44, wherein the disease or condition is an inflammatory disease, autoimmune disease, or a condition associated with a solid organ transplant.

46. The method of claim 44, wherein the disease is IPEX syndrome, Graft-versus-Host disease (GvHD), systemic lupus, scleroderma, hemolytic anemia, vasculitis, type I diabetes, Graves' disease, inflammatory bowel disease, rheumatoid arthritis, multiple sclerosis, Goodpasture's syndrome, myopathy, severe combined immunodeficiency, DiGeorge syndrome, Hyperimmunoglobulin E syndrome, Common variable immunodeficiency, Chronic granulomatous disease, Wiskott-Aldrich syndrome, Autoimmune lymphoproliferative syndrome, Hyper IgM syndrome, Leukocyte adhesion deficiency, NF-kB Essential Modifier (NEMO) Mutations, Selective immunoglobulin A deficiency, X-linked agammaglobulinemia, X-linked lymphoproliferative disease, or Ataxia-telangiectasia.

47. A nucleic acid comprising:
a) a first homology arm having homology to a sequence in a FOXP3 locus, AAVS1 locus, or TRAC locus in a lymphocytic cell;
b) a second homology arm having homology to a sequence in the same locus as the first homology arm;
c) a promoter; and
d) a sequence encoding FOXP3 or a functional derivative thereof,
wherein the promoter and the sequence encoding FOXP3 or a functional derivative thereof are located between the first homology arm and second homology arm.


SEQUENCE LISTING (163 sequences)

SEQ ID NO: 1 - DNA, 20 bp, Artificial Sequence
  T1 spacer targeting human FOXP3
ttccagggccgagatcttcg

SEQ ID NO: 2 - DNA, 20 bp, Artificial Sequence
  T3 spacer targeting human FOXP3
cgcctcgaagatctcggccc

SEQ ID NO: 3 - DNA, 20 bp, Artificial Sequence
  T4 spacer targeting human FOXP3
tcgaagatctcggccctgga

SEQ ID NO: 4 - DNA, 20 bp, Artificial Sequence
  T7 spacer targeting human FOXP3
ggccctggaaggttccccct

SEQ ID NO: 5 - DNA, 20 bp, Artificial Sequence
  T9 spacer targeting human FOXP3
tccagctgggcgaggctcct

SEQ ID NO: 6 - DNA, 20 bp, Artificial Sequence
  T18 spacer targeting human FOXP3
tcagacctgctgggggcccg

SEQ ID NO: 7 - DNA, 20 bp, Artificial Sequence
  R1 spacer targeting human FOXP3
gagccccgcctcgaagatct

SEQ ID NO: 8 - DNA, 3 bp, Artificial Sequence
  PAM sequence
agg

SEQ ID NO: 9 - DNA, 3 bp, Artificial Sequence
  PAM sequence
tgg

SEQ ID NO: 10 - DNA, 3 bp, Artificial Sequence
  PAM sequence
agg

SEQ ID NO: 11 - DNA, 3 bp, Artificial Sequence
  PAM sequence
ggg

SEQ ID NO: 12 - DNA, 3 bp, Artificial Sequence
  PAM sequence
ggg

SEQ ID NO: 13 - DNA, 3 bp, Artificial Sequence
  PAM sequence
ggg

SEQ ID NO: 14 - DNA, 3 bp, Artificial Sequence
  PAM sequence
cgg

SEQ ID NO: 15 - DNA, 20 bp, Artificial Sequence
  P1 spacer targeting human AAVS1
attcccagggccggttaatg

SEQ ID NO: 16 - DNA, 20 bp, Artificial Sequence
  P3 spacer targeting human AAVS1
gtcccctccaccccacagtg

SEQ ID NO: 17 - DNA, 20 bp, Artificial Sequence
  P4 spacer targeting human AAVS1
accccacagtggggccacta

SEQ ID NO: 18 - DNA, 20 bp, Artificial Sequence
  N1 spacer targeting human AAVS1
cctctaaggtttgcttacga

SEQ ID NO: 19 - DNA, 20 bp, Artificial Sequence
  N2 spacer targeting human AAVS1
tataaggtggtcccagctcg

SEQ ID NO: 20 - DNA, 20 bp, Artificial Sequence
  N3 spacer targeting human AAVS1
ccatcgtaagcaaaccttag

SEQ ID NO: 21 - DNA, 3 bp, Artificial Sequence
  PAM sequence
tgg

SEQ ID NO: 22 - DNA, 3 bp, Artificial Sequence
  PAM sequence
ggg

SEQ ID NO: 23 - DNA, 3 bp, Artificial Sequence
  PAM sequence
ggg

SEQ ID NO: 24 - DNA, 3 bp, Artificial Sequence
  PAM sequence
tgg

SEQ ID NO: 25 - DNA, 3 bp, Artificial Sequence
  PAM sequence
ggg

SEQ ID NO: 26 - DNA, 3 bp, Artificial Sequence
  PAM sequence
agg

SEQ ID NO: 27 - DNA, 20 bp, Artificial Sequence
  mT20 spacer target murine FOXP3
gactcctggggatgggccaa

SEQ ID NO: 28 - DNA, 20 bp, Artificial Sequence
  mT22 spacer target murine FOXP3
ttggcccttggcccatcccc

SEQ ID NO: 29 - DNA, 20 bp, Artificial Sequence
  mT23 spacer target murine FOXP3
ccagcttggcaagactcctg

SEQ ID NO: 30 - DNA, 3 bp, Artificial Sequence
  PAM sequence
ggg

SEQ ID NO: 31 - DNA, 3 bp, Artificial Sequence
  PAM sequence
agg

SEQ ID NO: 32 - DNA, 3 bp, Artificial Sequence
  PAM sequence
ggg

SEQ ID NO: 33 - DNA, 20 bp, Artificial Sequence
  human TRAC spacer sequence G2
acaaaactgtgctagacatg

SEQ ID NO: 34 - DNA, 18 bp, Artificial Sequence
  human TRAC spacer sequence G4
tcaagagcaacagtgctg

SEQ ID NO: 35 - DNA, 3 bp, Artificial Sequence
  PAM sequence
agg

SEQ ID NO: 36 - DNA, 3 bp, Artificial Sequence
  PAM sequence
tgg

SEQ ID NO: 37 - DNA, 2190 bp, Artificial Sequence
  FOXP3cDNA-P2A-LNGFR
gccaccatgcctaatcctcggcctggaaagcctagcgctccttctcttgctctgggaccttctcctggcgcctctccatcttggagagccgctcctaaagccagcgatctgctgggagctagaggacctggcggcacatttcagggcagagatcttagaggcggagcccacgctagctcctccagccttaatcctatgcctcctagccagctccagctgcctacactgcctctggttatggtggctcctagcggagctagactgggccctctgcctcatctgcaagctctgctgcaggacagaccccacttcatgcaccagctgagcaccgtggatgcccacgcaagaacacctgtgctgcaggttcaccctctggaatccccagccatgatcagcctgacacctccaacaacagccaccggcgtgttcagcctgaaagccagacctggactgcctcctggcatcaatgtggccagcctggaatgggtgtccagagaacctgctctgctgtgcacattccccaatccaagcgctcccagaaaggacagcacactgtctgccgtgcctcagagcagctatcccctgcttgctaacggcgtgtgcaagtggcctggatgcgagaaggtgttcgaggaacccgaggacttcctgaagcactgccaggccgatcatctgctggacgagaaaggcagagcccagtgtctgctccagcgcgagatggtgcagtctctggaacagcagctggtcctggaaaaagaaaagctgagcgccatgcaggcccacctggccggaaaaatggccctgacaaaggccagcagcgtggcctcttctgataagggcagctgctgcattgtggccgctggatctcagggacctgtggttcctgcttggagcggacctagagaggcccctgattctctgtttgccgtgcggagacacctgtggggctctcacggcaactctactttccccgagttcctgcacaacatggactacttcaagttccacaacatgcggcctccattcacctacgccacactgatcagatgggccattctggaagcccctgagaagcagagaaccctgaacgagatctaccactggtttacccggatgttcgccttcttccggaatcaccctgccacctggaagaacgccatccggcacaatctgagcctgcacaagtgcttcgtgcgcgtggaatctgagaaaggcgccgtgtggacagtggacgagctggaattcagaaagaagagaagccagcggcctagccggtgcagcaatcctacacctggacctggaagcggagcgactaacttcagcctgctgaagcaggccggagatgtggaggaaaaccctggaccgatgggggcaggtgccaccggacgagccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggtga

SEQ ID NO: 38 - DNA, 2189 bp, Artificial Sequence
  LNGFR-P2A-FOXP3cDNA
ccaccatgggggcaggtgccaccggacgagccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggggaagcggagcgactaacttcagcctgctgaagcaggccggagatgtggaggaaaaccctggaccgatgcctaatcctcggcctggaaagcctagcgctccttctcttgctctgggaccttctcctggcgcctctccatcttggagagccgctcctaaagccagcgatctgctgggagctagaggacctggcggcacatttcagggcagagatcttagaggcggagcccacgctagctcctccagccttaatcctatgcctcctagccagctccagctgcctacactgcctctggttatggtggctcctagcggagctagactgggccctctgcctcatctgcaagctctgctgcaggacagaccccacttcatgcaccagctgagcaccgtggatgcccacgcaagaacacctgtgctgcaggttcaccctctggaatccccagccatgatcagcctgacacctccaacaacagccaccggcgtgttcagcctgaaagccagacctggactgcctcctggcatcaatgtggccagcctggaatgggtgtccagagaacctgctctgctgtgcacattccccaatccaagcgctcccagaaaggacagcacactgtctgccgtgcctcagagcagctatcccctgcttgctaacggcgtgtgcaagtggcctggatgcgagaaggtgttcgaggaacccgaggacttcctgaagcactgccaggccgatcatctgctggacgagaaaggcagagcccagtgtctgctccagcgcgagatggtgcagtctctggaacagcagctggtcctggaaaaagaaaagctgagcgccatgcaggcccacctggccggaaaaatggccctgacaaaggccagcagcgtggcctcttctgataagggcagctgctgcattgtggccgctggatctcagggacctgtggttcctgcttggagcggacctagagaggcccctgattctctgtttgccgtgcggagacacctgtggggctctcacggcaactctactttccccgagttcctgcacaacatggactacttcaagttccacaacatgcggcctccattcacctacgccacactgatcagatgggccattctggaagcccctgagaagcagagaaccctgaacgagatctaccactggtttacccggatgttcgccttcttccggaatcaccctgccacctggaagaacgccatccggcacaatctgagcctgcacaagtgcttcgtgcgcgtggaatctgagaaaggcgccgtgtggacagtggacgagctggaattcagaaagaagagaagccagcggcctagccggtgcagcaatcctacacctggaccttga

SEQ ID NO: 39 - DNA, 3261 bp, Artificial Sequence
  FOXP3cDNA-microDISC nucleotide sequence
atgcctaatcctcggcctggaaagcctagcgctccttctcttgctctgggaccttctcctggcgcctctccatcttggagagccgctcctaaagccagcgatctgctgggagctagaggacctggcggcacatttcagggcagagatcttagaggcggagcccacgctagctcctccagccttaatcctatgcctcctagccagctccagctgcctacactgcctctggttatggtggctcctagcggagctagactgggccctctgcctcatctgcaagctctgctgcaggacagaccccacttcatgcaccagctgagcaccgtggatgcccacgcaagaacacctgtgctgcaggttcaccctctggaatccccagccatgatcagcctgacacctccaacaacagccaccggcgtgttcagcctgaaagccagacctggactgcctcctggcatcaatgtggccagcctggaatgggtgtccagagaacctgctctgctgtgcacattccccaatccaagcgctcccagaaaggacagcacactgtctgccgtgcctcagagcagctatcccctgcttgctaacggcgtgtgcaagtggcctggatgcgagaaggtgttcgaggaacccgaggacttcctgaagcactgccaggccgatcatctgctggacgagaaaggcagagcccagtgtctgctccagcgcgagatggtgcagtctctggaacagcagctggtcctggaaaaagaaaagctgagcgccatgcaggcccacctggccggaaaaatggccctgacaaaggccagcagcgtggcctcttctgataagggcagctgctgcattgtggccgctggatctcagggacctgtggttcctgcttggagcggacctagagaggcccctgattctctgtttgccgtgcggagacacctgtggggctctcacggcaactctactttccccgagttcctgcacaacatggactacttcaagttccacaacatgcggcctccattcacctacgccacactgatcagatgggccattctggaagcccctgagaagcagagaaccctgaacgagatctaccactggtttacccggatgttcgccttcttccggaatcaccctgccacctggaagaacgccatccggcacaatctgagcctgcacaagtgcttcgtgcgcgtggaatctgagaaaggcgccgtgtggacagtggacgagctggaattcagaaagaagagaagccagcggcctagccggtgcagcaatcctacacctggacctggaagcggagcgactaacttcagcctgcttaagcaggccggagatgtggaggaaaaccctggaccgatgcctctgggcctgctgtggctgggcctggccctgctgggcgccctgcacgcccaggccggcgtgcaggtggagacaatctccccaggcgacggacgcacattccctaagcggggccagacctgcgtggtgcactatacaggcatgctggaggatggcaagaagtttgacagctcccgggatagaaacaagccattcaagtttatgctgggcaagcaggaagtgatcagaggctgggaggagggcgtggcccagatgtctgtgggccagagggccaagctgaccatcagcccagactacgcctatggagcaacaggccacccaggaatcatcccacctcacgccaccctggtgttcgatgtggagctgctgaagctgggcgagggagggtcacctggatccaacacatcaaaagagaacccctttctgttcgcattggaggccgtagtcatatctgttggatccatgggacttattatctccctgttgtgtgtgtacttctggctggaacggactatgcccaggatccccacgctcaagaatctggaagatctcgtcacagaataccatggtaatttcagcgcctggagcggagtctctaagggtctggccgaatccctccaacccgattattctgaacggttgtgcctcgtatccgaaataccaccaaaaggcggggctctgggtgagggcccaggggcgagtccgtgcaatcaacacagcccgtattgggcccctccttgttatacgttgaagcccgaaactggaagcggagctactaacttcagcctgctgaagcaggctggagacgtggaggagaaccctggacctatggcactgcccgtgaccgccctgctgctgcctctggccctgctgctgcacgcagcccggcctatcctgtggcacgagatgtggcacgagggcctggaggaggccagcaggctgtattttggcgagcgcaacgtgaagggcatgttcgaggtgctggagcctctgcacgccatgatggagagaggcccacagaccctgaaggagacatcctttaaccaggcctatggacgggacctgatggaggcacaggagtggtgcagaaagtacatgaagtctggcaatgtgaaggacctgctgcaggcctgggatctgtactatcacgtgtttcggagaatctccaagccagcagctctcggcaaagacacgattccgtggcttgggcatctgctcgttgggctgagcggtgcgtttggtttcatcatcttggtctatctcttgatcaattgcagaaatacaggcccttggctgaaaaaagtgctcaagtgtaatacccccgacccaagcaagttcttctcccagctttcttcagagcatggaggcgatgtgcagaaatggctctcttcaccttttccctcctcaagcttctccccgggagggctggcgcccgagatttcacctcttgaggtacttgaacgagacaaggttacccaacttctccttcaacaggataaggtacccgaacctgcgagccttagcttgaatacagacgcttatctctcactgcaggaactgcaaggatctggtgctactaatttttctcttttgaagcaagctggagatgttgaagagaaccccggtccggagatgtggcatgagggtctggaagaagcgtctcgactgtactttggtgagcgcaatgtgaagggcatgtttgaagtcctcgaaccccttcatgccatgatggaacgcggaccccagaccttgaaggagacaagttttaaccaagcttacggaagagacctgatggaagcccaggaatggtgcaggaaatacatgaaaagcgggaatgtgaaggacttgctccaagcgtgggacctgtactatcatgtctttaggcgcattagtaagtga

SEQ ID NO: 40 - DNA, 4080 bp, Artificial Sequence
  FOXP3cDNA-LNGFRe-microDISC nucleotide sequence
atgcctaatcctcggcctggaaagcctagcgctccttctcttgctctgggaccttctcctggcgcctctccatcttggagagccgctcctaaagccagcgatctgctgggagctagaggacctggcggcacatttcagggcagagatcttagaggcggagcccacgctagctcctccagccttaatcctatgcctcctagccagctccagctgcctacactgcctctggttatggtggctcctagcggagctagactgggccctctgcctcatctgcaagctctgctgcaggacagaccccacttcatgcaccagctgagcaccgtggatgcccacgcaagaacacctgtgctgcaggttcaccctctggaatccccagccatgatcagcctgacacctccaacaacagccaccggcgtgttcagcctgaaagccagacctggactgcctcctggcatcaatgtggccagcctggaatgggtgtccagagaacctgctctgctgtgcacattccccaatccaagcgctcccagaaaggacagcacactgtctgccgtgcctcagagcagctatcccctgcttgctaacggcgtgtgcaagtggcctggatgcgagaaggtgttcgaggaacccgaggacttcctgaagcactgccaggccgatcatctgctggacgagaaaggcagagcccagtgtctgctccagcgcgagatggtgcagtctctggaacagcagctggtcctggaaaaagaaaagctgagcgccatgcaggcccacctggccggaaaaatggccctgacaaaggccagcagcgtggcctcttctgataagggcagctgctgcattgtggccgctggatctcagggacctgtggttcctgcttggagcggacctagagaggcccctgattctctgtttgccgtgcggagacacctgtggggctctcacggcaactctactttccccgagttcctgcacaacatggactacttcaagttccacaacatgcggcctccattcacctacgccacactgatcagatgggccattctggaagcccctgagaagcagagaaccctgaacgagatctaccactggtttacccggatgttcgccttcttccggaatcaccctgccacctggaagaacgccatccggcacaatctgagcctgcacaagtgcttcgtgcgcgtggaatctgagaaaggcgccgtgtggacagtggacgagctggaattcagaaagaagagaagccagcggcctagccggtgcagcaatcctacacctggacctggaagcggagcgactaacttcagcctgcttaagcaggccggagatgtggaggaaaaccctggaccgatgcctctgggcctgctgtggctgggcctggccctgctgggcgccctgcacgcccaggccatgggggcaggtgccaccggacgagccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggggcgtgcaggtggagacaatctccccaggcgacggacgcacattccctaagcggggccagacctgcgtggtgcactatacaggcatgctggaggatggcaagaagtttgacagctcccgggatagaaacaagccattcaagtttatgctgggcaagcaggaagtgatcagaggctgggaggagggcgtggcccagatgtctgtgggccagagggccaagctgaccatcagcccagactacgcctatggagcaacaggccacccaggaatcatcccacctcacgccaccctggtgttcgatgtggagctgctgaagctgggcgagggagggtcacctggatccaacacatcaaaagagaacccctttctgttcgcattggaggccgtagtcatatctgttggatccatgggacttattatctccctgttgtgtgtgtacttctggctggaacggactatgcccaggatccccacgctcaagaatctggaagatctcgtcacagaataccatggtaatttcagcgcctggagcggagtctctaagggtctggccgaatccctccaacccgattattctgaacggttgtgcctcgtatccgaaataccaccaaaaggcggggctctgggtgagggcccaggggcgagtccgtgcaatcaacacagcccgtattgggcccctccttgttatacgttgaagcccgaaactggaagcggagctactaacttcagcctgctgaagcaggctggagacgtggaggagaaccctggacctatggcactgcccgtgaccgccctgctgctgcctctggccctgctgctgcacgcagcccggcctatcctgtggcacgagatgtggcacgagggcctggaggaggccagcaggctgtattttggcgagcgcaacgtgaagggcatgttcgaggtgctggagcctctgcacgccatgatggagagaggcccacagaccctgaaggagacatcctttaaccaggcctatggacgggacctgatggaggcacaggagtggtgcagaaagtacatgaagtctggcaatgtgaaggacctgctgcaggcctgggatctgtactatcacgtgtttcggagaatctccaagccagcagctctcggcaaagacacgattccgtggcttgggcatctgctcgttgggctgagcggtgcgtttggtttcatcatcttggtctatctcttgatcaattgcagaaatacaggcccttggctgaaaaaagtgctcaagtgtaatacccccgacccaagcaagttcttctcccagctttcttcagagcatggaggcgatgtgcagaaatggctctcttcaccttttccctcctcaagcttctccccgggagggctggcgcccgagatttcacctcttgaggtacttgaacgagacaaggttacccaacttctccttcaacaggataaggtacccgaacctgcgagccttagcttgaatacagacgcttatctctcactgcaggaactgcaaggatctggtgctactaatttttctcttttgaagcaagctggagatgttgaagagaaccccggtccggagatgtggcatgagggtctggaagaagcgtctcgactgtactttggtgagcgcaatgtgaagggcatgtttgaagtcctcgaaccccttcatgccatgatggaacgcggaccccagaccttgaaggagacaagttttaaccaagcttacggaagagacctgatggaagcccaggaatggtgcaggaaatacatgaaaagcgggaatgtgaaggacttgctccaagcgtgggacctgtactatcatgtctttaggcgcattagtaag

SEQ ID NO: 41 - DNA, 3258 bp, Artificial Sequence
  microDISC-FOXP3cDNA nucleotide sequence
atgcctctgggcctgctgtggctgggcctggccctgctgggcgccctgcacgcccaggccggcgtgcaggtggagacaatctccccaggcgacggacgcacattccctaagcggggccagacctgcgtggtgcactatacaggcatgctggaggatggcaagaagtttgacagctcccgggatagaaacaagccattcaagtttatgctgggcaagcaggaagtgatcagaggctgggaggagggcgtggcccagatgtctgtgggccagagggccaagctgaccatcagcccagactacgcctatggagcaacaggccacccaggaatcatcccacctcacgccaccctggtgttcgatgtggagctgctgaagctgggcgagggagggtcacctggatccaacacatcaaaagagaacccctttctgttcgcattggaggccgtagtcatatctgttggatccatgggacttattatctccctgttgtgtgtgtacttctggctggaacggactatgcccaggatccccacgctcaagaatctggaagatctcgtcacagaataccatggtaatttcagcgcctggagcggagtctctaagggtctggccgaatccctccaacccgattattctgaacggttgtgcctcgtatccgaaataccaccaaaaggcggggctctgggtgagggcccaggggcgagtccgtgcaatcaacacagcccgtattgggcccctccttgttatacgttgaagcccgaaactggaagcggagctactaacttcagcctgctgaagcaggctggagacgtggaggagaaccctggacctatggcactgcccgtgaccgccctgctgctgcctctggccctgctgctgcacgcagcccggcctatcctgtggcacgagatgtggcacgagggcctggaggaggccagcaggctgtattttggcgagcgcaacgtgaagggcatgttcgaggtgctggagcctctgcacgccatgatggagagaggcccacagaccctgaaggagacatcctttaaccaggcctatggacgggacctgatggaggcacaggagtggtgcagaaagtacatgaagtctggcaatgtgaaggacctgctgcaggcctgggatctgtactatcacgtgtttcggagaatctccaagccagcagctctcggcaaagacacgattccgtggcttgggcatctgctcgttgggctgagcggtgcgtttggtttcatcatcttggtctatctcttgatcaattgcagaaatacaggcccttggctgaaaaaagtgctcaagtgtaatacccccgacccaagcaagttcttctcccagctttcttcagagcatggaggcgatgtgcagaaatggctctcttcaccttttccctcctcaagcttctccccgggagggctggcgcccgagatttcacctcttgaggtacttgaacgagacaaggttacccaacttctccttcaacaggataaggtacccgaacctgcgagccttagcttgaatacagacgcttatctctcactgcaggaactgcaaggatctggtgctactaatttttctcttttgaagcaagctggagatgttgaagagaaccccggtccggagatgtggcatgagggtctggaagaagcgtctcgactgtactttggtgagcgcaatgtgaagggcatgtttgaagtcctcgaaccccttcatgccatgatggaacgcggaccccagaccttgaaggagacaagttttaaccaagcttacggaagagacctgatggaagcccaggaatggtgcaggaaatacatgaaaagcgggaatgtgaaggacttgctccaagcgtgggacctgtactatcatgtctttaggcgcattagtaagggaagcggagcgactaacttcagcctgcttaagcaggccggagatgtggaggaaaaccctggaccgatgcctaatcctcggcctggaaagcctagcgctccttctcttgctctgggaccttctcctggcgcctctccatcttggagagccgctcctaaagccagcgatctgctgggagctagaggacctggcggcacatttcagggcagagatcttagaggcggagcccacgctagctcctccagccttaatcctatgcctcctagccagctccagctgcctacactgcctctggttatggtggctcctagcggagctagactgggccctctgcctcatctgcaagctctgctgcaggacagaccccacttcatgcaccagctgagcaccgtggatgcccacgcaagaacacctgtgctgcaggttcaccctctggaatccccagccatgatcagcctgacacctccaacaacagccaccggcgtgttcagcctgaaagccagacctggactgcctcctggcatcaatgtggccagcctggaatgggtgtccagagaacctgctctgctgtgcacattccccaatccaagcgctcccagaaaggacagcacactgtctgccgtgcctcagagcagctatcccctgcttgctaacggcgtgtgcaagtggcctggatgcgagaaggtgttcgaggaacccgaggacttcctgaagcactgccaggccgatcatctgctggacgagaaaggcagagcccagtgtctgctccagcgcgagatggtgcagtctctggaacagcagctggtcctggaaaaagaaaagctgagcgccatgcaggcccacctggccggaaaaatggccctgacaaaggccagcagcgtggcctcttctgataagggcagctgctgcattgtggccgctggatctcagggacctgtggttcctgcttggagcggacctagagaggcccctgattctctgtttgccgtgcggagacacctgtggggctctcacggcaactctactttccccgagttcctgcacaacatggactacttcaagttccacaacatgcggcctccattcacctacgccacactgatcagatgggccattctggaagcccctgagaagcagagaaccctgaacgagatctaccactggtttacccggatgttcgccttcttccggaatcaccctgccacctggaagaacgccatccggcacaatctgagcctgcacaagtgcttcgtgcgcgtggaatctgagaaaggcgccgtgtggacagtggacgagctggaattcagaaagaagagaagccagcggcctagccggtgcagcaatcctacacctggacct

SEQ ID NO: 42 - DNA, 4083 bp, Artificial Sequence
  LNGFRe-microDISC -FOXP3cDNA nucleotide sequence
atgcctctgggcctgctgtggctgggcctggccctgctgggcgccctgcacgcccaggccatgggggcaggtgccaccggacgagccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggggcgtgcaggtggagacaatctccccaggcgacggacgcacattccctaagcggggccagacctgcgtggtgcactatacaggcatgctggaggatggcaagaagtttgacagctcccgggatagaaacaagccattcaagtttatgctgggcaagcaggaagtgatcagaggctgggaggagggcgtggcccagatgtctgtgggccagagggccaagctgaccatcagcccagactacgcctatggagcaacaggccacccaggaatcatcccacctcacgccaccctggtgttcgatgtggagctgctgaagctgggcgagggagggtcacctggatccaacacatcaaaagagaacccctttctgttcgcattggaggccgtagtcatatctgttggatccatgggacttattatctccctgttgtgtgtgtacttctggctggaacggactatgcccaggatccccacgctcaagaatctggaagatctcgtcacagaataccatggtaatttcagcgcctggagcggagtctctaagggtctggccgaatccctccaacccgattattctgaacggttgtgcctcgtatccgaaataccaccaaaaggcggggctctgggtgagggcccaggggcgagtccgtgcaatcaacacagcccgtattgggcccctccttgttatacgttgaagcccgaaactggaagcggagctactaacttcagcctgctgaagcaggctggagacgtggaggagaaccctggacctatggcactgcccgtgaccgccctgctgctgcctctggccctgctgctgcacgcagcccggcctatcctgtggcacgagatgtggcacgagggcctggaggaggccagcaggctgtattttggcgagcgcaacgtgaagggcatgttcgaggtgctggagcctctgcacgccatgatggagagaggcccacagaccctgaaggagacatcctttaaccaggcctatggacgggacctgatggaggcacaggagtggtgcagaaagtacatgaagtctggcaatgtgaaggacctgctgcaggcctgggatctgtactatcacgtgtttcggagaatctccaagccagcagctctcggcaaagacacgattccgtggcttgggcatctgctcgttgggctgagcggtgcgtttggtttcatcatcttggtctatctcttgatcaattgcagaaatacaggcccttggctgaaaaaagtgctcaagtgtaatacccccgacccaagcaagttcttctcccagctttcttcagagcatggaggcgatgtgcagaaatggctctcttcaccttttccctcctcaagcttctccccgggagggctggcgcccgagatttcacctcttgaggtacttgaacgagacaaggttacccaacttctccttcaacaggataaggtacccgaacctgcgagccttagcttgaatacagacgcttatctctcactgcaggaactgcaaggatctggtgctactaatttttctcttttgaagcaagctggagatgttgaagagaaccccggtccggagatgtggcatgagggtctggaagaagcgtctcgactgtactttggtgagcgcaatgtgaagggcatgtttgaagtcctcgaaccccttcatgccatgatggaacgcggaccccagaccttgaaggagacaagttttaaccaagcttacggaagagacctgatggaagcccaggaatggtgcaggaaatacatgaaaagcgggaatgtgaaggacttgctccaagcgtgggacctgtactatcatgtctttaggcgcattagtaagggaagcggagcgactaacttcagcctgcttaagcaggccggagatgtggaggaaaaccctggaccgatgcctaatcctcggcctggaaagcctagcgctccttctcttgctctgggaccttctcctggcgcctctccatcttggagagccgctcctaaagccagcgatctgctgggagctagaggacctggcggcacatttcagggcagagatcttagaggcggagcccacgctagctcctccagccttaatcctatgcctcctagccagctccagctgcctacactgcctctggttatggtggctcctagcggagctagactgggccctctgcctcatctgcaagctctgctgcaggacagaccccacttcatgcaccagctgagcaccgtggatgcccacgcaagaacacctgtgctgcaggttcaccctctggaatccccagccatgatcagcctgacacctccaacaacagccaccggcgtgttcagcctgaaagccagacctggactgcctcctggcatcaatgtggccagcctggaatgggtgtccagagaacctgctctgctgtgcacattccccaatccaagcgctcccagaaaggacagcacactgtctgccgtgcctcagagcagctatcccctgcttgctaacggcgtgtgcaagtggcctggatgcgagaaggtgttcgaggaacccgaggacttcctgaagcactgccaggccgatcatctgctggacgagaaaggcagagcccagtgtctgctccagcgcgagatggtgcagtctctggaacagcagctggtcctggaaaaagaaaagctgagcgccatgcaggcccacctggccggaaaaatggccctgacaaaggccagcagcgtggcctcttctgataagggcagctgctgcattgtggccgctggatctcagggacctgtggttcctgcttggagcggacctagagaggcccctgattctctgtttgccgtgcggagacacctgtggggctctcacggcaactctactttccccgagttcctgcacaacatggactacttcaagttccacaacatgcggcctccattcacctacgccacactgatcagatgggccattctggaagcccctgagaagcagagaaccctgaacgagatctaccactggtttacccggatgttcgccttcttccggaatcaccctgccacctggaagaacgccatccggcacaatctgagcctgcacaagtgcttcgtgcgcgtggaatctgagaaaggcgccgtgtggacagtggacgagctggaattcagaaagaagagaagccagcggcctagccggtgcagcaatcctacacctggaccttga

SEQ ID NO: 43 - DNA, 2463 bp, Artificial Sequence
  DISC nucleotide sequence
atgcctctgggcctgctgtggctgggcctggccctgctgggcgccctgcacgcccaggccggcgtgcaggtggagacaatctccccaggcgacggacgcacattccctaagcggggccagacctgcgtggtgcactatacaggcatgctggaggatggcaagaagtttgacagctcccgggatagaaacaagccattcaagtttatgctgggcaagcaggaagtgatcagaggctgggaggagggcgtggcccagatgtctgtgggccagagggccaagctgaccatcagcccagactacgcctatggagcaacaggccacccaggaatcatcccacctcacgccaccctggtgttcgatgtggagctgctgaagctgggcgagggagggtcacctggatccaacacatcaaaagagaacccctttctgttcgcattggaggccgtagtcatatctgttggatccatgggacttattatctccctgttgtgtgtgtacttctggctggaacggactatgcccaggatccccacgctcaagaatctggaagatctcgtcacagaataccatggtaatttcagcgcctggagcggagtctctaagggtctggccgaatccctccaacccgattattctgaacggttgtgcctcgtatccgaaataccaccaaaaggcggggctctgggtgagggcccaggggcgagtccgtgcaatcaacacagcccgtattgggcccctccttgttatacgttgaagcccgaaactggaagcggagctactaacttcagcctgctgaagcaggctggagacgtggaggagaaccctggacctatggcactgcccgtgaccgccctgctgctgcctctggccctgctgctgcacgcagcccggcctatcctgtggcacgagatgtggcacgagggcctggaggaggccagcaggctgtattttggcgagcgcaacgtgaagggcatgttcgaggtgctggagcctctgcacgccatgatggagagaggcccacagaccctgaaggagacatcctttaaccaggcctatggacgggacctgatggaggcacaggagtggtgcagaaagtacatgaagtctggcaatgtgaaggacctgctgcaggcctgggatctgtactatcacgtgtttcggagaatctccaagccagcagctctcggcaaagacacgattccgtggcttgggcatctgctcgttgggctgagcggtgcgtttggtttcatcatcttggtctatctcttgatcaattgcagaaatacaggcccttggctgaaaaaagtgctcaagtgtaatacccccgacccaagcaagttcttctcccagctttcttcagagcatggaggcgatgtgcagaaatggctctcttcaccttttccctcctcaagcttctccccgggagggctggcgcccgagatttcacctcttgaggtacttgaacgagacaaggttacccaacttctccttcaacaggataaggtacccgaacctgcgagccttagctccaaccactctcttacgagctgcttcaccaatcagggatacttctttttccaccttcccgatgcgctggaaatcgaagcttgtcaagtttactttacctatgatccatatagcgaggaagatcccgacgaaggagtcgccggtgcgcccacgggttcctcaccccaacctctccagcctctctcaggagaagatgatgcttattgcacttttcccagtagagacgatctcctcctcttttctccatctcttttggggggaccttcccccccttctacggcacctggcgggtctggtgctggcgaggagcggatgccgccgtccctccaggagcgagtaccacgagattgggatccccagccacttggaccccccacccccggcgtacctgaccttgtcgattttcaacctccccctgaattggtgctgcgagaggctggggaggaagttccggacgctgggccgagggagggcgtgtcctttccatggagtaggcctccaggtcaaggcgagtttagggctctcaacgcgcggctgccgttgaatacagacgcttatctctcactgcaggaactgcaaggtcaggacccaacacatcttgtaggatctggtgctactaatttttctcttttgaagcaagctggagatgttgaagagaaccccggtccggagatgtggcatgagggtctggaagaagcgtctcgactgtactttggtgagcgcaatgtgaagggcatgtttgaagtcctcgaaccccttcatgccatgatggaacgcggaccccagaccttgaaggagacaagttttaaccaagcttacggaagagacctgatggaagcccaggaatggtgcaggaaatacatgaaaagcgggaatgtgaaggacttgctccaagcgtgggacctgtactatcatgtctttaggcgcattagtaag

SEQ ID NO: 44 - DNA, 1899 bp, Artificial Sequence
  microDISC nucleotide sequence
atgcctctgggcctgctgtggctgggcctggccctgctgggcgccctgcacgcccaggccggcgtgcaggtggagacaatctccccaggcgacggacgcacattccctaagcggggccagacctgcgtggtgcactatacaggcatgctggaggatggcaagaagtttgacagctcccgggatagaaacaagccattcaagtttatgctgggcaagcaggaagtgatcagaggctgggaggagggcgtggcccagatgtctgtgggccagagggccaagctgaccatcagcccagactacgcctatggagcaacaggccacccaggaatcatcccacctcacgccaccctggtgttcgatgtggagctgctgaagctgggcgagggagggtcacctggatccaacacatcaaaagagaacccctttctgttcgcattggaggccgtagtcatatctgttggatccatgggacttattatctccctgttgtgtgtgtacttctggctggaacggactatgcccaggatccccacgctcaagaatctggaagatctcgtcacagaataccatggtaatttcagcgcctggagcggagtctctaagggtctggccgaatccctccaacccgattattctgaacggttgtgcctcgtatccgaaataccaccaaaaggcggggctctgggtgagggcccaggggcgagtccgtgcaatcaacacagcccgtattgggcccctccttgttatacgttgaagcccgaaactggaagcggagctactaacttcagcctgctgaagcaggctggagacgtggaggagaaccctggacctatggcactgcccgtgaccgccctgctgctgcctctggccctgctgctgcacgcagcccggcctatcctgtggcacgagatgtggcacgagggcctggaggaggccagcaggctgtattttggcgagcgcaacgtgaagggcatgttcgaggtgctggagcctctgcacgccatgatggagagaggcccacagaccctgaaggagacatcctttaaccaggcctatggacgggacctgatggaggcacaggagtggtgcagaaagtacatgaagtctggcaatgtgaaggacctgctgcaggcctgggatctgtactatcacgtgtttcggagaatctccaagccagcagctctcggcaaagacacgattccgtggcttgggcatctgctcgttgggctgagcggtgcgtttggtttcatcatcttggtctatctcttgatcaattgcagaaatacaggcccttggctgaaaaaagtgctcaagtgtaatacccccgacccaagcaagttcttctcccagctttcttcagagcatggaggcgatgtgcagaaatggctctcttcaccttttccctcctcaagcttctccccgggagggctggcgcccgagatttcacctcttgaggtacttgaacgagacaaggttacccaacttctccttcaacaggataaggtacccgaacctgcgagccttagcttgaatacagacgcttatctctcactgcaggaactgcaaggatctggtgctactaatttttctcttttgaagcaagctggagatgttgaagagaaccccggtccggagatgtggcatgagggtctggaagaagcgtctcgactgtactttggtgagcgcaatgtgaagggcatgtttgaagtcctcgaaccccttcatgccatgatggaacgcggaccccagaccttgaaggagacaagttttaaccaagcttacggaagagacctgatggaagcccaggaatggtgcaggaaatacatgaaaagcgggaatgtgaaggacttgctccaagcgtgggacctgtactatcatgtctttaggcgcattagtaag

SEQ ID NO: 45 - DNA, 1632 bp, Artificial Sequence
  CISCbeta-DN nucleotide sequence
atggcactgcccgtgaccgccctgctgctgcctctggccctgctgctgcacgcagcccggcctatcctgtggcacgagatgtggcacgagggcctggaggaggccagcaggctgtattttggcgagcgcaacgtgaagggcatgttcgaggtgctggagcctctgcacgccatgatggagagaggcccacagaccctgaaggagacatcctttaaccaggcctatggacgggacctgatggaggcacaggagtggtgcagaaagtacatgaagtctggcaatgtgaaggacctgctgcaggcctgggatctgtactatcacgtgtttcggagaatctccaagccagcagctctcggcaaagacacgattccgtggcttgggcatctgctcgttgggctgagcggtgcgtttggtttcatcatcttggtctatctcttgatcaattgcagaaatacaggcccttggctgaaaaaagtgctcaagtgtaatacccccgacccaagcaagttcttctcccagctttcttcagagcatggaggcgatgtgcagaaatggctctcttcaccttttccctcctcaagcttctccccgggagggctggcgcccgagatttcacctcttgaggtacttgaacgagacaaggttacccaacttctccttcaacaggataaggtacccgaacctgcgagccttagctccaaccactctcttacgagctgcttcaccaatcagggatacttctttttccaccttcccgatgcgctggaaatcgaagcttgtcaagtttactttacctatgatccatatagcgaggaagatcccgacgaaggagtcgccggtgcgcccacgggttcctcaccccaacctctccagcctctctcaggagaagatgatgcttattgcacttttcccagtagagacgatctcctcctcttttctccatctcttttggggggaccttcccccccttctacggcacctggcgggtctggtgctggcgaggagcggatgccgccgtccctccaggagcgagtaccacgagattgggatccccagccacttggaccccccacccccggcgtacctgaccttgtcgattttcaacctccccctgaattggtgctgcgagaggctggggaggaagttccggacgctgggccgagggagggcgtgtcctttccatggagtaggcctccaggtcaaggcgagtttagggctctcaacgcgcggctgccgttgaatacagacgcttatctctcactgcaggaactgcaaggtcaggacccaacacatcttgtaggatctggtgctactaatttttctcttttgaagcaagctggagatgttgaagagaaccccggtccggagatgtggcatgagggtctggaagaagcgtctcgactgtactttggtgagcgcaatgtgaagggcatgtttgaagtcctcgaaccccttcatgccatgatggaacgcggaccccagaccttgaaggagacaagttttaaccaagcttacggaagagacctgatggaagcccaggaatggtgcaggaaatacatgaaaagcgggaatgtgaaggacttgctccaagcgtgggacctgtactatcatgtctttaggcgcattagtaag

SEQ ID NO: 46 - DNA, 3015 bp, Artificial Sequence
  CISCgamma-FOXP3cDNA-LNGFR nucleotide sequence
atgcctctgggcctgctgtggctgggcctggccctgctgggcgccctgcacgcccaggccggcgtgcaggtggagacaatctccccaggcgacggacgcacattccctaagcggggccagacctgcgtggtgcactatacaggcatgctggaggatggcaagaagtttgacagctcccgggatagaaacaagccattcaagtttatgctgggcaagcaggaagtgatcagaggctgggaggagggcgtggcccagatgtctgtgggccagagggccaagctgaccatcagcccagactacgcctatggagcaacaggccacccaggaatcatcccacctcacgccaccctggtgttcgatgtggagctgctgaagctgggcgagggagggtcacctggatccaacacatcaaaagagaacccctttctgttcgcattggaggccgtagtcatatctgttggatccatgggacttattatctccctgttgtgtgtgtacttctggctggaacggactatgcccaggatccccacgctcaagaatctggaagatctcgtcacagaataccatggtaatttcagcgcctggagcggagtctctaagggtctggccgaatccctccaacccgattattctgaacggttgtgcctcgtatccgaaataccaccaaaaggcggggctctgggtgagggcccaggggcgagtccgtgcaatcaacacagcccgtattgggcccctccttgttatacgttgaagcccgaaactggaagcggagcgactaacttcagcctgcttaagcaggccggagatgtggaggaaaaccctggaccgatgcctaatcctcggcctggaaagcctagcgctccttctcttgctctgggaccttctcctggcgcctctccatcttggagagccgctcctaaagccagcgatctgctgggagctagaggacctggcggcacatttcagggcagagatcttagaggcggagcccacgctagctcctccagccttaatcctatgcctcctagccagctccagctgcctacactgcctctggttatggtggctcctagcggagctagactgggccctctgcctcatctgcaagctctgctgcaggacagaccccacttcatgcaccagctgagcaccgtggatgcccacgcaagaacacctgtgctgcaggttcaccctctggaatccccagccatgatcagcctgacacctccaacaacagccaccggcgtgttcagcctgaaagccagacctggactgcctcctggcatcaatgtggccagcctggaatgggtgtccagagaacctgctctgctgtgcacattccccaatccaagcgctcccagaaaggacagcacactgtctgccgtgcctcagagcagctatcccctgcttgctaacggcgtgtgcaagtggcctggatgcgagaaggtgttcgaggaacccgaggacttcctgaagcactgccaggccgatcatctgctggacgagaaaggcagagcccagtgtctgctccagcgcgagatggtgcagtctctggaacagcagctggtcctggaaaaagaaaagctgagcgccatgcaggcccacctggccggaaaaatggccctgacaaaggccagcagcgtggcctcttctgataagggcagctgctgcattgtggccgctggatctcagggacctgtggttcctgcttggagcggacctagagaggcccctgattctctgtttgccgtgcggagacacctgtggggctctcacggcaactctactttccccgagttcctgcacaacatggactacttcaagttccacaacatgcggcctccattcacctacgccacactgatcagatgggccattctggaagcccctgagaagcagagaaccctgaacgagatctaccactggtttacccggatgttcgccttcttccggaatcaccctgccacctggaagaacgccatccggcacaatctgagcctgcacaagtgcttcgtgcgcgtggaatctgagaaaggcgccgtgtggacagtggacgagctggaattcagaaagaagagaagccagcggcctagccggtgcagcaatcctacacctggacctggaagcggagcgactaacttcagcctgctgaagcaggccggagatgtggaggaaaaccctggaccgatgggggcaggtgccaccggacgagccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggtga

SEQ ID NO: 47 - DNA, 3015 bp, Artificial Sequence
  CISCgamma-LNGFR-FOXP3cDNA nucleotide sequence
atgcctctgggcctgctgtggctgggcctggccctgctgggcgccctgcacgcccaggccggcgtgcaggtggagacaatctccccaggcgacggacgcacattccctaagcggggccagacctgcgtggtgcactatacaggcatgctggaggatggcaagaagtttgacagctcccgggatagaaacaagccattcaagtttatgctgggcaagcaggaagtgatcagaggctgggaggagggcgtggcccagatgtctgtgggccagagggccaagctgaccatcagcccagactacgcctatggagcaacaggccacccaggaatcatcccacctcacgccaccctggtgttcgatgtggagctgctgaagctgggcgagggagggtcacctggatccaacacatcaaaagagaacccctttctgttcgcattggaggccgtagtcatatctgttggatccatgggacttattatctccctgttgtgtgtgtacttctggctggaacggactatgcccaggatccccacgctcaagaatctggaagatctcgtcacagaataccatggtaatttcagcgcctggagcggagtctctaagggtctggccgaatccctccaacccgattattctgaacggttgtgcctcgtatccgaaataccaccaaaaggcggggctctgggtgagggcccaggggcgagtccgtgcaatcaacacagcccgtattgggcccctccttgttatacgttgaagcccgaaactggaagcggagcgactaacttcagcctgcttaagcaggccggagatgtggaggaaaaccctggaccgatgggggcaggtgccaccggacgagccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggggaagcggagcgactaacttcagcctgctgaagcaggccggagatgtggaggaaaaccctggaccgatgcctaatcctcggcctggaaagcctagcgctccttctcttgctctgggaccttctcctggcgcctctccatcttggagagccgctcctaaagccagcgatctgctgggagctagaggacctggcggcacatttcagggcagagatcttagaggcggagcccacgctagctcctccagccttaatcctatgcctcctagccagctccagctgcctacactgcctctggttatggtggctcctagcggagctagactgggccctctgcctcatctgcaagctctgctgcaggacagaccccacttcatgcaccagctgagcaccgtggatgcccacgcaagaacacctgtgctgcaggttcaccctctggaatccccagccatgatcagcctgacacctccaacaacagccaccggcgtgttcagcctgaaagccagacctggactgcctcctggcatcaatgtggccagcctggaatgggtgtccagagaacctgctctgctgtgcacattccccaatccaagcgctcccagaaaggacagcacactgtctgccgtgcctcagagcagctatcccctgcttgctaacggcgtgtgcaagtggcctggatgcgagaaggtgttcgaggaacccgaggacttcctgaagcactgccaggccgatcatctgctggacgagaaaggcagagcccagtgtctgctccagcgcgagatggtgcagtctctggaacagcagctggtcctggaaaaagaaaagctgagcgccatgcaggcccacctggccggaaaaatggccctgacaaaggccagcagcgtggcctcttctgataagggcagctgctgcattgtggccgctggatctcagggacctgtggttcctgcttggagcggacctagagaggcccctgattctctgtttgccgtgcggagacacctgtggggctctcacggcaactctactttccccgagttcctgcacaacatggactacttcaagttccacaacatgcggcctccattcacctacgccacactgatcagatgggccattctggaagcccctgagaagcagagaaccctgaacgagatctaccactggtttacccggatgttcgccttcttccggaatcaccctgccacctggaagaacgccatccggcacaatctgagcctgcacaagtgcttcgtgcgcgtggaatctgagaaaggcgccgtgtggacagtggacgagctggaattcagaaagaagagaagccagcggcctagccggtgcagcaatcctacacctggaccttga

SEQ ID NO: 48 - protein, 251 aa, Artificial Sequence
  IL2Rgamma-CISC
MPLGLLWLGLALLGALHAQAGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLGEGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 49 - protein, 429 aa, Artificial Sequence
  IL2Rbeta-CISC
MALPVTALLLPLALLLHAARPILWHEMWHEGLEEASRLYFGERNVKGMFEVLEPLHAMMERGPQTLKETSFNQAYGRDLMEAQEWCRKYMKSGNVKDLLQAWDLYYHVFRRISKGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 50 - protein, 352 aa, Artificial Sequence
  IL2Rgamma-CISC
MPLGLLWLGLALLGALHAQAGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGGSQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 51 - protein, 543 aa, Artificial Sequence
  IL2Rbeta-CISC
MALPVTALLLPLALLLHAARPILWHEMWHEGLEEASRLYFGERNVKGMFEVLEPLHAMMERGPQTLKETSFNQAYGRDLMEAQEWCRKYMKSGNVKDLLQAWDLYYHVFRRISKGGSKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 52 - protein, 349 aa, Artificial Sequence
  IL2Rgamma-CISC
MPLGLLWLGLALLGALHAQAGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 53 - protein, 541 aa, Artificial Sequence
  IL2Rbeta-CISC
MALPVTALLLPLALLLHAARPILWHEMWHEGLEEASRLYFGERNVKGMFEVLEPLHAMMERGPQTLKETSFNQAYGRDLMEAQEWCRKYMKSGNVKDLLQAWDLYYHVFRRISKKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 54 - protein, 251 aa, Artificial Sequence
  IL2Rgamma-CISC
MPLGLLWLGLALLGALHAQAGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 55 - protein, 379 aa, Artificial Sequence
  IL2Rbeta-CISC
MALPVTALLLPLALLLHAARPILWHEMWHEGLEEASRLYFGERNVKGMFEVLEPLHAMMERGPQTLKETSWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 56 - protein, 345 aa, Artificial Sequence
  IL7Ra-CISC
MALPVTALLLPLALLLHAARPILWHEMWHEGLEEASRLYFGERNVKGMFEVLEPLHAMMERGPQTLKETSFNQAYGRDLMEAQEWCRKYMKSGNVKDLLQAWDLYYHVFRRISKGEINNSSGEMDPILLTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 57 - protein, 443 aa, Artificial Sequence
  IL2Rbeta-CISC
MPLGLLWLGLALLGALHAQAGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEGGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 58 - protein, 251 aa, Artificial Sequence
  IL2Rgamma-CISC
MPLGLLWLGLALLGALHAQAGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEGGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 59 - protein, 413 aa, Artificial Sequence
  IL2Ra-CISC
QRAKLTISPDYAYGATGHPGIIPPHATLVFDVELLKLEGEINNSSGEMDPILLTIMPLGLLWLGLALLGALHAQAGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEGEINNSSGEMDPILLTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 60 - protein, 358 aa, Artificial Sequence
  IL7Ra-CISC
MPLGLLWLGLALLGALHAQAGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEGEINNSSGEMDPILLTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 61 - protein, 276 aa, Artificial Sequence
  MPL-CISC
MPLGLLWLGLALLGALHAQAGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLGEETAWISLVTALHLVLGLSAVLGLLLLRWQFPAHYRRLRHALWPSLPDLHRVLGQYLRDTAALSPPKATVSDTCEEVEPSLLEILPKSSERTPLPLCSSQAQMDYRRLQPSCLGTMPLSVCPPMAESGSCCTTHIANHSYLPLSYWQQP

SEQ ID NO: 62 - DNA, 4 bp, Artificial Sequence
  glycine amino acid spacer
gggs

SEQ ID NO: 63 - DNA, 7 bp, Artificial Sequence
  glycine amino acid spacer
gggsggg

SEQ ID NO: 64 - DNA, 3 bp, Artificial Sequence
  glycine amino acid spacer
ggg

SEQ ID NO: 65 - DNA, 10053 bp, Artificial Sequence
  expression vector
agcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgagaagcttgatatcgaattcccacggggttggacgcgtaggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgctagcaccggtgccgccaccatgcctctgggcctgctgtggctgggcctggccctgctgggcgccctgcacgcccaggccggcgtgcaggtggagacaatctccccaggcgacggacgcacattccctaagcggggccagacctgcgtggtgcactatacaggcatgctggaggatggcaagaagtttgacagctcccgggatagaaacaagccattcaagtttatgctgggcaagcaggaagtgatcagaggctgggaggagggcgtggcccagatgtctgtgggccagagggccaagctgaccatcagcccagactacgcctatggagcaacaggccacccaggaatcatcccacctcacgccaccctggtgttcgatgtggagctgctgaagctgggcgagggcggtagtcagaaccttgtgataccatgggccccagaaaatctcacacttcataaactttccgaatcacaactcgaactcaactggaataaccggttcctgaatcactgtcttgaacacctggtacaatatcggaccgactgggatcactcatggacagaacaatctgtggactataggcacaaattctcactcccaagcgtagacggccaaaaaagatacacttttcgcgtacgatcccgctttaatcctctctgcggctctgctcagcactggagtgaatggtcccatcccattcattggggatccaacacatcaaaagagaacccctttctgttcgcattggaggccgtagtcatatctgttggatccatgggacttattatctccctgttgtgtgtgtacttctggctggaacggactatgcccaggatccccacgctcaagaatctggaagatctcgtcacagaataccatggtaatttcagcgcctggagcggagtctctaagggtctggccgaatccctccaacccgattattctgaacggttgtgcctcgtatccgaaataccaccaaaaggcggggctctgggtgagggcccaggggcgagtccgtgcaatcaacacagcccgtattgggcccctccttgttatacgttgaagcccgaaactggaagcggagctactaacttcagcctgctgaagcaggctggagacgtggaggagaaccctggacctatggcactgcccgtgaccgccctgctgctgcctctggccctgctgctgcacgcagcccggcctatcctgtggcacgagatgtggcacgagggcctggaggaggccagcaggctgtattttggcgagcgcaacgtgaagggcatgttcgaggtgctggagcctctgcacgccatgatggagagaggcccacagaccctgaaggagacatcctttaaccaggcctatggacgggacctgatggaggcacaggagtggtgcagaaagtacatgaagtctggcaatgtgaaggacctgctgcaggcctgggatctgtactatcacgtgtttcggagaatctccaagggaggttcaaaaccttttgagaaccttagactgatggcgcccatctctctgcaggtagttcacgttgagacccatagatgcaatataagctgggaaatctcacaagccagccattactttgaacggcatttggaattcgaggcccgaacactttcccccggtcatacgtgggaagaagctcctctcttgacgctgaagcagaagcaggagtggatttgtctggagactttgactcctgatactcagtatgagttccaagttcgggtgaaaccactccaaggcgagttcacgacgtggtctccgtggagtcaaccgttggcgttccgcacgaagcccgctgcccttggcaaagacacgattccgtggcttgggcatctgctcgttgggctgagtggtgcgtttggtttcatcatcttggtctatctcttgatcaattgcagaaatacaggcccttggctgaaaaaagtgctcaagtgtaatacccccgacccaagcaagttcttctcccagctttcttcagagcatggaggcgatgtgcagaaatggctctcttcaccttttccctcctcaagcttctccccgggagggctggcgcccgagatttcacctcttgaggtacttgaacgagacaaggttacccaacttctccttcaacaggataaggtacccgaacctgcgagccttagctccaaccactctcttacgagctgcttcaccaatcagggatacttctttttccaccttcccgatgcgctggaaatcgaagcttgtcaagtttactttacctatgatccatatagcgaggaagatcccgacgaaggagtcgccggtgcgcccacgggttcctcaccccaacctctccagcctctctcaggagaagatgatgcttattgcacttttcccagtagagacgatctcctcctcttttctccatctcttttggggggaccttcccccccttctacggcacctggcgggtctggtgctggcgaggagcggatgccgccgtccctccaggagcgagtaccacgagattgggatccccagccacttggaccccccacccccggcgtacctgaccttgtcgattttcaacctccccctgaattggtgctgcgagaggctggggaggaagttccggacgctgggccgagggagggcgtgtcctttccatggagtaggcctccaggtcaaggcgagtttagggctctcaacgcgcggctgccgttgaatacagacgcttatctctcactgcaggaactgcaaggtcaggacccaacacatcttgtaggatctggtgctactaatttttctcttttgaagcaagctggagatgttgaagagaaccctggtccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaactagtgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgca

SEQ ID NO: 66 - DNA, 10035 bp, Artificial Sequence
  expression vector
agcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgagaagcttgatatcgaattcccacggggttggacgcgtaggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgctagcaccggtgccgccaccatgcctctgggcctgctgtggctgggcctggccctgctgggcgccctgcacgcccaggccggcgtgcaggtggagacaatctccccaggcgacggacgcacattccctaagcggggccagacctgcgtggtgcactatacaggcatgctggaggatggcaagaagtttgacagctcccgggatagaaacaagccattcaagtttatgctgggcaagcaggaagtgatcagaggctgggaggagggcgtggcccagatgtctgtgggccagagggccaagctgaccatcagcccagactacgcctatggagcaacaggccacccaggaatcatcccacctcacgccaccctggtgttcgatgtggagctgctgaagctgggcgagcaaaacttggtgattccttgggccccagaaaatctcacgcttcacaagttgtccgaatcccagctcgagctcaactggaataatagatttcttaatcattgtttggaacacctggttcaatatagaacggattgggaccactcatggaccgagcagtcagttgactaccgccacaaattttcacttcccagcgtagatgggcagaagaggtacacatttagggtcagatccaggtttaatcctctgtgtggttctgctcaacactggtctgagtggagccatccgatccactggggctcaaatacctctaaagaaaatccgttcctctttgcgctcgaagccgttgttatcagcgtcggaagcatgggacttatcatttcccttctctgcgtgtacttctggctggagcggacgatgccgcggattccgacgctcaaaaacctggaggaccttgtaacagaatatcacggtaatttctccgcttggagtggcgtatcaaaggggcttgctgagtcccttcaaccggattactctgagcgcctctgcttggtgtccgagatacctcccaaaggaggtgcacttggggaggggccaggcgcgtccccttgcaatcagcatagtccgtattgggcgcccccctgttataccctcaaaccggaaacgggaagcggagctactaacttcagcctgctgaagcaggctggagacgtggaggagaaccctggacctatggcactgcccgtgaccgccctgctgctgcctctggccctgctgctgcacgcagcccggcctatcctgtggcacgagatgtggcacgagggcctggaggaggccagcaggctgtattttggcgagcgcaacgtgaagggcatgttcgaggtgctggagcctctgcacgccatgatggagagaggcccacagaccctgaaggagacatcctttaaccaggcctatggacgggacctgatggaggcacaggagtggtgcagaaagtacatgaagtctggcaatgtgaaggacctgctgcaggcctgggatctgtactatcacgtgtttcggagaatctccaagaaaccttttgagaaccttagactgatggcgcccatctctctgcaggtagttcacgttgagacccatagatgcaatataagctgggaaatctcacaagccagccattactttgaacggcatttggaattcgaggcccgaacactttcccccggtcatacgtgggaagaagctcctctcttgacgctgaagcagaagcaggagtggatttgtctggagactttgactcctgatactcagtatgagttccaagttcgggtgaaaccactccaaggcgagttcacgacgtggtctccgtggagtcaaccgttggcgttccgcacgaagcccgctgcccttggcaaagacacgattccgtggcttgggcatctgctcgttgggctgagtggtgcgtttggtttcatcatcttggtctatctcttgatcaattgcagaaatacaggcccttggctgaaaaaagtgctcaagtgtaatacccccgacccaagcaagttcttctcccagctttcttcagagcatggaggcgatgtgcagaaatggctctcttcaccttttccctcctcaagcttctccccgggagggctggcgcccgagatttcacctcttgaggtacttgaacgagacaaggttacccaacttctccttcaacaggataaggtacccgaacctgcgagccttagctccaaccactctcttacgagctgcttcaccaatcagggatacttctttttccaccttcccgatgcgctggaaatcgaagcttgtcaagtttactttacctatgatccatatagcgaggaagatcccgacgaaggagtcgccggtgcgcccacgggttcctcaccccaacctctccagcctctctcaggagaagatgatgcttattgcacttttcccagtagagacgatctcctcctcttttctccatctcttttggggggaccttcccccccttctacggcacctggcgggtctggtgctggcgaggagcggatgccgccgtccctccaggagcgagtaccacgagattgggatccccagccacttggaccccccacccccggcgtacctgaccttgtcgattttcaacctccccctgaattggtgctgcgagaggctggggaggaagttccggacgctgggccgagggagggcgtgtcctttccatggagtaggcctccaggtcaaggcgagtttagggctctcaacgcgcggctgccgttgaatacagacgcttatctctcactgcaggaactgcaaggtcaggacccaacacatcttgtaggatctggtgctactaatttttctcttttgaagcaagctggagatgttgaagagaaccctggtccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaactagtgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgca

SEQ ID NO: 67 - DNA, 9405 bp, Artificial Sequence
  expression vector
agcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgagaagcttgatatcgaattcccacggggttggacgcgtaggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgctagcaccggtgccgccaccatgcctctgggcctgctgtggctgggcctggccctgctgggcgccctgcacgcccaggccggcgtgcaggtggagacaatctccccaggcgacggacgcacattccctaagcggggccagacctgcgtggtgcactatacaggcatgctggaggatggcaagaagtttgacagctcccgggatagaaacaagccattcaagtttatgctgggcaagcaggaagtgatcagaggctgggaggagggcgtggcccagatgtctgtgggccagagggccaagctgaccatcagcccagactacgcctatggagcaacaggccacccaggaatcatcccacctcacgccaccctggtgttcgatgtggagctgctgaagctgggcgagggatccaacacatcaaaagagaacccctttctgttcgcattggaggccgtagtcatatctgttggatccatgggacttattatctccctgttgtgtgtgtacttctggctggaacggactatgcccaggatccccacgctcaagaatctggaagatctcgtcacagaataccatggtaatttcagcgcctggagcggagtctctaagggtctggccgaatccctccaacccgattattctgaacggttgtgcctcgtatccgaaataccaccaaaaggcggggctctgggtgagggcccaggggcgagtccgtgcaatcaacacagcccgtattgggcccctccttgttatacgttgaagcccgaaactggaagcggagctactaacttcagcctgctgaagcaggctggagacgtggaggagaaccctggacctatggcactgcccgtgaccgccctgctgctgcctctggccctgctgctgcacgcagcccggcctatcctgtggcacgagatgtggcacgagggcctggaggaggccagcaggctgtattttggcgagcgcaacgtgaagggcatgttcgaggtgctggagcctctgcacgccatgatggagagaggcccacagaccctgaaggagacatcctttaaccaggcctatggacgggacctgatggaggcacaggagtggtgcagaaagtacatgaagtctggcaatgtgaaggacctgctgcaggcctgggatctgtactatcacgtgtttcggagaatctccaagggcaaagacacgattccgtggcttgggcatctgctcgttgggctgagtggtgcgtttggtttcatcatcttggtctatctcttgatcaattgcagaaatacaggcccttggctgaaaaaagtgctcaagtgtaatacccccgacccaagcaagttcttctcccagctttcttcagagcatggaggcgatgtgcagaaatggctctcttcaccttttccctcctcaagcttctccccgggagggctggcgcccgagatttcacctcttgaggtacttgaacgagacaaggttacccaacttctccttcaacaggataaggtacccgaacctgcgagccttagctccaaccactctcttacgagctgcttcaccaatcagggatacttctttttccaccttcccgatgcgctggaaatcgaagcttgtcaagtttactttacctatgatccatatagcgaggaagatcccgacgaaggagtcgccggtgcgcccacgggttcctcaccccaacctctccagcctctctcaggagaagatgatgcttattgcacttttcccagtagagacgatctcctcctcttttctccatctcttttggggggaccttcccccccttctacggcacctggcgggtctggtgctggcgaggagcggatgccgccgtccctccaggagcgagtaccacgagattgggatccccagccacttggaccccccacccccggcgtacctgaccttgtcgattttcaacctccccctgaattggtgctgcgagaggctggggaggaagttccggacgctgggccgagggagggcgtgtcctttccatggagtaggcctccaggtcaaggcgagtttagggctctcaacgcgcggctgccgttgaatacagacgcttatctctcactgcaggaactgcaaggtcaggacccaacacatcttgtaggatctggtgctactaatttttctcttttgaagcaagctggagatgttgaagagaaccctggtccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaactagtgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgca

SEQ ID NO: 68 - DNA, 1293 bp, Artificial Sequence
  Codon-optimized human FOXP3 cDNA, Without stop codon
atgcctaatcctcggcctggaaagcctagcgctccttctcttgctctgggaccttctcctggcgcctctccatcttggagagccgctcctaaagccagcgatctgctgggagctagaggacctggcggcacatttcagggcagagatcttagaggcggagcccacgctagctcctccagccttaatcctatgcctcctagccagctccagctgcctacactgcctctggttatggtggctcctagcggagctagactgggccctctgcctcatctgcaagctctgctgcaggacagaccccacttcatgcaccagctgagcaccgtggatgcccacgcaagaacacctgtgctgcaggttcaccctctggaatccccagccatgatcagcctgacacctccaacaacagccaccggcgtgttcagcctgaaagccagacctggactgcctcctggcatcaatgtggccagcctggaatgggtgtccagagaacctgctctgctgtgcacattccccaatccaagcgctcccagaaaggacagcacactgtctgccgtgcctcagagcagctatcccctgcttgctaacggcgtgtgcaagtggcctggatgcgagaaggtgttcgaggaacccgaggacttcctgaagcactgccaggccgatcatctgctggacgagaaaggcagagcccagtgtctgctccagcgcgagatggtgcagtctctggaacagcagctggtcctggaaaaagaaaagctgagcgccatgcaggcccacctggccggaaaaatggccctgacaaaggccagcagcgtggcctcttctgataagggcagctgctgcattgtggccgctggatctcagggacctgtggttcctgcttggagcggacctagagaggcccctgattctctgtttgccgtgcggagacacctgtggggctctcacggcaactctactttccccgagttcctgcacaacatggactacttcaagttccacaacatgcggcctccattcacctacgccacactgatcagatgggccattctggaagcccctgagaagcagagaaccctgaacgagatctaccactggtttacccggatgttcgccttcttccggaatcaccctgccacctggaagaacgccatccggcacaatctgagcctgcacaagtgcttcgtgcgcgtggaatctgagaaaggcgccgtgtggacagtggacgagctggaattcagaaagaagagaagccagcggcctagccggtgcagcaatcctacacctggacct

SEQ ID NO: 69 - DNA, 1296 bp, Artificial Sequence
  Codon-optimized human FOXP3 cDNA, With stop codon
atgcctaatcctcggcctggaaagcctagcgctccttctcttgctctgggaccttctcctggcgcctctccatcttggagagccgctcctaaagccagcgatctgctgggagctagaggacctggcggcacatttcagggcagagatcttagaggcggagcccacgctagctcctccagccttaatcctatgcctcctagccagctccagctgcctacactgcctctggttatggtggctcctagcggagctagactgggccctctgcctcatctgcaagctctgctgcaggacagaccccacttcatgcaccagctgagcaccgtggatgcccacgcaagaacacctgtgctgcaggttcaccctctggaatccccagccatgatcagcctgacacctccaacaacagccaccggcgtgttcagcctgaaagccagacctggactgcctcctggcatcaatgtggccagcctggaatgggtgtccagagaacctgctctgctgtgcacattccccaatccaagcgctcccagaaaggacagcacactgtctgccgtgcctcagagcagctatcccctgcttgctaacggcgtgtgcaagtggcctggatgcgagaaggtgttcgaggaacccgaggacttcctgaagcactgccaggccgatcatctgctggacgagaaaggcagagcccagtgtctgctccagcgcgagatggtgcagtctctggaacagcagctggtcctggaaaaagaaaagctgagcgccatgcaggcccacctggccggaaaaatggccctgacaaaggccagcagcgtggcctcttctgataagggcagctgctgcattgtggccgctggatctcagggacctgtggttcctgcttggagcggacctagagaggcccctgattctctgtttgccgtgcggagacacctgtggggctctcacggcaactctactttccccgagttcctgcacaacatggactacttcaagttccacaacatgcggcctccattcacctacgccacactgatcagatgggccattctggaagcccctgagaagcagagaaccctgaacgagatctaccactggtttacccggatgttcgccttcttccggaatcaccctgccacctggaagaacgccatccggcacaatctgagcctgcacaagtgcttcgtgcgcgtggaatctgagaaaggcgccgtgtggacagtggacgagctggaattcagaaagaagagaagccagcggcctagccggtgcagcaatcctacacctggaccttga

SEQ ID NO: 70 - protein, 90 aa, Artificial Sequence
  naked FRB domaincodon
MEMWHEGLEEASRLYFGERNVKGMFEVLEPLHAMMERGPQTLKETSFNQAYGRDLMEAQEWCRKYMKSGNVKDLTQAWDLYYHVFRRISK

SEQ ID NO: 71 - protein, 90 aa, Artificial Sequence
  mutant naked FRB domain
MEMWHEGLEEASRLYFGERNVKGMFEVLEPLHAMMERGPQTLKETSFNQAYGRDLMEAQEWCRKYMKSGNVKDLLQAWDLYYHVFRRISK

SEQ ID NO: 72 - DNA, 3805 bp, Artificial Sequence
  MND-FOXP3cDNA-microDISC-SV40 polyA
gaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggatctcgaggccaccatgcctaatcctcggcctggaaagcctagcgctccttctcttgctctgggaccttctcctggcgcctctccatcttggagagccgctcctaaagccagcgatctgctgggagctagaggacctggcggcacatttcagggcagagatcttagaggcggagcccacgctagctcctccagccttaatcctatgcctcctagccagctccagctgcctacactgcctctggttatggtggctcctagcggagctagactgggccctctgcctcatctgcaagctctgctgcaggacagaccccacttcatgcaccagctgagcaccgtggatgcccacgcaagaacacctgtgctgcaggttcaccctctggaatccccagccatgatcagcctgacacctccaacaacagccaccggcgtgttcagcctgaaagccagacctggactgcctcctggcatcaatgtggccagcctggaatgggtgtccagagaacctgctctgctgtgcacattccccaatccaagcgctcccagaaaggacagcacactgtctgccgtgcctcagagcagctatcccctgcttgctaacggcgtgtgcaagtggcctggatgcgagaaggtgttcgaggaacccgaggacttcctgaagcactgccaggccgatcatctgctggacgagaaaggcagagcccagtgtctgctccagcgcgagatggtgcagtctctggaacagcagctggtcctggaaaaagaaaagctgagcgccatgcaggcccacctggccggaaaaatggccctgacaaaggccagcagcgtggcctcttctgataagggcagctgctgcattgtggccgctggatctcagggacctgtggttcctgcttggagcggacctagagaggcccctgattctctgtttgccgtgcggagacacctgtggggctctcacggcaactctactttccccgagttcctgcacaacatggactacttcaagttccacaacatgcggcctccattcacctacgccacactgatcagatgggccattctggaagcccctgagaagcagagaaccctgaacgagatctaccactggtttacccggatgttcgccttcttccggaatcaccctgccacctggaagaacgccatccggcacaatctgagcctgcacaagtgcttcgtgcgcgtggaatctgagaaaggcgccgtgtggacagtggacgagctggaattcagaaagaagagaagccagcggcctagccggtgcagcaatcctacacctggacctggaagcggagcgactaacttcagcctgcttaagcaggccggagatgtggaggaaaaccctggaccgatgcctctgggcctgctgtggctgggcctggccctgctgggcgccctgcacgcccaggccggcgtgcaggtggagacaatctccccaggcgacggacgcacattccctaagcggggccagacctgcgtggtgcactatacaggcatgctggaggatggcaagaagtttgacagctcccgggatagaaacaagccattcaagtttatgctgggcaagcaggaagtgatcagaggctgggaggagggcgtggcccagatgtctgtgggccagagggccaagctgaccatcagcccagactacgcctatggagcaacaggccacccaggaatcatcccacctcacgccaccctggtgttcgatgtggagctgctgaagctgggcgagggagggtcacctggatccaacacatcaaaagagaacccctttctgttcgcattggaggccgtagtcatatctgttggatccatgggacttattatctccctgttgtgtgtgtacttctggctggaacggactatgcccaggatccccacgctcaagaatctggaagatctcgtcacagaataccatggtaatttcagcgcctggagcggagtctctaagggtctggccgaatccctccaacccgattattctgaacggttgtgcctcgtatccgaaataccaccaaaaggcggggctctgggtgagggcccaggggcgagtccgtgcaatcaacacagcccgtattgggcccctccttgttatacgttgaagcccgaaactggaagcggagctactaacttcagcctgctgaagcaggctggagacgtggaggagaaccctggacctatggcactgcccgtgaccgccctgctgctgcctctggccctgctgctgcacgcagcccggcctatcctgtggcacgagatgtggcacgagggcctggaggaggccagcaggctgtattttggcgagcgcaacgtgaagggcatgttcgaggtgctggagcctctgcacgccatgatggagagaggcccacagaccctgaaggagacatcctttaaccaggcctatggacgggacctgatggaggcacaggagtggtgcagaaagtacatgaagtctggcaatgtgaaggacctgctgcaggcctgggatctgtactatcacgtgtttcggagaatctccaagccagcagctctcggcaaagacacgattccgtggcttgggcatctgctcgttgggctgagcggtgcgtttggtttcatcatcttggtctatctcttgatcaattgcagaaatacaggcccttggctgaaaaaagtgctcaagtgtaatacccccgacccaagcaagttcttctcccagctttcttcagagcatggaggcgatgtgcagaaatggctctcttcaccttttccctcctcaagcttctccccgggagggctggcgcccgagatttcacctcttgaggtacttgaacgagacaaggttacccaacttctccttcaacaggataaggtacccgaacctgcgagccttagcttgaatacagacgcttatctctcactgcaggaactgcaaggatctggtgctactaatttttctcttttgaagcaagctggagatgttgaagagaaccccggtccggagatgtggcatgagggtctggaagaagcgtctcgactgtactttggtgagcgcaatgtgaagggcatgtttgaagtcctcgaaccccttcatgccatgatggaacgcggaccccagaccttgaaggagacaagttttaaccaagcttacggaagagacctgatggaagcccaggaatggtgcaggaaatacatgaaaagcgggaatgtgaaggacttgctccaagcgtgggacctgtactatcatgtctttaggcgcattagtaagtgagtcgactgctttatttgtgaaatttgtgatgctattgctttatttgtaaccattataagctgcaataaacaagttaacaacaacaattgcattcattttatgtttcaggttcagggggagatgtgggaggttttttaaagc

SEQ ID NO: 73 - protein, 1086 aa, Artificial Sequence
  FOXP3cDNA-microDISC amino acid sequence
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGPGSGATNFSLLKQAGDVEENPGPMPLGLLWLGLALLGALHAQAGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLGEGGSPGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPETGSGATNFSLLKQAGDVEENPGPMALPVTALLLPLALLLHAARPILWHEMWHEGLEEASRLYFGERNVKGMFEVLEPLHAMMERGPQTLKETSFNQAYGRDLMEAQEWCRKYMKSGNVKDLLQAWDLYYHVFRRISKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSLNTDAYLSLQELQGSGATNFSLLKQAGDVEENPGPEMWHEGLEEASRLYFGERNVKGMFEVLEPLHAMMERGPQTLKETSFNQAYGRDLMEAQEWCRKYMKSGNVKDLLQAWDLYYHVFRRISK

SEQ ID NO: 74 - protein, 1360 aa, Artificial Sequence
  FOXP3cDNA-LNGFRe-microDISC amino acid sequence
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGPGSGATNFSLLKQAGDVEENPGPMPLGLLWLGLALLGALHAQAMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLGEGGSPGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPETGSGATNFSLLKQAGDVEENPGPMALPVTALLLPLALLLHAARPILWHEMWHEGLEEASRLYFGERNVKGMFEVLEPLHAMMERGPQTLKETSFNQAYGRDLMEAQEWCRKYMKSGNVKDLLQAWDLYYHVFRRISKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSLNTDAYLSLQELQGSGATNFSLLKQAGDVEENPGPEMWHEGLEEASRLYFGERNVKGMFEVLEPLHAMMERGPQTLKETSFNQAYGRDLMEAQEWCRKYMKSGNVKDLLQAWDLYYHVFRRISK

SEQ ID NO: 75 - protein, 1086 aa, Artificial Sequence
  microDISC-FOXP3cDNA amino acid sequence
MPLGLLWLGLALLGALHAQAGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLGEGGSPGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPETGSGATNFSLLKQAGDVEENPGPMALPVTALLLPLALLLHAARPILWHEMWHEGLEEASRLYFGERNVKGMFEVLEPLHAMMERGPQTLKETSFNQAYGRDLMEAQEWCRKYMKSGNVKDLLQAWDLYYHVFRRISKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSLNTDAYLSLQELQGSGATNFSLLKQAGDVEENPGPEMWHEGLEEASRLYFGERNVKGMFEVLEPLHAMMERGPQTLKETSFNQAYGRDLMEAQEWCRKYMKSGNVKDLLQAWDLYYHVFRRISKGSGATNFSLLKQAGDVEENPGPMPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGP

SEQ ID NO: 76 - protein, 1360 aa, Artificial Sequence
  LNGFRe-microDISC -FOXP3cDNA amino acid sequence
MPLGLLWLGLALLGALHAQAMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLGEGGSPGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPETGSGATNFSLLKQAGDVEENPGPMALPVTALLLPLALLLHAARPILWHEMWHEGLEEASRLYFGERNVKGMFEVLEPLHAMMERGPQTLKETSFNQAYGRDLMEAQEWCRKYMKSGNVKDLLQAWDLYYHVFRRISKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSLNTDAYLSLQELQGSGATNFSLLKQAGDVEENPGPEMWHEGLEEASRLYFGERNVKGMFEVLEPLHAMMERGPQTLKETSFNQAYGRDLMEAQEWCRKYMKSGNVKDLLQAWDLYYHVFRRISKGSGATNFSLLKQAGDVEENPGPMPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGP

SEQ ID NO: 77 - protein, 821 aa, Artificial Sequence
  DISC amino acid sequence
MPLGLLWLGLALLGALHAQAGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLGEGGSPGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPETGSGATNFSLLKQAGDVEENPGPMALPVTALLLPLALLLHAARPILWHEMWHEGLEEASRLYFGERNVKGMFEVLEPLHAMMERGPQTLKETSFNQAYGRDLMEAQEWCRKYMKSGNVKDLLQAWDLYYHVFRRISKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLVGSGATNFSLLKQAGDVEENPGPEMWHEGLEEASRLYFGERNVKGMFEVLEPLHAMMERGPQTLKETSFNQAYGRDLMEAQEWCRKYMKSGNVKDLLQAWDLYYHVFRRISK

SEQ ID NO: 78 - protein, 633 aa, Artificial Sequence
  microDISC amino acid sequence
MPLGLLWLGLALLGALHAQAGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLGEGGSPGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPETGSGATNFSLLKQAGDVEENPGPMALPVTALLLPLALLLHAARPILWHEMWHEGLEEASRLYFGERNVKGMFEVLEPLHAMMERGPQTLKETSFNQAYGRDLMEAQEWCRKYMKSGNVKDLLQAWDLYYHVFRRISKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSLNTDAYLSLQELQGSGATNFSLLKQAGDVEENPGPEMWHEGLEEASRLYFGERNVKGMFEVLEPLHAMMERGPQTLKETSFNQAYGRDLMEAQEWCRKYMKSGNVKDLLQAWDLYYHVFRRISK

SEQ ID NO: 79 - protein, 544 aa, Artificial Sequence
  CISCbeta-DN amino acid sequence
MALPVTALLLPLALLLHAARPILWHEMWHEGLEEASRLYFGERNVKGMFEVLEPLHAMMERGPQTLKETSFNQAYGRDLMEAQEWCRKYMKSGNVKDLLQAWDLYYHVFRRISKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLVGSGATNFSLLKQAGDVEENPGPEMWHEGLEEASRLYFGERNVKGMFEVLEPLHAMMERGPQTLKETSFNQAYGRDLMEAQEWCRKYMKSGNVKDLLQAWDLYYHVFRRISK

SEQ ID NO: 80 - protein, 1004 aa, Artificial Sequence
  CISCgamma-FOXP3cDNA-LNGFR amino acid sequence
MPLGLLWLGLALLGALHAQAGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLGEGGSPGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPETGSGATNFSLLKQAGDVEENPGPMPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGPGSGATNFSLLKQAGDVEENPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKR

SEQ ID NO: 81 - DNA, 3015 bp, Artificial Sequence; identical to SEQ ID NO: 47
  CISCgamma-LNGFR-FOXP3cDNA

SEQ ID NO: 82 - DNA, 765 bp, Artificial Sequence
  CISCgamma: FKBP-IL2Rgamma; nucleotide sequence
atgcctctgggcctgctgtggctgggcctggccctgctgggcgccctgcacgcccaggccggcgtgcaggtggagacaatctccccaggcgacggacgcacattccctaagcggggccagacctgcgtggtgcactatacaggcatgctggaggatggcaagaagtttgacagctcccgggatagaaacaagccattcaagtttatgctgggcaagcaggaagtgatcagaggctgggaggagggcgtggcccagatgtctgtgggccagagggccaagctgaccatcagcccagactacgcctatggagcaacaggccacccaggaatcatcccacctcacgccaccctggtgttcgatgtggagctgctgaagctgggcgagggagggtcacctggatccaacacatcaaaagagaacccctttctgttcgcattggaggccgtagtcatatctgttggatccatgggacttattatctccctgttgtgtgtgtacttctggctggaacggactatgcccaggatccccacgctcaagaatctggaagatctcgtcacagaataccatggtaatttcagcgcctggagcggagtctctaagggtctggccgaatccctccaacccgattattctgaacggttgtgcctcgtatccgaaataccaccaaaaggcggggctctgggtgagggcccaggggcgagtccgtgcaatcaacacagcccgtattgggcccctccttgttatacgttgaagcccgaaact

SEQ ID NO: 83 - protein, 255 aa, Artificial Sequence
  CISCgamma: FKBP-IL2Rgamma amino acid sequence
MPLGLLWLGLALLGALHAQAGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLGEGGSPGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 84 - skipped

SEQ ID NO: 85 - protein, 821 aa, Artificial Sequence; identical to SEQ ID NO: 77
  DISC: CISC-FRB; microDISC amino acid sequence

SEQ ID NO: 86 - DNA, 267 bp, Artificial Sequence
  FRB: express intracellularly to function as a decoy for rapamycin: FRB; nucleotide sequence
gagatgtggcatgagggtctggaagaagcgtctcgactgtactttggtgagcgcaatgtgaagggcatgtttgaagtcctcgaaccccttcatgccatgatggaacgcggaccccagaccttgaaggagacaagttttaaccaagcttacggaagagacctgatggaagcccaggaatggtgcaggaaatacatgaaaagcgggaatgtgaaggacttgacccaagcgtgggacctgtactatcatgtctttaggcgcattagtaag

SEQ ID NO: 87 - protein, 89 aa, Artificial Sequence
  FRB amino acid sequence
EMWHEGLEEASRLYFGERNVKGMFEVLEPLHAMMERGPQTLKETSFNQAYGRDLMEAQEWCRKYMKSGNVKDLTQAWDLYYHVFRRISK

SEQ ID NO: 88 - DNA, 825 bp, Artificial Sequence
  LNGFR coding sequence with stop codon
atgggggcaggtgccaccggacgagccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggtga

SEQ ID NO: 89 - DNA, 66 bp, Artificial Sequence
  P2A self-cleaving peptide
ggaagcggagcgactaacttcagcctgctgaagcaggccggagatgtggaggaaaaccctggaccg

SEQ ID NO: 90 - DNA, 258 bp, Artificial Sequence
  0.25kb human FOXP3 5prime HA designed for both TALEN and Cas9 approaches
tgctagcgtgggcaggcaagccaggtgctggacctctgcacgtggggcatgtgtgggtatgtacatgtacctgtgttcttggtgtgtgtgtgtgtgtgtgtgtgtgtgtgtgtctagagctggggtgcaactatggggcccctcgggacatgtcccagccaatgcctgctttgaccagaggagtgtccacgtggctcaggtggtcgagtatctcataccgccctagcacacgtgtgactcctttcccctattgtctac

SEQ ID NO: 91 - DNA, 296 bp, Artificial Sequence
  0.3kb human FOXP3 5 primer HA for Cas9-T9
catgtgtgggtatgtacatgtacctgtgttcttggtgtgtgtgtgtgtgtgtgtgtgtgtgtgtgtctagagctggggtgcaactatggggcccctcgggacatgtcccagccaatgcctgctttgaccagaggagtgtccacgtggctcaggtggtcgagtatctcataccgccctagcacacgtgtgactcctttcccctattgtctacgcagcctgcccttggacaaggacccgatgcccaaccccaggcctggcaagccctcggccccttccttggcccttggcccatcccc

SEQ ID NO: 92 - DNA, 452 bp, Artificial Sequence
  0.45kb human FOXP3 5 primer HA for Cas9-T9
agcctgtgcagggtgcagggagggctagaggcctgaggcttgaaacagctctcaagtggagggggaaacaaccattgccctcatagaggacacatccacaccagggctgtgctagcgtgggcaggcaagccaggtgctggacctctgcacgtggggcatgtgtgggtatgtacatgtacctgtgttcttggtgtgtgtgtgtgtgtgtgtgtgtgtgtgtgtctagagctggggtgcaactatggggcccctcgggacatgtcccagccaatgcctgctttgaccagaggagtgtccacgtggctcaggtggtcgagtatctcataccgccctagcacacgtgtgactcctttcccctattgtctacgcagcctgcccttggacaaggacccgatgcccaaccccaggcctggcaagccctcggccccttccttggcccttggcccatcccc

SEQ ID NO: 93 - DNA, 600 bp, Artificial Sequence
  0.6kb human FOXP3 5 primer HA for Cas9-T9
atcacttgccaggactgttacaatagcctcctcactagccccactcacagcagccagatgaatcttttgagtccatgcctagtcactggggcaaaataggactccgaggagaaagtccgagaccagctccggcaagatgagcaaacacagcctgtgcagggtgcagggagggctagaggcctgaggcttgaaacagctctcaagtggagggggaaacaaccattgccctcatagaggacacatccacaccagggctgtgctagcgtgggcaggcaagccaggtgctggacctctgcacgtggggcatgtgtgggtatgtacatgtacctgtgttcttggtgtgtgtgtgtgtgtgtgtgtgtgtgtgtgtctagagctggggtgcaactatggggcccctcgggacatgtcccagccaatgcctgctttgaccagaggagtgtccacgtggctcaggtggtcgagtatctcataccgccctagcacacgtgtgactcctttcccctattgtctacgcagcctgcccttggacaaggacccgatgcccaaccccaggcctggcaagccctcggccccttccttggcccttggcccatcccc

SEQ ID NO: 94 - DNA, 785 bp, Artificial Sequence
  0.8kb human FOXP3 5 primer HA for Cas9-T9
atctcaggtaatgtcagctcggtccttccagctgctcaagctaaaacccatgtcactttgactctccctcttgcccactacatccaagctgctagcactgctcctgatccagcttcagattaagtctcagaatctacccacttctcgccttctccactgccaccagcccattctgtgccagcatcatcacttgccaggactgttacaatagcctcctcactagccccactcacagcagccagatgaatcttttgagtccatgcctagtcactggggcaaaataggactccgaggagaaagtccgagaccagctccggcaagatgagcaaacacagcctgtgcagggtgcagggagggctagaggcctgaggcttgaaacagctctcaagtggagggggaaacaaccattgccctcatagaggacacatccacaccagggctgtgctagcgtgggcaggcaagccaggtgctggacctctgcacgtggggcatgtgtgggtatgtacatgtacctgtgttcttggtgtgtgtgtgtgtgtgtgtgtgtgtgtgtgtctagagctggggtgcaactatggggcccctcgggacatgtcccagccaatgcctgctttgaccagaggagtgtccacgtggctcaggtggtcgagtatctcataccgccctagcacacgtgtgactcctttcccctattgtctacgcagcctgcccttggacaaggacccgatgcccaaccccaggcctggcaagccctcggccccttccttggcccttggcccatcccc

SEQ ID NO: 95 - DNA, 275 bp, Artificial Sequence
  0.3kb human FOXP3 5 primer HA for Cas9-T3 (actual length 0.275kb)
gacatgtcccagccaatgcctgctttgaccagaggagtgtccacgtggctcaggtggtcgagtatctcataccgccctagcacacgtgtgactcctttcccctattgtctacgcagcctgcccttggacaaggacccgatgcccaaccccaggcctggcaagccctcggccccttccttggcccttggcccatccccaggagcctcgcccagctggagggctgcacccaaagcctcagacctgctgggggcccggggcccagggggaaccttcca

SEQ ID NO: 96 - DNA, 449 bp, Artificial Sequence
  0.45kb human FOXP3 5 primer HA for Cas9-T3
catagaggacacatccacaccagggctgtgctagcgtgggcaggcaagccaggtgctggacctctgcacgtggggcatgtgtgggtatgtacatgtacctgtgttcttggtgtgtgtgtgtgtgtgtgtgtgtgtgtgtgtctagagctggggtgcaactatggggcccctcgggacatgtcccagccaatgcctgctttgaccagaggagtgtccacgtggctcaggtggtcgagtatctcataccgccctagcacacgtgtgactcctttcccctattgtctacgcagcctgcccttggacaaggacccgatgcccaaccccaggcctggcaagccctcggccccttccttggcccttggcccatccccaggagcctcgcccagctggagggctgcacccaaagcctcagacctgctgggggcccggggcccagggggaaccttcca

SEQ ID NO: 97 - DNA, 600 bp, Artificial Sequence
  0.6kb human FOXP3 5 primer HA for Cas9-T3
ctagtcactggggcaaaataggactccgaggagaaagtccgagaccagctccggcaagatgagcaaacacagcctgtgcagggtgcagggagggctagaggcctgaggcttgaaacagctctcaagtggagggggaaacaaccattgccctcatagaggacacatccacaccagggctgtgctagcgtgggcaggcaagccaggtgctggacctctgcacgtggggcatgtgtgggtatgtacatgtacctgtgttcttggtgtgtgtgtgtgtgtgtgtgtgtgtgtgtgtctagagctggggtgcaactatggggcccctcgggacatgtcccagccaatgcctgctttgaccagaggagtgtccacgtggctcaggtggtcgagtatctcataccgccctagcacacgtgtgactcctttcccctattgtctacgcagcctgcccttggacaaggacccgatgcccaaccccaggcctggcaagccctcggccccttccttggcccttggcccatccccaggagcctcgcccagctggagggctgcacccaaagcctcagacctgctgggggcccggggcccagggggaaccttcca

SEQ ID NO: 98 - DNA, 245 bp, Artificial Sequence
  0.25kb human FOXP3 3 primer HA designed for both TALEN and Cas9 approaches
gtgaggccctgggcccaggatggggcaggcagggtggggtacctggacctacaggtgccgacctttactgtggcactgggcgggaggggggctggctggggcacaggaagtggtttctgggtcccaggcaagtctgtgacttatgcagatgttgcagggccaagaaaatccccacctgccaggcctcagagattggaggctctccccgacctcccaatccctgtctcaggagaggaggaggccgt

SEQ ID NO: 99 - DNA, 300 bp, Artificial Sequence
  0.3kb human FOXP3 3 primer HA for Cas9-T9
gcctcgcccagctggagggctgcacccaaagcctcagacctgctgggggcccggggcccagggggaaccttccagggccgagatcttcgaggcggggcccatgcctcctcttcttccttgaaccccatgccaccatcgcagctgcaggtgaggccctgggcccaggatggggcaggcagggtggggtacctggacctacaggtgccgacctttactgtggcactgggcgggaggggggctggctggggcacaggaagtggtttctgggtcccaggcaagtctgtgacttatgcagatgtt

SEQ ID NO: 100 - DNA, 450 bp, Artificial Sequence
  0.45kb human FOXP3 3 primer HA for Cas9-T9
gcctcgcccagctggagggctgcacccaaagcctcagacctgctgggggcccggggcccagggggaaccttccagggccgagatcttcgaggcggggcccatgcctcctcttcttccttgaaccccatgccaccatcgcagctgcaggtgaggccctgggcccaggatggggcaggcagggtggggtacctggacctacaggtgccgacctttactgtggcactgggcgggaggggggctggctggggcacaggaagtggtttctgggtcccaggcaagtctgtgacttatgcagatgttgcagggccaagaaaatccccacctgccaggcctcagagattggaggctctccccgacctcccaatccctgtctcaggagaggaggaggccgtattgtagtcccatgagcatagctatgtgtccccatccccatgtgacaagagaagagga

SEQ ID NO: 101 - DNA, 600 bp, Artificial Sequence
  0.6kb human FOXP3 3 primer HA for Cas9-T9
gcctcgcccagctggagggctgcacccaaagcctcagacctgctgggggcccggggcccagggggaaccttccagggccgagatcttcgaggcggggcccatgcctcctcttcttccttgaaccccatgccaccatcgcagctgcaggtgaggccctgggcccaggatggggcaggcagggtggggtacctggacctacaggtgccgacctttactgtggcactgggcgggaggggggctggctggggcacaggaagtggtttctgggtcccaggcaagtctgtgacttatgcagatgttgcagggccaagaaaatccccacctgccaggcctcagagattggaggctctccccgacctcccaatccctgtctcaggagaggaggaggccgtattgtagtcccatgagcatagctatgtgtccccatccccatgtgacaagagaagaggactggggccaagtaggtgaggtgacagggctgaggccagctctgcaacttattagctgtttgatctttaaaaagttactcgatctccatgagcctcagtttccatacgtgtaaaagggggatgatcatagcatctaccatgtgggcttgca

SEQ ID NO: 102 - DNA, 794 bp, Artificial Sequence
  0.8kb human FOXP3 3 primer HA for Cas9-T9
gcctcgcccagctggagggctgcacccaaagcctcagacctgctgggggcccggggcccagggggaaccttccagggccgagatcttcgaggcggggcccatgcctcctcttcttccttgaaccccatgccaccatcgcagctgcaggtgaggccctgggcccaggatggggcaggcagggtggggtacctggacctacaggtgccgacctttactgtggcactgggcgggaggggggctggctggggcacaggaagtggtttctgggtcccaggcaagtctgtgacttatgcagatgttgcagggccaagaaaatccccacctgccaggcctcagagattggaggctctccccgacctcccaatccctgtctcaggagaggaggaggccgtattgtagtcccatgagcatagctatgtgtccccatccccatgtgacaagagaagaggactggggccaagtaggtgaggtgacagggctgaggccagctctgcaacttattagctgtttgatctttaaaaagttactcgatctccatgagcctcagtttccatacgtgtaaaagggggatgatcatagcatctaccatgtgggcttgcagtgcagagtatttgaattagacacagaacagtgaggatcaggatggcctctcacccacctgcctttctgcccagctgcccacactgcccctagtcatggtggcaccctccggggcacggctgggccccttgccccacttacaggcactcctccaggacaggccacatttcatgcaccaggtatggacggtgaat

SEQ ID NO: 103 - DNA, 300 bp, Artificial Sequence
  0.3kb human FOXP3 3 primer HA for Cas9-T3
cgagatcttcgaggcggggcccatgcctcctcttcttccttgaaccccatgccaccatcgcagctgcaggtgaggccctgggcccaggatggggcaggcagggtggggtacctggacctacaggtgccgacctttactgtggcactgggcgggaggggggctggctggggcacaggaagtggtttctgggtcccaggcaagtctgtgacttatgcagatgttgcagggccaagaaaatccccacctgccaggcctcagagattggaggctctccccgacctcccaatccctgtctcagga

SEQ ID NO: 104 - DNA, 451 bp, Artificial Sequence
  0.45kb human FOXP3 3 primer HA for Cas9-T3
cgagatcttcgaggcggggcccatgcctcctcttcttccttgaaccccatgccaccatcgcagctgcaggtgaggccctgggcccaggatggggcaggcagggtggggtacctggacctacaggtgccgacctttactgtggcactgggcgggaggggggctggctggggcacaggaagtggtttctgggtcccaggcaagtctgtgacttatgcagatgttgcagggccaagaaaatccccacctgccaggcctcagagattggaggctctccccgacctcccaatccctgtctcaggagaggaggaggccgtattgtagtcccatgagcatagctatgtgtccccatccccatgtgacaagagaagaggactggggccaagtaggtgaggtgacagggctgaggccagctctgcaacttattagctgtttgatctttaaaaagttactc

SEQ ID NO: 105 - DNA, 600 bp, Artificial Sequence
  0.6kb human FOXP3 3 primer HA for Cas9-T3
cgagatcttcgaggcggggcccatgcctcctcttcttccttgaaccccatgccaccatcgcagctgcaggtgaggccctgggcccaggatggggcaggcagggtggggtacctggacctacaggtgccgacctttactgtggcactgggcgggaggggggctggctggggcacaggaagtggtttctgggtcccaggcaagtctgtgacttatgcagatgttgcagggccaagaaaatccccacctgccaggcctcagagattggaggctctccccgacctcccaatccctgtctcaggagaggaggaggccgtattgtagtcccatgagcatagctatgtgtccccatccccatgtgacaagagaagaggactggggccaagtaggtgaggtgacagggctgaggccagctctgcaacttattagctgtttgatctttaaaaagttactcgatctccatgagcctcagtttccatacgtgtaaaagggggatgatcatagcatctaccatgtgggcttgcagtgcagagtatttgaattagacacagaacagtgaggatcaggatggcctctcacccacctgcctttctgcccagctgc

SEQ ID NO: 106 - DNA, 250 bp, Artificial Sequence
  0.25kb AAVS1 5 primer HA for Cas9-P1 and Cas9-N2
tagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgt

SEQ ID NO: 107 - DNA, 600 bp, Artificial Sequence
  0.6kb AAVS1 5 primer HA for Cas9-P1 and Cas9-N2
aggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgt

SEQ ID NO: 108 - DNA, 250 bp, Artificial Sequence
  0.25kb AAVS1 3 primer HA for Cas9-P1 and Cas9-N2
ctctggttctgggtacttttatctgtcccctccaccccacagtggggccactagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggaggga

SEQ ID NO: 109 - DNA, 600 bp, Artificial Sequence
  0.6kb AAVS1 3 primer HA for Cas9-P1 and Cas9-N2
ctctggttctgggtacttttatctgtcccctccaccccacagtggggccactagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggccgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccc

SEQ ID NO: 110 - protein, 273 aa, Artificial Sequence
  LNGFRt protein sequence
GAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKR

SEQ ID NO: 111 - protein, 157 aa, Artificial Sequence
  RQR8 protein sequence
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVV

SEQ ID NO: 112 - protein, 357 aa, Artificial Sequence
  EGFRt with GM-CSFR signal peptide
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 113 - DNA, 347 bp, Artificial Sequence
  MND promoter
gaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatc

SEQ ID NO: 114 - DNA, 523 bp, Artificial Sequence
  PGK promoter
ccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcggggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggggatcc

SEQ ID NO: 115 - DNA, 231 bp, Artificial Sequence
  EF1 promoter
aggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacaca

SEQ ID NO: 116 - DNA, 135 bp, Artificial Sequence
  SV40 polyA
tgctttatttgtgaaatttgtgatgctattgctttatttgtaaccattataagctgcaataaacaagttaacaacaacaattgcattcattttatgtttcaggttcagggggagatgtgggaggttttttaaagc

SEQ ID NO: 117 - DNA, 898 bp, Artificial Sequence
  3 primer UTR of FOXP3
cctcaagatcaaggaaaggaggatggacgaacaggggccaaactggtgggaggcagaggtggtgggggcagggatgataggccctggatgtgcccacagggaccaagaagtgaggtttccactgtcttgcctgccagggcccctgttcccccgctggcagccaccccctcccccatcatatcctttgccccaaggctgctcagaggggccccggtcctggccccagcccccacctccgccccagacacaccccccagtcgagccctgcagccaaacagagccttcacaaccagccacacagagcctgcctcagctgctcgcacagattacttcagggctggaaaagtcacacagacacacaaaatgtcacaatcctgtccctcactcaacacaaaccccaaaacacagagagcctgcctcagtacactcaaacaacctcaaagctgcatcatcacacaatcacacacaagcacagccctgacaacccacacaccccaaggcacgcacccacagccagcctcagggcccacaggggcactgtcaacacaggggtgtgcccagaggcctacacagaagcagcgtcagtaccctcaggatctgaggtcccaacacgtgctcgctcacacacacggcctgttagaattcacctgtgtatctcacgcatatgcacacgcacagccccccagtgggtctcttgagtcccgtgcagacacacacagccacacacactgccttgccaaaaataccccgtgtctcccctgccactcacctcactcccattccctgagccctgatccatgcctcagcttagactgcagaggaactactcatttatttgggatccaaggcccccaacccacagtaccgtccccaataaactgcagccgagctccccaca

SEQ ID NO: 118 - DNA, 822 bp, Artificial Sequence
  LNGFR coding sequence without stop codon
atgggggcaggtgccaccggacgagccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagagg

SEQ ID NO: 119 - DNA, 1899 bp, Artificial Sequence; identical to SEQ ID NO: 44
  microDISC: microCISC-FRB; nucleotide sequence

SEQ ID NO: 120 - protein, 633 aa, Artificial Sequence; identical to SEQ ID NO: 78
  mircoDISC: microCISC-FRB amino acid sequence

SEQ ID NO: 121 - DNA, 267 bp, Artificial Sequence
  FRB; nucleotide sequence
gagatgtggcatgagggtctggaagaagcgtctcgactgtactttggtgagcgcaatgtgaagggcatgtttgaagtcctcgaaccccttcatgccatgatggaacgcggaccccagaccttgaaggagacaagttttaaccaagcttacggaagagacctgatggaagcccaggaatggtgcaggaaatacatgaaaagcgggaatgtgaaggacttgctccaagcgtgggacctgtactatcatgtctttaggcgcattagtaag

SEQ ID NO: 122 - protein, 89 aa, Artificial Sequence
  FRB amino acid sequence
EMWHEGLEEASRLYFGERNVKGMFEVLEPLHAMMERGPQTLKETSFNQAYGRDLMEAQEWCRKYMKSGNVKDLLQAWDLYYHVFRRISK

SEQ ID NO: 123 - DNA, 1299 bp, Artificial Sequence
  CISCbeta: FRB-IL2Rbeta nucleotide sequence
atggcactgcccgtgaccgccctgctgctgcctctggccctgctgctgcacgcagcccggcctatcctgtggcacgagatgtggcacgagggcctggaggaggccagcaggctgtattttggcgagcgcaacgtgaagggcatgttcgaggtgctggagcctctgcacgccatgatggagagaggcccacagaccctgaaggagacatcctttaaccaggcctatggacgggacctgatggaggcacaggagtggtgcagaaagtacatgaagtctggcaatgtgaaggacctgctgcaggcctgggatctgtactatcacgtgtttcggagaatctccaagccagcagctctcggcaaagacacgattccgtggcttgggcatctgctcgttgggctgagcggtgcgtttggtttcatcatcttggtctatctcttgatcaattgcagaaatacaggcccttggctgaaaaaagtgctcaagtgtaatacccccgacccaagcaagttcttctcccagctttcttcagagcatggaggcgatgtgcagaaatggctctcttcaccttttccctcctcaagcttctccccgggagggctggcgcccgagatttcacctcttgaggtacttgaacgagacaaggttacccaacttctccttcaacaggataaggtacccgaacctgcgagccttagctccaaccactctcttacgagctgcttcaccaatcagggatacttctttttccaccttcccgatgcgctggaaatcgaagcttgtcaagtttactttacctatgatccatatagcgaggaagatcccgacgaaggagtcgccggtgcgcccacgggttcctcaccccaacctctccagcctctctcaggagaagatgatgcttattgcacttttcccagtagagacgatctcctcctcttttctccatctcttttggggggaccttcccccccttctacggcacctggcgggtctggtgctggcgaggagcggatgccgccgtccctccaggagcgagtaccacgagattgggatccccagccacttggaccccccacccccggcgtacctgaccttgtcgattttcaacctccccctgaattggtgctgcgagaggctggggaggaagttccggacgctgggccgagggagggcgtgtcctttccatggagtaggcctccaggtcaaggcgagtttagggctctcaacgcgcggctgccgttgaatacagacgcttatctctcactgcaggaactgcaaggtcaggacccaacacatcttgta

SEQ ID NO: 124 - protein, 433 aa, Artificial Sequence
  CISCbeta: FRB-IL2Rbeta amino acid sequence
MALPVTALLLPLALLLHAARPILWHEMWHEGLEEASRLYFGERNVKGMFEVLEPLHAMMERGPQTLKETSFNQAYGRDLMEAQEWCRKYMKSGNVKDLLQAWDLYYHVFRRISKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 125 - DNA, 20 bp, Artificial Sequence
  TCRa guide 1
atgcaagcccataaccgctg

SEQ ID NO: 126 - DNA, 20 bp, Artificial Sequence
  TCRa guide 2
caagaggccacagcggttat

SEQ ID NO: 127 - DNA, 20 bp, Artificial Sequence
  TCRa guide 3
ccaagaggccacagcggtta

SEQ ID NO: 128 - DNA, 20 bp, Artificial Sequence
  TCRa guide 4
ttcggaacccaatcactgac

SEQ ID NO: 129 - DNA, 20 bp, Artificial Sequence
  primer mix for insert forward
ggcacctccagaacaagacc

SEQ ID NO: 130 - DNA, 24 bp, Artificial Sequence
  primer mix for insert reverse
tcctgatcctcactgttctgtgtc

SEQ ID NO: 131 - DNA, 20 bp, Artificial Sequence
  primer mix for insert probe-FAM
agacccacaaccacagcagc

SEQ ID NO: 132 - DNA, 20 bp, Artificial Sequence
  primer mix for control forward
gttcacacgcatgtttgcct

SEQ ID NO: 133 - DNA, 20 bp, Artificial Sequence
  primer mix for control reverse
atcctgagggtactgacgct

SEQ ID NO: 134 - DNA, 19 bp, Artificial Sequence
  primer mix for control probe-Hex
tggcggtgactgggatggc

SEQ ID NO: 135 - DNA, 7342 bp, Artificial Sequence
  3017 pAAV_FOXP3.025_MND.FOXP3geneartCDS.P2A.GFP.WPRE3.pA_025
gtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcggccgctgctagcgtgggcaggcaagccaggtgctggacctctgcacgtggggcatgtgtgggtatgtacatgtacctgtgttcttggtgtgtgtgtgtgtgtgtgtgtgtgtgtgtgtctagagctggggtgcaactatggggcccctcgggacatgtcccagccaatgcctgctttgaccagaggagtgtccacgtggctcaggtggtcgagtatctcataccgccctagcacacgtgtgactcctttcccctattgtctacacgcgtaggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggatctcgaggccaccatgcctaatcctcggcctggaaagcctagcgctccttctcttgctctgggaccttctcctggcgcctctccatcttggagagccgctcctaaagccagcgatctgctgggagctagaggacctggcggcacatttcagggcagagatcttagaggcggagcccacgctagctcctccagccttaatcctatgcctcctagccagctccagctgcctacactgcctctggttatggtggctcctagcggagctagactgggccctctgcctcatctgcaagctctgctgcaggacagaccccacttcatgcaccagctgagcaccgtggatgcccacgcaagaacacctgtgctgcaggttcaccctctggaatccccagccatgatcagcctgacacctccaacaacagccaccggcgtgttcagcctgaaagccagacctggactgcctcctggcatcaatgtggccagcctggaatgggtgtccagagaacctgctctgctgtgcacattccccaatccaagcgctcccagaaaggacagcacactgtctgccgtgcctcagagcagctatcccctgcttgctaacggcgtgtgcaagtggcctggatgcgagaaggtgttcgaggaacccgaggacttcctgaagcactgccaggccgatcatctgctggacgagaaaggcagagcccagtgtctgctccagcgcgagatggtgcagtctctggaacagcagctggtcctggaaaaagaaaagctgagcgccatgcaggcccacctggccggaaaaatggccctgacaaaggccagcagcgtggcctcttctgataagggcagctgctgcattgtggccgctggatctcagggacctgtggttcctgcttggagcggacctagagaggcccctgattctctgtttgccgtgcggagacacctgtggggctctcacggcaactctactttccccgagttcctgcacaacatggactacttcaagttccacaacatgcggcctccattcacctacgccacactgatcagatgggccattctggaagcccctgagaagcagagaaccctgaacgagatctaccactggtttacccggatgttcgccttcttccggaatcaccctgccacctggaagaacgccatccggcacaatctgagcctgcacaagtgcttcgtgcgcgtggaatctgagaaaggcgccgtgtggacagtggacgagctggaattcagaaagaagagaagccagcggcctagccggtgcagcaatcctacacctggacctggaagcggagcgactaacttcagcctgctgaagcaggccggagatgtggaggaaaaccctggaccgatggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgaaagcttccacggaattgtcagtgcccaacagccgagcccctgtccagcagcgggcaaggcaggcggcgatgagttccgccgtggcaagaactaaccaggatttatacaaggaggagaaaatgaaagccatacgggaagcaatagcatgatacaaaggcattaaagcagcgtatccacatagcgtaaaaggagcaacatagttaagaataccagtcaatctttcacaaattttgtaatccagaggttgattatcgtcgactgctttatttgtgaaatttgtgatgctattgctttatttgtaaccattataagctgcaataaacaagttaacaacaacaattgcattcattttatgtttcaggttcagggggagatgtgggaggttttttaaagcactagtgtgaggccctgggcccaggatggggcaggcagggtggggtacctggacctacaggtgccgacctttactgtggcactgggcgggaggggggctggctggggcacaggaagtggtttctgggtcccaggcaagtctgtgacttatgcagatgttgcagggccaagaaaatccccacctgccaggcctcagagattggaggctctccccgacctcccaatccctgtctcaggagaggaggaggccgtggatcctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagacccc

SEQ ID NO: 136 - DNA, 7694 bp, Artificial Sequence
  3018_pAAV_FOXP3.025_MND.FOXP3geneartCDS.P2A.GFP.WPRE6.pA_025
gtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcggccgctgctagcgtgggcaggcaagccaggtgctggacctctgcacgtggggcatgtgtgggtatgtacatgtacctgtgttcttggtgtgtgtgtgtgtgtgtgtgtgtgtgtgtgtctagagctggggtgcaactatggggcccctcgggacatgtcccagccaatgcctgctttgaccagaggagtgtccacgtggctcaggtggtcgagtatctcataccgccctagcacacgtgtgactcctttcccctattgtctacacgcgtaggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggatctcgaggccaccatgcctaatcctcggcctggaaagcctagcgctccttctcttgctctgggaccttctcctggcgcctctccatcttggagagccgctcctaaagccagcgatctgctgggagctagaggacctggcggcacatttcagggcagagatcttagaggcggagcccacgctagctcctccagccttaatcctatgcctcctagccagctccagctgcctacactgcctctggttatggtggctcctagcggagctagactgggccctctgcctcatctgcaagctctgctgcaggacagaccccacttcatgcaccagctgagcaccgtggatgcccacgcaagaacacctgtgctgcaggttcaccctctggaatccccagccatgatcagcctgacacctccaacaacagccaccggcgtgttcagcctgaaagccagacctggactgcctcctggcatcaatgtggccagcctggaatgggtgtccagagaacctgctctgctgtgcacattccccaatccaagcgctcccagaaaggacagcacactgtctgccgtgcctcagagcagctatcccctgcttgctaacggcgtgtgcaagtggcctggatgcgagaaggtgttcgaggaacccgaggacttcctgaagcactgccaggccgatcatctgctggacgagaaaggcagagcccagtgtctgctccagcgcgagatggtgcagtctctggaacagcagctggtcctggaaaaagaaaagctgagcgccatgcaggcccacctggccggaaaaatggccctgacaaaggccagcagcgtggcctcttctgataagggcagctgctgcattgtggccgctggatctcagggacctgtggttcctgcttggagcggacctagagaggcccctgattctctgtttgccgtgcggagacacctgtggggctctcacggcaactctactttccccgagttcctgcacaacatggactacttcaagttccacaacatgcggcctccattcacctacgccacactgatcagatgggccattctggaagcccctgagaagcagagaaccctgaacgagatctaccactggtttacccggatgttcgccttcttccggaatcaccctgccacctggaagaacgccatccggcacaatctgagcctgcacaagtgcttcgtgcgcgtggaatctgagaaaggcgccgtgtggacagtggacgagctggaattcagaaagaagagaagccagcggcctagccggtgcagcaatcctacacctggacctggaagcggagcgactaacttcagcctgctgaagcaggccggagatgtggaggaaaaccctggaccgatggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgaaagctttcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggagtcgactgctttatttgtgaaatttgtgatgctattgctttatttgtaaccattataagctgcaataaacaagttaacaacaacaattgcattcattttatgtttcaggttcagggggagatgtgggaggttttttaaagcactagtgtgaggccctgggcccaggatggggcaggcagggtggggtacctggacctacaggtgccgacctttactgtggcactgggcgggaggggggctggctggggcacaggaagtggtttctgggtcccaggcaagtctgtgacttatgcagatgttgcagggccaagaaaatccccacctgccaggcctcagagattggaggctctccccgacctcccaatccctgtctcaggagaggaggaggccgtggatcctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagacccc

SEQ ID NO: 137 - DNA, 7342 bp, Artificial Sequence
  3019 pAAV_FOXP3.025_MND.FOXP3geneartCDS.P2A.GFP.WPREc3.pA_025
gtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcggccgctgctagcgtgggcaggcaagccaggtgctggacctctgcacgtggggcatgtgtgggtatgtacatgtacctgtgttcttggtgtgtgtgtgtgtgtgtgtgtgtgtgtgtgtctagagctggggtgcaactatggggcccctcgggacatgtcccagccaatgcctgctttgaccagaggagtgtccacgtggctcaggtggtcgagtatctcataccgccctagcacacgtgtgactcctttcccctattgtctacacgcgtaggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggatctcgaggccaccatgcctaatcctcggcctggaaagcctagcgctccttctcttgctctgggaccttctcctggcgcctctccatcttggagagccgctcctaaagccagcgatctgctgggagctagaggacctggcggcacatttcagggcagagatcttagaggcggagcccacgctagctcctccagccttaatcctatgcctcctagccagctccagctgcctacactgcctctggttatggtggctcctagcggagctagactgggccctctgcctcatctgcaagctctgctgcaggacagaccccacttcatgcaccagctgagcaccgtggatgcccacgcaagaacacctgtgctgcaggttcaccctctggaatccccagccatgatcagcctgacacctccaacaacagccaccggcgtgttcagcctgaaagccagacctggactgcctcctggcatcaatgtggccagcctggaatgggtgtccagagaacctgctctgctgtgcacattccccaatccaagcgctcccagaaaggacagcacactgtctgccgtgcctcagagcagctatcccctgcttgctaacggcgtgtgcaagtggcctggatgcgagaaggtgttcgaggaacccgaggacttcctgaagcactgccaggccgatcatctgctggacgagaaaggcagagcccagtgtctgctccagcgcgagatggtgcagtctctggaacagcagctggtcctggaaaaagaaaagctgagcgccatgcaggcccacctggccggaaaaatggccctgacaaaggccagcagcgtggcctcttctgataagggcagctgctgcattgtggccgctggatctcagggacctgtggttcctgcttggagcggacctagagaggcccctgattctctgtttgccgtgcggagacacctgtggggctctcacggcaactctactttccccgagttcctgcacaacatggactacttcaagttccacaacatgcggcctccattcacctacgccacactgatcagatgggccattctggaagcccctgagaagcagagaaccctgaacgagatctaccactggtttacccggatgttcgccttcttccggaatcaccctgccacctggaagaacgccatccggcacaatctgagcctgcacaagtgcttcgtgcgcgtggaatctgagaaaggcgccgtgtggacagtggacgagctggaattcagaaagaagagaagccagcggcctagccggtgcagcaatcctacacctggacctggaagcggagcgactaacttcagcctgctgaagcaggccggagatgtggaggaaaaccctggaccgatggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgaaagcttgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtgggtcgactgctttatttgtgaaatttgtgatgctattgctttatttgtaaccattataagctgcaataaacaagttaacaacaacaattgcattcattttatgtttcaggttcagggggagatgtgggaggttttttaaagcactagtgtgaggccctgggcccaggatggggcaggcagggtggggtacctggacctacaggtgccgacctttactgtggcactgggcgggaggggggctggctggggcacaggaagtggtttctgggtcccaggcaagtctgtgacttatgcagatgttgcagggccaagaaaatccccacctgccaggcctcagagattggaggctctccccgacctcccaatccctgtctcaggagaggaggaggccgtggatcctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagacccc

SEQ ID NO: 138 - DNA, 7989 bp, Artificial Sequence
  3020 pAAV_FOXP3.045_MND.FOXP3geneartCDS.P2A.LNGFR.WPRE3.pA_06
gtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcggccgcagtccatgcctagtcactggggcaaaataggactccgaggagaaagtccgagaccagctccggcaagatgagcaaacacagcctgtgcagggtgcagggagggctagaggcctgaggcttgaaacagctctcaagtggagggggaaacaaccattgccctcatagaggacacatccacaccagggctgtgctagcgtgggcaggcaagccaggtgctggacctctgcacgtggggcatgtgtgggtatgtacatgtacctgtgttcttggtgtgtgtgtgtgtgtgtgtgtgtgtgtgtgtctagagctggggtgcaactatggggcccctcgggacatgtcccagccaatgcctgctttgaccagaggagtgtccacgtggctcaggtggtcgagtatctcataccgccctagcacacgtgtgactcctttcccctattgtctacacgcgtaggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggatctcgaggccaccatgcctaatcctcggcctggaaagcctagcgctccttctcttgctctgggaccttctcctggcgcctctccatcttggagagccgctcctaaagccagcgatctgctgggagctagaggacctggcggcacatttcagggcagagatcttagaggcggagcccacgctagctcctccagccttaatcctatgcctcctagccagctccagctgcctacactgcctctggttatggtggctcctagcggagctagactgggccctctgcctcatctgcaagctctgctgcaggacagaccccacttcatgcaccagctgagcaccgtggatgcccacgcaagaacacctgtgctgcaggttcaccctctggaatccccagccatgatcagcctgacacctccaacaacagccaccggcgtgttcagcctgaaagccagacctggactgcctcctggcatcaatgtggccagcctggaatgggtgtccagagaacctgctctgctgtgcacattccccaatccaagcgctcccagaaaggacagcacactgtctgccgtgcctcagagcagctatcccctgcttgctaacggcgtgtgcaagtggcctggatgcgagaaggtgttcgaggaacccgaggacttcctgaagcactgccaggccgatcatctgctggacgagaaaggcagagcccagtgtctgctccagcgcgagatggtgcagtctctggaacagcagctggtcctggaaaaagaaaagctgagcgccatgcaggcccacctggccggaaaaatggccctgacaaaggccagcagcgtggcctcttctgataagggcagctgctgcattgtggccgctggatctcagggacctgtggttcctgcttggagcggacctagagaggcccctgattctctgtttgccgtgcggagacacctgtggggctctcacggcaactctactttccccgagttcctgcacaacatggactacttcaagttccacaacatgcggcctccattcacctacgccacactgatcagatgggccattctggaagcccctgagaagcagagaaccctgaacgagatctaccactggtttacccggatgttcgccttcttccggaatcaccctgccacctggaagaacgccatccggcacaatctgagcctgcacaagtgcttcgtgcgcgtggaatctgagaaaggcgccgtgtggacagtggacgagctggaattcagaaagaagagaagccagcggcctagccggtgcagcaatcctacacctggacctggaagcggagcgactaacttcagcctgctgaagcaggccggagatgtggaggaaaaccctggaccgatgggggcaggtgccaccggacgagccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggtgaaagcttccacggaattgtcagtgcccaacagccgagcccctgtccagcagcgggcaaggcaggcggcgatgagttccgccgtggcaagaactaaccaggatttatacaaggaggagaaaatgaaagccatacgggaagcaatagcatgatacaaaggcattaaagcagcgtatccacatagcgtaaaaggagcaacatagttaagaataccagtcaatctttcacaaattttgtaatccagaggttgattatcgtcgactgctttatttgtgaaatttgtgatgctattgctttatttgtaaccattataagctgcaataaacaagttaacaacaacaattgcattcattttatgtttcaggttcagggggagatgtgggaggttttttaaagcactagtgtgaggccctgggcccaggatggggcaggcagggtggggtacctggacctacaggtgccgacctttactgtggcactgggcgggaggggggctggctggggcacaggaagtggtttctgggtcccaggcaagtctgtgacttatgcagatgttgcagggccaagaaaatccccacctgccaggcctcagagattggaggctctccccgacctcccaatccctgtctcaggagaggaggaggccgtattgtagtcccatgagcatagctatgtgtccccatccccatgtgacaagagaagaggactggggccaagtaggtgaggtgacagggctgaggccagctctgcaacttattagctgtttgatctttaaaaagttactcgatctccatgagcctcagtttccatacgtgtaaaagggggatgatcatagcatctaccatgtgggcttgcagtgcagagtatttgaattagacacagaacagtgaggatcaggatggcctctcacccacctgcctttctgcccagctgcccacactgcccctagtcatggtggcaccctccggggcacggctgggccccttgccccacttacaggcaccgcggcgctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagacccc

SEQ ID NO: 139 - DNA, 7444 bp, Artificial Sequence
  3021 pAAV_FOXP3.025_MND.FOXP3geneartCDS.P2A.LNGFR.WPRE3.pA_025
gtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcggccgctgctagcgtgggcaggcaagccaggtgctggacctctgcacgtggggcatgtgtgggtatgtacatgtacctgtgttcttggtgtgtgtgtgtgtgtgtgtgtgtgtgtgtgtctagagctggggtgcaactatggggcccctcgggacatgtcccagccaatgcctgctttgaccagaggagtgtccacgtggctcaggtggtcgagtatctcataccgccctagcacacgtgtgactcctttcccctattgtctacacgcgtaggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggatctcgaggccaccatgcctaatcctcggcctggaaagcctagcgctccttctcttgctctgggaccttctcctggcgcctctccatcttggagagccgctcctaaagccagcgatctgctgggagctagaggacctggcggcacatttcagggcagagatcttagaggcggagcccacgctagctcctccagccttaatcctatgcctcctagccagctccagctgcctacactgcctctggttatggtggctcctagcggagctagactgggccctctgcctcatctgcaagctctgctgcaggacagaccccacttcatgcaccagctgagcaccgtggatgcccacgcaagaacacctgtgctgcaggttcaccctctggaatccccagccatgatcagcctgacacctccaacaacagccaccggcgtgttcagcctgaaagccagacctggactgcctcctggcatcaatgtggccagcctggaatgggtgtccagagaacctgctctgctgtgcacattccccaatccaagcgctcccagaaaggacagcacactgtctgccgtgcctcagagcagctatcccctgcttgctaacggcgtgtgcaagtggcctggatgcgagaaggtgttcgaggaacccgaggacttcctgaagcactgccaggccgatcatctgctggacgagaaaggcagagcccagtgtctgctccagcgcgagatggtgcagtctctggaacagcagctggtcctggaaaaagaaaagctgagcgccatgcaggcccacctggccggaaaaatggccctgacaaaggccagcagcgtggcctcttctgataagggcagctgctgcattgtggccgctggatctcagggacctgtggttcctgcttggagcggacctagagaggcccctgattctctgtttgccgtgcggagacacctgtggggctctcacggcaactctactttccccgagttcctgcacaacatggactacttcaagttccacaacatgcggcctccattcacctacgccacactgatcagatgggccattctggaagcccctgagaagcagagaaccctgaacgagatctaccactggtttacccggatgttcgccttcttccggaatcaccctgccacctggaagaacgccatccggcacaatctgagcctgcacaagtgcttcgtgcgcgtggaatctgagaaaggcgccgtgtggacagtggacgagctggaattcagaaagaagagaagccagcggcctagccggtgcagcaatcctacacctggacctggaagcggagcgactaacttcagcctgctgaagcaggccggagatgtggaggaaaaccctggaccgatgggggcaggtgccaccggacgagccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggtgaaagcttccacggaattgtcagtgcccaacagccgagcccctgtccagcagcgggcaaggcaggcggcgatgagttccgccgtggcaagaactaaccaggatttatacaaggaggagaaaatgaaagccatacgggaagcaatagcatgatacaaaggcattaaagcagcgtatccacatagcgtaaaaggagcaacatagttaagaataccagtcaatctttcacaaattttgtaatccagaggttgattatcgtcgactgctttatttgtgaaatttgtgatgctattgctttatttgtaaccattataagctgcaataaacaagttaacaacaacaattgcattcattttatgtttcaggttcagggggagatgtgggaggttttttaaagcactagtgtgaggccctgggcccaggatggggcaggcagggtggggtacctggacctacaggtgccgacctttactgtggcactgggcgggaggggggctggctggggcacaggaagtggtttctgggtcccaggcaagtctgtgacttatgcagatgttgcagggccaagaaaatccccacctgccaggcctcagagattggaggctctccccgacctcccaatccctgtctcaggagaggaggaggccgtggatcctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagacccc

SEQ ID NO: 140 - DNA, 7342 bp, Artificial Sequence; identical to SEQ ID NO: 135
  3017 pAAV_FOXP3.025_MND.FOXP3geneartCDS.P2A.GFP.WPRE3.pA_025

SEQ ID NO: 141 - DNA, 7694 bp, Artificial Sequence; identical to SEQ ID NO: 136
  3018_pAAV_FOXP3.025_MND.FOXP3geneartCDS.P2A.GFP.WPRE6.pA_025

SEQ ID NO: 142 - DNA, 7342 bp, Artificial Sequence; identical to SEQ ID NO: 137
  3019 pAAV_FOXP3.025_MND.FOXP3geneartCDS.P2A.GFP.WPREc3.pA_025

SEQ ID NO: 143 - DNA, 7989 bp, Artificial Sequence; identical to SEQ ID NO: 138
  3020 pAAV_FOXP3.045_MND.FOXP3geneartCDS.P2A.LNGFR.WPRE3.pA_06

SEQ ID NO: 144 - DNA, 7444 bp, Artificial Sequence; identical to SEQ ID NO: 139
  3021 pAAV_FOXP3.025_MND.FOXP3geneartCDS.P2A.LNGFR.WPRE3.pA_025

SEQ ID NO: 145 - DNA, 7444 bp, Artificial Sequence
  3022 pAAV_FOXP3.025_MND.FOXP3geneartCDS.P2A.LNGFR.WPREc3.pA_025
gtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcggccgctgctagcgtgggcaggcaagccaggtgctggacctctgcacgtggggcatgtgtgggtatgtacatgtacctgtgttcttggtgtgtgtgtgtgtgtgtgtgtgtgtgtgtgtctagagctggggtgcaactatggggcccctcgggacatgtcccagccaatgcctgctttgaccagaggagtgtccacgtggctcaggtggtcgagtatctcataccgccctagcacacgtgtgactcctttcccctattgtctacacgcgtaggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggatctcgaggccaccatgcctaatcctcggcctggaaagcctagcgctccttctcttgctctgggaccttctcctggcgcctctccatcttggagagccgctcctaaagccagcgatctgctgggagctagaggacctggcggcacatttcagggcagagatcttagaggcggagcccacgctagctcctccagccttaatcctatgcctcctagccagctccagctgcctacactgcctctggttatggtggctcctagcggagctagactgggccctctgcctcatctgcaagctctgctgcaggacagaccccacttcatgcaccagctgagcaccgtggatgcccacgcaagaacacctgtgctgcaggttcaccctctggaatccccagccatgatcagcctgacacctccaacaacagccaccggcgtgttcagcctgaaagccagacctggactgcctcctggcatcaatgtggccagcctggaatgggtgtccagagaacctgctctgctgtgcacattccccaatccaagcgctcccagaaaggacagcacactgtctgccgtgcctcagagcagctatcccctgcttgctaacggcgtgtgcaagtggcctggatgcgagaaggtgttcgaggaacccgaggacttcctgaagcactgccaggccgatcatctgctggacgagaaaggcagagcccagtgtctgctccagcgcgagatggtgcagtctctggaacagcagctggtcctggaaaaagaaaagctgagcgccatgcaggcccacctggccggaaaaatggccctgacaaaggccagcagcgtggcctcttctgataagggcagctgctgcattgtggccgctggatctcagggacctgtggttcctgcttggagcggacctagagaggcccctgattctctgtttgccgtgcggagacacctgtggggctctcacggcaactctactttccccgagttcctgcacaacatggactacttcaagttccacaacatgcggcctccattcacctacgccacactgatcagatgggccattctggaagcccctgagaagcagagaaccctgaacgagatctaccactggtttacccggatgttcgccttcttccggaatcaccctgccacctggaagaacgccatccggcacaatctgagcctgcacaagtgcttcgtgcgcgtggaatctgagaaaggcgccgtgtggacagtggacgagctggaattcagaaagaagagaagccagcggcctagccggtgcagcaatcctacacctggacctggaagcggagcgactaacttcagcctgctgaagcaggccggagatgtggaggaaaaccctggaccgatgggggcaggtgccaccggacgagccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggtgaaagcttgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtgggtcgactgctttatttgtgaaatttgtgatgctattgctttatttgtaaccattataagctgcaataaacaagttaacaacaacaattgcattcattttatgtttcaggttcagggggagatgtgggaggttttttaaagcactagtgtgaggccctgggcccaggatggggcaggcagggtggggtacctggacctacaggtgccgacctttactgtggcactgggcgggaggggggctggctggggcacaggaagtggtttctgggtcccaggcaagtctgtgacttatgcagatgttgcagggccaagaaaatccccacctgccaggcctcagagattggaggctctccccgacctcccaatccctgtctcaggagaggaggaggccgtggatcctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagacccc

SEQ ID NO: 146 - DNA, 7989 bp, Artificial Sequence
  3023 pAAV_FOXP3.045_MND.FOXP3geneartCDS.P2A.LNGFR.WPREc3.pA_06
gtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcggccgcagtccatgcctagtcactggggcaaaataggactccgaggagaaagtccgagaccagctccggcaagatgagcaaacacagcctgtgcagggtgcagggagggctagaggcctgaggcttgaaacagctctcaagtggagggggaaacaaccattgccctcatagaggacacatccacaccagggctgtgctagcgtgggcaggcaagccaggtgctggacctctgcacgtggggcatgtgtgggtatgtacatgtacctgtgttcttggtgtgtgtgtgtgtgtgtgtgtgtgtgtgtgtctagagctggggtgcaactatggggcccctcgggacatgtcccagccaatgcctgctttgaccagaggagtgtccacgtggctcaggtggtcgagtatctcataccgccctagcacacgtgtgactcctttcccctattgtctacacgcgtaggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggatctcgaggccaccatgcctaatcctcggcctggaaagcctagcgctccttctcttgctctgggaccttctcctggcgcctctccatcttggagagccgctcctaaagccagcgatctgctgggagctagaggacctggcggcacatttcagggcagagatcttagaggcggagcccacgctagctcctccagccttaatcctatgcctcctagccagctccagctgcctacactgcctctggttatggtggctcctagcggagctagactgggccctctgcctcatctgcaagctctgctgcaggacagaccccacttcatgcaccagctgagcaccgtggatgcccacgcaagaacacctgtgctgcaggttcaccctctggaatccccagccatgatcagcctgacacctccaacaacagccaccggcgtgttcagcctgaaagccagacctggactgcctcctggcatcaatgtggccagcctggaatgggtgtccagagaacctgctctgctgtgcacattccccaatccaagcgctcccagaaaggacagcacactgtctgccgtgcctcagagcagctatcccctgcttgctaacggcgtgtgcaagtggcctggatgcgagaaggtgttcgaggaacccgaggacttcctgaagcactgccaggccgatcatctgctggacgagaaaggcagagcccagtgtctgctccagcgcgagatggtgcagtctctggaacagcagctggtcctggaaaaagaaaagctgagcgccatgcaggcccacctggccggaaaaatggccctgacaaaggccagcagcgtggcctcttctgataagggcagctgctgcattgtggccgctggatctcagggacctgtggttcctgcttggagcggacctagagaggcccctgattctctgtttgccgtgcggagacacctgtggggctctcacggcaactctactttccccgagttcctgcacaacatggactacttcaagttccacaacatgcggcctccattcacctacgccacactgatcagatgggccattctggaagcccctgagaagcagagaaccctgaacgagatctaccactggtttacccggatgttcgccttcttccggaatcaccctgccacctggaagaacgccatccggcacaatctgagcctgcacaagtgcttcgtgcgcgtggaatctgagaaaggcgccgtgtggacagtggacgagctggaattcagaaagaagagaagccagcggcctagccggtgcagcaatcctacacctggacctggaagcggagcgactaacttcagcctgctgaagcaggccggagatgtggaggaaaaccctggaccgatgggggcaggtgccaccggacgagccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggtgaaagcttgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtgggtcgactgctttatttgtgaaatttgtgatgctattgctttatttgtaaccattataagctgcaataaacaagttaacaacaacaattgcattcattttatgtttcaggttcagggggagatgtgggaggttttttaaagcactagtgtgaggccctgggcccaggatggggcaggcagggtggggtacctggacctacaggtgccgacctttactgtggcactgggcgggaggggggctggctggggcacaggaagtggtttctgggtcccaggcaagtctgtgacttatgcagatgttgcagggccaagaaaatccccacctgccaggcctcagagattggaggctctccccgacctcccaatccctgtctcaggagaggaggaggccgtattgtagtcccatgagcatagctatgtgtccccatccccatgtgacaagagaagaggactggggccaagtaggtgaggtgacagggctgaggccagctctgcaacttattagctgtttgatctttaaaaagttactcgatctccatgagcctcagtttccatacgtgtaaaagggggatgatcatagcatctaccatgtgggcttgcagtgcagagtatttgaattagacacagaacagtgaggatcaggatggcctctcacccacctgcctttctgcccagctgcccacactgcccctagtcatggtggcaccctccggggcacggctgggccccttgccccacttacaggcaccgcggcgctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagacccc

SEQ ID NO: 147 - DNA, 8341 bp, Artificial Sequence
  3024 pAAV_FOXP3_045_MND-FOXP3geneartCDS.P2A.LNGFR.WPRE6.pA_06
gtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcggccgcagtccatgcctagtcactggggcaaaataggactccgaggagaaagtccgagaccagctccggcaagatgagcaaacacagcctgtgcagggtgcagggagggctagaggcctgaggcttgaaacagctctcaagtggagggggaaacaaccattgccctcatagaggacacatccacaccagggctgtgctagcgtgggcaggcaagccaggtgctggacctctgcacgtggggcatgtgtgggtatgtacatgtacctgtgttcttggtgtgtgtgtgtgtgtgtgtgtgtgtgtgtgtctagagctggggtgcaactatggggcccctcgggacatgtcccagccaatgcctgctttgaccagaggagtgtccacgtggctcaggtggtcgagtatctcataccgccctagcacacgtgtgactcctttcccctattgtctacacgcgtaggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggatctcgaggccaccatgcctaatcctcggcctggaaagcctagcgctccttctcttgctctgggaccttctcctggcgcctctccatcttggagagccgctcctaaagccagcgatctgctgggagctagaggacctggcggcacatttcagggcagagatcttagaggcggagcccacgctagctcctccagccttaatcctatgcctcctagccagctccagctgcctacactgcctctggttatggtggctcctagcggagctagactgggccctctgcctcatctgcaagctctgctgcaggacagaccccacttcatgcaccagctgagcaccgtggatgcccacgcaagaacacctgtgctgcaggttcaccctctggaatccccagccatgatcagcctgacacctccaacaacagccaccggcgtgttcagcctgaaagccagacctggactgcctcctggcatcaatgtggccagcctggaatgggtgtccagagaacctgctctgctgtgcacattccccaatccaagcgctcccagaaaggacagcacactgtctgccgtgcctcagagcagctatcccctgcttgctaacggcgtgtgcaagtggcctggatgcgagaaggtgttcgaggaacccgaggacttcctgaagcactgccaggccgatcatctgctggacgagaaaggcagagcccagtgtctgctccagcgcgagatggtgcagtctctggaacagcagctggtcctggaaaaagaaaagctgagcgccatgcaggcccacctggccggaaaaatggccctgacaaaggccagcagcgtggcctcttctgataagggcagctgctgcattgtggccgctggatctcagggacctgtggttcctgcttggagcggacctagagaggcccctgattctctgtttgccgtgcggagacacctgtggggctctcacggcaactctactttccccgagttcctgcacaacatggactacttcaagttccacaacatgcggcctccattcacctacgccacactgatcagatgggccattctggaagcccctgagaagcagagaaccctgaacgagatctaccactggtttacccggatgttcgccttcttccggaatcaccctgccacctggaagaacgccatccggcacaatctgagcctgcacaagtgcttcgtgcgcgtggaatctgagaaaggcgccgtgtggacagtggacgagctggaattcagaaagaagagaagccagcggcctagccggtgcagcaatcctacacctggacctggaagcggagcgactaacttcagcctgctgaagcaggccggagatgtggaggaaaaccctggaccgatgggggcaggtgccaccggacgagccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggtgaaagctttcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggagtcgactgctttatttgtgaaatttgtgatgctattgctttatttgtaaccattataagctgcaataaacaagttaacaacaacaattgcattcattttatgtttcaggttcagggggagatgtgggaggttttttaaagcactagtgtgaggccctgggcccaggatggggcaggcagggtggggtacctggacctacaggtgccgacctttactgtggcactgggcgggaggggggctggctggggcacaggaagtggtttctgggtcccaggcaagtctgtgacttatgcagatgttgcagggccaagaaaatccccacctgccaggcctcagagattggaggctctccccgacctcccaatccctgtctcaggagaggaggaggccgtattgtagtcccatgagcatagctatgtgtccccatccccatgtgacaagagaagaggactggggccaagtaggtgaggtgacagggctgaggccagctctgcaacttattagctgtttgatctttaaaaagttactcgatctccatgagcctcagtttccatacgtgtaaaagggggatgatcatagcatctaccatgtgggcttgcagtgcagagtatttgaattagacacagaacagtgaggatcaggatggcctctcacccacctgcctttctgcccagctgcccacactgcccctagtcatggtggcaccctccggggcacggctgggccccttgccccacttacaggcaccgcggcgctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagacccc

SEQ ID NO: 148 - DNA, 7553 bp, Artificial Sequence
  1303 pAAV FOXP3_0.9[MND-GFPki]1.6
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgctcaagagaccccatctctcctcctctctgtcacttgccatgctggatccgtgcatgatcacactcctggactcgcctccttgccctgagatccagacccccgtattcagctgccccctcagctcctccactcacatatttaatgccagactcttcatgtctatctacacctgcacttttgcacccaatccaactccccgccatgtcccccatctcaggtaatgtcagctcggtccttccagctgctcaagctaaaacccatgtcactttgactctccctcttgcccactacatccaagctgctagcactgctcctgatccagcttcagattaagtctcagaatctacccacttctcgccttctccactgccaccagcccattctgtgccagcatcatcacttgccaggactgttacaatagcctcctcactagccccactcacagcagccagatgaatcttttgagtccatgcctagtcactggggcaaaataggactccgaggagaaagtccgagaccagctccggcaagatgagcaaacacagcctgtgcagggtgcagggagggctagaggcctgaggcttgaaacagctctcaagtggagggggaaacaaccattgccctcatagaggacacatccacaccagggctgtgctagcgtgggcaggcaagccaggtgctggacctctgcacgtggggcatgtgtgggtatgtacatgtacctgtgttcttggtgtgtgtgtgtgtgtgtgtgtgtgtgtgtgtctagagctggggtgcaactatggggcccctcgggacatgtcccagccaatgcctgctttgaccagaggagtgtccacgtggctcaggtggtcgagtatctcataccgccctagcacacgtgtgactcctttcccctattgtctacgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgtctacgcagcctgcccttggacaaggacccgatgcccaaccccaggcctgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggcaagccctcggccccttccttggcccttggcccatccccaggagcctcgcccagctggagggctgcccctaaagcaagcgacctgctgggggcccggggcccgggtggcacgttccagggccgagatcttcgaggcggggcccatgcctcctcttcttccttgaaccccatgccaccatcgcagctgcaggtgaggccctgggcccaggatggggcaggcagggtggggtacctggacctacaggtgccgacctttactgtggcactgggcgggaggggggctggctggggcacaggaagtggtttctgggtcccaggcaagtctgtgacttatgcagatgttgcagggccaagaaaatccccacctgccaggcctcagagattggaggctctccccgacctcccaatccctgtctcaggagaggaggaggccgtattgtagtcccatgagcatagctatgtgtccccatccccatgtgacaagagaagaggactggggccaagtaggtgaggtgacagggctgaggccagctctgcaacttattagctgtttgatctttaaaaagttactcgatctccatgagcctcagtttccatacgtgtaaaagggggatgatcatagcatctaccatgtgggcttgcagtgcagagtatttgaattagacacagaacagtgaggatcaggatggcctctcacccacctgcctttctgcccagctgcccacactgcccctagtcatggtggcaccctccggggcacggctgggccccttgccccacttacaggcactcctccaggacaggccacatttcatgcaccaggtatggacggtgaatgggcagggaggagggagcaggtgggagaactgtggggaggggccccgagtcaggctgaaccacagcccacatgtgccccccagctctcaacggtggatgcccacgcccggacccctgtgctgcaggtgcaccccctggagagcccagccatgatcagcctcacaccacccaccaccgccactggggtcttctccctcaaggcccggcctggcctcccacctggtaacacctcagcccgtaccccatggcttcacagaacccccaagtccccagatccttggctgtgagcagtgtaggctattctgaattgcagtactctgggggtcaaaggtgtcaggtctcagaggcttggaaactccaccctccaaaaaacgtcaggtgcagaaccttaaagatgcagaatgtcaaaatcacaaaaccacagagctttacaaagctagtcaaaatgtcagcacctgcgaatggccgtctttaagcttctctgccagaagcctgggactttggggacagcagagccccctgggagtcagggttttcgaggctcaggagggtgggaagctcaaaatgagaggccttgtgggccaagctccagagcccagcccacagcctccataggtgccctgtccccacccacagggatcaacgtggccagcctggaatgggtgtccagggagccggcactgctctgcaccttcccaaatcccagtgcacccaggaaggacaggtcagtggacagggctgggaaggatcctcgccctcctatccgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 149 - DNA, 6707 bp, Artificial Sequence
  3105 pAAV_FOXP3.08_MND.GFPki(1staa)_08_for T9
gtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcggccgcatctcaggtaatgtcagctcggtccttccagctgctcaagctaaaacccatgtcactttgactctccctcttgcccactacatccaagctgctagcactgctcctgatccagcttcagattaagtctcagaatctacccacttctcgccttctccactgccaccagcccattctgtgccagcatcatcacttgccaggactgttacaatagcctcctcactagccccactcacagcagccagatgaatcttttgagtccatgcctagtcactggggcaaaataggactccgaggagaaagtccgagaccagctccggcaagatgagcaaacacagcctgtgcagggtgcagggagggctagaggcctgaggcttgaaacagctctcaagtggagggggaaacaaccattgccctcatagaggacacatccacaccagggctgtgctagcgtgggcaggcaagccaggtgctggacctctgcacgtggggcatgtgtgggtatgtacatgtacctgtgttcttggtgtgtgtgtgtgtgtgtgtgtgtgtgtgtgtctagagctggggtgcaactatggggcccctcgggacatgtcccagccaatgcctgctttgaccagaggagtgtccacgtggctcaggtggtcgagtatctcataccgccctagcacacgtgtgactcctttcccctattgtctacgcagcctgcccttggacaaggacccgatgcccaaccccaggcctggcaagccctcggccccttccttggcccttggcccatccccacgcgtaggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgtctacgcagcctgcccttggacaaggacccgatgcccaaccccaggcctgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagatgcccaaccccaggcctggcaagccctcggccccttccttggcccttggcccatctcctggtgcatcgcccagctggagggctgcccctaaagcaagcgacctgctgggggcccggggcccgggtggcacgtttcaaggccgagatcttcgaggcggggcccatgcctcctcttcttccttgaaccccatgccaccatcgcagctgcaggtgaggccctgggcccaggatggggcaggcagggtggggtacctggacctacaggtgccgacctttactgtggcactgggcgggaggggggctggctggggcacaggaagtggtttctgggtcccaggcaagtctgtgacttatgcagatgttgcagggccaagaaaatccccacctgccaggcctcagagattggaggctctccccgacctcccaatccctgtctcaggagaggaggaggccgtattgtagtcccatgagcatagctatgtgtccccatccccatgtgacaagagaagaggactggggccaagtaggtgaggtgacagggctgaggccagctctgcaacttattagctgtttgatctttaaaaagttactcgatctccatgagcctcagtttccatacgtgtaaaagggggatgatcatagcatctaccatgtgggcttgcagtgcagagtatttgaattagacacagaacagtgaggatcaggatggcctctcacccacctgcctttctgcccagctgcccacactgcccctagtcatggtggcaccctccggggcacggctgggccccttgccccacttacaggcactcctccaggacaggccacatttcatgcaccaggtatggacggtgaatggatcctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagacccc

SEQ ID NO: 150 - DNA, 7894 bp, Artificial Sequence
  3066 pAAV_FOXP3.06_MND.FOXP3geneartCDS.P2A.LNGFR.pA_06_for T9
gtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcggccgcatcacttgccaggactgttacaatagcctcctcactagccccactcacagcagccagatgaatcttttgagtccatgcctagtcactggggcaaaataggactccgaggagaaagtccgagaccagctccggcaagatgagcaaacacagcctgtgcagggtgcagggagggctagaggcctgaggcttgaaacagctctcaagtggagggggaaacaaccattgccctcatagaggacacatccacaccagggctgtgctagcgtgggcaggcaagccaggtgctggacctctgcacgtggggcatgtgtgggtatgtacatgtacctgtgttcttggtgtgtgtgtgtgtgtgtgtgtgtgtgtgtgtctagagctggggtgcaactatggggcccctcgggacatgtcccagccaatgcctgctttgaccagaggagtgtccacgtggctcaggtggtcgagtatctcataccgccctagcacacgtgtgactcctttcccctattgtctacgcagcctgcccttggacaaggacccgatgcccaaccccaggcctggcaagccctcggccccttccttggcccttggcccatccccacgcgtaggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggatctcgaggccaccatgcctaatcctcggcctggaaagcctagcgctccttctcttgctctgggaccttctcctggcgcctctccatcttggagagccgctcctaaagccagcgatctgctgggagctagaggacctggcggcacatttcagggcagagatcttagaggcggagcccacgctagctcctccagccttaatcctatgcctcctagccagctccagctgcctacactgcctctggttatggtggctcctagcggagctagactgggccctctgcctcatctgcaagctctgctgcaggacagaccccacttcatgcaccagctgagcaccgtggatgcccacgcaagaacacctgtgctgcaggttcaccctctggaatccccagccatgatcagcctgacacctccaacaacagccaccggcgtgttcagcctgaaagccagacctggactgcctcctggcatcaatgtggccagcctggaatgggtgtccagagaacctgctctgctgtgcacattccccaatccaagcgctcccagaaaggacagcacactgtctgccgtgcctcagagcagctatcccctgcttgctaacggcgtgtgcaagtggcctggatgcgagaaggtgttcgaggaacccgaggacttcctgaagcactgccaggccgatcatctgctggacgagaaaggcagagcccagtgtctgctccagcgcgagatggtgcagtctctggaacagcagctggtcctggaaaaagaaaagctgagcgccatgcaggcccacctggccggaaaaatggccctgacaaaggccagcagcgtggcctcttctgataagggcagctgctgcattgtggccgctggatctcagggacctgtggttcctgcttggagcggacctagagaggcccctgattctctgtttgccgtgcggagacacctgtggggctctcacggcaactctactttccccgagttcctgcacaacatggactacttcaagttccacaacatgcggcctccattcacctacgccacactgatcagatgggccattctggaagcccctgagaagcagagaaccctgaacgagatctaccactggtttacccggatgttcgccttcttccggaatcaccctgccacctggaagaacgccatccggcacaatctgagcctgcacaagtgcttcgtgcgcgtggaatctgagaaaggcgccgtgtggacagtggacgagctggaattcagaaagaagagaagccagcggcctagccggtgcagcaatcctacacctggacctggaagcggagcgactaacttcagcctgctgaagcaggccggagatgtggaggaaaaccctggaccgatgggggcaggtgccaccggacgagccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggtgaaagcttgtcgactgctttatttgtgaaatttgtgatgctattgctttatttgtaaccattataagctgcaataaacaagttaacaacaacaattgcattcattttatgtttcaggttcagggggagatgtgggaggttttttaaagcactagtgcctcgcccagctggagggctgcacccaaagcctcagacctgctgggggcccggggcccagggggaaccttccagggccgagatcttcgaggcggggcccatgcctcctcttcttccttgaaccccatgccaccatcgcagctgcaggtgaggccctgggcccaggatggggcaggcagggtggggtacctggacctacaggtgccgacctttactgtggcactgggcgggaggggggctggctggggcacaggaagtggtttctgggtcccaggcaagtctgtgacttatgcagatgttgcagggccaagaaaatccccacctgccaggcctcagagattggaggctctccccgacctcccaatccctgtctcaggagaggaggaggccgtattgtagtcccatgagcatagctatgtgtccccatccccatgtgacaagagaagaggactggggccaagtaggtgaggtgacagggctgaggccagctctgcaacttattagctgtttgatctttaaaaagttactcgatctccatgagcctcagtttccatacgtgtaaaagggggatgatcatagcatctaccatgtgggcttgcaggatcctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagacccc

SEQ ID NO: 151 - DNA, 6508 bp, Artificial Sequence
  3080 pAAV_FOXP3.06_MND.LNGFR-P2A-KI_0.6 for KI
gtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcggccgcatcacttgccaggactgttacaatagcctcctcactagccccactcacagcagccagatgaatcttttgagtccatgcctagtcactggggcaaaataggactccgaggagaaagtccgagaccagctccggcaagatgagcaaacacagcctgtgcagggtgcagggagggctagaggcctgaggcttgaaacagctctcaagtggagggggaaacaaccattgccctcatagaggacacatccacaccagggctgtgctagcgtgggcaggcaagccaggtgctggacctctgcacgtggggcatgtgtgggtatgtacatgtacctgtgttcttggtgtgtgtgtgtgtgtgtgtgtgtgtgtgtgtctagagctggggtgcaactatggggcccctcgggacatgtcccagccaatgcctgctttgaccagaggagtgtccacgtggctcaggtggtcgagtatctcataccgccctagcacacgtgtgactcctttcccctattgtctacgcagcctgcccttggacaaggacccgatgcccaaccccaggcctggcaagccctcggccccttccttggcccttggcccatccccacgcgtaggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggatctcgaggccaccatgggggcaggtgccaccggacgagccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggggaagcggagcgactaacttcagcctgctgaagcaggccggagatgtggaggaaaaccctggaccgatgcccaaccccaggcctggcaagccctcggccccttccttggcccttggcccatctcctggtgcatcgcccagctggagggctgcccctaaagcaagcgacctgctgggggcccggggcccgggtggcacgttccagggccgagatcttcgaggcggggcccatgcctcctcttcttccttgaaccccatgccaccatcgcagctgcaggtgaggccctgggcccaggatggggcaggcagggtggggtacctggacctacaggtgccgacctttactgtggcactgggcgggaggggggctggctggggcacaggaagtggtttctgggtcccaggcaagtctgtgacttatgcagatgttgcagggccaagaaaatccccacctgccaggcctcagagattggaggctctccccgacctcccaatccctgtctcaggagaggaggaggccgtattgtagtcccatgagcatagctatgtgtccccatccccatgtgacaagagaagaggactggggccaagtaggtgaggtgacagggctgaggccagctctgcaacttattagctgtttgatctttaaaaagttactcgatctccatgagcctcagtttccatacgtgtaaaagggggatgatcatagcatctaccatgtgggcttgcaggatcctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagacccc

SEQ ID NO: 152 - DNA, 210 bp, Artificial Sequence
  Human FOXP3 1st coding exon sequences included in AAV 3080 (modified to be non-cleavable by TALEN, Cas9/T3 or Cas9/T4 or Cas9/T9)
atgcccaaccccaggcctggcaagccctcggccccttccttggcccttggcccatctcctggtgcatcgcccagctggagggctgcccctaaagcaagcgacctgctgggggcccggggcccgggtggcacgttccagggccgagatcttcgaggcggggcccatgcctcctcttcttccttgaaccccatgccaccatcgcagctgcag

SEQ ID NO: 153 - DNA, 7894 bp, Artificial Sequence
  3098 pAAV_FOXP3.06_MND.FOXP3geneartCDS.R397W.P2A.LNGFR.pA_06_for T9
gtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcggccgcatcacttgccaggactgttacaatagcctcctcactagccccactcacagcagccagatgaatcttttgagtccatgcctagtcactggggcaaaataggactccgaggagaaagtccgagaccagctccggcaagatgagcaaacacagcctgtgcagggtgcagggagggctagaggcctgaggcttgaaacagctctcaagtggagggggaaacaaccattgccctcatagaggacacatccacaccagggctgtgctagcgtgggcaggcaagccaggtgctggacctctgcacgtggggcatgtgtgggtatgtacatgtacctgtgttcttggtgtgtgtgtgtgtgtgtgtgtgtgtgtgtgtctagagctggggtgcaactatggggcccctcgggacatgtcccagccaatgcctgctttgaccagaggagtgtccacgtggctcaggtggtcgagtatctcataccgccctagcacacgtgtgactcctttcccctattgtctacgcagcctgcccttggacaaggacccgatgcccaaccccaggcctggcaagccctcggccccttccttggcccttggcccatccccacgcgtaggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggatctcgaggccaccatgcctaatcctcggcctggaaagcctagcgctccttctcttgctctgggaccttctcctggcgcctctccatcttggagagccgctcctaaagccagcgatctgctgggagctagaggacctggcggcacatttcagggcagagatcttagaggcggagcccacgctagctcctccagccttaatcctatgcctcctagccagctccagctgcctacactgcctctggttatggtggctcctagcggagctagactgggccctctgcctcatctgcaagctctgctgcaggacagaccccacttcatgcaccagctgagcaccgtggatgcccacgcaagaacacctgtgctgcaggttcaccctctggaatccccagccatgatcagcctgacacctccaacaacagccaccggcgtgttcagcctgaaagccagacctggactgcctcctggcatcaatgtggccagcctggaatgggtgtccagagaacctgctctgctgtgcacattccccaatccaagcgctcccagaaaggacagcacactgtctgccgtgcctcagagcagctatcccctgcttgctaacggcgtgtgcaagtggcctggatgcgagaaggtgttcgaggaacccgaggacttcctgaagcactgccaggccgatcatctgctggacgagaaaggcagagcccagtgtctgctccagcgcgagatggtgcagtctctggaacagcagctggtcctggaaaaagaaaagctgagcgccatgcaggcccacctggccggaaaaatggccctgacaaaggccagcagcgtggcctcttctgataagggcagctgctgcattgtggccgctggatctcagggacctgtggttcctgcttggagcggacctagagaggcccctgattctctgtttgccgtgcggagacacctgtggggctctcacggcaactctactttccccgagttcctgcacaacatggactacttcaagttccacaacatgcggcctccattcacctacgccacactgatcagatgggccattctggaagcccctgagaagcagagaaccctgaacgagatctaccactggtttacccggatgttcgccttcttccggaatcaccctgccacctggaagaacgccatccggcacaatctgagcctgcacaagtgcttcgtgtgggtggaatctgagaaaggcgccgtgtggacagtggacgagctggaattcagaaagaagagaagccagcggcctagccggtgcagcaatcctacacctggacctggaagcggagcgactaacttcagcctgctgaagcaggccggagatgtggaggaaaaccctggaccgatgggggcaggtgccaccggacgagccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggtgaaagcttgtcgactgctttatttgtgaaatttgtgatgctattgctttatttgtaaccattataagctgcaataaacaagttaacaacaacaattgcattcattttatgtttcaggttcagggggagatgtgggaggttttttaaagcactagtgcctcgcccagctggagggctgcacccaaagcctcagacctgctgggggcccggggcccagggggaaccttccagggccgagatcttcgaggcggggcccatgcctcctcttcttccttgaaccccatgccaccatcgcagctgcaggtgaggccctgggcccaggatggggcaggcagggtggggtacctggacctacaggtgccgacctttactgtggcactgggcgggaggggggctggctggggcacaggaagtggtttctgggtcccaggcaagtctgtgacttatgcagatgttgcagggccaagaaaatccccacctgccaggcctcagagattggaggctctccccgacctcccaatccctgtctcaggagaggaggaggccgtattgtagtcccatgagcatagctatgtgtccccatccccatgtgacaagagaagaggactggggccaagtaggtgaggtgacagggctgaggccagctctgcaacttattagctgtttgatctttaaaaagttactcgatctccatgagcctcagtttccatacgtgtaaaagggggatgatcatagcatctaccatgtgggcttgcaggatcctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagacccc

SEQ ID NO: 154 - DNA, 7643 bp, Artificial Sequence
  3132_pAAV_FOXP3.06_MND.FOXP3geneartCDS.P2A.LNGFR.pA_06_for T9.kanamycin a.k.a.3066kanamycin
gtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcggccgcatcacttgccaggactgttacaatagcctcctcactagccccactcacagcagccagatgaatcttttgagtccatgcctagtcactggggcaaaataggactccgaggagaaagtccgagaccagctccggcaagatgagcaaacacagcctgtgcagggtgcagggagggctagaggcctgaggcttgaaacagctctcaagtggagggggaaacaaccattgccctcatagaggacacatccacaccagggctgtgctagcgtgggcaggcaagccaggtgctggacctctgcacgtggggcatgtgtgggtatgtacatgtacctgtgttcttggtgtgtgtgtgtgtgtgtgtgtgtgtgtgtgtctagagctggggtgcaactatggggcccctcgggacatgtcccagccaatgcctgctttgaccagaggagtgtccacgtggctcaggtggtcgagtatctcataccgccctagcacacgtgtgactcctttcccctattgtctacgcagcctgcccttggacaaggacccgatgcccaaccccaggcctggcaagccctcggccccttccttggcccttggcccatccccacgcgtaggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggatctcgaggccaccatgcctaatcctcggcctggaaagcctagcgctccttctcttgctctgggaccttctcctggcgcctctccatcttggagagccgctcctaaagccagcgatctgctgggagctagaggacctggcggcacatttcagggcagagatcttagaggcggagcccacgctagctcctccagccttaatcctatgcctcctagccagctccagctgcctacactgcctctggttatggtggctcctagcggagctagactgggccctctgcctcatctgcaagctctgctgcaggacagaccccacttcatgcaccagctgagcaccgtggatgcccacgcaagaacacctgtgctgcaggttcaccctctggaatccccagccatgatcagcctgacacctccaacaacagccaccggcgtgttcagcctgaaagccagacctggactgcctcctggcatcaatgtggccagcctggaatgggtgtccagagaacctgctctgctgtgcacattccccaatccaagcgctcccagaaaggacagcacactgtctgccgtgcctcagagcagctatcccctgcttgctaacggcgtgtgcaagtggcctggatgcgagaaggtgttcgaggaacccgaggacttcctgaagcactgccaggccgatcatctgctggacgagaaaggcagagcccagtgtctgctccagcgcgagatggtgcagtctctggaacagcagctggtcctggaaaaagaaaagctgagcgccatgcaggcccacctggccggaaaaatggccctgacaaaggccagcagcgtggcctcttctgataagggcagctgctgcattgtggccgctggatctcagggacctgtggttcctgcttggagcggacctagagaggcccctgattctctgtttgccgtgcggagacacctgtggggctctcacggcaactctactttccccgagttcctgcacaacatggactacttcaagttccacaacatgcggcctccattcacctacgccacactgatcagatgggccattctggaagcccctgagaagcagagaaccctgaacgagatctaccactggtttacccggatgttcgccttcttccggaatcaccctgccacctggaagaacgccatccggcacaatctgagcctgcacaagtgcttcgtgcgcgtggaatctgagaaaggcgccgtgtggacagtggacgagctggaattcagaaagaagagaagccagcggcctagccggtgcagcaatcctacacctggacctggaagcggagcgactaacttcagcctgctgaagcaggccggagatgtggaggaaaaccctggaccgatgggggcaggtgccaccggacgagccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggtgaaagcttgtcgactgctttatttgtgaaatttgtgatgctattgctttatttgtaaccattataagctgcaataaacaagttaacaacaacaattgcattcattttatgtttcaggttcagggggagatgtgggaggttttttaaagcactagtgcctcgcccagctggagggctgcacccaaagcctcagacctgctgggggcccggggcccagggggaaccttccagggccgagatcttcgaggcggggcccatgcctcctcttcttccttgaaccccatgccaccatcgcagctgcaggtgaggccctgggcccaggatggggcaggcagggtggggtacctggacctacaggtgccgacctttactgtggcactgggcgggaggggggctggctggggcacaggaagtggtttctgggtcccaggcaagtctgtgacttatgcagatgttgcagggccaagaaaatccccacctgccaggcctcagagattggaggctctccccgacctcccaatccctgtctcaggagaggaggaggccgtattgtagtcccatgagcatagctatgtgtccccatccccatgtgacaagagaagaggactggggccaagtaggtgaggtgacagggctgaggccagctctgcaacttattagctgtttgatctttaaaaagttactcgatctccatgagcctcagtttccatacgtgtaaaagggggatgatcatagcatctaccatgtgggcttgcaggatcctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtctacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatctcatgaccaaaatcccttaacgtgagttttcgttccactgagcgtcagacccc

SEQ ID NO: 155 - DNA, 7596 bp, Artificial Sequence
  3117 pAAV_FOXP3.045_MND.LNGFR-P2A-FOXP3geneartCDS.pA_045_for T9
gtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcggccgcagcctgtgcagggtgcagggagggctagaggcctgaggcttgaaacagctctcaagtggagggggaaacaaccattgccctcatagaggacacatccacaccagggctgtgctagcgtgggcaggcaagccaggtgctggacctctgcacgtggggcatgtgtgggtatgtacatgtacctgtgttcttggtgtgtgtgtgtgtgtgtgtgtgtgtgtgtgtctagagctggggtgcaactatggggcccctcgggacatgtcccagccaatgcctgctttgaccagaggagtgtccacgtggctcaggtggtcgagtatctcataccgccctagcacacgtgtgactcctttcccctattgtctacgcagcctgcccttggacaaggacccgatgcccaaccccaggcctggcaagccctcggccccttccttggcccttggcccatccccacgcgtaggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggatctcgaggccaccatgggggcaggtgccaccggacgagccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggggaagcggagcgactaacttcagcctgctgaagcaggccggagatgtggaggaaaaccctggaccgatgcctaatcctcggcctggaaagcctagcgctccttctcttgctctgggaccttctcctggcgcctctccatcttggagagccgctcctaaagccagcgatctgctgggagctagaggacctggcggcacatttcagggcagagatcttagaggcggagcccacgctagctcctccagccttaatcctatgcctcctagccagctccagctgcctacactgcctctggttatggtggctcctagcggagctagactgggccctctgcctcatctgcaagctctgctgcaggacagaccccacttcatgcaccagctgagcaccgtggatgcccacgcaagaacacctgtgctgcaggttcaccctctggaatccccagccatgatcagcctgacacctccaacaacagccaccggcgtgttcagcctgaaagccagacctggactgcctcctggcatcaatgtggccagcctggaatgggtgtccagagaacctgctctgctgtgcacattccccaatccaagcgctcccagaaaggacagcacactgtctgccgtgcctcagagcagctatcccctgcttgctaacggcgtgtgcaagtggcctggatgcgagaaggtgttcgaggaacccgaggacttcctgaagcactgccaggccgatcatctgctggacgagaaaggcagagcccagtgtctgctccagcgcgagatggtgcagtctctggaacagcagctggtcctggaaaaagaaaagctgagcgccatgcaggcccacctggccggaaaaatggccctgacaaaggccagcagcgtggcctcttctgataagggcagctgctgcattgtggccgctggatctcagggacctgtggttcctgcttggagcggacctagagaggcccctgattctctgtttgccgtgcggagacacctgtggggctctcacggcaactctactttccccgagttcctgcacaacatggactacttcaagttccacaacatgcggcctccattcacctacgccacactgatcagatgggccattctggaagcccctgagaagcagagaaccctgaacgagatctaccactggtttacccggatgttcgccttcttccggaatcaccctgccacctggaagaacgccatccggcacaatctgagcctgcacaagtgcttcgtgcgcgtggaatctgagaaaggcgccgtgtggacagtggacgagctggaattcagaaagaagagaagccagcggcctagccggtgcagcaatcctacacctggaccttgaaagcttgtcgactgctttatttgtgaaatttgtgatgctattgctttatttgtaaccattataagctgcaataaacaagttaacaacaacaattgcattcattttatgtttcaggttcagggggagatgtgggaggttttttaaagcactagtgcctcgcccagctggagggctgcacccaaagcctcagacctgctgggggcccggggcccagggggaaccttccagggccgagatcttcgaggcggggcccatgcctcctcttcttccttgaaccccatgccaccatcgcagctgcaggtgaggccctgggcccaggatggggcaggcagggtggggtacctggacctacaggtgccgacctttactgtggcactgggcgggaggggggctggctggggcacaggaagtggtttctgggtcccaggcaagtctgtgacttatgcagatgttgcagggccaagaaaatccccacctgccaggcctcagagattggaggctctccccgacctcccaatccctgtctcaggagaggaggaggccgtattgtagtcccatgagcatagctatgtgtccccatccccatgtgacaagagaagaggaggatcctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagacccc

SEQ ID NO: 156 - DNA, 8359 bp, Artificial Sequence
  3118 pAAV_FOXP3.045_MND.LNGFR-P2A-FOXP3geneartCDS.3UTR_045_for T9
gtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcggccgcagcctgtgcagggtgcagggagggctagaggcctgaggcttgaaacagctctcaagtggagggggaaacaaccattgccctcatagaggacacatccacaccagggctgtgctagcgtgggcaggcaagccaggtgctggacctctgcacgtggggcatgtgtgggtatgtacatgtacctgtgttcttggtgtgtgtgtgtgtgtgtgtgtgtgtgtgtgtctagagctggggtgcaactatggggcccctcgggacatgtcccagccaatgcctgctttgaccagaggagtgtccacgtggctcaggtggtcgagtatctcataccgccctagcacacgtgtgactcctttcccctattgtctacgcagcctgcccttggacaaggacccgatgcccaaccccaggcctggcaagccctcggccccttccttggcccttggcccatccccacgcgtaggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggatctcgaggccaccatgggggcaggtgccaccggacgagccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggggaagcggagcgactaacttcagcctgctgaagcaggccggagatgtggaggaaaaccctggaccgatgcctaatcctcggcctggaaagcctagcgctccttctcttgctctgggaccttctcctggcgcctctccatcttggagagccgctcctaaagccagcgatctgctgggagctagaggacctggcggcacatttcagggcagagatcttagaggcggagcccacgctagctcctccagccttaatcctatgcctcctagccagctccagctgcctacactgcctctggttatggtggctcctagcggagctagactgggccctctgcctcatctgcaagctctgctgcaggacagaccccacttcatgcaccagctgagcaccgtggatgcccacgcaagaacacctgtgctgcaggttcaccctctggaatccccagccatgatcagcctgacacctccaacaacagccaccggcgtgttcagcctgaaagccagacctggactgcctcctggcatcaatgtggccagcctggaatgggtgtccagagaacctgctctgctgtgcacattccccaatccaagcgctcccagaaaggacagcacactgtctgccgtgcctcagagcagctatcccctgcttgctaacggcgtgtgcaagtggcctggatgcgagaaggtgttcgaggaacccgaggacttcctgaagcactgccaggccgatcatctgctggacgagaaaggcagagcccagtgtctgctccagcgcgagatggtgcagtctctggaacagcagctggtcctggaaaaagaaaagctgagcgccatgcaggcccacctggccggaaaaatggccctgacaaaggccagcagcgtggcctcttctgataagggcagctgctgcattgtggccgctggatctcagggacctgtggttcctgcttggagcggacctagagaggcccctgattctctgtttgccgtgcggagacacctgtggggctctcacggcaactctactttccccgagttcctgcacaacatggactacttcaagttccacaacatgcggcctccattcacctacgccacactgatcagatgggccattctggaagcccctgagaagcagagaaccctgaacgagatctaccactggtttacccggatgttcgccttcttccggaatcaccctgccacctggaagaacgccatccggcacaatctgagcctgcacaagtgcttcgtgcgcgtggaatctgagaaaggcgccgtgtggacagtggacgagctggaattcagaaagaagagaagccagcggcctagccggtgcagcaatcctacacctggaccttgaaagcttgtcgaccctcaagatcaaggaaaggaggatggacgaacaggggccaaactggtgggaggcagaggtggtgggggcagggatgataggccctggatgtgcccacagggaccaagaagtgaggtttccactgtcttgcctgccagggcccctgttcccccgctggcagccaccccctcccccatcatatcctttgccccaaggctgctcagaggggccccggtcctggccccagcccccacctccgccccagacacaccccccagtcgagccctgcagccaaacagagccttcacaaccagccacacagagcctgcctcagctgctcgcacagattacttcagggctggaaaagtcacacagacacacaaaatgtcacaatcctgtccctcactcaacacaaaccccaaaacacagagagcctgcctcagtacactcaaacaacctcaaagctgcatcatcacacaatcacacacaagcacagccctgacaacccacacaccccaaggcacgcacccacagccagcctcagggcccacaggggcactgtcaacacaggggtgtgcccagaggcctacacagaagcagcgtcagtaccctcaggatctgaggtcccaacacgtgctcgctcacacacacggcctgttagaattcacctgtgtatctcacgcatatgcacacgcacagccccccagtgggtctcttgagtcccgtgcagacacacacagccacacacactgccttgccaaaaataccccgtgtctcccctgccactcacctcactcccattccctgagccctgatccatgcctcagcttagactgcagaggaactactcatttatttgggatccaaggcccccaacccacagtaccgtccccaataaactgcagccgagctccccacaactagtgcctcgcccagctggagggctgcacccaaagcctcagacctgctgggggcccggggcccagggggaaccttccagggccgagatcttcgaggcggggcccatgcctcctcttcttccttgaaccccatgccaccatcgcagctgcaggtgaggccctgggcccaggatggggcaggcagggtggggtacctggacctacaggtgccgacctttactgtggcactgggcgggaggggggctggctggggcacaggaagtggtttctgggtcccaggcaagtctgtgacttatgcagatgttgcagggccaagaaaatccccacctgccaggcctcagagattggaggctctccccgacctcccaatccctgtctcaggagaggaggaggccgtattgtagtcccatgagcatagctatgtgtccccatccccatgtgacaagagaagaggaggatcctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagacccc

SEQ ID NO: 157 - DNA, 6889 bp, Artificial Sequence
  1390 pAAV-FOXP3_0.9[MND-GFPki]_0.9 (noUCOEctrl)
gtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcggccgcgctcaagagaccccatctctcctcctctctgtcacttgccatgctggatccgtgcatgatcacactcctggactcgcctccttgccctgagatccagacccccgtattcagctgccccctcagctcctccactcacatatttaatgccagactcttcatgtctatctacacctgcacttttgcacccaatccaactccccgccatgtcccccatctcaggtaatgtcagctcggtccttccagctgctcaagctaaaacccatgtcactttgactctccctcttgcccactacatccaagctgctagcactgctcctgatccagcttcagattaagtctcagaatctacccacttctcgccttctccactgccaccagcccattctgtgccagcatcatcacttgccaggactgttacaatagcctcctcactagccccactcacagcagccagatgaatcttttgagtccatgcctagtcactggggcaaaataggactccgaggagaaagtccgagaccagctccggcaagatgagcaaacacagcctgtgcagggtgcagggagggctagaggcctgaggcttgaaacagctctcaagtggagggggaaacaaccattgccctcatagaggacacatccacaccagggctgtgctagcgtgggcaggcaagccaggtgctggacctctgcacgtggggcatgtgtgggtatgtacatgtacctgtgttcttggtgtgtgtgtgtgtgtgtgtgtgtgtgtgtgtctagagctggggtgcaactatggggcccctcgggacatgtcccagccaatgcctgctttgaccagaggagtgtccacgtggctcaggtggtcgagtatctcataccgccctagcacacgtgtgactcctttcccctattgtctaccccggggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgtctacgcagcctgcccttggacaaggacccgatgcccaaccccaggcctgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggcaagccctcggccccttccttggcccttggcccatccccaggagcctcgcccagctggagggctgcccctaaagcaagcgacctgctgggggcccggggcccgggtggcacgttccagggccgagatcttcgaggcggggcccatgcctcctcttcttccttgaaccccatgccaccatcgcagctgcaggtgaggccctgggcccaggatggggcaggcagggtggggtacctggacctacaggtgccgacctttactgtggcactgggcgggaggggggctggctggggcacaggaagtggtttctgggtcccaggcaagtctgtgacttatgcagatgttgcagggccaagaaaatccccacctgccaggcctcagagattggaggctctccccgacctcccaatccctgtctcaggagaggaggaggccgtattgtagtcccatgagcatagctatgtgtccccatccccatgtgacaagagaagaggactggggccaagtaggtgaggtgacagggctgaggccagctctgcaacttattagctgtttgatctttaaaaagttactcgatctccatgagcctcagtttccatacgtgtaaaagggggatgatcatagcatctaccatgtgggcttgcagtgcagagtatttgaattagacacagaacagtgaggatcaggatggcctctcacccacctgcctttctgcccagctgcccacactgcccctagtcatggtggcaccctccggggcacggctgggccccttgccccacttacaggcactcctccaggacaggccacatttcatgcaccaggtatggacggtgaatgggcagggaggagggagcaggtgggagaactgtggggaggggccccgagtcaggctgaaccacagcccacatggcggccgctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagacccc

SEQ ID NO: 158 - DNA, 7552 bp, Artificial Sequence
  1391 pAAV-FOXP3_0.9[FWD0.7UCOE-MND-GFPki]_0.9
gtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcggccgcgctcaagagaccccatctctcctcctctctgtcacttgccatgctggatccgtgcatgatcacactcctggactcgcctccttgccctgagatccagacccccgtattcagctgccccctcagctcctccactcacatatttaatgccagactcttcatgtctatctacacctgcacttttgcacccaatccaactccccgccatgtcccccatctcaggtaatgtcagctcggtccttccagctgctcaagctaaaacccatgtcactttgactctccctcttgcccactacatccaagctgctagcactgctcctgatccagcttcagattaagtctcagaatctacccacttctcgccttctccactgccaccagcccattctgtgccagcatcatcacttgccaggactgttacaatagcctcctcactagccccactcacagcagccagatgaatcttttgagtccatgcctagtcactggggcaaaataggactccgaggagaaagtccgagaccagctccggcaagatgagcaaacacagcctgtgcagggtgcagggagggctagaggcctgaggcttgaaacagctctcaagtggagggggaaacaaccattgccctcatagaggacacatccacaccagggctgtgctagcgtgggcaggcaagccaggtgctggacctctgcacgtggggcatgtgtgggtatgtacatgtacctgtgttcttggtgtgtgtgtgtgtgtgtgtgtgtgtgtgtgtctagagctggggtgcaactatggggcccctcgggacatgtcccagccaatgcctgctttgaccagaggagtgtccacgtggctcaggtggtcgagtatctcataccgccctagcacacgtgtgactcctttcccctattgtctacgcaaacacccgaatcaacttctagtcaaattattgttcacgccgcaatgacccacccctggcccgcgtctgtggaactgacccctggtgtacaggagagttcgctgctgaaagtggtcccaaaggggtactagtttttaagctcccaactccccctcccccagcgtctggaggattccacaccctcgcaccgcaggggcgaggaagtgggcggagtccggttttggcgccagccgctgaggctgccaagcagaaaagccaccgctgaggagactccggtcactgtcctcgccccgcctcccccttccctccccttggggaccaccgggcgccacgccgcgaacggtaagtgccgcggtcgtcggcgcctccgccctccccctagggccccaattcccagcgggcgcggcgcgcggcccctccccccgccgggcgcgcgcccgctgccccgcccttcgtggccgcccggcgtgggcggtgccacccctccccccggcggccccgcgcgcagctcccggctccctcccccttcggatgtggcttgagctgtaggcgcggagggccggagacgctgcagacccgcgacccggagcagctcggaggcggtgaagtcggtggctttccttctctctagctctcgctcgctggtggtgcttcagatgccacacgcgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgtctacgcagcctgcccttggacaaggacccgatgcccaaccccaggcctgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggcaagccctcggccccttccttggcccttggcccatccccaggagcctcgcccagctggagggctgcccctaaagcaagcgacctgctgggggcccggggcccgggtggcacgttccagggccgagatcttcgaggcggggcccatgcctcctcttcttccttgaaccccatgccaccatcgcagctgcaggtgaggccctgggcccaggatggggcaggcagggtggggtacctggacctacaggtgccgacctttactgtggcactgggcgggaggggggctggctggggcacaggaagtggtttctgggtcccaggcaagtctgtgacttatgcagatgttgcagggccaagaaaatccccacctgccaggcctcagagattggaggctctccccgacctcccaatccctgtctcaggagaggaggaggccgtattgtagtcccatgagcatagctatgtgtccccatccccatgtgacaagagaagaggactggggccaagtaggtgaggtgacagggctgaggccagctctgcaacttattagctgtttgatctttaaaaagttactcgatctccatgagcctcagtttccatacgtgtaaaagggggatgatcatagcatctaccatgtgggcttgcagtgcagagtatttgaattagacacagaacagtgaggatcaggatggcctctcacccacctgcctttctgcccagctgcccacactgcccctagtcatggtggcaccctccggggcacggctgggccccttgccccacttacaggcactcctccaggacaggccacatttcatgcaccaggtatggacggtgaatgggcagggaggagggagcaggtgggagaactgtggggaggggccccgagtcaggctgaaccacagcccacatggcggccgctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagacccc

SEQ ID NO: 159 - DNA, 7558 bp, Artificial Sequence
  1392 pAAV-FOXP3_0.9[RVS0.7UCOE-MND-GFPki]_0.9
gtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcggccgcgctcaagagaccccatctctcctcctctctgtcacttgccatgctggatccgtgcatgatcacactcctggactcgcctccttgccctgagatccagacccccgtattcagctgccccctcagctcctccactcacatatttaatgccagactcttcatgtctatctacacctgcacttttgcacccaatccaactccccgccatgtcccccatctcaggtaatgtcagctcggtccttccagctgctcaagctaaaacccatgtcactttgactctccctcttgcccactacatccaagctgctagcactgctcctgatccagcttcagattaagtctcagaatctacccacttctcgccttctccactgccaccagcccattctgtgccagcatcatcacttgccaggactgttacaatagcctcctcactagccccactcacagcagccagatgaatcttttgagtccatgcctagtcactggggcaaaataggactccgaggagaaagtccgagaccagctccggcaagatgagcaaacacagcctgtgcagggtgcagggagggctagaggcctgaggcttgaaacagctctcaagtggagggggaaacaaccattgccctcatagaggacacatccacaccagggctgtgctagcgtgggcaggcaagccaggtgctggacctctgcacgtggggcatgtgtgggtatgtacatgtacctgtgttcttggtgtgtgtgtgtgtgtgtgtgtgtgtgtgtgtctagagctggggtgcaactatggggcccctcgggacatgtcccagccaatgcctgctttgaccagaggagtgtccacgtggctcaggtggtcgagtatctcataccgccctagcacacgtgtgactcctttcccctattgtctacccgggtgtggcatctgaagcaccaccagcgagcgagagctagagagaaggaaagccaccgacttcaccgcctccgagctgctccgggtcgcgggtctgcagcgtctccggccctccgcgcctacagctcaagccacatccgaagggggagggagccgggagctgcgcgcggggccgccggggggaggggtggcaccgcccacgccgggcggccacgaagggcggggcagcgggcgcgcgcccggcggggggaggggccgcgcgccgcgcccgctgggaattggggccctagggggagggcggaggcgccgacgaccgcggcacttaccgttcgcggcgtggcgcccggtggtccccaaggggagggaagggggaggcggggcgaggacagtgaccggagtctcctcagcggtggcttttctgcttggcagcctcagcggctggcgccaaaaccggactccgcccacttcctcgcccctgcggtgcgagggtgtggaatcctccagacgctgggggagggggagttgggagcttaaaaactagtacccctttgggaccactttcagcagcgaactctcctgtacaccaggggtcagttccacagacgcgggccaggggtgggtcattgcggcgtgaacaataatttgactagaagttgattcgggtgtttcccggggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgtctacgcagcctgcccttggacaaggacccgatgcccaaccccaggcctgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggcaagccctcggccccttccttggcccttggcccatccccaggagcctcgcccagctggagggctgcccctaaagcaagcgacctgctgggggcccggggcccgggtggcacgttccagggccgagatcttcgaggcggggcccatgcctcctcttcttccttgaaccccatgccaccatcgcagctgcaggtgaggccctgggcccaggatggggcaggcagggtggggtacctggacctacaggtgccgacctttactgtggcactgggcgggaggggggctggctggggcacaggaagtggtttctgggtcccaggcaagtctgtgacttatgcagatgttgcagggccaagaaaatccccacctgccaggcctcagagattggaggctctccccgacctcccaatccctgtctcaggagaggaggaggccgtattgtagtcccatgagcatagctatgtgtccccatccccatgtgacaagagaagaggactggggccaagtaggtgaggtgacagggctgaggccagctctgcaacttattagctgtttgatctttaaaaagttactcgatctccatgagcctcagtttccatacgtgtaaaagggggatgatcatagcatctaccatgtgggcttgcagtgcagagtatttgaattagacacagaacagtgaggatcaggatggcctctcacccacctgcctttctgcccagctgcccacactgcccctagtcatggtggcaccctccggggcacggctgggccccttgccccacttacaggcactcctccaggacaggccacatttcatgcaccaggtatggacggtgaatgggcagggaggagggagcaggtgggagaactgtggggaggggccccgagtcaggctgaaccacagcccacatggcggccgctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagacccc

SEQ ID NO: 160 - DNA, 7293 bp, Artificial Sequence
  1331-pAAV-mFOXP3-MND-GFP-ki
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacagtataggatcctgaaaaacgaaagccacacttttaagggactgtaaggtagtgaggctcagcacagggacctgggtcaccatgtagagctttgaagaggaaatcagaagactgcagtatggctaagggaagaagtggacttccaagcttggcagagattggagctagtttgaggagcgcccagggaccctcaatcaagcaaccctatccctctttttttcctggcacctgccacgccaattccaagacagaagaaagcttagagaagacagacccatgctgtggccctgagctctgcagtactgaattcagctgcaagtcttccctgcctctactgcttacctttgcatttagccacatctgactatcactgtatactctgctcctccatcctctaccctccatctccagtaatgctcctgttgtagctgcttctgccaaaaacctagacatcatcttgaccctttctctcatctcctccatccaagctcccggcaacttctcctgactctgccttcagacgagacttggaagacagtcacatctcagcagctcctctgccgttatccaggttggtagcagcaacaccactcgcctcactattgcagtacacttcccactagcacagttccctggagccttcctgctcacagcatccaactgaatcttgtgaggctatgcccaagtcattggaataaaaagatgagaagagagtccaagacaagccccagtagaatcagcaaagactatgtggcctgcacagagtgcagggggtactggagggtcccacaaaccaactccccatcaccccacattcacgacagagtggtatggtgtatgtaagcaagtgaggtgctggacatgtgcatgtgtagaatatatccatcaatctgtgttcctgctgtcagggtagcatatatgtatgtaagacagaccagaggtgtagttatgaggctatcttgcaccacccctggaatgcatgtgactccattccactgttacgcgtgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgaattcatccctgcagcctgcctctgacaagaacccaatgcccaaccctaggccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggccaagcctatggctccttcgctcgcgttagggcctagcccaggagtcttgccttcgtggaaaacagcacccaagggctcagaacttctagggaccaggggctctgggggacccttccaaggtcgggacctgcgaagtggggcccacacctcttcttccttgaaccccctgccaccatcccagctgcaggtgaggcccggggcccagaatggggtaagcagggtggggtacttgggcctataggtgtcgacctttactgtggcatgtggcgggggggggggggggggctggggcacaggaagtggtttatgggtcccaggcaagtctgacttatgcagatattgcagggccaagaaaatccccactctccaggcttcagagattcaaggctttccccacccctcccaatcctcatcccgataggagaccttatgattccatggacatagccatgtatcctcatcccactgtgacgagatggctggggcccaagaaggtaacagtgttggggccagctctaccccttgaaactgttggaccttgatacattcactctccacgagcctcagattccactgatgtgaactggatagttccattgttgctaccgtgtgagactttagtaaagagctaatgaatgagacacagaactattaagatgaggctcatggcatctcatggcatctcccttctctctccagctgcctacagtgcccctagtcatggtggcaccgtctggggcccgactaggtccctcaccccacctacaggcccttctccaggacagaccacacttcatgcatcaggtatggaatcggagcaggctgggaggagggaacaaagaggacagctgtggagcagagccccaagccccgctgagccatggtccatgtgttccccagctctccactgtggatgcccatgcccagacccctgtgctccaagtgcgtccactggacaacccagccatgatcagcctcccaccaccttctgctgccactggggtcttctccctcaaggcccggcctggcctgccacctggtaacaccttcacagtatctccaagttctctaatctttgagcatgtgcaatgtaaacttttctgaattatagccctatggaggtatagaagggtcttaagagtcacggaaactccaacctccaaaaaaaaaaatatcagacttagaaccttgaagacatagaatgcaaaaaaaaccacaaatcgctattatcagtcaaaatgccgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 161 - DNA, 6408 bp, Artificial Sequence
  3209 pAAV_mFOXP3.06_PGK.GFPki_06 for mT23_from 3171
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcggccgcctcccggcaacttctcctgactctgccttcagacgagacttggaagacagtcacatctcagcagctcctctgccgttatccaggttggtagcagcaacaccactcgcctcactattgcagtacacttcccactagcacagttccctggagccttcctgctcacagcatccaactgaatcttgtgaggctatgcccaagtcattggaataaaaagatgagaagagagtccaagacaagccccagtagaatcagcaaagactatgtggcctgcacagagtgcagggggtactggagggtcccacaaaccaactccccatcaccccacattcacgacagagtggtatggtgtatgtaagcaagtgaggtgctggacatgtgcatgtgtagaatatatccatcaatctgtgttcctgctgtcagggtagcatatatgtatgtaagacagaccagaggtgtagttatgaggctatcttgcaccacccctggaatgcatgtgactccattccactgttatccctgcagcctgcctctgacaagaacccaatgcccaaccctaggccagccaagcctatggctccttccttggcccttggcccatccccagacgcgtccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcggggcgcgccgagagcagcggccgggaaggggcggtgcgggaggcggggtgtggggcggtagtgtgggccctgttcctgcccgcgcggtgaattcatccctgcagcctgcctctgacaagaacccaatgcccaaccctaggccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggccaagcctatggctccttcgctcgcgttagggcctagcccaggagtcttgccttcgtggaaaacagcacccaagggctcagaacttctagggaccaggggctctgggggacccttccaaggtcgggacctgcgaagtggggcccacacctcttcttccttgaaccccctgccaccatcccagctgcaggtgaggcccggggcccagaatggggtaagcagggtggggtacttgggcctataggtgtcgacctttactgtggcatgtggcgggggggggggggggggctggggcacaggaagtggtttatgggtcccaggcaagtctgacttatgcagatattgcagggccaagaaaatccccactctccaggcttcagagattcaaggctttccccacccctcccaatcctcatcccgataggagaccttatgattccatggacatagccatgtatcctcatcccactgtgacgagatggctggggcccaagaaggtaacagtgttggggccagctctaccccttgaaactgttggaccttgatacattcactctccacgagcctcagattccactgatgtgaactggatagttccattgttgctaccgtgtgagactttagtaaagagctaatgaatgagacacaggctagctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 162 - DNA, 7055 bp, Artificial Sequence
  3213 pAAV_mFOXP3.06_07UCOErvs.MND.GFPki_06 from 3171
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcggccgcctcccggcaacttctcctgactctgccttcagacgagacttggaagacagtcacatctcagcagctcctctgccgttatccaggttggtagcagcaacaccactcgcctcactattgcagtacacttcccactagcacagttccctggagccttcctgctcacagcatccaactgaatcttgtgaggctatgcccaagtcattggaataaaaagatgagaagagagtccaagacaagccccagtagaatcagcaaagactatgtggcctgcacagagtgcagggggtactggagggtcccacaaaccaactccccatcaccccacattcacgacagagtggtatggtgtatgtaagcaagtgaggtgctggacatgtgcatgtgtagaatatatccatcaatctgtgttcctgctgtcagggtagcatatatgtatgtaagacagaccagaggtgtagttatgaggctatcttgcaccacccctggaatgcatgtgactccattccactgttatccctgcagcctgcctctgacaagaacccaatgcccaaccctaggccagccaagcctatggctccttccttggcccttggcccatccccagacgcgtatcgatcacgagactagcctcgaaattcgagctagtcccggccgcgtgtggcatctgaagcaccaccagcgagcgagagctagagagaaggaaagccaccgacttcaccgcctccgagctgctccgggtcgcgggtctgcagcgtctccggccctccgcgcctacagctcaagccacatccgaagggggagggagccgggagctgcgcgcggggccgctggggggaggggtggcaccgcccacgccgggcggccacgaagggcggggcagcgggcgcgcgcccggcggggggaggggccgcgcgccgcgcccgctgggaattggggccctagggggagggcggaggcgccgacgaccgcggcacttaccgttcgcggcgtggcgcccggtggtccccaaggggagggaagggggaggcggggcgaggacagtgaccggagtctcctcagcggtggcttttctgcttggcagcctcagcggctggcgccaaaaccggactccgcccacttcctcgcccctgcggtgcgagggtgtggaatcctccagacgctgggggagggggagttgggagcttaaaaactagtacccctttgggaccactttcagcagcgaactctcctgtacaccaggggtcagttccacagacgcgggccaggggtgggtcattgcggcgtgaacaataatttgactagaagttgattcgggtgtttgcggccggggctagctacgacgcgtgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgaattcatccctgcagcctgcctctgacaagaacccaatgcccaaccctaggccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggccaagcctatggctccttcgctcgcgttagggcctagcccaggagtcttgccttcgtggaaaacagcacccaagggctcagaacttctagggaccaggggctctgggggacccttccaaggtcgggacctgcgaagtggggcccacacctcttcttccttgaaccccctgccaccatcccagctgcaggtgaggcccggggcccagaatggggtaagcagggtggggtacttgggcctataggtgtcgacctttactgtggcatgtggcgggggggggggggggggctggggcacaggaagtggtttatgggtcccaggcaagtctgacttatgcagatattgcagggccaagaaaatccccactctccaggcttcagagattcaaggctttccccacccctcccaatcctcatcccgataggagaccttatgattccatggacatagccatgtatcctcatcccactgtgacgagatggctggggcccaagaaggtaacagtgttggggccagctctaccccttgaaactgttggaccttgatacattcactctccacgagcctcagattccactgatgtgaactggatagttccattgttgctaccgtgtgagactttagtaaagagctaatgaatgagacacaggctagctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 163 - DNA, 189 bp, Artificial Sequence
  DNA sequence of murine FOXP3 1st coding exon included in AAV 1331, 3209 and 3213;  modified to be non-cleavable for TALEN, mT20, mT22 and mT23
gccaagcctatggctccttcgctcgcgttagggcctagcccaggagtcttgccttcgtggaaaacagcacccaagggctcagaacttctagggaccaggggctctgggggacccttccaaggtcgggacctgcgaagtggggcccacacctcttcttccttgaaccccctgccaccatcccagctgcag